SEQUENCE LISTING (258 sequences)

SEQ ID NO: 1 - RNA, 1065 nt, synthetic construct
  source 1..1065: mol_type = other RNA
atgctgtttaatctgaggatcctgttaaacaatgcagcttttagaaatggtcacaacttcatggttcgaaattttcggtgtggacaaccactacaaaataaagtgcagctgaagggccgtgaccttctcactctaaaaaactttaccggagaagaaattaaatatatgctatggctatcagcagatctgaaatttaggataaaacagaaaggagagtatttgcctttattgcaagggaagtccttaggcatgatttttgagaaaagaagtactcgaacaagattgtctacagaaacaggctttgcacttctgggaggacatccttgttttcttaccacacaagatattcatttgggtgtgaatgaaagtctcacggacacggcccgtgtattgtctagcatggcagatgcagtattggctcgagtgtataaacaatcagatttggacaccctggctaaagaagcatccatcccaattatcaatgggctgtcagatttgtaccatcctatccagatcctggctgattacctcacgctccaggaacactatagctctctgaaaggtcttaccctcagctggatcggggatgggaacaatatcctgcactccatcatgatgagcgcagcgaaattcggaatgcaccttcaggcagctactccaaagggttatgagccggatgctagtgtaaccaagttggcagagcagtatgccaaagagaatggtaccaagctgttgctgacaaatgatccattggaagcagcgcatggaggcaatgtattaattacagacacttggataagcatgggacaagaagaggagaagaaaaagcggctccaggctttccaaggttaccaggttacaatgaagactgctaaagttgctgcctctgactggacatttttacactgcttgcccagaaagccagaagaagtggatgatgaagtcttttattctcctcgatcactagtgttcccagaggcagaaaacagaaagtggacaatcatggctgtcatggtgtccctgctgacagattactcacctcagctccagaagcctaaattttga

SEQ ID NO: 2 - DNA, 1065 bp, synthetic construct
  source 1..1065: mol_type = other DNA
atgctgtttaatctgaggatcctgttaaacaatgcagcttttagaaatggtcacaacttcatggttcgaaattttcggtgtggacaaccactacaaaataaagtgcagctgaagggccgtgaccttctcactctaaaaaactttaccggagaagaaattaaatatatgctatggctatcagcagatctgaaatttaggataaaacagaaaggagagtatttgcctttattgcaagggaagtccttaggcatgatttttgagaaaagaagtactcgaacaagattgtctacagaaacaggctttgcacttctgggaggacatccttgttttcttaccacacaagatattcatttgggtgtgaatgaaagtctcacggacacggcccgtgtattgtctagcatggcagatgcagtattggctcgagtgtataaacaatcagatttggacaccctggctaaagaagcatccatcccaattatcaatgggctgtcagatttgtaccatcctatccagatcctggctgattacctcacgctccaggaacactatagctctctgaaaggtcttaccctcagctggatcggggatgggaacaatatcctgcactccatcatgatgagcgcagcgaaattcggaatgcaccttcaggcagctactccaaagggttatgagccggatgctagtgtaaccaagttggcagagcagtatgccaaagagaatggtaccaagctgttgctgacaaatgatccattggaagcagcgcatggaggcaatgtattaattacagacacttggataagcatgggacaagaagaggagaagaaaaagcggctccaggctttccaaggttaccaggttacaatgaagactgctaaagttgctgcctctgactggacatttttacactgcttgcccagaaagccagaagaagtggatgatgaagtcttttattctcctcgatcactagtgttcccagaggcagaaaacagaaagtggacaatcatggctgtcatggtgtccctgctgacagattactcacctcagctccagaagcctaaattttga

SEQ ID NO: 3 - protein, 354 aa, synthetic construct
MLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 4 - protein, 355 aa, synthetic construct
MLVFNLRILLNNAAFRNGHNFMVRNFRCGQPLQNRVQLKGRDLLTLKNFTGEEIRYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 5 - DNA, 129 bp, synthetic construct
  source 1..129: mol_type = other DNA
tcaacacaacatatacaaaacaaacgaatctcaagcaatcaagcattctacttctattgcagcaatttaaatcatttcttttaaagcaaaagcaattttctgaaaattttcaccatttacgaacgatag

SEQ ID NO: 6 - DNA, 28 bp, synthetic construct
  source 1..28: mol_type = other DNA
attattacatcaaaacaaaaagccgcca

SEQ ID NO: 7 - DNA, 245 bp, synthetic construct
  source 1..245: mol_type = other DNA
cttaagggggcgctgcctacggaggtggcagccatctccttctcggcatcaagcttaccatggtgccccaggccctgctcttggtcccgctgctggtgttccccctctgcttcggcaagttccccatctacaccatccccgacaagctggggccgtggagccccatcgacatccaccacctgtcctgccccaacaacctcgtggtcgaggacgagggctgcaccaacctgagcgggttctcctac

SEQ ID NO: 8 - DNA, 177 bp, synthetic construct
  source 1..177: mol_type = other DNA
tgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagtgttgggttgcg

SEQ ID NO: 9 - DNA, 89 bp, synthetic construct
  source 1..89: mol_type = other DNA
aattattggttaaagaagtatattagtgctaatttccctccgtttgtcctagcttttctcttctgtcaaccccacacgcctttggcaca

SEQ ID NO: 10 - DNA, 569 bp, synthetic construct
  source 1..569: mol_type = other DNA
ctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataat

SEQ ID NO: 11 - DNA, 44 bp, synthetic construct
  source 1..44: mol_type = other DNA
gtcagctttcaaactctttgtttcttgtttgttgattgagaata

SEQ ID NO: 12 - DNA, 54 bp, synthetic construct
  source 1..54: mol_type = other DNA
ctctcgcctgagaaaaaaaatccacgaaccaatttctcagcaaccagcagcacg

SEQ ID NO: 13 - DNA, 52 bp, synthetic construct
  source 1..52: mol_type = other DNA
acctgtgagggttcgaaggaagtagcagtgttttttgttcctagaggaagag

SEQ ID NO: 14 - DNA, 71 bp, synthetic construct
  source 1..71: mol_type = other DNA
acacagaaacattcgcaaaaacaaaatcccagtatcaaaattcttctctttttttcatatttcgcaaagac

SEQ ID NO: 15 - DNA, 52 bp, synthetic construct
  source 1..52: mol_type = other DNA
cagaaaaatttgctacattgtttcacaaacttcaaatattattcatttattt

SEQ ID NO: 16 - DNA, 158 bp, synthetic construct
  source 1..158: mol_type = other DNA
ctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacat

SEQ ID NO: 17 - DNA, 166 bp, synthetic construct
  source 1..166: mol_type = other DNA
tgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccat

SEQ ID NO: 18 - DNA, 143 bp, synthetic construct
  source 1..143: mol_type = other DNA
acgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 19 - DNA, 220 bp, synthetic construct
  source 1..220: mol_type = other DNA
tagagcggcaaaccctagctacactccatagctagtttcttttttttttgtttttttttttttttttttttttttttttttttttttttcctttcttttccttctttttttcctcttttcttggtggctccatcttagccctagtcacggctagctgtgaaaggtccgtgagccgcatgactgcagagagtgccgtaactggtctctctgcagatcatgt

SEQ ID NO: 20 - DNA, 170 bp, synthetic construct
  source 1..170: mol_type = other DNA
acacatcacaaccacaaccttctcaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctac

SEQ ID NO: 21 - DNA, 110 bp, synthetic construct
  source 1..110: mol_type = other DNA
gctggagcctcggtagccgttcctcctgcccgctgggcctcccaacgggccctcctcccctccttgcaccggcccttcctggtctttgaataaagtctgagtgggcagca

SEQ ID NO: 22 - DNA, 123 bp, synthetic construct
  source 1..123: mol_type = other DNA
tagtgcagtcactggcacaacgcgttgcccggtaagccaatcgggtatacacggtcgtcatactgcagacagggttcttctactttgcaagatagtctagagtagtaaaataaatagtataag

SEQ ID NO: 23-25 - skipped (3 entries)

SEQ ID NO: 26 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgtttaatctgaggatcctgttaaacaatgcagcttttagaaatggtcacaacttcatggttcgaaattttcggtgtggacaaccactacaaaataaagtgcagctgaagggccgtgaccttctcactctaaaaaactttaccggagaagaaattaaatatatgctatggctatcagcagatctgaaatttaggataaaacagaaaggagagtatttgcctttattgcaagggaagtccttaggcatgatttttgagaaaagaagtactcgaacaagattgtctacagaaacaggctttgcacttctgggaggacatccttgttttcttaccacacaagatattcatttgggtgtgaatgaaagtctcacggacacggcccgtgtattgtctagcatggcagatgcagtattggctcgagtgtataaacaatcagatttggacaccctggctaaagaagcatccatcccaattatcaatgggctgtcagatttgtaccatcctatccagatcctggctgattacctcacgctccaggaacactatagctctctgaaaggtcttaccctcagctggatcggggatgggaacaatatcctgcactccatcatgatgagcgcagcgaaattcggaatgcaccttcaggcagctactccaaagggttatgagccggatgctagtgtaaccaagttggcagagcagtatgccaaagagaatggtaccaagctgttgctgacaaatgatccattggaagcagcgcatggaggcaatgtattaattacagacacttggataagcatgggacaagaagaggagaagaaaaagcggctccaggctttccaaggttaccaggttacaatgaagactgctaaagttgctgcctctgactggacatttttacactgcttgcccagaaagccagaagaagtggatgatgaagtcttttattctcctcgatcactagtgttcccagaggcagaaaacagaaagtggacaatcatggctgtcatggtgtccctgctgacagattactcacctcagctccagaagcctaaattttgactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 27 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctctttaatctgcgcatcttactgaacaacgccgcattccggaacggtcacaacttcatggtccgcaatttccgctgtggccagccgcttcaaaacaaggtccagctgaagggacgggatctgctgacactgaagaacttcaccggagaagagatcaagtacatgctgtggctcagcgcagacttgaagttccggatcaagcagaagggagaatacttgcccctgctgcaaggaaagtcgctgggaatgatttttgagaagcggtcaactcgcaccagactctccaccgaaactggtttcgcactgcttggcgggcacccttgcttcctgacgactcaggacatccacctcggcgtgaacgaatcgctaaccgataccgccagagtgctttcttccatggccgacgcggtgctggccagggtgtacaagcagtccgacctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcgtccgtgaccaagttggcggaacagtacgcgaaggagaacggaaccaagcttctgctgactaacgaccccctcgaggctgcgcatgggggcaacgtgctgattaccgacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 28 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgtttaacctacgtattttgctcaacaatgcagcctttagaaacggacataactttatggttcgaaactttcgctgcgggcagccactgcagaacaaggtccagctgaaagggagagatttgctcacgctgaagaactttactggcgaagaaatcaagtatatgctgtggttgtccgcggacctcaagtttcggattaagcagaaaggggagtatctgccactgctgcaaggaaagagcctcggcatgatcttcgagaagcggagcactcggaccaggctgagtaccgaaactggcttcgcattgttgggtggacatccatgttttctgacaacgcaggacattcatctgggcgtgaacgagagtctgacggacacagctcgcgttctgtcctctatggctgatgcggtgttggcccgggtctataagcagtccgatttggacaccttggctaaggaagctagcataccgattatcaatgggctgtccgacctgtatcaccctattcaaatcctggccgactacctcacactgcaagaacactatagctcattgaagggactgaccctgagctggataggggacggaaacaacatcctacatagcattatgatgtccgctgccaagtttggcatgcatcttcaagccgccacgccaaagggttatgagcccgacgcgtcagtgacaaagctggccgagcagtacgctaaggagaatggtaccaaattactgctgactaatgatccactggaggctgcacatggcggcaatgtactgatcaccgacacatggatctcgatgggccaggaggaagaaaagaagaagaggcttcaggccttccaaggctaccaggtcaccatgaaaacagctaaggttgcagcatctgattggacctttctgcactgtctgccaaggaagcccgaagaggtggacgatgaagtattctatagcccacggagtttggtgttccctgaggctgaaaataggaagtggacaattatggccgtaatggtgtccctgttaaccgactactctccgcaactgcagaaacctaagttttagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 29 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgtttaacttaaggatcctgctgaacaacgccgcttttcgtaacggtcataactttatggtccggaactttagatgtggccagccgctgcagaacaaggttcagctgaaggggagggatctgctgaccttgaagaactttaccggcgaagagatcaagtacatgttgtggctgagcgccgatctgaagtttaggattaagcagaagggggagtatttgccactgctgcaaggaaaatccttggggatgatcttcgagaagcgctccactagaacccggctaagcacagaaaccggcttcgcacttctgggtggacatccctgttttctgacgacgcaggatatacacctgggcgtgaatgagagtctgacggacacagctagggtgttgagcagcatggccgatgcagtactggcccgcgtttataagcagagcgacttggacacactggccaaggaagcgtcaattccgattatcaatgggctgtcagacctgtatcatcccattcaaatcttggctgactatctgaccctgcaagaacattacagctccctgaagggcctcacgttgtcctggattggcgacggaaacaacattctgcattcgatcatgatgagcgctgctaagtttggcatgcacctccaagccgctacacctaagggatatgagcctgatgccagcgtaaccaagctggccgaacagtacgcgaaggagaatggcacgaaactgctgttgacaaatgacccactggaggcagctcacggtggcaacgtgctgatcaccgacacgtggatatctatgggacaggaagaagagaagaagaagcggctgcaggcattccaagggtatcaggtcaccatgaaaacggccaaggttgctgcatccgactggacatttctgcattgcttgccccgcaaaccagaagaagtagacgacgaagtcttttattccccacggtcgctggtgttccccgaggcggagaatcgaaagtggacgattatggccgtgatggtgtccctgctgactgattactctccccaactgcaaaagcctaagttttagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 30 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaacctgaggatcctcctgaacaacgccgcctttcgcaatggtcacaactttatggtccggaacttcagatgcggccagccgctgcagaacaaggtccagctgaagggacgggatctgctgactctgaagaacttcaccggagaagagatcaagtacatgctgtggctgtcggccgacctgaagttcaggatcaagcagaagggagaatacctcccgctgctgcaaggaaagtccctgggcatgattttcgagaagcgctcgaccagaactcggttgtccaccgaaaccgggtttgcgctgctgggcggacatccttgcttcctgacgactcaggatattcacctgggagtgaacgagtcgctgaccgacaccgccagagtgctgagctcgatggccgacgccgtgttggcacgcgtgtacaagcagtccgatctggataccctggccaaagaagcttccatcccgatcattaacgggctgagcgacctctaccaccccattcaaatcctggccgactacctgactctgcaagaacactacagctcgctgaaggggttgactctgtcctggatcggcgacggaaacaacatcctgcactccatcatgatgtcggccgcaaagttcggcatgcatttgcaagccgccaccccaaagggctacgaaccagacgcgagcgtcaccaagctggccgaacagtacgcgaaggaaaatggtactaagctgctgctgaccaacgacccattggaagctgcccatggtggaaacgtgctgatcaccgacacctggatctcgatgggccaggaagaggagaagaagaagcggctgcaggcgttccaggggtatcaggtcaccatgaaaacagccaaagtggcagcgtcagactggaccttcctccactgtctgcctcgcaagccagaggaggtggacgacgaggtgttctactcccctcggtccctcgtgttccctgaggctgagaaccggaagtggaccattatggccgtgatggtgtcactcctgactgattactccccgcaactgcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 31 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgtttaacctgaggatcctattgaacaatgctgcttttcgtaatggccataactttatggttcggaactttagatgcgggcagccactgcagaacaaggtccagttgaaaggccgcgatctgttgacattgaagaactttaccggcgaagagattaagtatatgctgtggctgtctgctgacctcaagtttcgaatcaagcagaagggcgaatatctccccctgctgcaaggaaagtctctcggcatgatctttgagaagcggagtacccgaacacggctgagcaccgaaacgggcttcgcactgctggggggccatccctgttttctgacaacgcaggacatccacttgggggttaacgaatcattgactgataccgcccgcgtactgtcatccatggccgacgctgtgctggctagggtgtacaagcagtcagatctggatacactggccaaggaagctagcataccaatcatcaatggactgagtgacctttatcacccgattcaaatactagccgattatctgaccctgcaagagcattactcctcgctgaaaggcctcacgctgtcctggatcggcgacggcaacaacattctgcatagtattatgatgtctgctgccaaattcggcatgcatctgcaagctgctacgccgaagggttatgaacccgacgcgtcagttacgaagctcgctgagcagtatgcaaaggagaatggcacaaagctgttgcttaccaacgatcccctggaagctgctcatggcggcaatgtgctgattactgacacctggatttcaatgggccaggaggaggagaagaagaagaggttacaggcttttcaaggttaccaagtcacgatgaaaaccgctaaggtcgcagccagcgactggacattcctgcactgtctgccaagaaagccggaagaagtggacgacgaggtgttctattccccgcggtctttggtgtttccggaggccgaaaacaggaaatggaccattatggccgtgatggtatcgttgctgacggactacagccctcagttgcaaaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 32 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctctttaacctccgcatcctcctcaacaacgccgccttccggaatgggcataacttcatggtccggaacttcagatgcggccagcccctgcaaaacaaggtccagttgaagggacgggacctccttacgctgaagaactttaccggagaagagattaagtacatgctgtggttgtccgctgacctcaagttccgcattaagcagaagggagaatatctgccgctgctgcaaggaaagagcctgggcatgatcttcgaaaagcgctccactagaacccggctgtcgactgagactggattcgccttgctcggtggacacccgtgcttcctgacgacccaggacatccacctgggagtgaacgagtcacttacggataccgcgagggtgctgtcctcaatggccgacgcagtgctcgcgcgcgtgtacaagcagtcagatctggataccctggccaaggaagccagcattcccatcatcaacggactgagcgacctttaccacccaatccagatcctcgccgactacttaaccctgcaagagcactacagctccctgaagggactgactctgtcctggatcggggatggaaacaacatcctgcactccatcatgatgtctgccgctaagtttgggatgcatctgcaagccgcaacccctaagggatacgagcccgacgcctcggtgaccaaacttgcggaacagtacgccaaggaaaacggtaccaagctgctgctgaccaacgaccctctggaagcggcccacggaggaaatgtgctgattaccgacacctggatttcgatgggccaggaggaggagaagaagaagagactgcaggcgttccagggatatcaggtcaccatgaaaaccgccaaggtcgccgccagcgactggaccttcctgcactgtctccctcggaaaccggaagaagtggatgacgaggtgttctactccccgcgctcgctggtgttcccggaggctgaaaacaggaagtggacaatcatggccgtgatggtgtccctgttgaccgactactccccacaactgcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 33 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatctgcgcatcctcctgaacaacgccgccttccgcaatggacacaactttatggtccgcaacttccgctgtgggcagccgctgcagaacaaggtccagctcaaggggagagatctcctgaccctgaagaacttcactggagaggagatcaagtacatgctgtggctgtccgccgacctgaaatttcggattaagcagaagggcgaatacctcccactgctgcaaggaaagtctttgggcatgatcttcgaaaagagaagcacccggacccggttgagcaccgaaactgggttcgcgctcctcggtggacacccgtgcttcctgaccacccaagatattcatctgggtgtcaacgaaagcctgaccgacaccgccagggtgctgtcatccatggctgacgcagtgctcgcccgggtgtacaagcagtcagacctggacaccctcgccaaggaagcttcgatccctatcatcaacggactttccgacctgtaccaccccatccaaattctggccgactacctgactctgcaagaacactatagctcgctgaaaggacttactctgtcctggatcggggacggcaacaacattctccattccatcatgatgtccgctgccaagttcggaatgcaccttcaagcagcgactcccaagggatacgaacctgatgcctccgtgactaagctggcagagcagtacgccaaggagaacggtacaaagctgctgctcacgaacgaccccctggaggcggcccacggcggaaacgtgctgattaccgatacctggatctcaatgggccaggaagaggagaagaagaagcggctccaggcgtttcaaggctaccaggtcaccatgaaaaccgcgaaggtcgccgcctccgactggactttcttgcactgcctgccgcggaagcccgaggaagtggatgacgaagtgttctactcgccgagatcgttggtgttccctgaggccgaaaacaggaagtggaccatcatggccgtgatggtgtccctgctgactgattacagcccacagctgcagaagcctaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 34 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 35 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaacctgagaatcctcctgaacaacgccgccttccgcaatggtcataacttcatggtccgcaactttcgctgcggacagcctctccaaaacaaggtccagctcaaggggcgcgacctcctcacactgaagaacttcactggagaagaaatcaagtacatgctgtggctgagcgccgatctgaagttccggatcaagcagaagggagagtaccttcctctgctgcaagggaagtccttgggaatgattttcgagaagcggtccacccggaccaggctgagcactgaaactggcttcgccctgctgggaggccacccttgtttcctgaccactcaggacatccacctgggcgtgaacgagtccctgaccgatactgccagagtgctgtcctccatggccgacgccgtgctcgcccgggtgtacaagcagtcagacctcgatacgctggccaaggaagcctccattcccattatcaatggtctgtcggacctctaccatccaatccaaatcctcgccgactacctgactctgcaagaacactacagctcactcaagggcctcaccctctcctggatcggcgacggaaacaacatccttcactcgattatgatgtcggccgcgaagttcgggatgcacctccaagctgccactccaaaaggctacgagccggatgcctcagtgactaagttggcggaacagtatgcgaaggagaacggtaccaagctcctgctgactaacgacccgctggaggccgcccacgggggaaacgtgctcatcaccgatacttggatttccatgggacaggaggaagagaagaagaagcggttgcaggcatttcagggctaccaggtcaccatgaaaactgccaaagtcgccgccagcgactggaccttcctgcactgcctgccccgcaagcctgaagaagtggacgacgaggtgttctactctccccggtccctcgtgttccctgaggccgaaaacaggaagtggaccatcatggctgtgatggtgtccctcctgaccgactacagccctcagctccaaaaacccaagttttagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 36 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaacctgagaatcctcttgaacaatgctgcttttcggaatggccacaactttatggttcggaacttccgttgcggccagcctttacaaaacaaggtccagctgaagggccgggatttgctcacactgaagaactttactggggaggagattaagtatatgctgtggctgtccgctgacctgaagtttaggatcaagcagaagggcgaatatctgccgctgctgcaagggaaaagtctgggcatgatttttgaaaagcgctctacccggaccagactgtctacggaaacaggctttgccctgctgggcggccacccctgttttctgacaacgcaggacatccatctgggcgtgaacgaatcactgaccgatactgctcgggtactcagttctatggctgacgcagtgctggctagggtgtacaagcagagcgacttggacacactggctaaggaggccagcatccccattatcaatggcctgtctgatttgtaccatcccattcaaatcctggctgattatctgacactacaagagcattactcaagtctgaagggtttgactctctcctggatcggcgacggcaacaacattttacattccattatgatgagtgctgctaagtttggcatgcatttgcaagctgctaccccaaagggctatgaacctgacgctagcgtaaccaagttggccgaacagtatgctaaagagaatggcaccaagctgctcctgacgaatgaccccctggaagctgctcatggcggaaacgtacttataactgatacatggattagcatgggccaggaagaggagaagaagaagagactgcaggccttccaaggctatcaggtcaccatgaaaactgccaaggttgcagctagcgactggaccttcctgcactgtttgccgaggaaacccgaggaggtggacgatgaagtcttttattctccccgctccttggtgtttcccgaggctgaaaatcgaaagtggacgataatggcagtgatggtgtccctactgaccgactattctccacaactgcagaagcctaaattctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 37 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatctgaggatcctgctgaacaacgctgcttttcgcaacggtcataactttatggttcgcaattttcgttgtggccagccgctgcagaacaaggttcagctgaagggcagagatctgctgactctgaagaacttcactggggaagaaatcaagtatatgttatggctgtccgcggatctgaaatttcgaatcaagcagaagggcgaatatcttcccctgctgcaagggaaatccttgggcatgatttttgagaagaggagcactaggactagattgtcaacagaaacaggctttgctttgttgggcggacatccctgctttctgacgacacaggatatccacctcggcgtaaacgagtccctcaccgacactgctagggtactgagcagcatggccgacgctgtgctagcccgggtttacaagcagtcagacctggacacccttgccaaggaagcttctattccaattatcaacggcctgagtgacctgtatcaccctattcaaatactcgccgactatttgacgcttcaagaacattacagcagcctcaagggcttaaccttgagttggataggcgacggcaacaatatcctgcattccattatgatgtctgccgctaagtttggcatgcatctacaagccgcaacacccaagggctatgaacccgacgctagcgtgaccaagctggccgagcagtatgctaaggaaaatggcacaaagctccttcttaccaacgatcccctggaggctgctcacggcggcaacgtgctgattaccgatacatggattagcatgggccaggaggaggagaaaaagaagcggctccaggcttttcaaggctatcaggtcaccatgaaaactgcaaaggtcgctgcctccgactggactttcctgcattgtctaccccgcaagcctgaggaagtggacgatgaggtgttctactccccacggagtctggtgttcccggaagcagagaatcggaagtggaccatcatggctgtcatggtgtcgctcttgactgactattctccccaactgcaaaaacccaagttttagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 38 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgttattcaaccttcgtatcctgctaaacaatgctgcttttcgcaatggccataactttatggttcgcaactttagatgcggccagccgctgcagaacaaggttcagctgaagggccgggacttgctgacgctgaaaaactttaccggggaagagattaagtatatgctgtggctaagcgctgatctgaagtttaggatcaagcagaagggcgaatatctgccactgctgcaagggaagagtcttggcatgatttttgaaaagcggtctaccagaacccggctgtcgaccgagacaggttttgctctgctggggggccatccctgttttctgacaactcaggacattcacctgggcgtgaatgagtccctgaccgatactgctagggtgttgagtagcatggccgacgctgtactcgctcgagtgtataagcagtctgatctggacactctggctaaggaagcttccattcctattatcaacggcttgagcgacctgtaccaccccattcaaatcctcgctgattacttgactttgcaagaacattacagcagcttgaagggcttaacactgagctggataggcgacggaaacaacatcttgcattccataatgatgtccgccgctaagttcgggatgcacctccaagcagccacacccaagggctatgaaccggatgcttccgtgacaaaactggctgagcagtatgctaaggagaatggcacgaaactgctgctcaccaacgacccattggaagctgcacatggtggcaacgtactgatcactgacacttggatctcaatgggccaggaggaagagaagaagaaaaggctgcaggcatttcagggataccaagtcactatgaaaactgccaaggtcgctgcctccgactggacattcctgcattgtctgccacggaagcctgaggaagtcgatgacgaagtgttctatagcccacgaagcttggtgtttcccgaggctgagaataggaagtggaccattatggctgttatggtgtccctgctcaccgactattcccctcaactgcaaaaacccaagttttagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 39 - RNA, 1365 nt, synthetic construct
  source 1..1365: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatgctttttaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccacgccaaaaggatacgaaccggatgcgtccgtgacgaagttggcggaacagtacgcgaaggagaacggaaccaagcttctgctgactaacgaccccctcgaggctgcgcatgggggcaacgtgctgattaccgacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 40 - RNA, 1365 nt, synthetic construct
  source 1..1365: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatgcttttcaacctgagaatcctcttgaacaatgctgcttttcggaatggccacaactttatggttcggaacttccgttgcggccagcctttacaaaacaaggtccagctgaagggccgggatttgctcacactgaagaactttactggagaagagatcaagtacatgctgtggctgtcggccgacctgaagttcaggatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcgtccgtgaccaagttggcggaacagtacgcgaaggagaacggaaccaagcttctgctgactaacgaccccctcgaggctgcgcatgggggcaacgtgctgattaccgacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 41 - RNA, 1365 nt, synthetic construct
  source 1..1365: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcatccgtgaccaagttggcggaacagtacgcgaaggagaacggaaccaagctcctgctgactaacgacccgctcgaggctgcgcatgggggtaacgtgctgattacggacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 42 - RNA, 1365 nt, synthetic construct
  source 1..1365: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcgtccgtgaccaagttggcggaacagtacgcgaaggagaacggaaccaagcttctgctgactaacgaccccctcgaggctgcgcatgggggcaacgtgctgattaccgacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 43 - RNA, 1371 nt, synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgttcaacctgcgaatcctgctgaacaacgccgcttttcggaacgggcacaactttatggtgaggaactttcgctgcggacagcccctccagaataaggtccagctgaagggcagggacctgctgaccctgaaaaatttcacaggggaggaaatcaagtatatgctgtggctgtcagctgatctgaagttccggatcaagcagaagggcgaatatctgcctctgctccagggcaaaagcctggggatgatcttcgaaaagcgcagtactcggaccagactgtcaaccgagactggattcgctctgctgggaggacacccttgttttctgaccactcaggacattcacctgggagtgaacgagtccctgaccgacactgctcgcgtcctgagctctatggccgacgctgtgctggctcgagtctacaaacagtccgacctggataccctggccaaggaagcttctatcccaattattaacggcctgtcagacctgtatcaccccatccagattctggccgattacctgaccctccaggagcactattctagtctgaaagggctgacactgagttggattggggacggaaacaatatcctgcactctattatgatgtcagccgccaagtttggaatgcacctccaggctgcaaccccaaaaggctacgaacccgatgcctcagtgacaaagctggctgaacagtacgccaaagagaacggcactaagctgctgctgaccaacgaccctctggaggccgctcacggaggcaacgtgctgatcaccgatacctggattagtatgggacaggaggaagagaagaagaagcggctccaggccttccagggctaccaggtgacaatgaaaaccgctaaggtcgcagccagcgattggacctttctgcactgcctgcccagaaagcccgaagaggtggacgacgaggtcttctactctcccagaagcctggtgtttcccgaagctgagaataggaagtggacaattatggcagtgatggtcagcctgctgactgattattcacctcagctccagaaaccaaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 44 - RNA, 1371 nt, synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 45 - RNA, 1371 nt, synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgttcaacctgcgaatcctgctgaacaacgccgcttttcggaacgggcacaactttatggtgaggaactttcgctgcggacagcccctccagaataaggtccagctgaagggcagggacctgctgaccctgaaaaatttcacaggggaggaaatcaagtatatgctgtggctgtcagctgatctgaagttccggatcaagcagaagggcgaatatctgcctctgctccagggcaaaagcctggggatgatcttcgaaaagcgcagtactcggaccagactgtcaaccgagactggattcgctctgctgggaggacacccttgttttctgaccactcaggacattcacctgggagtgaacgagtccctgaccgacactgctcgcgtcctgagctctatggccgacgctgtgctagctcgagtctacaaacagtccgacctggataccctggccaaggaagcttctatcccaattattaacggcctgtcagacctgtatcaccccatccagattctggccgattacctgaccctccaggagcactattctagtctgaaagggctgacactgagttggattggggacggaaacaatatcctgcactctattatgatgtcagccgccaagtttggaatgcacctccaggctgcaaccccaaaaggctacgaacccgatgcctcagtgacaaagctggctgaacagtacgccaaagagaacggcactaagctgctgctgaccaacgaccctctggaggccgctcacggaggcaacgtgctgatcaccgatacctggattagtatgggacaggaggaagagaagaagaagcggctccaggccttccagggctaccaggtgacaatgaaaaccgctaaggtcgcagccagcgattggacctttctgcactgcctgcccagaaagcccgaagaggtggacgacgaggtcttctactctcccagaagcctggtgtttcccgaagctgagaataggaagtggacaattatggcagtgatggtcagcctgctgactgattattcacctcagctccagaaaccaaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 46 - RNA, 1371 nt, synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgttcaacctgcgaatcctgctgaacaatgccgcttttcggaacgggcacaatttcatggtgaggaactttcgctgcggacagcccctccagaacaaggtccagctgaagggcagggacctgctgaccctgaaaaatttcacaggggaggaaatcaagtacatgctgtggctgtcagccgatctgaagttccggatcaagcagaagggcgaatatctgcctctgctccagggcaaaagcctggggatgatcttcgaaaagcgcagtactcggaccagactgtcaacagagactggattcgcactgctgggaggacacccatgttttctgaccacacaggacattcatctgggagtgaacgagtccctgaccgacacagcacgcgtcctgagctccatggctgatgcagtgctggctcgagtctacaaacagtctgacctggataccctggccaaggaagcttctatcccaatcattaatggcctgagtgacctgtatcaccccatccagattctggccgattacctgaccctccaggagcattattctagtctgaaagggctgacactgagctggattggggacggaaacaatatcctgcactccattatgatgagcgccgccaagtttggaatgcacctccaggctgcaaccccaaaaggctacgaacccgatgcctccgtgacaaagctggcagaacagtatgccaaagagaacggcactaagctgctgctgaccaatgaccctctggaggccgctcacggaggcaacgtgctgatcactgatacctggattagtatgggacaggaggaagagaagaagaagcggctccaggccttccagggctaccaggtgacaatgaaaactgctaaggtcgcagccagcgactggacctttctgcattgcctgcccagaaagcctgaagaggtggacgatgaggtcttctactcacccagaagcctggtgtttcctgaagctgagaataggaagtggacaatcatggcagtgatggtcagcctgctgactgattattcccctcagctccagaaaccaaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 47 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaaccttcgcattctcctcaacaacgccgcgtttagaaacggacacaacttcatggtccgcaacttccgctgcggacagccgctgcagaacaaggtccagctcaagggtcgggatctcctgacgctgaagaactttaccggcgaagagattaagtacatgctgtggctgtccgccgaccttaagttccggatcaagcagaagggcgaataccttcccctgctgcaaggaaagtccctgggcatgatcttcgagaagcgcagtaccagaaccagactctccactgaaaccgggttcgcgctgcttggcggccacccgtgtttcctcactacgcaagacatccatcttggcgtgaacgagtcccttaccgacaccgccagggtgctgtcaagcatggccgacgccgtccttgcgcgcgtgtacaagcagtcagaccttgatactctggccaaggaagcctccatccctattatcaacggcctatccgacctttaccacccgatccagatcctcgctgactacctgaccctgcaagaacactacagcagcctcaagggactgactctgtcctggatcggcgacgggaacaacatcctgcactcaatcatgatgagcgcagccaagttcggcatgcatctccaagccgctacacccaagggttatgaaccggacgcctctgtgaccaagttggcagaacagtacgccaaggagaacggtactaagctccttttaaccaacgaccccctcgaagcagcccatggcgggaatgtgctcattaccgatacctggatttcgatgggccaggaggaggagaagaagaagcggctgcaggcgttccagggctaccaggtcaccatgaaaactgccaaagtggccgcctcggattggacctttctccactgcctgcctcggaagcctgaggaggtggacgacgaagtgttctactccccacggtccctcgtgttccccgaggccgaaaataggaagtggaccatcatggccgtgatggtgtccctcttgaccgattacagcccgcagcttcagaagcctaaattctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 48 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatcttcgcatcctgttgaacaacgccgccttccgcaatggtcacaacttcatggtccggaacttcagatgtggacagcctctccaaaacaaggtccagctgaagggaagggacctcttaaccctcaaaaactttactggagaggagatcaagtacatgctgtggcttagcgccgaccttaagttccggatcaagcagaagggagagtacctcccgctgctgcaaggaaagagtcttggaatgatcttcgagaagcggtccaccagaactcgcctctccactgaaaccggattcgcactcctgggtggacacccgtgctttctgaccacccaagacatccacctcggagtgaacgagagcctcacggacaccgcgagagtgctgtcatccatggccgacgccgtgcttgcacgggtctacaagcagtccgatctggacactcttgccaaggaagcctccattcctatcattaacggtctgtcggatctgtaccacccgattcagatccttgcggactacctcacacttcaagaacactattcaagcctaaagggtctgaccctgtcctggatcggagatggaaacaacattctccattccatcatgatgagcgctgccaagttcggaatgcatctccaagcagcgactcctaagggttacgagccggacgcctcagtgactaagctggccgagcagtacgccaaggagaacggtaccaaactgttgcttactaacgacccgcttgaagcggcccatggaggaaacgtgctgattaccgacacctggatttcgatgggacaggaagaggagaagaagaagcggctccaggcgttccagggataccaggtcaccatgaaaacggccaaagtggccgctagcgattggacctttctgcactgcctcccgcgcaagcctgaagaagtggacgacgaagtgttctactcccctcgctctcttgtgttcccggaagccgaaaacaggaagtggaccatcatggccgtgatggtgtccctcctgaccgattacagcccgcagctgcagaagcctaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 49 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatctccgcatcctcctcaacaacgccgcgtttagaaacggccacaacttcatggtccggaacttcagatgtggccagccgcttcagaacaaggtccagctcaagggccgggatcttctgaccctgaagaactttactggcgaagaaatcaagtacatgctctggctctccgccgacttgaagttccgcattaagcagaagggggaataccttccgctgctgcaaggaaagtcgctcggcatgatctttgagaagcgctcaacccgcaccaggctgtccactgaaaccgggttcgcgctgcttggtggccacccctgcttcctgaccacccaagacattcacctcggagtgaacgaatcgctcactgatactgcccgggtgctgtcgtcgatggccgatgcagtgctggccagggtgtacaaacagtccgatctggacactctggccaaggaggcgtccatccctattatcaacggcctttccgacctctaccacccgattcagatccttgccgattacctcaccctgcaagaacactactcgtcactgaagggtctgaccttgtcctggatcggcgacggcaacaacatcctccattccattatgatgtccgccgccaaattcggcatgcatcttcaagccgcaacccctaagggttacgagccggacgcttccgtgaccaagctcgccgagcagtacgctaaggagaacggaaccaagcttctgctgactaacgaccccctagaggcagcccacgggggcaacgtgcttattactgacacctggatctccatgggacaggaagaagagaagaagaagcggttacaggcgttccagggctatcaggtcaccatgaaaaccgccaaggtcgctgcctcggactggaccttcctgcattgcctgcctcgcaagcccgaagaagtggacgacgaggtgttctactcgccacggtcccttgtgttccctgaggccgagaatagaaagtggaccattatggccgtgatggtgtcccttctcaccgactactcgccgcaactgcagaaacccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 50 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatcttcgcatcctcctcaacaacgccgccttccggaacggtcacaacttcatggtccggaacttccgctgcggccagccgctccaaaacaaagtgcagcttaagggccgcgatctcctgaccctgaagaacttcaccggagaggaaatcaagtacatgctgtggctctcggcggacctgaagtttaggattaagcagaagggggagtatctgccgctgctccaagggaagtcccttggcatgatcttcgaaaagaggtccacccggactcggctcagcaccgaaacaggttttgcacttctggggggccacccgtgcttcctgacgacccaggacatccatctgggtgtcaacgagagtttgaccgacactgccagagtgctgtcatccatggcggacgcggtgctcgcgagagtgtacaagcagtccgatcttgacaccctggcaaaagaggcttcaatcccgatcattaacggactctcggatctgtaccaccctatccaaatcttggccgactacctgaccctgcaagaacactacagctccctgaagggcctgactctttcctggattggcgatggaaacaacattctccattctattatgatgtccgccgccaagttcggcatgcaccttcaagccgccaccccgaagggctacgaacctgacgcctccgtgactaagctagccgaacagtacgctaaggagaacggcactaagcttctccttaccaacgatccgctggaggcggcccatggcggaaatgtgcttatcaccgacacctggattagcatggggcaggaagaagagaagaagaaacggctccaggcattccagggctaccaggtcaccatgaaaactgccaaggtcgccgctagcgactggaccttcctccactgtctgcctcgcaagcctgaagaagtggacgacgaggtgttctactccccgcgctccctcgtgtttcctgaggccgagaacagaaagtggaccatcatggccgtgatggtgtcattacttacggactacagcccgcagctgcagaagccgaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 51 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctttttaacttgagaatccttctgaacaacgccgctttccgcaacggtcataacttcatggtccggaacttcagatgtggccagcccctccaaaacaaagtgcagctgaagggccgggaccttcttacgctgaagaatttcaccggcgaagaaatcaagtacatgctctggctgtccgccgatcttaagttccgcattaagcagaagggggaatacctcccgctgctgcaagggaagtcgctgggcatgatttttgagaagcggtcaactcgcacccgcctgtccactgaaactggattcgcactgctcggtggccatccctgcttcctgaccacccaagacatccacctcggcgtgaacgagtccctgactgacaccgcccgggtcttatcctcgatggccgatgctgtgcttgcgagggtgtacaagcagtccgacctcgacacactcgcgaaggaggcctccatccccatcatcaacggcctgtccgacctttaccacccaattcagatcctcgccgattacctgaccctgcaagagcactactcgtcgctcaaggggcttaccctctcgtggattggcgacggcaacaacatccttcactccatcatgatgtcggcagcgaagttcggcatgcatctgcaagccgccacgcctaagggttatgaaccggatgcctcagtgaccaagctcgccgaacagtacgcgaaagagaatggaaccaagctacttctgaccaacgaccccctggaggccgctcacggcggcaacgtcctcattaccgatacttggatttcgatgggacaggaagaggaaaagaagaagagactgcaggcgttccagggataccaggtcaccatgaaaactgccaaagtggcagcctccgactggaccttccttcactgcctgccgaggaagcctgaagaggtggacgacgaggtgttctactccccgcgctccttggtgtttcctgaggccgaaaaccggaagtggactatcatggccgtgatggtgtccctcctcaccgactactcgccgcaactgcagaagcctaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 52 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgttattcaaccttagaattctccttaacaacgccgccttccggaatgggcataactttatggtccgcaatttccgctgtggacagcctctgcaaaacaaggtccagctcaagggccgggatctgctgactctcaagaacttcactggggaagaaatcaagtacatgctctggctgagcgccgacctcaagttccgcatcaagcagaagggagagtacctcccgctgctccaagggaagtccctgggcatgatcttcgagaagagatccacccgcaccagactttccactgagactggcttcgccttgctgggaggccacccatgcttcctgacgacccaggacattcaccttggcgtgaacgagtccctgactgacaccgcaagggtgttgtcctcgatggccgacgccgtgcttgcccgggtgtacaagcagagcgatcttgacaccctggctaaggaagcttccattcccatcatcaacggtctgagcgacctgtaccacccgattcagatcctggcggactacctaaccctgcaagagcactatagctccctgaagggcctcacactttcatggatcggcgacggcaacaacatcctgcactctattatgatgagcgctgccaaattcggcatgcacctccaagccgccacgcctaaaggctacgagcccgacgcctcggtgaccaagcttgcggagcagtacgcgaaggaaaacggcaccaagctgcttctcaccaacgatcctctggaagcggcccatggtggcaacgtgctcattaccgacacttggatctccatgggacaggaggaggaaaagaagaagcggctccaggcgtttcagggttaccaggtcaccatgaaaaccgccaaggtcgcagcctccgactggaccttccttcattgccttccgcgcaagcccgaagaagtggacgatgaagtgttttactcacctcggtcactcgtgttcccggaagcagagaacaggaaatggaccattatggccgtgatggtgtccctgctcaccgattacagtccgcaactgcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 53 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcaaatcctggccgattacctcaccctgcaagaacactacagctccctgaagggtctgacattgtcctggatcggcgacggcaacaacattctccattccatcatgatgtccgccgcaaaattcggcatgcatcttcaagccgccacgcctaagggttacgaacccgacgcttccgtgactaagctcgccgagcagtacgctaaggagaacggaaccaagctgctgctgactaacgacccgctagaagcagcccacgggggcaacgtgcttattactgacacctggatctccatgggccaggaggaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcacttctcaccgactacagcccgcagcttcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 54 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatctccgcatcctccttaacaacgccgcgtttagaaacggccacaacttcatggtccggaacttcagatgtggccagccgcttcaaaacaaggtccagcttaagggccgggatctcctcacccttaaaaacttcaccggcgaagagatcaagtacatgctctggctctccgcggaccttaagttccgcattaagcagaagggggaataccttccgctgcttcaaggaaagagcctcggcatgatctttgagaagcgctcaaccaggaccaggctttctactgaaactgggttcgcgcttctcggcggtcatccctgcttcctcacgacccaagacatccacctcggagtgaacgaatccctcacggatactgcccgcgtgctttcgagcatggcagacgccgtgctcgcccgggtgtacaaacagtccgatctcgacactctcgccaaggaggcgtcaattcctattatcaacggtcttagtgacctttaccacccgatccagatcctcgccgattacctcacactccaagaacactacagctcccttaagggtcttaccctctcctggatcggcgacggcaacaacattctccactccatcatgatgtccgccgcaaagttcggcatgcatcttcaagccgccaccccgaagggctacgagcctgatgcttccgtgactaagctcgccgagcagtacgctaaggagaacggaaccaagcttcttctcactaacgacccactcgaagcagcccatgggggcaacgtgcttatcactgacacctggatctccatgggccaggaagaagagaagaagaagcggctccaggcgttccagggatatcaggtcaccatgaaaaccgccaaggtcgctgcctccgactggacctttctccactgcctccctcgcaaacctgaagaagtggacgacgaggtgttctactcgccccggagcctcgtgttccccgaggccgagaatagaaagtggaccattatggccgtgatggtgtcactcctcaccgactacagcccgcagcttcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 55 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatctccgcatcctccttaacaacgccgcgtttagaaacggacataacttcatggtccggaacttcagatgtggacagccgcttcaaaacaaggtccagctgaagggtcgggatcttctgaccctgaagaactttaccggagaagagatcaagtacatgctctggctctccgcggacttgaagttccgcattaagcagaagggagaatacctcccgctgcttcaaggaaagagcctcggaatgatttttgagaagcgctcaaccaggacccgcctttctactgaaactggattcgcgctgctgggtggacacccctgcttcctgacgacccaggacatccacctcggagtgaacgaatccctcactgataccgcccgggtgttatcgagcatggcagatgccgtgctggccagggtgtacaaacagtccgatctggacactctggccaaggaggcgtcaattcctatcatcaacggacttagtgacctctaccatccgattcaaatcctggccgactacctcaccctgcaagaacactacagctccctgaagggtctgacattgtcctggatcggagatggaaacaacattctccactccatcatgatgtccgccgcaaaattcggaatgcatcttcaagccgccacgcctaagggttacgaacccgacgcttccgtgactaagctcgccgagcagtacgctaaggagaacggtaccaagcttctcctgaccaacgacccactagaagcagcccacggtggaaacgtgcttattactgacacttggatctccatgggacaggaggaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccgcggagcctcgtgttccccgaggccgagaatagaaagtggaccatcatggccgtgatggtgtcactgctcaccgactacagcccgcagcttcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 56 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaacctccgcattctcctcaacaacgctgccttccggaatggacataacttcatggtccggaacttcagatgcggacagccgcttcagaacaaggtccagcttaaggggagagatctccttaccctcaaaaacttcactggcgaagaaatcaagtacatgctctggcttagtgcggatctcaagttccgcatcaagcagaagggagaatacctcccgctccttcaaggaaagagcctcggcatgatttttgagaagaggtccaccagaactcgcctttcaaccgagactgggttcgccctgcttggcggtcacccctgcttcctcactacccaagacatccacctcggcgtgaacgagagccttaccgacaccgcccgcgtgctctcctcaatggccgacgctgtgctcgcccgggtgtacaagcagtccgaccttgatactctcgccaaggaggcctccatcccaattatcaacgggctctctgatctctaccaccctatccaaatcctcgcggactacctcaccctccaagagcactatagctcgctcaagggcctcaccctttcctggattggcgacggcaacaacattcttcactcgatcatgatgtccgccgccaagttcggcatgcatctccaagccgcgacccccaagggctacgagcctgacgcatccgtgaccaagctcgccgagcagtacgcgaaggaaaatggcaccaagcttcttctcaccaacgacccccttgaggccgctcatggcggcaacgtgctcatcactgacacttggatcagcatgggccaggaggaggaaaagaagaagcgccttcaggcattccagggttaccaggtcaccatgaaaaccgccaaagtggccgcctccgactggacctttcttcactgtctcccgcggaagcctgaagaagtggatgacgaagtgttttactcccctcggtcactcgtgttcccggaagcagaaaacaggaagtggaccattatggcggtcatggtgtccctcctcaccgactacagcccgcagcttcagaaacccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 57 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatctccgcatcctccttaacaacgcagcgtttagaaacggtcacaacttcatggtccggaacttccgctgtggacagccgcttcaaaacaaggtccagctgaagggtcgggaccttctgaccctgaagaactttactggagaagagatcaagtacatgctttggctgtccgcggacttgaagttccgcattaagcagaagggagaataccttccgctgctccaaggaaagagcctgggaatgatctttgagaagcgctcaaccaggacccgcctttctactgaaactggattcgcgctgctgggtggtcacccttgcttcctgacgacccaggacattcacctcggagtgaacgagtccctcactgataccgccagagtgttatcgagcatggcagatgccgtgctggctagggtgtacaaacagtccgatctggacaccctggccaaggaggcatcaattcctattatcaacggacttagtgacctctaccatccgattcaaatcctggccgattacctcaccctgcaagaacactacagctccctgaagggtctgacattgtcctggatcggagatggaaacaacattctccattccatcatgatgtccgcggccaagttcggaatgcatctccaagccgccacgccgaaaggatacgagccggacgcttccgtgactaagctcgccgagcagtacgctaaggagaacggaaccaagcttctgctgactaacgacccgctagaagccgcccacggtggaaacgtgcttattactgacacctggatctccatgggacaggaagaagagaaaaagaagcggctgcaggcgttccagggatatcaggtcaccatgaaaaccgccaaggtcgccgcctccgactggaccttccttcactgcctgcctcggaagcctgaagaagtggacgacgaggtgttctactcgccgcggagcctcgtgttccctgaggccgagaatagaaagtggaccatcatggccgtgatggtgtcactcctcaccgactacagcccgcagcttcagaagcctaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 58 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatctccgcattctcctcaacaacgcagcctttagaaacggccacaacttcatggtccggaacttcagatgtggccagccgcttcagaacaaggtccagctcaagggccgggacctcctcaccctcaaaaactttaccggcgaagagatcaagtacatgctctggctttcggccgaccttaagttccgcatcaagcagaagggggaataccttccgctgcttcaaggaaagtccctcggcatgatctttgaaaagcgctcgaccaggacccgcctttccactgaaaccgggttcgcgcttctcggtggccacccctgcttcctcaccacccaagacattcacctcggagtgaacgaatcccttaccgataccgcaagagtgctttcgtcgatggccgatgccgtgcttgcgcgggtgtacaagcagtcagatctcgacactctcgccaaggaggcgtccattcctattatcaacggcctttccgacctttaccacccgattcagatcctcgccgattacctcaccctgcaagagcactactcgtcactcaagggtcttaccctctcctggatcggcgacggaaacaacatcctccattcgatcatgatgtccgccgccaaattcggcatgcacctccaagccgcgaccccgaagggttacgagcccgacgcttccgtgaccaagctcgccgaacagtacgctaaggaaaacggcaccaagctcctcctcactaacgaccctctcgaagcagcccatgggggcaacgtgctcattactgacacttggatctcgatgggccaggaagaggagaaaaagaagcggcttcaggcgttccagggatatcaggtcaccatgaaaaccgccaaggtcgctgcctcggactggaccttccttcactgccttccgcgcaagcctgaagaggtggacgatgaggtgttctactccccacggtcccttgtgttccccgaggccgagaataggaagtggaccatcatggccgtgatggtgtcgctcctcactgactactccccgcaacttcagaagcctaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 59 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgtttaatctgagaatacttctaaacaacgccgccttccggaatggccataactttatggttcggaatttccgctgcggccagccgctgcagaacaaggtccagctgaagggaagagacttgctgaccctcaagaacttcaccggagaagaaatcaagtatatgctgtggctgtccgccgacctgaaattccgcatcaagcagaagggcgaatatctgccgctgttgcaagggaagtccctggggatgatcttcgagaagaggtccaccagaacacggctttcaaccgaaaccgggtttgcactgctgggtggacacccctgttttctgaccactcaagatatccacctgggcgtgaacgagtcccttaccgacactgctagggtgttgtccagcatggccgatgccgtcctggctcgcgtgtacaagcagtccgacctggataccctggcaaaggaagcgtccattcccattatcaacgggctgtccgacctgtaccatccgattcaaatcctggcggactacctgactctgcaagagcattacagcagcttgaaggggcttactctctcgtggatcggcgacgggaacaacatcctgcactccatcatgatgtccgccgccaagttcgggatgcatttgcaagctgcgaccccgaaaggttacgagcccgatgctagcgtaactaagcttgccgaacagtacgccaaagagaatggtacaaaactgcttctgactaacgacccgctggaagcagcccacggcgggaacgtgctgataaccgacacctggatttcaatggggcaggaggaagagaagaagaagcgactgcaggcgttccaaggctatcaggttaccatgaaaaccgccaaagtggcagccagcgattggactttcctgcactgtctgccgcggaagcccgaggaagttgatgacgaagtattctactcaccccggagcctcgtgttccccgaggccgaaaaccggaagtggactattatggccgtgatggtgtcgctgttgaccgactacagcccgcaactgcagaagccgaagttttagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 60 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaacctgaggatccttttgaacaacgccgcctttcgcaacggccacaactttatggtccgcaatttccgctgcgggcagccgctgcagaacaaggtccagctgaagggccgggatctgctgaccctgaagaacttcaccggggaggaaatcaagtacatgctttggctctccgccgatctgaagttcagaatcaagcagaagggagagtacctcccgttgctgcaaggaaagtcactcggaatgattttcgaaaagagaagcactaggacccgcctctcaactgaaaccgggttcgcgctgctcgggggccatccgtgtttcctgactacccaagacatccacctgggagtgaacgagtcgctgaccgacaccgcacgcgtgctgtcatccatggcggacgcagtgcttgcccgggtgtacaagcagtcggacctggacactcttgccaaggaggcatcaatccccatcattaacggactgtccgatctctaccacccgattcagatcctggctgactacctaaccctgcaagagcactactcaagcctgaaggggctgaccctgtcgtggatcggggacggcaacaacattctgcactccatcatgatgtcggcggctaagttcgggatgcatttgcaagcggcaactccgaagggttatgaacccgacgcctccgtgaccaagctggccgaacagtacgccaaggaaaacggaaccaagttgctgctgactaatgatcccctggaggcggcccacggggggaacgtgctgataaccgatacctggatctccatggggcaggaagaagagaagaaaaagcggctgcaggcattccagggataccaggtcaccatgaaaaccgcaaaagtggcagccagcgactggactttcctccattgcctgccgcgaaagccggaggaggtcgatgacgaggtgttctactccccgcggtcgctggtgttcccggaggcggaaaaccggaagtggaccattatggccgtgatggtgtcactcctgactgactacagcccgcaactgcagaagccgaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 61 - RNA, 1496 nt, synthetic construct
  source 1..1496: mol_type = other RNA
cttaagggggcgctgcctacggaggtggcagccatctccttctcggcatcaagcttaccatggtgccccaggccctgctcttggtcccgctgctggtgttccccctctgcttcggcaagttccccatctacaccatccccgacaagctggggccgtggagccccatcgacatccaccacctgtcctgccccaacaacctcgtggtcgaggacgagggctgcaccaacctgagcgggttctcctacatgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccattctagatctag

SEQ ID NO: 62 - RNA, 1235 nt, synthetic construct
  source 1..1235: mol_type = other RNA
attattacatcaaaacaaaaagccgccaatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgc

SEQ ID NO: 63 - RNA, 1495 nt, synthetic construct
  source 1..1495: mol_type = other RNA
cttaagggggcgctgcctacggaggtggcagccatctccttctcggcatcaagcttaccatggtgccccaggccctgctcttggtcccgctgctggtgttccccctctgcttcggcaagttccccatctacaccatccccgacaagctggggccgtggagccccatcgacatccaccacctgtcctgccccaacaacctcgtggtcgaggacgagggctgcaccaacctgagcgggttctcctacatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaataagtgatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccattctagatctag

SEQ ID NO: 64 - RNA, 1235 nt, synthetic construct
  source 1..1235: mol_type = other RNA
attattacatcaaaacaaaaagccgccaatgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgc

SEQ ID NO: 65 - RNA, 1495 nt, synthetic construct
  source 1..1495: mol_type = other RNA
cttaagggggcgctgcctacggaggtggcagccatctccttctcggcatcaagcttaccatggtgccccaggccctgctcttggtcccgctgctggtgttccccctctgcttcggcaagttccccatctacaccatccccgacaagctggggccgtggagccccatcgacatccaccacctgtcctgccccaacaacctcgtggtcgaggacgagggctgcaccaacctgagcgggttctcctacatgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcatccgtgaccaagttggcggaacagtacgcgaaggagaacggaaccaagctcctgctgactaacgacccgctcgaggctgcgcatgggggtaacgtgctgattacggacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctagataagtgatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccattctagatctag

SEQ ID NO: 66 - RNA, 1475 nt, synthetic construct
  source 1..1475: mol_type = other RNA
tgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagtgttgggttgcgatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaataagtgatagagcggcaaaccctagctacactccatagctagtttcttttttttttgtttttttttttttttttttttttttttttttttttttttcctttcttttccttctttttttcctcttttcttggtggctccatcttagccctagtcacggctagctgtgaaaggtccgtgagccgcatgactgcagagagtgccgtaactggcctctctgcagatcatgttctag

SEQ ID NO: 67 - RNA, 1202 nt, synthetic construct
  source 1..1202: mol_type = other RNA
attattacatcaaaacaaaaagccgccaatgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctaggctggagcctcggtagccgttcctcctgcccgctgggcctcccaacgggccctcctcccctccttgcaccggcccttcctggtctttgaataaagtctgagtgggcagc

SEQ ID NO: 68 - RNA, 1202 nt, synthetic construct
  source 1..1202: mol_type = other RNA
attattacatcaaaacaaaaagccgccaatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgagctggagcctcggtagccgttcctcctgcccgctgggcctcccaacgggccctcctcccctccttgcaccggcccttcctggtctttgaataaagtctgagtgggcagc

SEQ ID NO: 69 - RNA, 1202 nt, synthetic construct
  source 1..1202: mol_type = other RNA
attattacatcaaaacaaaaagccgccaatgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcatccgtgaccaagttggcggaacagtacgcgaaggagaacggaaccaagctcctgctgactaacgacccgctcgaggctgcgcatgggggtaacgtgctgattacggacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctaggctggagcctcggtagccgttcctcctgcccgctgggcctcccaacgggccctcctcccctccttgcaccggcccttcctggtctttgaataaagtctgagtgggcagc

SEQ ID NO: 70 - RNA, 1334 nt, synthetic construct
  source 1..1334: mol_type = other RNA
aattattggttaaagaagtatattagtgctaatttccctccgtttgtcctagcttttctcttctgtcaaccccacacgcctttggcacaatgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagctcgagacacatcacaaccacaaccttctcaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctatctag

SEQ ID NO: 71 - RNA, 1334 nt, synthetic construct
  source 1..1334: mol_type = other RNA
aattattggttaaagaagtatattagtgctaatttccctccgtttgtcctagcttttctcttctgtcaaccccacacgcctttggcacaatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgactcgagacacatcacaaccacaaccttctcaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctatctag

SEQ ID NO: 72 - RNA, 1334 nt, synthetic construct
  source 1..1334: mol_type = other RNA
aattattggttaaagaagtatattagtgctaatttccctccgtttgtcctagcttttctcttctgtcaaccccacacgcctttggcacaatgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcatccgtgaccaagttggcggaacagtacgcgaaggagaacggaaccaagctcctgctgactaacgacccgctcgaggctgcgcatgggggtaacgtgctgattacggacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctagctcgagacacatcacaaccacaaccttctcaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctatctag

SEQ ID NO: 73 - RNA, 1222 nt, synthetic construct
  source 1..1222: mol_type = other RNA
attattacatcaaaacaaaaagccgccaccatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 74 - RNA, 1334 nt, synthetic construct
  source 1..1334: mol_type = other RNA
aattattggttaaagaagtatattagtgctaatttccctccgtttgtcctagcttttctcttctgtcaaccccacacgcctttggcacaatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgactcgagacacatcacaaccacaaccttctcaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctatctag

SEQ ID NO: 75 - RNA, 1235 nt, synthetic construct
  source 1..1235: mol_type = other RNA
attattacatcaaaacaaaaagccgccaatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgc

SEQ ID NO: 76 - RNA, 1496 nt, synthetic construct
  source 1..1496: mol_type = other RNA
cttaagggggcgctgcctacggaggtggcagccatctccttctcggcatcaagcttaccatggtgccccaggccctgctcttggtcccgctgctggtgttccccctctgcttcggcaagttccccatctacaccatccccgacaagctggggccgtggagccccatcgacatccaccacctgtcctgccccaacaacctcgtggtcgaggacgagggctgcaccaacctgagcgggttctcctacatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaataagtgaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccattctagatctag

SEQ ID NO: 77 - RNA, 1380 nt, synthetic construct
  source 1..1380: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgggcgtcttcaacctgcggatcctgctgaacaacgccgccttccggaacggccacaacttcatggtccgcaacttcagatgcggccagcccctgcagaacaaggtgcagctgaagggccgggacctgctgaccctgaagaacttcaccggcgaagagatcaagtacatgctgtggctgagcgccgacctgaagttccggatcaagcagaagggcgagtacctgcccctgctgcaaggcaagagcctgggcatgatcttcgagaagcggagcacccggacccggctgagcaccgagacaggctttgccctgctgggaggccacccctgctttctgaccacccaggacatccacctgggcgtgaacgagagcctgaccgacaccgccagagtgctgagcagcatggccgacgccgtgctggcccgggtgtacaagcagagcgacctggacaccctggccaaagaggccagcatccccatcatcaacggcctgagcgacctgtaccaccccatccagatcctggccgactacctgaccctgcaggaacactacagctccctgaagggcctgaccctgagctggatcggcgacggcaacaacatcctgcacagcatcatgatgagcgccgccaagttcggcatgcatctgcaggccgccacccccaagggctacgagcctgatgccagcgtgaccaagctggccgagcagtacgccaaagagaacggcaccaagctgctgctgaccaacgaccccctggaagccgcccacggcggcaacgtgctgatcaccgacacctggatcagcatgggccaggaagaggaaaagaagaagcggctgcaggccttccagggctaccaggtcacaatgaagaccgccaaggtggccgccagcgactggaccttcctgcactgcctgccccggaagcccgaagaggtggacgacgaggtgttctacagcccccggtccctggtgttccccgaggccgagaaccggaagtggaccattatggccgtgatggtgtccctgctgaccgactactccccccagctgcagaagcccaagttctagataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 78 - RNA, 1380 nt, synthetic construct
  source 1..1380: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgggcgtcttcaacctgcggatcctgctgaacaacgccgccttccggaacggccacaacttcatggtccgcaacttcagatgcggccagcccctgcagaacagggtgcagctgaagggccgggacctgctgaccctgaagaacttcaccggcgaagagatcaggtacatgctgtggctgagcgccgacctgaagttccggatcaagcagaagggcgagtacctgcccctgctgcaaggcaagagcctgggcatgatcttcgagaagcggagcacccggacccggctgagcaccgagacaggctttgccctgctgggaggccacccctgctttctgaccacccaggacatccacctgggcgtgaacgagagcctgaccgacaccgccagagtgctgagcagcatggccgacgccgtgctggcccgggtgtacaagcagagcgacctggacaccctggccaaagaggccagcatccccatcatcaacggcctgagcgacctgtaccaccccatccagatcctggccgactacctgaccctgcaggaacactacagctccctgaagggcctgaccctgagctggatcggcgacggcaacaacatcctgcacagcatcatgatgagcgccgccaagttcggcatgcatctgcaggccgccacccccaagggctacgagcctgatgccagcgtgaccaagctggccgagcagtacgccaaagagaacggcaccaagctgctgctgaccaacgaccccctggaagccgcccacggcggcaacgtgctgatcaccgacacctggatcagcatgggccaggaagaggaaaagaagaagcggctgcaggccttccagggctaccaggtcacaatgaagaccgccaaggtggccgccagcgactggaccttcctgcactgcctgccccggaagcccgaagaggtggacgacgaggtgttctacagcccccggtccctggtgttccccgaggccgagaaccggaagtggaccattatggccgtgatggtgtccctgctgaccgactactccccccagctgcagaagcccaagttctagataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 79 - RNA, 1380 nt, synthetic construct
  source 1..1380: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctggtcttcaacctgcggatcctgctgaacaacgccgccttccggaacggccacaacttcatggtccgcaacttcagatgcggccagcccctgcagaacagggtgcagctgaagggccgggacctgctgaccctgaagaacttcaccggcgaagagatcaggtacatgctgtggctgagcgccgacctgaagttccggatcaagcagaagggcgagtacctgcccctgctgcaaggcaagagcctgggcatgatcttcgagaagcggagcacccggacccggctgagcaccgagacaggctttgccctgctgggaggccacccctgctttctgaccacccaggacatccacctgggcgtgaacgagagcctgaccgacaccgccagagtgctgagcagcatggccgacgccgtgctggcccgggtgtacaagcagagcgacctggacaccctggccaaagaggccagcatccccatcatcaacggcctgagcgacctgtaccaccccatccagatcctggccgactacctgaccctgcaggaacactacagctccctgaagggcctgaccctgagctggatcggcgacggcaacaacatcctgcacagcatcatgatgagcgccgccaagttcggcatgcatctgcaggccgccacccccaagggctacgagcctgatgccagcgtgaccaagctggccgagcagtacgccaaagagaacggcaccaagctgctgctgaccaacgaccccctggaagccgcccacggcggcaacgtgctgatcaccgacacctggatcagcatgggccaggaagaggaaaagaagaagcggctgcaggccttccagggctaccaggtcacaatgaagaccgccaaggtggccgccagcgactggaccttcctgcactgcctgccccggaagcccgaagaggtggacgacgaggtgttctacagcccccggtccctggtgttccccgaggccgagaaccggaagtggaccattatggccgtgatggtgtccctgctgaccgactactccccccagctgcagaagcccaagttctagataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 80 - RNA, 1377 nt, synthetic construct
  source 1..1377: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgttcaacctgaggatcctgctgaacaacgcagctttcaggaacggccacaacttcatggtgaggaacttccggtgcggccagcccctgcagaacaaggtgcagctgaagggcagggacctgctgaccctgaagaacttcaccggagaggagatcaagtacatgctgtggctgagcgcagacctgaagttcaggatcaagcagaagggagagtacctgcccctgctgcaggggaagtccctgggcatgatcttcgagaagaggagtaccaggaccaggctgagcaccgaaaccggcttcgccctgctgggaggacacccctgcttcctgaccacccaggacatccacctgggcgtgaacgagagtctgaccgacaccgccagggtgctgtctagcatggccgacgccgtgctggccagggtgtacaagcagtcagacctggacaccctggctaaggaggccagcatccccatcatcaacggcctgagcgacctgtaccaccccatccagatcctggctgactacctgaccctgcaggagcactacagctctctgaagggcctgaccctgagctggatcggcgacgggaacaacatcctgcacagcatcatgatgagcgccgccaagttcggcatgcacctgcaggccgctacccccaagggttacgagcccgacgccagcgtgaccaagctggcagagcagtacgccaaggagaacggcaccaagctgctgctgaccaacgaccccctggaggccgcccacggaggcaacgtgctgatcaccgacacctggatcagcatgggacaggaggaggagaagaagaagcggctgcaggctttccagggttaccaggtgaccatgaagaccgccaaggtggctgccagcgactggaccttcctgcactgcctgcccaggaagcccgaggaggtggacgacgaggtgttctactctcccaggagcctggtgttccccgaggccgagaacaggaagtggaccatcatggctgtgatggtgtccctgctgaccgactacagcccccagctgcagaagcccaagttctgaataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 81 - RNA, 1377 nt, synthetic construct
  source 1..1377: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgttcaacctgaggatcctgctgaacaacgcagctttcaggaacggccacaacttcatggtgaggaacttccggtgcggccagcccctgcagaacaaggtgcagctgaagggcagggacctgctgaccctgaagaacttcaccggagaggagatcaagtacatgctgtggctgagcgcagacctgaagttcaggatcaagcagaagggagagtacctgcccctgctgcaggggaagtccctgggcatgatcttcgagaagaggagtaccaggaccaggctgagcaccgaaaccggcttcgccctgctgggaggacacccctgcttcctgacgacccaggacatccacctcggagtgaacgaatccctcaccgataccgcccgggtgttatcaagcatggcagatgccgtgctggccagggtgtacaaacagtccgatctggacactctggctaaggaggccagcatccccatcatcaacggcctgagcgacctgtaccaccccatccagatcctggctgactacctgaccctgcaggagcactacagctctctgaagggcctgaccctgagctggatcggcgacgggaacaacatcctgcactccatcatgatgtccgccgcgaagttcggaatgcatctgcaagccgccacgccaaaaggatacgaaccggatgcgcccgtgacaaagttggcggaacagtacgctaaggagaacggaaccaagctgctgctgaccaacgaccccctggaggccgcccacggaggcaacgtgctgatcaccgacacctggatcagcatgggacaggaggaggagaagaagaagcggctgcaggctttccagggttaccaggtgaccatgaagaccgccaaggtggctgccagcgactggaccttcctgcactgcctgcccaggaagcccgaggaggtggacgacgaggtgttctactctcccaggagcctggtgttccccgaggccgagaacaggaagtggaccatcatggctgtgatggtgtccctgctgaccgactacagcccccagctgcagaagcccaagttctgaataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 82 - RNA, 1377 nt, synthetic construct
  source 1..1377: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 83 - RNA, 1359 nt, synthetic construct
  source 1..1359: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgactagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 84 - RNA, 1371 nt, synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaacttgagaatcctgctgaacaacgccgcctttcgcaacggtcacaattttatggtcagaaacttcagatgcggacagcccctccaaaacaaggtccagctgaagggccgcgatctcctcaccctgaagaacttcacgggggaggagatcaagtacatgctgtggctctccgctgacctgaagttcaggatcaagcagaagggagaatatctgccgctgctgcaagggaagtccctggggatgattttcgagaagcggagcacccggactcggctctccactgaaactggtttcgcccttctgggcggtcacccctgcttcctgaccactcaagacattcacctcggagtgaacgagtccttgactgacaccgcccgggtgctgtcgagcatggcagacgccgtgctagcccgcgtgtacaagcagtcagacctcgataccctggccaaggaggcttcgatcccgatcatcaacgggttgtccgacctgtaccacccgattcagattctcgccgactacctcaccctgcaagagcattacagctccctgaaggggcttaccctgtcctggattggcgacggaaacaacatcctgcactccattatgatgtcggcggccaagttcggcatgcacctccaagccgcgacccctaagggttacgaaccagacgcgtcagtgactaagctggccgaacagtacgcaaaggaaaatggcacgaagctgctcctgaccaacgatccgttggaagccgcccatggcggaaatgtgctcatcaccgacacctggatctcgatgggacaggaggaagagaagaagaagcggctgcaggcgttccagggctaccaggtcaccatgaaaactgccaaggtggccgccagcgactggaccttcctgcactgccttccgcgcaagcctgaggaggtggacgatgaagtgttctactctccacggtccctggtgttccccgaggcggagaaccgcaaatggaccatcatggctgtgatggtcagcctgctgaccgattacagccctcagttgcaaaagccgaagttttgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 85 - RNA, 1371 nt, synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgttcaacctccgcatcctcctcaacaacgccgcattcagaaacgggcacaacttcatggtcagaaacttccgctgcgggcaacccctacaaaacaaggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 86 - RNA, 1771 nt, synthetic construct
  source 1..1771: mol_type = other RNA
ctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataatatgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctgaataagtagatagtgcagtcactggcacaacgcgttgcccggtaagccaatcgggtatacacggtcgtcatactgcagacagggttcttctactttgcaagatagtctagagtagtaaaataaatagtataagtctag

SEQ ID NO: 87 - RNA, 1771 nt, synthetic construct
  source 1..1771: mol_type = other RNA
ctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataatatgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcatccgtgaccaagttggcggaacagtacgcgaaggagaacggaaccaagctcctgctgactaacgacccgctcgaggctgcgcatgggggtaacgtgctgattacggacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctgaataagtagatagtgcagtcactggcacaacgcgttgcccggtaagccaatcgggtatacacggtcgtcatactgcagacagggttcttctactttgcaagatagtctagagtagtaaaataaatagtataagtctag

SEQ ID NO: 88 - RNA, 1771 nt, synthetic construct
  source 1..1771: mol_type = other RNA
ctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataatatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaataagtagatagtgcagtcactggcacaacgcgttgcccggtaagccaatcgggtatacacggtcgtcatactgcagacagggttcttctactttgcaagatagtctagagtagtaaaataaatagtataagtctag

SEQ ID NO: 89 - RNA, 1368 nt, synthetic construct; identical to SEQ ID NO: 34
  source 1..1368: mol_type = other RNA

SEQ ID NO: 90 - RNA, 1368 nt, synthetic construct; identical to SEQ ID NO: 34
  source 1..1368: mol_type = other RNA

SEQ ID NO: 91 - RNA, 1368 nt, synthetic construct; identical to SEQ ID NO: 34
  source 1..1368: mol_type = other RNA

SEQ ID NO: 92 - RNA, 1368 nt, synthetic construct; identical to SEQ ID NO: 34
  source 1..1368: mol_type = other RNA

SEQ ID NO: 93 - RNA, 1368 nt, synthetic construct; identical to SEQ ID NO: 34
  source 1..1368: mol_type = other RNA

SEQ ID NO: 94 - RNA, 1371 nt, synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatggtgttcaacctccgcatcctcctcaacaacgccgcattcagaaacgggcacaacttcatggtcagaaacttccgctgcgggcaacccctacaaaacaaggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 95 - RNA, 1371 nt, synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatggtgttcaacctccgcatcctcctcaacaacgccgcattcagaaacgggcacaacttcatggtcagaaacttccgctgcgggcaacccctacaaaaccgggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 96 - RNA, 1371 nt, synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatggtgttcaacctccgcatcctcctcaacaacgccgcattcagaaacgggcacaacttcatggtcagaaacttccgctgcgggcaacccctacaaaaccgggtccagctcaaggggcgggacctcctgaccctgaagaacttcaccggcgaagagatccggtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 97 - RNA, 1371 nt, synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctggtcaacctccgcatcctcctcaacaacgccgcattcagaaacgggcacaacttcatggtcagaaacttccgctgcgggcaacccctacaaaacaaggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 98 - RNA, 1371 nt, synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctggtcaacctccgcatcctcctcaacaacgccgcattcagaaacgggcacaacttcatggtcagaaacttccgctgcgggcaacccctacaaaaccgggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 99 - RNA, 1371 nt, synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctggtcaacctccgcatcctcctcaacaacgccgcattcagaaacgggcacaacttcatggtcagaaacttccgctgcgggcaacccctacaaaaccgggtccagctcaaggggcgggacctcctgaccctgaagaacttcaccggcgaagagatccggtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 100 - RNA, 1371 nt, synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctggtcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 101 - RNA, 1371 nt, synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctggtcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 102 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttgtcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 103 - RNA, 1368 nt, synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttgtcaatctccgcatcctccttaacaacgccgcgtttagaaacggccacaacttcatggtccggaacttcagatgtggccagccgcttcaaaaccgggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 104 - RNA, 1371 nt, synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgggccttgtcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 105 - RNA, 1371 nt, synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgggccttgtcaatctccgcatcctccttaacaacgccgcgtttagaaacggccacaacttcatggtccggaacttcagatgtggccagccgcttcaaaaccgggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 106 - RNA, 1374 nt, synthetic construct
  source 1..1374: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgggcggacttgtcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 107 - RNA, 1377 nt, synthetic construct
  source 1..1377: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatggcccttttcaatctccgcatcctccttaacaacgccgcgtttagaaacggccacaacttcatggtccggaacttcagatgtggccagccgcttcaaggcaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 108 - RNA, 1377 nt, synthetic construct
  source 1..1377: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatggcccttttcaatctccgcatcctccttaacaacgccgcgtttagaaacggccacaacttcatggtccggaacttcagatgtggccagccgcttcaaggccgggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 109 - RNA, 1377 nt, synthetic construct
  source 1..1377: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatggcccttttcaatctccgcatcctccttaacaacgccgcgtttagaaacggccacaacttcatggtccggaacttcagatgtggccagccgcttcaaggccgggtccagctgaagggccgggatcttctgaccctgaagaactttactggcgaagagatcaggtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 110 - RNA, 1377 nt, synthetic construct
  source 1..1377: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatggcccttgtcaatctccgcatcctccttaacaacgccgcgtttagaaacggccacaacttcatggtccggaacttcagatgtggccagccgcttcaaggcagggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctaagtgaatagactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 111 - RNA, 1377 nt, synthetic construct
  source 1..1377: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatggcccttttcaatctccgcatcctccttaacaacgccgcgtttagaaacggccacaacttcatggtccggaacttcagatgtggccagccgcttcaagtcaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 112 - RNA, 1377 nt, synthetic construct
  source 1..1377: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatggcccttttcaatctccgcatcctccttaacaacgccgcgtttagaaacggccacaacttcatggtccggaacttcagatgtggccagccgcttcaagtcagggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 113 - RNA, 1282 nt, synthetic construct
  source 1..1282: mol_type = other RNA
ggcagaaaaatttgctacattgtttcacaaacttcaaatattattcatttatttagatctattattacatcaaaacaaaaagccgccaccatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 114 - RNA, 1285 nt, synthetic construct
  source 1..1285: mol_type = other RNA
ggcagaaaaatttgctacattgtttcacaaacttcaaatattattcatttatttagatctattattacatcaaaacaaaaagccgccaccatgggagtattcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 115 - RNA, 1285 nt, synthetic construct
  source 1..1285: mol_type = other RNA
ggcagaaaaatttgctacattgtttcacaaacttcaaatattattcatttatttagatctattattacatcaaaacaaaaagccgccaccatgggagtattcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 116 - RNA, 1285 nt, synthetic construct
  source 1..1285: mol_type = other RNA
ggcagaaaaatttgctacattgtttcacaaacttcaaatattattcatttatttagatctattattacatcaaaacaaaaagccgccaccatgctggtattcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 117 - RNA, 1225 nt, synthetic construct
  source 1..1225: mol_type = other RNA
attattacatcaaaacaaaaagccgccaccatgggagtattcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 118 - RNA, 1225 nt, synthetic construct
  source 1..1225: mol_type = other RNA
attattacatcaaaacaaaaagccgccaccatgggagtattcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 119 - RNA, 1228 nt, synthetic construct
  source 1..1228: mol_type = other RNA
attattacatcaaaacaaaaagccgccaccatgctggtattcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcagcatctag

SEQ ID NO: 120 - RNA, 1376 nt, synthetic construct
  source 1..1376: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgttgttcaacttgaggatcttgttgaacaacgccgccttcaggaacggacacaacttcatggtaaggaacttcaggtgcggacagcccttgcagaacaaagtacagttgaaaggaagggacttgttgacattgaaaaacttcacaggagaagaaatcaaatacatgttgtggttgtcggccgacttgaaattcaggatcaaacagaaaggagaatacttgcccttgttgcagggaaaatcgttgggaatgatcttcgaaaaaaggtcgacaaggacaaggttgtcgacagaaacaggattcgccttgttgggaggacacccctgcttcttgacaacacaggacatccacttgggagtaaacgaatcgttgacagacacagccagggtattgtcgtcgatggccgacgccgtattggccagggtatacaaacagtcggacttggacacattggccaaagaagcctcgatccccatcatcaacggattgtcggacttgtaccaccccatccagatcttggccgactacttgacattgcaggaacactactcgtcgttgaaaggattgacattgtcgtggatcggagacggaaacaacatcttgcactcgatcatgatgtcggccgccaaattcggaatgcacttgcaggccgccacacccaaaggatacgaacccgacgcctcggtaacaaaattggccgaacagtacgccaaagaaaacggaacaaaattgttgttgacaaacgaccccttggaagccgcccacggaggaaacgtattgatcacagacacatggatctcgatgggacaggaagaagaaaaaaaaaaaaggttgcaggccttccagggataccaggtaacaatgaaaacagccaaagtagccgcctcggactggacattcttgcactgcttgcccaggaaacccgaagaagtagacgacgaagtattctactcgcccaggtcgttggtattccccgaagccgaaaacaggaaatggacaatcatggccgtaatggtatcgttgttgacagactactcgccccagttgcagaaacccaaattctgaatagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 121 - RNA, 1222 nt, synthetic construct
  source 1..1222: mol_type = other RNA
attattacatcaaaacaaaaagccgccaccatgctgttcaacctgcgcatcctgctgaacaacgccgccttccgcaacggccacaacttcatggtgcgcaacttccgctgcggccagcccctgcagggcaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 122 - RNA, 1222 nt, synthetic construct
  source 1..1222: mol_type = other RNA
attattacatcaaaacaaaaagccgccaccatgctgttcaacctgcgcatcctgctgaacaacgccgccttccgcaacggccacaacttcatggtgcgcaacttccgctgcggccagcccctgcagggccgggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 123 - RNA, 1222 nt, synthetic construct
  source 1..1222: mol_type = other RNA
attattacatcaaaacaaaaagccgccaccatgctgttcaacctgcgcatcctgctgaacaacgccgccttccgcaacggccacaacttcatggtgcgcaacttccgctgcggccagcccctgcagggccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 124 - RNA, 1282 nt, synthetic construct
  source 1..1282: mol_type = other RNA
ggcagaaaaatttgctacattgtttcacaaacttcaaatattattcatttatttagatctattattacatcaaaacaaaaagccgccaccatgctgttcaacctgcgcatcctgctgaacaacgccgccttccgcaacggccacaacttcatggtgcgcaacttccgctgcggccagcccctgcagggcaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 125 - RNA, 21 nt, synthetic construct
  source 1..21: mol_type = other RNA
cacaaagagtaaagaagaaca

SEQ ID NO: 126 - RNA, 21 nt, synthetic construct
  source 1..21: mol_type = other RNA
aacactaaaagtagaagaaaa

SEQ ID NO: 127 - RNA, 21 nt, synthetic construct
  source 1..21: mol_type = other RNA
ctcagaaagataagatcagcc

SEQ ID NO: 128 - DNA, 1065 bp, synthetic construct; identical to SEQ ID NO: 2
  source 1..1065: mol_type = other DNA

SEQ ID NO: 129 - DNA, 1065 bp, synthetic construct
  source 1..1065: mol_type = other DNA
atgctctttaatctgcgcatcttactgaacaacgccgcattccggaacggtcacaacttcatggtccgcaatttccgctgtggccagccgcttcaaaacaaggtccagctgaagggacgggatctgctgacactgaagaacttcaccggagaagagatcaagtacatgctgtggctcagcgcagacttgaagttccggatcaagcagaagggagaatacttgcccctgctgcaaggaaagtcgctgggaatgatttttgagaagcggtcaactcgcaccagactctccaccgaaactggtttcgcactgcttggcgggcacccttgcttcctgacgactcaggacatccacctcggcgtgaacgaatcgctaaccgataccgccagagtgctttcttccatggccgacgcggtgctggccagggtgtacaagcagtccgacctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcgtccgtgaccaagttggcggaacagtacgcgaaggagaacggaaccaagcttctgctgactaacgaccccctcgaggctgcgcatgggggcaacgtgctgattaccgacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctag

SEQ ID NO: 130 - DNA, 1065 bp, synthetic construct
  source 1..1065: mol_type = other DNA
atgctgtttaacctacgtattttgctcaacaatgcagcctttagaaacggacataactttatggttcgaaactttcgctgcgggcagccactgcagaacaaggtccagctgaaagggagagatttgctcacgctgaagaactttactggcgaagaaatcaagtatatgctgtggttgtccgcggacctcaagtttcggattaagcagaaaggggagtatctgccactgctgcaaggaaagagcctcggcatgatcttcgagaagcggagcactcggaccaggctgagtaccgaaactggcttcgcattgttgggtggacatccatgttttctgacaacgcaggacattcatctgggcgtgaacgagagtctgacggacacagctcgcgttctgtcctctatggctgatgcggtgttggcccgggtctataagcagtccgatttggacaccttggctaaggaagctagcataccgattatcaatgggctgtccgacctgtatcaccctattcaaatcctggccgactacctcacactgcaagaacactatagctcattgaagggactgaccctgagctggataggggacggaaacaacatcctacatagcattatgatgtccgctgccaagtttggcatgcatcttcaagccgccacgccaaagggttatgagcccgacgcgtcagtgacaaagctggccgagcagtacgctaaggagaatggtaccaaattactgctgactaatgatccactggaggctgcacatggcggcaatgtactgatcaccgacacatggatctcgatgggccaggaggaagaaaagaagaagaggcttcaggccttccaaggctaccaggtcaccatgaaaacagctaaggttgcagcatctgattggacctttctgcactgtctgccaaggaagcccgaagaggtggacgatgaagtattctatagcccacggagtttggtgttccctgaggctgaaaataggaagtggacaattatggccgtaatggtgtccctgttaaccgactactctccgcaactgcagaaacctaagttttag

SEQ ID NO: 131 - DNA, 1065 bp, synthetic construct
  source 1..1065: mol_type = other DNA
atgctgtttaacttaaggatcctgctgaacaacgccgcttttcgtaacggtcataactttatggtccggaactttagatgtggccagccgctgcagaacaaggttcagctgaaggggagggatctgctgaccttgaagaactttaccggcgaagagatcaagtacatgttgtggctgagcgccgatctgaagtttaggattaagcagaagggggagtatttgccactgctgcaaggaaaatccttggggatgatcttcgagaagcgctccactagaacccggctaagcacagaaaccggcttcgcacttctgggtggacatccctgttttctgacgacgcaggatatacacctgggcgtgaatgagagtctgacggacacagctagggtgttgagcagcatggccgatgcagtactggcccgcgtttataagcagagcgacttggacacactggccaaggaagcgtcaattccgattatcaatgggctgtcagacctgtatcatcccattcaaatcttggctgactatctgaccctgcaagaacattacagctccctgaagggcctcacgttgtcctggattggcgacggaaacaacattctgcattcgatcatgatgagcgctgctaagtttggcatgcacctccaagccgctacacctaagggatatgagcctgatgccagcgtaaccaagctggccgaacagtacgcgaaggagaatggcacgaaactgctgttgacaaatgacccactggaggcagctcacggtggcaacgtgctgatcaccgacacgtggatatctatgggacaggaagaagagaagaagaagcggctgcaggcattccaagggtatcaggtcaccatgaaaacggccaaggttgctgcatccgactggacatttctgcattgcttgccccgcaaaccagaagaagtagacgacgaagtcttttattccccacggtcgctggtgttccccgaggcggagaatcgaaagtggacgattatggccgtgatggtgtccctgctgactgattactctccccaactgcaaaagcctaagttttag

SEQ ID NO: 132 - DNA, 1065 bp, synthetic construct
  source 1..1065: mol_type = other DNA
atgcttttcaacctgaggatcctcctgaacaacgccgcctttcgcaatggtcacaactttatggtccggaacttcagatgcggccagccgctgcagaacaaggtccagctgaagggacgggatctgctgactctgaagaacttcaccggagaagagatcaagtacatgctgtggctgtcggccgacctgaagttcaggatcaagcagaagggagaatacctcccgctgctgcaaggaaagtccctgggcatgattttcgagaagcgctcgaccagaactcggttgtccaccgaaaccgggtttgcgctgctgggcggacatccttgcttcctgacgactcaggatattcacctgggagtgaacgagtcgctgaccgacaccgccagagtgctgagctcgatggccgacgccgtgttggcacgcgtgtacaagcagtccgatctggataccctggccaaagaagcttccatcccgatcattaacgggctgagcgacctctaccaccccattcaaatcctggccgactacctgactctgcaagaacactacagctcgctgaaggggttgactctgtcctggatcggcgacggaaacaacatcctgcactccatcatgatgtcggccgcaaagttcggcatgcatttgcaagccgccaccccaaagggctacgaaccagacgcgagcgtcaccaagctggccgaacagtacgcgaaggaaaatggtactaagctgctgctgaccaacgacccattggaagctgcccatggtggaaacgtgctgatcaccgacacctggatctcgatgggccaggaagaggagaagaagaagcggctgcaggcgttccaggggtatcaggtcaccatgaaaacagccaaagtggcagcgtcagactggaccttcctccactgtctgcctcgcaagccagaggaggtggacgacgaggtgttctactcccctcggtccctcgtgttccctgaggctgagaaccggaagtggaccattatggccgtgatggtgtcactcctgactgattactccccgcaactgcagaagcccaagttctag

SEQ ID NO: 133 - DNA, 1065 bp, synthetic construct
  source 1..1065: mol_type = other DNA
atgctgtttaacctgaggatcctattgaacaatgctgcttttcgtaatggccataactttatggttcggaactttagatgcgggcagccactgcagaacaaggtccagttgaaaggccgcgatctgttgacattgaagaactttaccggcgaagagattaagtatatgctgtggctgtctgctgacctcaagtttcgaatcaagcagaagggcgaatatctccccctgctgcaaggaaagtctctcggcatgatctttgagaagcggagtacccgaacacggctgagcaccgaaacgggcttcgcactgctggggggccatccctgttttctgacaacgcaggacatccacttgggggttaacgaatcattgactgataccgcccgcgtactgtcatccatggccgacgctgtgctggctagggtgtacaagcagtcagatctggatacactggccaaggaagctagcataccaatcatcaatggactgagtgacctttatcacccgattcaaatactagccgattatctgaccctgcaagagcattactcctcgctgaaaggcctcacgctgtcctggatcggcgacggcaacaacattctgcatagtattatgatgtctgctgccaaattcggcatgcatctgcaagctgctacgccgaagggttatgaacccgacgcgtcagttacgaagctcgctgagcagtatgcaaaggagaatggcacaaagctgttgcttaccaacgatcccctggaagctgctcatggcggcaatgtgctgattactgacacctggatttcaatgggccaggaggaggagaagaagaagaggttacaggcttttcaaggttaccaagtcacgatgaaaaccgctaaggtcgcagccagcgactggacattcctgcactgtctgccaagaaagccggaagaagtggacgacgaggtgttctattccccgcggtctttggtgtttccggaggccgaaaacaggaaatggaccattatggccgtgatggtatcgttgctgacggactacagccctcagttgcaaaagcccaagttctag

SEQ ID NO: 134 - DNA, 1065 bp, synthetic construct
  source 1..1065: mol_type = other DNA
atgctctttaacctccgcatcctcctcaacaacgccgccttccggaatgggcataacttcatggtccggaacttcagatgcggccagcccctgcaaaacaaggtccagttgaagggacgggacctccttacgctgaagaactttaccggagaagagattaagtacatgctgtggttgtccgctgacctcaagttccgcattaagcagaagggagaatatctgccgctgctgcaaggaaagagcctgggcatgatcttcgaaaagcgctccactagaacccggctgtcgactgagactggattcgccttgctcggtggacacccgtgcttcctgacgacccaggacatccacctgggagtgaacgagtcacttacggataccgcgagggtgctgtcctcaatggccgacgcagtgctcgcgcgcgtgtacaagcagtcagatctggataccctggccaaggaagccagcattcccatcatcaacggactgagcgacctttaccacccaatccagatcctcgccgactacttaaccctgcaagagcactacagctccctgaagggactgactctgtcctggatcggggatggaaacaacatcctgcactccatcatgatgtctgccgctaagtttgggatgcatctgcaagccgcaacccctaagggatacgagcccgacgcctcggtgaccaaacttgcggaacagtacgccaaggaaaacggtaccaagctgctgctgaccaacgaccctctggaagcggcccacggaggaaatgtgctgattaccgacacctggatttcgatgggccaggaggaggagaagaagaagagactgcaggcgttccagggatatcaggtcaccatgaaaaccgccaaggtcgccgccagcgactggaccttcctgcactgtctccctcggaaaccggaagaagtggatgacgaggtgttctactccccgcgctcgctggtgttcccggaggctgaaaacaggaagtggacaatcatggccgtgatggtgtccctgttgaccgactactccccacaactgcagaagcccaagttctag

SEQ ID NO: 135 - DNA, 1065 bp, synthetic construct
  source 1..1065: mol_type = other DNA
atgcttttcaatctgcgcatcctcctgaacaacgccgccttccgcaatggacacaactttatggtccgcaacttccgctgtgggcagccgctgcagaacaaggtccagctcaaggggagagatctcctgaccctgaagaacttcactggagaggagatcaagtacatgctgtggctgtccgccgacctgaaatttcggattaagcagaagggcgaatacctcccactgctgcaaggaaagtctttgggcatgatcttcgaaaagagaagcacccggacccggttgagcaccgaaactgggttcgcgctcctcggtggacacccgtgcttcctgaccacccaagatattcatctgggtgtcaacgaaagcctgaccgacaccgccagggtgctgtcatccatggctgacgcagtgctcgcccgggtgtacaagcagtcagacctggacaccctcgccaaggaagcttcgatccctatcatcaacggactttccgacctgtaccaccccatccaaattctggccgactacctgactctgcaagaacactatagctcgctgaaaggacttactctgtcctggatcggggacggcaacaacattctccattccatcatgatgtccgctgccaagttcggaatgcaccttcaagcagcgactcccaagggatacgaacctgatgcctccgtgactaagctggcagagcagtacgccaaggagaacggtacaaagctgctgctcacgaacgaccccctggaggcggcccacggcggaaacgtgctgattaccgatacctggatctcaatgggccaggaagaggagaagaagaagcggctccaggcgtttcaaggctaccaggtcaccatgaaaaccgcgaaggtcgccgcctccgactggactttcttgcactgcctgccgcggaagcccgaggaagtggatgacgaagtgttctactcgccgagatcgttggtgttccctgaggccgaaaacaggaagtggaccatcatggccgtgatggtgtccctgctgactgattacagcccacagctgcagaagcctaagttctag

SEQ ID NO: 136 - DNA, 1065 bp, synthetic construct
  source 1..1065: mol_type = other DNA
atgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctag

SEQ ID NO: 137 - DNA, 1065 bp, synthetic construct
  source 1..1065: mol_type = other DNA
atgcttttcaacctgagaatcctcctgaacaacgccgccttccgcaatggtcataacttcatggtccgcaactttcgctgcggacagcctctccaaaacaaggtccagctcaaggggcgcgacctcctcacactgaagaacttcactggagaagaaatcaagtacatgctgtggctgagcgccgatctgaagttccggatcaagcagaagggagagtaccttcctctgctgcaagggaagtccttgggaatgattttcgagaagcggtccacccggaccaggctgagcactgaaactggcttcgccctgctgggaggccacccttgtttcctgaccactcaggacatccacctgggcgtgaacgagtccctgaccgatactgccagagtgctgtcctccatggccgacgccgtgctcgcccgggtgtacaagcagtcagacctcgatacgctggccaaggaagcctccattcccattatcaatggtctgtcggacctctaccatccaatccaaatcctcgccgactacctgactctgcaagaacactacagctcactcaagggcctcaccctctcctggatcggcgacggaaacaacatccttcactcgattatgatgtcggccgcgaagttcgggatgcacctccaagctgccactccaaaaggctacgagccggatgcctcagtgactaagttggcggaacagtatgcgaaggagaacggtaccaagctcctgctgactaacgacccgctggaggccgcccacgggggaaacgtgctcatcaccgatacttggatttccatgggacaggaggaagagaagaagaagcggttgcaggcatttcagggctaccaggtcaccatgaaaactgccaaagtcgccgccagcgactggaccttcctgcactgcctgccccgcaagcctgaagaagtggacgacgaggtgttctactctccccggtccctcgtgttccctgaggccgaaaacaggaagtggaccatcatggctgtgatggtgtccctcctgaccgactacagccctcagctccaaaaacccaagttttag

SEQ ID NO: 138 - DNA, 1065 bp, synthetic construct
  source 1..1065: mol_type = other DNA
atgcttttcaacctgagaatcctcttgaacaatgctgcttttcggaatggccacaactttatggttcggaacttccgttgcggccagcctttacaaaacaaggtccagctgaagggccgggatttgctcacactgaagaactttactggggaggagattaagtatatgctgtggctgtccgctgacctgaagtttaggatcaagcagaagggcgaatatctgccgctgctgcaagggaaaagtctgggcatgatttttgaaaagcgctctacccggaccagactgtctacggaaacaggctttgccctgctgggcggccacccctgttttctgacaacgcaggacatccatctgggcgtgaacgaatcactgaccgatactgctcgggtactcagttctatggctgacgcagtgctggctagggtgtacaagcagagcgacttggacacactggctaaggaggccagcatccccattatcaatggcctgtctgatttgtaccatcccattcaaatcctggctgattatctgacactacaagagcattactcaagtctgaagggtttgactctctcctggatcggcgacggcaacaacattttacattccattatgatgagtgctgctaagtttggcatgcatttgcaagctgctaccccaaagggctatgaacctgacgctagcgtaaccaagttggccgaacagtatgctaaagagaatggcaccaagctgctcctgacgaatgaccccctggaagctgctcatggcggaaacgtacttataactgatacatggattagcatgggccaggaagaggagaagaagaagagactgcaggccttccaaggctatcaggtcaccatgaaaactgccaaggttgcagctagcgactggaccttcctgcactgtttgccgaggaaacccgaggaggtggacgatgaagtcttttattctccccgctccttggtgtttcccgaggctgaaaatcgaaagtggacgataatggcagtgatggtgtccctactgaccgactattctccacaactgcagaagcctaaattctag

SEQ ID NO: 139 - DNA, 1065 bp, synthetic construct
  source 1..1065: mol_type = other DNA
atgcttttcaatctgaggatcctgctgaacaacgctgcttttcgcaacggtcataactttatggttcgcaattttcgttgtggccagccgctgcagaacaaggttcagctgaagggcagagatctgctgactctgaagaacttcactggggaagaaatcaagtatatgttatggctgtccgcggatctgaaatttcgaatcaagcagaagggcgaatatcttcccctgctgcaagggaaatccttgggcatgatttttgagaagaggagcactaggactagattgtcaacagaaacaggctttgctttgttgggcggacatccctgctttctgacgacacaggatatccacctcggcgtaaacgagtccctcaccgacactgctagggtactgagcagcatggccgacgctgtgctagcccgggtttacaagcagtcagacctggacacccttgccaaggaagcttctattccaattatcaacggcctgagtgacctgtatcaccctattcaaatactcgccgactatttgacgcttcaagaacattacagcagcctcaagggcttaaccttgagttggataggcgacggcaacaatatcctgcattccattatgatgtctgccgctaagtttggcatgcatctacaagccgcaacacccaagggctatgaacccgacgctagcgtgaccaagctggccgagcagtatgctaaggaaaatggcacaaagctccttcttaccaacgatcccctggaggctgctcacggcggcaacgtgctgattaccgatacatggattagcatgggccaggaggaggagaaaaagaagcggctccaggcttttcaaggctatcaggtcaccatgaaaactgcaaaggtcgctgcctccgactggactttcctgcattgtctaccccgcaagcctgaggaagtggacgatgaggtgttctactccccacggagtctggtgttcccggaagcagagaatcggaagtggaccatcatggctgtcatggtgtcgctcttgactgactattctccccaactgcaaaaacccaagttttag

SEQ ID NO: 140 - DNA, 1065 bp, synthetic construct; identical to SEQ ID NO: 139
  source 1..1065: mol_type = other DNA

SEQ ID NO: 141 - DNA, 1065 bp, synthetic construct
  source 1..1065: mol_type = other DNA
atgctttttaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccacgccaaaaggatacgaaccggatgcgtccgtgacgaagttggcggaacagtacgcgaaggagaacggaaccaagcttctgctgactaacgaccccctcgaggctgcgcatgggggcaacgtgctgattaccgacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctag

SEQ ID NO: 142 - DNA, 1065 bp, synthetic construct
  source 1..1065: mol_type = other DNA
atgcttttcaacctgagaatcctcttgaacaatgctgcttttcggaatggccacaactttatggttcggaacttccgttgcggccagcctttacaaaacaaggtccagctgaagggccgggatttgctcacactgaagaactttactggagaagagatcaagtacatgctgtggctgtcggccgacctgaagttcaggatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcgtccgtgaccaagttggcggaacagtacgcgaaggagaacggaaccaagcttctgctgactaacgaccccctcgaggctgcgcatgggggcaacgtgctgattaccgacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctag

SEQ ID NO: 143 - DNA, 1065 bp, synthetic construct
  source 1..1065: mol_type = other DNA
atgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcatccgtgaccaagttggcggaacagtacgcgaaggagaacggaaccaagctcctgctgactaacgacccgctcgaggctgcgcatgggggtaacgtgctgattacggacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctag

SEQ ID NO: 144 - DNA, 1065 bp, synthetic construct
  source 1..1065: mol_type = other DNA
atgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcgtccgtgaccaagttggcggaacagtacgcgaaggagaacggaaccaagcttctgctgactaacgaccccctcgaggctgcgcatgggggcaacgtgctgattaccgacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctag

SEQ ID NO: 145 - DNA, 1065 bp, synthetic construct
  source 1..1065: mol_type = other DNA
atgctgttcaacctgcgaatcctgctgaacaacgccgcttttcggaacgggcacaactttatggtgaggaactttcgctgcggacagcccctccagaataaggtccagctgaagggcagggacctgctgaccctgaaaaatttcacaggggaggaaatcaagtatatgctgtggctgtcagctgatctgaagttccggatcaagcagaagggcgaatatctgcctctgctccagggcaaaagcctggggatgatcttcgaaaagcgcagtactcggaccagactgtcaaccgagactggattcgctctgctgggaggacacccttgttttctgaccactcaggacattcacctgggagtgaacgagtccctgaccgacactgctcgcgtcctgagctctatggccgacgctgtgctggctcgagtctacaaacagtccgacctggataccctggccaaggaagcttctatcccaattattaacggcctgtcagacctgtatcaccccatccagattctggccgattacctgaccctccaggagcactattctagtctgaaagggctgacactgagttggattggggacggaaacaatatcctgcactctattatgatgtcagccgccaagtttggaatgcacctccaggctgcaaccccaaaaggctacgaacccgatgcctcagtgacaaagctggctgaacagtacgccaaagagaacggcactaagctgctgctgaccaacgaccctctggaggccgctcacggaggcaacgtgctgatcaccgatacctggattagtatgggacaggaggaagagaagaagaagcggctccaggccttccagggctaccaggtgacaatgaaaaccgctaaggtcgcagccagcgattggacctttctgcactgcctgcccagaaagcccgaagaggtggacgacgaggtcttctactctcccagaagcctggtgtttcccgaagctgagaataggaagtggacaattatggcagtgatggtcagcctgctgactgattattcacctcagctccagaaaccaaagttctga

SEQ ID NO: 146 - DNA, 1065 bp, synthetic construct
  source 1..1065: mol_type = other DNA
atgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 147 - DNA, 1065 bp, synthetic construct
  source 1..1065: mol_type = other DNA
atgctgttcaacctgcgaatcctgctgaacaacgccgcttttcggaacgggcacaactttatggtgaggaactttcgctgcggacagcccctccagaataaggtccagctgaagggcagggacctgctgaccctgaaaaatttcacaggggaggaaatcaagtatatgctgtggctgtcagctgatctgaagttccggatcaagcagaagggcgaatatctgcctctgctccagggcaaaagcctggggatgatcttcgaaaagcgcagtactcggaccagactgtcaaccgagactggattcgctctgctgggaggacacccttgttttctgaccactcaggacattcacctgggagtgaacgagtccctgaccgacactgctcgcgtcctgagctctatggccgacgctgtgctagctcgagtctacaaacagtccgacctggataccctggccaaggaagcttctatcccaattattaacggcctgtcagacctgtatcaccccatccagattctggccgattacctgaccctccaggagcactattctagtctgaaagggctgacactgagttggattggggacggaaacaatatcctgcactctattatgatgtcagccgccaagtttggaatgcacctccaggctgcaaccccaaaaggctacgaacccgatgcctcagtgacaaagctggctgaacagtacgccaaagagaacggcactaagctgctgctgaccaacgaccctctggaggccgctcacggaggcaacgtgctgatcaccgatacctggattagtatgggacaggaggaagagaagaagaagcggctccaggccttccagggctaccaggtgacaatgaaaaccgctaaggtcgcagccagcgattggacctttctgcactgcctgcccagaaagcccgaagaggtggacgacgaggtcttctactctcccagaagcctggtgtttcccgaagctgagaataggaagtggacaattatggcagtgatggtcagcctgctgactgattattcacctcagctccagaaaccaaagttctga

SEQ ID NO: 148 - DNA, 1065 bp, synthetic construct
  source 1..1065: mol_type = other DNA
atgctgttcaacctgcgaatcctgctgaacaatgccgcttttcggaacgggcacaatttcatggtgaggaactttcgctgcggacagcccctccagaacaaggtccagctgaagggcagggacctgctgaccctgaaaaatttcacaggggaggaaatcaagtacatgctgtggctgtcagccgatctgaagttccggatcaagcagaagggcgaatatctgcctctgctccagggcaaaagcctggggatgatcttcgaaaagcgcagtactcggaccagactgtcaacagagactggattcgcactgctgggaggacacccatgttttctgaccacacaggacattcatctgggagtgaacgagtccctgaccgacacagcacgcgtcctgagctccatggctgatgcagtgctggctcgagtctacaaacagtctgacctggataccctggccaaggaagcttctatcccaatcattaatggcctgagtgacctgtatcaccccatccagattctggccgattacctgaccctccaggagcattattctagtctgaaagggctgacactgagctggattggggacggaaacaatatcctgcactccattatgatgagcgccgccaagtttggaatgcacctccaggctgcaaccccaaaaggctacgaacccgatgcctccgtgacaaagctggcagaacagtatgccaaagagaacggcactaagctgctgctgaccaatgaccctctggaggccgctcacggaggcaacgtgctgatcactgatacctggattagtatgggacaggaggaagagaagaagaagcggctccaggccttccagggctaccaggtgacaatgaaaactgctaaggtcgcagccagcgactggacctttctgcattgcctgcccagaaagcctgaagaggtggacgatgaggtcttctactcacccagaagcctggtgtttcctgaagctgagaataggaagtggacaatcatggcagtgatggtcagcctgctgactgattattcccctcagctccagaaaccaaagttctga

SEQ ID NO: 149 - DNA, 1065 bp, synthetic construct
  source 1..1065: mol_type = other DNA
atgcttttcaaccttcgcattctcctcaacaacgccgcgtttagaaacggacacaacttcatggtccgcaacttccgctgcggacagccgctgcagaacaaggtccagctcaagggtcgggatctcctgacgctgaagaactttaccggcgaagagattaagtacatgctgtggctgtccgccgaccttaagttccggatcaagcagaagggcgaataccttcccctgctgcaaggaaagtccctgggcatgatcttcgagaagcgcagtaccagaaccagactctccactgaaaccgggttcgcgctgcttggcggccacccgtgtttcctcactacgcaagacatccatcttggcgtgaacgagtcccttaccgacaccgccagggtgctgtcaagcatggccgacgccgtccttgcgcgcgtgtacaagcagtcagaccttgatactctggccaaggaagcctccatccctattatcaacggcctatccgacctttaccacccgatccagatcctcgctgactacctgaccctgcaagaacactacagcagcctcaagggactgactctgtcctggatcggcgacgggaacaacatcctgcactcaatcatgatgagcgcagccaagttcggcatgcatctccaagccgctacacccaagggttatgaaccggacgcctctgtgaccaagttggcagaacagtacgccaaggagaacggtactaagctccttttaaccaacgaccccctcgaagcagcccatggcgggaatgtgctcattaccgatacctggatttcgatgggccaggaggaggagaagaagaagcggctgcaggcgttccagggctaccaggtcaccatgaaaactgccaaagtggccgcctcggattggacctttctccactgcctgcctcggaagcctgaggaggtggacgacgaagtgttctactccccacggtccctcgtgttccccgaggccgaaaataggaagtggaccatcatggccgtgatggtgtccctcttgaccgattacagcccgcagcttcagaagcctaaattctag

SEQ ID NO: 150 - DNA, 1065 bp, synthetic construct
  source 1..1065: mol_type = other DNA
atgcttttcaatcttcgcatcctgttgaacaacgccgccttccgcaatggtcacaacttcatggtccggaacttcagatgtggacagcctctccaaaacaaggtccagctgaagggaagggacctcttaaccctcaaaaactttactggagaggagatcaagtacatgctgtggcttagcgccgaccttaagttccggatcaagcagaagggagagtacctcccgctgctgcaaggaaagagtcttggaatgatcttcgagaagcggtccaccagaactcgcctctccactgaaaccggattcgcactcctgggtggacacccgtgctttctgaccacccaagacatccacctcggagtgaacgagagcctcacggacaccgcgagagtgctgtcatccatggccgacgccgtgcttgcacgggtctacaagcagtccgatctggacactcttgccaaggaagcctccattcctatcattaacggtctgtcggatctgtaccacccgattcagatccttgcggactacctcacacttcaagaacactattcaagcctaaagggtctgaccctgtcctggatcggagatggaaacaacattctccattccatcatgatgagcgctgccaagttcggaatgcatctccaagcagcgactcctaagggttacgagccggacgcctcagtgactaagctggccgagcagtacgccaaggagaacggtaccaaactgttgcttactaacgacccgcttgaagcggcccatggaggaaacgtgctgattaccgacacctggatttcgatgggacaggaagaggagaagaagaagcggctccaggcgttccagggataccaggtcaccatgaaaacggccaaagtggccgctagcgattggacctttctgcactgcctcccgcgcaagcctgaagaagtggacgacgaagtgttctactcccctcgctctcttgtgttcccggaagccgaaaacaggaagtggaccatcatggccgtgatggtgtccctcctgaccgattacagcccgcagctgcagaagcctaagttctag

SEQ ID NO: 151 - DNA, 1065 bp, synthetic construct
  source 1..1065: mol_type = other DNA
atgcttttcaatctccgcatcctcctcaacaacgccgcgtttagaaacggccacaacttcatggtccggaacttcagatgtggccagccgcttcagaacaaggtccagctcaagggccgggatcttctgaccctgaagaactttactggcgaagaaatcaagtacatgctctggctctccgccgacttgaagttccgcattaagcagaagggggaataccttccgctgctgcaaggaaagtcgctcggcatgatctttgagaagcgctcaacccgcaccaggctgtccactgaaaccgggttcgcgctgcttggtggccacccctgcttcctgaccacccaagacattcacctcggagtgaacgaatcgctcactgatactgcccgggtgctgtcgtcgatggccgatgcagtgctggccagggtgtacaaacagtccgatctggacactctggccaaggaggcgtccatccctattatcaacggcctttccgacctctaccacccgattcagatccttgccgattacctcaccctgcaagaacactactcgtcactgaagggtctgaccttgtcctggatcggcgacggcaacaacatcctccattccattatgatgtccgccgccaaattcggcatgcatcttcaagccgcaacccctaagggttacgagccggacgcttccgtgaccaagctcgccgagcagtacgctaaggagaacggaaccaagcttctgctgactaacgaccccctagaggcagcccacgggggcaacgtgcttattactgacacctggatctccatgggacaggaagaagagaagaagaagcggttacaggcgttccagggctatcaggtcaccatgaaaaccgccaaggtcgctgcctcggactggaccttcctgcattgcctgcctcgcaagcccgaagaagtggacgacgaggtgttctactcgccacggtcccttgtgttccctgaggccgagaatagaaagtggaccattatggccgtgatggtgtcccttctcaccgactactcgccgcaactgcagaaacccaagttctag

SEQ ID NO: 152 - DNA, 1065 bp, synthetic construct
  source 1..1065: mol_type = other DNA
atgcttttcaatcttcgcatcctcctcaacaacgccgccttccggaacggtcacaacttcatggtccggaacttccgctgcggccagccgctccaaaacaaagtgcagcttaagggccgcgatctcctgaccctgaagaacttcaccggagaggaaatcaagtacatgctgtggctctcggcggacctgaagtttaggattaagcagaagggggagtatctgccgctgctccaagggaagtcccttggcatgatcttcgaaaagaggtccacccggactcggctcagcaccgaaacaggttttgcacttctggggggccacccgtgcttcctgacgacccaggacatccatctgggtgtcaacgagagtttgaccgacactgccagagtgctgtcatccatggcggacgcggtgctcgcgagagtgtacaagcagtccgatcttgacaccctggcaaaagaggcttcaatcccgatcattaacggactctcggatctgtaccaccctatccaaatcttggccgactacctgaccctgcaagaacactacagctccctgaagggcctgactctttcctggattggcgatggaaacaacattctccattctattatgatgtccgccgccaagttcggcatgcaccttcaagccgccaccccgaagggctacgaacctgacgcctccgtgactaagctagccgaacagtacgctaaggagaacggcactaagcttctccttaccaacgatccgctggaggcggcccatggcggaaatgtgcttatcaccgacacctggattagcatggggcaggaagaagagaagaagaaacggctccaggcattccagggctaccaggtcaccatgaaaactgccaaggtcgccgctagcgactggaccttcctccactgtctgcctcgcaagcctgaagaagtggacgacgaggtgttctactccccgcgctccctcgtgtttcctgaggccgagaacagaaagtggaccatcatggccgtgatggtgtcattacttacggactacagcccgcagctgcagaagccgaagttctag

SEQ ID NO: 153 - DNA, 1065 bp, synthetic construct
  source 1..1065: mol_type = other DNA
atgctttttaacttgagaatccttctgaacaacgccgctttccgcaacggtcataacttcatggtccggaacttcagatgtggccagcccctccaaaacaaagtgcagctgaagggccgggaccttcttacgctgaagaatttcaccggcgaagaaatcaagtacatgctctggctgtccgccgatcttaagttccgcattaagcagaagggggaatacctcccgctgctgcaagggaagtcgctgggcatgatttttgagaagcggtcaactcgcacccgcctgtccactgaaactggattcgcactgctcggtggccatccctgcttcctgaccacccaagacatccacctcggcgtgaacgagtccctgactgacaccgcccgggtcttatcctcgatggccgatgctgtgcttgcgagggtgtacaagcagtccgacctcgacacactcgcgaaggaggcctccatccccatcatcaacggcctgtccgacctttaccacccaattcagatcctcgccgattacctgaccctgcaagagcactactcgtcgctcaaggggcttaccctctcgtggattggcgacggcaacaacatccttcactccatcatgatgtcggcagcgaagttcggcatgcatctgcaagccgccacgcctaagggttatgaaccggatgcctcagtgaccaagctcgccgaacagtacgcgaaagagaatggaaccaagctacttctgaccaacgaccccctggaggccgctcacggcggcaacgtcctcattaccgatacttggatttcgatgggacaggaagaggaaaagaagaagagactgcaggcgttccagggataccaggtcaccatgaaaactgccaaagtggcagcctccgactggaccttccttcactgcctgccgaggaagcctgaagaggtggacgacgaggtgttctactccccgcgctccttggtgtttcctgaggccgaaaaccggaagtggactatcatggccgtgatggtgtccctcctcaccgactactcgccgcaactgcagaagcctaagttctag

SEQ ID NO: 154 - DNA, 1065 bp, synthetic construct
  source 1..1065: mol_type = other DNA
atgttattcaaccttagaattctccttaacaacgccgccttccggaatgggcataactttatggtccgcaatttccgctgtggacagcctctgcaaaacaaggtccagctcaagggccgggatctgctgactctcaagaacttcactggggaagaaatcaagtacatgctctggctgagcgccgacctcaagttccgcatcaagcagaagggagagtacctcccgctgctccaagggaagtccctgggcatgatcttcgagaagagatccacccgcaccagactttccactgagactggcttcgccttgctgggaggccacccatgcttcctgacgacccaggacattcaccttggcgtgaacgagtccctgactgacaccgcaagggtgttgtcctcgatggccgacgccgtgcttgcccgggtgtacaagcagagcgatcttgacaccctggctaaggaagcttccattcccatcatcaacggtctgagcgacctgtaccacccgattcagatcctggcggactacctaaccctgcaagagcactatagctccctgaagggcctcacactttcatggatcggcgacggcaacaacatcctgcactctattatgatgagcgctgccaaattcggcatgcacctccaagccgccacgcctaaaggctacgagcccgacgcctcggtgaccaagcttgcggagcagtacgcgaaggaaaacggcaccaagctgcttctcaccaacgatcctctggaagcggcccatggtggcaacgtgctcattaccgacacttggatctccatgggacaggaggaggaaaagaagaagcggctccaggcgtttcagggttaccaggtcaccatgaaaaccgccaaggtcgcagcctccgactggaccttccttcattgccttccgcgcaagcccgaagaagtggacgatgaagtgttttactcacctcggtcactcgtgttcccggaagcagagaacaggaaatggaccattatggccgtgatggtgtccctgctcaccgattacagtccgcaactgcagaagcccaagttctag

SEQ ID NO: 155 - DNA, 1065 bp, synthetic construct
  source 1..1065: mol_type = other DNA
atgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcaaatcctggccgattacctcaccctgcaagaacactacagctccctgaagggtctgacattgtcctggatcggcgacggcaacaacattctccattccatcatgatgtccgccgcaaaattcggcatgcatcttcaagccgccacgcctaagggttacgaacccgacgcttccgtgactaagctcgccgagcagtacgctaaggagaacggaaccaagctgctgctgactaacgacccgctagaagcagcccacgggggcaacgtgcttattactgacacctggatctccatgggccaggaggaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcacttctcaccgactacagcccgcagcttcagaagcccaagttctag

SEQ ID NO: 156 - DNA, 1065 bp, synthetic construct
  source 1..1065: mol_type = other DNA
atgcttttcaatctccgcatcctccttaacaacgccgcgtttagaaacggccacaacttcatggtccggaacttcagatgtggccagccgcttcaaaacaaggtccagcttaagggccgggatctcctcacccttaaaaacttcaccggcgaagagatcaagtacatgctctggctctccgcggaccttaagttccgcattaagcagaagggggaataccttccgctgcttcaaggaaagagcctcggcatgatctttgagaagcgctcaaccaggaccaggctttctactgaaactgggttcgcgcttctcggcggtcatccctgcttcctcacgacccaagacatccacctcggagtgaacgaatccctcacggatactgcccgcgtgctttcgagcatggcagacgccgtgctcgcccgggtgtacaaacagtccgatctcgacactctcgccaaggaggcgtcaattcctattatcaacggtcttagtgacctttaccacccgatccagatcctcgccgattacctcacactccaagaacactacagctcccttaagggtcttaccctctcctggatcggcgacggcaacaacattctccactccatcatgatgtccgccgcaaagttcggcatgcatcttcaagccgccaccccgaagggctacgagcctgatgcttccgtgactaagctcgccgagcagtacgctaaggagaacggaaccaagcttcttctcactaacgacccactcgaagcagcccatgggggcaacgtgcttatcactgacacctggatctccatgggccaggaagaagagaagaagaagcggctccaggcgttccagggatatcaggtcaccatgaaaaccgccaaggtcgctgcctccgactggacctttctccactgcctccctcgcaaacctgaagaagtggacgacgaggtgttctactcgccccggagcctcgtgttccccgaggccgagaatagaaagtggaccattatggccgtgatggtgtcactcctcaccgactacagcccgcagcttcagaagcccaagttctag

SEQ ID NO: 157 - DNA, 1065 bp, synthetic construct
  source 1..1065: mol_type = other DNA
atgcttttcaatctccgcatcctccttaacaacgccgcgtttagaaacggacataacttcatggtccggaacttcagatgtggacagccgcttcaaaacaaggtccagctgaagggtcgggatcttctgaccctgaagaactttaccggagaagagatcaagtacatgctctggctctccgcggacttgaagttccgcattaagcagaagggagaatacctcccgctgcttcaaggaaagagcctcggaatgatttttgagaagcgctcaaccaggacccgcctttctactgaaactggattcgcgctgctgggtggacacccctgcttcctgacgacccaggacatccacctcggagtgaacgaatccctcactgataccgcccgggtgttatcgagcatggcagatgccgtgctggccagggtgtacaaacagtccgatctggacactctggccaaggaggcgtcaattcctatcatcaacggacttagtgacctctaccatccgattcaaatcctggccgactacctcaccctgcaagaacactacagctccctgaagggtctgacattgtcctggatcggagatggaaacaacattctccactccatcatgatgtccgccgcaaaattcggaatgcatcttcaagccgccacgcctaagggttacgaacccgacgcttccgtgactaagctcgccgagcagtacgctaaggagaacggtaccaagcttctcctgaccaacgacccactagaagcagcccacggtggaaacgtgcttattactgacacttggatctccatgggacaggaggaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccgcggagcctcgtgttccccgaggccgagaatagaaagtggaccatcatggccgtgatggtgtcactgctcaccgactacagcccgcagcttcagaagcccaagttctag

SEQ ID NO: 158 - DNA, 1065 bp, synthetic construct
  source 1..1065: mol_type = other DNA
atgcttttcaacctccgcattctcctcaacaacgctgccttccggaatggacataacttcatggtccggaacttcagatgcggacagccgcttcagaacaaggtccagcttaaggggagagatctccttaccctcaaaaacttcactggcgaagaaatcaagtacatgctctggcttagtgcggatctcaagttccgcatcaagcagaagggagaatacctcccgctccttcaaggaaagagcctcggcatgatttttgagaagaggtccaccagaactcgcctttcaaccgagactgggttcgccctgcttggcggtcacccctgcttcctcactacccaagacatccacctcggcgtgaacgagagccttaccgacaccgcccgcgtgctctcctcaatggccgacgctgtgctcgcccgggtgtacaagcagtccgaccttgatactctcgccaaggaggcctccatcccaattatcaacgggctctctgatctctaccaccctatccaaatcctcgcggactacctcaccctccaagagcactatagctcgctcaagggcctcaccctttcctggattggcgacggcaacaacattcttcactcgatcatgatgtccgccgccaagttcggcatgcatctccaagccgcgacccccaagggctacgagcctgacgcatccgtgaccaagctcgccgagcagtacgcgaaggaaaatggcaccaagcttcttctcaccaacgacccccttgaggccgctcatggcggcaacgtgctcatcactgacacttggatcagcatgggccaggaggaggaaaagaagaagcgccttcaggcattccagggttaccaggtcaccatgaaaaccgccaaagtggccgcctccgactggacctttcttcactgtctcccgcggaagcctgaagaagtggatgacgaagtgttttactcccctcggtcactcgtgttcccggaagcagaaaacaggaagtggaccattatggcggtcatggtgtccctcctcaccgactacagcccgcagcttcagaaacccaagttctag

SEQ ID NO: 159 - DNA, 1065 bp, synthetic construct
  source 1..1065: mol_type = other DNA
atgcttttcaatctccgcatcctccttaacaacgcagcgtttagaaacggtcacaacttcatggtccggaacttccgctgtggacagccgcttcaaaacaaggtccagctgaagggtcgggaccttctgaccctgaagaactttactggagaagagatcaagtacatgctttggctgtccgcggacttgaagttccgcattaagcagaagggagaataccttccgctgctccaaggaaagagcctgggaatgatctttgagaagcgctcaaccaggacccgcctttctactgaaactggattcgcgctgctgggtggtcacccttgcttcctgacgacccaggacattcacctcggagtgaacgagtccctcactgataccgccagagtgttatcgagcatggcagatgccgtgctggctagggtgtacaaacagtccgatctggacaccctggccaaggaggcatcaattcctattatcaacggacttagtgacctctaccatccgattcaaatcctggccgattacctcaccctgcaagaacactacagctccctgaagggtctgacattgtcctggatcggagatggaaacaacattctccattccatcatgatgtccgcggccaagttcggaatgcatctccaagccgccacgccgaaaggatacgagccggacgcttccgtgactaagctcgccgagcagtacgctaaggagaacggaaccaagcttctgctgactaacgacccgctagaagccgcccacggtggaaacgtgcttattactgacacctggatctccatgggacaggaagaagagaaaaagaagcggctgcaggcgttccagggatatcaggtcaccatgaaaaccgccaaggtcgccgcctccgactggaccttccttcactgcctgcctcggaagcctgaagaagtggacgacgaggtgttctactcgccgcggagcctcgtgttccctgaggccgagaatagaaagtggaccatcatggccgtgatggtgtcactcctcaccgactacagcccgcagcttcagaagcctaagttctag

SEQ ID NO: 160 - DNA, 1065 bp, synthetic construct
  source 1..1065: mol_type = other DNA
atgcttttcaatctccgcattctcctcaacaacgcagcctttagaaacggccacaacttcatggtccggaacttcagatgtggccagccgcttcagaacaaggtccagctcaagggccgggacctcctcaccctcaaaaactttaccggcgaagagatcaagtacatgctctggctttcggccgaccttaagttccgcatcaagcagaagggggaataccttccgctgcttcaaggaaagtccctcggcatgatctttgaaaagcgctcgaccaggacccgcctttccactgaaaccgggttcgcgcttctcggtggccacccctgcttcctcaccacccaagacattcacctcggagtgaacgaatcccttaccgataccgcaagagtgctttcgtcgatggccgatgccgtgcttgcgcgggtgtacaagcagtcagatctcgacactctcgccaaggaggcgtccattcctattatcaacggcctttccgacctttaccacccgattcagatcctcgccgattacctcaccctgcaagagcactactcgtcactcaagggtcttaccctctcctggatcggcgacggaaacaacatcctccattcgatcatgatgtccgccgccaaattcggcatgcacctccaagccgcgaccccgaagggttacgagcccgacgcttccgtgaccaagctcgccgaacagtacgctaaggaaaacggcaccaagctcctcctcactaacgaccctctcgaagcagcccatgggggcaacgtgctcattactgacacttggatctcgatgggccaggaagaggagaaaaagaagcggcttcaggcgttccagggatatcaggtcaccatgaaaaccgccaaggtcgctgcctcggactggaccttccttcactgccttccgcgcaagcctgaagaggtggacgatgaggtgttctactccccacggtcccttgtgttccccgaggccgagaataggaagtggaccatcatggccgtgatggtgtcgctcctcactgactactccccgcaacttcagaagcctaagttctag

SEQ ID NO: 161 - DNA, 1065 bp, synthetic construct
  source 1..1065: mol_type = other DNA
atgctgtttaatctgagaatacttctaaacaacgccgccttccggaatggccataactttatggttcggaatttccgctgcggccagccgctgcagaacaaggtccagctgaagggaagagacttgctgaccctcaagaacttcaccggagaagaaatcaagtatatgctgtggctgtccgccgacctgaaattccgcatcaagcagaagggcgaatatctgccgctgttgcaagggaagtccctggggatgatcttcgagaagaggtccaccagaacacggctttcaaccgaaaccgggtttgcactgctgggtggacacccctgttttctgaccactcaagatatccacctgggcgtgaacgagtcccttaccgacactgctagggtgttgtccagcatggccgatgccgtcctggctcgcgtgtacaagcagtccgacctggataccctggcaaaggaagcgtccattcccattatcaacgggctgtccgacctgtaccatccgattcaaatcctggcggactacctgactctgcaagagcattacagcagcttgaaggggcttactctctcgtggatcggcgacgggaacaacatcctgcactccatcatgatgtccgccgccaagttcgggatgcatttgcaagctgcgaccccgaaaggttacgagcccgatgctagcgtaactaagcttgccgaacagtacgccaaagagaatggtacaaaactgcttctgactaacgacccgctggaagcagcccacggcgggaacgtgctgataaccgacacctggatttcaatggggcaggaggaagagaagaagaagcgactgcaggcgttccaaggctatcaggttaccatgaaaaccgccaaagtggcagccagcgattggactttcctgcactgtctgccgcggaagcccgaggaagttgatgacgaagtattctactcaccccggagcctcgtgttccccgaggccgaaaaccggaagtggactattatggccgtgatggtgtcgctgttgaccgactacagcccgcaactgcagaagccgaagttttag

SEQ ID NO: 162 - DNA, 1065 bp, synthetic construct
  source 1..1065: mol_type = other DNA
atgcttttcaacctgaggatccttttgaacaacgccgcctttcgcaacggccacaactttatggtccgcaatttccgctgcgggcagccgctgcagaacaaggtccagctgaagggccgggatctgctgaccctgaagaacttcaccggggaggaaatcaagtacatgctttggctctccgccgatctgaagttcagaatcaagcagaagggagagtacctcccgttgctgcaaggaaagtcactcggaatgattttcgaaaagagaagcactaggacccgcctctcaactgaaaccgggttcgcgctgctcgggggccatccgtgtttcctgactacccaagacatccacctgggagtgaacgagtcgctgaccgacaccgcacgcgtgctgtcatccatggcggacgcagtgcttgcccgggtgtacaagcagtcggacctggacactcttgccaaggaggcatcaatccccatcattaacggactgtccgatctctaccacccgattcagatcctggctgactacctaaccctgcaagagcactactcaagcctgaaggggctgaccctgtcgtggatcggggacggcaacaacattctgcactccatcatgatgtcggcggctaagttcgggatgcatttgcaagcggcaactccgaagggttatgaacccgacgcctccgtgaccaagctggccgaacagtacgccaaggaaaacggaaccaagttgctgctgactaatgatcccctggaggcggcccacggggggaacgtgctgataaccgatacctggatctccatggggcaggaagaagagaagaaaaagcggctgcaggcattccagggataccaggtcaccatgaaaaccgcaaaagtggcagccagcgactggactttcctccattgcctgccgcgaaagccggaggaggtcgatgacgaggtgttctactccccgcggtcgctggtgttcccggaggcggaaaaccggaagtggaccattatggccgtgatggtgtcactcctgactgactacagcccgcaactgcagaagccgaagttctag

SEQ ID NO: 163 - DNA, 1074 bp, synthetic construct
  source 1..1074: mol_type = other DNA
atgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaa

SEQ ID NO: 164 - DNA, 1073 bp, synthetic construct
  source 1..1073: mol_type = other DNA
atgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaataagtga

SEQ ID NO: 165 - DNA, 1073 bp, synthetic construct; identical to SEQ ID NO: 164
  source 1..1073: mol_type = other DNA

SEQ ID NO: 166 - DNA, 1074 bp, synthetic construct; identical to SEQ ID NO: 163
  source 1..1074: mol_type = other DNA

SEQ ID NO: 167 - DNA, 1073 bp, synthetic construct
  source 1..1073: mol_type = other DNA
atgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcatccgtgaccaagttggcggaacagtacgcgaaggagaacggaaccaagctcctgctgactaacgacccgctcgaggctgcgcatgggggtaacgtgctgattacggacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctagataagtga

SEQ ID NO: 168 - DNA, 1073 bp, synthetic construct; identical to SEQ ID NO: 164
  source 1..1073: mol_type = other DNA

SEQ ID NO: 169 - DNA, 1074 bp, synthetic construct; identical to SEQ ID NO: 163
  source 1..1074: mol_type = other DNA

SEQ ID NO: 170 - DNA, 1073 bp, synthetic construct; identical to SEQ ID NO: 164
  source 1..1073: mol_type = other DNA

SEQ ID NO: 171 - DNA, 1073 bp, synthetic construct; identical to SEQ ID NO: 167
  source 1..1073: mol_type = other DNA

SEQ ID NO: 172 - DNA, 1074 bp, synthetic construct; identical to SEQ ID NO: 163
  source 1..1074: mol_type = other DNA

SEQ ID NO: 173 - DNA, 1073 bp, synthetic construct; identical to SEQ ID NO: 164
  source 1..1073: mol_type = other DNA

SEQ ID NO: 174 - DNA, 1073 bp, synthetic construct; identical to SEQ ID NO: 167
  source 1..1073: mol_type = other DNA

SEQ ID NO: 175 - DNA, 1065 bp, synthetic construct
  source 1..1065: mol_type = other DNA
atgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 176 - DNA, 1065 bp, synthetic construct; identical to SEQ ID NO: 175
  source 1..1065: mol_type = other DNA

SEQ ID NO: 177 - DNA, 1065 bp, synthetic construct; identical to SEQ ID NO: 175
  source 1..1065: mol_type = other DNA

SEQ ID NO: 178 - DNA, 1074 bp, synthetic construct
  source 1..1074: mol_type = other DNA
atgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaataagtgaa

SEQ ID NO: 179 - DNA, 1077 bp, synthetic construct
  source 1..1077: mol_type = other DNA
atgggcgtcttcaacctgcggatcctgctgaacaacgccgccttccggaacggccacaacttcatggtccgcaacttcagatgcggccagcccctgcagaacaaggtgcagctgaagggccgggacctgctgaccctgaagaacttcaccggcgaagagatcaagtacatgctgtggctgagcgccgacctgaagttccggatcaagcagaagggcgagtacctgcccctgctgcaaggcaagagcctgggcatgatcttcgagaagcggagcacccggacccggctgagcaccgagacaggctttgccctgctgggaggccacccctgctttctgaccacccaggacatccacctgggcgtgaacgagagcctgaccgacaccgccagagtgctgagcagcatggccgacgccgtgctggcccgggtgtacaagcagagcgacctggacaccctggccaaagaggccagcatccccatcatcaacggcctgagcgacctgtaccaccccatccagatcctggccgactacctgaccctgcaggaacactacagctccctgaagggcctgaccctgagctggatcggcgacggcaacaacatcctgcacagcatcatgatgagcgccgccaagttcggcatgcatctgcaggccgccacccccaagggctacgagcctgatgccagcgtgaccaagctggccgagcagtacgccaaagagaacggcaccaagctgctgctgaccaacgaccccctggaagccgcccacggcggcaacgtgctgatcaccgacacctggatcagcatgggccaggaagaggaaaagaagaagcggctgcaggccttccagggctaccaggtcacaatgaagaccgccaaggtggccgccagcgactggaccttcctgcactgcctgccccggaagcccgaagaggtggacgacgaggtgttctacagcccccggtccctggtgttccccgaggccgagaaccggaagtggaccattatggccgtgatggtgtccctgctgaccgactactccccccagctgcagaagcccaagttctagataagtgaa

SEQ ID NO: 180 - DNA, 1077 bp, synthetic construct
  source 1..1077: mol_type = other DNA
atgggcgtcttcaacctgcggatcctgctgaacaacgccgccttccggaacggccacaacttcatggtccgcaacttcagatgcggccagcccctgcagaacagggtgcagctgaagggccgggacctgctgaccctgaagaacttcaccggcgaagagatcaggtacatgctgtggctgagcgccgacctgaagttccggatcaagcagaagggcgagtacctgcccctgctgcaaggcaagagcctgggcatgatcttcgagaagcggagcacccggacccggctgagcaccgagacaggctttgccctgctgggaggccacccctgctttctgaccacccaggacatccacctgggcgtgaacgagagcctgaccgacaccgccagagtgctgagcagcatggccgacgccgtgctggcccgggtgtacaagcagagcgacctggacaccctggccaaagaggccagcatccccatcatcaacggcctgagcgacctgtaccaccccatccagatcctggccgactacctgaccctgcaggaacactacagctccctgaagggcctgaccctgagctggatcggcgacggcaacaacatcctgcacagcatcatgatgagcgccgccaagttcggcatgcatctgcaggccgccacccccaagggctacgagcctgatgccagcgtgaccaagctggccgagcagtacgccaaagagaacggcaccaagctgctgctgaccaacgaccccctggaagccgcccacggcggcaacgtgctgatcaccgacacctggatcagcatgggccaggaagaggaaaagaagaagcggctgcaggccttccagggctaccaggtcacaatgaagaccgccaaggtggccgccagcgactggaccttcctgcactgcctgccccggaagcccgaagaggtggacgacgaggtgttctacagcccccggtccctggtgttccccgaggccgagaaccggaagtggaccattatggccgtgatggtgtccctgctgaccgactactccccccagctgcagaagcccaagttctagataagtgaa

SEQ ID NO: 181 - DNA, 1077 bp, synthetic construct
  source 1..1077: mol_type = other DNA
atgctggtcttcaacctgcggatcctgctgaacaacgccgccttccggaacggccacaacttcatggtccgcaacttcagatgcggccagcccctgcagaacagggtgcagctgaagggccgggacctgctgaccctgaagaacttcaccggcgaagagatcaggtacatgctgtggctgagcgccgacctgaagttccggatcaagcagaagggcgagtacctgcccctgctgcaaggcaagagcctgggcatgatcttcgagaagcggagcacccggacccggctgagcaccgagacaggctttgccctgctgggaggccacccctgctttctgaccacccaggacatccacctgggcgtgaacgagagcctgaccgacaccgccagagtgctgagcagcatggccgacgccgtgctggcccgggtgtacaagcagagcgacctggacaccctggccaaagaggccagcatccccatcatcaacggcctgagcgacctgtaccaccccatccagatcctggccgactacctgaccctgcaggaacactacagctccctgaagggcctgaccctgagctggatcggcgacggcaacaacatcctgcacagcatcatgatgagcgccgccaagttcggcatgcatctgcaggccgccacccccaagggctacgagcctgatgccagcgtgaccaagctggccgagcagtacgccaaagagaacggcaccaagctgctgctgaccaacgaccccctggaagccgcccacggcggcaacgtgctgatcaccgacacctggatcagcatgggccaggaagaggaaaagaagaagcggctgcaggccttccagggctaccaggtcacaatgaagaccgccaaggtggccgccagcgactggaccttcctgcactgcctgccccggaagcccgaagaggtggacgacgaggtgttctacagcccccggtccctggtgttccccgaggccgagaaccggaagtggaccattatggccgtgatggtgtccctgctgaccgactactccccccagctgcagaagcccaagttctagataagtgaa

SEQ ID NO: 182 - DNA, 1074 bp, synthetic construct
  source 1..1074: mol_type = other DNA
atgctgttcaacctgaggatcctgctgaacaacgcagctttcaggaacggccacaacttcatggtgaggaacttccggtgcggccagcccctgcagaacaaggtgcagctgaagggcagggacctgctgaccctgaagaacttcaccggagaggagatcaagtacatgctgtggctgagcgcagacctgaagttcaggatcaagcagaagggagagtacctgcccctgctgcaggggaagtccctgggcatgatcttcgagaagaggagtaccaggaccaggctgagcaccgaaaccggcttcgccctgctgggaggacacccctgcttcctgaccacccaggacatccacctgggcgtgaacgagagtctgaccgacaccgccagggtgctgtctagcatggccgacgccgtgctggccagggtgtacaagcagtcagacctggacaccctggctaaggaggccagcatccccatcatcaacggcctgagcgacctgtaccaccccatccagatcctggctgactacctgaccctgcaggagcactacagctctctgaagggcctgaccctgagctggatcggcgacgggaacaacatcctgcacagcatcatgatgagcgccgccaagttcggcatgcacctgcaggccgctacccccaagggttacgagcccgacgccagcgtgaccaagctggcagagcagtacgccaaggagaacggcaccaagctgctgctgaccaacgaccccctggaggccgcccacggaggcaacgtgctgatcaccgacacctggatcagcatgggacaggaggaggagaagaagaagcggctgcaggctttccagggttaccaggtgaccatgaagaccgccaaggtggctgccagcgactggaccttcctgcactgcctgcccaggaagcccgaggaggtggacgacgaggtgttctactctcccaggagcctggtgttccccgaggccgagaacaggaagtggaccatcatggctgtgatggtgtccctgctgaccgactacagcccccagctgcagaagcccaagttctgaataagtgaa

SEQ ID NO: 183 - DNA, 1074 bp, synthetic construct
  source 1..1074: mol_type = other DNA
atgctgttcaacctgaggatcctgctgaacaacgcagctttcaggaacggccacaacttcatggtgaggaacttccggtgcggccagcccctgcagaacaaggtgcagctgaagggcagggacctgctgaccctgaagaacttcaccggagaggagatcaagtacatgctgtggctgagcgcagacctgaagttcaggatcaagcagaagggagagtacctgcccctgctgcaggggaagtccctgggcatgatcttcgagaagaggagtaccaggaccaggctgagcaccgaaaccggcttcgccctgctgggaggacacccctgcttcctgacgacccaggacatccacctcggagtgaacgaatccctcaccgataccgcccgggtgttatcaagcatggcagatgccgtgctggccagggtgtacaaacagtccgatctggacactctggctaaggaggccagcatccccatcatcaacggcctgagcgacctgtaccaccccatccagatcctggctgactacctgaccctgcaggagcactacagctctctgaagggcctgaccctgagctggatcggcgacgggaacaacatcctgcactccatcatgatgtccgccgcgaagttcggaatgcatctgcaagccgccacgccaaaaggatacgaaccggatgcgcccgtgacaaagttggcggaacagtacgctaaggagaacggaaccaagctgctgctgaccaacgaccccctggaggccgcccacggaggcaacgtgctgatcaccgacacctggatcagcatgggacaggaggaggagaagaagaagcggctgcaggctttccagggttaccaggtgaccatgaagaccgccaaggtggctgccagcgactggaccttcctgcactgcctgcccaggaagcccgaggaggtggacgacgaggtgttctactctcccaggagcctggtgttccccgaggccgagaacaggaagtggaccatcatggctgtgatggtgtccctgctgaccgactacagcccccagctgcagaagcccaagttctgaataagtgaa

SEQ ID NO: 184 - DNA, 1074 bp, synthetic construct; identical to SEQ ID NO: 178
  source 1..1074: mol_type = other DNA

SEQ ID NO: 185 - DNA, 1074 bp, synthetic construct; identical to SEQ ID NO: 178
  source 1..1074: mol_type = other DNA

SEQ ID NO: 186 - DNA, 1065 bp, synthetic construct
  source 1..1065: mol_type = other DNA
atgcttttcaacttgagaatcctgctgaacaacgccgcctttcgcaacggtcacaattttatggtcagaaacttcagatgcggacagcccctccaaaacaaggtccagctgaagggccgcgatctcctcaccctgaagaacttcacgggggaggagatcaagtacatgctgtggctctccgctgacctgaagttcaggatcaagcagaagggagaatatctgccgctgctgcaagggaagtccctggggatgattttcgagaagcggagcacccggactcggctctccactgaaactggtttcgcccttctgggcggtcacccctgcttcctgaccactcaagacattcacctcggagtgaacgagtccttgactgacaccgcccgggtgctgtcgagcatggcagacgccgtgctagcccgcgtgtacaagcagtcagacctcgataccctggccaaggaggcttcgatcccgatcatcaacgggttgtccgacctgtaccacccgattcagattctcgccgactacctcaccctgcaagagcattacagctccctgaaggggcttaccctgtcctggattggcgacggaaacaacatcctgcactccattatgatgtcggcggccaagttcggcatgcacctccaagccgcgacccctaagggttacgaaccagacgcgtcagtgactaagctggccgaacagtacgcaaaggaaaatggcacgaagctgctcctgaccaacgatccgttggaagccgcccatggcggaaatgtgctcatcaccgacacctggatctcgatgggacaggaggaagagaagaagaagcggctgcaggcgttccagggctaccaggtcaccatgaaaactgccaaggtggccgccagcgactggaccttcctgcactgccttccgcgcaagcctgaggaggtggacgatgaagtgttctactctccacggtccctggtgttccccgaggcggagaaccgcaaatggaccatcatggctgtgatggtcagcctgctgaccgattacagccctcagttgcaaaagccgaagttttga

SEQ ID NO: 187 - DNA, 1065 bp, synthetic construct
  source 1..1065: mol_type = other DNA
atgctgttcaacctccgcatcctcctcaacaacgccgcattcagaaacgggcacaacttcatggtcagaaacttccgctgcgggcaacccctacaaaacaaggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 188 - DNA, 1074 bp, synthetic construct
  source 1..1074: mol_type = other DNA
atgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctgaataagtaga

SEQ ID NO: 189 - DNA, 1074 bp, synthetic construct
  source 1..1074: mol_type = other DNA
atgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcatccgtgaccaagttggcggaacagtacgcgaaggagaacggaaccaagctcctgctgactaacgacccgctcgaggctgcgcatgggggtaacgtgctgattacggacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctgaataagtaga

SEQ ID NO: 190 - DNA, 1074 bp, synthetic construct
  source 1..1074: mol_type = other DNA
atgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaataagtaga

SEQ ID NO: 191 - DNA, 1065 bp, synthetic construct; identical to SEQ ID NO: 136
  source 1..1065: mol_type = other DNA

SEQ ID NO: 192 - DNA, 1065 bp, synthetic construct; identical to SEQ ID NO: 136
  source 1..1065: mol_type = other DNA

SEQ ID NO: 193 - DNA, 1065 bp, synthetic construct; identical to SEQ ID NO: 136
  source 1..1065: mol_type = other DNA

SEQ ID NO: 194 - DNA, 1065 bp, synthetic construct; identical to SEQ ID NO: 136
  source 1..1065: mol_type = other DNA

SEQ ID NO: 195 - DNA, 1065 bp, synthetic construct; identical to SEQ ID NO: 136
  source 1..1065: mol_type = other DNA

SEQ ID NO: 196 - DNA, 1065 bp, synthetic construct
  source 1..1065: mol_type = other DNA
atggtgttcaacctccgcatcctcctcaacaacgccgcattcagaaacgggcacaacttcatggtcagaaacttccgctgcgggcaacccctacaaaacaaggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 197 - DNA, 1065 bp, synthetic construct
  source 1..1065: mol_type = other DNA
atggtgttcaacctccgcatcctcctcaacaacgccgcattcagaaacgggcacaacttcatggtcagaaacttccgctgcgggcaacccctacaaaaccgggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 198 - DNA, 1065 bp, synthetic construct
  source 1..1065: mol_type = other DNA
atggtgttcaacctccgcatcctcctcaacaacgccgcattcagaaacgggcacaacttcatggtcagaaacttccgctgcgggcaacccctacaaaaccgggtccagctcaaggggcgggacctcctgaccctgaagaacttcaccggcgaagagatccggtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 199 - DNA, 1065 bp, synthetic construct
  source 1..1065: mol_type = other DNA
atgctggtcaacctccgcatcctcctcaacaacgccgcattcagaaacgggcacaacttcatggtcagaaacttccgctgcgggcaacccctacaaaacaaggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 200 - DNA, 1065 bp, synthetic construct
  source 1..1065: mol_type = other DNA
atgctggtcaacctccgcatcctcctcaacaacgccgcattcagaaacgggcacaacttcatggtcagaaacttccgctgcgggcaacccctacaaaaccgggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 201 - DNA, 1065 bp, synthetic construct
  source 1..1065: mol_type = other DNA
atgctggtcaacctccgcatcctcctcaacaacgccgcattcagaaacgggcacaacttcatggtcagaaacttccgctgcgggcaacccctacaaaaccgggtccagctcaaggggcgggacctcctgaccctgaagaacttcaccggcgaagagatccggtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 202 - DNA, 1065 bp, synthetic construct
  source 1..1065: mol_type = other DNA
atgctggtcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 203 - DNA, 1065 bp, synthetic construct
  source 1..1065: mol_type = other DNA
atgctggtcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 204 - DNA, 1065 bp, synthetic construct
  source 1..1065: mol_type = other DNA
atgcttgtcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctag

SEQ ID NO: 205 - DNA, 1065 bp, synthetic construct
  source 1..1065: mol_type = other DNA
atgcttgtcaatctccgcatcctccttaacaacgccgcgtttagaaacggccacaacttcatggtccggaacttcagatgtggccagccgcttcaaaaccgggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctag

SEQ ID NO: 206 - DNA, 1068 bp, synthetic construct
  source 1..1068: mol_type = other DNA
atgggccttgtcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctag

SEQ ID NO: 207 - DNA, 1068 bp, synthetic construct
  source 1..1068: mol_type = other DNA
atgggccttgtcaatctccgcatcctccttaacaacgccgcgtttagaaacggccacaacttcatggtccggaacttcagatgtggccagccgcttcaaaaccgggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctag

SEQ ID NO: 208 - DNA, 1071 bp, synthetic construct
  source 1..1071: mol_type = other DNA
atgggcggacttgtcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctag

SEQ ID NO: 209 - DNA, 1077 bp, synthetic construct
  source 1..1077: mol_type = other DNA
atggcccttttcaatctccgcatcctccttaacaacgccgcgtttagaaacggccacaacttcatggtccggaacttcagatgtggccagccgcttcaaggcaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaa

SEQ ID NO: 210 - DNA, 1077 bp, synthetic construct
  source 1..1077: mol_type = other DNA
atggcccttttcaatctccgcatcctccttaacaacgccgcgtttagaaacggccacaacttcatggtccggaacttcagatgtggccagccgcttcaaggccgggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaa

SEQ ID NO: 211 - DNA, 1077 bp, synthetic construct
  source 1..1077: mol_type = other DNA
atggcccttttcaatctccgcatcctccttaacaacgccgcgtttagaaacggccacaacttcatggtccggaacttcagatgtggccagccgcttcaaggccgggtccagctgaagggccgggatcttctgaccctgaagaactttactggcgaagagatcaggtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaa

SEQ ID NO: 212 - DNA, 1077 bp, synthetic construct
  source 1..1077: mol_type = other DNA
atggcccttgtcaatctccgcatcctccttaacaacgccgcgtttagaaacggccacaacttcatggtccggaacttcagatgtggccagccgcttcaaggcagggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctaagtgaataga

SEQ ID NO: 213 - DNA, 1077 bp, synthetic construct
  source 1..1077: mol_type = other DNA
atggcccttttcaatctccgcatcctccttaacaacgccgcgtttagaaacggccacaacttcatggtccggaacttcagatgtggccagccgcttcaagtcaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaa

SEQ ID NO: 214 - DNA, 1077 bp, synthetic construct
  source 1..1077: mol_type = other DNA
atggcccttttcaatctccgcatcctccttaacaacgccgcgtttagaaacggccacaacttcatggtccggaacttcagatgtggccagccgcttcaagtcagggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaa

SEQ ID NO: 215 - DNA, 1065 bp, synthetic construct; identical to SEQ ID NO: 175
  source 1..1065: mol_type = other DNA

SEQ ID NO: 216 - DNA, 1068 bp, synthetic construct
  source 1..1068: mol_type = other DNA
atgggagtattcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 217 - DNA, 1068 bp, synthetic construct
  source 1..1068: mol_type = other DNA
atgggagtattcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 218 - DNA, 1068 bp, synthetic construct
  source 1..1068: mol_type = other DNA
atgctggtattcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 219 - DNA, 1068 bp, synthetic construct; identical to SEQ ID NO: 216
  source 1..1068: mol_type = other DNA

SEQ ID NO: 220 - DNA, 1068 bp, synthetic construct; identical to SEQ ID NO: 217
  source 1..1068: mol_type = other DNA

SEQ ID NO: 221 - DNA, 1068 bp, synthetic construct; identical to SEQ ID NO: 218
  source 1..1068: mol_type = other DNA

SEQ ID NO: 222 - DNA, 1073 bp, synthetic construct
  source 1..1073: mol_type = other DNA
atgttgttcaacttgaggatcttgttgaacaacgccgccttcaggaacggacacaacttcatggtaaggaacttcaggtgcggacagcccttgcagaacaaagtacagttgaaaggaagggacttgttgacattgaaaaacttcacaggagaagaaatcaaatacatgttgtggttgtcggccgacttgaaattcaggatcaaacagaaaggagaatacttgcccttgttgcagggaaaatcgttgggaatgatcttcgaaaaaaggtcgacaaggacaaggttgtcgacagaaacaggattcgccttgttgggaggacacccctgcttcttgacaacacaggacatccacttgggagtaaacgaatcgttgacagacacagccagggtattgtcgtcgatggccgacgccgtattggccagggtatacaaacagtcggacttggacacattggccaaagaagcctcgatccccatcatcaacggattgtcggacttgtaccaccccatccagatcttggccgactacttgacattgcaggaacactactcgtcgttgaaaggattgacattgtcgtggatcggagacggaaacaacatcttgcactcgatcatgatgtcggccgccaaattcggaatgcacttgcaggccgccacacccaaaggatacgaacccgacgcctcggtaacaaaattggccgaacagtacgccaaagaaaacggaacaaaattgttgttgacaaacgaccccttggaagccgcccacggaggaaacgtattgatcacagacacatggatctcgatgggacaggaagaagaaaaaaaaaaaaggttgcaggccttccagggataccaggtaacaatgaaaacagccaaagtagccgcctcggactggacattcttgcactgcttgcccaggaaacccgaagaagtagacgacgaagtattctactcgcccaggtcgttggtattccccgaagccgaaaacaggaaatggacaatcatggccgtaatggtatcgttgttgacagactactcgccccagttgcagaaacccaaattctgaatagtgaa

SEQ ID NO: 223 - DNA, 1065 bp, synthetic construct
  source 1..1065: mol_type = other DNA
atgctgttcaacctgcgcatcctgctgaacaacgccgccttccgcaacggccacaacttcatggtgcgcaacttccgctgcggccagcccctgcagggcaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 224 - DNA, 1065 bp, synthetic construct
  source 1..1065: mol_type = other DNA
atgctgttcaacctgcgcatcctgctgaacaacgccgccttccgcaacggccacaacttcatggtgcgcaacttccgctgcggccagcccctgcagggccgggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 225 - DNA, 1065 bp, synthetic construct
  source 1..1065: mol_type = other DNA
atgctgttcaacctgcgcatcctgctgaacaacgccgccttccgcaacggccacaacttcatggtgcgcaacttccgctgcggccagcccctgcagggccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 226 - DNA, 1065 bp, synthetic construct; identical to SEQ ID NO: 223
  source 1..1065: mol_type = other DNA

SEQ ID NO: 227 - DNA, 1065 bp, synthetic construct; identical to SEQ ID NO: 175
  source 1..1065: mol_type = other DNA

SEQ ID NO: 228 - DNA, 1068 bp, synthetic construct; identical to SEQ ID NO: 218
  source 1..1068: mol_type = other DNA

SEQ ID NO: 229 - DNA, 1068 bp, synthetic construct; identical to SEQ ID NO: 218
  source 1..1068: mol_type = other DNA

SEQ ID NO: 230 - RNA, 21 nt, synthetic construct
  source 1..21: mol_type = other RNA
aaccaatcgaaagaaaccaaa

SEQ ID NO: 231 - RNA, 21 nt, synthetic construct
  source 1..21: mol_type = other RNA
ctctaatcaccaggagtaaaa

SEQ ID NO: 232 - RNA, 21 nt, synthetic construct
  source 1..21: mol_type = other RNA
gagagagatcttaacaaaaaa

SEQ ID NO: 233 - RNA, 21 nt, synthetic construct
  source 1..21: mol_type = other RNA
tgtgtaacaacaacaacaaca

SEQ ID NO: 234 - RNA, 21 nt, synthetic construct
  source 1..21: mol_type = other RNA
ccgcagtaggaagagaaagcc

SEQ ID NO: 235 - RNA, 21 nt, synthetic construct
  source 1..21: mol_type = other RNA
aaaaaaaaaagaaatcataaa

SEQ ID NO: 236 - RNA, 21 nt, synthetic construct
  source 1..21: mol_type = other RNA
gagagaagaaagaagaagacg

SEQ ID NO: 237 - RNA, 21 nt, synthetic construct
  source 1..21: mol_type = other RNA
caattaaaaatacttaccaaa

SEQ ID NO: 238 - RNA, 21 nt, synthetic construct
  source 1..21: mol_type = other RNA
gcaaacagagtaagcgaaacg

SEQ ID NO: 239 - RNA, 21 nt, synthetic construct
  source 1..21: mol_type = other RNA
gcgaagaagacgaacgcaaag

SEQ ID NO: 240 - RNA, 21 nt, synthetic construct
  source 1..21: mol_type = other RNA
ttaggactgtattgactggcc

SEQ ID NO: 241 - RNA, 21 nt, synthetic construct
  source 1..21: mol_type = other RNA
atcatcggaattcggaaaaag

SEQ ID NO: 242 - RNA, 21 nt, synthetic construct
  source 1..21: mol_type = other RNA
aaaacaaaagttaaagcagac

SEQ ID NO: 243 - RNA, 21 nt, synthetic construct
  source 1..21: mol_type = other RNA
tttatctcaaataagaaggca

SEQ ID NO: 244 - RNA, 21 nt, synthetic construct
  source 1..21: mol_type = other RNA
ggtggggaggtgagatttctt

SEQ ID NO: 245 - RNA, 21 nt, synthetic construct
  source 1..21: mol_type = other RNA
tgattaggaaactacaaagcc

SEQ ID NO: 246 - RNA, 21 nt, synthetic construct
  source 1..21: mol_type = other RNA
catttttcaatttcataaaac

SEQ ID NO: 247 - RNA, 21 nt, synthetic construct
  source 1..21: mol_type = other RNA
ttacttttaagcccaacaaaa

SEQ ID NO: 248 - RNA, 21 nt, synthetic construct
  source 1..21: mol_type = other RNA
ggcgtgtgtgtgtgttgttga

SEQ ID NO: 249 - RNA, 21 nt, synthetic construct
  source 1..21: mol_type = other RNA
gtggtgaaggggaaggtttag

SEQ ID NO: 250 - RNA, 21 nt, synthetic construct
  source 1..21: mol_type = other RNA
ttgtttttttttggtttggtt

SEQ ID NO: 251 - RNA, 1231 nt, synthetic construct
  source 1..1231: mol_type = other RNA
aggattattacatcaaaacaaaaagccgccaccatgctggtattcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcagcatctag

SEQ ID NO: 252 - RNA, 1231 nt, synthetic construct; identical to SEQ ID NO: 251
  source 1..1231: mol_type = other RNA

SEQ ID NO: 253 - RNA, 1228 nt, synthetic construct
  source 1..1228: mol_type = other RNA
aggattattacatcaaaacaaaaagccgccaccatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcagcatctag

SEQ ID NO: 254 - RNA, 1065 nt, synthetic construct
  source 1..1065: mol_type = other RNA
atgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 255 - RNA, 1068 nt, synthetic construct
  source 1..1068: mol_type = other RNA
atgctggtattcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 256 - RNA, 1065 nt, synthetic construct; identical to SEQ ID NO: 254
  source 1..1065: mol_type = other RNA

SEQ ID NO: 257 - RNA, 1068 nt, synthetic construct; identical to SEQ ID NO: 255
  source 1..1068: mol_type = other RNA

SEQ ID NO: 258 - RNA, 1068 nt, synthetic construct; identical to SEQ ID NO: 255
  source 1..1068: mol_type = other RNA

(19) United States
(12) Patent Application Publication
(10) Pub. No.: US 2025/0136953 A1
(43) Pub. Date: May 1, 2025

(54) COMPOSITIONS AND METHODS FOR THE TREATMENT OF ORNITHINE TRANSCARBAMYLASE DEFICIENCY

(71) Applicant: Arcturus Therapeutics, Inc., San Diego, CA (US)

(72) Inventors: Padmanabh Chivukula, San Diego, CA (US); Priya Prakash Karmali, San Diego, CA (US); Kiyoshi Tachikawa, San Diego, CA (US); Suezanne E. Parker, Ramona, CA (US); Marciano Rodriguez Sablad, San Diego, CA (US); Pattraranee Limphong, San Diego, CA (US); Yanjie Bao, San Diego, CA (US); Jerel Boyd Lee Vega, San Diego, CA (US)

(21) Appl. No.: 18/909,701

(22) Filed: Oct. 8, 2024

Related U.S. Application Data
(63) Continuation of application No. 17/191,247, filed on Mar. 3, 2021.
(60) Provisional application No. 62/984,764, filed on Mar. 3, 2020.

(51) Int. Cl. C12N 9/10; A61K 9/1271; A61K 9/1272; A61K 31/221; A61K 39/00; A61K 48/00

(52) U.S. Cl. CPC C12N 9/1018; A61K 9/1272; A61K 31/221; A61K 48/00; A61K 48/0033; A61K 9/1271; A61K 2039/51; C12Y 201/03003

(57) ABSTRACT

The present disclosure describes compositions and methods for treating ornithine transcarbamylase (OTC) deficiency. The compositions include a lipid formulation and messenger RNA (mRNA) encoding an OTC enzyme. The lipid formulations can comprise an ionizable cationic lipid in a lipid nanoparticle encapsulating the mRNA.

### CROSS-REFERENCES TO RELATED APPLICATIONS

This application is a continuation application of U.S. application Ser. No. 17/191,247, filed Mar. 3, 2021, which claims priority to U.S. Provisional Application No. 62/984,764, filed Mar. 3, 2020, which is incorporated herein by reference in its entirety.

### REFERENCE TO A SEQUENCE LISTING

The instant application contains a Sequence Listing which has been submitted electronically in XML format and is hereby incorporated by reference in its entirety. Said XML copy, created on Oct. 4, 2024 is named “2024 Oct. 4 Sequence_Listing_ST26 049386-529C01US.xml” and is 480,778 bytes in size.

### BACKGROUND

### Field

The present disclosure relates to the use of mRNA as a therapeutic in the treatment of disease. More specifically, the present disclosure relates to lipid nanoparticle compositions and methods for treating the urea cycle disorder ornithine transcarbamylase (OTC) deficiency.

### Background

The breakdown of amino acids by mammals results in the production of waste ammonia (NH₃). The buildup of ammonia in the body is a toxic hazard, and mammals have developed the urea cycle, a metabolic process for converting ammonia into urea ((NH₂)₂CO), which can then be safely secreted. The processes of the urea cycle mainly occur in the liver. Urea produced by the liver is then released into the bloodstream where it travels to the kidneys and is ultimately excreted in urine. When a mammal cannot adequately clear nitrogen from the body, a state of hyperammonemia can occur, which presents detrimental effects upon the body and can even result in brain damage or death.

Ornithine transcarbamylase (OTC) is one of six enzymes in the urea cycle that play a role in the breakdown of proteins and removal of ammonia from the body. This metabolic process primarily occurs in hepatocytes with OTC being found in the mitochondria. OTC is specifically responsible for converting carbamoyl phosphate and ornithine into citrulline. Native OTC mRNA encodes a mitochondrial signaling peptide (MSP) that is necessary to redirect the nascent pre-protein from the cytosol into the mitochondria. OTC protein exists as a precursor in the cytosol with the MSP redirecting the pre-peptide into the mitochondria, where it undergoes cleavage of the MSP and delivery of the functional protein into the mitochondrial matrix.

Deficiency of the OTC enzyme results in excessive accumulation of nitrogen, in the form of ammonia (hyperammonemia), in the blood. Excess ammonia, which is a neurotoxin, travels to the central nervous system through the blood, resulting in the symptoms and physical findings associated with OTC deficiency. These symptoms can include vomiting, refusal to eat, progressive lethargy, and coma. If left untreated a hyperammonemic episode may progress to coma and life-threatening complications.

The severity and age of onset of OTC deficiency vary from person to person, even within the same family. A severe form of the disorder affects some infants, typically males, shortly after birth (neonatal period). A milder form of the disorder affects some children later in infancy. Both males and females may develop symptoms of OTC deficiency during childhood. Presently, the treatment of OTC deficiency is aimed at preventing excessive ammonia from being formed or from removing excessive ammonia during a hyperammonemic episode through the use of ammonia scavengers. Long-term therapy for OTC deficiency combines dietary restrictions and the stimulation of alternative methods of converting and excreting nitrogen from the body (alternative pathways therapy).

Dietary restrictions in individuals with OTC deficiency are aimed at limiting the amount of protein intake to avoid the development of excess ammonia. However, enough protein must be taken in by an affected infant to ensure proper growth. Infants with OTC deficiency are placed on a low protein, high calorie diet supplemented by essential amino acids.

In addition to dietary restrictions, individuals with OTC deficiency are treated by medications that stimulate the removal of nitrogen from the body. These medications provide an alternative method to the urea cycle in converting and removing nitrogen waste. These medications are unpalatable to many patients and are often administered via a tube that is placed in the stomach through the abdominal wall (gastrostomy tube) or a narrow tube that reaches the stomach via the nose (nasogastric tube).

In cases where there is no improvement or in cases where hyperammonemic coma develops, the removal of wastes by filtering an affected individual's blood through a machine (hemodialysis) may be necessary. Hemodialysis is also used to treat infants, children, and adults who are first diagnosed with OTC deficiency during hyperammonemic coma.

In some cases, liver transplantation may be an appropriate treatment option, which has been shown as a potential cure to the hyperammonemia in OTC deficiency. However, this operation is risky and may result in post-operative complications. Also, after liver transplantation, patients will need to follow a medication regimen throughout their lives for immunosuppression therapy.

In contrast to the current available treatments, the use of nucleic acids as therapeutic agents is an emerging field of medicine that presents both great challenges and great potential in the treatment of disease. Among the possible treatment avenues using nucleic acids, delivery of a messenger RNA (mRNA) encoding a desired enzyme has the potential to provide the necessary enzymatic activity in a targeted cell of a subject. However, mRNA-based therapies face several obstacles including achieving an adequate in vivo half-life of the mRNA, achieving an adequate translation efficiency of the mRNA such that an effective amount of enzyme is produced, minimizing adverse reactions to the mRNA (e.g., immunogenicity), and effectively delivering the mRNA to a target cell type.

One method for delivering nucleic acids to target cells that has been successfully employed is the encapsulation of the nucleic acid in a lipid formulation such as a liposome or a lipid nanoparticle. While the use of lipid formulations has had some success, it has been found that several of the lipids used in these formulations show low in vivo degradability and low potency.

In light of the above challenges, novel approaches and therapies are still needed for the treatment of OTC enzyme deficiency, and strategies are needed that overcome the challenges and limitations associated with, for example, mRNA-based therapies. Poor stability, effective translocation of the OTC to the mitochondria, and efficient delivery to the target cells remain significant challenges.

### SUMMARY

The present disclosure includes compositions and methods for the treatment of ornithine transcarbamylase (OTC) deficiency. The compositions of the present disclosure include a specially designed messenger RNA (mRNA) which shows enhanced in vivo stability and translation efficiency. In addition, the OTC protein translated from the specially designed mRNA shows enhanced uptake into hepatocytic mitochondria. The specially designed mRNA is further combined with a lipid formulation that includes a cationic lipid that offers a high potency (e.g., bioavailability) for delivery of the mRNA to hepatocytes as well as a high level of biodegradability, thus improving both the therapeutic effect of the composition and its safety profile.

In some embodiments, a composition is provided comprising an mRNA encoding an enzyme having ornithine transcarbamylase (OTC) activity; and a lipid formulation comprising an ionizable cationic lipid. In some embodiments, the ionizable cationic lipid is a compound of Formula (I) or any of its configurations described herein. In some embodiments, the ionizable cationic lipid is selected from

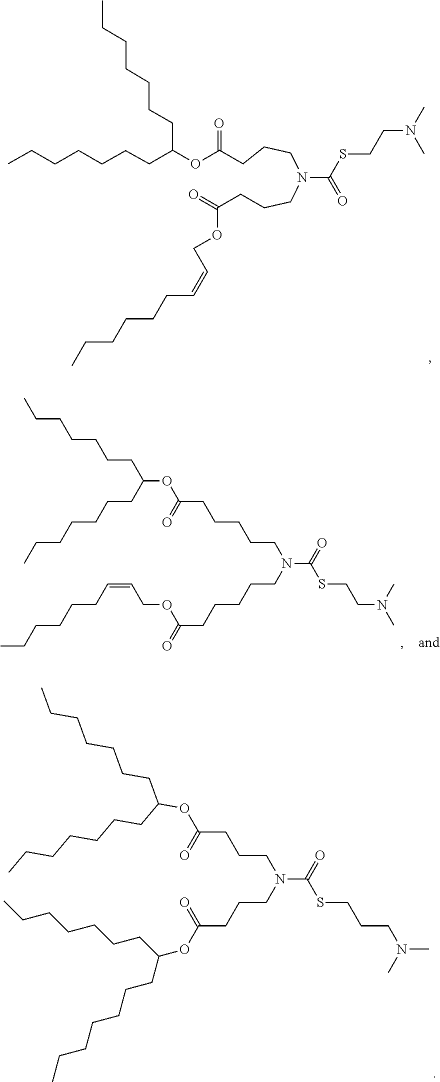

In some embodiments, the lipid formulation of the present disclosure further comprises a helper lipid (e.g., a neutral lipid or a noncationic lipid), a cholesterol, and/or a PEG-lipid.

In some embodiments, a method of treating OTC deficiency is provided comprising administering a composition described herein to a subject in need thereof.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

FIGS. 1A-1B show scatter plots illustrating ornithine transcarbamylase (OTC) protein expression in hepatocyte cell lines Hepa1,6 (mouse) and Hep3B (human) at 24 hours (FIG. 1A) and 48 hours (FIG. 1B) using In-Cell Western (ICW) assays.

FIG. 2 shows a scatter plot that illustrates the correlation of protein stability with compounds screened in Hepa1,6 cells at 24 hours in a first round of screening.

FIG. 3 shows a scatter plot that illustrates the correlation of protein stability with compounds screened in Hep3B cells at 24 hours in a first round of screening.

FIG. 4 shows a scatter plot illustrating the correlation of protein stability of compounds screened in human primary hepatocytes at 24 hours and 48 hours in a second round of screening (newly designed compounds based on the first round).

FIG. 5 shows a scatter plot illustrating the correlation of protein stability of compounds screened in human primary hepatocytes at 24 hours and 48 hours in a second round of screening (newly designed compounds based on the first round).

FIG. 6 shows a scatter plot illustrating the correlation of protein stability of compounds screened in human primary hepatocytes at 24 hours and 48 hours in a third round of screening (newly designed compounds based on rounds 1 and 2).

FIG. 7 shows a scatter plot illustrating the correlation of protein stability of compounds screened in human primary hepatocytes at 24 hours and 48 hours in a third round of screening (newly designed compounds based on rounds 1 and 2).

FIG. 8 is a plot illustrating OTC protein expression levels in human primary hepatocytes transfected with selected OTC mRNAs. Construct 1799.7 is an mRNA having the sequence of SEQ ID NO: 175 in which 100% of the uridines in SEQ ID NO: 175 are 5-methoxyuridine (5MeOU).

FIGS. 9A-9B show bar graphs depicting time course OTC expression levels in Spf/ash mice dosed with lipid-formulated human OTC (hOTC) mRNA at 10 mg/kg. Expression levels were measured by Multiple Reaction Monitoring (MRM) using heavy peptides specific for hOTC (FIG. 9A) or endogenous mouse OTC (FIG. 9B).

FIG. 10 is a bar graph depicting OTC expression levels in Spf/ash mice dosed at 3 mg/kg with OTC-mRNAs using two different uridine chemistries wherein 100% of the uridines are N¹-methylpseudouridine (N1MPU) and 100% of the uridines are 5-methoxyuridine (5MeOU).

FIG. 11 is a graph depicting OTC expression levels in Balb/c mice dosed with OTC mRNAs at three different doses and using two different uridine chemistries (N1MPU and 5MeOU).

FIG. 12 is a graph depicting urinary orotate levels measured in Spf/ash mice dosed with OTC mRNA 1799.7 at three different doses: 0.3 mg/kg, 1 mg/kg and 3 mg/kg. Construct 1799.7 is an mRNA having the sequence of SEQ ID NO: 175 wherein 100% of the uridines in SEQ ID NO: 175 are 5MeOU.

FIG. 13 is a scatter plot comparing human OTC expression levels and urinary orotate at 96 hours in Spf/ash mice dosed with OTC mRNAs at 1 mg/kg and 3 mg/kg using two different uridine chemistries (N1MPU and 5MeOU).

FIG. 14 is a western blot illustrating the protein expression levels of OTC mRNAs in Spf/ash mice dosed at 1 mg/kg and 3 mg/kg with selected OTC mRNAs.

FIG. 15 shows results of a western blot illustrating the protein expression levels in mitochondrial vs. cytosolic fractions of Spf/ash mice treated with selected OTC mRNAs.

FIG. 16 is a plot illustrating the time course of expression of urinary orotate levels in Spf/ash mice treated with selected OTC mRNA.

FIG. 17 is a plot illustrating the survival of OTC-deficient mice (Spf/ash) on a high protein diet during treatment with three different doses of OTC mRNA 1799.7.

FIG. 18 is a plot illustrating hOTC expression levels in male Balb/c mice dosed with OTC mRNAs (Construct 2262) having different modifications.

FIG. 19 is a graph that shows the levels of human OTC protein expression in wild-type mice for several different OTC mRNA lipid formulations at doses of 0.3 mg/kg, 1.0 mg/kg, and 3.0 mg/kg. The dotted line labeled as mOTC represents the baseline level of expression for mouse OTC in wildtype mice.

FIG. 20 is a graph that shows human OTC (hOTC) expression in tissue samples of wild-type mice for four different OTC mRNA lipid formulations at doses of 1.0 mg/kg and 3.0 mg/kg.

FIG. 21 is a graph that shows the survival of Spf/ash (OTC hypomorph) mice, after inducing hyperammonemia and being fed on a high protein diet (HPD), for a lipid formulation comprising Lipid #3 as described herein.

FIG. 22 is a graph that shows the survival of Spf/ash (OTC hypomorph) mice, after inducing hyperammonemia and being fed on a high protein diet (HPD), for a lipid formulation comprising Lipid #2 as described herein.

FIG. 23 is a graph that shows the survival of Spf/ash (OTC hypomorph) mice, after inducing hyperammonemia and being fed on a high protein diet (HPD), for a lipid formulation comprising Lipid #7 as described herein.

FIG. 24 is a graph that shows the survival rate of wild-type mice in a lipid tolerability study using OTC mRNA formulations having different ionizable cationic lipids described herein.

FIG. 25 is a graph that shows lipid clearance over time for tissue samples from mice dosed with an OTC mRNA lipid formulation comprising Lipid #7 described herein.

FIG. 26 is a graph that shows lipid clearance over time for tissue samples from mice dosed with an OTC mRNA lipid formulation comprising Lipid #3 described herein.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details.

In some embodiments, a composition is provided comprising an mRNA encoding an enzyme having ornithine transcarbamylase (OTC) activity; and a lipid formulation comprising an ionizable cationic lipid selected from

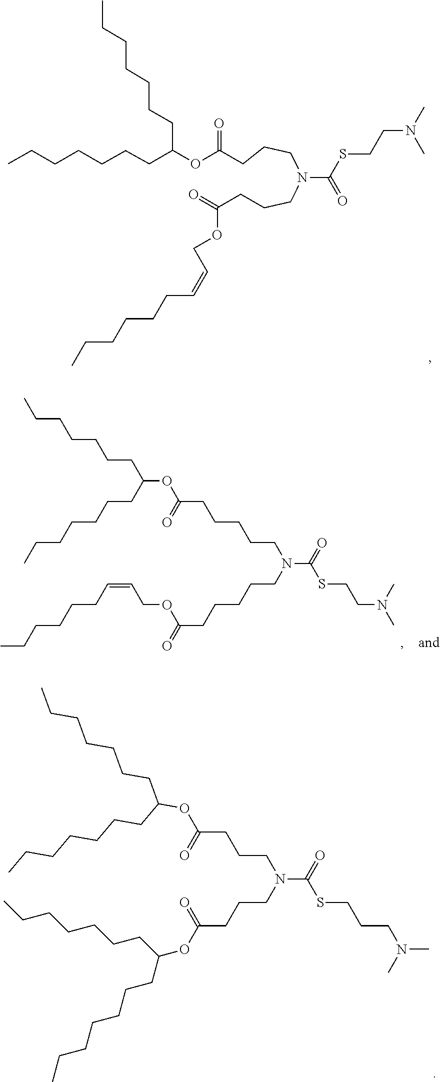

In some embodiments, the mRNA encodes an OTC enzyme having at least 95% identity to a sequence of SEQ ID NO: 3 or SEQ ID NO: 4. In some embodiments, the mRNA encodes an OTC enzyme consisting of a sequence of SEQ ID NO: 3. In some embodiments, the mRNA encodes an OTC enzyme consisting of a sequence of SEQ ID NO: 4. In some embodiments, the mRNA comprises a coding region having a sequence selected from the group consisting of SEQ ID NOs: 254-258. In some embodiments, the mRNA comprises a coding region having a sequence of SEQ ID NO: 254. In some embodiments, the mRNA comprises a coding region having a sequence of SEQ ID NO: 255. In some embodiments, the mRNA comprises a coding region having a sequence of SEQ ID NO: 256. In some embodiments, the mRNA comprises a coding region having a sequence of SEQ ID NO: 257. In some embodiments, the mRNA comprises a coding region having a sequence of SEQ ID NO: 258.

In some embodiments, the mRNA further comprises a 5′ untranslated region (5′ UTR) comprising a sequence of SEQ ID NO: 6.

In some embodiments, the mRNA further comprises a Kozak sequence having a sequence of SEQ ID NO: 23 or SEQ ID NO: 24.

In some embodiments, the mRNA further comprises a 3′ untranslated region (3′ UTR) comprising a sequence selected from SEQ ID NOs: 16-22.

In some embodiments, the mRNA further comprises a 3′ poly-adenosine (poly-A) tail comprising about 60 to about 125 consecutive adenine nucleotides.

In some embodiments, the mRNA further comprises a 5′ cap. In some embodiments, the 5′ cap is m⁷GpppGm having the structure of Formula Cap (IV) disclosed herein, wherein R¹ and R² are each OH, R³ is OCH₃, each L is a phosphate linked by phosphodiester bonds, mRNA is the mRNA encoding an enzyme having OTC activity linked at its 5′ end, and n is 1.

In some embodiments, the 5′ cap is m⁷GpppAmpG having the structure of Formula Cap (XI) disclosed herein, wherein R¹, R², and R⁴ are each OH, n is 1, each L is a phosphate linked by phosphodiester bonds, and mRNA is the mRNA encoding an enzyme having OTC activity linked at its 5′ end.

In some embodiments, the mRNA comprises a sequence selected from SEQ ID NOs: 1, 73, 119, and 251-253. In some embodiments, the mRNA comprises the sequence of SEQ ID NO: 1. In some embodiments, the mRNA comprises the sequence of SEQ ID NO: 73. In some embodiments, the mRNA comprises the sequence of SEQ ID NO: 119. In some embodiments, the mRNA comprises the sequence of SEQ ID NO: 251. In some embodiments, the sequence of SEQ ID NO: 252. In some embodiments, the mRNA comprises the sequence of SEQ ID NO: 253.

In embodiments, any one or more of the sequences described herein may be expressly excluded.

In some embodiments, about 1 to about 100% of the uridine nucleotides of the mRNA are 5-methoxy uridine or N¹-methyl pseudouridine. In some embodiments, 100% of the uridine nucleotides of the mRNA are 5-methoxy uridine. In some embodiments, 100% of the uridine nucleotides of the mRNA are N¹-methyl pseudouridine.

In some embodiments, the ionizable cationic lipid is

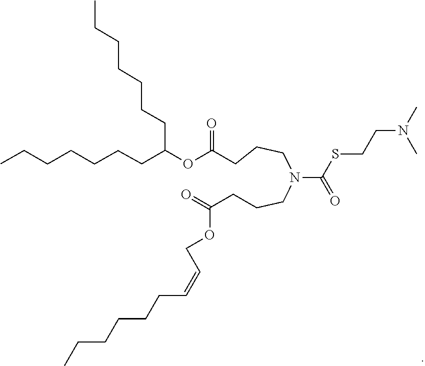 [SMILES: CCCCCC/C=C\COC(=O)CCCN(CCCC(=O)OC(CCCCCCC)CCCCCCC)C(=O)SCCN(C)C]

In some embodiments, the ionizable cationic lipid is

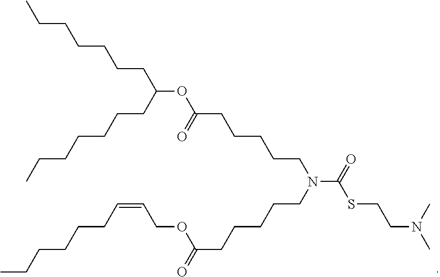 [SMILES: CCCCCC/C=C\COC(=O)CCCCCN(CCCCCC(=O)OC(CCCCCCC)CCCCCCC)C(=O)SCCN(C)C]

In some embodiments, the ionizable cationic lipid is

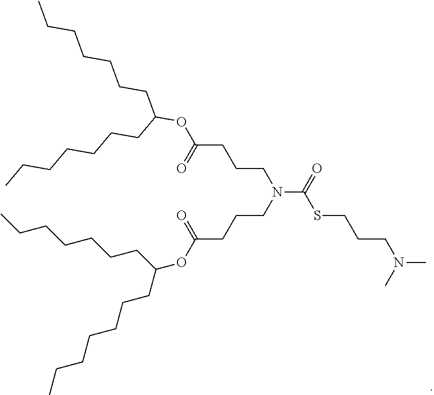 [SMILES: CCCCCCCC(CCCCCCC)OC(=O)CCCN(CCCC(=O)OC(CCCCCCC)CCCCCCC)C(=O)SCCCN(C)C]

In some embodiments, the lipid formulation comprises lipid nanoparticles. In some embodiments, the lipid nanoparticles have an average particle size of less than about 100 nm. In some embodiments, the lipid nanoparticles have an average particles size of about 55 nm to about 85 nm. In some embodiments, the lipid nanoparticles encapsulate at least about 50% of the mRNA. In some embodiments, the lipid nanoparticles encapsulate at least about 85% of the mRNA.

In some embodiments, the lipid formulation further comprises a helper lipid selected from dioleoylphosphatidyl ethanolamine (DOPE), dimyristoylphosphatidyl choline (DMPC), distearoylphosphatidyl choline (DSPC), dimyristoylphosphatidyl glycerol (DMPG), dipalmitoyl phosphatidylcholine (DPPC), and phosphatidylcholine (PC). In some embodiments, the helper lipid is distearoylphosphatidylcholine (DSPC).

In some embodiments, the lipid formulation further comprises cholesterol.

In some embodiments, the lipid formulation further comprises a polyethylene glycol (PEG)-lipid conjugate. In some embodiments, the PEG-lipid conjugate is PEG-DMG. In some embodiments, the PEG-DMG is PEG2000-DMG.

In embodiments, any one or more of the recited lipids may be expressly excluded.

In some embodiments, the lipid portion of the lipid formulation comprises about 48 mol % to about 66 mol % of the ionizable cationic lipid, about 2 mol % to about 12 mol % DSPC, about 25 mol % to about 42 mol % cholesterol, and about 0.5 mol % to about 3 mol % PEG2000-DMG. In some embodiments, the lipid portion of the lipid formulation comprises about 55 mol % to about 61 mol % of the ionizable cationic lipid, about 5 mol % to about 9 mol % DSPC, about 29 mol % to about 38 mol % cholesterol, and about 1 mol % to about 2 mol % PEG2000-DMG. In some embodiments, the lipid portion of the lipid formulation comprises about 56 mol % to about 58 mol % of the ionizable cationic lipid, about 6 mol % to about 8 mol % DSPC, about 31 mol % to about 34 mol % cholesterol, and about 1.25 mol % to about 1.75 mol % PEG2000-DMG. The concentration may be any value or subrange within the recited ranges, including endpoints. The ratio may be any value or subrange within the recited ranges, including endpoints.

In some embodiments, the composition has a total lipid: mRNA weight ratio of about 50:1 to about 10:1. In some embodiments, the composition has a total lipid: mRNA weight ratio of about 40:1 to about 20:1. In some embodiments, the composition has a total lipid: mRNA weight ratio of about 35:1 to about 25:1. In some embodiments, the composition has a total lipid: mRNA weight ratio of about 28:1 to about 32:1. In some embodiments, the composition has a total lipid: mRNA weight ratio of about 29:1 to about 31:1.

In some embodiments, the composition comprises a HEPES buffer at a pH of about 7.4. In some embodiments, the HEPES buffer is at a concentration of about 7 mg/mL to about 15 mg/mL. In some embodiments, the composition further comprises about 2.0 mg/mL to about 4.0 mg/mL of NaCl. In some embodiments, the composition further comprises one or more cryoprotectants. In some embodiments, the one or more cryoprotectants are selected from sucrose, glycerol, or a combination of sucrose and glycerol. In some embodiments, the composition comprises a combination of sucrose at a concentration of about 70 mg/mL to about 110 mg/mL and glycerol at a concentration of about 50 mg/mL to about 70 mg/mL.

In some embodiments, a method of producing an ornithine transcarbamylase (OTC) enzyme in a cell is provided comprising contacting the cell with any of the compositions described herein. In some embodiments, the cell is a hepatocyte.

In some embodiments, a method of treating ornithine transcarbamylase (OTC) deficiency is provided comprising administering a therapeutically effective amount of any of the compositions described herein to a subject in need thereof. In some embodiments, the subject is an adult. In some embodiments, the subject is a child. In some embodiments, an enzyme having OTC activity is produced in hepatocytes of the subject. In some embodiments, the administering comprises intravenous administration. In some embodiments, the composition is administered to the subject at least once per month. In some embodiments, the composition is administered to the subject at least twice per month. In some embodiments, the composition is administered to the subject in a dose of from about 0.2 mg of the mRNA per kg of the subject to about 10 mg of the mRNA per kg of the subject. The amount may be any value or subrange within the recited ranges, including endpoints.

In some embodiments, a method of expressing an ornithine transcarbamylase (OTC) enzyme in a mammal is provided comprising administering any of the compositions of the present disclosure to the mammal.

In some embodiments, the present disclosure provides for the use of any of the compositions described herein in the treatment of ornithine transcarbamylase (OTC) deficiency.

### Polynucleotides

The compositions and methods of the present disclosure include an mRNA that encodes an enzyme having ornithine transcarbamylase (OTC) activity. The mRNA can include several features that enhance its in vivo half-life and translation efficiency. In addition, the present disclosure provides for DNA scaffolds for producing an mRNA encoding an enzyme having OTC activity via transcription. The DNA scaffold can be any suitable form of DNA including a plasmid DNA. The polynucleotides contemplated by the present disclosure are further described in detail below.

In some embodiments, the OTC proteins encoded by the mRNA described herein are wildtype human OTC (hOTC). Preferably, the OTC proteins encoded by the mRNA described herein are produced from a heterologous mRNA construct comprising an open reading frame (ORF) also referred to herein as a “coding sequence” (CDS) encoding for an OTC protein. Preferably, the coding sequence is codon-optimized.

Preferably, a human OTC protein encoded by an mRNA described herein comprises a modified human OTC protein of SEQ ID NO: 4 shown in Table 1. SEQ ID NO: 4 has been modified from wild-type OTC of SEQ ID NO: 3 (Table 1) to remove one or more predicted ubiquitination sites resulting in a protein that is less susceptible to ubiquitination and degradation by ubiquitin ligases. The removal of predicted ubiquitination sites preferably comprises replacing N-terminus residues that have been found to support ubiquitination such as asparagine, arginine, leucine, lysine or phenylalanine with N-terminus residues that have been found to be stabilizing against ubiquitination such as alanine, glycine, methionine, serine, threonine, valine and proline. Stabilization of the modified OTC protein of SEQ ID NO: 4 in this manner is particularly advantageous for preserving the stability of the modified OTC protein during its transport from the cytosol to the mitochondria wherein it exerts its enzymatic activity.

Preferably, an OTC protein encoded by an mRNA described herein comprises a protein sequence that is at least about 70%, about 75%, about 80%, about 85%, about 90%, about 95% about 96%, about 97%, about 98%, about 99%, or about 100% identical to human wild type OTC protein of SEQ ID NO: 3 as shown in Table 1, while retaining the OTC protein activity of catalyzing the synthesis of citrulline (in the liver and small intestine) from carbamoyl phosphate and ornithine.

TABLE 1
Selected OTC Nucleotide and Peptide Sequences

| | |
|---|---|
| mRNA coding | AUGCUGUUUAAUCUGAGGAUCCUGUUAAACAAUGCAGCUUUUAGAAA |
| sequence for wild type | UGGUCACAACUUCAUGGUUCGAAAUUUUCGGUGUGGACAACCACUAC |
| human OTC (SEQ ID | AAAAUAAAGUGCAGCUGAAGGGCCGUGACCUUCUCACUCUAAAAAAC |
| NO: 1) | UUUACCGGAGAAGAAAUUAAAUAUAUGCUAUGGCUAUCAGCAGAUCU |
| | GAAAUUUAGGAUAAAACAGAAAGGAGAGUAUUUGCCUUUAUUGCAAG |
| | GGAAGUCCUUAGGCAUGAUUUUUGAGAAAAGAAGUACUCGAACAAGA |
| | UUGUCUACAGAAACAGGCUUUGCACUUCUGGGAGGACAUCCUUGUUU |
| | UCUUACCACACAAGAUAUUCAUUUGGGUGUGAAUGAAAGUCUCACGG |
| | ACACGGCCCGUGUAUUGUCUAGCAUGGCAGAUGCAGUAUUGGCUCGA |
| | GUGUAUAAACAAUCAGAUUUGGACACCCUGGCUAAAGAAGCAUCCAU |
| | CCCAAUUAUCAAUGGGCUGUCAGAUUUGUACCAUCCUAUCCAGAUCCU |
| | GGCUGAUUACCUCACGCUCCAGGAACACUAUAGCUCUCUGAAAGGUCU |
| | UACCCUCAGCUGGAUCGGGGAUGGGAACAAUAUCCUGCACUCCAUCAU |
| | GAUGAGCGCAGCGAAAUUCGGAAUGCACCUUCAGGCAGCUACUCCAAA |
| | GGGUUAUGAGCCGGAUGCUAGUGUAACCAAGUUGGCAGAGCAGUAUG |
| | CCAAAGAGAAUGGUACCAAGCUGUUGCUGACAAAUGAUCCAUUGGAA |
| | GCAGCGCAUGGAGGCAAUGUAUUAAUUACAGACACUUGGAUAAGCAU |
| | GGGACAAGAAGAGGAGAAGAAAAAGCGGCUCCAGGCUUUCCAAGGUU |
| | ACCAGGUUACAAUGAAGACUGCUAAAGUUGCUGCCUCUGACUGGACA |
| | UUUUUACACUGCUUGCCCAGAAAGCCAGAAGAAGUGGAUGAUGAAGU |
| | CUUUUAUUCUCCUCGAUCACUAGUGUUCCCAGAGGCAGAAAACAGAA |
| | AGUGGACAAUCAUGGCUGUCAUGGUGUCCCUGCUGACAGAUUACUCA |
| | CCUCAGCUCCAGAAGCCUAAAUUUUGA |
| DNA coding sequence | ATGCTGTTTAATCTGAGGATCCTGTTAAACAATGCAGCTTTTAGAAAT |
| for wild type human | GG |
| OTC (SEQ ID NO: 2) | TCACAACTTCATGGTTCGAAATTTTCGGTGTGGACAACCACTACAAAA |
| | TA |
| | AAGTGCAGCTGAAGGGCCGTGACCTTCTCACTCTAAAAAACTTTACCGG |
| | AGAAGAAATTAAATATATGCTATGGCTATCAGCAGATCTGAAATTTAGG |
| | ATAAAACAGAAAGGAGAGTATTTGCCTTTATTGCAAGGGAAGTCCTTAG |
| | GCATGATTTTTGAGAAAAGAAGTACTCGAACAAGATTGTCTACAGAAAC |
| | AGGCTTTGCACTTCTGGGAGGACATCCTTGTTTTCTTACCACACAAGATA |
| | TTCATTTGGGTGTGAATGAAAGTCTCACGGACACGGCCCGTGTATTGTCT |
| | AGCATGGCAGATGCAGTATTGGCTCGAGTGTATAAACAATCAGATTTGG |
| | ACACCCTGGCTAAAGAAGCATCCATCCCAATTATCAATGGGCTGTCAGA |
| | TTTGTACCATCCTATCCAGATCCTGGCTGATTACCTCACGCTCCAGGAAC |
| | ACTATAGCTCTCTGAAAGGTCTTACCCTCAGCTGGATCGGGGATGGGAA |
| | CAATATCCTGCACTCCATCATGATGAGCGCAGCGAAATTCGGAATGCAC |
| | CTTCAGGCAGCTACTCCAAAGGGTTATGAGCCGGATGCTAGTGTAACCA |
| | AGTTGGCAGAGCAGTATGCCAAAGAGAATGGTACCAAGCTGTTGCTGAC |
| | AAATGATCCATTGGAAGCAGCGCATGGAGGCAATGTATTAATTACAGAC |
| | ACTTGGATAAGCATGGGACAAGAAGAGGAGAAGAAAAAGCGGCTCCAG |
| | GCTTTCCAAGGTTACCAGGTTACAATGAAGACTGCTAAAGTTGCTGCCTC |
| | TGACTGGACATTTTTACACTGCTTGCCCAGAAAGCCAGAAGAAGTGGAT |
| | GATGAAGTCTTTTATTCTCCTCGATCACTAGTGTTCCCAGAGGCAGAAAA |
| | CAGAAAGTGGACAATCATGGCTGTCATGGTGTCCCTGCTGACAGATTAC |
| | TCACCTCAGCTCCAGAAGCCTAAATTTTGA |
| Human wild type OTC | MLFNLRILLNNAAFRNGHNFMVRNFRCGQPLQNKVQLKGRDLLTLKNFT |
| amino acid sequence | G |
| (The signal peptide for | EEIKYMLWLSADLKFRIKQKGEYLPLLQGKSLGMIFEKRSTRTRLSTETGFA |
| mitochondrial import is | LLGGHPCFLTTQDIHLGVNESLTDTARVLSSMADAVLARVYKQSDLDTLAK |
| underlined*) (SEQ ID | EASIPIFGLSDLYHPIQILADYLTLQEHYSSLKGLTLSWIGDGNNILHSIMMS |
| NO: 3) | AAKFGMHLQAATPKGYEPDASVTKLAEQYAKENGTKLLLTNDPLEAAHGG |
| | NVLITDTWISMGQEEEKKKRLQAFQGYQVTMKTAKVAASDWTFLHCLPRK |
| | PEEVDDEVFYSPRSLVFPEAENRKWTIMAVMVSLLTDYSPQLQKPKF |
| Modified OTC amino | MLVFNLRILLNNAAFRNGHNFMVRNFRCGQPLQNRVQLKGRDLLTLKNF |
| acid sequence | TGE |
| (The signal peptide for | EIRYMLWLSADLKFRIKQKGEYLPLLQGKSLGMIFEKRSTRTRLSTETGFALL |
| mitochondrial import is | GGH |
| underlined*) (SEQ ID | PCFLTTQDIHLGVNESLTDTARVLSSMADAVLARVYKQSDLDTLAKEASIPIIN |
| NO: 4) | GLS |
| | DLYHPIQILADYLTLQEHYSSLKGLTLSWIGDGNNILHSIMMSAAKFGMHLQA |
| | AT |
| | PKGYEPDASVTKLAEQYAKENGTKLLLTNDPLEAAHGGNVLITDTWISMGQE |
| | EEK |
| | KKRLQAFQGYQVTMKTAKVAASDWTFLHCLPRKPEEVDDEVFYSPRSLVFP |
| | EAEN |
| | RKWTIMAVMVSLLTDYSPQLQKPKF |

*The OTC protein comprises a signal peptide which is translated and responsible for translocation to the mitochondria. This signal peptide is represented by the first 32 amino acids as underlined in SEQ ID NO: 3 and SEQ ID NO: 4 (corresponding nucleotide sequence is underlined in SEQ ID NO: 1 and SEQ ID NO: 2). The signal sequence of SEQ ID NO: 4 has also been modified as compared to SEQ ID NO: 3; specifically, an amino acid, valine is inserted at position 3 of SEQ ID NO: 4. This modification provides better mitochondrial localization of the modified OTC of SEQ ID NO: 4 as compared to wild type human OTC of SEQ ID NO: 3.

Preferably, the open reading frame (ORF) or coding sequence (CDS) of an mRNA sequence described herein encodes an amino acid sequence that is substantially identical to the modified OTC protein of SEQ ID NO: 4.

Preferably, the open reading frame (ORF) or coding sequence (CDS) of an mRNA sequence described herein encodes an amino acid sequence that is substantially identical to wild type human OTC protein of SEQ ID NO: 3. Preferably, an OTC protein encoded by an mRNA described herein comprises a protein sequence that is at least about 70%, about 75%, about 80%, about 85%, about 90%, about 95% about 96%, about 97%, about 98%, about 99%, or about 100% identical to a modified human OTC protein of SEQ ID NO: 3 shown in Table 1 while retaining the OTC protein activity of catalyzing the synthesis of citrulline (in the liver and small intestine) from carbamoyl phosphate and ornithine.

Preferably, the ORF or CDS of an mRNA described herein encodes an amino acid sequence that is substantially identical to modified human OTC protein of SEQ ID NO: 4.

Preferably, the ORF or CDS of an mRNA described herein encoding a human OTC protein comprises a codon optimized polynucleotide sequence at least about 70%, about 75%, about 80%, about 85%, about 90%, about 95%, about 96%, about 97%, about 98%, about 99%, or about 100% identical to the mRNA coding sequence of SEQ ID NO: 1 of Table 1.

Preferably an mRNA described herein further comprises a sequence immediately downstream (i.e., in the 3′ direction from) of the CDS that creates a triple stop codon. The triple stop codon may be incorporated to enhance the efficiency of translation. In some embodiments, the translatable oligomer may comprise the sequence AUAAGUGAA (SEQ ID NO: 25) immediately downstream of an OTC CDS of an mRNA sequence described herein.

### Codon Optimization

A polynucleotide sequence encoding a protein can be altered relative to the wild type for the same sequence to select the best combination of codons that code for the amino acids of the protein. For an mRNA, all or a portion of the mRNA, for example, the coding region or open reading frame (ORF), can be optimized with respect to the codons in that region. Codon optimized sequences can increase protein expression levels (Gustafsson et al., Codon bias and heterologous protein expression. 2004, Trends Biotechnol 22:346-53) of the encoded proteins while providing other advantages. Optimization of the codons in a sequence will depend on several characteristics of an mRNA construct including high codon adaptation index (CAI), the Low-U method, mRNA secondary structures, cis-regulatory sequences, GC content and many other similar variables. These variables have been shown to correlate with protein expression levels (Villalobos et al., Gene Designer: a synthetic biology tool for constructing artificial DNA segments. 2006, BMC Bioinformatics 7:285). The high CAI (codon adaptation index) method picks a most frequently used synonymous codon for an entire protein coding sequence. The most frequently used codon for each amino acid is deduced from 74,218 protein-coding genes from a human genome. The Low-U method targets only U-containing codons that can be replaced with a synonymous codon with fewer U moieties. If there are a few choices for the replacement, the more frequently used codon will be selected. The remaining codons in the sequence are not changed by the Low-U method. This method may be used in conjunction with the disclosed mRNAs to design coding sequences that are to be synthesized with, for example, 5-methoxyuridine or N′-methyl pseudouridine. Methods of codon optimization in combination with the use of a modified nucleotide monomer are described in U.S. 2018/0327471, the contents of which are herein incorporated by reference.

In addition, the nucleotide sequence of any region of the mRNA or DNA template may be codon optimized. Codon optimization methods are known in the art and may be useful in efforts to achieve one or more of several goals. These goals include to match codon frequencies in target and host organisms to ensure proper folding, to bias GC nucleotide pair content to increase mRNA stability or reduce secondary structures, to minimize tandem repeat codons or base runs that may impair gene construction or expression, to customize transcriptional and translational control regions, to insert or remove protein trafficking sequences, to remove/add post translation modification sites in encoded protein (e.g. glycosylation sites), to add, remove or shuffle protein domains, to insert or delete restriction sites, to modify ribosome binding sites and mRNA degradation sites, to adjust translational rates to allow the various domains of the protein to fold properly, or to reduce or eliminate problematic secondary structures within the mRNA. Suitable codon optimization tools, algorithms and services are known in the art.

In some embodiments, the nucleotide sequence of any region of the mRNA or DNA templates described herein may be codon optimized. Preferably, the primary cDNA template may include reducing the occurrence or frequency of appearance of certain nucleotides in the template strand. For example, the occurrence of a nucleotide in a template may be reduced to a level below 25% of said nucleotides in the template. In further examples, the occurrence of a nucleotide in a template may be reduced to a level below 20% of said nucleotides in the template. In some examples, the occurrence of a nucleotide in a template may be reduced to a level below 16% of said nucleotides in the template. Preferably, the occurrence of a nucleotide in a template may be reduced to a level below 15%, and preferably may be reduced to a level below 12% of said nucleotides in the template.

In some embodiments, the nucleotide reduced is uridine. For example, the present disclosure provides nucleic acids with altered uracil content wherein at least one codon in the wild-type sequence has been replaced with an alternative codon to generate a uracil-altered sequence. Altered uracil sequences can have at least one of the following properties:
(i) an increase or decrease in global uracil content (i.e., the percentage of uracil of the total nucleotide content in the nucleic acid of a section of the nucleic acid, e.g., the open reading frame);
(ii) an increase or decrease in local uracil content (i.e., changes in uracil content are limited to specific subsequences);
(iii) a change in uracil distribution without a change in the global uracil content;
(iv) a change in uracil clustering (e.g., number of clusters, location of clusters,
or distance between clusters); or
(v) combinations thereof.

In some embodiments, the percentage of uracil nucleobases in the nucleic acid sequence is reduced with respect to the percentage of uracil nucleobases in the wild-type nucleic acid sequence. For example, 30% of nucleobases may be uracil in the wild-type sequence but the nucleobases that are uracil are preferably lower than 15%, preferably lower than 12% and preferably lower than 10% of the nucleobases in the nucleic acid sequences of the disclosure. The percentage uracil content can be determined by dividing the number of uracil in a sequence by the total number of nucleotides and multiplying by 100.

In some embodiments, the percentage of uracil nucleobases in a subsequence of the nucleic acid sequence is reduced with respect to the percentage of uracil nucleobases in the corresponding subsequence of the wild-type sequence. For example, the wild-type sequence may have a 5′-end region (e.g., 30 codons) with a local uracil content of 30%, and the uracil content in that same region could be reduced to preferably 15% or lower, preferably 12% or lower and preferably 10% or lower in the nucleic acid sequences of the disclosure. These subsequences can also be part of the wild-type sequences of the heterologous 5′ and 3′ UTR sequences of the present disclosure.

In some embodiments, codons in the nucleic acid sequence of the disclosure reduce or modify, for example, the number, size, location, or distribution of uracil clusters that could have deleterious effects on protein translation. Although lower uracil content is desirable in certain aspects, the uracil content, and in particular the local uracil content, of some subsequences of the wild-type sequence can be greater than the wild-type sequence and still maintain beneficial features (e.g., increased expression).

In some embodiments, the uracil-modified sequence induces a lower Toll-Like Receptor (TLR) response when compared to the wild-type sequence. Several TLRs recognize and respond to nucleic acids. Double-stranded (ds) RNA, a frequent viral constituent, has been shown to activate TLR3. Single-stranded (ss) RNA activates TLR7. RNA oligonucleotides, for example RNA with phosphorothioate internucleotide linkages, are ligands of human TLR8. DNA containing unmethylated CpG motifs, characteristic of bacterial and viral DNA, activate TLR9.

As used herein, the term “TLR response” is defined as the recognition of single-stranded RNA by a TLR7 receptor, and preferably encompasses the degradation of the RNA and/or physiological responses caused by the recognition of the single-stranded RNA by the receptor. Methods to determine and quantify the binding of an RNA to a TLR7 are known in the art. Similarly, methods to determine whether an RNA has triggered a TLR7-mediated physiological response (e.g., cytokine secretion) are well known in the art. In some embodiments, a TLR response can be mediated by TLR3, TLR8, or TLR9 instead of TLR7. Suppression of TLR7-mediated response can be accomplished via nucleoside modification. RNA undergoes over a hundred different nucleoside modifications in nature. Human rRNA, for example, has ten times more pseudouracil (′P) and 25 times more 2′-O-methylated nucleosides than bacterial rRNA. Bacterial mRNA contains no nucleoside modifications, whereas mammalian mRNAs have modified nucleosides such as 5-methylcytidine (m⁵° C.), N⁶-methyladenosine (m⁶A), inosine and many 2′-O-methylated nucleosides in addition to N⁷-methylguanosine (m⁷G).

In some embodiments, the uracil content of polynucleotides disclosed herein and preferably polynucleotides encoding the modified OTC protein of SEQ ID NO: 4 is less than about 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% of the total nucleobases in the sequence in the reference sequence. In some embodiments, the uracil content of polynucleotides disclosed herein and preferably polynucleotides encoding the modified OTC protein of SEQ ID NO: 4, is between about 5% and about 25%. In some embodiments, the uracil content of polynucleotides disclosed herein and preferably polynucleotides encoding the modified OTC protein of SEQ ID NO: 4 is between about 15% and about 25%.

### Natural and Modified Nucleotides

Preferably an mRNA described herein comprises one or more chemically modified nucleotides. Examples of nucleic acid monomers include non-natural, modified, and chemically-modified nucleotides, including any such nucleotides known in the art. Nucleotides can be artificially modified at either the base portion or the sugar portion. In nature, most polynucleotides comprise nucleotides that are “unmodified” or “natural” nucleotides, which include the purine bases adenine (A) and guanine (G), and the pyrimidine bases thymine (T), cytosine (C) and uracil (U). These bases are typically fixed to a ribose or deoxy ribose at the 1′ position. The use of mRNA polynucleotides comprising chemically modified nucleotides have been shown to improve mRNA expression, expression rates, half-life and/or expressed protein concentrations. mRNA polynucleotides comprising chemically modified nucleotides have also been useful in optimizing protein localization thereby avoiding deleterious bio-responses such as immune responses and/or degradation pathways.

Examples of modified or chemically-modified nucleotides include 5-hydroxycytidines, 5-alkylcytidines, 5-hydroxyalkylcytidines, 5-carboxycytidines, 5-formylcytidines, 5-alkoxycytidines, 5-alkynylcytidines, 5-halocytidines, 2-thiocytidines, N⁴-alkylcytidines, N⁴-aminocytidines, N⁴-acetylcytidines, and N⁴,N⁴-dialkylcytidines.

Examples of modified or chemically-modified nucleotides include 5-hydroxycytidine, 5-methylcytidine, 5-hydroxymethylcytidine, 5-carboxycytidine, 5-formylcytidine, 5-methoxycytidine, 5-propynylcytidine, 5-bromocytidine, 5-iodocytidine, 2-thiocytidine; N⁴-methylcytidine, N⁴-aminocytidine, N⁴-acetylcytidine, and N⁴,N⁴-dimethylcytidine.

Examples of modified or chemically-modified nucleotides include 5-hydroxyuridines, 5-alkyluridines, 5-hydroxyalkyluridines, 5-carboxyuridines, 5-carboxyalkylesteruridines, 5-formyluridines, 5-alkoxyuridines, 5-alkynyluridines, 5-halouridines, 2-thiouridines, and 6-alkyluridines.

Examples of modified or chemically-modified nucleotides include 5-hydroxyuridine, 5-methyluridine, 5-hydroxymethyluridine, 5-carboxyuridine, 5-carboxymethylesteruridine, 5-formyluridine, 5-methoxyuridine (also referred to herein as “5MeOU”), 5-propynyluridine, 5-bromouridine, 5-fluorouridine, 5-iodouridine, 2-thiouridine, and 6-methyluridine.

Examples of modified or chemically-modified nucleotides include 5-methoxycarbonylmethyl-2-thiouridine, 5-methylaminomethyl-2-thiouridine, 5-carbamoylmethyluridine, 5-carbamoylmethyl-2′-O-methyluridine, 1-methyl-3-(3-amino-3-carboxypropy) pseudouridine, 5-methylaminomethyl-2-selenouridine, 5-carboxymethyluridine, 5-methyldihydrouridine, 5-taurinomethyluridine, 5-taurinomethyl-2-thiouridine, 5-(isopentenylaminomethyl) uridine, 2′-O-methylpseudouridine, 2-thio-2′O-methyluridine, and 3,2′-O-dimethyluridine.

Examples of modified or chemically-modified nucleotides include N⁶-methyladenosine, 2-aminoadenosine, 3-methyladenosine, 8-azaadenosine, 7-deazaadenosine, 8-oxoadenosine, 8-bromoadenosine, 2-methylthio-N⁶-methyladenosine, N⁶-isopentenyladenosine, 2-methylthio-N⁶-isopentenyladenosine, N⁶-(cis-hydroxyisopentenyl) adenosine, 2-methylthio-N⁶-(cis-hydroxyisopentenyl) adenosine, N⁶-glycinylcarbamoyladenosine, N⁶-threonylcarbamoyl-adenosine, N⁶-methyl-N⁶-threonylcarbamoyl-adenosine, 2-methylthio-N⁶-threonylcarbamoyl-adenosine, N⁶,N⁶-dimethyladenosine, N⁶-hydroxynorvalylcarbamoyladenosine, 2-methylthio-N⁶-hydroxynorvalylcarbamoyl-adenosine, N⁶-acetyl-adenosine, 7-methyl-adenine, 2-methylthio-adenine, 2-methoxy-adenine, alpha-thio-adenosine, 2′-O-methyl-adenosine, N⁶,2′—O-dimethyl-adenosine, N⁶,N⁶,2′—O-trimethyl-adenosine, 1,2′-O-dimethyl-adenosine, 2′-O-ribosyladenosine, 2-amino-N⁶-methyl-purine, 1-thio-adenosine, 2′-F-ara-adenosine, 2′-F-adenosine, 2′-OH-ara-adenosine, and N⁶-(19-amino-pentaoxanonadecyl)-adenosine.

Examples of modified or chemically-modified nucleotides include N¹-alkylguanosines, N²-alkylguanosines, thienoguanosines, 7-deazaguanosines, 8-oxoguanosines, 8-bromoguanosines, 06-alkylguanosines, xanthosines, inosines, and N¹-alkylinosines.

Examples of modified or chemically-modified nucleotides include N¹-methylguanosine, N²-methylguanosine, thienoguanosine, 7-deazaguanosine, 8-oxoguanosine, 8-bromoguanosine, 06-methylguanosine, xanthosine, inosine, and N¹-methylinosine.

Examples of modified or chemically-modified nucleotides include pseudouridines. Examples of pseudouridines include N¹-alkylpseudouridines, N¹ cycloalkylpseudouridines, N¹-hydroxypseudouridines, N¹-hydroxyalkylpseudouridines, N¹-phenylpseudouridines, N¹-phenylalkylpseudouridines, N¹-aminoalkylpseudouridines, N³-alkylpseudouridines, N⁶-alkylpseudouridines, N⁶-alkoxypseudouridines, N⁶-hydroxypseudouridines, N⁶-hydroxyalkylpseudouridines, N⁶-morpholinopseudouridines, N⁶-phenylpseudouridines, and N⁶-halopseudouridines. Examples of pseudouridines include N¹-alkyl-N⁶-alkylpseudouridines, N¹-alkyl-N⁶-alkoxypseudouridines, N¹-alkyl-N⁶-hydroxypseudouridines, N¹-alkyl-N⁶-hydroxyalkylpseudouridines, N¹-alkyl-N⁶-morpholinopseudouridines, N¹-alkyl-N⁶-phenylpseudouridines, and N¹-alkyl-N⁶-halopseudouridines. In these examples, the alkyl, cycloalkyl, and phenyl substituents may be unsubstituted, or further substituted with alkyl, halo, haloalkyl, amino, or nitro substituents.

Examples of pseudouridines include N¹-methylpseudouridine (also referred to herein as “N1MPU”), N¹-ethylpseudouridine, N¹-propylpseudouridine, N¹ cyclopropylpseudouridine, N¹-phenylpseudouridine, N¹-aminomethylpseudouridine, N³-methylpseudouridine, N¹-hydroxypseudouridine, and N¹-hydroxymethylpseudouridine.

Examples of nucleic acid monomers include modified and chemically-modified nucleotides, including any such nucleotides known in the art.

Examples of modified and chemically-modified nucleotide monomers include any such nucleotides known in the art, for example, 2′-O-methyl ribonucleotides, 2′-O-methyl purine nucleotides, 2′-deoxy-2′-fluoro ribonucleotides, 2′-deoxy-2′-fluoro pyrimidine nucleotides, 2′-deoxy ribonucleotides, 2′-deoxy purine nucleotides, universal base nucleotides, 5-C-methyl-nucleotides, and inverted deoxyabasic monomer residues.

Examples of modified and chemically-modified nucleotide monomers include 3′-end stabilized nucleotides, 3′-glyceryl nucleotides, 3′-inverted abasic nucleotides, and 3′-inverted thymidine.

Examples of modified and chemically-modified nucleotide monomers include locked nucleic acid nucleotides (LNA), 2′-0,4′-C-methylene-(D-ribofuranosyl) nucleotides, 2′-methoxyethoxy (MOE) nucleotides, 2′-methyl-thio-ethyl, 2′-deoxy-2′-fluoro nucleotides, and 2′-O-methyl nucleotides. In an exemplary embodiment, the modified monomer is a locked nucleic acid nucleotide (LNA).

Examples of modified and chemically-modified nucleotide monomers include 2′,4′-constrained 2′-O-methoxyethyl (cMOE) and 2′-O-Ethyl (cEt) modified DNAs.

Examples of modified and chemically-modified nucleotide monomers include 2′-amino nucleotides, 2′-O-amino nucleotides, 2′-C-allyl nucleotides, and 2′-O-allyl nucleotides.

Examples of modified and chemically-modified nucleotide monomers include N⁶-methyladenosine nucleotides.

Examples of modified and chemically-modified nucleotide monomers include nucleotide monomers with modified bases 5-(3-amino) propyluridine, 5-(2-mercapto) ethyluridine, 5-bromouridine; 8-bromoguanosine, or 7-deazaadenosine.

Examples of modified and chemically-modified nucleotide monomers include 2′-O-aminopropyl substituted nucleotides.

Examples of modified and chemically-modified nucleotide monomers include replacing the 2′—OH group of a nucleotide with a 2′-R, a 2′—OR, a 2′-halogen, a 2′—SR, or a 2′-amino, where R can be H, alkyl, alkenyl, or alkynyl.

Example of base modifications described above can be combined with additional modifications of nucleoside or nucleotide structure, including sugar modifications and linkage modifications. Certain modified or chemically-modified nucleotide monomers may be found in nature.

Preferred nucleotide modifications include N¹-methylpseudouridine and 5-methoxyuridine.

### Untranslated Region (UTR)

In molecular genetics, an untranslated region (UTR) refers to either of two sections, one on each side of a coding sequence on a strand of mRNA. If it is found on the 5′ side, it is called the 5′ UTR (or leader sequence), or if it is found on the 3′ side, it is called the 3′ UTR (or trailer sequence). As an mRNA is translated into a protein in vivo, several regions of the mRNA are usually not translated, including the 5′ and 3′ UTRs. In some embodiments, an mRNA described herein further comprises a 5′ untranslated region (UTR) sequence. The 5′ UTR is upstream from the coding sequence. Within the 5′ UTR is a sequence that is recognized by the ribosome which allows the ribosome to bind and initiate translation. In contrast, the 3′ UTR is typically found immediately following the translation stop codon of the coding region. The 3′ UTR can play an important role in translation termination as well as post-transcriptional modification. Thus, as is understood in the art, the 5′ and/or 3′ UTR may affect an mRNA's stability or efficiency of translation. The 5′ UTR may be derived from an mRNA molecule known in the art as relatively stable (e.g., histone, tubulin, globin, glyceraldehyde 1-phosphate dehydrogenase (GAPDH), actin, or citric acid cycle enzymes) to increase the stability of the translatable oligomer. In other embodiments, a 5′ UTR sequence may include a partial sequence of a cytomegalovirus (CMV) immediate-early 1 (IE1) gene.

In some embodiments, the 5′ UTR comprises a sequence selected from the 5′ UTRs of human IL-6, alanine aminotransferase 1, human apolipoprotein E, human fibrinogen alpha chain, human transthyretin, human haptoglobin, human alpha-1-antichymotrypsin, human antithrombin, human alpha-1-antitrypsin, human albumin, human beta globin, human complement C3, human complement C5, SynK (thylakoid potassium channel protein derived from the cyanobacteria, Synechocystis sp.), mouse beta globin, mouse albumin, and a tobacco etch virus, or fragments of any of the foregoing. Preferably, the 5′ UTR is derived from a tobacco etch virus (TEV). Preferably, an mRNA described herein comprises a 5′ UTR sequence that is derived from a gene expressed by Arabidopsis thaliana. Preferably, the 5′ UTR sequence of a gene expressed by Arabidopsis thaliana is AT1G58420. Examples of 5 UTRs and 3′ UTRs are described in PCT/US2018/035419, the contents of which are herein incorporated by reference. Preferred 5′ UTR sequences comprise SEQ ID NOs: 5-10, 125-127 and 230-250: as shown in Table 2.

TABLE 2
5′UTR sequences

| Name | Sequence | Seq ID No.: |
|---|---|---|
| EV | UCAACACAACAUAUACAAAACAAACGAAUCUCAAGCAAUCAAGC | SEQ ID NO: 5 |
| | AUUCUACUUCUAUUGCAGCAAUUUAAAUCAUUUCUUUUAAAGC | |
| | AAAAGCAAUUUUCUGAAAAUUUUCACCAUUUACGAACGAUAG | |
| AT1G58420 | AUUAUUACAUCAAAACAAAAAGCCGCCA | SEQ ID NO: 6 |
| ARC5-2 | CUUAAGGGGGCGCUGCCUACGGAGGUGGCAGCCAUCUCCUUCUC | SEQ ID NO: 7 |
| | GGCAUCAAGCUUACCAUGGUGCCCCAGGCCCUGCUCUUGGUCCC | |
| | GCUGCUGGUGUUCCCCCUCUGCUUCGGCAAGUUCCCCAUCUACA | |
| | CCAUCCCCGACAAGCUGGGGCCGUGGAGCCCCAUCGACAUCCAC | |
| | CACCUGUCCUGCCCCAACAACCUCGUGGUCGAGGACGAGGGCUG | |
| | CACCAACCUGAGCGGGUUCUCCUAC | |
| HCV | UGAGUGUCGU ACAGCCUCCA GGCCCCCCCC UCCCGGGAGA | SEQ ID NO: 8 |
| | GCCAUAGUGG UCUGCGGAACCGGUGAGUAC ACCGGAAUUG | |
| | CCGGGAAGAC UGGGUCCUUU CUUGGAUAAA | |
| | CCCACUCUAUGCCCGGCCAU UUGGGCGUGC CCCCGCAAGA | |
| | CUGCUAGCCG AGUAGUGUUG GGUUGCG | |
| HUMAN | AAUUAUUGGUUAAAGAAGUAUAUUAGUGCUAAUUUCCCUCCGU | SEQ ID NO: 9 |
| ALBUMIN | UUGUCCUAGCUUUUCUCUUCUGUCAACCCCACACGCCUUUGGCA | |
| | CA | |
| EMCV | CUCCCUCCCC CCCCCCUAAC GUUACUGGCC GAAGCCGCUU | SEQ ID NO: 10 |
| | GGAAUAAGGC CGGUGUGCGU UUGUCUAUAU GUUAUUUUCC | |
| | ACCAUAUUGC CGUCUUUUGG CAAUGUGAGG GCCCGGAAAC | |
| | CUGGCCCUGU CUUCUUGACG AGCAUUCCUA GGGGUCUUUC | |
| | CCCUCUCGCC AAAGGAAUGC AAGGUCUGUU GAAUGUCGUG | |
| | AAGGAAGCAG UUCCUCUGGA AGCUUCUUGA AGACAAACAA | |
| | CGUCUGUAGC GACCCUUUGC AGGCAGCGGA ACCCCCCACC | |
| | UGGCGACAGG UGCCUCUGCG GCCAAAAGCC ACGUGUAUAA | |
| | GAUACACCUG CAAAGGCGGC ACAACCCCAG UGCCACGUUG | |
| | UGAGUUGGAU AGUUGUGGAA AGAGUCAAAU GGCUCUCCUC | |
| | AAGCGUAUUC AACAAGGGGC UGAAGGAUGC CCAGAAGGUA | |
| | CCCCAUUGUA UGGGAUCUGA UCUGGGGCCU CGGUGCACAU | |
| | GCUUUACGUG UGUUUAGUCG AGGUUAAAAA ACGUCUAGGC | |
| | CCCCCGAACC ACGGGGACGU GGUUUUCCUU UGAAAAACAC | |
| | GAUGAUAAU | |
| AT1G67090 | CACAAAGAGUAAAGAAGAACA | SEQ ID NO: 125 |
| AT1G35720 | AACACUAAAAGUAGAAGAAAA | SEq ID NO: 126 |
| AT5G45900 | CUCAGAAAGAUAAGAUCAGCC | SEQ ID NO: 127 |
| AT5G61250 | AACCAAUCGAAAGAAACCAAA | SEQ ID NO: 230 |
| AT5G46430 | CUCUAAUCACCAGGAGUAAAA | SEQ ID NO: 231 |
| AT5G47110 | GAGAGAGAUCUUAACAAAAAA | SEQ ID NO: 232 |
| AT1G03110 | UGUGUAACAACAACAACAACA | SEQ ID NO: 233 |
| AT3G12380 | CCGCAGUAGGAAGAGAAAGCC | SEQ ID NO: 234 |
| AT5G45910 | AAAAAAAAAAGAAAUCAUAAA | SEQ ID NO: 235 |
| AT1G07260 | GAGAGAAGAAAGAAGAAGACG | SEQ ID NO: 236 |
| AT3G55500 | CAAUUAAAAAUACUUACCAAA | SEQ ID NO: 237 |
| AT3G46230 | GCAAACAGAGUAAGCGAAACG | SEQ ID NO: 238 |
| AT2G36170 | GCGAAGAAGACGAACGCAAAG | SEQ ID NO: 239 |
| AT1G10660 | UUAGGACUGUAUUGACUGGCC | SEQ ID NO: 240 |
| AT4G14340 | AUCAUCGGAAUUCGGAAAAAG | SEQ ID NO: 241 |
| AT1G49310 | AAAACAAAAGUUAAAGCAGAC | SEQ ID NO: 242 |
| AT4G14360 | UUUAUCUCAAAUAAGAAGGCA | SEQ ID NO: 243 |
| AT1G28520 | GGUGGGGAGGUGAGAUUUCUU | SEQ ID NO: 244 |
| AT1G20160 | UGAUUAGGAAACUACAAAGCC | SEQ ID NO: 245 |
| AT5G37370 | CAUUUUUCAAUUUCAUAAAAC | SEQ ID NO: 246 |
| AT4G11320 | UUACUUUUAAGCCCAACAAAA | SEQ ID NO: 247 |
| AT5G40850 | GGCGUGUGUGUGUGUUGUUGA | SEQ ID NO: 248 |
| AT1G06150 | GUGGUGAAGGGGAAGGUUUAG | SEQ ID NO: 249 |
| AT2G26080 | UUGUUUUUUUUUGGUUUGGUU | SEQ ID NO: 250 |

In some embodiments, the 5′UTR sequence comprises SEQ ID NO: 6 (AT1G58420).

In some embodiments, an mRNA described herein comprises a 3′UTR. In some embodiments, the 3′ UTR comprises a sequence selected from the 3′ UTRs of alanine aminotransferase 1, human apolipoprotein E, human fibrinogen alpha chain, human haptoglobin, human antithrombin, human alpha globin, human beta globin, human complement C3, human growth factor, human hepcidin, MALAT-1, mouse beta globin, mouse albumin, and Xenopus beta globin, or fragments of any of the foregoing. In some embodiments, the 3′ UTR is derived from Xenopus beta globin. Exemplary 3′ UTR sequences include SEQ ID NOs: 16-22 as shown in Table 3.

TABLE 3
3′UTR sequences

| Name | Sequence | Seq ID No.: |
|---|---|---|
| XBG | CUAGUGACUGACUAGGAUCUGGUUACCACUAAACCAGCCUC | SEQ ID NO: 16 |
| | AAGAACACCCGAAUGGAGUCUCUAAGCUACAUAAUACCAA | |
| | CUUACACUUACAAAAUGUUGUCCCCCAAAAUGUAGCCAUUC | |
| | GUAUCUGCUCCUAAUAAAAAGAAAGUUUCUUCACAU | |
| HUMAN | UGCAAGGCUGGCCGGAAGCCCUUGCCUGAAAGCAAGAUUU | SEQ ID NO: 17 |
| HAPTOGLOBIN | CAGCCUGGAAGAGGGCAAAGUGGACGGGAGUGGACAGGAG | |
| | UGGAUGCGAUAAGAUGUGGUUUGAAGCUGAUGGGUGCCAG | |
| | CCCUGCAUUGCUGAGUCAAUCAAUAAAGAGCUUUCUUUUG | |
| | ACCCAU | |
| HUMAN | ACGCCGAAGCCUGCAGCCAUGCGACCCCACGCCACCCCGUG | SEQ ID NO: 18 |
| APOLIPOPROTEIN | CCUCCUGCCUCCGCGCAGCCUGCAGCGGGAGACCCUGUCCC | |
| E | CGCCCCAGCCGUCCUCCUGGGGUGGACCCUAGUUUAAUAAA | |
| | GAUUCACCAAGUUUCACGCA | |
| HCV | UAGAGCGGCAAACCCUAGCUACACUCCAUAGCUAGUUUCUU | SEQ ID NO: 19 |
| | UUUUUUUUGUUUUUUUUUUUUUUUUUUUUUUUUUUUUUUU | |
| | UUUUUUUUCCUUUCUUUUCCUUCUUUUUUUCCUCUUUUCU | |
| | UGGUGGCUCCAUCUUAGCCCUAGUCACGGCUAGCUGUGAA | |
| | AGGUCCGUGAGCCGCAUGACUGCAGAGAGUGCCGUAACUG | |
| | GUCUCUCUGCAGAUCAUGU | |
| MOUSE ALBUMIN | ACACAUCACAACCACAACCUUCUCAGGCUACCCUGAGAAAA | SEQ ID NO: 20 |
| | AAAGACAUGAAGACUCAGGACUCAUCUUUUCUGUUGGUGU | |
| | AAAAUCAACACCCUAAGGAACACAAAUUUCUUUAAACAUU | |
| | UGACUUCUUGUCUCUGUGCUGCAAUUAAUAAAAAAUGGAA | |
| | AGAAUCUAC | |
| HUMAN ALPHA | GCUGGAGCCUCGGUAGCCGUUCCUCCUGCCCGCUGGGCCUC | SEQ ID NO: 21 |
| GLOBIN | CCAACGGGCCCUCCUCCCCUCCUUGCACCGGCCCUUCCUGG | |
| | UCUUUGAAUAAAGUCUGAGUGGGCAGCA | |
| EMCV | UAGUGCAGUCAC UGGCACAACG CGUUGCCCGG | SEQ ID NO: 22 |
| | UAAGCCAAUC GGGUAUACAC GGUCGUCAUACUGCAGACAG | |
| | GGUUCUUCUA CUUUGCAAGA UAGUCUAGAG | |
| | UAGUAAAAUA AAUAGUAUAAG | |

### Triple Stop Codon

In some embodiments, the translatable oligomer encoding OTC may comprise a sequence immediately downstream of a coding region (i.e., ORF) that creates a triple stop codon. A triple stop codon is a sequence of three consecutive stop codons. The triple stop codon can ensure total insulation of an expression cassette and may be incorporated to enhance the efficiency of translation. In some embodiments, the mRNA may comprise a triple combination of any of the sequences UAG, UGA, or UAA immediately downstream of a ORF described herein. The triple combination can be three of the same codons, three different codons, or any other permutation of the three stop codons.

### Translation Enhancers and Kozak Sequences

For translation initiation, proper interactions between ribosomes and mRNAs must be established to determine the exact position of the translation initiation region. However, ribosomes also must dissociate from the translation initiation region to slide toward the downstream sequence during mRNA translation. Translation enhancers upstream from initiation sequences of mRNAs enhance the yields of protein biosynthesis. Several studies have investigated the effects of translation enhancers. In some embodiments, an mRNA described herein comprises a translation enhancer sequence. These translation enhancer sequences enhance the translation efficiency of an mRNA described herein and thereby provide increased production of the protein encoded by the mRNA. The translation enhancer region may be located in the 5′ or 3′ UTR of an mRNA sequence. Examples of translation enhancer regions include naturally-occurring enhancer regions from the TEV 5′ UTR and the Xenopus beta-globin 3′ UTR. Exemplary 5′ UTR enhancer sequences include but are not limited to those derived from mRNAs encoding human heat shock proteins (HSP) including HSP70-P2, HSP70-M1 HSP72-M2, HSP17.9 and HSP70-P1. Preferred translation enhancer sequences used in accordance with the embodiments of the present disclosure are represented by SEQ ID Nos: 11-15 as shown in Table 4.

TABLE 4
5′UTR Enhancers

| Name | Sequence | Seq ID No.: |
|---|---|---|
| HSP70-P2 | GUCAGCUUUCAAACUCUUUGUUUCUUGUUUGUUGAUUGAGAAUA | SEQ ID NO: 11 |
| HSP70-M1 | CUCUCGCCUGAGAAAAAAAAUCCACGAACCAAUUUCUCAGCAACCA | SEQ ID NO: 12 |
| | GCAGCACG | |
| HSP72-M2 | ACCUGUGAGGGUUCGAAGGAAGUAGCAGUGUUUUUUGUUCCUAGAG | SEQ ID NO: 13 |
| | GAAGAG | |
| HSP17.9 | ACACAGAAACAUUCGCAAAAACAAAAUCCCAGUAUCAAAAUUCUUC | SEQ ID NO: 14 |
| | UCUUUUUUUCAUAUUUCGCAAAGAC | |
| HSP70-P1 | CAGAAAAAUUUGCUACAUUGUUUCACAAACUUCAAAUAUUAUUCAU | SEQ ID NO: 15 |
| | UUAUUU | |

In some embodiments, an mRNA described herein comprises a Kozak sequence. As is understood in the art, a Kozak sequence is a short consensus sequence centered around the translational initiation site of eukaryotic mRNAs that allows for efficient initiation of translation of the mRNA. See, for example, Kozak, Marilyn (1988) Mol. and Cell Biol, 8:2737-2744; Kozak, Marilyn (1991) J. Biol. Chem, 266:19867-19870; Kozak, Marilyn (1990) Proc Natl. Acad. Sci. USA, 87:8301-8305; and Kozak, Marilyn (1989) J. Cell Biol, 108:229-241. It ensures that a protein is correctly translated from the genetic message, mediating ribosome assembly and translation initiation. The ribosomal translation machinery recognizes the AUG initiation codon in the context of the Kozak sequence. A Kozak sequence may be inserted upstream of the coding sequence for OTC, downstream of a 5′ UTR or inserted upstream of the coding sequence for OTC and downstream of a 5′ UTR. In some embodiments, an mRNA described herein comprises a Kozak sequence having the sequence GCCACC (SEQ ID NO: 23). Preferably an mRNA described herein comprises a partial Kozak sequence “p” having the sequence GCCA (SEQ ID NO: 24).

### 5′ Cap

A Cap structure on the 5′-end of mRNAs, which is present in all eukaryotic organisms (and some viruses) is important for stabilizing mRNAs in vivo. Naturally occurring Cap structures comprise a ribo-guanosine residue that is methylated at position N⁷ of the guanine base. This 7-methylguanosine (m⁷G) is linked via a 5′- to 5′-triphosphate chain at the 5′-end of the mRNA molecule. The presence of the m⁷Gppp fragment on the 5′-end is essential for mRNA maturation as it protects the mRNAs from degradation by exonucleases, facilitates transport of mRNAs from the nucleus to the cytoplasm and plays a key role in assembly of the translation initiation complex (Cell 9:645-653, (1976); Nature 266:235, (1977); Federation of Experimental Biologists Society Letter 96:1-11, (1978); Cell 40:223-24, (1985); Prog. Nuc. Acid Res. 35:173-207, (1988); Ann. Rev. Biochem. 68:913-963, (1999); and J Biol. Chem. 274:30337-3040, (1999)).

Only those mRNAs that carry the Cap structure are active in Cap dependent translation; “decapitation” of mRNA results in an almost complete loss of their template activity for protein synthesis (Nature, 255:33-37, (1975); J. Biol. Chem., vol. 253:5228-5231, (1978); and Proc. Natl. Acad. Sci. USA, 72:1189-1193, (1975)).

Another element of eukaryotic mRNA is the presence of 2′-O-methyl nucleoside residues at transcript position 1 (Cap 1), and in some cases, at transcript positions 1 and 2 (Cap 2). The 2′-O-methylation of mRNA provides higher efficacy of mRNA translation in vivo (Proc. Natl. Acad. Sci. USA, 77:3952-3956 (1980)) and further improves nuclease stability of the 5′-capped mRNA. The mRNA with Cap 1 (and Cap 2) is a distinctive mark that allows cells to recognize the bona fide mRNA 5′ end, and in some instances, to discriminate against transcripts emanating from infectious genetic elements (Nucleic Acid Research 43:482-492 (2015)).

Some examples of 5′ cap structures and methods for preparing mRNAs comprising the same are given in WO2015/051169A2, WO/2015/061491, US 2018/0273576, and U.S. Pat. Nos. 8,093,367, 8,304,529, and 10,487,105. In some embodiments, the 5′ cap is m⁷GpppAmpG, which is known in the art. In some embodiments, the 5′ cap is m⁷GpppG or m⁷GpppGm, which are known in the art. Structural formulas for embodiments of 5′ cap structures are provided below.

In some embodiments, an mRNA described herein comprises a 5′ cap having the structure of Formula (Cap I).

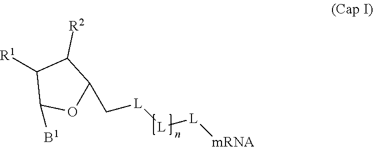

wherein B¹ is a natural or modified nucleobase; R¹ and R² are each independently selected from a halogen, OH, and OCH₃; each L is independently selected from the group consisting of phosphate, phophorothioate, and boranophosphate wherein each L is linked by diester bonds; n is 0 or 1, and mRNA represents an mRNA of the present disclosure linked at its 5′ end. In some embodiments B¹ is G, m⁷G, or A. In some embodiments, n is 0. In some embodiments n is 1. In some embodiments, B¹ is A or m⁶A and R¹ is OCH₃; wherein G is guanine, m⁷G is 7-methylguanine, A is adenine, and m⁶A is N⁶-methyladenine.

In some embodiments, an mRNA described herein comprises a 5′ cap having the structure of Formula (Cap II).

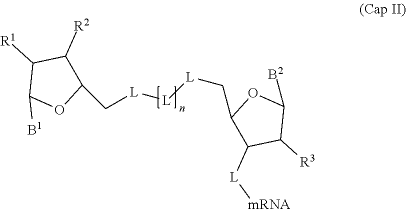

wherein B¹ and B² are each independently a natural or modified nucleobase; R¹, R², and R³ are each independently selected from a halogen, OH, and OCH₃; each L is independently selected from the group consisting of phosphate, phophorothioate, and boranophosphate wherein each L is linked by diester bonds; mRNA represents an mRNA of the present disclosure linked at its 5′ end; and n is 0 or 1. In some embodiments B¹ is G, m⁷G, or A. In some embodiments, n is 0. In some embodiments, n is 1. In some embodiments, B¹ is A or m⁶A and R¹ is OCH₃; wherein G is guanine, m⁷G is 7-methylguanine, A is adenine, and m⁶A is N⁶-methyladenine.

In some embodiments, an mRNA described herein comprises a 5′ cap having the structure of Formula (Cap III).

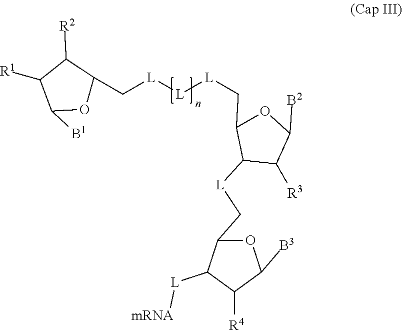

wherein B¹, B², and B³ are each independently a natural or modified nucleobase; R¹, R², R³, and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², R³, and R⁴ is OH. In some embodiments B¹ is G, m⁷G, or A. In some embodiments, B¹ is A or m⁶A and R¹ is OCH₃; wherein G is guanine, m⁷G is 7-methylguanine, A is adenine, and m⁶A is N⁶-methyladenine. In some embodiments, n is 1.

In some embodiments, an mRNA described herein comprises a m⁷GpppG 5′ cap analog having the structure of Formula (Cap IV).

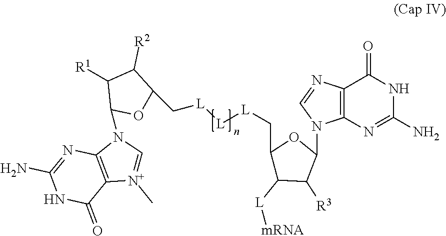

wherein, R¹, R², and R³ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; n is 0 or 1. In some embodiments, at least one of R¹, R², and R³ is OH. In some embodiments, the 5′ cap is m⁷GpppG wherein R¹, R², and R³ are each OH, n is 1, and each L is a phosphate. In some embodiments, n is 1. In some embodiments, the 5′ cap is m⁷GpppGm, wherein R¹ and R² are each OH, R³ is OCH₃, each L is a phosphate, mRNA is the mRNA encoding an enzyme having OTC activity linked at its 5′ end, and n is 1.

In some embodiments, an mRNA described herein comprises a m⁷Gpppm⁷G 5′ cap analog having the structure of Formula (Cap V).

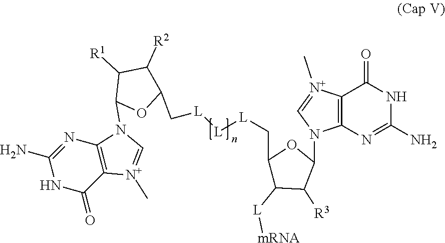

wherein, R¹, R², and R³ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R³ is OH. In some embodiments, n is 1.

In some embodiments, an mRNA described herein comprises a m⁷Gpppm⁷GpN, 5′ cap analog, wherein N is a natural or modified nucleotide, the 5′ cap analog having the structure of Formula (Cap VI).

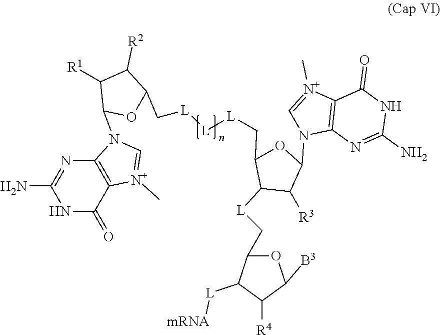

wherein B³ is a natural or modified nucleobase; R¹, R², R³, and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 3. In some embodiments, at least one of R¹, R², R³, and R⁴ is OH. In some embodiments B¹ is G, m⁷G, or A. In some embodiments, B¹ is A or m⁶A and R¹ is OCH₃; wherein G is guanine, m⁷G is 7-methylguanine, A is adenine, and m⁶A is N⁶-methyladenine. In some embodiments, n is 1.

In some embodiments, an mRNA described herein comprises a m⁷Gpppm⁷GpG 5′ cap analog having the structure of Formula (Cap VII).

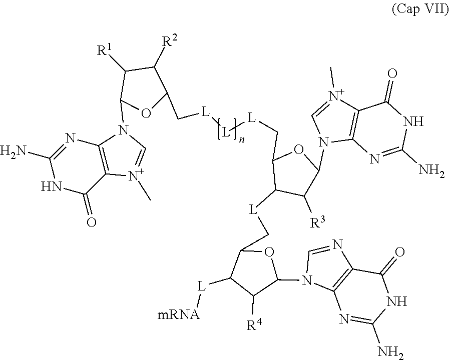

wherein, R¹, R², R³, and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², R³, and R⁴ is OH. In some embodiments, n is 1.

In some embodiments, an mRNA described herein comprises a m⁷Gpppm⁷Gpm⁷G 5′ cap analog having the structure of Formula (Cap VIII).

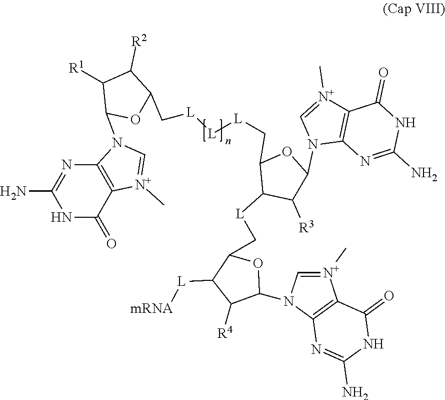

wherein, R¹, R², R³, and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; n is 0 or 1. In some embodiments, at least one of R¹, R², R³, and R⁴ is OH. In some embodiments, n is 1.

In some embodiments, an mRNA described herein comprises a m⁷GpppA 5′ cap analog having the structure of Formula (Cap IX).

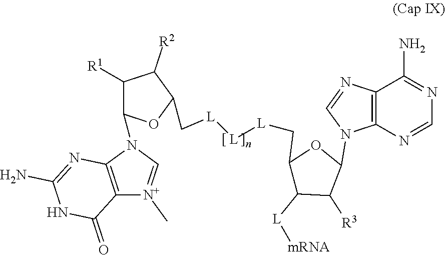

wherein, R¹, R², and R³ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R³ is OH. In some embodiments, n is 1.

In some embodiments, an mRNA described herein comprises a m⁷GpppApN 5′ cap analog, wherein N is a natural or modified nucleotide, and the 5′ cap has the structure of Formula (Cap X).

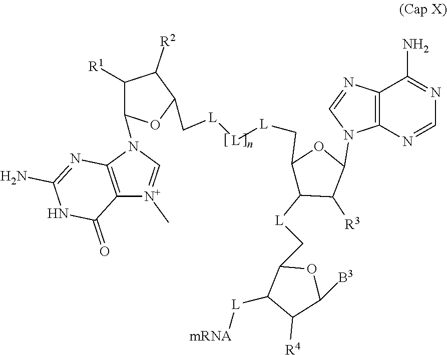

wherein B³ is a natural or modified nucleobase; R¹, R², R³, and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², R³, and R⁴ is OH. In some embodiments B³ is G, m⁷G, A or m⁶A; wherein G is guanine, m⁷G is 7-methylguanine, A is adenine, and m⁶A is N⁶-methyladenine. In some embodiments, n is 1.

In some embodiments, an mRNA described herein comprises a m⁷Gppp AmpG 5′ cap analog having the structure of Formula (Cap XI).

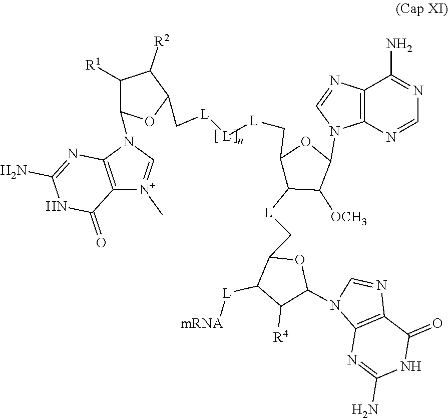

wherein, R¹, R², and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R⁴ is OH. In some embodiments, the compound of Formula Cap XI is m⁷GpppAmpG, wherein R¹, R², and R⁴ are each OH, n is 1, and each L is a phosphate linkage. In some embodiments, n is 1.

In some embodiments, an mRNA described herein comprises a m⁷GpppApm⁷G 5′ cap analog having the structure of Formula (Cap XII).

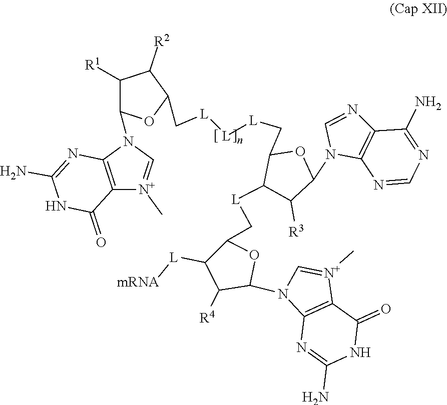

wherein, R¹, R², R³, and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², R³, and R⁴ is OH. In some embodiments, n is 1.

In some embodiments, an mRNA described herein comprises m⁷GpppAmpm⁷G 5′ cap analog having the structure of Formula (Cap XIII).

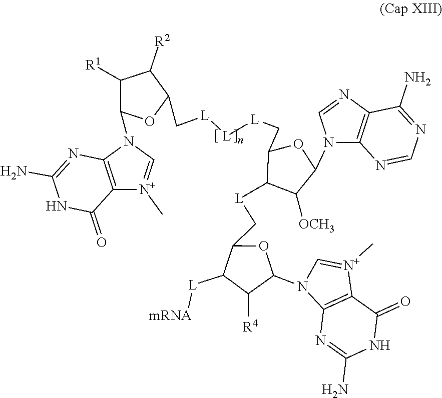

wherein, R¹, R², and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R⁴ is OH. In some embodiments, n is 1.

### Poly-Adenine (Poly-A) Tail

Polyadenylation is the addition of a poly (A) tail, a chain of adenine nucleotides usually about 100-120 monomers in length, to an mRNA. In eukaryotes, polyadenylation is part of the process that produces mature mRNA for translation and begins as the transcription of a gene terminates. The 3′-most segment of a newly made pre-mRNA is first cleaved off by a set of proteins; these proteins then synthesize the poly (A) tail at the 3′ end. The poly (A) tail is important for the nuclear export, translation, and stability of mRNA. The tail is shortened over time, and, when it is short enough, the mRNA is enzymatically degraded. However, in a few cell types, mRNAs with short poly (A) tails are stored for later activation by re-polyadenylation in the cytosol.

Preferably, an mRNA described herein comprises a 3′ tail region, which can serve to protect the mRNA from exonuclease degradation. The tail region may be a 3′poly (A) and/or 3′poly (C) region. Preferably, the tail region is a 3′ poly(A) tail. As used herein a “3′ poly (A) tail” is a polymer of sequential adenine nucleotides that can range in size from, for example: 10 to 250 sequential adenine nucleotides; 60-125 sequential adenine nucleotides, 90-125 sequential adenine nucleotides, 95-125 sequential adenine nucleotides, 95-121 sequential adenine nucleotides, 100 to 121 sequential adenine nucleotides, 110-121 sequential adenine nucleotides; 112-121 sequential adenine nucleotides; 114-121 adenine sequential nucleotides; or 115 to 121 sequential adenine nucleotides. Preferably, a 3′ poly(A) tail as described herein comprise 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125 sequential adenine nucleotides. 3′ Poly (A) tails can be added using a variety of methods known in the art, e.g., using poly (A) polymerase to add tails to synthetic or in vitro transcribed RNA. Other methods include the use of a transcription vector to encode poly (A) tails or the use of a ligase (e.g., via splint ligation using a T4 RNA ligase and/or T4 DNA ligase), wherein poly (A) may be ligated to the 3′ end of a sense RNA. In some embodiments, a combination of any of the above methods is utilized.

Design and Synthesis of mRNA

The constructs for preferred mRNA sequences of the present disclosure are provided in Table 5.

TABLE 5
Exemplary mRNA Constructs

| | | | | | | | mRNA |
|---|---|---|---|---|---|---|---|
| mRNA | | | | | | | Construct |
| Construct | | | | OTC Protein | | 3′ Poly A | SEQ ID |
| No. | Cap | 5′UTR | Kozak* | Encoded | 3′UTR | Tail | NO: |
| 563 | Cap1 | TEV | Yes | SEQ ID NO: 3 | XBG | Yes | 26 |
| 564 | Cap1 | TEV | Yes | SEQ ID NO: 3 | XBG | Yes | 27 |
| 565 | Cap1 | TEV | Yes | SEQ ID NO: 3 | XBG | Yes | 28 |
| 566 | Cap1 | TEV | Yes | SEQ ID NO: 3 | XBG | Yes | 29 |
| 567 | Cap1 | TEV | Yes | SEQ ID NO: 3 | XBG | Yes | 30 |
| 568 | Cap1 | TEV | Yes | SEQ ID NO: 3 | XBG | Yes | 31 |
| 569 | Cap1 | TEV | Yes | SEQ ID NO: 3 | XBG | Yes | 32 |
| 570 | Cap1 | TEV | Yes | SEQ ID NO: 3 | XBG | Yes | 33 |
| 571 | Cap1 | TEV | Yes | SEQ ID NO: 3 | XBG | Yes | 34 |
| 572 | Cap1 | TEV | Yes | SEQ ID NO: 3 | XBG | Yes | 35 |
| 573 | Cap1 | TEV | Yes | SEQ ID NO: 3 | XBG | Yes | 36 |
| 574 | Cap1 | TEV | Yes | SEQ ID NO: 3 | XBG | Yes | 37 |
| 575 | Cap1 | TEV | P | SEQ ID NO: 3 | XBG | Yes | 38 |
| 708 | Cap1 | TEV | P | SEQ ID NO: 3 | XBG | Yes | 39 |
| 709 | Cap1 | TEV | P | SEQ ID NO: 3 | XBG | Yes | 40 |
| 710 | Cap1 | TEV | P | SEQ ID NO: 3 | XBG | Yes | 41 |
| 711 | Cap1 | TEV | P | SEQ ID NO: 3 | XBG | Yes | 42 |
| 712 | Cap1 | TEV | Yes | SEQ ID NO: 3 | XBG | Yes | 43 |
| 713 | Cap1 | TEV | Yes | SEQ ID NO: 3 | XBG | Yes | 44 |
| 714 | Cap1 | TEV | Yes | SEQ ID NO: 3 | XBG | Yes | 45 |
| 715 | Cap1 | TEV | Yes | SEQ ID NO: 3 | XBG | Yes | 46 |
| 716 | Cap1 | TEV | Yes | SEQ ID NO: 3 | XBG | Yes | 47 |
| 717 | Cap1 | TEV | Yes | SEQ ID NO: 3 | XBG | Yes | 48 |
| 718 | Cap1 | TEV | Yes | SEQ ID NO: 3 | XBG | Yes | 49 |
| 719 | Cap1 | TEV | Yes | SEQ ID NO: 3 | XBG | Yes | 50 |
| 720 | Cap1 | TEV | Yes | SEQ ID NO: 3 | XBG | Yes | 51 |
| 721 | Cap1 | TEV | Yes | SEQ ID NO: 3 | XBG | Yes | 52 |
| 722 | Cap1 | TEV | Yes | SEQ ID NO: 3 | XBG | Yes | 53 |
| 723 | Cap1 | TEV | Yes | SEQ ID NO: 3 | XBG | Yes | 54 |
| 724 | Cap1 | TEV | Yes | SEQ ID NO: 3 | XBG | Yes | 55 |
| 725 | Cap1 | TEV | Yes | SEQ ID NO: 3 | XBG | Yes | 56 |
| 726 | Cap1 | TEV | Yes | SEQ ID NO: 3 | XBG | Yes | 57 |
| 727 | Cap1 | TEV | Yes | SEQ ID NO: 3 | XBG | Yes | 58 |
| 728 | Cap1 | TEV | Yes | SEQ ID NO: 3 | XBG | Yes | 59 |
| 729 | Cap1 | TEV | Yes | SEQ ID NO: 3 | XBG | Yes | 60 |
| 1787 | Cap1 | ARC5-2 | No | SEQ ID NO: 3 | Hu haptoglob | Yes | 61 |
| 1788 | Cap1 | AT1G58420 | P | SEQ ID NO: 3 | Hu ApoE | Yes | 62 |
| 1789 | Cap1 | ARC5-2 | No | SEQ ID NO: 3 | Hu haptoglob | Yes | 63 |
| 1790 | Cap1 | AT1G58420 | P | SEQ ID NO: 3 | Hu ApoE | Yes | 64 |
| 1791 | Cap1 | ARC5-2 | No | SEQ ID NO: 3 | Hu haptoglob | Yes | 65 |
| 1792 | Cap1 | HCV5′ | P | SEQ ID NO: 3 | HCV3′ | Yes | 66 |
| 1793 | Cap1 | AT1G58420 | P | SEQ ID NO: 3 | Hu a-glob | Yes | 67 |
| 1794 | Cap1 | AT1G58420 | P | SEQ ID NO: 3 | Hu a-glob | Yes | 68 |
| 1795 | Cap1 | AT1G58420 | P | SEQ ID NO: 3 | Hu a-glob | Yes | 69 |
| 1796 | Cap1 | Hu alb | No | SEQ ID NO: 3 | Ms Alb | Yes | 70 |
| 1797 | Cap1 | Hu alb | No | SEQ ID NO: 3 | Ms Alb | Yes | 71 |
| 1798 | Cap1 | Hu alb | No | SEQ ID NO: 3 | Ms Alb | Yes | 72 |
| 1799 | Cap1 | AT1G58420 | Yes | SEQ ID NO: 3 | Hu a-glob | Yes | 73 |
| 1800 | Cap1 | Hu alb | No | SEQ ID NO: 3 | Ms Alb | Yes | 74 |
| 1801 | Cap1 | AT1G58420 | P | SEQ ID NO: 3 | Hu ApoE | Yes | 75 |
| 1802 | Cap1 | ARC5-2 | No | SEQ ID NO: 3 | Hu haptoglob | Yes | 76 |
| 1803 | Cap1 | TEV | Yes | SEQ ID NO: 3 | XBG | Yes | 77 |
| 1804 | Cap1 | TEV | Yes | SEQ ID NO: 3 | XBG | Yes | 78 |
| 1805 | Cap1 | TEV | Yes | SEQ ID NO: 3 | XBG | Yes | 79 |
| 1806 | Cap1 | TEV | Yes | SEQ ID NO: 3 | XBG | Yes | 80 |
| 1808 | Cap1 | TEV | Yes | SEQ ID NO: 3 | XBG | Yes | 81 |
| 1809 | Cap1 | TEV | Yes | SEQ ID NO: 3 | XBG | Yes | 82 |
| 1816 | Cap1 | TEV | P | SEQ ID NO: 3 | XBG | Yes | 83 |
| 1822 | Cap1 | TEV | Yes | SEQ ID NO: 3 | XBG | Yes | 84 |
| 1823 | Cap1 | TEV | Yes | SEQ ID NO: 3 | XBG | Yes | 85 |
| 1840 | Cap1 | EMCV | No | SEQ ID NO: 3 | EMCV | Yes | 86 |
| 1841 | Cap1 | EMCV | No | SEQ ID NO: 3 | EMCV | Yes | 87 |
| 1842 | Cap1 | EMCV | No | SEQ ID NO: 3 | EMCV | Yes | 88 |
| 1843 | Cap1 | HSP70-P2- | Yes | SEQ ID NO: 3 | XBG | Yes | 89 |
| | | TEV | | | | | |
| 1844 | Cap1 | HSP70-M1- | Yes | SEQ ID NO: 3 | XBG | Yes | 90 |
| | | TEV | | | | | |
| 1845 | Cap1 | HSP70-M2- | Yes | SEQ ID NO: 3 | XBG | Yes | 91 |
| | | TEV | | | | | |
| 1846 | Cap1 | HSP17.9- | Yes | SEQ ID NO: 3 | XBG | Yes | 92 |
| | | TEV | | | | | |
| 1847 | Cap1 | HSP70-P1- | Yes | SEQ ID NO: 3 | XBG | Yes | 93 |
| | | TEV | | | | | |
| 1882 | Cap1 | TEV | Yes | SEQ ID NO: 3 | XBG | Yes | 94 |
| 1883 | Cap1 | TEV | Yes | SEQ ID NO: 3 | XBG | Yes | 95 |
| 1884 | Cap1 | TEV | Yes | SEQ ID NO: 3 | XBG | Yes | 96 |
| 1885 | Cap1 | TEV | Yes | SEQ ID NO: 3 | XBG | Yes | 97 |
| 1886 | Cap1 | TEV | Yes | SEQ ID NO: 3 | XBG | Yes | 98 |
| 1887 | Cap1 | TEV | Yes | SEQ ID NO: 3 | XBG | Yes | 99 |
| 1888 | Cap1 | TEV | Yes | SEQ ID NO: 3 | XBG | Yes | 100 |
| 1889 | Cap1 | TEV | Yes | SEQ ID NO: 3 | XBG | Yes | 101 |
| 1890 | Cap1 | TEV | Yes | SEQ ID NO: 3 | XBG | Yes | 102 |
| 1891 | Cap1 | TEV | Yes | SEQ ID NO: 3 | XBG | Yes | 103 |
| 1898 | Cap1 | TEV | Yes | SEQ ID NO: 3 | XBG | Yes | 104 |
| 1899 | Cap1 | TEV | Yes | SEQ ID NO: 3 | XBG | Yes | 105 |
| 1900 | Cap1 | TEV | Yes | SEQ ID NO: 3 | XBG | Yes | 106 |
| 1903 | Cap1 | TEV | P | SEQ ID NO: 3 | XBG | Yes | 107 |
| 1904 | Cap1 | TEV | P | SEQ ID NO: 3 | XBG | Yes | 108 |
| 1905 | Cap1 | TEV | P | SEQ ID NO: 3 | XBG | Yes | 109 |
| 1906 | Cap1 | TEV | P | SEQ ID NO: 3 | XBG | Yes | 110 |
| 1907 | Cap1 | TEV | P | SEQ ID NO: 3 | XBG | Yes | 111 |
| 1908 | Cap1 | TEV | P | SEQ ID NO: 3 | XBG | Yes | 112 |
| 1915 | Cap1 | HSP70-P1- | Yes | SEQ ID NO: 3 | Hu a-glob | Yes | 113 |
| | | AT1G58420 | | | | | |
| 1916 | Cap1 | HSP70-P1- | Yes | SEQ ID NO: 3 | Hu a-glob | Yes | 114 |
| | | AT1G58420 | | | | | |
| 1917 | Cap1 | HSP70-P1- | Yes | SEQ ID NO: 3 | Hu a-glob | Yes | 115 |
| | | AT1G58420 | | | | | |
| 1918 | Cap1 | HSP70-P1- | Yes | SEQ ID NO: 3 | Hu a-glob | Yes | 116 |
| | | AT1G58420 | | | | | |
| 1919 | Cap1 | AT1G58420 | Yes | SEQ ID NO: 3 | Hu a-glob | Yes | 117 |
| 1920 | Cap1 | AT1G58420 | Yes | SEQ ID NO: 3 | Hu a-glob | Yes | 118 |
| 1921 | Cap1 | AT1G58420 | Yes | SEQ ID NO: 4** | Hu a-glob | Yes | 119 |
| 1925 | Cap1 | TEV | Yes | SEQ ID NO: 3 | XBG | Yes | 120 |
| 1926 | Cap1 | AT1G58420 | Yes | SEQ ID NO: 3 | Hu a-glob | Yes | 121 |
| 1927 | Cap1 | AT1G58420 | Yes | SEQ ID NO: 3 | Hu a-glob | Yes | 122 |
| 1928 | Cap1 | AT1G58420 | Yes | SEQ ID NO: 3 | Hu a-glob | Yes | 123 |
| 1929 | Cap1 | HSP70-P1- | Yes | SEQ ID NO: 3 | Hu a-glob | Yes | 124 |
| | | AT1G58420 | | | | | |
| 2016 | Cap1 | AT1G58420 | Yes | SEQ ID NO: 3 | Hu a-glob | Yes | 253 |
| 2260 | Cap1 | AT1G58420 | Yes | SEQ ID NO: 4** | Hu a-glob | Yes | 251 |
| 2262 | Cap1 | AT1G58420 | Yes | SEQ IS NO: 4** | Hu a-glob | Yes | 252 |

*Kozak sequence defined as GCCACC (SEQ ID NO: 23). Partial (P) Kozak defined as GCCA (SEQ ID NO: 24).
**Construct encodes modified human OTC protein of SEQ ID NO: 4.
***The SEQ ID NOs associated with the constructs of the above table do not show the poly(A) tail as these can vary in length as further described herein.

Preferred mRNA sequences include all of the mRNA sequences listed in Table 5. In some embodiments, mRNA sequences of the present disclosure include all of the mRNA sequences listed in Table 5 in which 0% to 100%, preferably 1% to 100%, preferably 25% to 100%, preferably 50% to 100% and preferably 75% to 100% of the uracil nucleotides of the mRNA sequences are modified. Preferably, 1% to 100% of the uracil nucleotides are N¹-methylpseudouridine or 5-methoxyuridine. Preferably 100% of the uracil nucleotides are N¹-methylpseudouridine. Preferably 100% of the uracil nucleotides are 5-methoxyuridine.

In some embodiments, an mRNA sequence of the present disclosure comprises a 5′ cap, a 5′UTR derived from a gene expressed by Arabidopsis thaliana, an optional translation enhancer sequence, an optional Kozak sequence or partial Kozak sequence, a codon optimized coding sequence (CDS/ORF) coding for an OTC protein, a 3′ UTR and a poly (A) tail. In some embodiments, the codon optimized CDS encodes a protein of SEQ ID NO: 3 or SEQ ID NO: 4. In some embodiments, the 5′ UTR that is derived from a gene expressed by Arabidopsis thaliana is selected from the 5′ UTRs found in Table 2. In some embodiments, the 5′ UTR that is derived from a gene expressed by Arabidopsis thaliana is selected from the group consisting of: SEQ ID NO: 6, SEQ ID NOs: 125-127 and SEQ ID NOs: 230-250. In some embodiments the 5′ UTR sequence is AT1G58420 having at least 90% identity to the sequence of SEQ ID NO: 6. In some embodiments the 5′ UTR sequence is AT1G58420 having the sequence of SEQ ID NO: 6. In some embodiments, the uracil content of the codon optimized sequence has been reduced with respect to the percentages of uracil content of SEQ ID NO: 1. In some embodiments, 0% to 100% of the uracil nucleotides of the mRNA sequences are modified. In some embodiments, 0% to 100% of the uracil nucleotides are N¹-methylpseudouridine or 5-methoxyuridine. In some embodiments 100% of the uracil nucleotides are N¹-methylpseudouridine. In some embodiments 100% of the uracil nucleotides are 5-methoxyuridine.

Preferred mRNA constructs comprise codon optimized coding sequences and a 5′ UTR from a gene expressed by Arabidopsis thaliana and are selected from: SEQ ID NOs: 62, 67, 68, 69, 73, 113-119, and 121-127.

A preferred mRNA construct of the disclosure comprises mRNA construct 1921 (SEQ ID NO: 119) having an optimized ORF encoding the modified human OTC protein of SEQ ID NO: 4 and comprising a 3′ Poly A tail of 121 nucleotides. Another preferred mRNA construct comprises construct 2260 (SEQ ID NO: 251) encoding the modified human OTC protein of SEQ ID NO: 4 and comprising a 3′ Poly (A) tail of 100 nucleotides. Another preferred mRNA construct comprises construct 2262 (SEQ ID NO: 252) encoding the modified human OTC protein of SEQ ID NO: 4 and comprising a 3′ Poly (A) tail of 100 nucleotides.

A preferred mRNA sequence of the disclosure includes the mRNA construct 1799 (SEQ ID NO:73) having a codon optimized ORF encoding wild type human OTC of SEQ ID NO: 3 and having a 3′ Poly (A) tail of 121 nucleotides. Another preferred mRNA construct of the disclosure includes the mRNA construct 2016 (SEQ ID NO: 253) having a codon optimized ORF encoding wild type human OTC of SEQ ID NO: 3 and comprising a 3′ Poly (A) tail of 100 nucleotides.

In some embodiments 100% of the uridine nucleotides of mRNA constructs 1799, 2016, 1921, 2260 and 2262, are N¹-methylpseudouridine. In some embodiments 100% of the uracil nucleotides of mRNA constructs 1799, 2016, 1921, 2260 and 2262, are 5-methoxyuridine.

The mRNA for use in accordance with this disclosure can exhibit increased translation efficiency. As used herein, translation efficiency refers to a measure of the production of a protein or polypeptide by translation of an mRNA in accordance with the disclosure. In some embodiments, an mRNA of the disclosure can exhibit at least 2-fold, 3-fold, 5-fold, or 10-fold increased translation efficiency in vivo as compared to mRNA encoding SEQ ID NO: 3 or SEQ ID NO: 4 that is not codon optimized in accordance with the disclosure and/or that does not comprise the preferred UTRs of the disclosure. In some embodiments an mRNA of the disclosure can provide at least a 2-fold, 3-fold, 5-fold, or 10-fold increased polypeptide or protein level in vivo as compared to mRNA encoding SEQ ID NO: 3 or SEQ ID NO: 4 that is not codon optimized and/or does not comprise the preferred UTRs of the disclosure. In some embodiments, an mRNA of the disclosure can provide increased levels of a polypeptide or protein in vivo as compared to mRNA encoding SEQ ID NO: 3 or SEQ ID NO: 4 that is not codon optimized in accordance with the disclosure and/or that does not comprise the preferred UTRs of the disclosure. For example, the level of a polypeptide or protein can be increased by about 10%, or about 20%, or about 30%, or about 40%, or about 50%, or more.

In some embodiments the mRNA of the disclosure can provide increased functional half-life in the cytoplasm of mammalian cells over mRNA encoding SEQ ID NO: 3 or SEQ ID NO: 4 that is not codon optimized in accordance with the disclosure and/or that does not comprise the preferred UTRs of the disclosure. The inventive translatable molecules can have increased half-life of activity as compared to mRNA encoding SEQ ID NO: 3 or SEQ ID NO: 4 that is not codon optimized in accordance with the disclosure and/or that does not comprise the preferred UTRs of the disclosure.

In some embodiments, the mRNA of the disclosure can reduce cellular innate immune response as compared to mRNA encoding SEQ ID NO: 3 or SEQ ID NO: 4 that is not codon optimized in accordance with the disclosure and/or that does not comprise the preferred UTRs of the disclosure.

In some embodiments, the mRNA of the disclosure can reduce the dose levels required for efficacious therapy as compared to mRNA encoding SEQ ID NO: 3 or SEQ ID NO: 4 that is not codon optimized in accordance with the disclosure and/or that does not comprise the preferred UTRs of the disclosure.

The mRNA agents of the present disclosure may be obtained by any suitable means. Methods for the manufacture of mRNA are known in the art and would be readily apparent to a person of ordinary skill. An mRNA for use in accordance with the present disclosure may be prepared according to any available technique including, but not limited to chemical synthesis, in vitro transcription (IVT) or enzymatic or chemical cleavage of a longer precursor, etc.

In some embodiments, mRNA is produced from a primary complementary DNA (cDNA) construct. The cDNA constructs can be produced on an RNA template by the action of a reverse transcriptase (e.g., RNA-dependent DNA-polymerase). The process of design and synthesis of the primary cDNA constructs described herein generally includes the steps of gene construction, mRNA production (either with or without modifications) and purification. In the IVT method, a target polynucleotide sequence encoding an OTC protein is first selected for incorporation into a vector which will be amplified to produce a cDNA template. Optionally, the target polynucleotide sequence and/or any flanking sequences may be codon optimized. The cDNA template is then used to produce mRNA through in vitro transcription (IVT). After production, the mRNA may undergo purification and clean-up processes. The steps of which are provided in more detail below.

The step of gene construction may include, but is not limited to gene synthesis, vector amplification, plasmid purification, plasmid linearization and clean-up, and cDNA template synthesis and clean-up. Once a human OTC protein (e.g. SEQ ID NO: 3 or SEQ ID NO: 4) is selected for production, a primary construct is designed. Within the primary construct, a first region of linked nucleosides encoding the polypeptide of interest may be constructed using an open reading frame (ORF) of a selected nucleic acid (DNA or RNA) transcript. The ORF may comprise the wild type ORF, an isoform, variant or a fragment thereof. As used herein, an “open reading frame” or “ORF” is meant to refer to a nucleic acid sequence (DNA or RNA) which is capable of encoding a polypeptide of interest. ORFs often begin with the start codon, ATG and end with a nonsense or termination codon or signal.

The cDNA templates may be transcribed to produce an mRNA sequence described herein using an in vitro transcription (IVT) system. The system typically comprises a transcription buffer, nucleotide triphosphates (NTPs), an RNase inhibitor and a polymerase. The NTPs may be selected from, but are not limited to, those described herein including natural and unnatural (modified) NTPs. The polymerase may be selected from, but is not limited to, T7 RNA polymerase, T3 RNA polymerase and mutant polymerases such as, but not limited to, polymerases able to incorporate modified nucleic acids.

The primary cDNA template or transcribed mRNA sequence may also undergo capping and/or tailing reactions. A capping reaction may be performed by methods known in the art to add a 5′ cap to the 5′ end of the primary construct. Methods for capping include, but are not limited to, using a Vaccinia Capping enzyme (New England Biolabs, Ipswich, Mass.) or capping at initiation of in vitro transcription, by for example, including a capping agent as part of the IVT reaction. (Nuc. Acids Symp. (2009) 53:129). A poly (A) tailing reaction may be performed by methods known in the art, such as, but not limited to, 2′ O-methyltransferase and by methods as described herein. If the primary construct generated from cDNA does not include a poly-T, it may be beneficial to perform the poly (A)—tailing reaction before the primary construct is cleaned.

Codon optimized cDNA constructs encoding an ornithine transcarbamylase (OTC) protein are particularly suitable for generating mRNA sequences described herein. For example, such cDNA constructs may be used as the basis to transcribe, in vitro, a polyribonucleotide encoding an ornithine transcarbamylase (OTC) protein. Table 6 provides a listing of exemplary cDNA ORF templates used for in vitro transcription of the mRNA sequences listed in Table 5.

TABLE 6
Exemplary cDNA Templates

| | | Protein encoded |
|---|---|---|
| DNA | | by cDNA |
| Construct | SEQ | template |
| No***: | ID NO: | SEQ ID NO: |
| p563 | 128 | SEQ ID NO: 3* |
| p564 | 129 | SEQ ID NO: 3* |
| p565 | 130 | SEQ ID NO: 3* |
| p566 | 131 | SEQ ID NO: 3* |
| p567 | 132 | SEQ ID NO: 3* |
| p568 | 133 | SEQ ID NO: 3* |
| p569 | 134 | SEQ ID NO: 3* |
| p570 | 135 | SEQ ID NO: 3* |
| p571 | 136 | SEQ ID NO: 3* |
| p572 | 137 | SEQ ID NO: 3* |
| p573 | 138 | SEQ ID NO: 3* |
| p574 | 139 | SEQ ID NO: 3* |
| p575 | 140 | SEQ ID NO: 3* |
| p708 | 141 | SEQ ID NO: 3* |
| p709 | 142 | SEQ ID NO: 3* |
| p710 | 143 | SEQ ID NO: 3* |
| p711 | 144 | SEQ ID NO: 3* |
| p712 | 145 | SEQ ID NO: 3* |
| p713 | 146 | SEQ ID NO: 3* |
| p714 | 147 | SEQ ID NO: 3* |
| p715 | 148 | SEQ ID NO: 3* |
| p716 | 149 | SEQ ID NO: 3* |
| p717 | 150 | SEQ ID NO: 3* |
| p718 | 151 | SEQ ID NO: 3* |
| p719 | 152 | SEQ ID NO: 3* |
| p720 | 153 | SEQ ID NO: 3* |
| p721 | 154 | SEQ ID NO: 3* |
| p722 | 155 | SEQ ID NO: 3* |
| p723 | 156 | SEQ ID NO: 3* |
| p724 | 157 | SEQ ID NO: 3* |
| p725 | 158 | SEQ ID NO: 3* |
| p726 | 159 | SEQ ID NO: 3* |
| p727 | 160 | SEQ ID NO: 3* |
| p728 | 161 | SEQ ID NO: 3* |
| p729 | 162 | SEQ ID NO: 3* |
| p1787 | 163 | SEQ ID NO: 3* |
| p1788 | 164 | SEQ ID NO: 3* |
| p1789 | 165 | SEQ ID NO: 3* |
| p1790 | 166 | SEQ ID NO: 3* |
| p1791 | 167 | SEQ ID NO: 3* |
| p1792 | 168 | SEQ ID NO: 3* |
| p1793 | 169 | SEQ ID NO: 3* |
| p1794 | 170 | SEQ ID NO: 3* |
| p1795 | 171 | SEQ ID NO: 3* |
| p1796 | 172 | SEQ ID NO: 3* |
| p1797 | 173 | SEQ ID NO: 3* |
| p1798 | 174 | SEQ ID NO: 3* |
| p1799 | 175 | SEQ ID NO: 3* |
| p1800 | 176 | SEQ ID NO: 3* |
| p1801 | 177 | SEQ ID NO: 3* |
| p1802 | 178 | SEQ ID NO: 3* |
| p1803 | 179 | SEQ ID NO: 3* |
| p1804 | 180 | SEQ ID NO: 3* |
| p1805 | 181 | SEQ ID NO: 3* |
| p1806 | 182 | SEQ ID NO: 3* |
| p1808 | 183 | SEQ ID NO: 3* |
| p1809 | 184 | SEQ ID NO: 3* |
| p1816 | 185 | SEQ ID NO: 3* |
| p1822 | 186 | SEQ ID NO: 3* |
| p1823 | 187 | SEQ ID NO: 3* |
| p1840 | 188 | SEQ ID NO: 3* |
| p1841 | 189 | SEQ ID NO: 3* |
| p1842 | 190 | SEQ ID NO: 3* |
| p1843 | 191 | SEQ ID NO: 3* |
| p1844 | 192 | SEQ ID NO: 3* |
| p1845 | 193 | SEQ ID NO: 3* |
| p1846 | 194 | SEQ ID NO: 3* |
| p1847 | 195 | SEQ ID NO: 3* |
| p1882 | 196 | SEQ ID NO: 3* |
| p1883 | 197 | SEQ ID NO: 3* |
| p1884 | 198 | SEQ ID NO: 3* |
| p1885 | 199 | SEQ ID NO: 3* |
| p1886 | 200 | SEQ ID NO: 3* |
| p1887 | 201 | SEQ ID NO: 3* |
| p1888 | 202 | SEQ ID NO: 3* |
| p1889 | 203 | SEQ ID NO: 3* |
| p1890 | 204 | SEQ ID NO: 3* |
| p1891 | 205 | SEQ ID NO: 3* |
| p1898 | 206 | SEQ ID NO: 3* |
| p1899 | 207 | SEQ ID NO: 3* |
| p1900 | 208 | SEQ ID NO: 3* |
| p1903 | 209 | SEQ ID NO: 3* |
| p1904 | 210 | SEQ ID NO: 3* |
| p1905 | 211 | SEQ ID NO: 3* |
| p1906 | 212 | SEQ ID NO: 3* |
| p1907 | 213 | SEQ ID NO: 3* |
| p1908 | 214 | SEQ ID NO: 3* |
| p1915 | 215 | SEQ ID NO: 3* |
| p1916 | 216 | SEQ ID NO: 3* |
| p1917 | 217 | SEQ ID NO: 3* |
| p1918 | 218 | SEQ ID NO: 3* |
| p1919 | 219 | SEQ ID NO: 3* |
| p1920 | 220 | SEQ ID NO: 3* |
| p1921 | 221 | SEQ ID NO: 4** |
| p1925 | 222 | SEQ ID NO: 3* |
| p1926 | 223 | SEQ ID NO: 3* |
| p1927 | 224 | SEQ ID NO: 3* |
| p1928 | 225 | SEQ ID NO: 3* |
| p1929 | 226 | SEQ ID NO: 3* |
| p2016 | 227 | SEQ ID NO: 3* |
| p2260 | 228 | SEQ ID NO: 4** |
| p2262 | 229 | SEQ ID NO: 4** |

*SEQ ID NO: 3 is the amino acid sequence for wild type human OTC.
**SEQ ID NO: 4 is the amino acid sequence for modified human OTC.
***The entire plasmid sequence is not included.

Preferred cDNA template sequences include the DNA sequence of SEQ ID NO: 175 (p1779) having an optimized coding sequence encoding wild type human OTC of SEQ ID NO: 3. Preferred cDNA template sequences also include cDNA sequence of SEQ ID NO: 221 (p1921), having an optimized coding sequence encoding a modified OTC protein of SEQ ID NO: 4.

The present disclosure also provides expression vectors comprising a nucleotide sequence encoding an ornithine transcarbamylase (OTC) protein that is preferably operably linked to at least one regulatory sequence. Regulatory sequences are art-recognized and are selected to direct expression of the encoded polypeptide.

Accordingly, the term regulatory sequence includes promoters, enhancers, and other expression control elements. The design of the expression vector may depend on such factors as the choice of the host cell to be transformed and/or the type of protein desired to be expressed.

The present disclosure also provides polynucleotides (e.g. DNA, RNA, cDNA, mRNA, etc.) encoding a human OTC protein that may be operably linked to one or more regulatory nucleotide sequences in an expression construct, such as a vector or plasmid. In certain embodiments, such constructs are DNA constructs. Regulatory nucleotide sequences will generally be appropriate for a host cell used for expression. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the embodiments of the present disclosure. The promoters may be either naturally occurring promoters, or hybrid promoters that combine elements of more than one promoter.

An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and will vary with the host cell used.

The present disclosure also provides a host cell transfected with an mRNA or DNA described herein which encodes an ornithine transcarbamylase (OTC) polypeptide described herein. In some embodiments, the human OTC polypeptide has the sequence of SEQ ID NO: 4. The host cell may be any prokaryotic or eukaryotic cell. For example, an ornithine transcarbamylase (OTC) polypeptide may be expressed in bacterial cells such as E. coli, insect cells (e.g., using a baculovirus expression system), yeast, or mammalian cells. Other suitable host cells are known to those skilled in the art.

The present disclosure also provides a host cell comprising a vector comprising a polynucleotide which encodes an mRNA sequence of any one of SEQ ID NOs: 26-229.

The present disclosure also provides methods of producing a human wild type OTC protein of SEQ ID NO: 3 or a modified human OTC protein SEQ ID NO: 4. In some embodiments, the OTC protein is SEQ ID NO: 4 and is encoded by mRNA of SEQ ID NO 119. For example, a host cell transfected with an expression vector encoding an OTC protein can be cultured under appropriate conditions to allow expression of the polypeptide to occur. The polypeptide may be secreted and isolated from a mixture of cells and medium containing the polypeptides. Alternatively, the polypeptides may be retained in the cytoplasm or in a membrane fraction and the cells harvested, lysed and the protein isolated. A cell culture includes host cells, media and other byproducts. Suitable media for cell culture are well known in the art.

The expressed OTC proteins described herein can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the (OTC polypeptide).

### Lipid-Based Formulations

Therapies based on the intracellular delivery of nucleic acids to target cells face both extracellular and intracellular barriers. Indeed, naked nucleic acid materials cannot be easily systemically administered due to their toxicity, low stability in serum, rapid renal clearance, reduced uptake by target cells, phagocyte uptake and their ability in activating the immune response, all features that preclude their clinical development. When exogenous nucleic acid material (e.g., mRNA) enters the human biological system, it is recognized by the reticuloendothelial system (RES) as foreign pathogens and cleared from blood circulation before having the chance to encounter target cells within or outside the vascular system. It has been reported that the half-life of naked nucleic acid in the blood stream is around several minutes (Kawabata K, Takakura Y, Hashida MPharm Res. 1995 June; 12 (6): 825-30). Chemical modification and a proper delivery method can reduce uptake by the RES and protect nucleic acids from degradation by ubiquitous nucleases, which increase stability and efficacy of nucleic acid-based therapies. In addition, RNAs or DNAs are anionic hydrophilic polymers that are not favorable for uptake by cells, which are also anionic at the surface. The success of nucleic acid-based therapies thus depends largely on the development of vehicles or vectors that can efficiently and effectively deliver genetic material to target cells and obtain sufficient levels of expression in vivo with minimal toxicity.

Moreover, upon internalization into a target cell, nucleic acid delivery vectors are challenged by intracellular barriers, including endosome entrapment, lysosomal degradation, nucleic acid unpacking from vectors, translocation across the nuclear membrane (for DNA), release at the cytoplasm (for RNA), and so on. Successful nucleic acid-based therapy thus depends upon the ability of the vector to deliver the nucleic acids to the target sites inside of the cells in order to obtain sufficient levels of a desired activity such as expression of a gene.

While several gene therapies have been able to successfully utilize a viral delivery vector (e.g., AAV), lipid-based formulations have been increasingly recognized as one of the most promising delivery systems for RNA and other nucleic acid compounds due to their biocompatibility and their ease of large-scale production. One of the most significant advances in lipid-based nucleic acid therapies happened in August 2018 when Patisiran (ALN-TTR02) was the first siRNA therapeutic approved by the Food and Drug Administration (FDA) and by the European Commission (EC). ALN-TTR02 is an siRNA formulation based upon the so-called Stable Nucleic Acid Lipid Particle (SNALP) transfecting technology. Despite the success of Patisiran, the delivery of nucleic acid therapeutics, including mRNA, via lipid formulations is still under ongoing development.

Some art-recognized lipid-formulated delivery vehicles for nucleic acid therapeutics include, according to various embodiments, polymer based carriers, such as polyethyleneimine (PEI), lipid nanoparticles and liposomes, nanoliposomes, ceramide-containing nanoliposomes, multivesicular liposomes, proteoliposomes, both natural and synthetically-derived exosomes, natural, synthetic and semi-synthetic lamellar bodies, nanoparticulates, micelles, and emulsions. These lipid formulations can vary in their structure and composition, and as can be expected in a rapidly evolving field, several different terms have been used in the art to describe a single type of delivery vehicle. At the same time, the terms for lipid formulations have varied as to their intended meaning throughout the scientific literature, and this inconsistent use has caused confusion as to the exact meaning of several terms for lipid formulations. Among the several potential lipid formulations, liposomes, cationic liposomes, and lipid nanoparticles are specifically described in detail and defined herein for the purposes of the present disclosure.

### Liposomes

Conventional liposomes are vesicles that consist of at least one bilayer and an internal aqueous compartment. Bilayer membranes of liposomes are typically formed by amphiphilic molecules, such as lipids of synthetic or natural origin that comprise spatially separated hydrophilic and hydrophobic domains (Lasic, Trends Biotechnol., 16:307-321, 1998). Bilayer membranes of the liposomes can also be formed by amphiphilic polymers and surfactants (e.g., polymerosomes, niosomes, etc.). They generally present as spherical vesicles and can range in size from 20 nm to a few microns. Liposomal formulations can be prepared as a colloidal dispersion or they can be lyophilized to reduce stability risks and to improve the shelf-life for liposome-based drugs. Methods of preparing liposomal compositions are known in the art and would be within the skill of an ordinary artisan.

Liposomes that have only one bilayer are referred to as being unilamellar, and those having more than one bilayer are referred to as multilamellar. The most common types of liposomes are small unilamellar vesicles (SUV), large unilamellar vesicle (LUV), and multilamellar vesicles (MLV). In contrast to liposomes, lysosomes, micelles, and reversed micelles are composed of monolayers of lipids. Generally, a liposome is thought of as having a single interior compartment, however some formulations can be multivesicular liposomes (MVL), which consist of numerous discontinuous internal aqueous compartments separated by several nonconcentric lipid bilayers.

Liposomes have long been perceived as drug delivery vehicles because of their superior biocompatibility, given that liposomes are basically analogs of biological membranes, and can be prepared from both natural and synthetic phospholipids (Int J Nanomedicine. 2014; 9:1833-1843). In their use as drug delivery vehicles, because a liposome has an aqueous solution core surrounded by a hydrophobic membrane, hydrophilic solutes dissolved in the core cannot readily pass through the bilayer, and hydrophobic compounds will associate with the bilayer. Thus, a liposome can be loaded with hydrophobic and/or hydrophilic molecules. When a liposome is used to carry a nucleic acid such as RNA, the nucleic acid will be contained within the liposomal compartment in an aqueous phase.

### Cationic Liposomes

Liposomes can be composed of cationic, anionic, and/or neutral lipids. As an important subclass of liposomes, cationic liposomes are liposomes that are made in whole or part from positively charged lipids, or more specifically a lipid that comprises both a cationic group and a lipophilic portion. In addition to the general characteristics profiled above for liposomes, the positively charged moieties of cationic lipids used in cationic liposomes provide several advantages and some unique structural features. For example, the lipophilic portion of the cationic lipid is hydrophobic and thus will direct itself away from the aqueous interior of the liposome and associate with other nonpolar and hydrophobic species. Conversely, the cationic moiety will associate with aqueous media and more importantly with polar molecules and species with which it can complex in the aqueous interior of the cationic liposome. For these reasons, cationic liposomes are increasingly being researched for use in gene therapy due to their favorability towards negatively charged nucleic acids via electrostatic interactions, resulting in complexes that offer biocompatibility, low toxicity, and the possibility of the large-scale production required for in vivo clinical applications. Cationic lipids suitable for use in cationic liposomes are listed hereinbelow.

### Lipid Nanoparticles

In contrast to liposomes and cationic liposomes, lipid nanoparticles (LNP) have a structure that includes a single monolayer or bilayer of lipids that encapsulates a compound in a solid phase. Thus, unlike liposomes, lipid nanoparticles do not have an aqueous phase or other liquid phase in its interior, but rather the lipids from the bilayer or monolayer shell are directly complexed to the internal compound thereby encapsulating it in a solid core. Lipid nanoparticles are typically spherical vesicles having a relatively uniform dispersion of shape and size. While sources vary on what size qualifies a lipid particle as being a nanoparticle, there is some overlap in agreement that a lipid nanoparticle can have a diameter in the range of from 10 nm to 1000 nm. However, more commonly they are considered to be smaller than 120 nm or even 100 nm.

For lipid nanoparticle nucleic acid delivery systems, the lipid shell is formulated to include an ionizable cationic lipid which can complex to and associate with the negatively charged backbone of the nucleic acid core. Ionizable cationic lipids with apparent pKa values below about 7 have the benefit of providing a cationic lipid for complexing with the nucleic acid's negatively charged backbone and loading into the lipid nanoparticle at pH values below the pKa of the ionizable lipid where it is positively charged. Then, at physiological pH values, the lipid nanoparticle can adopt a relatively neutral exterior allowing for a significant increase in the circulation half-lives of the particles following i.v. administration. In the context of nucleic acid delivery, lipid nanoparticles offer many advantages over other lipid-based nucleic acid delivery systems including high nucleic acid encapsulation efficiency, potent transfection, improved penetration into tissues to deliver therapeutics, and low levels of cytotoxicity and immunogenicity.

Prior to the development of lipid nanoparticle delivery systems for nucleic acids, cationic lipids were widely studied as synthetic materials for delivery of nucleic acid medicines. In these early efforts, after mixing together at physiological pH, nucleic acids were condensed by cationic lipids to form lipid-nucleic acid complexes known as lipoplexes. However, lipoplexes proved to be unstable and characterized by broad size distributions ranging from the submicron scale to a few microns. Lipoplexes, such as the Lipofectamine® reagent, have found considerable utility for in vitro transfection. However, these first-generation lipoplexes have not proven useful in vivo. The large particle size and positive charge (Imparted by the cationic lipid) result in rapid plasma clearance, hemolytic and other toxicities, as well as immune system activation.

### Lipid-mRNA Formulations

An mRNA as disclosed herein or a pharmaceutically acceptable salt thereof can be incorporated into a lipid formulation (i.e., a lipid-based delivery vehicle).

In the context of the present disclosure, a lipid-based delivery vehicle typically serves to transport a desired mRNA to a target cell or tissue. The lipid-based delivery vehicle can be any suitable lipid-based delivery vehicle known in the art. In some embodiments, the lipid-based delivery vehicle is a liposome, a cationic liposome, or a lipid nanoparticle containing an mRNA of the present disclosure. In some embodiments, the lipid-based delivery vehicle comprises a nanoparticle or a bilayer of lipid molecules and an mRNA of the present disclosure. In some embodiments, the lipid bilayer preferably further comprises a neutral lipid or a polymer. In some embodiments, the lipid formulation preferably comprises a liquid medium. In some embodiments, the formulation preferably further encapsulates a nucleic acid. In some embodiments, the lipid formulation preferably further comprises a nucleic acid and a neutral lipid or a polymer. In some embodiments, the lipid formulation preferably encapsulates the nucleic acid.

The description provides lipid formulations comprising one or more therapeutic mRNA molecules encapsulated within the lipid formulation. In some embodiments, the lipid formulation comprises liposomes. In some embodiments, the lipid formulation comprises cationic liposomes. In some embodiments, the lipid formulation comprises lipid nanoparticles.

In some embodiments, the mRNA is fully encapsulated within the lipid portion of the lipid formulation such that the mRNA in the lipid formulation is resistant in aqueous solution to nuclease degradation. In other embodiments, the lipid formulations described herein are substantially non-toxic to mammals such as humans.

The lipid formulations of the disclosure also typically have a total lipid: RNA ratio (mass/mass ratio) of from about 1:1 to about 100:1, from about 1:1 to about 50:1, from about 2:1 to about 45:1, from about 3:1 to about 40:1, from about 5:1 to about 38:1, or from about 10:1 to about 40:1, or from about 15:1 to about 35:1, or from about 20:1 to about 40:1; or from about 25:1 to about 35:1; or from about 27:1 to about 32:1; or from about 28:1 to about 32:1; or from about 29:1 to about 31:1. In some preferred embodiments, the total lipid: RNA ratio (mass/mass ratio) is from about 25:1 to about 35:1. The ratio may be any value or subvalue within the recited ranges, including endpoints.

The lipid formulations of the present disclosure typically have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, or about 150 nm, and are substantially non-toxic. The diameter may be any value or subvalue within the recited ranges, including endpoints. In addition, nucleic acids, when present in the lipid nanoparticles of the present disclosure, are resistant in aqueous solution to degradation with a nuclease.

In preferred embodiments, the lipid formulations comprise an mRNA, a cationic lipid (e.g., one or more cationic lipids or salts thereof described herein), a phospholipid, and a conjugated lipid that inhibits aggregation of the particles (e.g., one or more PEG-lipid conjugates). The lipid formulations can also include cholesterol.

In the nucleic acid-lipid formulations, the mRNA may be fully encapsulated within the lipid portion of the formulation, thereby protecting the nucleic acid from nuclease degradation. In preferred embodiments, a lipid formulation comprising an mRNA is fully encapsulated within the lipid portion of the lipid formulation, thereby protecting the nucleic acid from nuclease degradation. In certain instances, the mRNA in the lipid formulation is not substantially degraded after exposure of the particle to a nuclease at 37° C. for at least 20, 30, 45, or 60 minutes. In certain other instances, the mRNA in the lipid formulation is not substantially degraded after incubation of the formulation in serum at 37° C. for at least 30, 45, or 60 minutes or at least 2, 3, 4, 5, 6, 7, 8, 9, 10, 12, 14, 16, 18, 20, 22, 24, 26, 28, 30, 32, 34, or 36 hours. In other embodiments, the mRNA is complexed with the lipid portion of the formulation. One of the benefits of the formulations of the present disclosure is that the nucleic acid-lipid compositions are substantially non-toxic to mammals such as humans.

In the context of nucleic acids, full encapsulation may be determined by performing a membrane-impermeable fluorescent dye exclusion assay, which uses a dye that has enhanced fluorescence when associated with nucleic acid. Encapsulation is determined by adding the dye to a lipid formulation, measuring the resulting fluorescence, and comparing it to the fluorescence observed upon addition of a small amount of nonionic detergent. Detergent-mediated disruption of the lipid layer releases the encapsulated nucleic acid, allowing it to interact with the membrane-impermeable dye. Nucleic acid encapsulation may be calculated as E=(I₀−I)/I₀, where/and I₀ refers to the fluorescence intensities before and after the addition of detergent.

In other embodiments, the present disclosure provides a nucleic acid-lipid composition comprising a plurality of nucleic acid-liposomes, nucleic acid-cationic liposomes, or nucleic acid-lipid nanoparticles. In some embodiments, the nucleic acid-lipid composition comprises a plurality of mRNA-liposomes. In some embodiments, the nucleic acid-lipid composition comprises a plurality of mRNA-cationic liposomes. In some embodiments, the nucleic acid-lipid composition comprises a plurality of mRNA-lipid nanoparticles.

In some embodiments, the lipid formulations comprise mRNA that is fully encapsulated within the lipid portion of the formulation, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% (or any fraction thereof or range therein) of the particles have the mRNA encapsulated therein. The amount may be any value or subvalue within the recited ranges, including endpoints.

Depending on the intended use of the lipid formulation, the proportions of the components can be varied, and the delivery efficiency of a particular formulation can be measured using assays known in the art.

According to some embodiments, the expressible polynucleotides and heterologous mRNA constructs described herein are lipid formulated. The lipid formulation is preferably selected from, but not limited to, liposomes, cationic liposomes, and lipid nanoparticles. In one preferred embodiment, a lipid formulation is a cationic liposome or a lipid nanoparticle (LNP) comprising:
(a) an mRNA of the present disclosure,
(b) a cationic lipid,
(c) an aggregation reducing agent (such as polyethylene glycol (PEG) lipid or PEG-modified lipid),
(d) optionally a non-cationic lipid (such as a neutral lipid), and
(e) optionally, a sterol.

In one some embodiments, the cationic lipid is an ionizable cationic lipid. In one embodiment, the lipid nanoparticle formulation consists of (i) at least one cationic lipid; (ii) a helper lipid; (iii) a sterol (e.g., cholesterol); and (iv) a PEG-lipid, in a molar ratio of about 40-70% ionizable cationic lipid: about 2-15% helper lipid: about 20-45% sterol; about 0.5-5% PEG-lipid. Exemplary cationic lipids (including ionizable cationic lipids), helper lipids (e.g., neutral lipids), sterols, and ligand-containing lipids (e.g., PEG-lipids) are described hereinbelow.

### Cationic Lipids

The lipid formulation preferably includes a cationic lipid suitable for forming a cationic liposome or lipid nanoparticle. Cationic lipids are widely studied for nucleic acid delivery because they can bind to negatively charged membranes and induce uptake. Generally, cationic lipids are amphiphiles containing a positive hydrophilic head group, two (or more) lipophilic tails, or a steroid portion and a connector between these two domains. Preferably, the cationic lipid carries a net positive charge at about physiological pH. Cationic liposomes have been traditionally the most commonly used non-viral delivery systems for oligonucleotides, including plasmid DNA, antisense oligos, and siRNA/small hairpin RNA-shRNA). Cationic lipids, such as DOTAP, (1,2-dioleoyl-3-trimethylammonium-propane) and DOTMA (N-[1-(2,3-dioleoyloxy) propyl]-N,N,N-trimethyl-ammonium methyl sulfate) can form complexes or lipoplexes with negatively charged nucleic acids by electrostatic interaction, providing high in vitro transfection efficiency.

In the presently disclosed lipid formulations, the cationic lipid may be, for example, N,N-dioleyl-N,N-dimethylammonium chloride (DODAC), N,N-distearyl-N,N-dimethylammonium bromide (DDAB), 1,2-dioleoyltrimethylammoniumpropane chloride (DOTAP) (also known as N-(2,3-dioleoyloxy) propyl)-N,N,N-trimethylammonium chloride and 1,2-Dioleyloxy-3-trimethylaminopropane chloride salt), N-(1-(2,3-dioleyloxy) propyl)-N,N,N-trimethylammonium chloride (DOTMA), N,N-dimethyl-2,3-dioleyloxy) propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (Y-DLenDMA), 1,2-Dilinoleylcarbamoyloxy-3-dimethylaminopropane (DLin-C-DAP), 1,2-Dilinoleyoxy-3-(dimethylamino) acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.C1), 1,2-Dilinoleoyl-3-trimethylaminopropane chloride salt (DLin-TAP.C1), 1,2-Dilinoleyloxy-3-(N-methylpiperazino) propane (DLin-MPZ), or 3-(N,N-Dilinoleylamino)-1,2-propanediol (DLinAP), 3-(N,N-Dioleylamino)-1,2-propanediol (DOAP), 1,2-Dilinoleyloxo-3-(2-N,N-dimethylamino) ethoxypropane (DLin-EG-DMA), 2,2-Dilinoleyl-4-dimethylaminomethyl-[1,3]-dioxolane (DLin-K-DMA) or analogs thereof, (3aR,5s,6aS)—N,N-dimethyl-2,2-di ((9Z,12Z)-octadeca-9,12-dienyl) tetrahydro-3aH-cyclopenta [d][1,3]dioxol-5-amine, (6Z,9Z,28Z,31Z)-heptatriaconta-6,9,28,31-tetraen-19-yl4-(dimethylamino) butanoate (MC3), 1,1′-(2-(4-(2-((2-(bis (2-hydroxydodecyl) amino) ethyl) (2-hydroxydodecyl) amino)ethyl) piperazin-1-yl) ethylazanediyl) didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), (6Z,9Z,28Z,31Z)-heptatriaconta-6,9,28 31-tetraen-19-yl 4-(dimethylamino) butanoate (DLin-M-C3-DMA), 3-((6Z,9Z,28Z,31Z)-heptatriaconta-6,9,28,3 1-tetraen-19-yloxy)-N,N-dimethylpropan-1-amine (MC3 Ether), 4-((6Z,9Z,28Z,31 Z)-heptatriaconta-6,9,28,31-tetraen-19-yloxy)-N,N-dimethylbutan-1-amine (MC4 Ether), or any combination thereof. Other cationic lipids include, but are not limited to, N,N-distearyl-N,N-dimethylammonium bromide (DDAB), 3P-(N—(N′,N′-dimethylaminoethane)-carbamoyl) cholesterol (DC-Choi), N-(1-(2,3-dioleyloxy) propyl)-N-2-(sperminecarboxamido) ethyl)-N,N-dimethylammonium trifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and Lipofectamine (comprising DOSPA and DOPE, available from GIBCO/BRL).

Other suitable cationic lipids are disclosed in International Publication Nos. WO 09/086558, WO 09/127060, WO 10/048536, WO 10/054406, WO 10/088537, WO 10/129709, and WO 2011/153493; U.S. Patent Publication Nos. 2011/0256175, 2012/0128760, and 2012/0027803; U.S. Pat. Nos. 8,158,601; and Love et al., PNAS, 107 (5), 1864-69, 2010, the contents of which are herein incorporated by reference.

Other suitable cationic lipids include those having alternative fatty acid groups and other dialkylamino groups, including those, in which the alkyl substituents are different (e.g., N-ethyl-N-methylamino-, and N-propyl-N-ethylamino-). These lipids are part of a subcategory of cationic lipids referred to as amino lipids. In some embodiments of the lipid formulations described herein, the cationic lipid is an amino lipid. In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C14 to C22 may be used. Other scaffolds can also be used to separate the amino group and the fatty acid or fatty alkyl portion of the amino lipid.

In some embodiments, the lipid formulation comprises the cationic lipid with Formula I according to the patent application PCT/EP2017/064066. In this context, the disclosure of PCT/EP2017/064066 is also incorporated herein by reference.

In some embodiments, amino or cationic lipids of the present disclosure are ionizable and have at least one protonatable or deprotonatable group, such that the lipid is positively charged at a pH at or below physiological pH (e.g., pH 7.4), and neutral at a second pH, preferably at or above physiological pH. Of course, it will be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the disclosure. In certain embodiments, the protonatable lipids have a pKa of the protonatable group in the range of about 4 to about 11. In some embodiments, the ionizable cationic lipid has a pKa of about 5 to about 7. In some embodiments, the pKa of an ionizable cationic lipid is about 6 to about 7.

In some embodiments, the lipid formulation comprises an ionizable cationic lipid of Formula I:

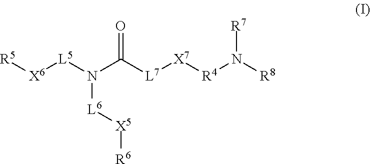

or a pharmaceutically acceptable salt or solvate thereof, wherein R⁵ and R⁶ are each independently selected from the group consisting of a linear or branched C1-C31 alkyl, C2-C31 alkenyl or C₂-C₃₁ alkynyl and cholesteryl; L⁵ and L⁶ are each independently selected from the group consisting of a linear C1-C20 alkyl and C2-C20 alkenyl; X⁵ is-C(O)O—, whereby —C(O)O—R⁶ is formed or —OC(O)-whereby —OC(O)—R⁶ is formed; X⁶ is-C(O)O-whereby —C(O)O—R⁵ is formed or —OC(O)-whereby —OC(O)—R⁵ is formed; X⁷ is S or O; L⁷ is absent or lower alkyl; R⁴ is a linear or branched C₁-C₆ alkyl; and R⁷ and R⁸ are each independently selected from the group consisting of a hydrogen and a linear or branched C₁-C₆ alkyl.

In some embodiments, X⁷ is S.

In some embodiments, X⁵ is-C(O)O—, whereby —C(O)O—R⁶ is formed and X⁶ is —C(O)O-whereby —C(O)O—R⁵ is formed.

In some embodiments, R⁷ and R⁸ are each independently selected from the group consisting of methyl, ethyl and isopropyl.

In some embodiments, L⁵ and L⁶ are each independently a C1-C10 alkyl. In some embodiments, L⁵ is C₁-C₃ alkyl, and L⁶ is C₁-C₈ alkyl. In some embodiments, L⁶ is C₁-C2 alkyl. In some embodiments, L⁵ and L⁶ are each a linear C7 alkyl. In some embodiments, L⁵ and L⁶ are each a linear C9 alkyl.

In some embodiments, R⁵ and R⁶ are each independently an alkenyl. In some embodiments, R⁶ is alkenyl. In some embodiments, R⁶ is C2-C9 alkenyl. In some embodiments, the alkenyl comprises a single double bond. In some embodiments, R⁵ and R⁶ are each alkyl. In some embodiments, R⁵ is a branched alkyl. In some embodiments, R⁵ and R⁶ are each independently selected from the group consisting of a C₉ alkyl, C₉ alkenyl and C₉ alkynyl. In some embodiments, R⁵ and R⁶ are each independently selected from the group consisting of a C11 alkyl, C11 alkenyl and C₁₁ alkynyl. In some embodiments, R⁵ and R⁶ are each independently selected from the group consisting of a C₇alkyl, C₇alkenyl and C₇ alkynyl. In some embodiments, R⁵ is-CH((CH₂)ₚCH₃)₂ or —CH((CH₂)ₚCH₃) ((CH₂)ₚ₋₁CH₃), wherein p is 4-8. In some embodiments, p is 5 and L⁵ is a C₁-C₃ alkyl. In some embodiments, p is 6 and L⁵ is a C₃ alkyl. In some embodiments, p is 7. In some embodiments, p is 8 and L⁵ is a C₁-C₃ alkyl. In some embodiments, R⁵ consists of —CH((CH₂)ₚCH₃) ((CH₂)ₚ₋₁CH₃), wherein p is 7 or 8.

In some embodiments, R⁴ is ethylene or propylene. In some embodiments, R⁴ is n-propylene or isobutylene.

In some embodiments, L⁷ is absent, R⁴ is ethylene, X⁷ is S and R⁷ and R⁸ are each methyl. In some embodiments, L⁷ is absent, R⁴ is n-propylene, X⁷ is S and R⁷ and R⁸ are each methyl. In some embodiments, L⁷ is absent, R⁴ is ethylene, X⁷ is S and R⁷ and R⁸ are each ethyl.

In some embodiments, X⁷ is S, X⁵ is-C(O)O—, whereby —C(O)O—R⁶ is formed, X⁶ is-C(O)O-whereby —C(O)O—R⁵ is formed, L⁵ and L⁶ are each independently a linear C₃-C₇ alkyl, L⁷ is absent, R⁵ is-CH((CH₂)ₚCH₃)₂, and R⁶ is C₇-C₁₂ alkenyl. In some further embodiments, p is 6 and R⁶ is C₉ alkenyl.

In some embodiments, the lipid formulation comprises an ionizable cationic lipid selected from the group consisting of

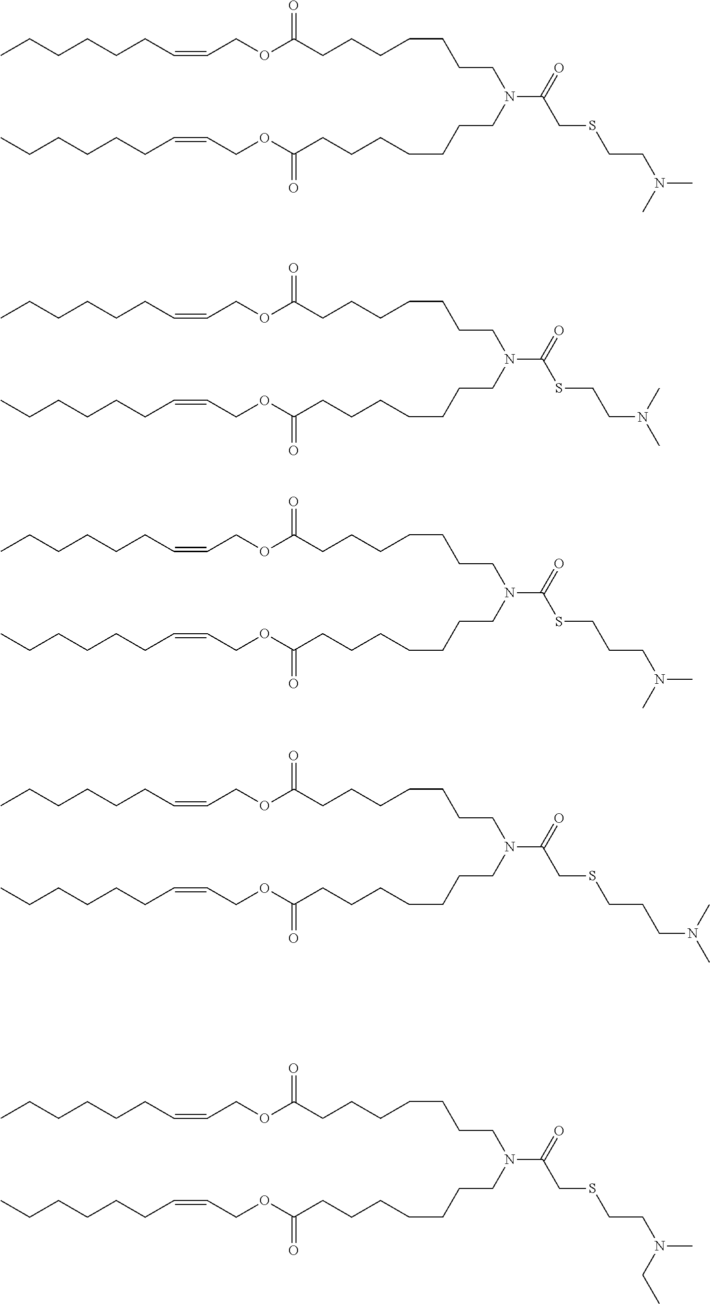

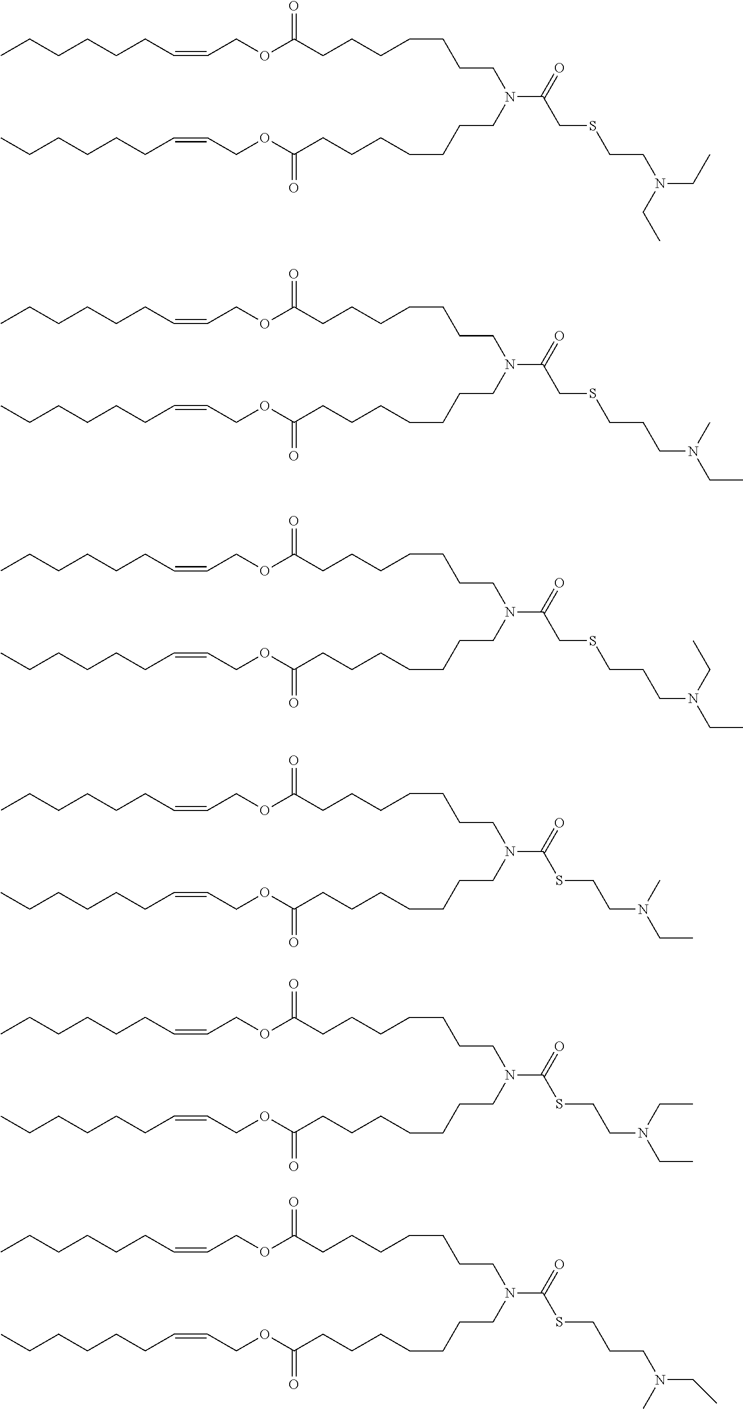

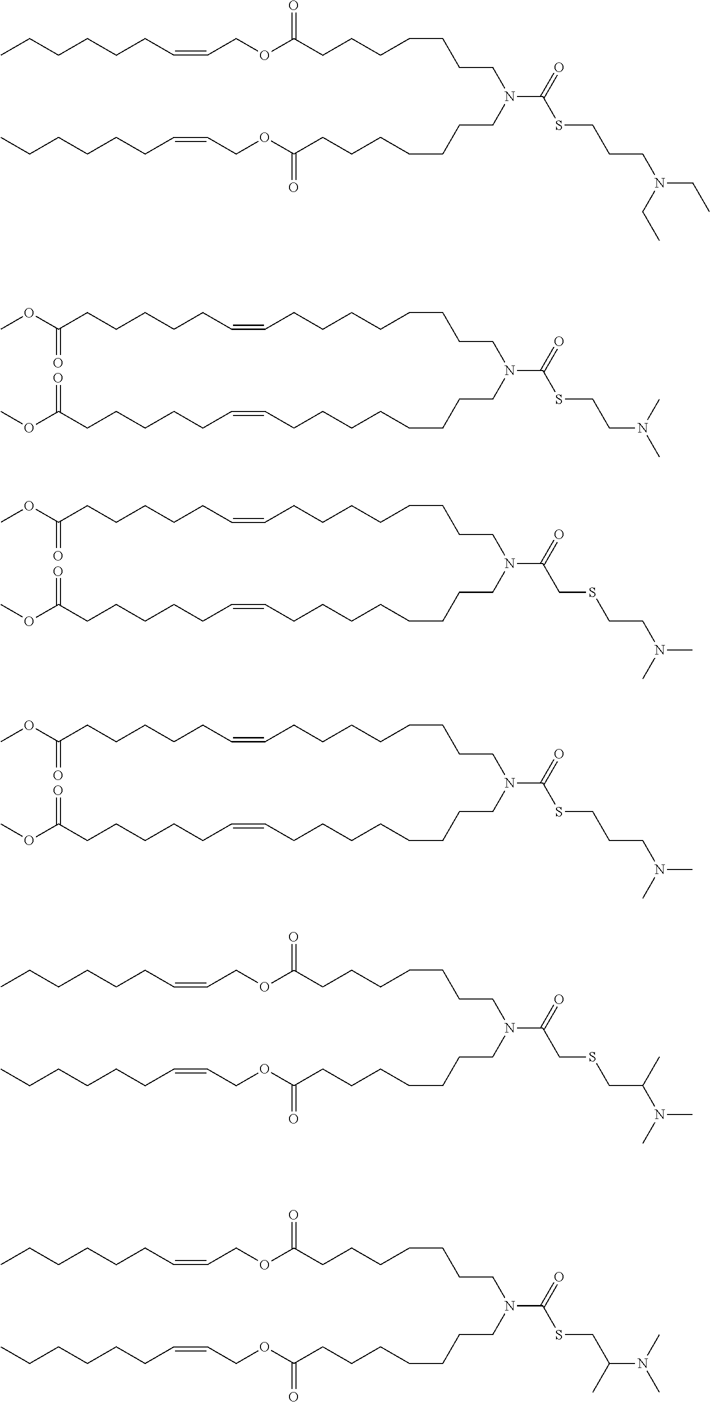

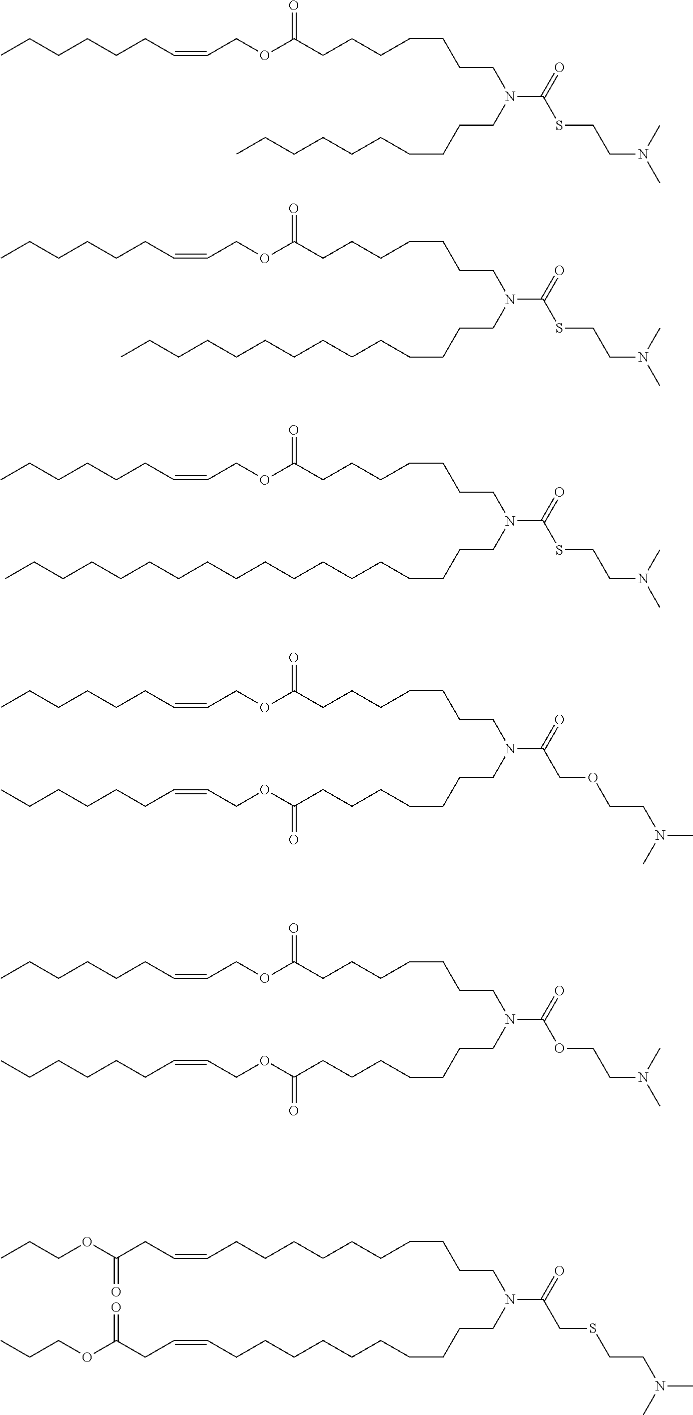

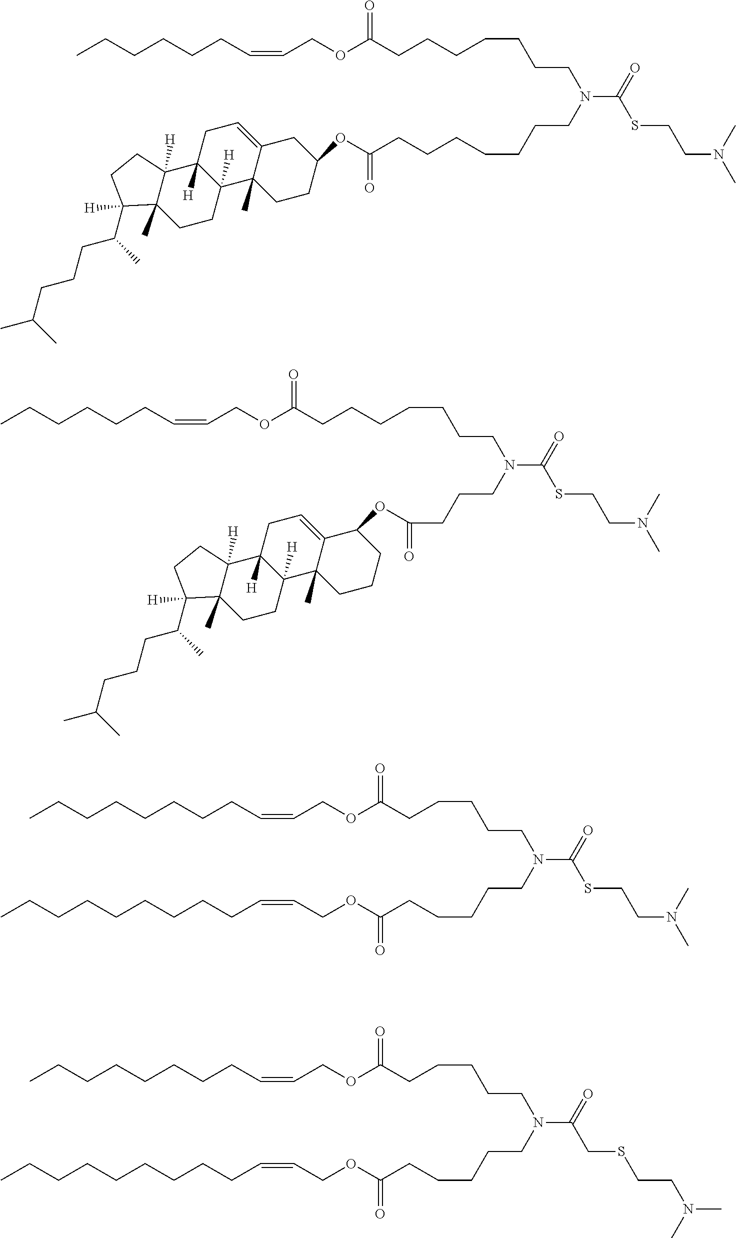

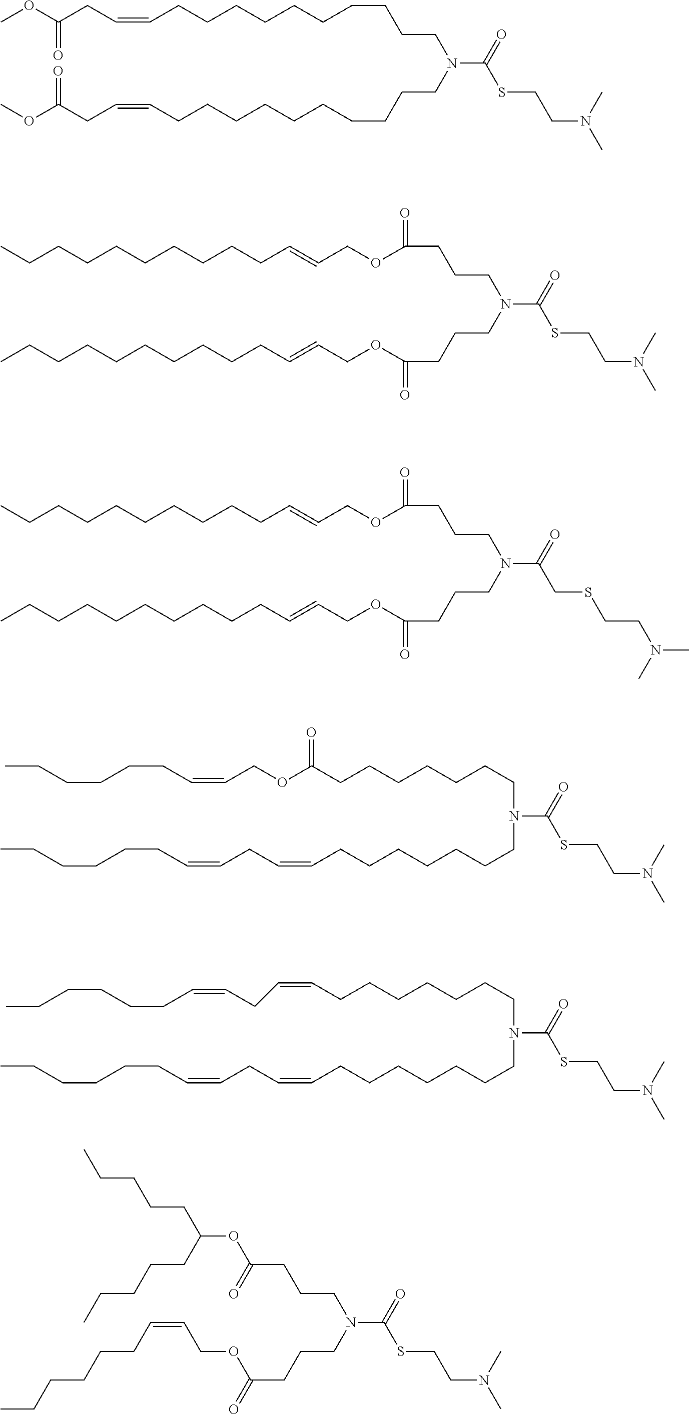

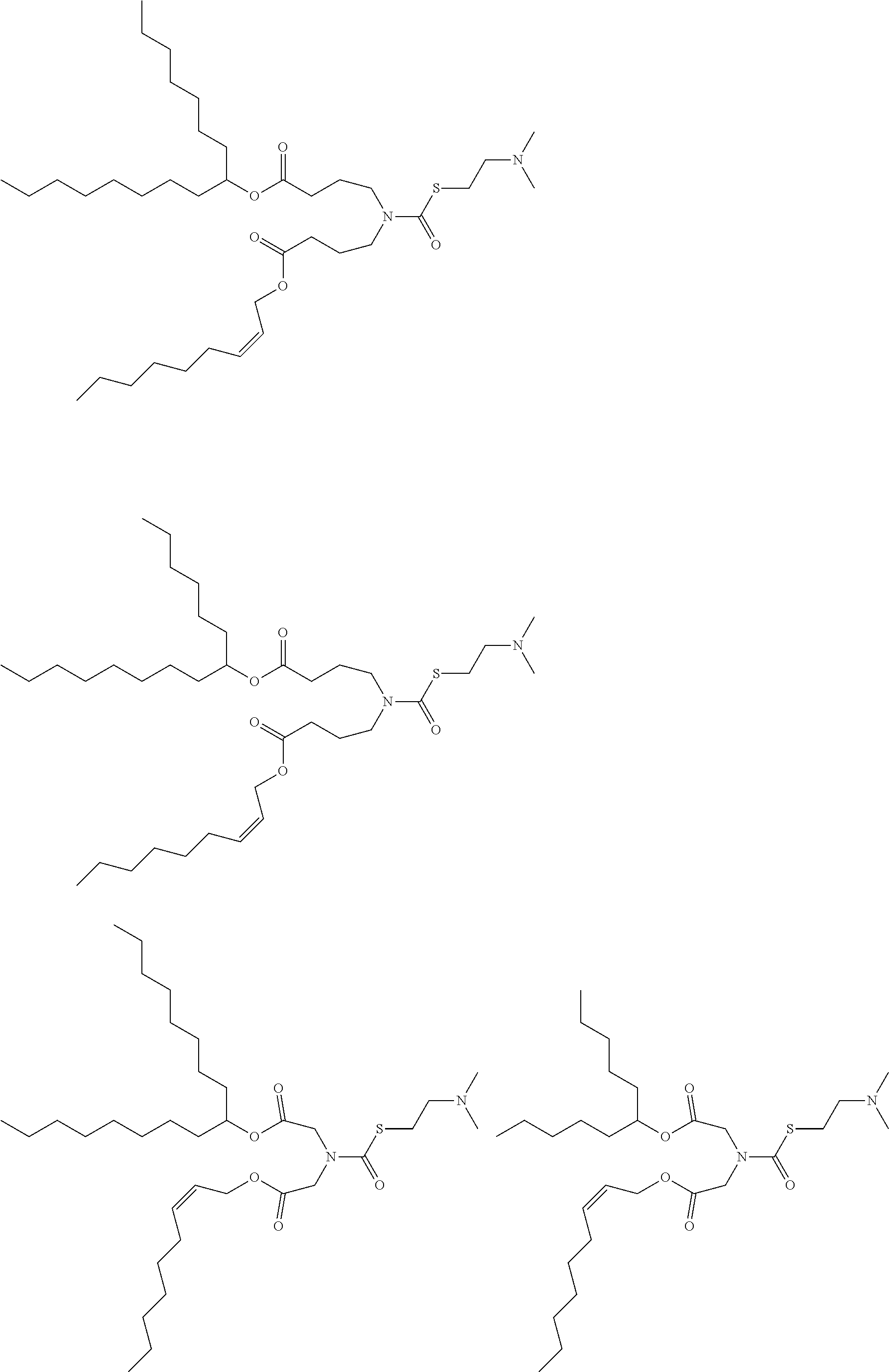

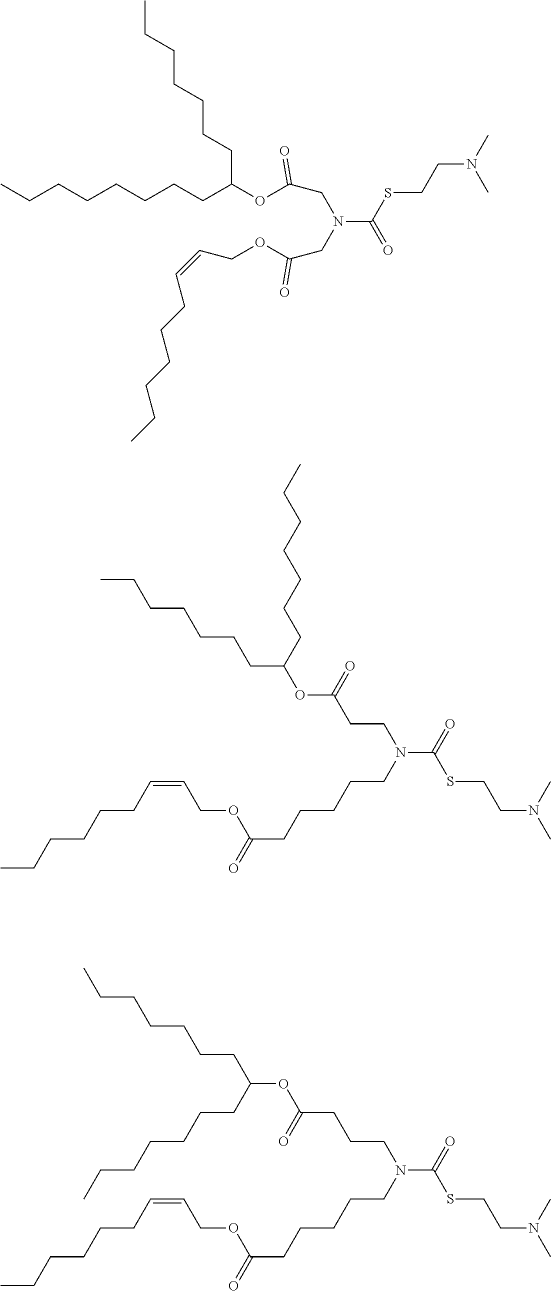

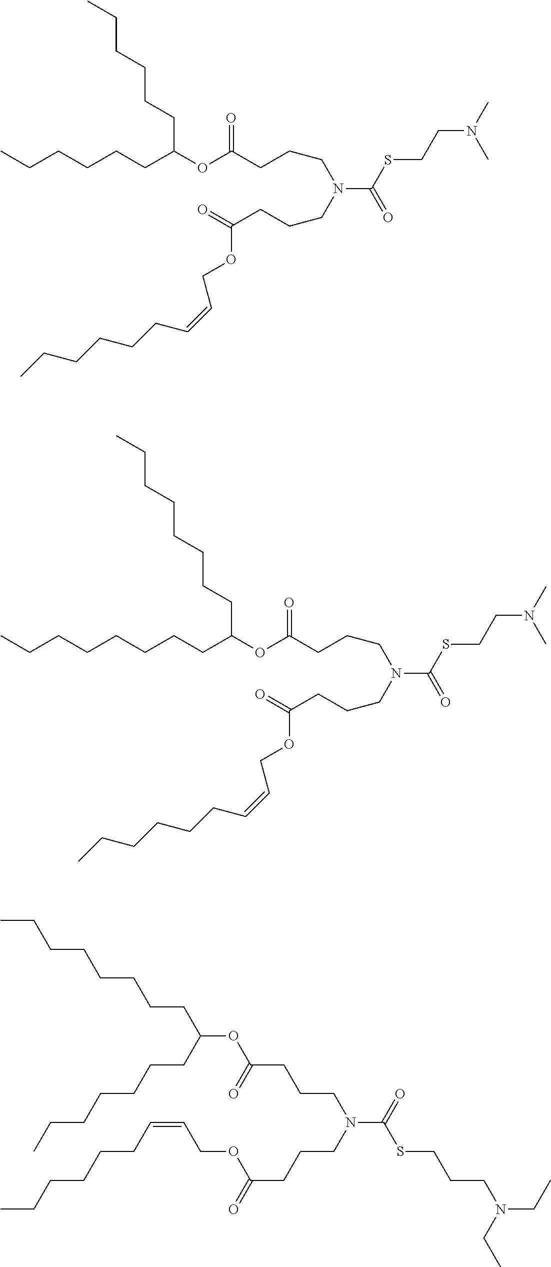

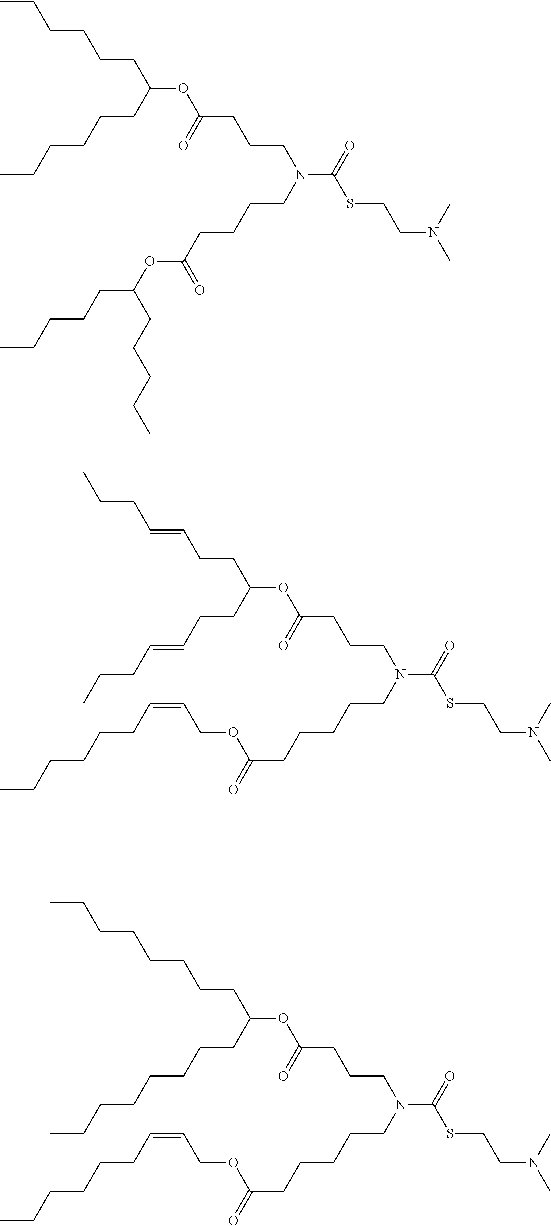

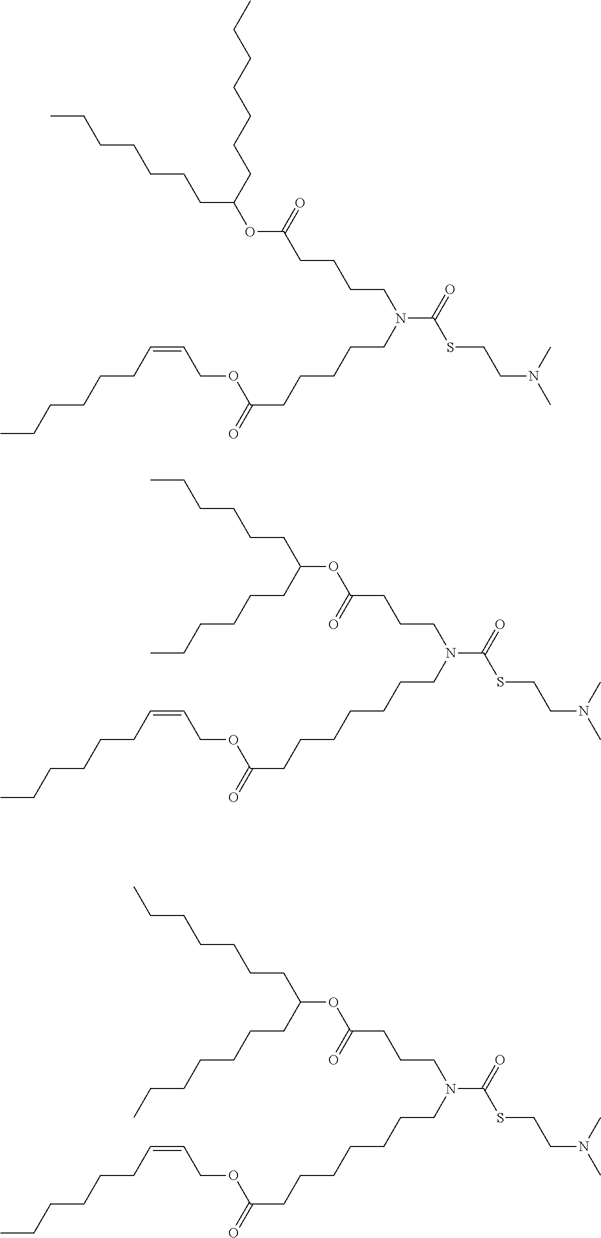

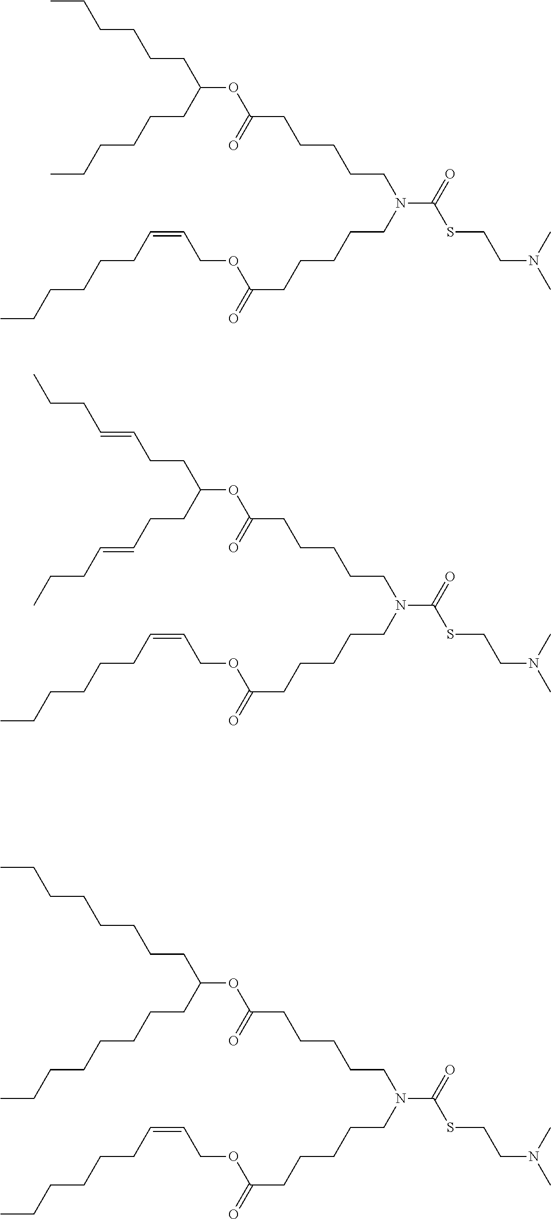

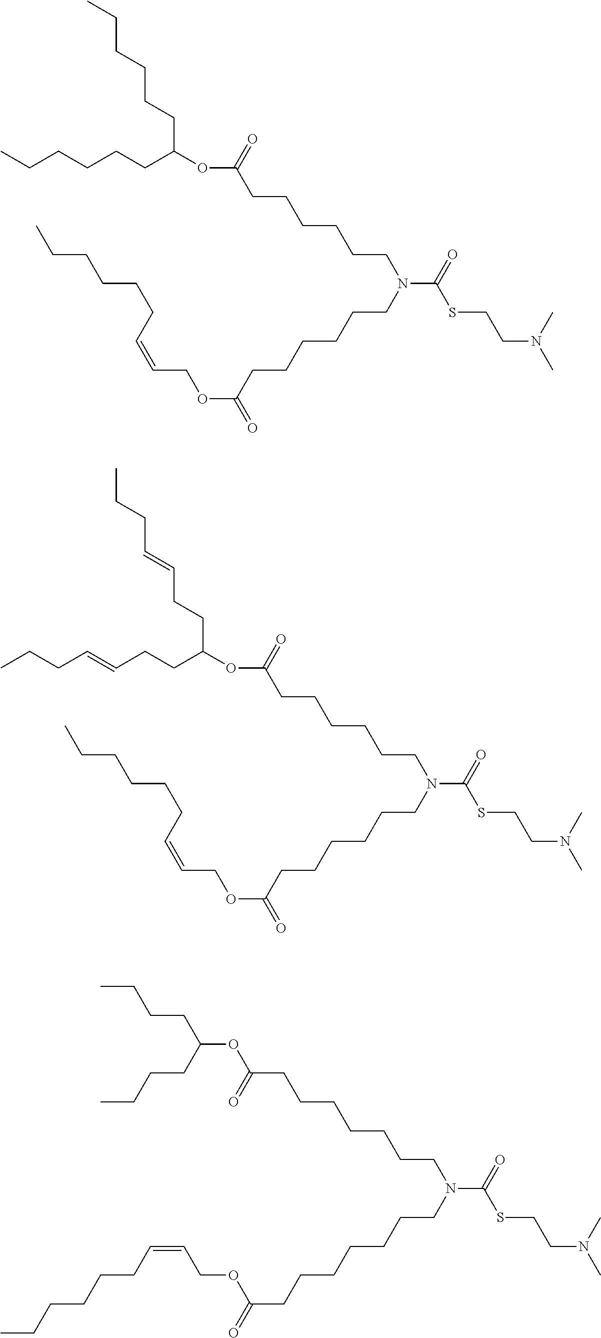

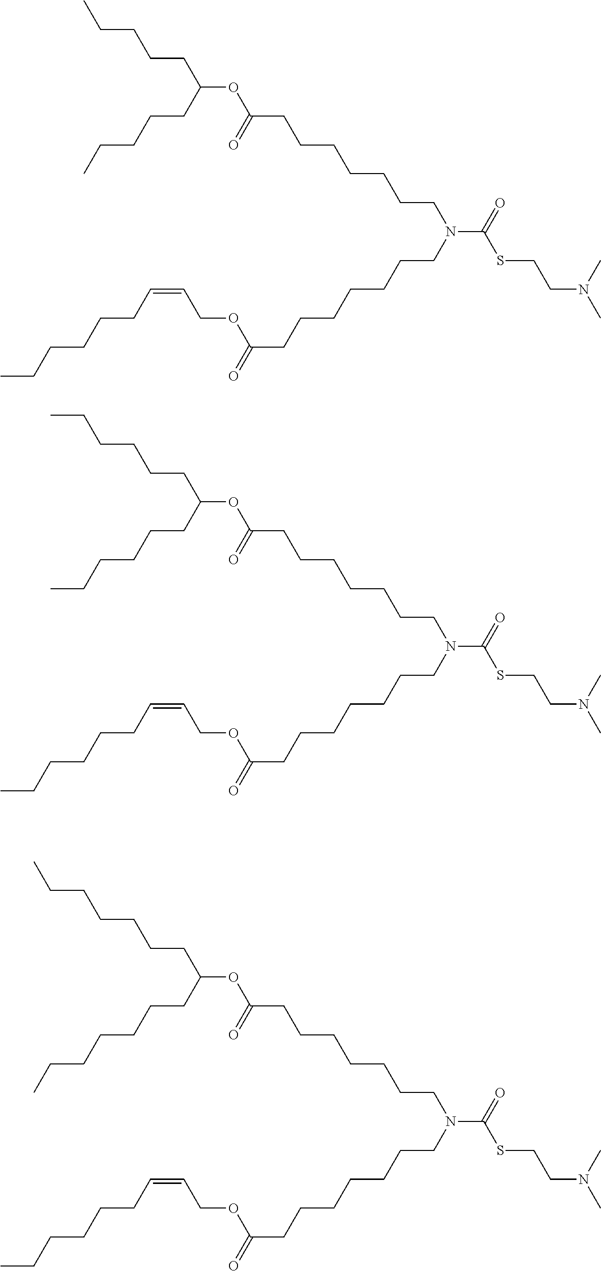

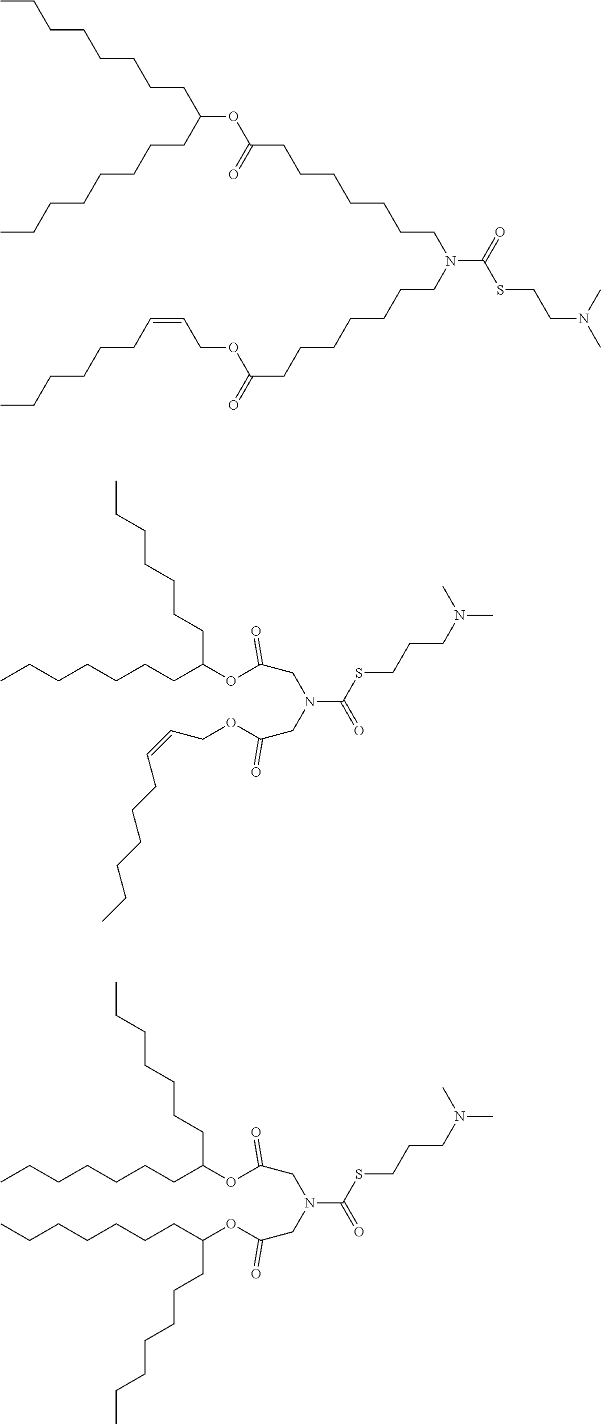

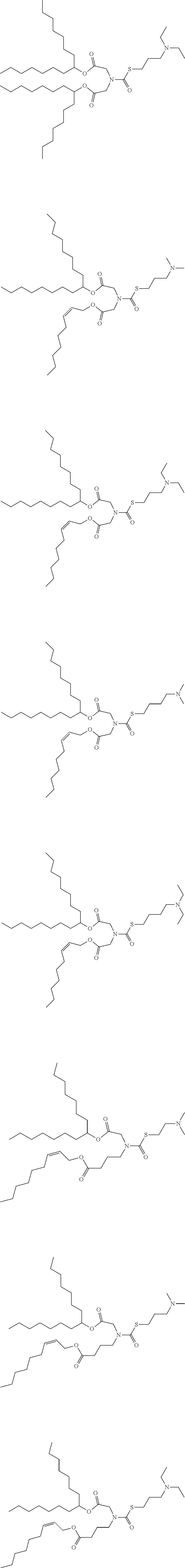

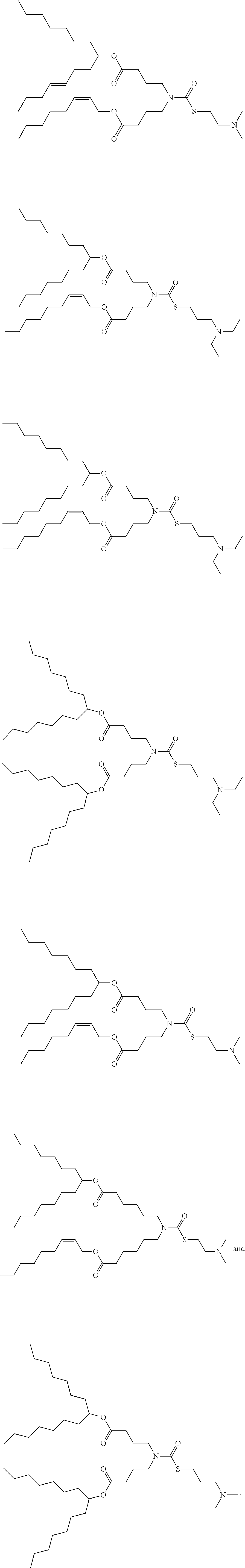

In some embodiments, the lipid formulation can comprise an ionizable cationic lipid selected from the group consisting of LIPID #1 to LIPID #8:

| LIPID # | STRUCTURE |
|---|---|
| 1 | 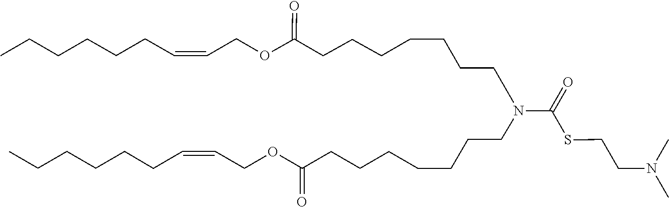 [SMILES: CCCCCC/C=C\COC(=O)CCCCCCCN(CCCCCCCC(=O)OC/C=C\CCCCCC)C(=O)SCCN(C)C] |
| 2 | 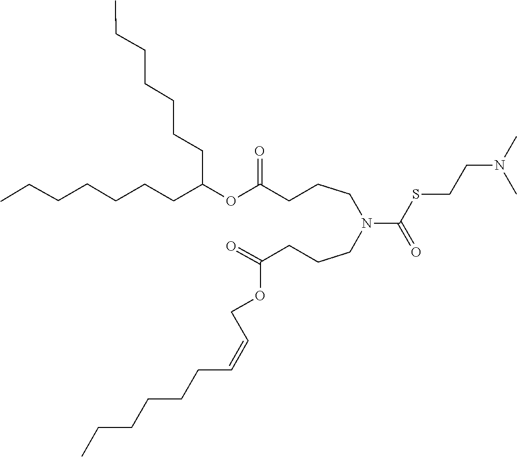 [SMILES: CCCCCC/C=C\COC(=O)CCCN(CCCC(=O)OC(CCCCCCC)CCCCCCC)C(=O)SCCN(C)C] |
| 3 | 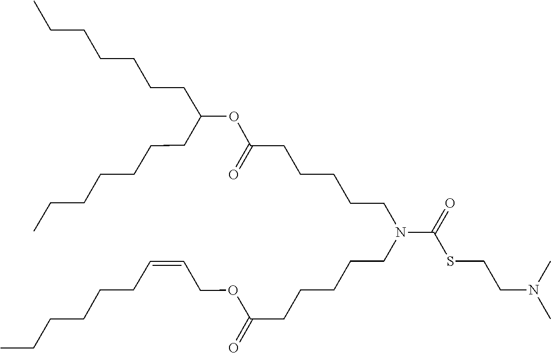 [SMILES: CCCCCC/C=C\COC(=O)CCCCCN(CCCCCC(=O)OC(CCCCCCC)CCCCCCC)C(=O)SCCN(C)C] |
| 4 | 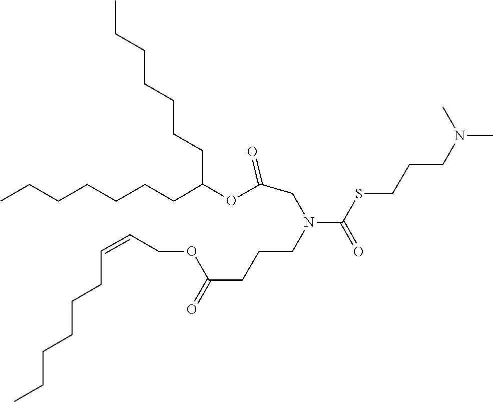 [SMILES: CCCCCC/C=C\COC(=O)CCCN(CC(=O)OC(CCCCCCC)CCCCCCC)C(=O)SCCCN(C)C] |
| 5 | 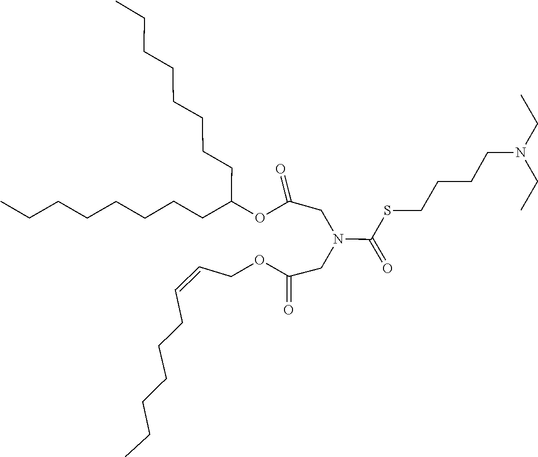 [SMILES: CCCCCC/C=C\COC(=O)CN(CC(=O)OC(CCCCCCCC)CCCCCCCC)C(=O)SCCCCN(CC)CC] |
| 6 | 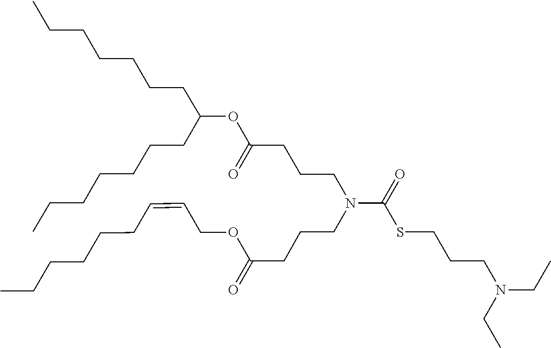 [SMILES: CCCCCC/C=C\COC(=O)CCCN(CCCC(=O)OC(CCCCCCC)CCCCCCC)C(=O)SCCCN(CC)CC] |
| 7 | 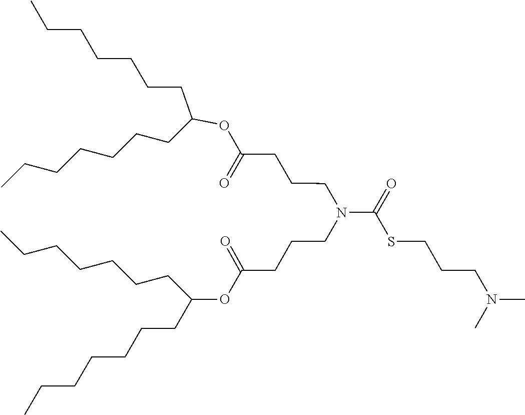 [SMILES: CCCCCCCC(CCCCCCC)OC(=O)CCCN(CCCC(=O)OC(CCCCCCC)CCCCCCC)C(=O)SCCCN(C)C] |
| 8 | 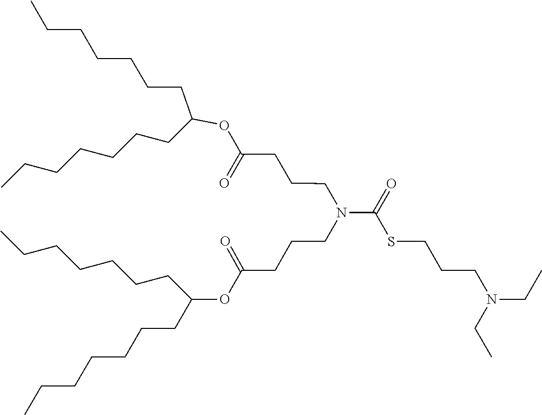 [SMILES: CCCCCCCC(CCCCCCC)OC(=O)CCCN(CCCC(=O)OC(CCCCCCC)CCCCCCC)C(=O)SCCCN(CC)CC] |

In some embodiments, the lipid formulation comprises an ionizable cationic

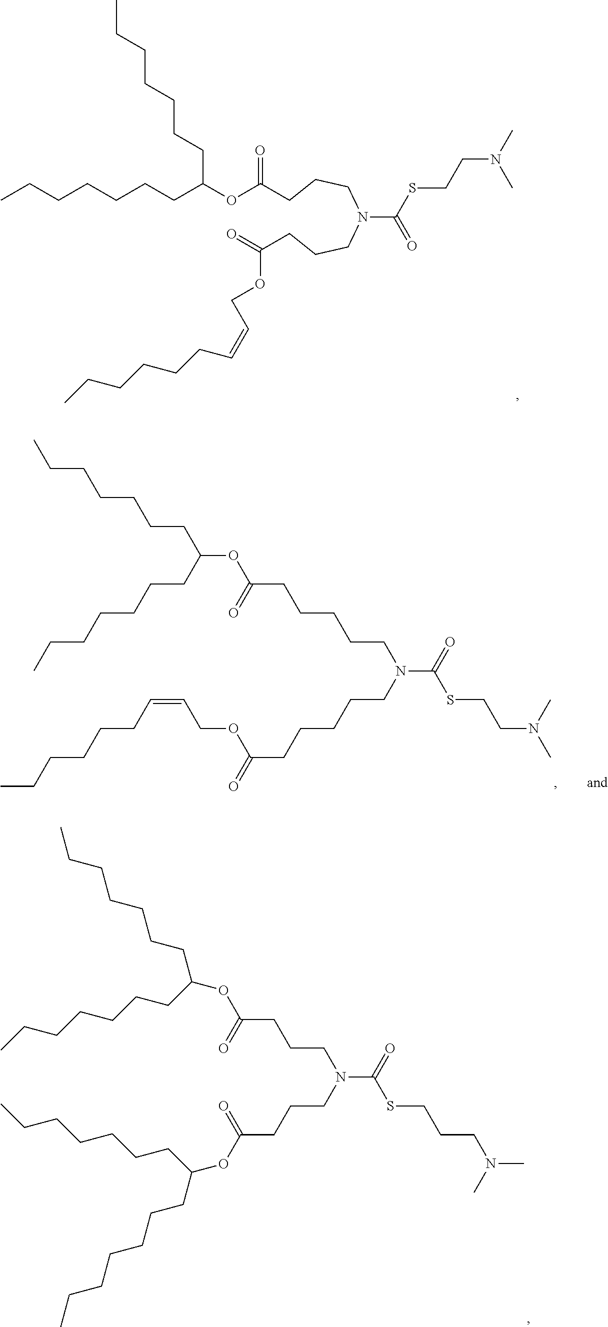

lipid having a structure selected from or a pharmaceutically acceptable salt thereof.

In some preferred embodiments, the lipid formulation comprises an ionizable cationic lipid having the structure

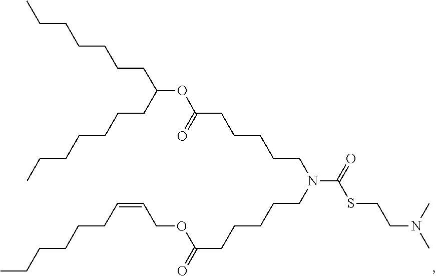 [SMILES: CCCCCC/C=C\COC(=O)CCCCCN(CCCCCC(=O)OC(CCCCCCC)CCCCCCC)C(=O)SCCN(C)C]

or a pharmaceutically acceptable salt thereof.

In embodiments, any one or more lipids recited herein may be expressly excluded.

### Helper Lipids and Sterols

The mRNA-lipid formulations of the present disclosure can comprise a helper lipid, which can be referred to as a neutral helper lipid, non-cationic lipid, non-cationic helper lipid, anionic lipid, anionic helper lipid, or a neutral lipid. It has been found that lipid formulations, particularly cationic liposomes and lipid nanoparticles have increased cellular uptake if helper lipids are present in the formulation. (Curr. Drug Metab. 2014; 15 (9): 882-92). For example, some studies have indicated that neutral and zwitterionic lipids such as 1,2-dioleoylsn-glycero-3-phosphatidylcholine (DOPC), Di-Oleoyl-Phosphatidyl-Ethanoalamine (DOPE) and 1,2-DiStearoyl-sn-glycero-3-PhosphoCholine (DSPC), being more fusogenic (i.e., facilitating fusion) than cationic lipids, can affect the polymorphic features of lipid-nucleic acid complexes, promoting the transition from a lamellar to a hexagonal phase, and thus inducing fusion and a disruption of the cellular membrane. (Nanomedicine (Lond). 2014 January; 9 (1): 105-20). In addition, the use of helper lipids can help to reduce any potential detrimental effects from using many prevalent cationic lipids such as toxicity and immunogenicity.

Non-limiting examples of non-cationic lipids suitable for lipid formulations of the present disclosure include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, palmitoyl, stearoyl, or oleoyl.

Additional examples of non-cationic lipids include sterols such as cholesterol and derivatives thereof. One study concluded that as a helper lipid, cholesterol increases the spacing of the charges of the lipid layer interfacing with the nucleic acid making the charge distribution match that of the nucleic acid more closely. (J. R. Soc. Interface. 2012 Mar. 7; 9 (68): 548-561). Non-limiting examples of cholesterol derivatives include polar analogues such as 5α-cholestanol, 5α-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5α-cholestanone, and cholesteryl decanoate; and mixtures thereof. In preferred embodiments, the cholesterol derivative is a polar analogue such as cholesteryl-(4′-hydroxy)-butyl ether.

In some embodiments, the helper lipid present in the lipid formulation comprises or consists of a mixture of one or more phospholipids and cholesterol or a derivative thereof. In other embodiments, the neutral lipid present in the lipid formulation comprises or consists of one or more phospholipids, e.g., a cholesterol-free lipid formulation. In yet other embodiments, the neutral lipid present in the lipid formulation comprises or consists of cholesterol or a derivative thereof, e.g., a phospholipid-free lipid formulation.

Other examples of helper lipids include nonphosphorous containing lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, and sphingomyelin.

In some embodiments, the helper lipid comprises from about 2 mol % to about 20 mol %, from about 3 mol % to about 18 mol %, from about 4 mol % to about 16 mol %, about 5 mol % to about 14 mol %, from about 6 mol % to about 12 mol %, from about 5 mol % to about 10 mol %, from about 5 mol % to about 9 mol %, or about 2 mol %, about 3 mol %, about 4 mol %, about 5 mol %, about 6 mol %, about 7 mol %, about 8 mol %, about 9 mol %, about 10 mol %, about 11 mol %, or about 12 mol % (or any fraction thereof or the range therein) of the total lipid present in the lipid formulation.

The cholesterol or cholesterol derivative in the lipid formulation may comprise up to about 40 mol %, about 45 mol %, about 50 mol %, about 55 mol %, or about 60 mol % of the total lipid present in the lipid formulation. In some embodiments, the cholesterol or cholesterol derivative comprises about 15 mol % to about 45 mol %, about 20 mol % to about 40 mol %, about 25 mol % to about 35 mol %, or about 28 mol % to about 35 mol %; or about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 36 mol %, or about 37 mol % of the total lipid present in the lipid formulation.

In some embodiments, the phospholipid component in the mixture may comprise from about 2 mol % to about 20 mol %, from about 3 mol % to about 18 mol %, from about 4 mol % to about 16 mol %, about 5 mol % to about 14 mol %, from about 6 mol % to about 12 mol %, from about 5 mol % to about 10 mol %, from about 5 mol % to about 9 mol %, or about 2 mol %, about 3 mol %, about 4 mol %, about 5 mol %, about 6 mol %, about 7 mol %, about 8 mol %, about 9 mol %, about 10 mol %, about 11 mol %, or about 12 mol % (or any fraction thereof or the range therein) of the total lipid present in the lipid formulation.

The percentage of helper lipid present in the lipid formulation is a target amount, and the actual amount of helper lipid present in the formulation may vary, for example, by ±5 mol %.

A lipid formulation containing a cationic lipid compound or ionizable cationic lipid compound may be on a molar basis about 30-70% cationic lipid compound, about 25-40% cholesterol, about 2-15% helper lipid, and about 0.5-5% of a polyethylene glycol (PEG) lipid, wherein the percent is of the total lipid present in the formulation. In some embodiments, the composition is about 40-65% cationic lipid compound, about 25-35% cholesterol, about 3-9% helper lipid, and about 0.5-3% of a PEG-lipid, wherein the percent is of the total lipid present in the formulation.

The formulation may be a lipid particle formulation, for example containing 8-30% nucleic acid compound, 5-30% helper lipid, and 0-20% cholesterol; 4-25% cationic lipid, 4-25% helper lipid, 2-25% cholesterol, 10-35% cholesterol-PEG, and 5% cholesterol-amine; or 2-30% cationic lipid, 2-30% helper lipid, 1-15% cholesterol, 2-35% cholesterol-PEG, and 1-20% cholesterol-amine; or up to 90% cationic lipid and 2-10% helper lipids, or even 100% cationic lipid.

### Lipid Conjugates

The lipid formulations described herein may further comprise a lipid conjugate. The conjugated lipid is useful in that it prevents the aggregation of particles. Suitable conjugated lipids include, but are not limited to, PEG-lipid conjugates, cationic-polymer-lipid conjugates, and mixtures thereof. Furthermore, lipid delivery vehicles can be used for specific targeting by attaching ligands (e.g., antibodies, peptides, and carbohydrates) to its surface or to the terminal end of the attached PEG chains (Front Pharmacol. 2015 Dec. 1; 6:286).

In a preferred embodiment, the lipid conjugate is a PEG-lipid. The inclusion of polyethylene glycol (PEG) in a lipid formulation as a coating or surface ligand, a technique referred to as PEGylation, helps to protects nanoparticles from the immune system and their escape from RES uptake (Nanomedicine (Lond). 2011 June; 6 (4): 715-28). PEGylation has been widely used to stabilize lipid formulations and their payloads through physical, chemical, and biological mechanisms. Detergent-like PEG lipids (e.g., PEG-DSPE) can enter the lipid formulation to form a hydrated layer and steric barrier on the surface. Based on the degree of PEGylation, the surface layer can be generally divided into two types, brush-like and mushroom-like layers. For PEG-DSPE-stabilized formulations, PEG will take on the mushroom conformation at a low degree of PEGylation (usually less than 5 mol %) and will shift to brush conformation as the content of PEG-DSPE is increased past a certain level (Journal of Nanomaterials. 2011; 2011:12). It has been shown that increased PEGylation leads to a significant increase in the circulation half-life of lipid formulations (Annu. Rev. Biomed. Eng. 2011 Aug. 15; 13 ( )507-30; J. Control Release. 2010 Aug. 3; 145 (3): 178-81).

Suitable examples of PEG-lipids include, but are not limited to, PEG coupled to dialkyloxypropyls (PEG-DAA), PEG coupled to diacylglycerol (PEG-DAG), PEG coupled to phospholipids such as phosphatidylethanolamine (PEG-PE), PEG conjugated to ceramides, PEG conjugated to cholesterol or a derivative thereof, and mixtures thereof.

PEG is a linear, water-soluble polymer of ethylene PEG repeating units with two terminal hydroxyl groups. PEGs are classified by their molecular weights and include the following: monomethoxypolyethylene glycol (MePEG-OH), monomethoxypolyethylene glycol-succinate (MePEG-S), monomethoxypolyethylene glycol-succinimidyl succinate (MePEG-S—NHS), monomethoxypolyethylene glycol-amine (MePEG-NH₂), monomethoxypolyethylene glycol-tresylate (MePEG-TRES), monomethoxypolyethylene glycol-imidazolyl-carbonyl (MePEG-IM), as well as such compounds containing a terminal hydroxyl group instead of a terminal methoxy group (e.g., HO-PEG-S, HO-PEG-S—NHS, HO-PEG-NH₂).

The PEG moiety of the PEG-lipid conjugates described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons). In preferred embodiments, the PEG moiety has an average molecular weight of about 2,000 daltons or about 750 daltons. The average molecular weight may be any value or subvalue within the recited ranges, including endpoints.

In certain instances, the PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester-containing linker moieties and ester-containing linker moieties. In a preferred embodiment, the linker moiety is a non-ester-containing linker moiety. Suitable non-ester-containing linker moieties include, but are not limited to, amido (—C(O) NH—), amino (—NR—), carbonyl (—C(O)—), carbamate (—NHC(O)O—), urea (—NHC (O) NH—), disulfide (—S—S—), ether (—O—), succinyl (—(O) CCH2CH₂C(O)—), succinamidyl (—NHC (O) CH₂CH₂C(O) NH—), ether, as well as combinations thereof (such as a linker containing both a carbamate linker moiety and an amido linker moiety). In a preferred embodiment, a carbamate linker is used to couple the PEG to the lipid.

In other embodiments, an ester-containing linker moiety is used to couple the PEG to the lipid. Suitable ester-containing linker moieties include, e.g., carbonate (—OC(O)O—), succinoyl, phosphate esters (—O—(O) POH—O—), sulfonate esters, and combinations thereof.

Phosphatidylethanolamines having a variety of acyl chain groups of varying chain lengths and degrees of saturation can be conjugated to PEG to form the lipid conjugate. Such phosphatidylethanolamines are commercially available or can be isolated or synthesized using conventional techniques known to those of skill in the art. Phosphatidylethanolamines containing saturated or unsaturated fatty acids with carbon chain lengths in the range of C10 to C20 are preferred. Phosphatidylethanolamines with mono- or di-unsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoyl-phosphatidylethanolamine (DOPE), and distearoyl-phosphatidylethanolamine (DSPE).

In some embodiments, the PEG-DAA conjugate is a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, or a PEG-distearyloxypropyl (C₁₈) conjugate. In these embodiments, the PEG preferably has an average molecular weight of about 750 or about 2,000 daltons. In particular embodiments, the terminal hydroxyl group of the PEG is substituted with a methyl group.

In addition to the foregoing, other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl, methacrylamide, polymethacrylamide, and polydimethylacrylamide, polylactic acid, polyglycolic acid, and derivatized celluloses such as hydroxymethylcellulose or hydroxyethylcellulose.

In some embodiments, the lipid conjugate (e.g., PEG-lipid) comprises from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 0.9 mol % to about 1.6 mol %, from about 0.9 mol % to about 1.8 mol %, from about 1 mol % to about 1.8 mol %, from about 1 mol % to about 1.7 mol %, from about 1.2 mol % to about 1.8 mol %, from about 1.2 mol % to about 1.7 mol %, from about 1.3 mol % to about 1.6 mol %, or from about 1.4 mol % to about 1.6 mol % (or any fraction thereof or range therein) of the total lipid present in the lipid formulation. In other embodiments, the lipid conjugate (e.g., PEG-lipid) comprises about 0.5%, 0.6%, 0.7%, 0.8%, 0.9%, 1.0%, 1.2%, 1.3%, 1.4%, 1.5%, 1.6%, 1.7%, 1.8%, 1.9%, 2.0%, 2.5%, 3.0%, 3.5%, 4.0%, 4.5%, or 5%, (or any fraction thereof or range therein) of the total lipid present in the lipid formulation. The amount may be any value or subvalue within the recited ranges, including endpoints.

The percentage of lipid conjugate (e.g., PEG-lipid) present in the lipid formulations of the disclosure is a target amount, and the actual amount of lipid conjugate present in the formulation may vary, for example, by +0.5 mol %. One of ordinary skill in the art will appreciate that the concentration of the lipid conjugate can be varied depending on the lipid conjugate employed and the rate at which the lipid formulation is to become fusogenic.

### Mechanism of Action for Cellular Uptake of Lipid Formulations

Lipid formulations for the intracellular delivery of nucleic acids, particularly liposomes, cationic liposomes, and lipid nanoparticles, are designed for cellular uptake by penetrating target cells through exploitation of the target cells' endocytic mechanisms where the contents of the lipid delivery vehicle are delivered to the cytosol of the target cell. (Nucleic Acid Therapeutics, 28 (3): 146-157, 2018). Specifically, in the case of an ornithine transcarbamylase mRNA-lipid formulation described herein, the mRNA-lipid formulation enters hepatocytes in the liver through Apo-E receptor mediated endocytosis. Prior to endocytosis, functionalized ligands such as PEG-lipid at the surface of the lipid delivery vehicle are shed from the surface, which triggers internalization into the target cell. During endocytosis, some part of the plasma membrane of the cell surrounds the vector and engulfs it into a vesicle that then pinches off from the cell membrane, enters the cytosol and ultimately undergoes the endolysosomal pathway. For ionizable cationic lipid-containing delivery vehicles, the increased acidity as the endosome ages results in a vehicle with a strong positive charge on the surface. Interactions between the delivery vehicle and the endosomal membrane then result in a membrane fusion event that leads to cytosolic delivery of the payload. For mRNA payloads, the cell's own internal translation processes will then translate the mRNA into the encoded protein. The encoded protein can further undergo postranslation processing, including transportation to a targeted organelle or location within the cell. In the case of an OTC protein, the OTC protein is transported to the mitochondria.

By controlling the composition and concentration of the lipid conjugate, one can control the rate at which the lipid conjugate exchanges out of the lipid formulation and, in turn, the rate at which the lipid formulation becomes fusogenic. In addition, other variables including, e.g., pH, temperature, or ionic strength, can be used to vary and/or control the rate at which the lipid formulation becomes fusogenic. Other methods which can be used to control the rate at which the lipid formulation becomes fusogenic will become apparent to those of skill in the art upon reading this disclosure. Also, by controlling the composition and concentration of the lipid conjugate, one can control the liposomal or lipid particle size.

### Lipid Formulation Manufacture

There are many different methods for the preparation of lipid formulations comprising a nucleic acid. (Curr. Drug Metabol. 2014, 15, 882-892; Chem. Phys. Lipids 2014, 177, 8-18; Int. J. Pharm. Stud. Res. 2012, 3, 14-20). The techniques of thin film hydration, double emulsion, reverse phase evaporation, microfluidic preparation, dual assymetric centrifugation, ethanol injection, detergent dialysis, spontaneous vesicle formation by ethanol dilution, and encapsulation in preformed liposomes are briefly described herein.

### Thin Film Hydration

In Thin Film Hydration (TFH) or the Bangham method, the lipids are dissolved in an organic solvent, then evaporated through the use of a rotary evaporator leading to a thin lipid layer formation. After the layer hydration by an aqueous buffer solution containing the compound to be loaded, Multilamellar Vesicles (MLVs) are formed, which can be reduced in size to produce Small or Large Unilamellar vesicles (LUV and SUV) by extrusion through membranes or by the sonication of the starting MLV.

### Double Emulsion

Lipid formulations can also be prepared through the Double Emulsion technique, which involves lipids dissolution in a water/organic solvent mixture. The organic solution, containing water droplets, is mixed with an excess of aqueous medium, leading to a water-in-oil-in-water (W/O/W) double emulsion formation. After mechanical vigorous shaking, part of the water droplets collapse, giving Large Unilamellar Vesicles (LUVs).

### Reverse Phase Evaporation

The Reverse Phase Evaporation (REV) method also allows one to achieve LUVs loaded with nucleic acid. In this technique a two-phase system is formed by phospholipids dissolution in organic solvents and aqueous buffer. The resulting suspension is then sonicated briefly until the mixture becomes a clear one-phase dispersion. The lipid formulation is achieved after the organic solvent evaporation under reduced pressure. This technique has been used to encapsulate different large and small hydrophilic molecules including nucleic acids.

### Microfluidic Preparation

The Microfluidic method, unlike other bulk techniques, gives the possibility of controlling the lipid hydration process. The method can be classified in continuous-flow microfluidic and droplet-based microfluidic, according to the way in which the flow is manipulated. In the microfluidic hydrodynamic focusing (MHF) method, which operates in a continuous flow mode, lipids are dissolved in isopropyl alcohol which is hydrodynamically focused in a microchannel cross junction between two aqueous buffer streams. Vesicles size can be controlled by modulating the flow rates, thus controlling the lipids solution/buffer dilution process. The method can be used for producing oligonucleotide (ON) lipid formulations by using a microfluidic device consisting of three-inlet and one-outlet ports.

### Dual Asymmetric Centrifugation

Dual Asymmetric Centrifugation (DAC) differs from more common centrifugation as it uses an additional rotation around its own vertical axis. An efficient homogenization is achieved due to the two overlaying movements generated: the sample is pushed outwards, as in a normal centrifuge, and then it is pushed towards the center of the vial due to the additional rotation. By mixing lipids and an NaCl-solution a viscous vesicular phospholipid gel (VPC) is achieved, which is then diluted to obtain a lipid formulation dispersion. The lipid formulation size can be regulated by optimizing DAC speed, lipid concentration and homogenization time.

### Ethanol Injection

The Ethanol Injection (EI) method can be used for nucleic acid encapsulation. This method provides the rapid injection of an ethanolic solution, in which lipids are dissolved, into an aqueous medium containing nucleic acids to be encapsulated, through the use of a needle. Vesicles are spontaneously formed when the phospholipids are dispersed throughout the medium.

### Detergent Dialysis

The Detergent dialysis method can be used to encapsulate nucleic acids. Briefly lipid and plasmid are solubilized in a detergent solution of appropriate ionic strength, after removing the detergent by dialysis, a stabilized lipid formulation is formed. Unencapsulated nucleic acid is then removed by ion-exchange chromatography and empty vesicles by sucrose density gradient centrifugation. The technique is highly sensitive to the cationic lipid content and to the salt concentration of the dialysis buffer, and the method is also difficult to scale.

### Spontaneous Vesicle Formation by Ethanol Dilution

Stable lipid formulations can also be produced through the Spontaneous Vesicle Formation by Ethanol Dilution method in which a stepwise or dropwise ethanol dilution provides the instantaneous formation of vesicles loaded with nucleic acid by the controlled addition of lipid dissolved in ethanol to a rapidly mixing aqueous buffer containing the nucleic acid.

### Encapsulation in Preformed Liposomes

The entrapment of nucleic acids can also be obtained starting with preformed liposomes through two different methods: (1) A simple mixing of cationic liposomes with nucleic acids which gives electrostatic complexes called “lipoplexes”, where they can be successfully used to transfect cell cultures, but are characterized by their low encapsulation efficiency and poor performance in vivo; and (2) a liposomal destabilization, slowly adding absolute ethanol to a suspension of cationic vesicles up to a concentration of 40% v/v followed by the dropwise addition of nucleic acids achieving loaded vesicles; however, the two main steps characterizing the encapsulation process are too sensitive, and the particles have to be downsized.

OTC mRNA Lipid Formulations

The present disclosure provides for lipid formulations comprising an mRNA encoding an enzyme having ornithine transcarbamylase (OTC) activity (OTC mRNA). Following transfection of one or more target cells by the OTC mRNA lipid formulations of the present disclosure, expression of the OTC enzyme encoded by such mRNA will be stimulated and the capability of such target cells to express the OTC enzyme is enhanced. The OTC mRNA can be any suitable mRNA for expressing an OTC enzyme in vivo. In some embodiments, the OTC mRNA encodes a modified OTC enzyme engineered to have increased in vivo stability against cellular degradation and/or increased mitochondrial uptake, including the OTC enzyme of SEQ ID NO: 4.

In a first OTC mRNA-lipid formulation, an OTC mRNA-lipid formulation comprises a compound of Formula (I) and an mRNA encoding an enzyme having OTC activity. In some embodiments the mRNA encodes an OTC enzyme consisting of a sequence having 95% identity to SEQ ID NO: 3. In some embodiments, the mRNA encodes an OTC enzyme consisting of SEQ ID NO: 3. In some embodiments the mRNA encodes an OTC enzyme consisting of a sequence having 95% identity to SEQ ID NO: 4. In some embodiments, the mRNA encodes an OTC enzyme consisting of SEQ ID NO: 4. The compound of Formula I can be selected based on desirable properties including its lipophilicity, potency, selectivity for a specific target cell, in vivo biodegradability, toxicity and immunogenicity profile, and the pKa of the ionizable/protonatable group on the compound of Formula (I).

In some embodiments of the first OTC mRNA-lipid formulation, X⁷ is S. In some embodiments, X⁵ is-C(O)O—, whereby —C(O)O—R⁶ is formed and X⁶ is-C(O)O-whereby —C(O)O—R⁵ is formed. In some embodiments, R⁷ and R⁸ are each independently selected from the group consisting of methyl, ethyl and isopropyl. In some embodiments, L⁵ and L⁶ are each independently a C₁-C₁₀ alkyl. In some embodiments, L⁵ is C₁-C₃ alkyl, and L⁶ is C₁-C₅ alkyl. In some embodiments, L⁶ is C₁-C₂ alkyl. In some embodiments, L⁵ and L⁶ are each a linear C₇ alkyl. In some embodiments, L⁵ and L⁶ are each a linear C₉ alkyl. In some embodiments, R⁵ and R⁶ are each independently an alkenyl. In some embodiments, R⁶ is alkenyl. In some embodiments, R⁶ is C₂-C₉ alkenyl. In some embodiments, the alkenyl comprises a single double bond. In some embodiments, R⁵ and R⁶ are each alkyl. In some embodiments, R⁵ is a branched alkane. In some embodiments, R⁵ and R⁶ are each independently selected from the group consisting of a C₉ alkyl, C₉ alkenyl and C₉ alkynyl. In some embodiments, R⁵ and R⁶ are each independently selected from the group consisting of a C₁₁ alkyl, C₁₁ alkenyl and C₁₁ alkynyl. In some embodiments, R⁵ and R⁶ are each independently selected from the group consisting of a C₇ alkyl, C₇ alkenyl and C₇ alkynyl. In some embodiments, R⁵ is-CH((CH₂)ₚCH₃)₂ or —CH((CH₂)ₚCH₃) ((CH₂)ₚ₋₁CH₃), wherein p is 4-8. In some embodiments, p is 5 and L⁵ is a C₁-C₃ alkyl. In some embodiments, p is 6 and L⁵ is a C₃ alkyl. In some embodiments, p is 7. In some embodiments, p is 8 and L⁵ is a C₁-C₃ alkyl. In some embodiments, R⁵ consists of —CH((CH₂)ₚCH₃) ((CH₂)ₚ₋₁CH₃), wherein p is 7 or 8. In some embodiments, R⁴ is ethylene or propylene. In some embodiments, R⁴ is n-propylene or isobutylene. In some embodiments, L⁷ is absent, R⁴ is ethylene, X⁷ is S and R⁷ and R⁸ are each methyl. In some embodiments, L′ is absent, R⁴ is n-propylene, X⁷ is S and R⁷ and R⁸ are each methyl. In some embodiments, L⁷ is absent, R⁴ is ethylene, X⁷ is S and R⁷ and R⁸ are each ethyl.

In some embodiments of the first OTC mRNA-lipid formulation, X⁷ is S, X⁵ is —C(O)O—, whereby —C(O)O—R⁶ is formed and X⁶ is-C(O)O—, whereby —C(O)O—R⁵ is formed, L⁵ and L⁶ are each independently a linear alkyl C₃-C₇ L⁷ is absent, R⁵ is —CH((CH₂)ₚCH₃)₂, and R⁶ is C₇-C₁₂ alkenyl. In some further embodiments, p is 6 and R⁶ is C₉ alkenyl.

Any mRNA encoding an enzyme having OTC activity is suitable for inclusion in the first OTC mRNA-lipid formulation of the present disclosure. In some embodiments, a suitable mRNA is a wild-type human OTC mRNA of sequence SEQ ID NO: 3. Preferably, the OTC mRNA has low immunogenicity, high in vivo stability, and high translation efficiency. In some embodiments, the OTC mRNA is expressible in human hepatocytes. In some embodiments, the OTC mRNA has a coding region that is codon-optimized. In some embodiments, the OTC mRNA comprises modified uridine nucleotides. In some embodiments, the modified uridine nucleotides are N¹-methylpseudouridine or 5-methoxyuridine. In some embodiments, the modified uridine nucleotides are 5-methoxyuridine. In some embodiments, the OTC mRNA can be any of the OTC mRNA constructs described herein.

In some embodiments of the first OTC mRNA-lipid formulation, the mRNA comprises an open reading frame (ORF or coding region) selected from SEQ ID NOs: 254-258. In some embodiments, the mRNA comprises an ORF having a sequence of SEQ ID NO: 254. In some embodiments, the mRNA comprises an ORF having a sequence of SEQ ID NO: 255. In some embodiments, the mRNA comprises an ORF having a sequence of SEQ ID NO: 256. In some embodiments, the mRNA comprises an ORF having a sequence of SEQ ID NO: 257. In some embodiments, the mRNA comprises an ORF having a sequence of SEQ ID NO: 258. In some embodiments, the mRNA comprises a sequence having about 85% identity to a sequence selected from SEQ ID NOs: 73, 119, and 251-253. In some embodiments, the mRNA comprises a sequence having about 90% identity to a sequence selected from SEQ ID NOs: 73, 119, and 251-253. In some embodiments, the mRNA comprises a sequence having about 95% identity to a sequence selected from SEQ ID NOs: 73, 119, and 251-253. In some embodiments, the mRNA comprises a sequence having about 96% identity to a sequence selected from SEQ ID NOs: 73, 119, and 251-253. In some embodiments, the mRNA comprises a sequence having about 97% identity to a sequence selected from SEQ ID NOs: 73, 119, and 251-253. In some embodiments, the mRNA comprises a sequence having about 98% identity to a sequence selected from SEQ ID NOs: 73, 119, and 251-253. In some embodiments, the mRNA comprises a sequence having about 99% identity to a sequence selected from SEQ ID NOs: 73, 119, and 251-253. In some embodiments, the mRNA comprises a sequence having about 99.5% identity to a sequence selected from SEQ ID NOs: 73, 119, and 251-253. In some embodiments, the mRNA comprises a sequence selected from SEQ ID NOS: 73, 119, and 251-253. In some embodiments, the mRNA comprises a sequence selected from SEQ ID NO: 1.

In any of the embodiments of the first OTC mRNA-lipid formulation, the OTC mRNA-lipid formulation comprises lipid nanoparticles. In some embodiments, the lipid nanoparticles completely encapsulate the OTC mRNA.

In some embodiments, the lipid nanoparticles have an average particle size of less than about 100 nm. In some embodiments, the lipid nanoparticles have an average particles size of about 55 to about 85 nm. In some embodiments, the lipid nanoparticles encapsulate at least about 50% of the mRNA. In some embodiments, the lipid nanoparticles encapsulate at least about 85% of the mRNA. In some embodiments, the lipid nanoparticles have greater than about 90% encapsulation efficiency. In some embodiments, the lipid nanoparticles have greater than about 95% encapsulation efficiency.

In a second OTC mRNA-lipid formulation, an OTC mRNA-lipid formulation comprises a compound selected from:

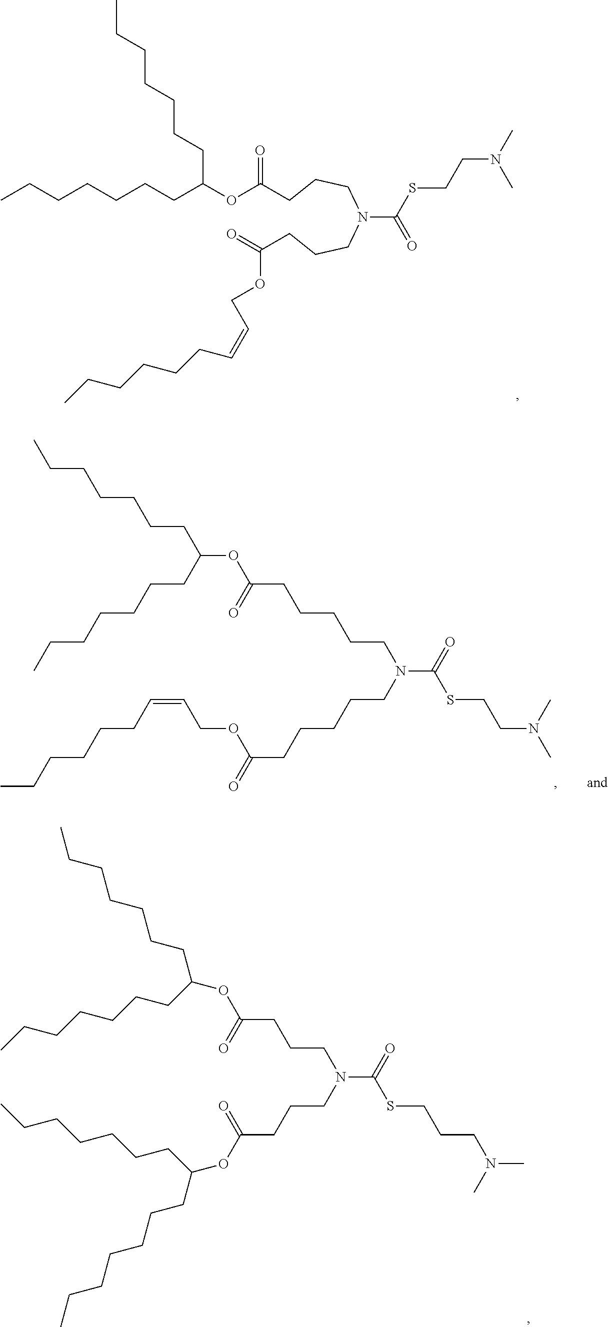

or a pharmaceutically acceptable salt thereof and an mRNA encoding an enzyme having OTC activity.

Any mRNA encoding an enzyme having OTC activity is suitable for inclusion in the second OTC mRNA-lipid formulation of the present disclosure. In some embodiments, a suitable mRNA is a wild-type human OTC mRNA of SEQ ID NO: 3. In some embodiments the mRNA encodes an OTC enzyme consisting of a sequence having 95% identity to SEQ ID NO: 3. In some embodiments, the mRNA encodes an OTC enzyme consisting of SEQ ID NO: 3. In some embodiments the mRNA encodes an OTC enzyme consisting of a sequence having 95% identity to SEQ ID NO: 4. In some embodiments, the mRNA encodes an OTC enzyme consisting of SEQ ID NO: 4. Preferably, the OTC mRNA has low immunogenicity, high in vivo stability, and high translation efficiency. In some embodiments, the OTC mRNA is expressible in human hepatocytes. In some embodiments, the OTC mRNA has a coding region that is codon-optimized. In some embodiments, the OTC mRNA comprises modified uridine nucleotides. In some embodiments, the modified uridine nucleotides are N¹-methylpseudouridine or 5-methoxyuridine. In some embodiments, the modified uridine nucleotides are 5-methoxyuridine. In some embodiments, the OTC mRNA can be any of the OTC mRNA constructs described herein.

In some embodiments of the second OTC mRNA-lipid formulation, the mRNA comprises an open reading frame (ORF or coding region) selected from SEQ ID Nos: 254-258. In some embodiments, the mRNA comprises an ORF having a sequence of SEQ ID NO: 254. In some embodiments, the mRNA comprises an ORF having a sequence of SEQ ID NO: 255. In some embodiments, the mRNA comprises an ORF having a sequence of SEQ ID NO: 256. In some embodiments, the mRNA comprises an ORF having a sequence of SEQ ID NO: 257. In some embodiments, the mRNA comprises an ORF having a sequence of SEQ ID NO: 258. In some embodiments, the mRNA comprises a sequence having about 85% identity to a sequence selected from SEQ ID NOS: 73, 119, and 251-253. In some embodiments, the mRNA comprises a sequence having about 90% identity to a sequence selected from SEQ ID NOs: 73, 119, and 251-253. In some embodiments, the mRNA comprises a sequence having about 95% identity to a sequence selected from SEQ ID NOs: 73, 119, and 251-253. In some embodiments, the mRNA comprises a sequence having about 96% identity to a sequence selected from SEQ ID NOs: 73, 119, and 251-253. In some embodiments, the mRNA comprises a sequence having about 97% identity to a sequence selected from SEQ ID NOs: 73, 119, and 251-253. In some embodiments, the mRNA comprises a sequence having about 98% identity to a sequence selected from SEQ ID NOs: 73, 119, and 251-253. In some embodiments, the mRNA comprises a sequence having about 99% identity to a sequence selected from SEQ ID NOs: 73, 119, and 251-253. In some embodiments, the mRNA comprises a sequence having about 99.5% identity to a sequence selected from SEQ ID NOs: 73, 119, and 251-253. In some embodiments, the mRNA comprises a sequence selected from SEQ ID NOS: 73, 119, and 251-253. In some embodiments, the mRNA comprises a sequence selected from SEQ ID NO: 1.

In any of the embodiments of the second OTC mRNA-lipid formulation, the OTC mRNA-lipid formulation comprises lipid nanoparticles. In some embodiments, the lipid nanoparticles completely encapsulate the OTC mRNA.

In some embodiments, the lipid nanoparticles have an average particle size of less than about 100 nm. In some embodiments, the lipid nanoparticles have an average particles size of about 55 nm to about 85 nm. In some embodiments, the lipid nanoparticles encapsulate at least about 50% of the mRNA. In some embodiments, the lipid nanoparticles encapsulate at least about 85% of the mRNA. In some embodiments, the lipid nanoparticles have greater than about 90% encapsulation efficiency.

In some embodiments, either the first or second OTC mRNA-lipid formulation further comprises a helper lipid. In some embodiments, the helper lipid is selected from the group consisting of neutral and anionic lipids. In some embodiments, the helper lipid is selected from the group consisting of dipalmitoyl phosphatidylcholine (DPPC), phosphatidylcholine (PC), dioleoylphosphatidyl ethanolamine (DOPE), dimyristoylphosphatidyl choline (DMPC), distearoylphosphatidyl choline, and dimyristoylphosphatidyl glycerol (DMPG). In some embodiments, the noncationic lipid is distearoylphosphatidylcholine (DSPC).

In some embodiments, either the first or second OTC mRNA-lipid formulation further comprises cholesterol.

In some embodiments, either the first or second OTC mRNA-lipid formulation further comprises a polyethylene glycol (PEG)-lipid conjugate. In some embodiments, the PEG-lipid conjugate is PEG-DMG. In some embodiments, the PEG-DMG is PEG2000-DMG.

In some embodiments, the lipid portion (meaning the total amount of lipids in the formulation) of either the first or second OTC mRNA-lipid formulation comprises about 48 mol % to about 66 mol % of the cationic lipid, about 2 mol % to about 12 mol % DSPC, about 25 to about 42 mol % cholesterol, and about 0.5 mol % to about 3 mol % PEG2000-DMG.

In some embodiments, the lipid portion of either the first or second OTC mRNA-lipid formulation comprises about 55 mol % to about 61 mol % of the cationic lipid, about 5 mol % to about 9 mol % DSPC, about 29 mol % to about 38 mol % cholesterol, and about 1 mol % to about 2 mol % PEG2000-DMG.

In some embodiments, the lipid portion of either the first or second OTC mRNA-lipid formulation comprises about 56 mol % to about 60 mol % of the cationic lipid, about 6 mol % to about 8 mol % DSPC, about 31 mol % to about 34 mol % cholesterol, and about 1.25 mol % to about 1.75 mol % PEG2000-DMG.

In some embodiments, either the first or second OTC mRNA-lipid formulation has a total lipid: mRNA weight ratio of about 50:1 to about 10:1. In some embodiments, either the first or second OTC mRNA-lipid formulation has a total lipid: mRNA weight ratio of about 40:1 to about 20:1. In some embodiments, either the first or second OTC mRNA-lipid formulation has a total lipid: mRNA weight ratio of about 35:1 to about 25:1. In some embodiments, either the first or second OTC mRNA-lipid formulation has a total lipid: mRNA weight ratio of about 28:1 to about 32:1. In some embodiments, either the first or second OTC mRNA-lipid formulation has a total lipid: mRNA weight ratio of about 29:1 to about 31:1.

### Pharmaceutical Compositions and Methods of Treatment

To facilitate expression of mRNA in vivo, the nucleic acid lipid formulation delivery vehicles described herein can be combined with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition. Preferably, the nucleic acid lipid formulation is an OTC mRNA lipid nanoparticle formulation as described herein. In some embodiments, the pharmaceutical composition further comprises pharmaceutically acceptable excipients. Pharmaceutical compositions disclosed herein preferably facilitate expression of OTC mRNA in vivo.

The lipid formulations and pharmaceutical compositions of the present disclosure may be administered and dosed in accordance with current medical practice, taking into account the clinical condition of the subject, the site and method of administration, the scheduling of administration, the subject's age, sex, body weight and other factors relevant to clinicians of ordinary skill in the art. The “effective amount” for the purposes herein may be determined by such relevant considerations as are known to those of ordinary skill in experimental clinical research, pharmacological, clinical and medical arts. In some embodiments, the amount administered is effective to achieve at least some stabilization, improvement or elimination of symptoms and other indicators as are selected as appropriate measures of disease progress, regression or improvement by those of skill in the art. For example, a suitable amount and dosing regimen is one that causes at least transient protein (e.g., enzyme) production.

In some embodiments, the pharmaceutical compositions described are administered systemically. Suitable routes of administration include, for example, oral, rectal, vaginal, transmucosal, pulmonary including intratracheal or inhaled, or intestinal administration; parenteral delivery, including intradermal, transdermal (topical), intramuscular, subcutaneous, intramedullary injections, as well as intrathecal, direct intraventricular, intravenous, intraperitoneal, or intranasal. In particular embodiments, the intramuscular administration is to a muscle selected from the group consisting of skeletal muscle, smooth muscle and cardiac muscle. In some embodiments, the pharmaceutical composition is administered intravenously. In some embodiments, the administration results in delivery of the mRNA to a hepatocyte (i.e., liver cell). In some embodiments, the administration shows a selectivity towards hepatocytes over other types of liver cells (e.g., stellate cells, etc.).

Pharmaceutical compositions may be administered to any desired tissue. In some embodiments, the OTC mRNA delivered is expressed in a tissue different from the tissue in which the lipid formulation or pharmaceutical composition was administered. In preferred embodiments, OTC mRNA is delivered and expressed in the liver.

The pharmaceutical compositions disclosed herein can be formulated using one or more excipients to: (1) increase stability; (2) increase cell transfection; (3) permit a sustained or delayed release (e.g., from a depot formulation of the polynucleotide, primary construct, or mRNA); (4) alter the biodistribution (e.g., target the polynucleotide, primary construct, or mRNA to specific tissues or cell types); (5) increase the translation of encoded protein in vivo; and/or (6) alter the release profile of encoded protein in vivo.

The pharmaceutical compositions described herein may be prepared by any method known or hereafter developed in the art of pharmacology. In general, such preparatory methods include the step of associating the active ingredient (i.e., nucleic acid) with an excipient and/or one or more other accessory ingredients. A pharmaceutical composition in accordance with the present disclosure may be prepared, packaged, and/or sold in bulk, as a single unit dose, and/or as a plurality of single unit doses.

Pharmaceutical compositions may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired.

In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, liposomes, lipid nanoparticles, polymers, lipoplexes, core-shell nanoparticles, peptides, proteins, cells transfected with primary DNA construct, or mRNA (e.g., for transplantation into a subject), hyaluronidase, nanoparticle mimics and combinations thereof.

Accordingly, the pharmaceutical compositions described herein can include one or more excipients, each in an amount that together increases the stability of the nucleic acid in the lipid formulation, increases cell transfection by the nucleic acid, increases the expression of the encoded protein, and/or alters the release profile of encoded proteins. Further, the mRNA of the present disclosure may be formulated using self-assembled nucleic acid nanoparticles.

Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the embodiments of the present disclosure, except insofar as any conventional excipient medium may be incompatible with a substance or its derivatives, such as by producing any undesirable biological effect or otherwise interacting in a deleterious manner with any other component(s) of the pharmaceutical composition.

A dosage form of the composition of this disclosure can be solid, which can be reconstituted in a liquid prior to administration. The solid can be administered as a powder. The solid can be in the form of a capsule, tablet, or gel. In some embodiments, the pharmaceutical composition comprises a nucleic acid lipid formulation that has been lyophilized.

In a preferred embodiment, the dosage form of the pharmaceutical compositions described herein can be a liquid suspension of OTC mRNA lipid nanoparticles described herein. In some embodiments, the liquid suspension is in a buffered solution. In some embodiments, the buffered solution comprises a buffer selected from the group consisting of HEPES, MOPS, TES, and TRIS. In some embodiments, the buffer has a pH of about 7.4. In some preferred embodiments, the buffer is HEPES. In some further embodiments, the buffered solution further comprises a cryoprotectant. In some embodiments, the cryoprotectant is selected from a sugar and glycerol or a combination of a sugar and glycerol. In some embodiments, the sugar is a dimeric sugar. In some embodiments, the sugar is sucrose. In some preferred embodiments, the buffer comprises HEPES, sucrose, and glycerol at a pH of 7.4. In some embodiments, the suspension is frozen during storage and thawed prior to administration. In some embodiments, the suspension is frozen at a temperature below about 70° C. In some embodiments, the suspension is diluted with sterile water during intravenous administration. In some embodiments, intravenous administration comprises diluting the suspension with about 2 volumes to about 6 volumes of sterile water. In some embodiments, the suspension comprises about 0.1 mg to about 3.0 mg OTC mRNA/mL, about 15 mg/mL to about 25 mg/mL of an ionizable cationic lipid, about 0.5 mg/mL to about 2.5 mg/mL of a PEG-lipid, about 1.8 mg/mL to about 3.5 mg/mL of a helper lipid, about 4.5 mg/mL to about 7.5 mg/mL of a cholesterol, about 7 mg/mL to about 15 mg/mL of a buffer, about 2.0 mg/mL to about 4.0 mg/mL of NaCl, about 70 mg/mL to about 110 mg/mL of sucrose, and about 50 mg/mL to about 70 mg/mL of glycerol. In some embodiments, a lyophilized OTC-mRNA lipid nanoparticle formulation can be resuspended in a buffer as described herein.

The pharmaceutical compositions of this disclosure may further contain as pharmaceutically acceptable carriers substances as required to approximate physiological conditions, such as pH adjusting and buffering agents, tonicity adjusting agents, and wetting agents, for example, sodium acetate, sodium lactate, sodium chloride, potassium chloride, calcium chloride, sorbitan monolaurate, triethanolamine oleate, and mixtures thereof. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

In certain embodiments of the disclosure, the mRNA-lipid formulation may be administered in a time release formulation, for example in a composition which includes a slow release polymer. The active agent can be prepared with carriers that will protect against rapid release, for example a controlled release vehicle such as a polymer, microencapsulated delivery system, or a bioadhesive gel. Prolonged delivery of the mRNA, in various compositions of the disclosure can be brought about by including in the composition agents that delay absorption, for example, aluminum monostearate hydrogels and gelatin.

Following administration of the composition to the subject, the protein product encoded by the mRNA (e.g., a functional OTC protein or enzyme) is detectable in the target tissues for at least about one to seven days or longer. The amount of protein product necessary to achieve a therapeutic effect will vary depending on the severity of ornithine transcarbamylase deficiency or other disorder being treated and the condition of the patient. For example, the protein product may be detectable in the target tissues at a concentration (e.g., a therapeutic concentration) of at least about 0.025-1.5 μg/ml (e.g., at least about 0.050 μg/ml, at least about 0.075 μg/ml, at least about 0.1 μg/ml, at least about 0.2 μg/ml, at least about 0.3 μg/ml, at least about 0.4 μg/ml, at least about 0.5 μg/ml, at least about 0.6 μg/ml, at least about 0.7 μg/ml, at least about 0.8 μg/ml, at least about 0.9 μg/ml, at least about 1.0 μg/ml, at least about 1.1 μg/ml, at least about 1.2 μg/ml, at least about 1.3 μg/ml, at least about 1.4 μg/ml, or at least about 1.5 μg/ml), for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 days or longer following administration of the composition to the subject.

In some embodiments, the compositions of the disclosure are administered to a subject such that a OTC mRNA concentration of at least about 0.05 mg/kg, at least about 0.1 mg/kg, at least about 0.5 mg/kg, at least about 1.0 mg/kg, at least about 2.0 mg/kg, at least about 3.0 mg/kg, at least about 4.0 mg/kg, at least about 5.0 mg/kg of body weight is administered in a single dose or as part of single treatment cycle. In some embodiments, the compositions of the disclosure are administered to a subject such that a total amount of at least about 0.1 mg, at least about 0.5 mg, at least about 1.0 mg, at least about 2.0 mg, at least about 3.0 mg, at least about 4.0 mg, at least about 5.0 mg, at least about 6.0 mg, at least about 7.0 mg, at least about 8.0 mg, at least about 9.0 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 110 mg, at least about 115 mg, at least about 120 mg, or at least about 125 mg OTC mRNA is administered in one or more doses up to a maximum dose of about 300 mg, about 350 mg, about 400 mg, about 450 mg, or about 500 mg OTC mRNA.

The compositions and polynucleotides of the present disclosure may be used to treat a subject who is suffering from or susceptible to ornithine transcarbamylase (OTC) deficiency. OTC is a homotrimeric mitochondrial enzyme which is expressed almost exclusively in the liver and which encodes a precursor OTC protein that is cleaved in two steps upon incorporation into the mitochondrial matrix. (Horwich A L., et al. Cell 1986; 44:451-459). OTC deficiency is a genetic disorder which results in a mutated and biologically inactive form of the enzyme ornithine transcarbamylase. OTC deficiency often becomes evident in the first few days of life, typically after protein ingestion. In the classic severe form of OTC deficiency, within the first days of life patients present with lethargy, convulsions, coma and severe hyperammonemia, which quickly leads to a deteriorating and fatal outcome absent appropriate medical intervention. (Morrish S., et al., Genetics for Pediatricians; Remedica, Cold Spring Harbor Laboratory (2005)). If improperly treated or if left untreated, complications from OTC deficiency may include developmental delay and mental retardation. OTC deficient subjects may also present with progressive liver damage, skin lesions, and brittle hair. In some affected individuals, signs and symptoms of OTC deficiency may be less severe, and may not appear until later in life.

The OTC gene, which is located on the short arm of the X chromosome within band Xp21.1, spans more than 85 kb and is comprised of 10 exons encoding a protein of 1062 amino acids. (Lindgren V., et al. Science 1984; 226:6987700; Horwich, A L., et al. Science 224:1068-1074, 1984; Horwich, A L. et al., Cell 44:451-459, 1986; Hata, A., et al., J. Biochem. 100:717-725, 1986, which are incorporated herein by reference). The OTC enzyme catalyzes the conversion or ornithine and carbamoyl phosphate to citrulline. Since OTC is on the X chromosome, females are primarily carriers while males with nonconservative mutations rarely survive past 72 hours of birth.

In some embodiments, a pharmaceutical composition of the present disclosure is administered to a subject once per month. In some embodiments, a pharmaceutical composition of the present disclosure is administered to a subject twice per month. In some embodiments, a pharmaceutical composition of the present disclosure is administered to a subject three times per month. In some embodiments, a pharmaceutical composition of the present disclosure is administered to a subject four times per month.

In healthy subjects, OTC is expressed almost exclusively in hepatocellular mitochondria. Although not expressed in the brain of healthy subjects, OTC deficiency can lead to neurological disorders. For example, one of the usual symptoms of OTC deficiency, which is heterogeneous in its presentation, is hyperammonaemic coma (Gordon, N., Eur J Paediatr Neural 2003; 7:115-121.).

OTC deficiency is heterogeneous, with over 200 unique mutations reported and large deletions that account for approximately 10-15% of all mutations, while the remainder generally comprises missense point mutations with smaller numbers of nonsense, splice-site and small deletion mutations. (Morrish A., et al.) The phenotypic manifestations of OTC deficiency is also highly heterogeneous, which can range from acute neonatal hyperammonemic coma to asymptomatic hemizygous adults. (Gordon N. Eur J. Paediatr. Neurol. 2003; 7:115-121). Those mutations that result in severe and life threatening neonatal disease are clustered in important structural and functional domains in the interior of the protein at sites of enzyme activity or at the interchain surface, while mutations associated with late-onset disease are located on the protein surface (Morrish A., et al.) Patients with milder or partial forms of OTC deficiency may have onset of disease later in life, which may present as recurrent vomiting, neurobehavioral changes or seizures associated with hyperammonemia.

Alternatively, the compositions of the present disclosure may be administered in a local rather than systemic manner, for example, via injection of the pharmaceutical composition directly into a targeted tissue, preferably in a depot or sustained release formulation. Local delivery can be affected in various ways, depending on the tissue to be targeted. For example, aerosols containing compositions of the present disclosure can be inhaled (for nasal, tracheal, or bronchial delivery); compositions of the present disclosure can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection. Formulations containing compositions of the present disclosure complexed with therapeutic molecules or ligands can even be surgically administered, for example in association with a polymer or other structure or substance that can allow the compositions to diffuse from the site of implantation to surrounding cells. Alternatively, they can be applied surgically without the use of polymers or supports.

According to the present disclosure, a therapeutically effective dose of the provided composition, when administered regularly, results in an increased OTC protein expression or activity level in a subject as compared to a baseline OTC protein expression or activity level before treatment. Typically, the OTC protein expression or activity level is measured in a biological sample obtained from the subject such as blood, plasma or serum, urine, or solid tissue extracts. The baseline level can be measured immediately before treatment. In some embodiments, administering a pharmaceutical composition described herein results in an increased OTC protein expression or activity level in a biological sample (e.g., plasma/serum or urine) by at least about 10%, 20%, 30%, 40%, 50%, 60%, 70%, 80%, 90%, or 95% as compared to a baseline level before treatment. In some embodiments, administering the provided composition results in an increased OTC protein expression or activity level in a biological sample (e.g., plasma/serum or urine) by at least about 10%, 20%, 30%, 40%, 50%, 60%, 70%, 80%, 90%, or 95% as compared to a baseline level before treatment for at least about 24 hours, at least about 48 hours, at least about 72 hours, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, or at least about 15 days.

In some embodiments, a therapeutically effective dose of the provided composition, when administered regularly, results in an increased citrulline production in a subject as compared to a baseline citrulline production before treatment. Typically, the citrulline level before or after the treatment may be measured in a biological sample obtained from the subject such as, blood, plasma or serum, urine, or solid tissue extracts. In some embodiments, treatment according to the present disclosure results in an increase of the citrulline level in a biological sample (e.g., plasma, serum, or urine) by at least about 10%, 20%, 30%, 40%, 50%, 60%, 70%, 80%, 90%, 1-fold, 1.5-fold, 2-fold, 2.5-fold, or 3-fold as compared to the baseline citrulline level, respectively.

According to the present disclosure, a therapeutically effective dose of the provided composition, when administered regularly, results in reduction of at least one symptom or feature of the OTC deficiency, including in intensity, severity, or frequency or the symptom has delayed onset. In some embodiments, a therapeutically effective dose of the provided composition, when administered regularly, results in a reduced orotic acid level in a subject as compared to a baseline orotic acid level before treatment. In some embodiments, a therapeutically effective dose of the provided composition, when administered regularly, results in a reduced ammonia level in a subject as compared to a baseline ammonia level before treatment. In some embodiments, a therapeutically effective dose of the provided composition, when administered regularly, results in a reduced glutamine level in a subject as compared to a baseline glutamine level before treatment.

Typically, the orotic acid, ammonia or glutamine level before or after the treatment may be measured in a biological sample obtained from the subject such as, blood, plasma, serum, urine, or solid tissue extracts. The baseline orotic acid, ammonia or glutamine level can be measured immediately before treatment. In some embodiments, treatment according to the present disclosure results in a reduction of the orotic acid, ammonia, or glutamine level in a biological sample (e.g., blood, serum, or urine) obtained from the subject by at least about 10%, 20%, 30%, 40%, 50%, 60%, 70%, 80%, 90%, or 95% as compared to the baseline orotic acid, ammonia, or glutamine level, respectively. In some embodiments, a therapeutically effective dose of the provided composition, when administered regularly, results in a reduced plasma ammonia level to less than about 500 μmol/L, 400 μmol/L, 300 μmol/L, 200 μmol/L, 150 μmol/L, or 100 μmol/L. In some embodiments, a therapeutically effective dose of the provided composition, when administered regularly, results in a reduced plasma glutamine level to less than about 800 μmol/L, 700 μmol/L, or 600 μmol/L. In some embodiments, a therapeutically effective dose of the provided composition, when administered regularly, results in a reduced urinary orotic acid level to less than about 20 μmol/mmol creatinine, 15 μmol/mmol creatinine, or 10 μmol/mmol creatinine.

In some embodiments, administering the provided composition results in an increased OTC protein level in the liver of a subject as compared to a baseline level before treatment. Typically, the baseline level is measured immediately before treatment. In some embodiments, administering the provided composition results in an increased OTC protein level in the liver by at least about 10%, 20%, 30%, 40%, 50%, 60%, 70%, 80%, 90%, or 95% as compared to a baseline level before treatment. In some embodiments, administering the provided composition results in an increased OTC protein level in the liver as compared to a OTC protein level in the liver of subjects who are not treated.

In some embodiments, administering the provided composition results in an increased level of OTC protein in a liver cell (e.g., a hepatocyte) of a subject as compared to a baseline level before treatment. Typically, the baseline level is measured immediately before treatment. In some embodiments, administering the provided composition results in an increased OTC protein level in the liver cell by at least about 10%, 20%, 30%, 40%, 50%, 60%, 70%, 80%, 90%, or 95% as compared to a baseline level before treatment. In some embodiments, administering the provided composition results in an increased OTC protein level in a liver cell as compared to the OTC protein level a liver cell of subjects who are not treated.

In some embodiments, administering the provided composition results in an increased OTC protein level in plasma or serum of a subject as compared to a baseline level before treatment. Typically, the baseline level is measured immediately before treatment. In some embodiments, administering the provided composition results in an increased OTC protein level in plasma or serum by at least about 10%, 20%, 30%, 40%, 50%, 60%, 70%, 80%, 90%, or 95% as compared to a baseline level before treatment. In some embodiments, administering the provided composition results in an increased OTC protein level in plasma or serum as compared to an OTC protein level in plasma or serum of subjects who are not treated.

In some embodiments, administering the provided composition results in increased OTC enzyme activity in a biological sample from a subject as compared to the baseline level before treatment. Typically, the baseline level is measured immediately before treatment. Biological samples include, for example, whole blood, serum, plasma, urine and tissue samples (e.g., liver). In some embodiments, administering the provided composition results in an increased OTC enzyme activity by at least about 10%, 20%, 30%, 40%, 50%, 60%, 70%, 80%, 90%, or 95% as compared to a baseline level immediately before treatment. In some embodiments, administering the provided composition results in an increased OTC enzyme activity as compared to OTC enzyme activity in subjects who are not treated.

In some embodiments, provided herein are methods of administering a therapeutic intervention to a subject suspected of having an ornithine transcarbamylase (OTC) enzyme deficiency including measuring in the subject a level of OTC enzyme activity indicator and administering to the subject a therapeutic intervention when an OTC enzyme indicator signals deficient OTC enzyme activity. In some embodiments, the subject is a human. In some embodiments, the subject is an adult. In some embodiments, the subject is a human neonate. In some embodiments, the therapeutic intervention includes administering a composition according to any of the various embodiments described herein. In some embodiments, measuring a level of an OTC enzyme activity indicator is selected from measuring OTC enzyme levels in a liver biopsy, measuring nitrogen levels in a blood sample from the subject, measuring citrulline levels in a liver biopsy, and measuring orotic acid in a urinary sample from the subject. In some embodiments, measuring a level of an OTC enzyme activity indicator includes measuring OTC enzyme levels in a liver biopsy. In some embodiments, measuring a level of an OTC enzyme activity indicator includes measuring nitrogen levels in a blood sample from the subject, measuring citrulline levels in a liver biopsy. In some embodiments, measuring a level of an OTC enzyme activity indicator includes measuring orotic acid in a urinary sample from the subject.

In some embodiments, provided herein are methods of treating OTC deficiency in a subject identified as suffering from OTC deficiency. In some embodiments, methods provided herein include administering to the subject any composition provided herein. In some embodiments, an OTC enzyme including a sequence of SEQ ID NO:3 is expressed in the subject. In some embodiments, an OTC enzyme including a sequence of SEQ ID NO:4 is expressed in the subject.

### Combinations

The OTC mRNA, formulations thereof, or encoded OTC proteins described herein may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. Preferably, the methods of treatment of the present disclosure encompass the delivery of pharmaceutical, prophylactic, diagnostic, or imaging compositions in combination with agents that may improve their bioavailability, reduce and/or modify their metabolism, inhibit their excretion, and/or modify their distribution within the body. As a non-limiting example, mRNA disclosed herein and preferably an mRNA sequence comprising SEQ ID NO: 251 encoding a modified OTC protein of SEQ ID NO: 4 may be used in combination with a pharmaceutical agent for the treatment of OTC deficiency. The pharmaceutical agent includes, but is not limited to one or more of: sodium phenylbutyrate, glycerol phenylbutyrate (marketed e.g., as Ravicti®), sodium phenylacetate, sodium benzoate, arginine, citrulline, Multiple vitamins, calcium supplements or combined with a low protein/high caloric diet regimen. In general, it is expected that agents utilized in combination with the presently disclosed OTC mRNA and formulations thereof be utilized at levels that do not exceed the levels at which they are utilized individually. In some embodiments, the levels utilized in combination will be lower than those utilized individually. In one embodiment, the combinations, each or together may be administered according to the split dosing regimens as are known in the art.

### Definitions

At various places in the present specification, substituents of compounds of the present disclosure are disclosed in groups or in ranges. It is specifically intended that the present disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

The phrases “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically engineered animal, or a clone.

The terms “approximately” or “about,” as applied to one or more values of interest, refers to a value that is similar to a stated reference value. In certain embodiments, the term “approximately” or “about” refers to a range of values that fall within +/−10% of the recited value unless otherwise stated or otherwise evident from the context (except where such number would exceed 100% of a possible value).

The terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

The term “acyl,” as used herein, represents a hydrogen or an alkyl group (e.g., a haloalkyl group), as defined herein, that is attached to the parent molecular group through a carbonyl group, as defined herein, and is exemplified by formyl (i.e., a carboxyaldehyde group), acetyl, trifluoroacetyl, propionyl, butanoyl and the like. Exemplary unsubstituted acyl groups include from 1 to 7, from 1 to 11, or from 1 to 21 carbons. In some embodiments, the alkyl group is further substituted with 1, 2, 3, or 4 substituents as described herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, 2-butenyl, and the like. Alkenyls include both cis and trans isomers. Alkenyl groups may be optionally substituted with 1, 2, 3, or 4 substituent groups that are selected, independently, from amino, aryl, cycloalkyl, or heterocyclyl (e.g., heteroaryl), as defined herein, or any of the exemplary alkyl substituent groups described herein.

The term “alkoxy” represents a chemical substituent of formula-OR, where R is a C₁₋₂₀ alkyl group (e.g., C₁₋₆ or C₁₋₁₀ alkyl), unless otherwise specified. Exemplary alkoxy groups include methoxy, ethoxy, propoxy (e.g., n-propoxy and isopropoxy), t-butoxy, and the like. In some embodiments, the alkyl group can be further substituted with 1, 2, 3, or 4 substituent groups as defined herein (e.g., hydroxy or alkoxy).

The term “alkoxyalkyl” represents an alkyl group that is substituted with an alkoxy group. Exemplary unsubstituted alkoxyalkyl groups include between 2 to 40 carbons (e.g., from 2 to 12 or from 2 to 20 carbons, such as C₁₋₆ alkoxy-C₁₋₆ alkyl, C₁₋₁₀ alkoxy-C₁₋₁₀ alkyl, or C₁₋₂₀ alkoxy-C₁₋₂₀ alkyl). In some embodiments, the alkyl and the alkoxy each can be further substituted with 1, 2, 3, or 4 substituent groups as defined herein for the respective group.

The term “alkoxycarbonyl,” as used herein, represents an alkoxy, as defined herein, attached to the parent molecular group through a carbonyl atom (e.g., —C(O)—OR, where R is H or an optionally substituted C₁₋₆, C₁₋₁₀, or C₁₋₂₀ alkyl group). Exemplary unsubstituted alkoxycarbonyl include from 1 to 21 carbons (e.g., from 1 to 11 or from 1 to 7 carbons). In some embodiments, the alkoxy group is further substituted with 1, 2, 3, or 4 substituents as described herein.

The term “alkoxycarbonylalkyl,” as used herein, represents an alkyl group, as defined herein, that is substituted with an alkoxycarbonyl group, as defined herein (e.g., -alkyl-C(O)—OR, where R is an optionally substituted C₁₋₂₀, C₁₋₁₀, or C₁₋₆ alkyl group). Exemplary unsubstituted alkoxycarbonylalkyl include from 3 to 41 carbons (e.g., from 3 to 10, from 3 to 13, from 3 to 17, from 3 to 21, or from 3 to 31 carbons, such as C₁₋₆ alkoxycarbonyl-C₁₋₆ alkyl, C₁₋₁₀ alkoxycarbonyl-C₁₋₁₀ alkyl, or C₁₋₂₀ alkoxycarbonyl-C₁₋₂₀ alkyl). In some embodiments, each alkyl and alkoxy group is further independently substituted with 1, 2, 3, or 4 substituents as described herein (e.g., a hydroxy group).

The term “alkoxycarbonylalkenyl,” as used herein, represents an alkenyl group, as defined herein, that is substituted with an alkoxycarbonyl group, as defined herein (e.g., -alkenyl-C(O)—OR, where R is an optionally substituted C₁₋₂₀, C₁₋₁₀, or C₁₋₆ alkyl group). Exemplary unsubstituted alkoxycarbonylalkenyl include from 4 to 41 carbons (e.g., from 4 to 10, from 4 to 13, from 4 to 17, from 4 to 21, or from 4 to 31 carbons, such as C₁₋₆ alkoxycarbonyl-C₂-6 alkenyl, C₁₋₁₀ alkoxycarbonyl-C₂₋₁₀ alkenyl, or C₁₋₂₀ alkoxycarbonyl-C₂₋₂₀ alkenyl). In some embodiments, each alkyl, alkenyl, and alkoxy group is further independently substituted with 1, 2, 3, or 4 substituents as described herein (e.g., a hydroxy group).

The term “alkyl,” as used herein, is inclusive of both straight chain and branched chain saturated groups from 1 to 20 carbons (e.g., from 1 to 10 or from 1 to 6), unless otherwise specified. Alkyl groups are exemplified by methyl, ethyl, n- and iso-propyl, n-, sec-, iso- and tert-butyl, neopentyl, and the like, and may be optionally substituted with one, two, three, or, in the case of alkyl groups of two carbons or more, four substituents independently selected from the group consisting of: (1) C₁₋₆ alkoxy; (2) C₁₋₆ alkylsulfinyl; (3) amino, as defined herein (e.g., unsubstituted amino (i.e., —NH₂) or a substituted amino (i.e., —N(R^{N1})₂, where R^{N1} is as defined for amino); (4) —C(O)O— or —OC(O)-aryl-C₁₋₆ alkoxy; (5) azido; (6) halo; (7) (C₂₋₉ heterocyclyl)oxy; (8) hydroxy, optionally substituted with an O-protecting group; (9) nitro; (10) oxo (e.g., carboxyaldehyde or acyl); (11) C₁₋₇ spirocyclyl; (12) thioalkoxy; (13) thiol; (14) —CO₂R^{A}′, optionally substituted with an O-protecting group and where R^{A}′ is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ to aryl, (d) hydrogen, (e) C₁₋₆ alkyl-C₆₋₁₀ aryl, (f) amino-C₁₋₂₀ alkyl, (g) polyethylene glycol of —(CH₂)ₛ₂ (OCH₂CH₂)ₛ₁ (CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1} (CH₂)ₛ₂ (CH₂CH₂O)ₛ₁ (CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (15) —C(O) NR^{B}′R^{C}′, where each of R^{B}′ and R^{C}′ is, independently, selected from the group consisting of (a) hydrogen, (b) C₁₋₆ alkyl, (c) C₆₋₁₀ to aryl, and (d) C₁₋₆ alkyl-C₆₋₁₀ aryl; (16) —SO₂R^{D}′, where R^{D}′ is selected from the group consisting of (a) C₁₋₆ alkyl, (b) C₆₋₁₀ aryl, (c) C₁₋₆ alkyl-C₆₋₁₀ aryl, and (d) hydroxy; (17) —SO₂NR^{E}′R^{F}′, where each of R^{E}′ and R^{F}′ is, independently, selected from the group consisting of (a) hydrogen, (b) C₁₋₆ alkyl, (c) C₆₋₁₀ aryl and (d) C₁₋₆ alkyl-C₆₋₁₀ aryl; (18) —C(O) R^{G}′, where R^{G}′ is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alkyl-C₆₋₁₀ aryl, (f) amino-C₁₋₂₀ alkyl, (g) polyethylene glycol of —(CH₂)ₛ₂ (OCH₂CH₂)ₛ₁ (CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1} (CH₂)ₛ₂ (CH₂CH₂O)ₛ₁ (CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (19) —NRH′C(O) R′, wherein RH′ is selected from the group consisting of (a1) hydrogen and (b1) C₁₋₆ alkyl, and R″ is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alkyl-C₆₋₁₀ aryl, (f2) amino-C₁₋₂₀, alkyl, (g2) polyethylene glycol of —(CH₂)ₛ₂ (OCH₂CH₂)ₛ₁ (CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1} (CH₂)ₛ₂ (CH₂CH₂O)ₛ₁ (CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (20) —NR″C(O)OR^{K}′, wherein R′ is selected from the group consisting of (a1) hydrogen and (b1) C₁₋₆ alkyl, and R^{K}′ is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alkyl-C₆₋₁₀ aryl, (f2) amino-C₁₋₂₀ alkyl, (g2) polyethylene glycol of —(CH₂)ₛ₂ (OCH₂CH₂)ₛ₁ (CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1} (CH₂)ₛ₂ (CH₂CH₂O)ₛ₁ (CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; and (21) amidine. In some embodiments, each of these groups can be further substituted as described herein. For example, the alkyl group of a C₁-alkaryl can be further substituted with an oxo group to afford the respective aryloyl substituent.

The term “lower alkyl” means a group having one to six carbons in the chain which chain may be straight or branched. Non-limiting examples of suitable alkyl groups include methyl, ethyl, n-propyl, isopropyl, n-butyl, t-butyl, n-pentyl, and hexyl.

The term “alkylsulfinyl,” as used herein, represents an alkyl group attached to the parent molecular group through an —S(O)— group. Exemplary unsubstituted alkylsulfinyl groups are from 1 to 6, from 1 to 10, or from 1 to 20 carbons. In some embodiments, the alkyl group can be further substituted with 1, 2, 3, or 4 substituent groups as defined herein.

The term “alkylsulfinylalkyl,” as used herein, represents an alkyl group, as defined herein, substituted by an alkylsulfinyl group. Exemplary unsubstituted alkylsulfinylalkyl groups are from 2 to 12, from 2 to 20, or from 2 to 40 carbons. In some embodiments, each alkyl group can be further substituted with 1, 2, 3, or 4 substituent groups as defined herein.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, 1-propynyl, and the like. Alkynyl groups may be optionally substituted with 1, 2, 3, or 4 substituent groups that are selected, independently, from aryl, cycloalkyl, or heterocyclyl (e.g., heteroaryl), as defined herein, or any of the exemplary alkyl substituent groups described herein.

The term “amino,” as used herein, represents-N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkenyl, alkynyl, alkoxy, aryl, alkaryl, cycloalkyl, alkylcycloalkyl, carboxyalkyl (e.g., optionally substituted with an O-protecting group, such as optionally substituted arylalkoxycarbonyl groups or any described herein), sulfoalkyl, acyl (e.g., acetyl, trifluoroacetyl, or others described herein), alkoxycarbonylalkyl (e.g., optionally substituted with an O-protecting group, such as optionally substituted arylalkoxycarbonyl groups or any described herein), heterocyclyl (e.g., heteroaryl), or alkylheterocyclyl (e.g., alkylheteroaryl), wherein each of these recited R^{N1} groups can be optionally substituted, as defined herein for each group; or two R^{N1} combine to form a heterocyclyl or an N-protecting group, and wherein each R^{N2} is, independently, H, alkyl, or aryl. The amino groups of the disclosure can be an unsubstituted amino (i.e., —NH₂) or a substituted amino (i.e., —N(R′)₂). In a preferred embodiment, amino is-NH₂ or —NHR^{N1}, wherein R^{N1} is, independently, OH, NO₂, NH₂, NR^{N2} ₂, SO₂OR^{N2}, SO₂R^{N2}, SOR^{N2}, alkyl, carboxyalkyl, sulfoalkyl, acyl (e.g., acetyl, trifluoroacetyl, or others described herein), alkoxycarbonylalkyl (e.g., t-butoxycarbonylalkyl) or aryl, and each R^{N2} can be H, C₁₋₂₀ alkyl (e.g., C₁₋₆ alkyl), or C₁₋₁₀ aryl.

The term “amino acid,” as described herein, refers to a molecule having a side chain, an amino group, and an acid group (e.g., a carboxy group of —CO₂H or a sulfo group of —SO₃H), wherein the amino acid is attached to the parent molecular group by the side chain, amino group, or acid group (e.g., the side chain). In some embodiments, the amino acid is attached to the parent molecular group by a carbonyl group, where the side chain or amino group is attached to the carbonyl group. Exemplary side chains include an optionally substituted alkyl, aryl, heterocyclyl, alkylaryl, alkylheterocyclyl, aminoalkyl, carbamoylalkyl, and carboxyalkyl. Exemplary amino acids include alanine, arginine, asparagine, aspartic acid, cysteine, glutamic acid, glutamine, glycine, histidine, hydroxynorvaline, isoleucine, leucine, lysine, methionine, norvaline, ornithine, phenylalanine, proline, pyrrolysine, selenocysteine, serine, taurine, threonine, tryptophan, tyrosine, and valine. Amino acid groups may be optionally substituted with one, two, three, or, in the case of amino acid groups of two carbons or more, four substituents independently selected from the group consisting of: (1) C₁₋₆ alkoxy; (2) C₁₋₆ alkylsulfinyl; (3) amino, as defined herein (e.g., unsubstituted amino (i.e., —NH₂) or a substituted amino (i.e., —N(R^{N1})₂, where R^{N1} is as defined for amino); (4) C₆₋₁₀ aryl-C₁₋₆ alkoxy; (5) azido; (6) halo; (7) (C₂₋₉ heterocyclyl)oxy; (8) hydroxy; (9) nitro; (10) oxo (e.g., carboxyaldehyde or acyl); (11) C₁₋₇ spirocyclyl; (12) thioalkoxy; (13) thiol; (14) —CO₂R^{A}′, where R^{A}′ is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alkyl-C₆₋₁₀ aryl, (f) amino-C₁₋₂₀ alkyl, (g) polyethylene glycol of —(CH₂)ₛ₂ (OCH₂CH₂)ₛ₁ (CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1} (CH₂)ₛ₂ (CH₂CH₂O)ₛ₁ (CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (15) —C(O) NRBRC′, where each of R^{B}′ and R^{C}′ is, independently, selected from the group consisting of (a) hydrogen, (b) C₁₋₆ alkyl, (c) C₆₋₁₀ aryl, and (d) C₁₋₆ alkyl-C₆₋₁₀ aryl; (16) —SO₂R^{D}′, where R^{D}′ is selected from the group consisting of (a) C₁₋₆ alkyl, (b) C₆₋₁₀ aryl, (c) C₁₋₆ alkyl-C₆₋₁₀ aryl, and (d) hydroxy; (17) —SO₂NR^{E}′R^{F}′, where each of R^{E}′ and R^{F}′ is, independently, selected from the group consisting of (a) hydrogen, (b) C₁₋₆ alkyl, (c) C₆₋₁₀ aryl and (d) C₁₋₆ alkyl-C₆₋₁₀ aryl; (18) —C(O) R^{G}′, where R^{G}′ is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alkyl-C₆₋₁₀ aryl, (f) amino-C₁₋₂₀ alkyl, (g) polyethylene glycol of —(CH₂)ₛ₂ (OCH₂CH₂)ₛ₁ (CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1} (CH₂)ₛ₂ (CH₂CH₂O)ₛ₁ (CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each RNl is, independently, hydrogen or optionally substituted C₁₋₆ alkyl; (19) —NRH′C(O) R′, wherein RH′ is selected from the group consisting of (a1) hydrogen and (b1) C₁₋₆ alkyl, and R″ is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alkyl-C₆₋₁₀ aryl, (f2) amino-C₁₋₂₀ alkyl, (g2) polyethylene glycol of —(CH₂)ₛ₂ (OCH₂CH₂)ₛ₁ (CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1} (CH₂)ₛ₂ (CH₂CH₂O)ₛ₁ (CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (20) —NR^{J}′C(O)OR^{K}′, wherein R^{J}′ is selected from the group consisting of (a1) hydrogen and (b1) C₁₋₆ alkyl, and R^{K}′ is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alkyl-C₆₋₁₀ aryl, (f2) amino-C₁₋₂₀ alkyl, (g2) polyethylene glycol of —(CH₂)ₛ₂ (OCH₂CH₂)ₛ₁ (CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1} (CH)ₛ₂ (CH₂CH₂O)ₛ₁ (CH₂)ₛ₃NR^{N1} wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; and amidine. In some embodiments, each of these groups can be further substituted as described herein.

The term “aminoalkoxy,” as used herein, represents an alkoxy group, as defined herein, substituted by an amino group, as defined herein. The alkyl and amino each can be further substituted with 1, 2, 3, or 4 substituent groups as described herein for the respective group (e.g., CO₂R^{A}′, where R^{A}′ is selected from the group consisting of (a) C₁₋₆ alkyl, (b) C₆₋₁₀ aryl, (c) hydrogen, and (d) C₁₋₆ alkyl-C₆₋₁₀ aryl, e.g., carboxy).

The term “aminoalkyl,” as used herein, represents an alkyl group, as defined herein, substituted by an amino group, as defined herein. The alkyl and amino each can be further substituted with 1, 2, 3, or 4 substituent groups as described herein for the respective group (e.g., CO₂R^{A}′, where R^{A}′ is selected from the group consisting of (a) C₁₋₆ alkyl, (b) C₆₋₁₀ aryl, (c) hydrogen, and (d) C₁₋₆ alkyl-C₆₋₁₀ aryl, e.g., carboxy, and/or an N-protecting group).

The term “aminoalkenyl,” as used herein, represents an alkenyl group, as defined herein, substituted by an amino group, as defined herein. The alkenyl and amino each can be further substituted with 1, 2, 3, or 4 substituent groups as described herein for the respective group (e.g., CO₂R^{A}′, where R^{A}′ is selected from the group consisting of (a) C₁₋₆ alkyl, (b) C₆₋₁₀ aryl, (c) hydrogen, and (d) C₁₋₆ alkyl-C₆₋₁₀ aryl, e.g., carboxy, and/or an N-protecting group).

The term “anionic lipid” means a lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

The phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The terms “include,” “have,” or the like is used in the description or the claims, such term is intended to be inclusive in a manner similar to the term “comprise” as “comprise” is interpreted when employed as a transitional word in a claim.

The term “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

The term “boranyl,” as used herein, represents —B(R^{B1})₃, where each R^{B1} is, independently, selected from the group consisting of H and optionally substituted alkyl. In some embodiments, the boranyl group can be substituted with 1, 2, 3, or 4 substituents as defined herein for alkyl.

The term “biocompatible” means compatible with living cells, tissues, organs or systems posing little to no risk of injury, toxicity or rejection by the immune system.

The term “biodegradable” means capable of being broken down into innocuous products by the action of living things.

The phrase “biologically active” refers to a characteristic of any substance that has activity in a biological system and/or organism. For instance, a substance that, when administered to an organism, has a biological effect on that organism, is considered to be biologically active. In particular embodiments, a polynucleotide of the present disclosure may be considered biologically active if even a portion of the polynucleotide is biologically active or mimics an activity considered biologically relevant.

The term “boranophosphate” has the ordinary meaning as understood in the art and can include protonated, deprotonated, and tautomeric forms thereof. For example, a

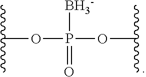

boranophosphate within the context of a compound can have the structure

The terms “carbocyclic” and “carbocyclyl,” as used herein, refer to an optionally substituted C₃₋₁₂ monocyclic, bicyclic, or tricyclic structure in which the rings, which may be aromatic or non-aromatic, are formed by carbon atoms. Carbocyclic structures include cycloalkyl, cycloalkenyl, and aryl groups.

The term “carbamoyl,” as used herein, represents-C(O)—N(R^{N1})₂, where the meaning of each R^{N1} is found in the definition of “amino” provided herein.

The term “carbamoylalkyl,” as used herein, represents an alkyl group, as defined herein, substituted by a carbamoyl group, as defined herein. The alkyl group can be further substituted with 1, 2, 3, or 4 substituent groups as described herein.

The term “carbamyl,” as used herein, refers to a carbamate group having the structure-NR^{N1}C(═O) OR or —OC(═O) N(R^{N1})₂, where the meaning of each R^{N1} is found in the definition of “amino” provided herein, and R is alkyl, cycloalkyl, alkylcycloalkyl, aryl, alkylaryl, heterocyclyl (e.g., heteroaryl), or alkylheterocyclyl (e.g., alkylheteroaryl), as defined herein.

The term “carbonyl,” as used herein, represents a C(O) group, which can also be represented as C═O.

The term “carboxyaldehyde” represents an acyl group having the structure-C(O) H.

The term “carboxy,” as used herein, means-CO₂H.

The term “cationic lipid” means amphiphilic lipids and salts thereof having a positive, hydrophilic head group; one, two, three, or more hydrophobic fatty acid or fatty alkyl chains; and a connector between these two domains. An ionizable or protonatable cationic lipid is typically protonated (i.e., positively charged) at a pH below its pKa and is substantially neutral at a pH above the pKa. Preferred ionizable cationic lipids are those having a pKa that is less than physiological pH, which is typically about 7.4. The cationic lipids of the disclosure may also be termed titratable cationic lipids. The cationic lipids can be an “amino lipid” having a protonatable tertiary amine (e.g., pH-titratable) head group. Some amino exemplary amino lipid can include C₁₈ alkyl chains, wherein each alkyl chain independently has 0 to 3 (e.g., 0, 1, 2, or 3) double bonds; and ether, ester, or ketal linkages between the head group and alkyl chains. Such cationic lipids include, but are not limited to, DSDMA, DODMA, DLinDMA, DLenDMA, Y-DLenDMA, DLin-K-DMA, DLin-K—C₂-DMA (also known as DLin-C₂K-DMA, XTC2, and C₂K), DLin-K—C₃-DM A, DLin-K—C₄-DMA, DLen-C₂K-DMA, y-DLen-C₂K-DMA, DLin-M-C₂-DMA (also known as MC2), DLin-M-C₃-DMA (also known as MC3) and (DLin-MP-DMA) (also known as 1-B1 1).

The term “comprising” is intended to be open and permits but does not require the inclusion of additional elements or steps. When the term “comprising” is used herein, the term “consisting of” is thus also encompassed and disclosed.

The term “composition” means a product comprising the specified ingredients in the specified amounts, as well as any product that results, directly or indirectly, from combination of the specified ingredients in the specified amounts.

The term “commercially available chemicals” and the chemicals used in the Examples set forth herein may be obtained from standard commercial sources, where such sources include, for example, Acros Organics (Pittsburgh, Pa.), Sigma-Adrich Chemical (Milwaukee, Wis.), Avocado Research (Lancashire, U.K.), Bionet (Cornwall, U.K.), Boron Molecular (Research Triangle Park, N.C.), Combi-Blocks (San Diego, Calif.), Eastman Organic Chemicals, Eastman Kodak Company (Rochester, N.Y.), Fisher Scientific Co. (Pittsburgh, Pa.), Frontier Scientific (Logan, Utah), ICN Biomedicals, Inc. (Costa Mesa, Calif.), Lancaster Synthesis (Windham, N. H.), Maybridge Chemical Co. (Cornwall, U.K.), Pierce Chemical Co. (Rockford, Ill.), Riedel de Haen (Hannover, Germany), Spectrum Quality Product, Inc. (New Brunswick, N.J.), TCI America (Portland, Oreg.), and Wako Chemicals USA, Inc. (Richmond, Va.).

The phrase “compounds described in the chemical literature” may be identified through reference books and databases directed to chemical compounds and chemical reactions, as known to one of ordinary skill in the art. Suitable reference books and treatise that detail the synthesis of reactants useful in the preparation of compounds disclosed herein, or provide references to articles that describe the preparation of compounds disclosed herein, include for example, “Synthetic Organic Chemistry”, John Wiley and Sons, Inc. New York; S. R. Sandler et al, “Organic Functional Group Preparations,” 2nd Ed., Academic Press, New York, 1983; H. O. House, “Modern Synthetic Reactions,” 2nd Ed., W. A. Benjamin, Inc. Menlo Park, Calif., 1972; T. L. Glichrist, “Heterocyclic Chemistry,” 2nd Ed. John Wiley and Sons, New York, 1992; J. March, “Advanced Organic Chemistry: reactions, Mechanisms and Structure,” 5th Ed., Wiley Interscience, New York, 2001; Specific and analogous reactants may also be identified through the indices of known chemicals prepared by the Chemical Abstract Service of the American Chemical Society, which are available in most public and university libraries, as well as through online databases (the American Chemical Society, Washington, D.C. may be contacted for more details). Chemicals that are known but not commercially available in catalogs may be prepared by custom chemical synthesis houses, where many of the standard chemical supply houses (such as those listed above) provide custom synthesis services.

The term “complementary nucleotide bases” means a pair of nucleotide bases that form hydrogen bonds with each other. Adenine (A) pairs with thymine (T) in DNA or with uracil (U) in RNA, and guanine (G) pairs with cytosine (C). Complementary segments or strands of nucleic acid hybridize (i.e. join by hydrogen bonding) with each other. By “complementary” is meant that a nucleic acid can form hydrogen bond(s) with another nucleic acid sequence either by traditional Watson-Crick or by other non-traditional modes of binding.

The term “cycloalkyl,” as used herein represents a monovalent saturated or unsaturated non-aromatic cyclic hydrocarbon group from three to eight carbons, unless otherwise specified, and is exemplified by cyclopropyl, cyclobutyl, cyclopentyl, cyclohexyl, cycloheptyl, bicycle heptyl, and the like. When the cycloalkyl group includes one carbon-carbon double bond, the cycloalkyl group can be referred to as a “cycloalkenyl” group. Exemplary cycloalkenyl groups include cyclopentenyl, cyclohexenyl, and the like. The cycloalkyl groups of this disclosure can be optionally substituted with: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁₋₆ thioalkoxy-C₁₋₆ alkyl); (3) C₁₂ alkoxy (e.g., C₁₋₆ alkoxy, such as perfluoroalkoxy); (4) C₁₋₆ alkylsulfinyl; (5) C₆₋₁₀ aryl; (6) amino; (7) C₁₋₆ alkyl-C₆₋₁₀ aryl; (8) azido; (9) C₃-8 cycloalkyl; (10) C₁₋₆ alkyl-C₃-8 cycloalkyl; (11) halo; (12) C₁₋₁₂ heterocyclyl (e.g., C₁₋₁₂ heteroaryl); (13) (C₁₋₁₂ heterocyclyl)oxy; (14) hydroxy; (15) nitro; (16) C₁₋₂₀ thioalkoxy (e.g., C₁₋₆ thioalkoxy); (17) —(CH₂)_{q}CO₂R^{A}′, where q is an integer from zero to four, and R^{A}′ is selected from the group consisting of (a) C₁₋₆ alkyl, (b) C₆₋₁₀ aryl, (c) hydrogen, and (d) C₁₋₆ alkyl-C₆₋₁₀ aryl; (18) —(CH₂)_{q}CONR^{B}′R^{C}′, where q is an integer from zero to four and where R^{B}′ and R^{C}′ are independently selected from the group consisting of (a) hydrogen, (b) C₆₋₁₀ alkyl, (c) C₆₋₁₀ aryl, and (d) C₁₋₆ alkyl-C₆₋₁₀ aryl; (19) —(CH₂)_{q}SO₂R^{D}′, where q is an integer from zero to four and where R^{D}′ is selected from the group consisting of (a) C₆₋₁₀ alkyl, (b) C₆₋₁₀ aryl, and (c) C₁₋₆ alkyl-C₆₋₁₀ aryl; (20) —(CH₂)_{q}SO₂NR^{E}′R^{F}′, where q is an integer from zero to four and where each of R^{E}′ and R^{F}′ is, independently, selected from the group consisting of (a) hydrogen, (b) C₆₋₁₀ alkyl, (c) C₆₋₁₀ aryl, and (d) C₁₋₆ alkyl-C₁₋₁₀ aryl; (21) thiol; (22) C₆₋₁₀ aryloxy; (23) C₃-8 cycloalkoxy; (24) C₆₋₁₀ aryl-C₁₋₆ alkoxy; (25) C₁₋₆ alkyl-C₁₋₁₂ heterocyclyl (e.g., C₁₋₆ alkyl-C₁₋₁₂ heteroaryl); (26) oxo; (27) C₂₋₂₀ alkenyl; and (28) C₂₋₂₀ alkynyl. In some embodiments, each of these groups can be further substituted as described herein. For example, the alkyl group of a C₁-alkaryl or a C₁-alkylheterocyclyl can be further substituted with an oxo group to afford the respective aryloyl and (heterocyclyl) oyl substituent group.

The term “diastereomer,” as used herein means stereoisomers that are not mirror images of one another and are non-superimposable on one another.

The term “diacylglycerol” or “DAG” includes a compound having 2 fatty acyl chains, R¹ and R², both of which have independently between 2 and 30 carbons bonded to the 1- and 2-position of glycerol by ester linkages. The acyl groups can be saturated or have varying degrees of unsaturation. Suitable acyl groups include, but are not limited to, lauroyl (C₁₂), myristoyl (C₁₄), palmitoyl (C₁₆), stearoyl (C₁₈), and icosoyl (C₂₀). In preferred embodiments, R¹ and R² are the same, i.e., R¹ and R² are both myristoyl (i.e., dimyristoyl), R¹ and R² are both stearoyl (i.e., distearoyl).

The term “dialkyloxypropyl” or “DAA” includes a compound having 2 alkyl chains, R and R, both of which have independently between 2 and 30 carbons. The alkyl groups can be saturated or have varying degrees of unsaturation.

The term “effective amount” of an agent, as used herein, is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

The term “enantiomer,” as used herein, means each individual optically active form of a compound of the disclosure, having an optical purity or enantiomeric excess (as determined by methods standard in the art) of at least 80% (i.e., at least 90% of one enantiomer and at most 10% of the other enantiomer), preferably at least 90% and more preferably at least 98%.

An “enzyme having ornithine transcarbamylase activity”, an “enzyme having OTC activity” or “an OTC enzyme” means a protein or an enzyme that catalyzes a reaction between carbamoyl phosphate and ornithine to form citrulline and phosphate. OTC plays an essential role in the urea cycle which has the purpose of capturing toxic ammonia and transforming it into a less toxic urea nitrogen source for excretion.

The term “fully encapsulated” means that the nucleic acid (e.g., mRNA) in the nucleic acid-lipid particle is not significantly degraded after exposure to serum or a nuclease assay that would significantly degrade free RNA. When fully encapsulated, preferably less than 25% of the nucleic acid in the particle is degraded in a treatment that would normally degrade 100% of free nucleic acid, more preferably less than 10%, and most preferably less than 5% of the nucleic acid in the particle is degraded. “Fully encapsulated” also means that the nucleic acid-lipid particles do not rapidly decompose into their component parts upon in vivo administration.

The terms “halo” and “Halogen”, as used herein, represents a halogen selected from bromine, chlorine, iodine, or fluorine.

The term “haloalkyl,” as used herein, represents an alkyl group, as defined herein, substituted by a halogen group (i.e., F, Cl, Br, or I). A haloalkyl may be substituted with one, two, three, or, in the case of alkyl groups of two carbons or more, four halogens. Haloalkyl groups include perfluoroalkyls (e.g., —CF₃), —CHF₂, —CH₂F, —CCl₃, —CH₂CH₂Br, —CH₂CH(CH₂CH₂Br) CH₃, and —CHICH₃. In some embodiments, the haloalkyl group can be further substituted with 1, 2, 3, or 4 substituent groups as described herein for alkyl groups.

The term “heteroalkyl,” as used herein, refers to an alkyl group, as defined herein, in which one or two of the constituent carbon atoms have each been replaced by nitrogen, oxygen, or sulfur. In some embodiments, the heteroalkyl group can be further substituted with 1, 2, 3, or 4 substituent groups as described herein for alkyl groups.

The term “hydrocarbon,” as used herein, represents a group consisting only of carbon and hydrogen atoms.

The term “hydroxy,” as used herein, represents an —OH group. In some embodiments, the hydroxy group can be substituted with 1, 2, 3, or 4 substituent groups (e.g., O-protecting groups) as defined herein for an alkyl.

The term “hydroxyalkenyl,” as used herein, represents an alkenyl group, as defined herein, substituted by one to three hydroxy groups, with the proviso that no more than one hydroxy group may be attached to a single carbon atom of the alkyl group, and is exemplified by dihydroxypropenyl, hydroxyisopentenyl, and the like. In some embodiments, the hydroxyalkenyl group can be substituted with 1, 2, 3, or 4 substituent groups (e.g., O-protecting groups) as defined herein for an alkyl.

The term “hydroxyalkyl,” as used herein, represents an alkyl group, as defined herein, substituted by one to three hydroxy groups, with the proviso that no more than one hydroxy group may be attached to a single carbon atom of the alkyl group, and is exemplified by hydroxymethyl, dihydroxypropyl, and the like. In some embodiments, the hydroxyalkyl group can be substituted with 1, 2, 3, or 4 substituent groups (e.g., O-protecting groups) as defined herein for an alkyl.

The term “hydrate” means a solvate wherein the solvent molecule is H₂O.

The term “isomer,” as used herein, means any tautomer, stereoisomer, enantiomer, or diastereomer of any compound of the disclosure. It is recognized that the compounds of the disclosure can have one or more chiral centers and/or double bonds and, therefore, exist as stereoisomers, such as double-bond isomers (i.e., geometric E/Z isomers) or diastereomers (e.g., enantiomers (i.e., (+) or (−)) or cis/trans isomers). According to the disclosure, the chemical structures depicted herein, and therefore the compounds of the disclosure, encompass all of the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the disclosure can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

The term “nucleic acid” means deoxyribonucleotides or ribonucleotides and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2′-O-methyl ribonucleotides, peptide-nucleic acids (PNAs).

The term “oxo” as used herein, represents=O.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers and/or conformers of the basic molecular structure. Some compounds of the present disclosure may exist in different tautomeric forms, all of the latter being included within the scope of the present disclosure.

The term “sulfonyl,” as used herein, represents an —S(O)₂— group.

The term “compound,” is meant to include all stereoisomers, geometric isomers, tautomers, and isotopes of the structures depicted.

The term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences. In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an oligonucleotide or polypeptide or may apply to a portion, region or feature thereof.

The term “cyclic” refers to the presence of a continuous loop. Cyclic molecules need not be circular, only joined to form an unbroken chain of subunits. Cyclic molecules such as the mRNA of the present disclosure may be single units or multimers or comprise one or more components of a complex or higher order structure.

The term “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

The term “delivery” refers to the act or manner of delivering a compound, substance, entity, moiety, cargo or payload.

The term “delivery agent” or “delivery vehicle” refers to any substance which facilitates, at least in part, the in vivo delivery of a polynucleotide to targeted cells.

The term “digest” means to break apart into smaller pieces or components. When referring to polypeptides or proteins, digestion results in the production of peptides.

The phrase “encoded protein cleavage signal” refers to the nucleotide sequence which encodes a protein cleavage signal.

The term “engineered” when they are designed to have a feature or property, whether structural or chemical, that varies from a starting point, wild type or native molecule.

The term “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

The term “feature” refers to a characteristic, a property, or a distinctive element.

The term “fragment,” as used herein, refers to a portion. For example, fragments of proteins may comprise polypeptides obtained by digesting full-length protein isolated from cultured cells.

The term “functional” biological molecule is a biological molecule in a form in which it exhibits a property and/or activity by which it is characterized.

The term “homology” refers to the overall relatedness between polymeric molecules, e.g. between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids.

The term “hydrophobic lipids” means compounds having apolar groups that include, but are not limited to, long-chain saturated and unsaturated aliphatic hydrocarbon groups and such groups optionally substituted by one or more aromatic, cycloaliphatic, or heterocyclic group(s). Suitable examples include, but are not limited to, diacylglycerol, dialkylglycerol, N—N-dialkylamino, 1,2-diacyloxy-3-aminopropane, and 1,2-dialkyl-3-aminopropane.

The term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12 (1), 387 (1984)), BLASTP, BLASTN, and FASTA Altschul, S. F. et al., J. Molec. Biol., 215, 403 (1990)).

The term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

The term “lipid” means an organic compound that comprises an ester of fatty acid and is characterized by being insoluble in water, but soluble in many organic solvents. Lipids are usually divided into at least three classes: (1) “simple lipids,” which include fats and oils as well as waxes; (2) “compound lipids,” which include phospholipids and glycolipids; and (3) “derived lipids” such as steroids.

The term “lipid delivery vehicle” means a lipid formulation that can be used to deliver a therapeutic nucleic acid (e.g., mRNA) to a target site of interest (e.g., cell, tissue, organ, and the like). The lipid delivery vehicle can be a nucleic acid-lipid particle, which can be formed from a cationic lipid, a non-cationic lipid (e.g., a phospholipid), a conjugated lipid that prevents aggregation of the particle (e.g., a PEG-lipid), and optionally cholesterol. Typically, the therapeutic nucleic acid (e.g., mRNA) may be encapsulated in the lipid portion of the particle, thereby protecting it from enzymatic degradation.

The term “lipid encapsulated” means a nucleic acid such as an mRNA that is completely encapsulated, partial encapsulated, or both in a lipid formulation. In a preferred embodiment, the nucleic acid (e.g., mRNA) is fully encapsulated in the lipid particle.

The term “lipid conjugate” means a conjugated lipid that inhibits aggregation of lipid particles. Such lipid conjugates include, but are not limited to, PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides, cationic PEG lipids, polyoxazoline (POZ)-lipid conjugates, polyamide oligomers, and mixtures thereof. PEG or POZ can be conjugated directly to the lipid or may be linked to the lipid via a linker moiety. Any linker moiety suitable for coupling the PEG or the POZ to a lipid can be used including, e.g., non-ester-containing linker moieties and ester-containing linker moieties. In certain preferred embodiments, non-ester-containing linker moieties, such as amides or carbamates, are used.

The term “amphipathic lipid” or “amphiphilic lipid” means the material in which the hydrophobic portion of the lipid material orients into a hydrophobic phase, while the hydrophilic portion orients toward the aqueous phase. Hydrophilic characteristics derive from the presence of polar or charged groups such as carbohydrates, phosphate, carboxylic, sulfato, amino, sulfhydryl, nitro, hydroxyl, and other like groups. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). Examples of amphipathic compounds include, but are not limited to, phospholipids, aminolipids, and sphingolipids.

The term “linker” refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker may be of sufficient length as to not interfere with incorporation into a nucleic acid sequence. The linker can be used for any useful purpose, such as to form multimers (e.g., through linkage of two or more polynucleotides) or conjugates, as well as to administer a payload, as described herein. Examples of chemical groups that can be incorporated into the linker include, but are not limited to, alkyl, alkenyl, alkynyl, amido, amino, ether, thioether, ester, alkyl, heteroalkyl, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers, Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris (2-carboxyethyl) phosphine (TCEP), or other reducing agents, and/or photolysis, as well as an ester bond can be cleaved for example by acidic or basic hydrolysis.

The term “mammal” means a human or other mammal or means a human being.

The term “messenger RNA” (mRNA) refers to any polynucleotide which encodes a protein or polypeptide of interest and which is capable of being translated to produce the encoded protein or polypeptide of interest in vitro, in vivo, in situ or ex vivo.

The term “modified” refers to a changed state or structure of a molecule of the disclosure or of an otherwise standard reference molecule. Molecules may be modified in many ways including chemically, structurally, and functionally. In one embodiment, the mRNA molecules of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they may differ from the chemical structure of the A, C, G, U ribonucleotides.

The term “naturally occurring” means existing in nature without artificial aid.

The term “nonhuman vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

The term “nucleotide” is meant to include nucleotides that have natural bases (standard) or modified bases well known in the art. Such bases are generally located at the 1′ position of a nucleotide sugar moiety. Nucleotides generally comprise a base, sugar, and a phosphate group. The nucleotides can be unmodified or modified at the sugar, phosphate, and/or base moiety, (also referred to interchangeably as nucleotide analogs, modified nucleotides, non-natural nucleotides, non-standard nucleotides and other; see, for example, Usman and McSwiggen, supra; Eckstein, et al., International PCT Publication No. WO 92/07065; Usman, et al., International PCT Publication No. WO 93/15187; Uhlman & Peyman, supra, all are hereby incorporated by reference herein). There are several examples of modified nucleic acid bases known in the art as summarized by Limbach, et al, Nucleic Acids Res. 22:2183, 1994. Some of the non-limiting examples of base modifications that can be introduced into nucleic acid molecules include: inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g., 6-methyluridine), propyne, and others (Burgin, et al., Biochemistry 35:14090, 1996; Uhlman & Peyman, supra). By “modified bases” in this aspect is meant nucleotide bases other than adenine, guanine, cytosine, and uracil at 1′ position or their equivalents.

The term “off target” refers to any unintended effect on any one or more target, gene, or cellular transcript.

The term “codon-optimized” means a natural (or purposefully designed variant of a natural) coding sequence which has been redesigned by choosing different codons without altering the encoded protein amino acid sequence increasing the protein expression levels (Gustafsson et al, Codon bias and heterologous protein expression. 2004, Trends Biotechnol 22:346-53). Variables such as high codon adaptation index (CAI), LowU method, mRNA secondary structures, cis-regulatory sequences, GC content and many other similar variables have been shown to somewhat correlate with protein expression levels (Villalobos et al., Gene Designer: a synthetic biology tool for constructing artificial DNA segments. 2006, BMC Bioinformatics 7:285). High CAI (codon adaptation index) method picks a most frequently used synonymous codon for an entire protein coding sequence. The most frequently used codon for each amino acid is deduced from 74218 protein-coding genes from a human genome. The LowU method targets only Li-containing codons that can be replaced with a synonymous codon with fewer U moieties. If there are a few choices for the replacement, the more frequently used codon will be selected. The remaining codons in the sequence are not changed by the LowU method. This method may be used in conjunction with the disclosed mRNAs to design coding sequences that are to be synthesized with 5-methoxy uridine.

The term “open reading frame” or “ORF” to a nucleic acid sequence (DNA or RNA) refers to the portion of a sequence that is capable of encoding a polypeptide of interest. ORFs generally begin with the start codon ATG, and end with a nonsense or termination codon or signal.

The phrase “operably linked” refers to a functional connection between two or more molecules, constructs, transcripts, entities, moieties or the like.

The term “patient” refers to a subject who may seek or be in need of treatment, requires treatment, is receiving treatment, will receive treatment, or a subject who is under care by a trained professional for a particular disease or condition.

The phrase “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g. alkyl) per se is optional.

The term “peptide” is less than or equal to 50 amino acids long, e.g., about 5, 10, 15, 20, 25, 30, 35, 40, 45, or 50 amino acids long.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

The phrase “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

The term “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

The term “pharmaceutically acceptable solvate,” as used herein, means a compound of the disclosure wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “phosphate” is used in its ordinary sense as understood by those skilled in the art and includes its protonated forms, for example

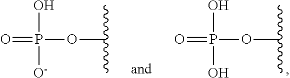 [SMILES: *OP(=O)([O-])O ; *OP(=O)(O)O]

As used herein, the terms “monophosphate,” “diphosphate,” and “triphosphate” are used in their ordinary sense as understood by those skilled in the art, and include protonated forms.

The term “phosphorothioate” refers to a compound of the general formula

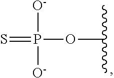

its protonated forms, for example,

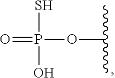 [SMILES: *OP(=O)(O)S]

and its tautomers such as

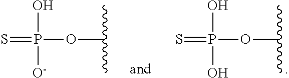 [SMILES: *OP([O-])(O)=S ; *OP(O)(O)=S]

The term “physicochemical” means of or relating to a physical and/or chemical property.

The term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

The term “protein cleavage site” refers to a site where controlled cleavage of the amino acid chain can be accomplished by chemical, enzymatic or photochemical means.

The phrase “protein cleavage signal” refers to at least one amino acid that flags or marks a polypeptide for cleavage.

The term “proteins of interest” or “desired proteins” include those provided herein and fragments, mutants, variants, and alterations thereof.

The terms “purify,” “purified,” “purification” means to make substantially pure or clear from unwanted components, material defilement, admixture or imperfection.

The term “RNA” means a molecule comprising at least one ribonucleotide residue. By “ribonucleotide” is meant a nucleotide with a hydroxyl group at the 2′ position of a β-D-ribo-furanose moiety. The terms includes double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution, and/or alteration of one or more nucleotides. Such alterations can include addition of non-nucleotide material, such as to the end(s) of an interfering RNA or internally, for example at one or more nucleotides of the RNA. Nucleotides in the RNA molecules of the instant disclosure can also comprise non-standard nucleotides, such as non-naturally occurring nucleotides or chemically synthesized nucleotides or deoxynucleotides. These altered RNAs can be referred to as analogs or analogs of naturally-occurring RNA. As used herein, the terms “ribonucleic acid” and “RNA” refer to a molecule containing at least one ribonucleotide residue, including siRNA, antisense RNA, single stranded RNA, microRNA, mRNA, noncoding RNA, and multivalent RNA.

The term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

The phrases “signal sequences” or “signal peptide” refer to a sequence which can direct the transport or localization of a protein.

The terms “significant” or “significantly” are used synonymously with the term “substantially.”

The phrase “single unit dose” is a dose of any therapeutic administered in one dose/at one time/single route/single point of contact, i.e., single administration event.

The term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

The term “solvate” means a physical association of a compound of this disclosure with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like.

The term “split dose” is the division of single unit dose or total daily dose into two or more doses.

The term “stable” refers to a compound that is sufficiently robust to survive isolation to a useful degree of purity from a reaction mixture, and preferably capable of formulation into an efficacious therapeutic agent.

The terms “stabilize”, “stabilized,” “stabilized region” means to make or become stable.

The term “substituted” means substitution with specified groups other than hydrogen, or with one or more groups, moieties, or radicals which can be the same or different, with each, for example, being independently selected.

The term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The phrase “Substantially equal” relates to time differences between doses, the term means plus/minus 2%.

The phrase “substantially simultaneously” relates to plurality of doses, the term means within 2 seconds.

The phrase “suffering from” relates to an individual who is “suffering from” a disease, disorder, and/or condition has been diagnosed with or displays one or more symptoms of a disease, disorder, and/or condition.

The phrase “susceptible to” relates to an individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

The term “synthetic” means produced, prepared, and/or manufactured by the hand of man. Synthesis of polynucleotides or polypeptides or other molecules of the present disclosure may be chemical or enzymatic.

The term “targeted cells” refers to any one or more cells of interest. The cells may be found in vitro, in vivo, in situ or in the tissue or organ of an organism. The organism may be an animal, preferably a mammal, more preferably a human and most preferably a patient.

The term “therapeutic agent” refers to any agent that, when administered to a subject, has a therapeutic, diagnostic, and/or prophylactic effect and/or elicits a desired biological and/or pharmacological effect.

The term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

The term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

The term “total daily dose” is an amount given or prescribed in 24 hr period. It may be administered as a single unit dose.

The term “transcription factor” refers to a DNA-binding protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors may regulate transcription of a target gene alone or in a complex with other molecules.

The term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

The term “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H—, 2H—and 4H-1,2,4-triazole, 1H- and 2H-isoindole, and 1H- and 2H-pyrazole. Tautomeric forms can be in equilibrium or sterically locked into one form by appropriate substitution.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

The term “half-life” is the time required for a quantity such as nucleic acid or protein concentration or activity to fall to half of its value as measured at the beginning of a time period.

The term “in vitro” refers to events that occur in an artificial environment, e.g., in a test tube or reaction vessel, in cell culture, in a Petri dish, etc., rather than within an organism (e.g., animal, plant, or microbe).

The term “in vivo” refers to events that occur within an organism (e.g., animal, plant, or microbe or cell or tissue thereof).

The term “monomer” refers to a single unit, e.g., a single nucleic acid, which may be joined with another molecule of the same or different type to form an oligomer. In some embodiments, a monomer may be an unlocked nucleic acid, i.e., a UNA monomer.

The term “neutral lipid” means a lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” means an amphipathic lipid, a neutral lipid or anionic lipid as described herein.

The term “oligomer” may be used interchangeably with “polynucleotide” and refers to a molecule comprising at least two monomers and includes oligonucleotides such as DNAs and RNAs. In the case of oligomers containing RNA monomers and/or unlocked nucleic acid (UNA) monomers, the oligomers of the present disclosure may contain sequences in addition to the coding sequence (CDS). These additional sequences may be untranslated sequences, i.e., sequences which are not converted to protein by a host cell. These untranslated sequences can include a 5′ cap, a 5′ untranslated region (5′ UTR), a 3′ untranslated region (3′ UTR), and a tail region, e.g., a polyA tail region. As described in further detail herein, any of these untranslated sequences may contain one or more UNA monomers—these UNA monomers are not capable of being translated by a host cell's machinery. In the context of the present disclosure, a “mRNA sequence”, a “mRNA sequence”, “translatable polynucleotide”, or “translatable compound” refers to a sequence that comprises a region, e.g., the coding region of an RNA (e.g., the coding sequence of human CFTR or a codon-optimized version thereof), that is capable of being converted to a protein or a fragment thereof, e.g., the human CFTR protein or a fragment thereof.

The terms “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

The term “translatable” may be used interchangeably with the term “expressible” and refers to the ability of polynucleotide, or a portion thereof, to be converted to a polypeptide by a host cell. As is understood in the art, translation is the process in which ribosomes in a cell's cytoplasm create polypeptides. In translation, messenger RNA (mRNA) is decoded by tRNAs in a ribosome complex to produce a specific amino acid chain, or polypeptide. Furthermore, the term “translatable” when used in this specification in reference to an oligomer, means that at least a portion of the oligomer, e.g., the coding region of an oligomer sequence (also known as the coding sequence or CDS), is capable of being converted to a protein or a fragment thereof.

The term “translation efficiency” refers to a measure of the production of a protein or polypeptide by translation of an mRNA sequence in vitro or in vivo. This disclosure provides a range of mRNA sequence molecules wherein the mRNA sequence can be expressible to provide a polypeptide or protein.

The term “unit dose” refers to a discrete amount of the pharmaceutical composition comprising a predetermined amount of the active ingredient. The amount of the active ingredient may generally be equal to the dosage of the active ingredient which would be administered to a subject and/or a convenient fraction of such a dosage including, but not limited to, one-half or one-third of such a dosage.

### EXAMPLES

Additional embodiments of the present disclosure are illustrated in further detail in the following examples, which are not in any way intended to limit the scope of the claims.

### Example 1: In Vitro Transcription Protocol

### Material and Methods

Constructs of mRNAs described herein were synthesized in vitro using T7RNA polymerase-mediated, DNA-dependent RNA transcription. In the transcription reaction, modified and unmodified uridine triphosphates (UTP) were used depending on the desired polynucleotide configuration. Modified UTPs used included 5-methoxy UTP (5MeOU), N¹-methyl pseudo UTP, N1-methoxy methyl pseudo UTP (N1-MOM), 5-hydroxy methyl UTP, 5-carboxy UTP, and a mixture of modified UTPs, using a linearized template for each UTR combination. The mRNA was purified using column chromatography, the DNA and double stranded RNA contamination of all mRNAs was removed using an enzymatic reaction, and the mRNA was concentrated, and buffer exchanged.

### Preparation of Lipid Encapsulated mRNA

Lipid encapsulated mRNA particles were prepared by mixing lipids (ionizable cationic lipid: DSPC: Cholesterol: PEG-DMG) in ethanol with OTC mRNA dissolved in citrate buffer. The mixed material was instantaneously diluted with Phosphate Buffer. Ethanol was removed by dialysis against phosphate buffer using regenerated cellulose membrane (100 kD MWCO) or by tangential flow filtration (TFF) using modified polyethersulfone (mPES) hollow fiber membranes (100 kD MWCO). Once the ethanol was completely removed, the buffer was exchanged with HEPES (4-(2-hydroxyethyl)-1-piperazineethanesulfonic acid) buffer containing 40-60 mM NaCl and 7-12% sucrose, pH 7.3. The formulation was concentrated followed by 0.2 μm filtration using PES filters. The mRNA concentration in the formulation was then measured by Ribogreen fluorimetric assay following which the concentration was adjusted to a final desired concentration by diluting with HEPES buffer containing 40-60 mM NaCl, 7-12% sucrose, pH 7.3 containing glycerol. The final formulation was then filtered through a 0.2 μm filter and filled into glass vials, stoppered, capped and placed at −70±5° C. The frozen formulations were characterized for their mRNA content by HPLC or Ribogreen assay and percent encapsulation by Ribogreen assay, mRNA integrity by fragment analyzer, lipid content by high performance liquid chromatography (HPLC), particle size by dynamic light scattering on a Malvern Zetasizer Nano ZS, pH and osmolality.

### In-Cell Western (ICW)

96-well collagen plates were used to seed the cells at the appropriate density in Dulbecco's Modified Eagle Media (DMEM)/Fetal Bovine Serum (FBS) culture media. At the optimal confluence, cells were transfected with the targeted mRNAs diluted in the transfection reagent mix (MessengerMax and Opti-MEM). Cells were placed in a CO₂ incubator and allowed to grow. At the desired timepoint, media was removed, and cells were fixed in 4% fresh paraformaldehyde (PFA) for 20 min. After that, fixative was removed, and cells were permeabilized several times in Tris buffered saline with TWEEN (TBST) for 5 minutes each time. When permeabilization washes were complete, cells were incubated with a blocking buffer (ODYSSEY® Blocking Buffer (PBS) (Li-Cor, Lincoln, NE)) for 45 min. Primary antibody was then added and incubated for 1 hour at room temperature. Cells were then washed several times in TBST and incubated for 1 hour with a secondary antibody diluted in blocking buffer and containing a CellTag 700 stain. Cells were washed several times in TBST followed by a last wash in Tris-buffered saline (TBS). The plate was imaged using the Licor detection system, and data was normalized to the total number of cells labeled by the CellTag 700.

### Example 2: UTRs Screening in Hepa1,6 and Hep3B-Correlation at 24 h and 48 h

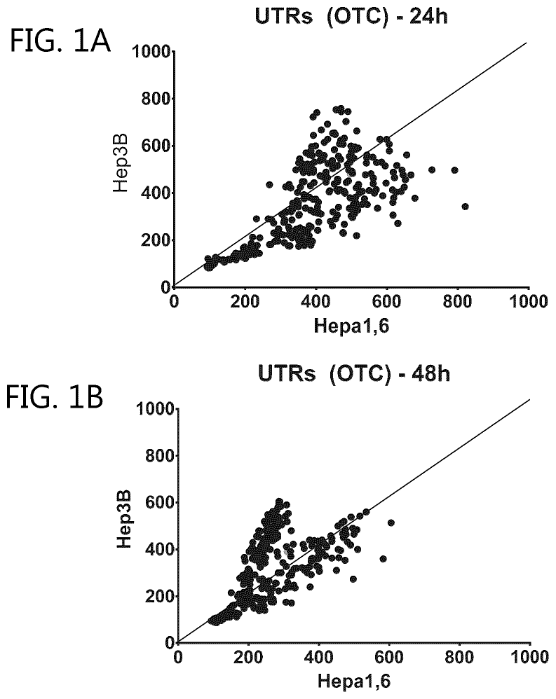

A UTR library was screened in vitro using mRNA construct #571 comprising the sequence of SEQ ID NO: 34 as the CDS (coding sequence). Hepa1,6 and Hep3B cells were transfected with the different mRNAs using commercially available transfection reagents and protein expression was measured, as described above (In-Cell Western assays, Example 1). OTC protein expression levels were measured by near-infrared fluorescent imaging systems. Commercially available antibodies for OTC were used for detection. Untransfected cells and reference sequences were used as internal controls. FIG. 1A is a scatter plot of OTC protein expression levels found in Hepa1,6 and Hep3B cells at 24 hours. FIG. 1B is a scatter plot of OTC protein expression levels found in Hepa1,6 and Hep3B cells at 48 hours. The aim of the screen was to determine a UTR-specific impact on OTC expression levels in a human (Hep3B) and a mouse (Hepa1,6) liver cell line to determine which UTRs would work best in both models and, in particular, to assess translatability from mouse-to-human. Top expressing UTRs were used in further profiling studies.

### Example 3: Round 1 of Protein Stability mRNA Compound Screening in Hepa1,6 and Hep3B at 24 h-Correlation

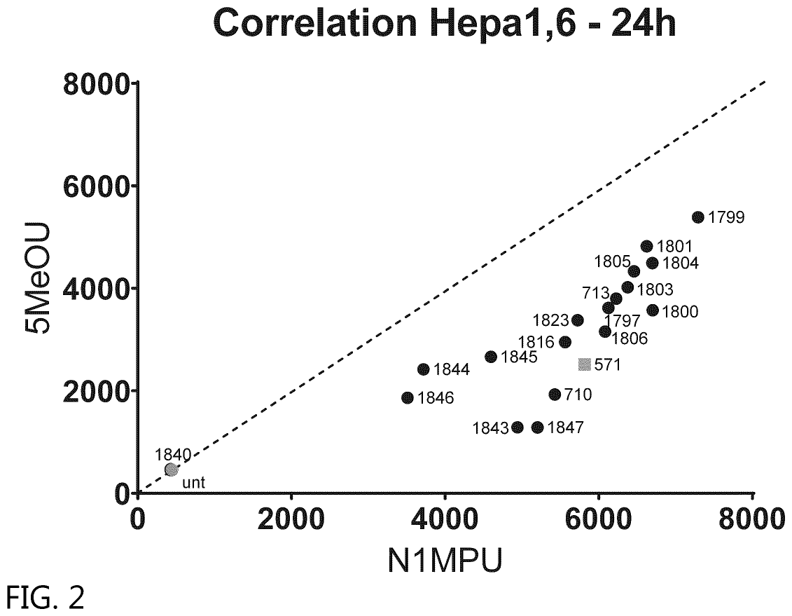

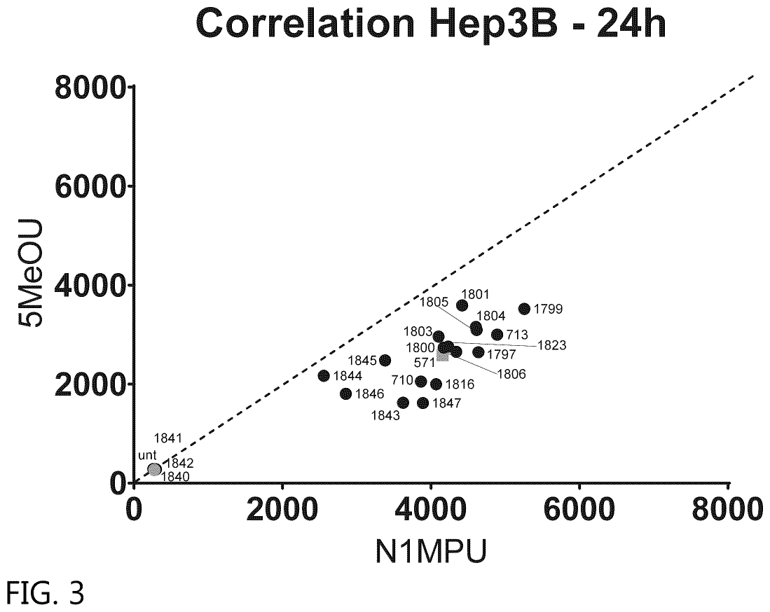

In vitro screening of certain mRNA constructs of Table 5 that were designed based on a protein-stability approach was performed. mRNA constructs with two different types of chemistries for the uridine residues were tested: N¹-methyl pseudouridine (N1MPU) and 5-methoxyuridine (5MeOU). In these experiments, 100% of the uridines in each mRNA were either N1MPU only or 5MeOU only (not a combination of 5MeOU or N1MPU). Hepa1,6 and Hep3B cells were transfected with the different mRNAs using commercially available transfection reagents and protein expression was measured, as described above (In-Cell Western (ICW) assays, Example 1). OTC protein expression levels were measured by near-infrared fluorescent imaging systems. Commercially available antibodies for OTC were used for detection. Untransfected cells and reference sequences were used as internal controls. FIG. 2 is a scatter plot showing the correlation of OTC protein expression levels in Hepa1,6 cells at 24 hours as a function of mRNAs tested and including N1MPU and 5MeOU chemistries. FIG. 3 is a scatter plot showing the correlation of OTC protein expression levels in Hep3B cells at 24 hours as a function of mRNAs tested and including N1MPU and 5MeOU chemistries. Shown in the figures is the degree of variability in expression levels for mRNAs with two different chemistries tested in a mouse and a human liver cell line. It can be seen that, in this experiment, most of the mRNA compounds express better when an N1MPU chemistry is used.

### Example 4: Round 2 of Protein Stability mRNA Compound Screening in Human Primary Hepatocytes at 24 h and 48 h-Correlation

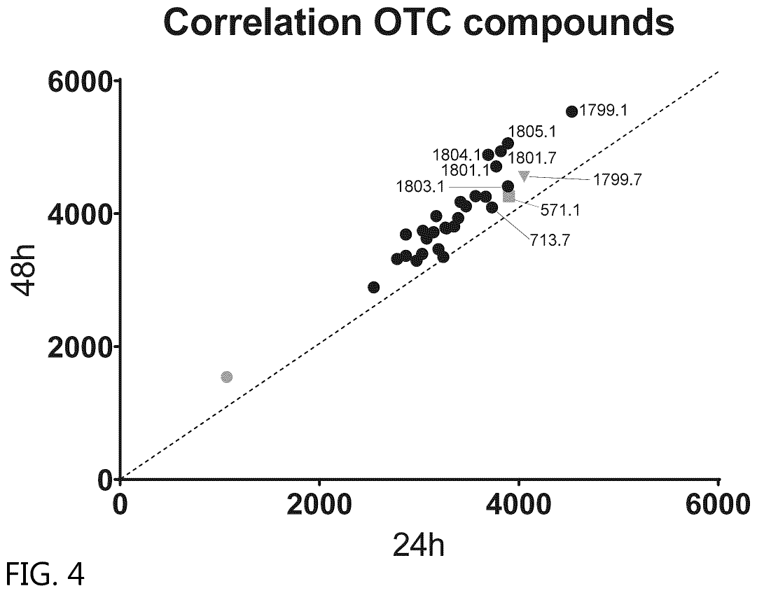

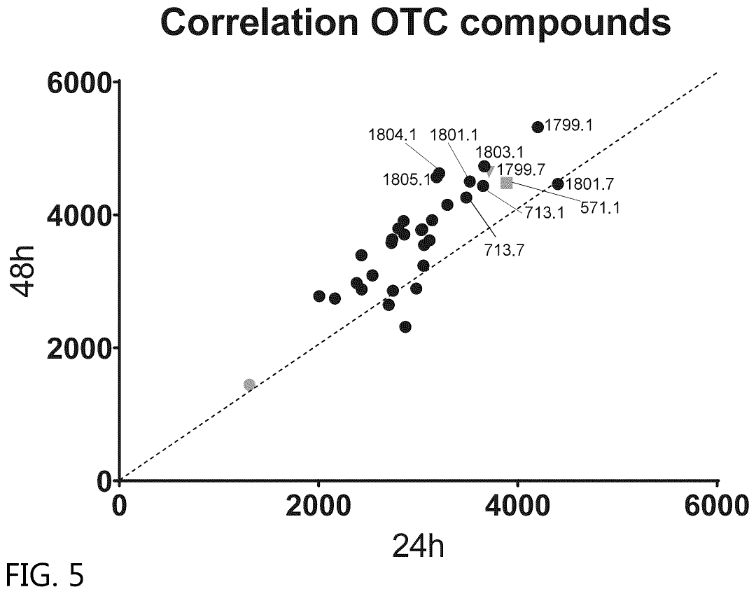

In vitro screening of certain mRNA constructs of Table 5 that were designed based on a protein stability approach was performed. mRNAs with two different chemistries were tested: 100% of the uridines being N1MPU, which constructs are indicated by the designation of the mRNA construct followed by “0.1” and 100% of the uridines being 5MeOU, which constructs are indicated by the designation of the mRNA construct followed by “0.7”. Human primary hepatocytes were transfected with the different mRNAs using commercially available transfection reagents and protein expression was measured, as described above (In-Cell Western (ICW), Example 1). OTC protein expression levels were measured by near-infrared fluorescent imaging systems. Commercially available antibodies for OTC were used for detection. Untransfected cells and reference sequences were used as internal controls (FIG. 4 and FIG. 5). The results indicate that, in contrast to the experiments conducted in cancer cell lines (Hepa1,6; Hep3B; Example 3), mRNAs with both chemistries expressed similarly in human primary hepatocytes.

### Example 5: Round 3 of Protein Stability Compound Screening in Human Primary Hepatocytes at 24 h and 48 h-Correlation

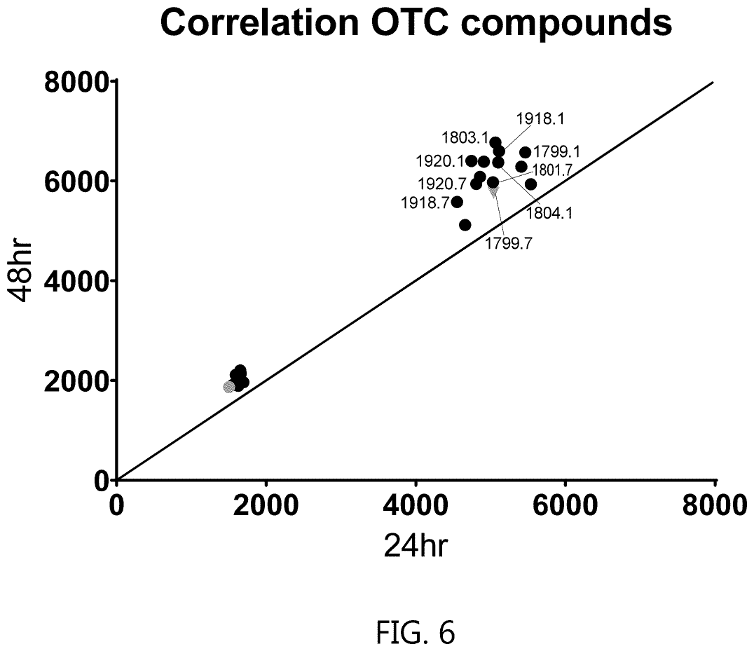

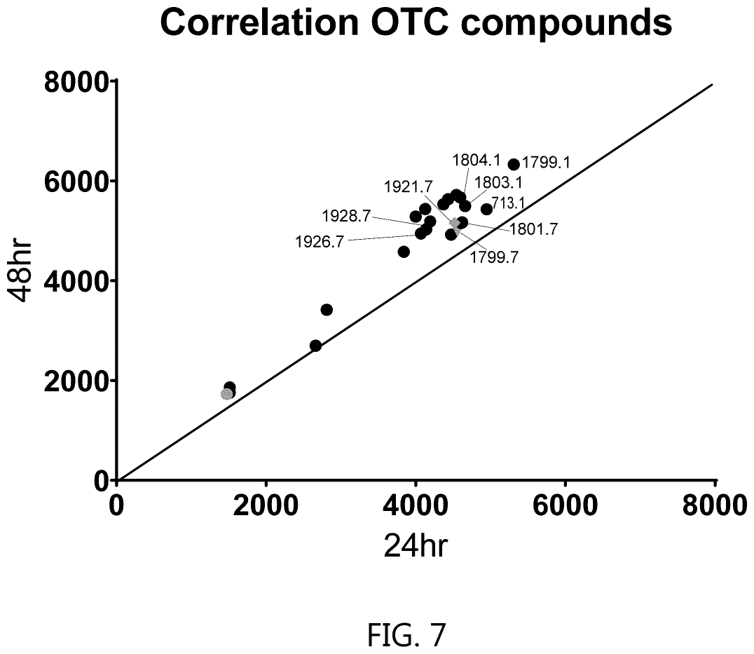

An in vitro screen of novel compounds designed based on a protein stability approach was performed. mRNAs with two different chemistries were tested, with N1MPU indicated by the name of mRNA constructs followed by “0.1” and 5MeOU indicated by the name of mRNA constructs followed by “0.7”. Human primary hepatocytes were transfected with the different mRNAs using commercially available transfection reagents and protein expression was measured, as described above (In-Cell Western (ICW) assays, Example 1). OTC protein expression levels were measured by near-infrared fluorescent imaging systems. Commercially available antibodies for OTC protein were used for detection. Untransfected cells and reference sequences were used as internal controls (FIG. 6 and FIG. 7). The results indicate that, in contrast to the experiments conducted in cancer cell lines (Hepa1,6; Hep3B; Example 3), mRNAs with both chemistries expressed similarly in human primary hepatocytes.

### Example 6: OTC Protein-Expression Levels in Human Primary Cells Transfected with OTC mRNA Constructs 1799.7 (5MeOU Chemistry) Encoding the OTC Protein of SEQ ID NO: 3 and 1921.7 (5MeOU Chemistry) Encoding the Modified OTC Protein of SEQ ID NO: 4

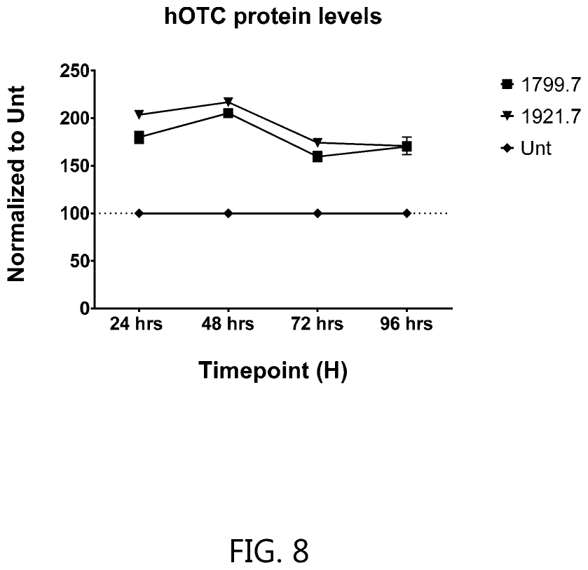

Human primary hepatocytes were transfected with the different mRNAs using commercially available transfection reagents and protein expression was measured, as described above (In-Cell Western (ICW) assays, Example 1). OTC protein expression levels were measured by near-infrared fluorescent imaging systems during a time course study of up to 96 hours. Commercially available OTC antibodies were used for detection. Untransfected cells were used as internal control. The resultant plot shows OTC protein levels normalized to untransfected controls (FIG. 8). The purpose of this study was to evaluate the half-life of the unmodified versus the modified protein sequence (encoded by constructs 1799.7 and 1921.7, respectively) under in vitro conditions in transfected human primary hepatocytes. The results indicate that 1921.7 demonstrated more stable expression than 1799.7.

### Example 7: OTC Expression Levels Measured by Multiple Reaction Monitoring (MRM) Mass Spectrometry in Spf/Ash Mice Dosed at 10 mg/kg

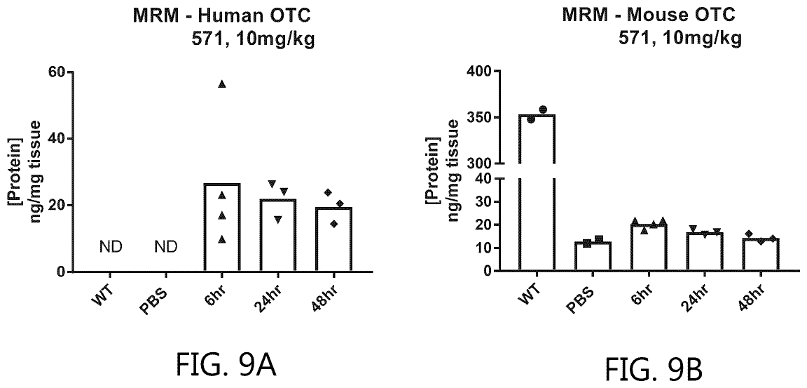

Spf/ash mice received an IV injection with either PBS or lipid-formulated (as described in Example 1) hOTC-mRNA at a 10 mg/kg dose level. WT mice were used as internal controls to determine endogenous levels. A time course (6 hours, 24 hours and 48 hours) was performed, and expression levels were measured by MRM using human- and mouse-specific epitopes for OTC. Human- and mouse-specific heavy peptides were designed to measure total levels of OTC from both species. Graphs represent the amount of protein (ng/mg tissue) detected by MRM specific for human OTC (FIG. 9A) or mouse OTC (FIG. 9B). This data set shows that quantitative levels of human OTC (hOTC) were observed in treated mice that derived from translation of the delivered mRNAs. A high level of quantifiable hOTC protein was seen up to 48 hours.

### Example 8: OTC Expression Levels Measured by Western Blot in Mice Dosed at 3 mg/kg

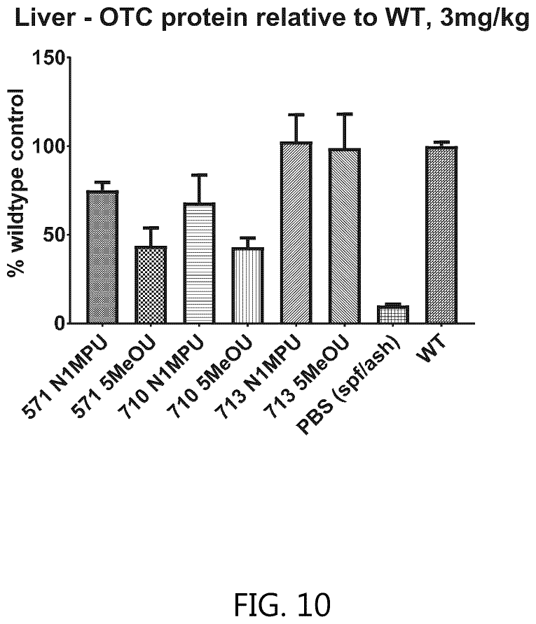

Spf/ash mice received an IV injection with either phosphate buffered saline (PBS) or lipid-formulated OTC-mRNAs at a 3 mg/kg dose using two different uridine nucleotide chemistries (N1MPU and 5MeOU). WT mice were used as internal controls to determine endogenous levels. Animals were sacrificed 24 hours post-dose. OTC expression levels were measured by Western Blot (WB) using an OTC specific antibody. In the results provided in FIG. 10, the bars represent the percentage of expression relative to WT levels (100%). The data shows that WT levels of total OTC were achieved for several codon-optimized sequences in a mouse background.

### Example 9: OTC Expression Levels Measured by MRM in a Dose Range-Finding Study

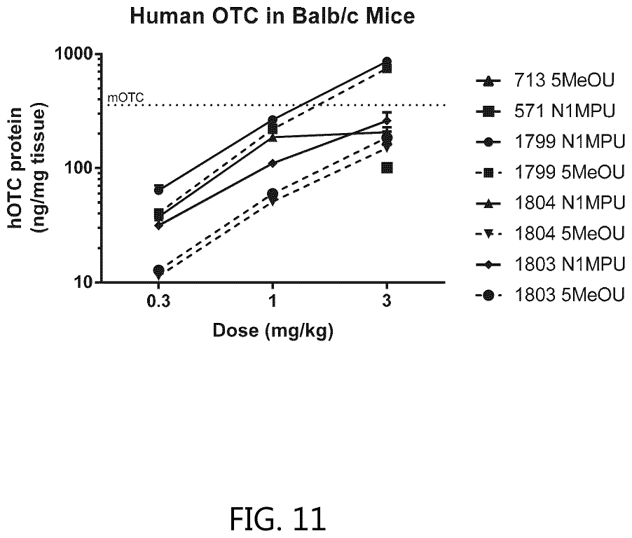

Balb/c mice received an IV injection with either PBS or lipid-formulated OTC-mRNAs at three different doses (0.3 mg/kg, 1 mg/kg and 3 mg/kg) and using two different uridine chemistries (N1MPU and 5MeOU). Animals were sacrificed 24 hours post-dose and expression levels were measured by MRM using human and mouse specific epitopes for OTC. MRM was used to quantitatively determine human-specific and mouse-specific OTC protein levels (FIG. 11). The graph in FIG. 11 shows expression of human OTC in Balb/c mice (ng per mg of liver tissue). The horizontal dotted line represents relative mouse OTC levels in Balb/c mice (FIG. 11). Expression levels of human OTC (hOTC) protein for mRNA construct 713 using 5MeOU and mRNA construct 571 using N1MPU are shown in FIG. 11. The data generated in this figure shows that WT levels of human OTC were achieved with the codon-optimized sequences disclosed herein in a dose-dependent manner in a mouse background.

### Example 10: Urinary Orotate Levels Measured in PBS—and mRNA-Treated Spf/Ash Mice

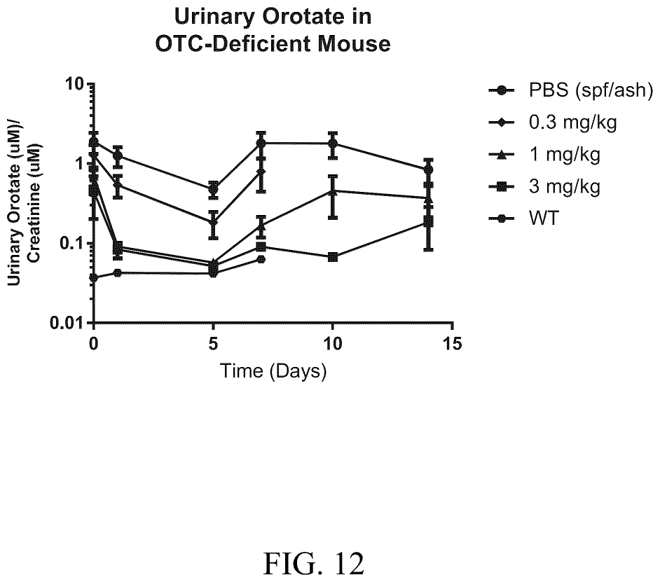

Spf/ash mice received an IV injection with either PBS or lipid-formulated OTC-mRNA construct 1799.7 (5MeOU chemistry) at three different doses: 0.3 mg/kg, 1 mg/kg and 3 mg/kg. Urinary orotate levels were determined in untreated wild-type (WT) and both untreated and treated Spf/ash mice. A urinary orotate time course was performed in Spf/ash and WT mice, and urinary orotate levels were measured at each timepoint. The results can be seen in FIG. 12. Urinary orotate was normalized to creatinine, which is represented in the graph on the y-axis throughout the time course and served as a proof-of-concept of functional restoration of OTC activity post-injection. At 3 mg/kg, a sustainable reduction of urinary orotate levels was observed for up to 14 days, with reduced urinary orotate levels in treated Spf/ash mice comparable to urinary orotate levels in WT mice.

### Example 11: Pharmacokinetic/Pharmacodynamic (PK/PD) Analysis Comparing Human OTC Expression Levels and Urinary Orotate at 96 Hours Post Dose

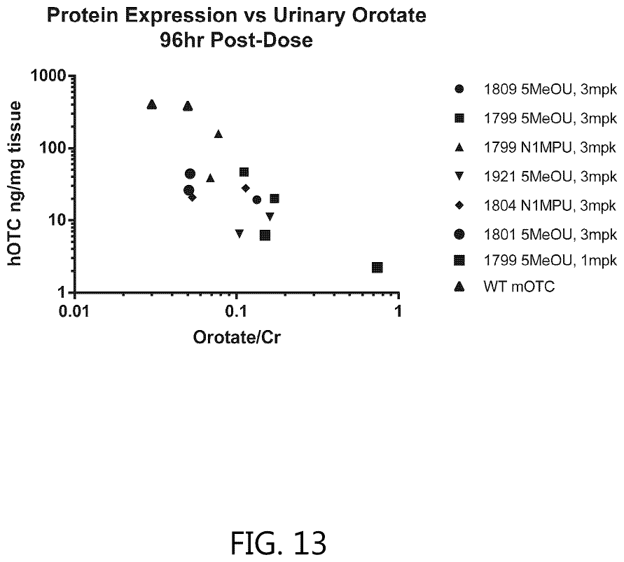

Spf/ash mice received an IV injection with either PBS or certain lipid-formulated OTC-mRNAs from Table 5 at 1 mg/kg and 3 mg/kg using two different uridine chemistries (N1MPU and 5MeOU). WT mice were used as internal controls. Human-specific OTC levels were measured by MRM, and urinary orotate was determined in each sample and normalized to creatinine. The resultant PK/PD profile is plotted in FIG. 13 and the PK/PD analysis shows the correlation between protein expression levels and reduction of urinary orotate in a compound-specific manner. Construct 1799.7 (5MeOU chemistry) showed a high PK/PD correlation.

### Example 12: Fractioning of Spf/Ash Mice In Vivo Samples Treated with Selected mRNAs

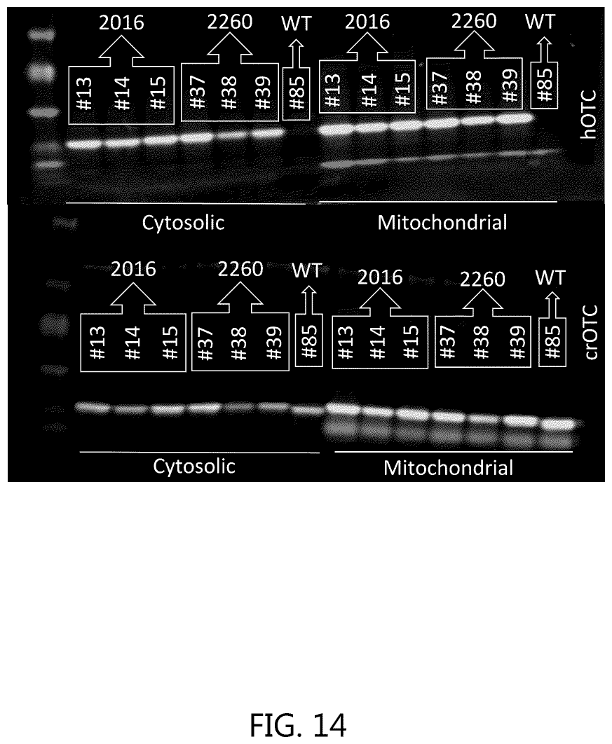

Spf/ash mice received an IV injection with either PBS or lipid-formulated (as described in Example 1) OTC-mRNAs at 1 mg/kg and 3 mg/kg. WT mice were used as internal controls. Livers were harvested from the mice and sample fractionation was performed on the liver samples, separating the cytosolic and mitochondrial fractions. OTC levels were measured by Western Blot (WB) using human specific (hOTC) and crossreactive (crOTC) antibodies (FIG. 14). Cyclooxygenase IV (CoxIV) was used as a mitochondrial control. OTC protein expression levels were measured by near-infrared fluorescent imaging systems and normalized to total protein. The WB results indicate differences in OTC expression levels within mitochondrial and cytosolic fractions when the 2016 and 2260 mRNA constructs were delivered in the Spf/ash mice. These results indicate that both compounds can efficiently target the mitochondria.

### Example 13: Plot of the mitochondrial vs cytosolic fractions of Spf/ash mouse samples treated with mRNA constructs 2016 and 2260

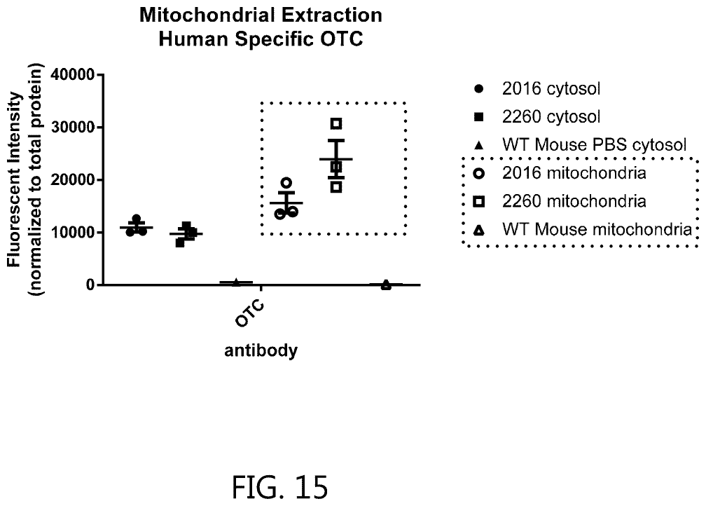

Spf/ash mice received an IV injection with either PBS or lipid-formulated OTC-mRNAs at 3 mg/kg. WT mice were used as internal controls. Sample fractionation was performed on liver samples harvested from the mice, separating the cytosolic and mitochondrial fractions. OTC levels were measured by Western Blot using a human specific antibody. OTC protein expression levels were measured by near-infrared fluorescent imaging systems and both fractions, normalized to total protein, were plotted (FIG. 15). The plot of protein expression levels shown in FIG. 14 (Example 12) indicates that even though both compounds, 2016 and 2260, deliver similar protein levels in the cytosol, 2260 delivers more human OTC than 2016 in the mitochondria. The 2260 compound includes a modified mitochondrial signaling peptide sequence provided herein.

### Example 14: Urinary Orotate Levels in Spf/Ash Mice Treated with mRNA Construct 2260

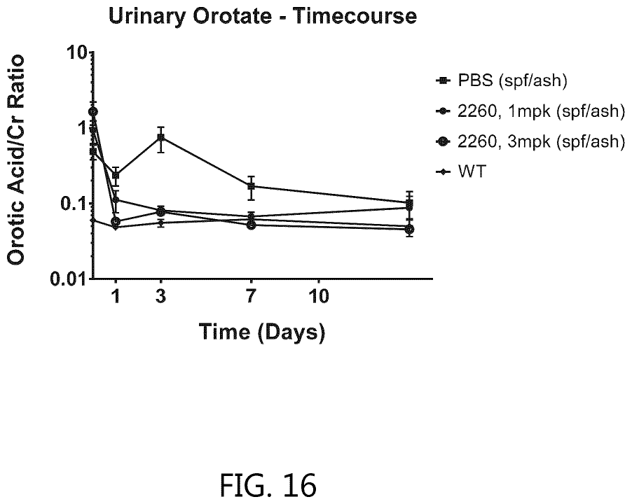

Spf/ash mice received an IV injection with either PBS or lipid-formulated (as described in Example 1) OTC-mRNA at 1 mg/kg and 3 mg/kg. WT mice were included as an internal control. Urinary orotate levels were measured at 0, 1, 3, 7 and 14 days, and levels were normalized to creatinine (FIG. 16). The functional read-out of this assay shows that urinary orotate levels were reduced for up to 14 days with compound 2260 in a dose-dependent manner.

### Example 15: Survival of Spf/Ash Mice on a High Protein Diet During Treatment with OTC-mRNA Construct 1799.7 (5MeOU Chemistry)

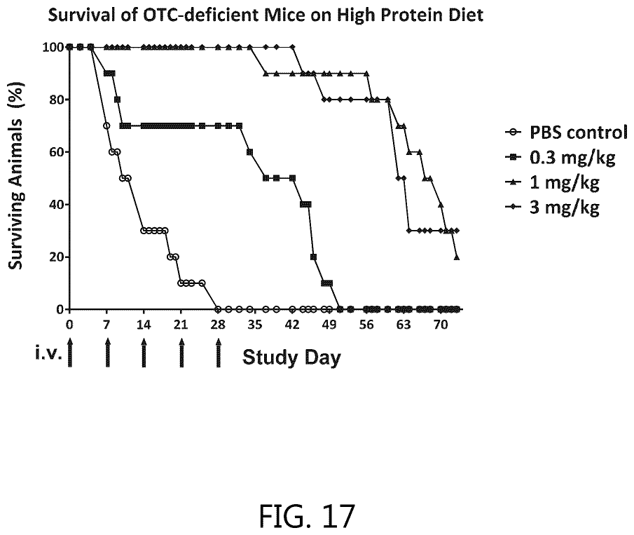

Spf/ash mice received an IV injection with either PBS or lipid-formulated OTC-mRNA (1799.7) at three doses: 0.3 mg/kg, 1 mg/kg and 3 mg/kg. Mice were fed a high protein diet from day 0 to the end of the study. Treated animals were injected intravenously on days 0, 7, 14, 21 and 28 (indicated by the arrows in the chart in FIG. 17). Survival rates were determined every week. The plot in FIG. 17 summarizes the entire study timeline and the survival rates observed for the different groups. The results show that animals treated with human OTC mRNAs described herein displayed greater survival during a hyperammonemic crisis, suggesting a protective role of OTC mRNAs described herein in detoxifying the animals from toxic ammonia. The survival rate was dose-dependent, and animals treated with a 3 mg/kg dose had a higher survival rate than animals treated at 1 mg/kg or 0.3 mg/kg.

### Example 16: Comparison of hOTC Expression Levels from mRNAs with Different Modifications

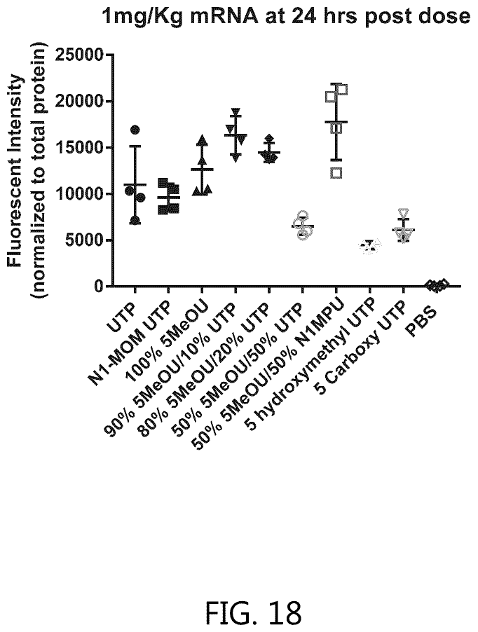

Doses of lipid-formulated OTC-mRNA construct 2262 were injected intravenously into 8 to 10 week-old female Balb/c mice at a dose of 1 mg/kg. Different chemistries were used in this study as indicated in the bottom (x) axis of the chart provided in FIG. 18. Mouse livers were harvested at 24 hours post IV-administration, and western blotting was performed using a hOTC specific antibody. Levels were normalized to total protein (FIG. 18). Each construct was formulated as a lipid nanoparticle comprising an ionizable cationic lipid as described in Example 1. This dataset shows the impact of different uridine chemistries on the expression levels of the codon-optimized mRNAs described herein.

### Example 17: Lipid Formulation Development Studies

Lipid formulations were prepared as in Example 1 to evaluate ionizable cationic lipids in the lipid formulations for their physicochemical properties: particle size (desired to be 55 to 85 nm), polydispersity index (should not be more than 0.2) and percent encapsulated mRNA (not less than 85%). The physicochemical properties of the formulations are summarized in Table 7 below:

TABLE 7
Analytical Data Summary of Lipid Formulations

| | | | Poly- | |
|---|---|---|---|---|
| | | Particle | dispersity | % Encapsulated |
| Lipid | Batch ID | Size (nm) | Index | mRNA |
| Lipid # 1 | YB17_00506 | 80.5 | 0.06 | 95.2 |
| Lipid # 2 | YB17_00507 | 67.8 | 0.09 | 95.5 |
| Lipid # 3 | YB17_00508 | 72.7 | 0.08 | 95.5 |
| Lipid # 7 | YB17_00510 | 76.6 | 0.09 | 95.4 |

Assessment of formulation physicochemical properties (particle size, polydispersity index and percent encapsulated mRNA) in combination with in vivo potency (as measured by protein expression) led to the selection of three lipids (Lipid #2, Lipid #3, and Lipid #7) for further evaluation with OTC mRNA.

Parallel formulation development studies were performed that focused on determining optimal molar percentages of the ionizable cationic lipid, DSPC, Cholesterol and PEG2000-DMG in the formulation, utilizing Lipid #7 as the tool lipid, based on a Design of Experiments (DOE) approach. The study evaluated Lipid #7 and DSPC ratios with a fixed PEG2000-DMG ratio at 1.5%. The PEG2000-DMG ratio was fixed to 1.5% to maximize mRNA encapsulation and in vivo potency. The DOE study focused on the assessment of formulation physicochemical properties: particle size (55 to 85 nm), polydispersity index (not more than 0.2) and percent encapsulated mRNA (not less than 85%).

The measured physicochemical properties of the lipid formulations from the DOE study are outlined in Table 8 below:

TABLE 8
Analytical Data Summary of Lipid Formulations for DOE Study

| | | Lipid Molar Ratios (%) | | | |
|---|---|---|---|---|---|
| | | [Lipid # | | | |
| | | 7:DSPC:CHOL:PEG2000- | 1F/T Data at 0.06 mg/mL | | |
| DOE | | DMG], | Particle | | % encapsulated |
| Run # | Batch ID | CHOL level | Size (nm) | PDI | mRNA |
| 1 | YB18_00783 | [42:13:43.5:1.5], high | 66.9 | 0.19 | 96.5 |
| 2 | YB18_00784 | [50:10:38.5:1.5], medium | 68.8 | 0.18 | 95.0 |
| 3 | YB18_00786 | [42:7:49.5:1.5], high | 67.1 | 0.24 | 93.5 |
| 4 | YB18_00787 | [50:13:35.5:1.5], medium | 70.0 | 0.18 | 95.3 |
| 5 | YB18_00788 | [58:13:27.5:1.5], low | 74.9 | 0.13 | 93.2 |
| 6 | YB18_00789 | [42:10:46.5:1.5], high | 71.9 | 0.21 | 95.8 |
| 7 | YB18_00790 | [50:7:41.5:1.5], medium | 72.2 | 0.16 | 95.4 |
| 8 | YB18_00791 | [58:10:30.5:1.5], low | 83.7 | 0.14 | 92.2 |

Based on the data above, the molar percentage of Lipid #7 played an important role in the physicochemical properties of the formulation. Most notably, formulations containing 42% of Lipid #7 had the highest PDI (with two out of three formulations outside of the acceptable range), while formulations containing 58% of Lipid #7 had the lowest PDI. Conversely, formulations containing 58% of Lipid #7 had the greatest particle size, but were still within the acceptable range. The percent encapsulated mRNA for all formulations were within the acceptable range, with formulations containing 42% or 50% of Lipid #7 having slightly higher % encapsulated mRNA.

### Example 18: Comparison of the Effect of Ionizable Cationic Lipids on OTC Expression in Balb/c Mice

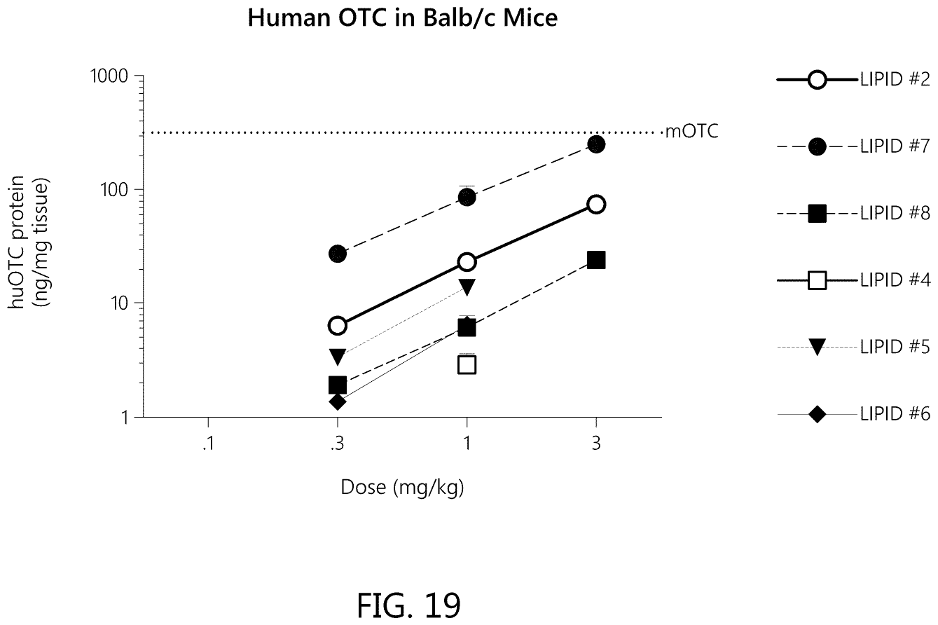

Lipid formulations were prepared as in Example 1, using the 571 mRNA construct for each of Lipid #2, Lipid #7, Lipid #8, Lipid #4, Lipid #5, and Lipid #6 to test the dose-dependent effect of these lipids on the expression of hOTC in Balb/c mice. Formulations of each of these lipids were administered intravenously to the mice at doses of 0.3 mg/kg, 1.0 mg/kg, and 3.0 mg/kg. The expression levels are shown relative to wild-type mouse OTC (mOTC) expression levels in FIG. 19. As can be seen in this figure, each of the formulations showed dose-dependent expression, with increasing concentrations of hOTC seen as doses increased. The formulations comprising Lipid #2 and Lipid #7 showed the greatest levels of hOTC expression, with Lipid #7 showing about 3 to 4-fold greater expression than Lipid #2.

### Example 19: Further Comparisons of the Effect of Ionizable Cationic Lipids on OTC Expression

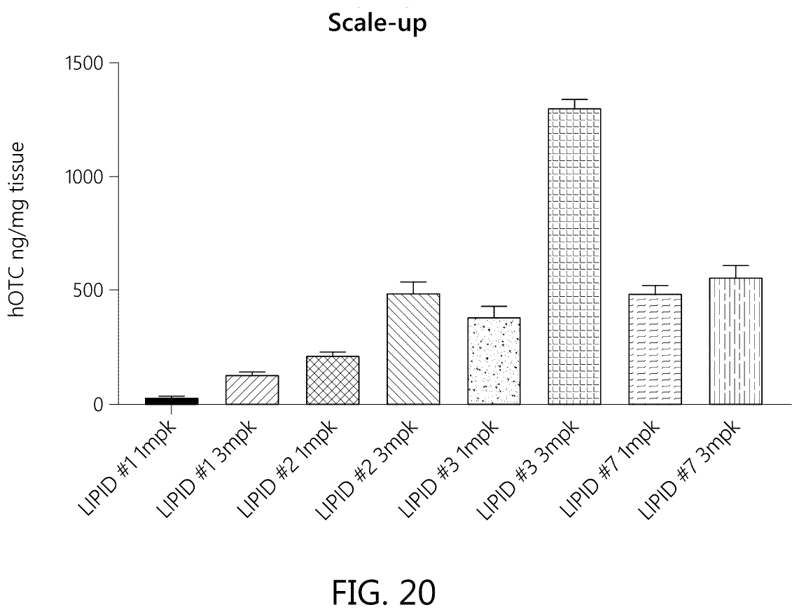

Further lipid formulations were prepared as in Example 1, using the 571 mRNA construct for each of Lipid #1, Lipid #2, Lipid #3, and Lipid #7 to test the dose-dependent effect of these lipids on the expression of hOTC in Balb/c mice. Formulations of each of these lipids were administered intravenously to the mice at doses of 1.0 mg/kg and 3.0 mg/kg. The expressions levels are shown in FIG. 20. As can be seen, there was a dose-related increase in hOTC expression. Lipid #3 showed an exceptional dose-response as the dose increased, while Lipid #7 appeared to have only a small dose response.

### Example 20: Survival Rate of Spf/Ash Mice for Lipid Formulations Using Different Ionizable Cationic Lipids

The pharmacology of the OTC mRNA lipid formulations was investigated in studies that elucidated their efficacy and potential mechanism of action in a series of studies in the hemizygous male Spf/ash mouse model. These mice possess a genetic mutation on the X-chromosome (hypomorphic model) that causes abnormal splicing of the endogenous OTC mRNA, resulting in less than 10% of residual OTC enzymatic activity in the liver. The hypomorphic Spf/ash mouse shares similar biochemical features with OTCD patients, including elevated concentrations of urinary orotic acid (orotate), blood glutamine, and decreased concentrations of blood citrulline and arginine. The hemizygous male Spf/ash mice used in the pharmacology studies were derived by crossing B⁶EiC3Sn a/A-Otcspfash/J Het mice x B⁶EiC3SnF1/J mice as homozygous mice are not viable. The heterozygous male B⁶EiC3SnF1/J mice were used as wildtype (WT) controls.

Spf/ash mice do not typically display severe hyperammonemia and reduced ureagenesis. Acute disruption of their urea acid cycle was induced by a bolus administration of ammonium chloride (NH₄Cl), resulting in hyperammonemia and reduced ureagenesis. In addition, their urea cycle was also stressed by maintaining these mice on a high protein diet (HPD).

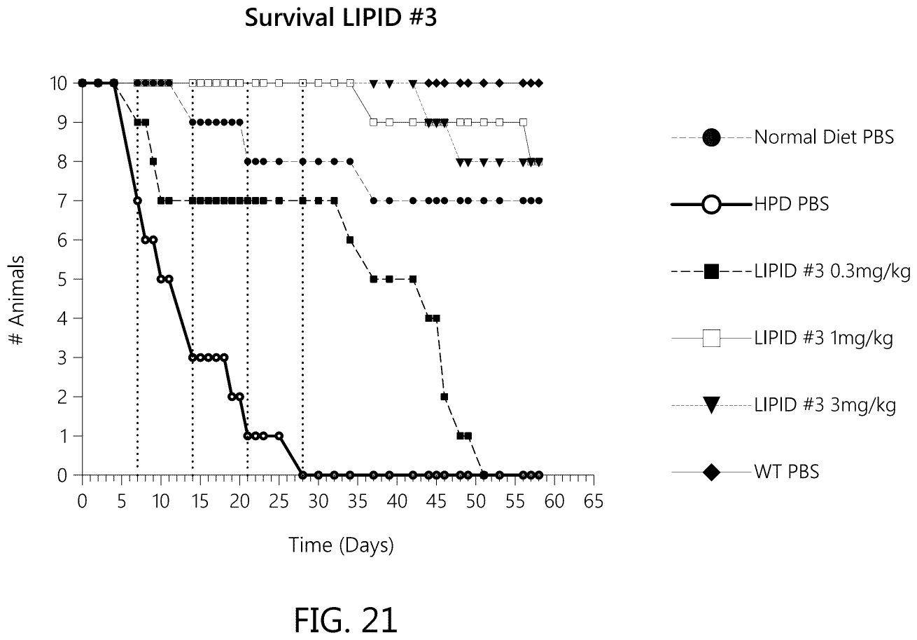

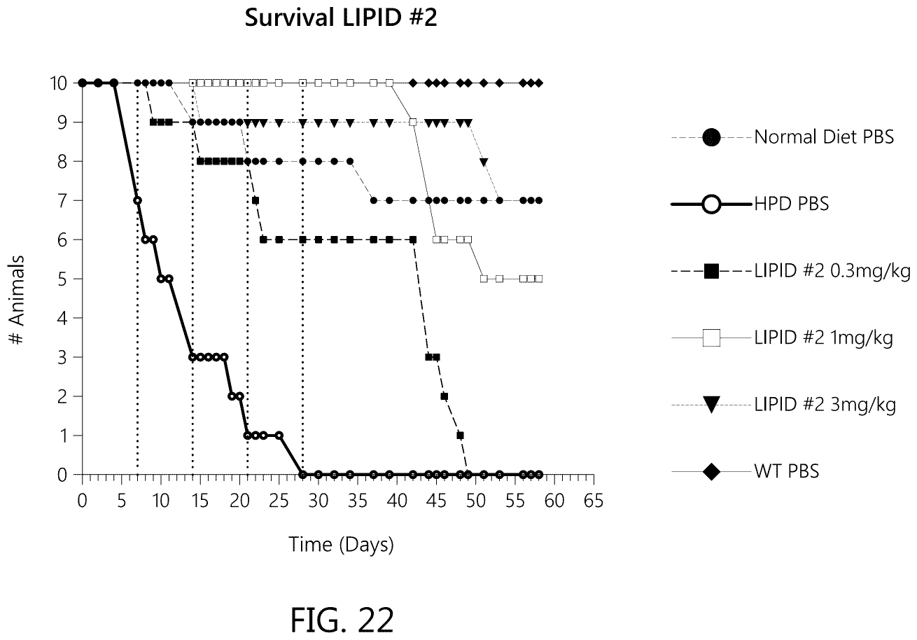

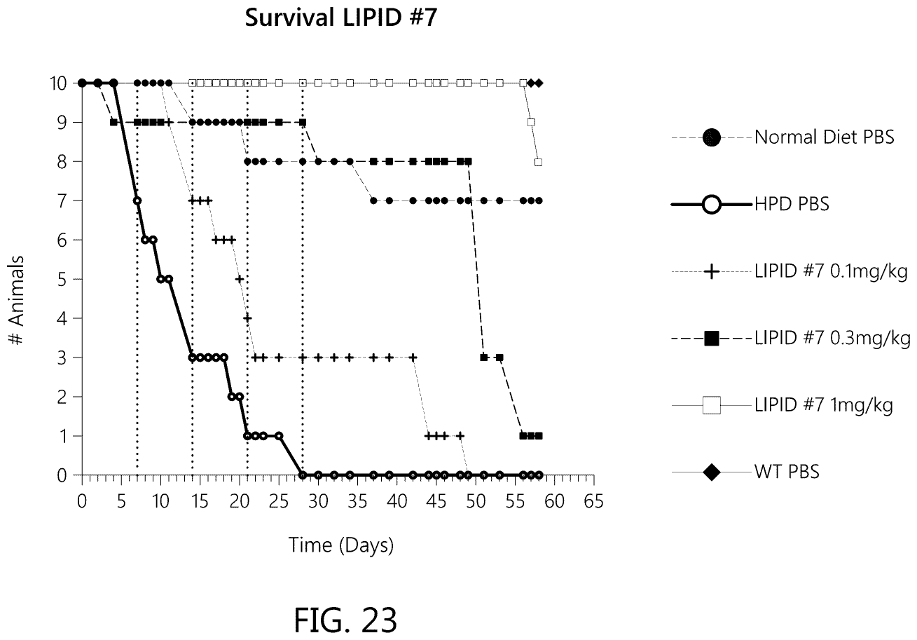

To study the effect of different ionizable cationic lipids on survival rate, Spf/ash mice received an IV injection with either PBS or one of three different lipid-formulated OTC-mRNA (1799.7) compositions. The three different compositions were prepared as in Example 1 above, and had either Lipid #3, Lipid #2, or Lipid #7. The formulations were administered at three doses: 0.3 mg/kg, 1 mg/kg and 3 mg/kg for the Lipid #2 and Lipid #3 formulations and 0.1 mg/kg, 0.3 mg/kg, and 1.0 mg/kg for the Lipid #7 formulation. Mice were on a high protein diet from day 0 to the end of the study. Treated animals were injected by intravenously on days 0, 7, 14, 21 and 28 (indicated by the dashed lines in the charts in FIGS. 21-23). Survival rates were determined daily. The plots in FIGS. 21-23 summarize the entire study timeline and the survival rates observed for the different groups. The results show that animals treated with human OTC mRNAs described herein displayed greater survival during a hyperammonemic crisis, suggesting a protective role of OTC mRNAs described herein in detoxifying the animals from toxic ammonia. The survival rate was dose-dependent.

Upon exposure to a high protein diet, mortality was observed as early as Day 4 (Lipid #7, 0.3 mg/kg). A high protein diet and treatment with PBS resulted in a steep mortality rate of Spf/ash mice beginning on Day 7, with 100% mortality by Day 28. Low dose treatments significantly extended the survival rate by at least two weeks, with 100% mortality observed on Day 49 for Lipid #2, Day 51 for Lipid #3, and Day 49 for Lipid #7, respectively. Mid and high dose treatments resulted in 90-100% survival rates throughout the dose administration period, with Lipid #7 at 1 mg/kg achieving a 100% survival rate. Dose-related improvement of survival rates was also seen from Day 28 through Day 70, the “washout period.” At the high dose levels, Spf/ash mice were protected from hyperammonemia-induced death for at least two weeks after the last dose. At the mid and high dose levels, 100% mortality was not seen for a monitoring period out to six weeks after the last dose. At both the 0.3 mg/kg and 1.0 mg/kg levels, Lipid #7 was the most efficacious in prolonging survival, followed by Lipid #3, then Lipid #2. For the 3.0 mg/kg level, Lipid #3 was more efficacious than Lipid #2.

### Example 21: Lipid Formulations Tolerability Study

To test the tolerability of OTC mRNA lipid formulations, lipid formulations as prepared in Example 1 using mRNA construct 1799.7 and either Lipid #2, Lipid #3, or Lipid #7 were tested in CD-1 mice at high dosages. All animals received an intravenous bolus treatment at a dosing volume of 10 mL/kg, once a week on Day 0, Day 7, Day 14, Day 21, and Day 28 of the study. Animals were observed daily for morbidity and mortality and body weights were determined twice weekly. Blood was collected from all animals for the measurement of ALT, AST and serum cytokines at 6 hours after the first and last dose. Animals were terminated 72 hours after the final dose (Day 31) for gross observation, measurement of whole body and organ weights (liver and spleen), tissue collection for histopathology (liver, kidney, spleen, heart and lung) and blood collection for analysis of cytokines, hematology and clinical chemistry.

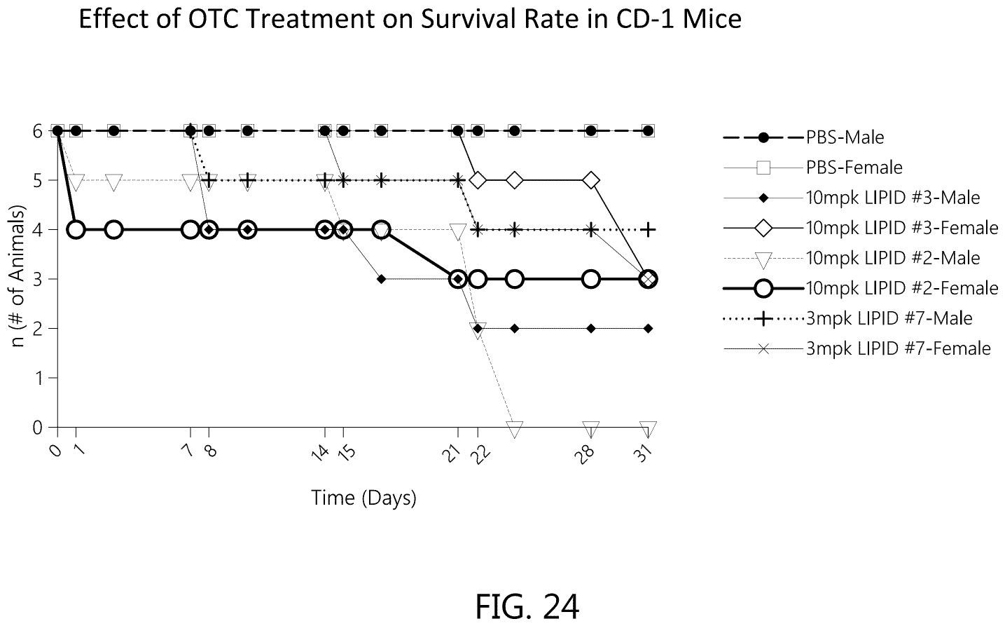

The survival rate results from this study are depicted in FIG. 24, which shows the dose-dependent effect of OTC mRNA lipid formulation treatment on survival rate after multiple dose administrations. A 100% survival rate was observed at the 3 mg/kg dose level for both Lipid #3 and Lipid #2 and at 1 mg/kg for Lipid #7 (in both genders). Males appeared to be more sensitive than females for Lipid #3 and Lipid #2, with 100% mortality observed in the Lipid #2 10 mg/kg male group. Furthermore, mortality was observed at the 10 mg/kg dose level for Lipid #3 and Lipid #2, and at the 3 mg/kg dose level for Lipid #7. At 10 mg/kg, Lipid #3 was better tolerated than Lipid #2. Data resulting from this study suggested a ranking of Lipid #3>Lipid #2>Lipid #7 for tolerability.

A full assessment of the in vivo tolerability study (including body weight, ALT/AST, and cytokine analysis) was also performed. A dose-dependent trend in body weight effects was observed that corresponded to survival. However, there did not appear to be a biologically meaningful effect on overall body weight gains throughout the study. Hematology parameters were evaluated 72 hours after the final dose. No meaningful effects were observed for RBC, Hg, HCT, MCV, MCH, lymphocytes, monocytes, white blood cells. While minor changes were observed, due to variability among animals and lack of dose dependency, they could not be attributed to the administration of the OTC mRNA lipid formulations. A trend for increases in eosinophils and basophils and an increase in basophils for Lipid #3 at 10 mg/kg was observed.

Serum cytokines were evaluated both at 6 hours after the first and final dose and 72 hours after the final dose. Significant dose-dependent increases (for some cytokines >100-fold) were observed 6 hours after the first and final dose for TNF-α, IL1-β, IFNγ, MCP-1, IL-6, and IFNα for all lipid-formulated OTC mRNA-treated animals that returned to baseline 72 hours after the final dose, with the exception of TNF-α and IFNγ for Lipid #7 at 3 mg/kg. While there were other changes observed at the 72-hour post dose time point, due to variability within the dose group and a lack of dose dependency, they were not attributed to the administration of the OTC mRNA formulation.

### Example 22: In Vivo Tissue Lipid Clearance Studies

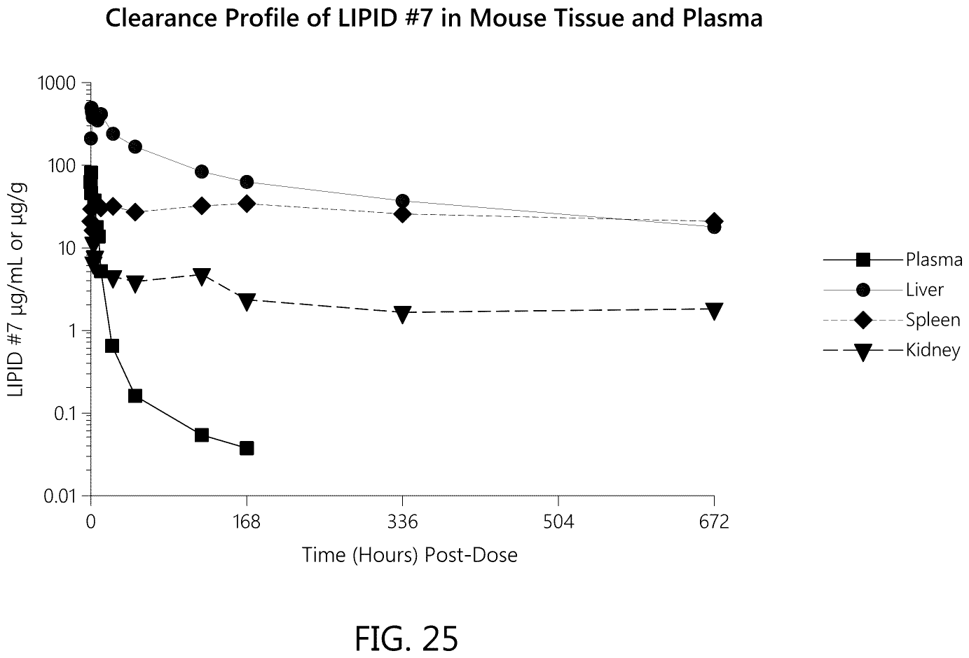

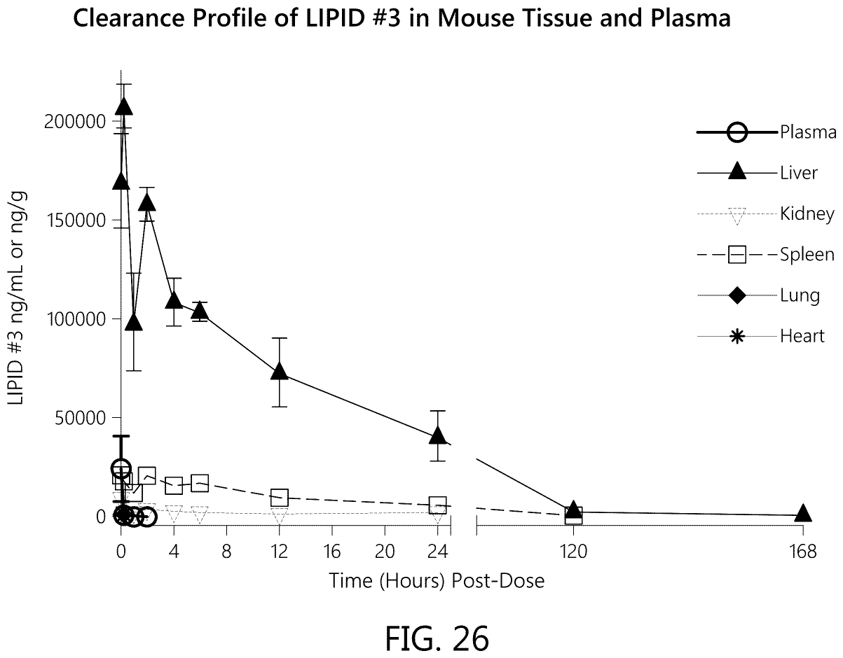

Additional studies were conducted to evaluate the in vivo tissue clearance profile of Lipid #7 and Lipid #3 in Balb/c mice. The clearance profiles in mouse tissue and plasma can be found in FIG. 25 (for Lipid #7) and FIG. 26 (for Lipid #3). All animals received a single intravenous bolus treatment of 1 mg/kg of the Lipid #7 formulation at a dosing volume of 10 mL/kg. Plasma, liver, kidney, and spleen were collected from mice (n=3 mice per timepoint) at 0, 2, 15, 30 minutes; 1, 2, 4, 6, 8,12, 24, 48 hours; and 5, 7, 21, 28 days post-dose. All samples were analyzed for Lipid #7 concentrations.

The highest level of Lipid #7 was observed in the liver, with a ranking of liver >spleen>plasma>kidney. In the plasma, Lipid #7 was no longer detected by 14 days post-dose. By 28 days post-dose (the last time point evaluated), Lipid #7 was detected at a concentration of 18,433 μg/mL in the spleen, 17,233 μg/mL in the liver and 2,340 pg/mL in the kidney. The half-life for Lipid #7 was determined to be 57 hours for plasma, 84 hours for liver, and 234 hours for kidney. Tissue half-life of Lipid #7 could not be determined for the spleen.

All animals received a single intravenous bolus treatment of 1 mg/kg of the Lipid #3 formulation at a dosing volume of 10 mL/kg. Plasma was collected from mice (n=3 per timepoint) at 0.25, 1, 2, 4, 6, 12, 24, 120, and 168 hours post-dose. Liver, kidney, and spleen were collected from mice (n=3 per timepoint) at 0.033, 0.25, 1, 2, 4, 6, 12, 24, 120, and 168 hours; 1, 2, 3, 4-weeks post-dose. Heart, brain and lungs were collected from mice (n=3 per timepoint) at 0.25 hours; 1, 2-weeks post-dose. Only samples from 0.25 hrs to 1-week post-dose were analyzed for Lipid #3 tissue concentrations.

The highest level of Lipid #3 was observed in the liver, with a ranking of liver>plasma>spleen>kidney>lung>heart>brain. In the plasma, Lipid #3 was no longer detected by 4 hours post-dose. Lipid #3 was not detected in brain tissue at either time point post-dose. Lipid #3 Tmax occurred at 0.033 to 1 hour post-dose in kidney, 0.033 to 1-hour post-dose in liver, and 1 to 2 hours post-dose in spleen. Because only two time points were assessed for heart and lung, the Tmax of Lipid #3 could not be determined. Lipid #3 concentrations persisted through 168 hours post-dose in liver and 120 hours post-dose in all other tissues, but were measured close to the lower limit of quantitation (LLOQ) of the assay. The half-life for Lipid #3 was determined to be 0.45 hours for plasma, 22 hours for liver, 19 hours for kidney, and 24 hours for spleen, indicating rapid tissue clearance. Tissue half-life of Lipid #3 could not be determined for the lung and heart.

### Further Considerations

The foregoing description is provided to enable a person skilled in the art to practice the various configurations described herein. There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

SEQUENCE LISTING

mRNA coding sequence for wild type human OTC (SEQ ID NO: 1)
AUGCUGUUUAAUCUGAGGAUCCUGUUAAACAAUGCAGCUUUUAGAAAUGGUCAC
AACUUCAUGGUUCGAAAUUUUCGGUGUGGACAACCACUACAAAAUAAAGUGCAG
CUGAAGGGCCGUGACCUUCUCACUCUAAAAAAC
UUUACCGGAGAAGAAAUUAAAUAUAUGCUAUGGCUAUCAGCAGAUCUGAAAUUU
AGGAUAAAACAGAAAGGAGAGUAUUUGCCUUUAUUGCAAGGGAAGUCCUUAGGC
AUGAUUUUUGAGAAAAGAAGUACUCGAACAAGAUUGUCUACAGAAACAGGCUUU
GCACUUCUGGGAGGACAUCCUUGUUUUCUUACCACACAAGAUAUUCAUUUGGGU
GUGAAUGAAAGUCUCACGGACACGGCCCGUGUAUUGUCUAGCAUGGCAGAUGCA
GUAUUGGCUCGAGUGUAUAAACAAUCAGAUUUGGACACCCUGGCUAAAGAAGCA
UCCAUCCCAAUUAUCAAUGGGCUGUCAGAUUUGUACCAUCCUAUCCAGAUCCUG
GCUGAUUACCUCACGCUCCAGGAACACUAUAGCUCUCUGAAAGGUCUUACCCUCA
GCUGGAUCGGGGAUGGGAACAAUAUCCUGCACUCCAUCAUGAUGAGCGCAGCGA
AAUUCGGAAUGCACCUUCAGGCAGCUACUCCAAAGGGUUAUGAGCCGGAUGCUA
GUGUAACCAAGUUGGCAGAGCAGUAUGCCAAAGAGAAUGGUACCAAGCUGUUGC
UGACAAAUGAUCCAUUGGAAGCAGCGCAUGGAGGCAAUGUAUUAAUUACAGACA
CUUGGAUAAGCAUGGGACAAGAAGAGGAGAAGAAAAAGCGGCUCCAGGCUUUCC
AAGGUUACCAGGUUACAAUGAAGACUGCUAAAGUUGCUGCCUCUGACUGGACAU
UUUUACACUGCUUGCCCAGAAAGCCAGAAGAAGUGGAUGAUGAAGUCUUUUAUU
CUCCUCGAUCACUAGUGUUCCCAGAGGCAGAAAACAGAAAGUGGACAAUCAUGG
CUGUCAUGGUGUCCCUGCUGACAGAUUACUCACCUCAGCUCCAGAAGCCUAAAUU
UUGA
DNA coding sequence for wild type human OTC (SEQ ID NO: 2)
ATGCTGTTTAATCTGAGGATCCTGTTAAACAATGCAGCTTTTAGAAATGGTCACAAC
TTCATGGTTCGAAATTTTCGGTGTGGACAACCACTACAAAATAAAGTGCAGCTGAA
GGGCCGTGACCTTCTCACTCTAAAAAACTTTACCGGAGAAGAAATTAAATATATGC
TATGGCTATCAGCAGATCTGAAATTTAGGATAAAACAGAAAGGAGAGTATTTGCCT
TTATTGCAAGGGAAGTCCTTAGGCATGATTTTTGAGAAAAGAAGTACTCGAACAAG
ATTGTCTACAGAAACAGGCTTTGCACTTCTGGGAGGACATCCTTGTTTTCTTACCAC
ACAAGATATTCATTTGGGTGTGAATGAAAGTCTCACGGACACGGCCCGTGTATTGTC
TAGCATGGCAGATGCAGTATTGGCTCGAGTGTATAAACAATCAGATTTGGACACCC
TGGCTAAAGAAGCATCCATCCCAATTATCAATGGGCTGTCAGATTTGTACCATCCTA
TCCAGATCCTGGCTGATTACCTCACGCTCCAGGAACACTATAGCTCTCTGAAAGGTC
TTACCCTCAGCTGGATCGGGGATGGGAACAATATCCTGCACTCCATCATGATGAGC
GCAGCGAAATTCGGAATGCACCTTCAGGCAGCTACTCCAAAGGGTTATGAGCCGGA
TGCTAGTGTAACCAAGTTGGCAGAGCAGTATGCCAAAGAGAATGGTACCAAGCTGT
TGCTGACAAATGATCCATTGGAAGCAGCGCATGGAGGCAATGTATTAATTACAGAC
ACTTGGATAAGCATGGGACAAGAAGAGGAGAAGAAAAAGCGGCTCCAGGCTTTCC
AAGGTTACCAGGTTACAATGAAGACTGCTAAAGTTGCTGCCTCTGACTGGACATTTT
TACACTGCTTGCCCAGAAAGCCAGAAGAAGTGGATGATGAAGTCTTTTATTCTCCTC
GATCACTAGTGTTCCCAGAGGCAGAAAACAGAAAGTGGACAATCATGGCTGTCATG
GTGTCCCTGCTGACAGATTACTCACCTCAGCTCCAGAAGCCTAAATTTTGA
Human wild type OTC amino acid sequence
(SEQ ID NO: 3)
MLFNLRILLNNAAFRNGHNFMVRNFRCGQPLQNKVQLKGRDLLTLKNFTGEEIKYML
WLSADLKFRIKQKGEYLPLLQGKSLGMIFEKRSTRTRLSTETGFALLGGHPCFLTTQDIH
LGVNESLTDTARVLSSMADAVLARVYKQSDLDTLAKEASIPIINGLSDLYHPIQILADYL
TLQEHYSSLKGLTLSWIGDGNNILHSIMMSAAKFGMHLQAATPKGYEPDASVTKLAEQ
YAKENGTKLLLTNDPLEAAHGGNVLITDTWISMGQEEEKKKRLQAFQGYQVTMKTAK
VAASDWTFLHCLPRKPEEVDDEVFYSPRSLVFPEAENRKWTIMAVMVSLLTDYSPQLQ
KPKF
Modified OTC amino acid sequence
(SEQ ID NO: 4)
MLVFNLRILLNNAAFRNGHNFMVRNFRCGQPLQNRVQLKGRDLLTLKNFTGEEIRYML
WLSADLKFRIKQKGEYLPLLQGKSLGMIFEKRSTRTRLSTETGFALLGGHPCFLTTQDIH
LGVNESLTDTARVLSSMADAVLARVYKQSDLDTLAKEASIPIINGLSDLYHPIQILADYL
TLQEHYSSLKGLTLSWIGDGNNILHSIMMSAAKFGMHLQAATPKGYEPDASVTKLAEQ
YAKENGTKLLLTNDPLEAAHGGNVLITDTWISMGQEEEKKKRLQAFQGYQVTMKTAK
VAASDWTFLHCLPRKPEEVDDEVFYSPRSLVFPEAENRKWTIMAVMVSLLTDYSPQLQ
KPKF
TEV (SEQ ID NO: 5)
TCAACACAACATATACAAAACAAACGAATCTCAAGCAATCAAGCATTCTACTTCTA
TTGCAGCAATTTAAATCATTTCTTTTAAAGCAAAAGCAATTTTCTGAAAATTTTCAC
CATTTACGAACGATAG
AT1G58420 (SEQ ID NO: 6)
ATTATTACATCAAAACAAAAAGCCGCCA
ARC5-2 (SEQ ID NO: 7)
CTTAAGGGGGCGCTGCCTACGGAGGTGGCAGCCATCTCCTTCTCGGCATCAAGCTTA
CCATGGTGCCCCAGGCCCTGCTCTTgGTCCCGCTGCTGGTGTTCCCCCTCTGCTTCGG
CAAGTTCCCCATCTACACCATCCCCGACAAGCTGGGGCCGTGGAGCCCCATCGACA
TCCACCACCTGTCCTGCCCCAACAACCTCGTGGTCGAGGACGAGGGCTGCACCAAC
CTGAGCGGGTTCTCCTAC
HCV (SEQ ID NO: 8)
TGAGTGTCGT ACAGCCTCCA GGCCCCCCCC TCCCGGGAGA GCCATAGTGG
TCTGCGGAACCGGTGAGTAC ACCGGAATTG CCGGGAAGAC TGGGTCCTTT
CTTGGATAAA CCCACTCTATGCCCGGCCAT TTGGGCGTGC CCCCGCAAGA
CTGCTAGCCG AGTAGTGTTG GGTTGCG
HUMAN ALBUMIN (SEQ ID NO: 9)
AATTATTGGTTAAAGAAGTATATTAGTGCTAATTTCCCTCCGTTTGTCCTAGCTTTTC
TCTTCTGTCAACCCCACACGCCTTTGGCACA
EMCV (SEQ ID NO: 10)
CTCCCTCCCC CCCCCCTAAC GTTACTGGCC GAAGCCGCTT GGAATAAGGC
CGGTGTGCGT TTGTCTATAT GTTATTTTCC ACCATATTGC CGTCTTTTGG
CAATGTGAGG GCCCGGAAAC CTGGCCCTGT CTTCTTGACG AGCATTCCTA
GGGGTCTTTC CCCTCTCGCC AAAGGAATGC AAGGTCTGTT GAATGTCGTG
AAGGAAGCAG TTCCTCTGGA AGCTTCTTGA AGACAAACAA CGTCTGTAGC
GACCCTTTGC AGGCAGCGGA ACCCCCCACC TGGCGACAGG TGCCTCTGCG
GCCAAAAGCC ACGTGTATAA GATACACCTG CAAAGGCGGC ACAACCCCAG
TGCCACGTTG TGAGTTGGAT AGTTGTGGAA AGAGTCAAAT GGCTCTCCTC
AAGCGTATTC AACAAGGGGC TGAAGGATGC CCAGAAGGTA CCCCATTGTA
TGGGATCTGA TCTGGGGCCT CGGTGCACAT GCTTTACGTG TGTTTAGTCG
AGGTTAAAAA ACGTCTAGGC CCCCCGAACC ACGGGGACGT GGTTTTCCTT
TGAAAAACAC GATGATAAT
HSP70-P2 (SEQ ID NO: 11)
GTCAGCTTTCAAACTCTTTGTTTCTTGTTTGTTGATTGAGAATA
HSP70-M1 (SEQ ID NO: 12)
CTCTCGCCTGAGAAAAAAAATCCACGAACCAATTTCTCAGCAACCAGCAGCACG
HSP72-M2 (SEQ ID NO: 13)
ACCTGTGAGGGTTCGAAGGAAGTAGCAGTGTTTTTTGTTCCTAGAGGAAGAG
HSP17.9 (SEQ ID NO: 14)
ACACAGAAACATTCGCAAAAACAAAATCCCAGTATCAAAATTCTTCTCTTTTTTTCA
TATTTCGCAAAGAC
HSP70-P1 (SEQ ID NO: 15)
CAGAAAAATTTGCTACATTGTTTCACAAACTTCAAATATTATTCATTTATTT
XBG (SEQ ID NO: 16)
CTAGTGACTGACTAGGATCTGGTTACCACTAAACCAGCCTCAAGAACACCCGAATG
GAGTCTCTAAGCTACATAATACCAACTTACACTTACAAAATGTTGTCCCCCAAAATG
TAGCCATTCGTATCTGCTCCTAATAAAAAGAAAGTTTCTTCACAT
HUMAN HAPTOGLOBIN (SEQ ID NO: 17)
TGCAAGGCTGGCCGGAAGCCCTTGCCTGAAAGCAAGATTTCAGCCTGGAAGAGGGC
AAAGTGGACGGGAGTGGACAGGAGTGGATGCGATAAGATGTGGTTTGAAGCTGAT
GGGTGCCAGCCCTGCATTGCTGAGTCAATCAATAAAGAGCTTTCTTTTGACCCAT
HUMAN APOLIPOPROTEIN E (SEQ ID NO: 18)
ACGCCGAAGCCTGCAGCCATGCGACCCCACGCCACCCCGTGCCTCCTGCCTCCGCG
CAGCCTGCAGCGGGAGACCCTGTCCCCGCCCCAGCCGTCCTCCTGGGGTGGACCCT
AGTTTAATAAAGATTCACCAAGTTTCACGCA
HCV (SEQ ID NO: 19)
TAGAGCGGCAAACCCTAGCTACACTCCATAGCTAGTTTCTTTTTTTTTTGTTTTTTTT
TTTTTTTTTTTTTTTTTTTTTTTTTTTTTTTCCTTTCTTTTCCTTCTTTTTTTCCTCTTTTC
TTGGTGGCTCCATCTTAGCCCTAGTCACGGCTAGCTGTGAAAGGTCCGTGAGCCGCA
TGACTGCAGAGAGTGCCGTAACTGGTCTCTCTGCAGATCATGT
MOUSE ALBUMIN (SEQ ID NO: 20)
ACACATCACAACCACAACCTTCTCAGGCTACCCTGAGAAAAAAAGACATGAAGACT
CAGGACTCATCTTTTCTGTTGGTGTAAAATCAACACCCTAAGGAACACAAATTTCTT
TAAACATTTGACTTCTTGTCTCTGTGCTGCAATTAATAAAAAATGGAAAGAATCTAC
HUMAN ALPHA GLOBIN (SEQ ID NO: 21)
GCTGGAGCCTCGGTAGCCGTTCCTCCTGCCCGCTGGGCCTCCCAACGGGCCCTCCTC
CCCTCCTTGCACCGGCCCTTCCTGGTCTTTGAATAAAGTCTGAGTGGGCAGCA
EMCV (SEQ ID NO: 22)
TAGTGCAGTCAC TGGCACAACG CGTTGCCCGG TAAGCCAATC GGGTATACAC
GGTCGTCATACTGCAGACAG GGTTCTTCTA CTTTGCAAGA TAGTCTAGAG
TAGTAAAATA AATAGTATAAG
(SEQ ID NO: 23)
GCCACC
(SEQ ID NO: 24)
GCCA
(SEQ ID NO: 25)
AUAAGUGAA
>mARM563 (SEQ ID NO: 26)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUGUUUAAUCUGAGGAUCCUGUUAA
ACAAUGCAGCUUUUAGAAAUGGUCACAACUUCAUGGUUCGAAAUUUUCGGUGUG
GACAACCACUACAAAAUAAAGUGCAGCUGAAGGGCCGUGACCUUCUCACUCUAA
AAAACUUUACCGGAGAAGAAAUUAAAUAUAUGCUAUGGCUAUCAGCAGAUCUGA
AAUUUAGGAUAAAACAGAAAGGAGAGUAUUUGCCUUUAUUGCAAGGGAAGUCCU
UAGGCAUGAUUUUUGAGAAAAGAAGUACUCGAACAAGAUUGUCUACAGAAACAG
GCUUUGCACUUCUGGGAGGACAUCCUUGUUUUCUUACCACACAAGAUAUUCAUU
UGGGUGUGAAUGAAAGUCUCACGGACACGGCCCGUGUAUUGUCUAGCAUGGCAG
AUGCAGUAUUGGCUCGAGUGUAUAAACAAUCAGAUUUGGACACCCUGGCUAAAG
AAGCAUCCAUCCCAAUUAUCAAUGGGCUGUCAGAUUUGUACCAUCCUAUCCAGA
UCCUGGCUGAUUACCUCACGCUCCAGGAACACUAUAGCUCUCUGAAAGGUCUUAC
CCUCAGCUGGAUCGGGGAUGGGAACAAUAUCCUGCACUCCAUCAUGAUGAGCGC
AGCGAAAUUCGGAAUGCACCUUCAGGCAGCUACUCCAAAGGGUUAUGAGCCGGA
UGCUAGUGUAACCAAGUUGGCAGAGCAGUAUGCCAAAGAGAAUGGUACCAAGCU
GUUGCUGACAAAUGAUCCAUUGGAAGCAGCGCAUGGAGGCAAUGUAUUAAUUAC
AGACACUUGGAUAAGCAUGGGACAAGAAGAGGAGAAGAAAAAGCGGCUCCAGGC
UUUCCAAGGUUACCAGGUUACAAUGAAGACUGCUAAAGUUGCUGCCUCUGACUG
GACAUUUUUACACUGCUUGCCCAGAAAGCCAGAAGAAGUGGAUGAUGAAGUCUU
UUAUUCUCCUCGAUCACUAGUGUUCCCAGAGGCAGAAAACAGAAAGUGGACAAU
CAUGGCUGUCAUGGUGUCCCUGCUGACAGAUUACUCACCUCAGCUCCAGAAGCCU
AAAUUUUGACUCGAGCUAGUGACUGACUAGGAUCUGGUUACCACUAAACCAGCC
UCAAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAAAG
UUUCUUCACAUUCUAG
>mARM564 (SEQ ID NO: 27)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUCUUUAAUCUGCGCAUCUUACUGA
ACAACGCCGCAUUCCGGAACGGUCACAACUUCAUGGUCCGCAAUUUCCGCUGUGG
CCAGCCGCUUCAAAACAAGGUCCAGCUGAAGGGACGGGAUCUGCUGACACUGAA
GAACUUCACCGGAGAAGAGAUCAAGUACAUGCUGUGGCUCAGCGCAGACUUGAA
GUUCCGGAUCAAGCAGAAGGGAGAAUACUUGCCCCUGCUGCAAGGAAAGUCGCU
GGGAAUGAUUUUUGAGAAGCGGUCAACUCGCACCAGACUCUCCACCGAAACUGG
UUUCGCACUGCUUGGCGGGCACCCUUGCUUCCUGACGACUCAGGACAUCCACCUC
GGCGUGAACGAAUCGCUAACCGAUACCGCCAGAGUGCUUUCUUCCAUGGCCGACG
CGGUGCUGGCCAGGGUGUACAAGCAGUCCGACCUCGAUACCUUGGCAAAGGAGG
CUUCCAUUCCCAUCAUCAACGGCCUGAGCGACCUGUACCACCCAAUCCAAAUCCU
GGCUGACUACCUGACCCUGCAAGAGCACUACAGCAGCCUGAAGGGUCUGACCCUG
UCAUGGAUUGGCGAUGGAAACAAUAUUCUGCACUCCAUCAUGAUGUCCGCCGCG
AAGUUCGGAAUGCAUCUGCAAGCCGCCACUCCAAAAGGAUACGAACCGGAUGCG
UCCGUGACCAAGUUGGCGGAACAGUACGCGAAGGAGAACGGAACCAAGCUUCUG
CUGACUAACGACCCCCUCGAGGCUGCGCAUGGGGGCAACGUGCUGAUUACCGACA
CCUGGAUCUCCAUGGGGCAGGAGGAAGAGAAGAAGAAGAGACUGCAGGCAUUCC
AGGGGUACCAGGUCACCAUGAAAACCGCAAAAGUGGCAGCUUCGGACUGGACUU
UCCUGCAUUGCCUGCCGAGGAAGCCGGAGGAAGUCGACGACGAAGUGUUCUACU
CGCCUCGGUCCCUGGUGUUCCCCGAGGCCGAAAACCGGAAGUGGACCAUCAUGGC
CGUGAUGGUGUCCUUGCUGACUGACUAUAGCCCGCAGCUGCAGAAGCCUAAGUU
CUAGCUCGAGCUAGUGACUGACUAGGAUCUGGUUACCACUAAACCAGCCUCAAG
AACACCCGAAUGGAGUCUCUAAGCUACAUAAUACCAACUUACACUUACAAAAUG
UUGUCCCCCAAAAUGUAGCCAUUCGUAUCUGCUCCUAAUAAAAAGAAAGUUUCU
UCACAUUCUAG
>mARM565 (SEQ ID NO: 28)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUGUUUAACCUACGUAUUUUGCUCA
ACAAUGCAGCCUUUAGAAACGGACAUAACUUUAUGGUUCGAAACUUUCGCUGCG
GGCAGCCACUGCAGAACAAGGUCCAGCUGAAAGGGAGAGAUUUGCUCACGCUGA
AGAACUUUACUGGCGAAGAAAUCAAGUAUAUGCUGUGGUUGUCCGCGGACCUCA
AGUUUCGGAUUAAGCAGAAAGGGGAGUAUCUGCCACUGCUGCAAGGAAAGAGCC
UCGGCAUGAUCUUCGAGAAGCGGAGCACUCGGACCAGGCUGAGUACCGAAACUG
GCUUCGCAUUGUUGGGUGGACAUCCAUGUUUUCUGACAACGCAGGACAUUCAUC
UGGGCGUGAACGAGAGUCUGACGGACACAGCUCGCGUUCUGUCCUCUAUGGCUG
AUGCGGUGUUGGCCCGGGUCUAUAAGCAGUCCGAUUUGGACACCUUGGCUAAGG
AAGCUAGCAUACCGAUUAUCAAUGGGCUGUCCGACCUGUAUCACCCUAUUCAAA
UCCUGGCCGACUACCUCACACUGCAAGAACACUAUAGCUCAUUGAAGGGACUGAC
CCUGAGCUGGAUAGGGGACGGAAACAACAUCCUACAUAGCAUUAUGAUGUCCGC
UGCCAAGUUUGGCAUGCAUCUUCAAGCCGCCACGCCAAAGGGUUAUGAGCCCGAC
GCGUCAGUGACAAAGCUGGCCGAGCAGUACGCUAAGGAGAAUGGUACCAAAUUA
CUGCUGACUAAUGAUCCACUGGAGGCUGCACAUGGCGGCAAUGUACUGAUCACC
GACACAUGGAUCUCGAUGGGCCAGGAGGAAGAAAAGAAGAAGAGGCUUCAGGCC
UUCCAAGGCUACCAGGUCACCAUGAAAACAGCUAAGGUUGCAGCAUCUGAUUGG
ACCUUUCUGCACUGUCUGCCAAGGAAGCCCGAAGAGGUGGACGAUGAAGUAUUC
UAUAGCCCACGGAGUUUGGUGUUCCCUGAGGCUGAAAAUAGGAAGUGGACAAUU
AUGGCCGUAAUGGUGUCCCUGUUAACCGACUACUCUCCGCAACUGCAGAAACCUA
AGUUUUAGCUCGAGCUAGUGACUGACUAGGAUCUGGUUACCACUAAACCAGCCU
CAAGAACACCCGAAUGGAGUCUCUAAGCUACAUAAUACCAACUUACACUUACAA
AAUGUUGUCCCCCAAAAUGUAGCCAUUCGUAUCUGCUCCUAAUAAAAAGAAAGU
UUCUUCACAUUCUAG
>mARM566 (SEQ ID NO: 29)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUGUUUAACUUAAGGAUCCUGCUGA
ACAACGCCGCUUUUCGUAACGGUCAUAACUUUAUGGUCCGGAACUUUAGAUGUG
GCCAGCCGCUGCAGAACAAGGUUCAGCUGAAGGGGAGGGAUCUGCUGACCUUGA
AGAACUUUACCGGCGAAGAGAUCAAGUACAUGUUGUGGCUGAGCGCCGAUCUGA
AGUUUAGGAUUAAGCAGAAGGGGGAGUAUUUGCCACUGCUGCAAGGAAAAUCCU
UGGGGAUGAUCUUCGAGAAGCGCUCCACUAGAACCCGGCUAAGCACAGAAACCG
GCUUCGCACUUCUGGGUGGACAUCCCUGUUUUCUGACGACGCAGGAUAUACACC
UGGGCGUGAAUGAGAGUCUGACGGACACAGCUAGGGUGUUGAGCAGCAUGGCCG
AUGCAGUACUGGCCCGCGUUUAUAAGCAGAGCGACUUGGACACACUGGCCAAGG
AAGCGUCAAUUCCGAUUAUCAAUGGGCUGUCAGACCUGUAUCAUCCCAUUCAAA
UCUUGGCUGACUAUCUGACCCUGCAAGAACAUUACAGCUCCCUGAAGGGCCUCAC
GUUGUCCUGGAUUGGCGACGGAAACAACAUUCUGCAUUCGAUCAUGAUGAGCGC
UGCUAAGUUUGGCAUGCACCUCCAAGCCGCUACACCUAAGGGAUAUGAGCCUGA
UGCCAGCGUAACCAAGCUGGCCGAACAGUACGCGAAGGAGAAUGGCACGAAACU
GCUGUUGACAAAUGACCCACUGGAGGCAGCUCACGGUGGCAACGUGCUGAUCAC
CGACACGUGGAUAUCUAUGGGACAGGAAGAAGAGAAGAAGAAGCGGCUGCAGGC
AUUCCAAGGGUAUCAGGUCACCAUGAAAACGGCCAAGGUUGCUGCAUCCGACUG
GACAUUUCUGCAUUGCUUGCCCCGCAAACCAGAAGAAGUAGACGACGAAGUCUU
UUAUUCCCCACGGUCGCUGGUGUUCCCCGAGGCGGAGAAUCGAAAGUGGACGAU
UAUGGCCGUGAUGGUGUCCCUGCUGACUGAUUACUCUCCCCAACUGCAAAAGCCU
AAGUUUUAGCUCGAGCUAGUGACUGACUAGGAUCUGGUUACCACUAAACCAGCC
UCAAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAAAG
UUUCUUCACAUUCUAG
>mARM567 (SEQ ID NO: 30)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAACCUGAGGAUCCUCCUGAA
CAACGCCGCCUUUCGCAAUGGUCACAACUUUAUGGUCCGGAACUUCAGAUGCGGC
CAGCCGCUGCAGAACAAGGUCCAGCUGAAGGGACGGGAUCUGCUGACUCUGAAG
AACUUCACCGGAGAAGAGAUCAAGUACAUGCUGUGGCUGUCGGCCGACCUGAAG
UUCAGGAUCAAGCAGAAGGGAGAAUACCUCCCGCUGCUGCAAGGAAAGUCCCUG
GGCAUGAUUUUCGAGAAGCGCUCGACCAGAACUCGGUUGUCCACCGAAACCGGG
UUUGCGCUGCUGGGCGGACAUCCUUGCUUCCUGACGACUCAGGAUAUUCACCUG
GGAGUGAACGAGUCGCUGACCGACACCGCCAGAGUGCUGAGCUCGAUGGCCGAC
GCCGUGUUGGCACGCGUGUACAAGCAGUCCGAUCUGGAUACCCUGGCCAAAGAA
GCUUCCAUCCCGAUCAUUAACGGGCUGAGCGACCUCUACCACCCCAUUCAAAUCC
UGGCCGACUACCUGACUCUGCAAGAACACUACAGCUCGCUGAAGGGGUUGACUC
UGUCCUGGAUCGGCGACGGAAACAACAUCCUGCACUCCAUCAUGAUGUCGGCCGC
AAAGUUCGGCAUGCAUUUGCAAGCCGCCACCCCAAAGGGCUACGAACCAGACGCG
AGCGUCACCAAGCUGGCCGAACAGUACGCGAAGGAAAAUGGUACUAAGCUGCUG
CUGACCAACGACCCAUUGGAAGCUGCCCAUGGUGGAAACGUGCUGAUCACCGACA
CCUGGAUCUCGAUGGGCCAGGAAGAGGAGAAGAAGAAGCGGCUGCAGGCGUUCC
AGGGGUAUCAGGUCACCAUGAAAACAGCCAAAGUGGCAGCGUCAGACUGGACCU
UCCUCCACUGUCUGCCUCGCAAGCCAGAGGAGGUGGACGACGAGGUGUUCUACUC
CCCUCGGUCCCUCGUGUUCCCUGAGGCUGAGAACCGGAAGUGGACCAUUAUGGCC
GUGAUGGUGUCACUCCUGACUGAUUACUCCCCGCAACUGCAGAAGCCCAAGUUCU
AGCUCGAGCUAGUGACUGACUAGGAUCUGGUUACCACUAAACCAGCCUCAAGAA
CACCCGAAUGGAGUCUCUAAGCUACAUAAUACCAACUUACACUUACAAAAUGUU
GUCCCCCAAAAUGUAGCCAUUCGUAUCUGCUCCUAAUAAAAAGAAAGUUUCUUC
ACAUUCUAG
>mARM568 (SEQ ID NO: 31)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUGUUUAACCUGAGGAUCCUAUUGA
ACAAUGCUGCUUUUCGUAAUGGCCAUAACUUUAUGGUUCGGAACUUUAGAUGCG
GGCAGCCACUGCAGAACAAGGUCCAGUUGAAAGGCCGCGAUCUGUUGACAUUGA
AGAACUUUACCGGCGAAGAGAUUAAGUAUAUGCUGUGGCUGUCUGCUGACCUCA
AGUUUCGAAUCAAGCAGAAGGGCGAAUAUCUCCCCCUGCUGCAAGGAAAGUCUC
UCGGCAUGAUCUUUGAGAAGCGGAGUACCCGAACACGGCUGAGCACCGAAACGG
GCUUCGCACUGCUGGGGGGCCAUCCCUGUUUUCUGACAACGCAGGACAUCCACUU
GGGGGUUAACGAAUCAUUGACUGAUACCGCCCGCGUACUGUCAUCCAUGGCCGA
CGCUGUGCUGGCUAGGGUGUACAAGCAGUCAGAUCUGGAUACACUGGCCAAGGA
AGCUAGCAUACCAAUCAUCAAUGGACUGAGUGACCUUUAUCACCCGAUUCAAAU
ACUAGCCGAUUAUCUGACCCUGCAAGAGCAUUACUCCUCGCUGAAAGGCCUCACG
CUGUCCUGGAUCGGCGACGGCAACAACAUUCUGCAUAGUAUUAUGAUGUCUGCU
GCCAAAUUCGGCAUGCAUCUGCAAGCUGCUACGCCGAAGGGUUAUGAACCCGAC
GCGUCAGUUACGAAGCUCGCUGAGCAGUAUGCAAAGGAGAAUGGCACAAAGCUG
UUGCUUACCAACGAUCCCCUGGAAGCUGCUCAUGGCGGCAAUGUGCUGAUUACU
GACACCUGGAUUUCAAUGGGCCAGGAGGAGGAGAAGAAGAAGAGGUUACAGGCU
UUUCAAGGUUACCAAGUCACGAUGAAAACCGCUAAGGUCGCAGCCAGCGACUGG
ACAUUCCUGCACUGUCUGCCAAGAAAGCCGGAAGAAGUGGACGACGAGGUGUUC
UAUUCCCCGCGGUCUUUGGUGUUUCCGGAGGCCGAAAACAGGAAAUGGACCAUU
AUGGCCGUGAUGGUAUCGUUGCUGACGGACUACAGCCCUCAGUUGCAAAAGCCC
AAGUUCUAGCUCGAGCUAGUGACUGACUAGGAUCUGGUUACCACUAAACCAGCC
UCAAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAAAG
UUUCUUCACAUUCUAG
>mARM569 (SEQ ID NO: 32)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUCUUUAACCUCCGCAUCCUCCUCAA
CAACGCCGCCUUCCGGAAUGGGCAUAACUUCAUGGUCCGGAACUUCAGAUGCGGC
CAGCCCCUGCAAAACAAGGUCCAGUUGAAGGGACGGGACCUCCUUACGCUGAAG
AACUUUACCGGAGAAGAGAUUAAGUACAUGCUGUGGUUGUCCGCUGACCUCAAG
UUCCGCAUUAAGCAGAAGGGAGAAUAUCUGCCGCUGCUGCAAGGAAAGAGCCUG
GGCAUGAUCUUCGAAAAGCGCUCCACUAGAACCCGGCUGUCGACUGAGACUGGA
UUCGCCUUGCUCGGUGGACACCCGUGCUUCCUGACGACCCAGGACAUCCACCUGG
GAGUGAACGAGUCACUUACGGAUACCGCGAGGGUGCUGUCCUCAAUGGCCGACG
CAGUGCUCGCGCGCGUGUACAAGCAGUCAGAUCUGGAUACCCUGGCCAAGGAAG
CCAGCAUUCCCAUCAUCAACGGACUGAGCGACCUUUACCACCCAAUCCAGAUCCU
CGCCGACUACUUAACCCUGCAAGAGCACUACAGCUCCCUGAAGGGACUGACUCUG
UCCUGGAUCGGGGAUGGAAACAACAUCCUGCACUCCAUCAUGAUGUCUGCCGCU
AAGUUUGGGAUGCAUCUGCAAGCCGCAACCCCUAAGGGAUACGAGCCCGACGCCU
CGGUGACCAAACUUGCGGAACAGUACGCCAAGGAAAACGGUACCAAGCUGCUGC
UGACCAACGACCCUCUGGAAGCGGCCCACGGAGGAAAUGUGCUGAUUACCGACAC
CUGGAUUUCGAUGGGCCAGGAGGAGGAGAAGAAGAAGAGACUGCAGGCGUUCCA
GGGAUAUCAGGUCACCAUGAAAACCGCCAAGGUCGCCGCCAGCGACUGGACCUUC
CUGCACUGUCUCCCUCGGAAACCGGAAGAAGUGGAUGACGAGGUGUUCUACUCC
CCGCGCUCGCUGGUGUUCCCGGAGGCUGAAAACAGGAAGUGGACAAUCAUGGCC
GUGAUGGUGUCCCUGUUGACCGACUACUCCCCACAACUGCAGAAGCCCAAGUUCU
AGCUCGAGCUAGUGACUGACUAGGAUCUGGUUACCACUAAACCAGCCUCAAGAA
CACCCGAAUGGAGUCUCUAAGCUACAUAAUACCAACUUACACUUACAAAAUGUU
GUCCCCCAAAAUGUAGCCAUUCGUAUCUGCUCCUAAUAAAAAGAAAGUUUCUUC
ACAUUCUAG
>mARM570 (SEQ ID NO: 33)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAAUCUGCGCAUCCUCCUGAA
CAACGCCGCCUUCCGCAAUGGACACAACUUUAUGGUCCGCAACUUCCGCUGUGGG
CAGCCGCUGCAGAACAAGGUCCAGCUCAAGGGGAGAGAUCUCCUGACCCUGAAG
AACUUCACUGGAGAGGAGAUCAAGUACAUGCUGUGGCUGUCCGCCGACCUGAAA
UUUCGGAUUAAGCAGAAGGGCGAAUACCUCCCACUGCUGCAAGGAAAGUCUUUG
GGCAUGAUCUUCGAAAAGAGAAGCACCCGGACCCGGUUGAGCACCGAAACUGGG
UUCGCGCUCCUCGGUGGACACCCGUGCUUCCUGACCACCCAAGAUAUUCAUCUGG
GUGUCAACGAAAGCCUGACCGACACCGCCAGGGUGCUGUCAUCCAUGGCUGACGC
AGUGCUCGCCCGGGUGUACAAGCAGUCAGACCUGGACACCCUCGCCAAGGAAGCU
UCGAUCCCUAUCAUCAACGGACUUUCCGACCUGUACCACCCCAUCCAAAUUCUGG
CCGACUACCUGACUCUGCAAGAACACUAUAGCUCGCUGAAAGGACUUACUCUGUC
CUGGAUCGGGGACGGCAACAACAUUCUCCAUUCCAUCAUGAUGUCCGCUGCCAAG
UUCGGAAUGCACCUUCAAGCAGCGACUCCCAAGGGAUACGAACCUGAUGCCUCCG
UGACUAAGCUGGCAGAGCAGUACGCCAAGGAGAACGGUACAAAGCUGCUGCUCA
CGAACGACCCCCUGGAGGCGGCCCACGGCGGAAACGUGCUGAUUACCGAUACCUG
GAUCUCAAUGGGCCAGGAAGAGGAGAAGAAGAAGCGGCUCCAGGCGUUUCAAGG
CUACCAGGUCACCAUGAAAACCGCGAAGGUCGCCGCCUCCGACUGGACUUUCUUG
CACUGCCUGCCGCGGAAGCCCGAGGAAGUGGAUGACGAAGUGUUCUACUCGCCG
AGAUCGUUGGUGUUCCCUGAGGCCGAAAACAGGAAGUGGACCAUCAUGGCCGUG
AUGGUGUCCCUGCUGACUGAUUACAGCCCACAGCUGCAGAAGCCUAAGUUCUAG
CUCGAGCUAGUGACUGACUAGGAUCUGGUUACCACUAAACCAGCCUCAAGAACA
CCCGAAUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUCUUCAC
AUUCUAG
>mARM571 (SEQ ID NO: 34)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAAUCUCCGCAUCCUCCUUAA
CAACGCCGCGUUUAGAAACGGCCACAACUUCAUGGUCCGGAACUUCAGAUGUGG
CCAGCCGCUUCAAAACAAGGUCCAGCUGAAGGGCCGGGAUCUUCUGACCCUGAAG
AACUUUACUGGCGAAGAGAUCAAGUACAUGCUCUGGCUCUCCGCGGACUUGAAG
UUCCGCAUUAAGCAGAAGGGGGAAUACCUUCCGCUGCUUCAAGGAAAGAGCCUC
GGCAUGAUCUUUGAGAAGCGCUCAACCAGGACCCGCCUUUCUACUGAAACUGGG
UUCGCGCUGCUCGGUGGCCACCCCUGCUUCCUGACGACCCAGGACAUCCACCUCG
GAGUGAACGAAUCCCUCACCGAUACCGCCCGGGUGUUAUCGAGCAUGGCAGAUG
CCGUGCUGGCCAGGGUGUACAAACAGUCCGAUCUGGACACUCUGGCCAAGGAGG
CGUCAAUUCCUAUUAUCAACGGCCUUAGUGACCUCUACCAUCCGAUUCAGAUCCU
GGCCGAUUACCUCACCCUGCAAGAACACUACAGCUCCCUGAAGGGUCUGACAUUG
UCCUGGAUCGGCGACGGCAACAACAUUCUCCAUUCCAUCAUGAUGUCCGCCGCAA
AAUUCGGCAUGCAUCUUCAAGCCGCCACGCCGAAGGGUUACGAGCCCGACGCUUC
CGUGACUAAGCUCGCCGAGCAGUACGCUAAGGAGAACGGAACCAAGCUUCUGCU
GACUAACGACCCACUAGAAGCAGCCCACGGGGGCAACGUGCUUAUUACUGACACC
UGGAUCUCCAUGGGCCAGGAAGAAGAGAAAAAGAAGCGGCUGCAGGCGUUCCAG
GGAUAUCAGGUCACCAUGAAAACCGCCAAGGUCGCUGCCUCCGACUGGACCUUCC
UGCACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCCGU
GAUGGUGUCACUGCUCACCGACUACAGCCCGCAGCUUCAGAAGCCCAAGUUCUAG
CUCGAGCUAGUGACUGACUAGGAUCUGGUUACCACUAAACCAGCCUCAAGAACA
CCCGAAUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUCUUCAC
AUUCUAG
>mARM572 (SEQ ID NO: 35)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAACCUGAGAAUCCUCCUGAA
CAACGCCGCCUUCCGCAAUGGUCAUAACUUCAUGGUCCGCAACUUUCGCUGCGGA
CAGCCUCUCCAAAACAAGGUCCAGCUCAAGGGGCGCGACCUCCUCACACUGAAGA
ACUUCACUGGAGAAGAAAUCAAGUACAUGCUGUGGCUGAGCGCCGAUCUGAAGU
UCCGGAUCAAGCAGAAGGGAGAGUACCUUCCUCUGCUGCAAGGGAAGUCCUUGG
GAAUGAUUUUCGAGAAGCGGUCCACCCGGACCAGGCUGAGCACUGAAACUGGCU
UCGCCCUGCUGGGAGGCCACCCUUGUUUCCUGACCACUCAGGACAUCCACCUGGG
CGUGAACGAGUCCCUGACCGAUACUGCCAGAGUGCUGUCCUCCAUGGCCGACGCC
GUGCUCGCCCGGGUGUACAAGCAGUCAGACCUCGAUACGCUGGCCAAGGAAGCCU
CCAUUCCCAUUAUCAAUGGUCUGUCGGACCUCUACCAUCCAAUCCAAAUCCUCGC
CGACUACCUGACUCUGCAAGAACACUACAGCUCACUCAAGGGCCUCACCCUCUCC
UGGAUCGGCGACGGAAACAACAUCCUUCACUCGAUUAUGAUGUCGGCCGCGAAG
UUCGGGAUGCACCUCCAAGCUGCCACUCCAAAAGGCUACGAGCCGGAUGCCUCAG
UGACUAAGUUGGCGGAACAGUAUGCGAAGGAGAACGGUACCAAGCUCCUGCUGA
CUAACGACCCGCUGGAGGCCGCCCACGGGGGAAACGUGCUCAUCACCGAUACUUG
GAUUUCCAUGGGACAGGAGGAAGAGAAGAAGAAGCGGUUGCAGGCAUUUCAGGG
CUACCAGGUCACCAUGAAAACUGCCAAAGUCGCCGCCAGCGACUGGACCUUCCUG
CACUGCCUGCCCCGCAAGCCUGAAGAAGUGGACGACGAGGUGUUCUACUCUCCCC
GGUCCCUCGUGUUCCCUGAGGCCGAAAACAGGAAGUGGACCAUCAUGGCUGUGA
UGGUGUCCCUCCUGACCGACUACAGCCCUCAGCUCCAAAAACCCAAGUUUUAGCU
CGAGCUAGUGACUGACUAGGAUCUGGUUACCACUAAACCAGCCUCAAGAACACCC
GAAUGGAGUCUCUAAGCUACAUAAUACCAACUUACACUUACAAAAUGUUGUCCC
CCAAAAUGUAGCCAUUCGUAUCUGCUCCUAAUAAAAAGAAAGUUUCUUCACAUU
CUAG
>mARM573 (SEQ ID NO: 36)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAACCUGAGAAUCCUCUUGA
ACAAUGCUGCUUUUCGGAAUGGCCACAACUUUAUGGUUCGGAACUUCCGUUGCG
GCCAGCCUUUACAAAACAAGGUCCAGCUGAAGGGCCGGGAUUUGCUCACACUGA
AGAACUUUACUGGGGAGGAGAUUAAGUAUAUGCUGUGGCUGUCCGCUGACCUGA
AGUUUAGGAUCAAGCAGAAGGGCGAAUAUCUGCCGCUGCUGCAAGGGAAAAGUC
UGGGCAUGAUUUUUGAAAAGCGCUCUACCCGGACCAGACUGUCUACGGAAACAG
GCUUUGCCCUGCUGGGCGGCCACCCCUGUUUUCUGACAACGCAGGACAUCCAUCU
GGGCGUGAACGAAUCACUGACCGAUACUGCUCGGGUACUCAGUUCUAUGGCUGA
CGCAGUGCUGGCUAGGGUGUACAAGCAGAGCGACUUGGACACACUGGCUAAGGA
GGCCAGCAUCCCCAUUAUCAAUGGCCUGUCUGAUUUGUACCAUCCCAUUCAAAUC
CUGGCUGAUUAUCUGACACUACAAGAGCAUUACUCAAGUCUGAAGGGUUUGACU
CUCUCCUGGAUCGGCGACGGCAACAACAUUUUACAUUCCAUUAUGAUGAGUGCU
GCUAAGUUUGGCAUGCAUUUGCAAGCUGCUACCCCAAAGGGCUAUGAACCUGAC
GCUAGCGUAACCAAGUUGGCCGAACAGUAUGCUAAAGAGAAUGGCACCAAGCUG
CUCCUGACGAAUGACCCCCUGGAAGCUGCUCAUGGCGGAAACGUACUUAUAACU
GAUACAUGGAUUAGCAUGGGCCAGGAAGAGGAGAAGAAGAAGAGACUGCAGGCC
UUCCAAGGCUAUCAGGUCACCAUGAAAACUGCCAAGGUUGCAGCUAGCGACUGG
ACCUUCCUGCACUGUUUGCCGAGGAAACCCGAGGAGGUGGACGAUGAAGUCUUU
UAUUCUCCCCGCUCCUUGGUGUUUCCCGAGGCUGAAAAUCGAAAGUGGACGAUA
AUGGCAGUGAUGGUGUCCCUACUGACCGACUAUUCUCCACAACUGCAGAAGCCU
AAAUUCUAGCUCGAGCUAGUGACUGACUAGGAUCUGGUUACCACUAAACCAGCC
UCAAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAAAG
UUUCUUCACAUUCUAG
>mARM574 (SEQ ID NO: 37)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAAUCUGAGGAUCCUGCUGA
ACAACGCUGCUUUUCGCAACGGUCAUAACUUUAUGGUUCGCAAUUUUCGUUGUG
GCCAGCCGCUGCAGAACAAGGUUCAGCUGAAGGGCAGAGAUCUGCUGACUCUGA
AGAACUUCACUGGGGAAGAAAUCAAGUAUAUGUUAUGGCUGUCCGCGGAUCUGA
AAUUUCGAAUCAAGCAGAAGGGCGAAUAUCUUCCCCUGCUGCAAGGGAAAUCCU
UGGGCAUGAUUUUUGAGAAGAGGAGCACUAGGACUAGAUUGUCAACAGAAACAG
GCUUUGCUUUGUUGGGCGGACAUCCCUGCUUUCUGACGACACAGGAUAUCCACC
UCGGCGUAAACGAGUCCCUCACCGACACUGCUAGGGUACUGAGCAGCAUGGCCGA
CGCUGUGCUAGCCCGGGUUUACAAGCAGUCAGACCUGGACACCCUUGCCAAGGAA
GCUUCUAUUCCAAUUAUCAACGGCCUGAGUGACCUGUAUCACCCUAUUCAAAUA
CUCGCCGACUAUUUGACGCUUCAAGAACAUUACAGCAGCCUCAAGGGCUUAACCU
UGAGUUGGAUAGGCGACGGCAACAAUAUCCUGCAUUCCAUUAUGAUGUCUGCCG
CUAAGUUUGGCAUGCAUCUACAAGCCGCAACACCCAAGGGCUAUGAACCCGACGC
UAGCGUGACCAAGCUGGCCGAGCAGUAUGCUAAGGAAAAUGGCACAAAGCUCCU
UCUUACCAACGAUCCCCUGGAGGCUGCUCACGGCGGCAACGUGCUGAUUACCGAU
ACAUGGAUUAGCAUGGGCCAGGAGGAGGAGAAAAAGAAGCGGCUCCAGGCUUUU
CAAGGCUAUCAGGUCACCAUGAAAACUGCAAAGGUCGCUGCCUCCGACUGGACU
UUCCUGCAUUGUCUACCCCGCAAGCCUGAGGAAGUGGACGAUGAGGUGUUCUAC
UCCCCACGGAGUCUGGUGUUCCCGGAAGCAGAGAAUCGGAAGUGGACCAUCAUG
GCUGUCAUGGUGUCGCUCUUGACUGACUAUUCUCCCCAACUGCAAAAACCCAAGU
UUUAGCUCGAGCUAGUGACUGACUAGGAUCUGGUUACCACUAAACCAGCCUCAA
GAACACCCGAAUGGAGUCUCUAAGCUACAUAAUACCAACUUACACUUACAAAAU
GUUGUCCCCCAAAAUGUAGCCAUUCGUAUCUGCUCCUAAUAAAAAGAAAGUUUC
UUCACAUUCUAG
>mARM575 (SEQ ID NO: 38)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGUUAUUCAACCUUCGUAUCCUGCUAA
ACAAUGCUGCUUUUCGCAAUGGCCAUAACUUUAUGGUUCGCAACUUUAGAUGCG
GCCAGCCGCUGCAGAACAAGGUUCAGCUGAAGGGCCGGGACUUGCUGACGCUGA
AAAACUUUACCGGGGAAGAGAUUAAGUAUAUGCUGUGGCUAAGCGCUGAUCUGA
AGUUUAGGAUCAAGCAGAAGGGCGAAUAUCUGCCACUGCUGCAAGGGAAGAGUC
UUGGCAUGAUUUUUGAAAAGCGGUCUACCAGAACCCGGCUGUCGACCGAGACAG
GUUUUGCUCUGCUGGGGGGCCAUCCCUGUUUUCUGACAACUCAGGACAUUCACC
UGGGCGUGAAUGAGUCCCUGACCGAUACUGCUAGGGUGUUGAGUAGCAUGGCCG
ACGCUGUACUCGCUCGAGUGUAUAAGCAGUCUGAUCUGGACACUCUGGCUAAGG
AAGCUUCCAUUCCUAUUAUCAACGGCUUGAGCGACCUGUACCACCCCAUUCAAAU
CCUCGCUGAUUACUUGACUUUGCAAGAACAUUACAGCAGCUUGAAGGGCUUAAC
ACUGAGCUGGAUAGGCGACGGAAACAACAUCUUGCAUUCCAUAAUGAUGUCCGC
CGCUAAGUUCGGGAUGCACCUCCAAGCAGCCACACCCAAGGGCUAUGAACCGGAU
GCUUCCGUGACAAAACUGGCUGAGCAGUAUGCUAAGGAGAAUGGCACGAAACUG
CUGCUCACCAACGACCCAUUGGAAGCUGCACAUGGUGGCAACGUACUGAUCACUG
ACACUUGGAUCUCAAUGGGCCAGGAGGAAGAGAAGAAGAAAAGGCUGCAGGCAU
UUCAGGGAUACCAAGUCACUAUGAAAACUGCCAAGGUCGCUGCCUCCGACUGGA
CAUUCCUGCAUUGUCUGCCACGGAAGCCUGAGGAAGUCGAUGACGAAGUGUUCU
AUAGCCCACGAAGCUUGGUGUUUCCCGAGGCUGAGAAUAGGAAGUGGACCAUUA
UGGCUGUUAUGGUGUCCCUGCUCACCGACUAUUCCCCUCAACUGCAAAAACCCAA
GUUUUAGCUCGAGCUAGUGACUGACUAGGAUCUGGUUACCACUAAACCAGCCUC
AAGAACACCCGAAUGGAGUCUCUAAGCUACAUAAUACCAACUUACACUUACAAA
AUGUUGUCCCCCAAAAUGUAGCCAUUCGUAUCUGCUCCUAAUAAAAAGAAAGUU
UCUUCACAUUCUAG
>mARM708 (SEQ ID NO: 39)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCAUGCUUUUUAAUCUCCGCAUCCUCCUUAACAA
CGCCGCGUUUAGAAACGGCCACAACUUCAUGGUCCGGAACUUCAGAUGUGGCCA
GCCGCUUCAAAACAAGGUCCAGCUGAAGGGCCGGGAUCUUCUGACCCUGAAGAA
CUUUACUGGCGAAGAGAUCAAGUACAUGCUCUGGCUCUCCGCGGACUUGAAGUU
CCGCAUUAAGCAGAAGGGGGAAUACCUUCCGCUGCUUCAAGGAAAGAGCCUCGG
CAUGAUCUUUGAGAAGCGCUCAACCAGGACCCGCCUUUCUACUGAAACUGGGUU
CGCGCUGCUCGGUGGCCACCCCUGCUUCCUGACGACCCAGGACAUCCACCUCGGA
GUGAACGAAUCCCUCACCGAUACCGCCCGGGUGUUAUCGAGCAUGGCAGAUGCCG
UGCUGGCCAGGGUGUACAAACAGUCCGAUCUGGACACUCUGGCCAAGGAGGCGU
CAAUUCCCAUCAUCAACGGCCUGAGCGACCUGUACCACCCAAUCCAAAUCCUGGC
UGACUACCUGACCCUGCAAGAGCACUACAGCAGCCUGAAGGGUCUGACCCUGUCA
UGGAUUGGCGAUGGAAACAAUAUUCUGCACUCCAUCAUGAUGUCCGCCGCGAAG
UUCGGAAUGCAUCUGCAAGCCGCCACGCCAAAAGGAUACGAACCGGAUGCGUCCG
UGACGAAGUUGGCGGAACAGUACGCGAAGGAGAACGGAACCAAGCUUCUGCUGA
CUAACGACCCCCUCGAGGCUGCGCAUGGGGGCAACGUGCUGAUUACCGACACCUG
GAUCUCCAUGGGGCAGGAGGAAGAGAAGAAGAAGAGACUGCAGGCAUUCCAGGG
GUACCAGGUCACCAUGAAAACCGCAAAAGUGGCAGCUUCGGACUGGACUUUCCU
GCAUUGCCUGCCGAGGAAGCCGGAGGAAGUCGACGACGAAGUGUUCUACUCGCC
UCGGUCCCUGGUGUUCCCCGAGGCCGAAAACCGGAAGUGGACCAUCAUGGCCGUG
AUGGUGUCCUUGCUGACUGACUAUAGCCCGCAGCUGCAGAAGCCUAAGUUCUAG
CUCGAGCUAGUGACUGACUAGGAUCUGGUUACCACUAAACCAGCCUCAAGAACA
CCCGAAUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUCUUCAC
AUUCUAG
>mARM709 (SEQ ID NO: 40)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCAUGCUUUUCAACCUGAGAAUCCUCUUGAACA
AUGCUGCUUUUCGGAAUGGCCACAACUUUAUGGUUCGGAACUUCCGUUGCGGCC
AGCCUUUACAAAACAAGGUCCAGCUGAAGGGCCGGGAUUUGCUCACACUGAAGA
ACUUUACUGGAGAAGAGAUCAAGUACAUGCUGUGGCUGUCGGCCGACCUGAAGU
UCAGGAUCAAGCAGAAGGGAGAAUACCUUCCGCUGCUUCAAGGAAAGAGCCUCG
GCAUGAUCUUUGAGAAGCGCUCAACCAGGACCCGCCUUUCUACUGAAACUGGGU
UCGCGCUGCUCGGUGGCCACCCCUGCUUCCUGACGACCCAGGACAUCCACCUCGG
AGUGAACGAAUCCCUCACCGAUACCGCCCGGGUGUUAUCGAGCAUGGCAGAUGCC
GUGCUGGCCAGGGUGUACAAACAGUCCGAUCUCGAUACCUUGGCAAAGGAGGCU
UCCAUUCCCAUCAUCAACGGCCUGAGCGACCUGUACCACCCAAUCCAAAUCCUGG
CUGACUACCUGACCCUGCAAGAGCACUACAGCAGCCUGAAGGGUCUGACCCUGUC
AUGGAUUGGCGAUGGAAACAAUAUUCUGCACUCCAUCAUGAUGUCCGCCGCGAA
GUUCGGAAUGCAUCUGCAAGCCGCCACUCCAAAAGGAUACGAACCGGAUGCGUCC
GUGACCAAGUUGGCGGAACAGUACGCGAAGGAGAACGGAACCAAGCUUCUGCUG
ACUAACGACCCCCUCGAGGCUGCGCAUGGGGGCAACGUGCUGAUUACCGACACCU
GGAUCUCCAUGGGGCAGGAGGAAGAGAAGAAGAAGAGACUGCAGGCAUUCCAGG
GGUACCAGGUCACCAUGAAAACCGCAAAAGUGGCAGCUUCGGACUGGACUUUCC
UGCAUUGCCUGCCGAGGAAGCCGGAGGAAGUCGACGACGAAGUGUUCUACUCGC
CUCGGUCCCUGGUGUUCCCCGAGGCCGAAAACCGGAAGUGGACCAUCAUGGCCGU
GAUGGUGUCCUUGCUGACUGACUAUAGCCCGCAGCUGCAGAAGCCUAAGUUCUA
GCUCGAGCUAGUGACUGACUAGGAUCUGGUUACCACUAAACCAGCCUCAAGAAC
ACCCGAAUGGAGUCUCUAAGCUACAUAAUACCAACUUACACUUACAAAAUGUUG
UCCCCCAAAAUGUAGCCAUUCGUAUCUGCUCCUAAUAAAAAGAAAGUUUCUUCA
CAUUCUAG
>mARM710 (SEQ ID NO: 41)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCAUGCUUUUCAACCUGAGAAUCCUCUUGAACA
AUGCUGCUUUUCGGAAUGGCCACAACUUUAUGGUUCGGAACUUCCGUUGCGGCC
AGCCUUUACAAAACAAGGUCCAGCUGAAGGGCCGGGAUUUGCUCACACUAAAGA
ACUUUACUGGAGAAGAGAUCAAGUACAUGCUAUGGCUGUCGGCCGACCUGAAGU
UCCGUAUCAAGCAGAAGGGAGAAUACCUUCCGCUGCUUCAAGGAAAGAGCCUCG
GCAUGAUCUUUGAGAAGCGCUCAACCAGGACCCGCCUUUCUACUGAAACUGGGU
UCGCGCUGCUCGGUGGCCACCCCUGCUUCCUGACGACCCAGGACAUCCACCUCGG
AGUGAACGAAUCCCUCACCGAUACCGCCCGGGUGUUAUCGAGCAUGGCAGAUGCC
GUGCUGGCCAGGGUGUACAAACAGUCCGAUCUCGAUACCUUGGCAAAGGAGGCU
UCCAUUCCCAUCAUCAACGGCCUGAGCGACCUGUACCACCCAAUCCAAAUCCUGG
CUGACUACCUGACCCUGCAAGAGCACUACAGCAGCCUGAAGGGUCUGACCCUGUC
AUGGAUUGGCGAUGGAAACAAUAUUCUGCACUCCAUCAUGAUGUCCGCCGCGAA
GUUCGGAAUGCAUCUGCAAGCCGCCACUCCAAAAGGAUACGAACCGGAUGCAUCC
GUGACCAAGUUGGCGGAACAGUACGCGAAGGAGAACGGAACCAAGCUCCUGCUG
ACUAACGACCCGCUCGAGGCUGCGCAUGGGGGUAACGUGCUGAUUACGGACACC
UGGAUCUCCAUGGGGCAGGAGGAAGAGAAGAAGAAGAGACUGCAGGCAUUCCAG
GGGUACCAGGUCACCAUGAAAACCGCAAAAGUGGCAGCUUCGGACUGGACUUUC
CUGCAUUGCCUGCCGAGGAAGCCGGAGGAAGUCGACGACGAAGUGUUCUACUCG
CCUCGGUCCCUGGUGUUCCCCGAGGCCGAAAACCGGAAGUGGACCAUCAUGGCCG
UGAUGGUGUCCUUGCUGACUGACUAUAGCCCGCAGCUGCAGAAGCCUAAGUUCU
AGCUCGAGCUAGUGACUGACUAGGAUCUGGUUACCACUAAACCAGCCUCAAGAA
CACCCGAAUGGAGUCUCUAAGCUACAUAAUACCAACUUACACUUACAAAAUGUU
GUCCCCCAAAAUGUAGCCAUUCGUAUCUGCUCCUAAUAAAAAGAAAGUUUCUUC
ACAUUCUAG
>mARM711 (SEQ ID NO: 42)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCAUGCUUUUCAACCUGAGAAUCCUCUUGAACA
AUGCUGCUUUUCGGAAUGGCCACAACUUUAUGGUUCGGAACUUCCGUUGCGGCC
AGCCUUUACAAAACAAGGUCCAGCUGAAGGGCCGGGAUUUGCUCACACUAAAGA
ACUUUACUGGAGAAGAGAUCAAGUACAUGCUAUGGCUGUCGGCCGACCUGAAGU
UCCGUAUCAAGCAGAAGGGAGAAUACCUUCCGCUGCUUCAAGGAAAGAGCCUCG
GCAUGAUCUUUGAGAAGCGCUCAACCAGGACCCGCCUUUCUACUGAAACUGGGU
UCGCGCUGCUCGGUGGCCACCCCUGCUUCCUGACGACCCAGGACAUCCACCUCGG
AGUGAACGAAUCCCUCACCGAUACCGCCCGGGUGUUAUCGAGCAUGGCAGAUGCC
GUGCUGGCCAGGGUGUACAAACAGUCCGAUCUCGAUACCUUGGCAAAGGAGGCU
UCCAUUCCCAUCAUCAACGGCCUGAGCGACCUGUACCACCCAAUCCAAAUCCUGG
CUGACUACCUGACCCUGCAAGAGCACUACAGCAGCCUGAAGGGUCUGACCCUGUC
AUGGAUUGGCGAUGGAAACAAUAUUCUGCACUCCAUCAUGAUGUCCGCCGCGAA
GUUCGGAAUGCAUCUGCAAGCCGCCACUCCAAAAGGAUACGAACCGGAUGCGUCC
GUGACCAAGUUGGCGGAACAGUACGCGAAGGAGAACGGAACCAAGCUUCUGCUG
ACUAACGACCCCCUCGAGGCUGCGCAUGGGGGCAACGUGCUGAUUACCGACACCU
GGAUCUCCAUGGGGCAGGAGGAAGAGAAGAAGAAGAGACUGCAGGCAUUCCAGG
GGUACCAGGUCACCAUGAAAACCGCAAAAGUGGCAGCUUCGGACUGGACUUUCC
UGCAUUGCCUGCCGAGGAAGCCGGAGGAAGUCGACGACGAAGUGUUCUACUCGC
CUCGGUCCCUGGUGUUCCCCGAGGCCGAAAACCGGAAGUGGACCAUCAUGGCCGU
GAUGGUGUCCUUGCUGACUGACUAUAGCCCGCAGCUGCAGAAGCCUAAGUUCUA
GCUCGAGCUAGUGACUGACUAGGAUCUGGUUACCACUAAACCAGCCUCAAGAAC
ACCCGAAUGGAGUCUCUAAGCUACAUAAUACCAACUUACACUUACAAAAUGUUG
UCCCCCAAAAUGUAGCCAUUCGUAUCUGCUCCUAAUAAAAAGAAAGUUUCUUCA
CAUUCUAG
>mARM712 (SEQ ID NO: 43)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUGUUCAACCUGCGAAUCCUGCUGAA
CAACGCCGCUUUUCGGAACGGGCACAACUUUAUGGUGAGGAACUUUCGCUGCGG
ACAGCCCCUCCAGAAUAAGGUCCAGCUGAAGGGCAGGGACCUGCUGACCCUGAAA
AAUUUCACAGGGGAGGAAAUCAAGUAUAUGCUGUGGCUGUCAGCUGAUCUGAAG
UUCCGGAUCAAGCAGAAGGGCGAAUAUCUGCCUCUGCUCCAGGGCAAAAGCCUG
GGGAUGAUCUUCGAAAAGCGCAGUACUCGGACCAGACUGUCAACCGAGACUGGA
UUCGCUCUGCUGGGAGGACACCCUUGUUUUCUGACCACUCAGGACAUUCACCUGG
GAGUGAACGAGUCCCUGACCGACACUGCUCGCGUCCUGAGCUCUAUGGCCGACGC
UGUGCUGGCUCGAGUCUACAAACAGUCCGACCUGGAUACCCUGGCCAAGGAAGC
UUCUAUCCCAAUUAUUAACGGCCUGUCAGACCUGUAUCACCCCAUCCAGAUUCUG
GCCGAUUACCUGACCCUCCAGGAGCACUAUUCUAGUCUGAAAGGGCUGACACUG
AGUUGGAUUGGGGACGGAAACAAUAUCCUGCACUCUAUUAUGAUGUCAGCCGCC
AAGUUUGGAAUGCACCUCCAGGCUGCAACCCCAAAAGGCUACGAACCCGAUGCCU
CAGUGACAAAGCUGGCUGAACAGUACGCCAAAGAGAACGGCACUAAGCUGCUGC
UGACCAACGACCCUCUGGAGGCCGCUCACGGAGGCAACGUGCUGAUCACCGAUAC
CUGGAUUAGUAUGGGACAGGAGGAAGAGAAGAAGAAGCGGCUCCAGGCCUUCCA
GGGCUACCAGGUGACAAUGAAAACCGCUAAGGUCGCAGCCAGCGAUUGGACCUU
UCUGCACUGCCUGCCCAGAAAGCCCGAAGAGGUGGACGACGAGGUCUUCUACUCU
CCCAGAAGCCUGGUGUUUCCCGAAGCUGAGAAUAGGAAGUGGACAAUUAUGGCA
GUGAUGGUCAGCCUGCUGACUGAUUAUUCACCUCAGCUCCAGAAACCAAAGUUC
UGAUAACUCGAGCUAGUGACUGACUAGGAUCUGGUUACCACUAAACCAGCCUCA
AGAACACCCGAAUGGAGUCUCUAAGCUACAUAAUACCAACUUACACUUACAAAA
UGUUGUCCCCCAAAAUGUAGCCAUUCGUAUCUGCUCCUAAUAAAAAGAAAGUUU
CUUCACAUUCUAG
>mARM713 (SEQ ID NO: 44)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUGUUCAACCUGCGCAUCCUGCUGAA
CAACGCCGCCUUCCGCAACGGCCACAACUUCAUGGUGCGCAACUUCCGCUGCGGC
CAGCCCCUGCAGAACAAGGUGCAGCUGAAGGGCCGCGACCUGCUGACCCUGAAGA
ACUUCACCGGCGAGGAGAUCAAGUACAUGCUGUGGCUGAGCGCCGACCUGAAGU
UCCGCAUCAAGCAGAAGGGCGAGUACCUGCCCCUGCUGCAGGGCAAGAGCCUGGG
CAUGAUCUUCGAGAAGCGCAGCACCCGCACCCGCCUGAGCACCGAGACAGGCCUG
GCCCUGCUGGGCGGCCACCCCUGCUUCCUGACCACCCAGGACAUCCACCUGGGCG
UGAACGAGAGCCUGACCGACACCGCCCGCGUGCUGAGCAGCAUGGCCGACGCCGU
GCUGGCCCGCGUGUACAAGCAGAGCGACCUGGACACCCUGGCCAAGGAGGCCAGC
AUCCCCAUCAUCAACGGCCUGAGCGACCUGUACCACCCCAUCCAGAUCCUGGCCG
ACUACCUGACCCUGCAGGAGCACUACAGCAGCCUGAAGGGCCUGACCCUGAGCUG
GAUCGGCGACGGCAACAACAUCCUGCACAGCAUCAUGAUGAGCGCCGCCAAGUUC
GGCAUGCACCUGCAGGCCGCCACCCCCAAGGGCUACGAGCCCGACGCCAGCGUGA
CCAAGCUGGCCGAGCAGUACGCCAAGGAGAACGGCACCAAGCUGCUGCUGACCAA
CGACCCCCUGGAGGCCGCCCACGGCGGCAACGUGCUGAUCACCGACACCUGGAUC
AGCAUGGGCCAGGAGGAGGAGAAGAAGAAGCGCCUGCAGGCCUUCCAGGGCUAC
CAGGUGACCAUGAAGACCGCCAAGGUGGCCGCCAGCGACUGGACCUUCCUGCACU
GCCUGCCCCGCAAGCCCGAGGAGGUGGACGACGAGGUGUUCUACAGCCCCCGCAG
CCUGGUGUUCCCCGAGGCCGAGAACCGCAAGUGGACCAUCAUGGCCGUGAUGGU
GAGCCUGCUGACCGACUACAGCCCCCAGCUGCAGAAGCCCAAGUUCUGAUAACUC
GAGCUAGUGACUGACUAGGAUCUGGUUACCACUAAACCAGCCUCAAGAACACCC
GAAUGGAGUCUCUAAGCUACAUAAUACCAACUUACACUUACAAAAUGUUGUCCC
CCAAAAUGUAGCCAUUCGUAUCUGCUCCUAAUAAAAAGAAAGUUUCUUCACAUU
CUAG
>mARM714 (SEQ ID NO: 45)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUGUUCAACCUGCGAAUCCUGCUGAA
CAACGCCGCUUUUCGGAACGGGCACAACUUUAUGGUGAGGAACUUUCGCUGCGG
ACAGCCCCUCCAGAAUAAGGUCCAGCUGAAGGGCAGGGACCUGCUGACCCUGAAA
AAUUUCACAGGGGAGGAAAUCAAGUAUAUGCUGUGGCUGUCAGCUGAUCUGAAG
UUCCGGAUCAAGCAGAAGGGCGAAUAUCUGCCUCUGCUCCAGGGCAAAAGCCUG
GGGAUGAUCUUCGAAAAGCGCAGUACUCGGACCAGACUGUCAACCGAGACUGGA
UUCGCUCUGCUGGGAGGACACCCUUGUUUUCUGACCACUCAGGACAUUCACCUGG
GAGUGAACGAGUCCCUGACCGACACUGCUCGCGUCCUGAGCUCUAUGGCCGACGC
UGUGCUAGCUCGAGUCUACAAACAGUCCGACCUGGAUACCCUGGCCAAGGAAGC
UUCUAUCCCAAUUAUUAACGGCCUGUCAGACCUGUAUCACCCCAUCCAGAUUCUG
GCCGAUUACCUGACCCUCCAGGAGCACUAUUCUAGUCUGAAAGGGCUGACACUG
AGUUGGAUUGGGGACGGAAACAAUAUCCUGCACUCUAUUAUGAUGUCAGCCGCC
AAGUUUGGAAUGCACCUCCAGGCUGCAACCCCAAAAGGCUACGAACCCGAUGCCU
CAGUGACAAAGCUGGCUGAACAGUACGCCAAAGAGAACGGCACUAAGCUGCUGC
UGACCAACGACCCUCUGGAGGCCGCUCACGGAGGCAACGUGCUGAUCACCGAUAC
CUGGAUUAGUAUGGGACAGGAGGAAGAGAAGAAGAAGCGGCUCCAGGCCUUCCA
GGGCUACCAGGUGACAAUGAAAACCGCUAAGGUCGCAGCCAGCGAUUGGACCUU
UCUGCACUGCCUGCCCAGAAAGCCCGAAGAGGUGGACGACGAGGUCUUCUACUCU
CCCAGAAGCCUGGUGUUUCCCGAAGCUGAGAAUAGGAAGUGGACAAUUAUGGCA
GUGAUGGUCAGCCUGCUGACUGAUUAUUCACCUCAGCUCCAGAAACCAAAGUUC
UGAUAACUCGAGCUAGUGACUGACUAGGAUCUGGUUACCACUAAACCAGCCUCA
AGAACACCCGAAUGGAGUCUCUAAGCUACAUAAUACCAACUUACACUUACAAAA
UGUUGUCCCCCAAAAUGUAGCCAUUCGUAUCUGCUCCUAAUAAAAAGAAAGUUU
CUUCACAUUCUAG
>mARM715 (SEQ ID NO: 46)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUGUUCAACCUGCGAAUCCUGCUGAA
CAAUGCCGCUUUUCGGAACGGGCACAAUUUCAUGGUGAGGAACUUUCGCUGCGG
ACAGCCCCUCCAGAACAAGGUCCAGCUGAAGGGCAGGGACCUGCUGACCCUGAAA
AAUUUCACAGGGGAGGAAAUCAAGUACAUGCUGUGGCUGUCAGCCGAUCUGAAG
UUCCGGAUCAAGCAGAAGGGCGAAUAUCUGCCUCUGCUCCAGGGCAAAAGCCUG
GGGAUGAUCUUCGAAAAGCGCAGUACUCGGACCAGACUGUCAACAGAGACUGGA
UUCGCACUGCUGGGAGGACACCCAUGUUUUCUGACCACACAGGACAUUCAUCUG
GGAGUGAACGAGUCCCUGACCGACACAGCACGCGUCCUGAGCUCCAUGGCUGAUG
CAGUGCUGGCUCGAGUCUACAAACAGUCUGACCUGGAUACCCUGGCCAAGGAAG
CUUCUAUCCCAAUCAUUAAUGGCCUGAGUGACCUGUAUCACCCCAUCCAGAUUCU
GGCCGAUUACCUGACCCUCCAGGAGCAUUAUUCUAGUCUGAAAGGGCUGACACU
GAGCUGGAUUGGGGACGGAAACAAUAUCCUGCACUCCAUUAUGAUGAGCGCCGC
CAAGUUUGGAAUGCACCUCCAGGCUGCAACCCCAAAAGGCUACGAACCCGAUGCC
UCCGUGACAAAGCUGGCAGAACAGUAUGCCAAAGAGAACGGCACUAAGCUGCUG
CUGACCAAUGACCCUCUGGAGGCCGCUCACGGAGGCAACGUGCUGAUCACUGAUA
CCUGGAUUAGUAUGGGACAGGAGGAAGAGAAGAAGAAGCGGCUCCAGGCCUUCC
AGGGCUACCAGGUGACAAUGAAAACUGCUAAGGUCGCAGCCAGCGACUGGACCU
UUCUGCAUUGCCUGCCCAGAAAGCCUGAAGAGGUGGACGAUGAGGUCUUCUACU
CACCCAGAAGCCUGGUGUUUCCUGAAGCUGAGAAUAGGAAGUGGACAAUCAUGG
CAGUGAUGGUCAGCCUGCUGACUGAUUAUUCCCCUCAGCUCCAGAAACCAAAGU
UCUGAUAACUCGAGCUAGUGACUGACUAGGAUCUGGUUACCACUAAACCAGCCU
CAAGAACACCCGAAUGGAGUCUCUAAGCUACAUAAUACCAACUUACACUUACAA
AAUGUUGUCCCCCAAAAUGUAGCCAUUCGUAUCUGCUCCUAAUAAAAAGAAAGU
UUCUUCACAUUCUAG
>mARM716 (SEQ ID NO: 47)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAACCUUCGCAUUCUCCUCAA
CAACGCCGCGUUUAGAAACGGACACAACUUCAUGGUCCGCAACUUCCGCUGCGGA
CAGCCGCUGCAGAACAAGGUCCAGCUCAAGGGUCGGGAUCUCCUGACGCUGAAG
AACUUUACCGGCGAAGAGAUUAAGUACAUGCUGUGGCUGUCCGCCGACCUUAAG
UUCCGGAUCAAGCAGAAGGGCGAAUACCUUCCCCUGCUGCAAGGAAAGUCCCUG
GGCAUGAUCUUCGAGAAGCGCAGUACCAGAACCAGACUCUCCACUGAAACCGGG
UUCGCGCUGCUUGGCGGCCACCCGUGUUUCCUCACUACGCAAGACAUCCAUCUUG
GCGUGAACGAGUCCCUUACCGACACCGCCAGGGUGCUGUCAAGCAUGGCCGACGC
CGUCCUUGCGCGCGUGUACAAGCAGUCAGACCUUGAUACUCUGGCCAAGGAAGCC
UCCAUCCCUAUUAUCAACGGCCUAUCCGACCUUUACCACCCGAUCCAGAUCCUCG
CUGACUACCUGACCCUGCAAGAACACUACAGCAGCCUCAAGGGACUGACUCUGUC
CUGGAUCGGCGACGGGAACAACAUCCUGCACUCAAUCAUGAUGAGCGCAGCCAA
GUUCGGCAUGCAUCUCCAAGCCGCUACACCCAAGGGUUAUGAACCGGACGCCUCU
GUGACCAAGUUGGCAGAACAGUACGCCAAGGAGAACGGUACUAAGCUCCUUUUA
ACCAACGACCCCCUCGAAGCAGCCCAUGGCGGGAAUGUGCUCAUUACCGAUACCU
GGAUUUCGAUGGGCCAGGAGGAGGAGAAGAAGAAGCGGCUGCAGGCGUUCCAGG
GCUACCAGGUCACCAUGAAAACUGCCAAAGUGGCCGCCUCGGAUUGGACCUUUCU
CCACUGCCUGCCUCGGAAGCCUGAGGAGGUGGACGACGAAGUGUUCUACUCCCCA
CGGUCCCUCGUGUUCCCCGAGGCCGAAAAUAGGAAGUGGACCAUCAUGGCCGUG
AUGGUGUCCCUCUUGACCGAUUACAGCCCGCAGCUUCAGAAGCCUAAAUUCUAGC
UCGAGCUAGUGACUGACUAGGAUCUGGUUACCACUAAACCAGCCUCAAGAACAC
CCGAAUGGAGUCUCUAAGCUACAUAAUACCAACUUACACUUACAAAAUGUUGUC
CCCCAAAAUGUAGCCAUUCGUAUCUGCUCCUAAUAAAAAGAAAGUUUCUUCACA
UUCUAG
>mARM717 (SEQ ID NO: 48)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAAUCUUCGCAUCCUGUUGA
ACAACGCCGCCUUCCGCAAUGGUCACAACUUCAUGGUCCGGAACUUCAGAUGUGG
ACAGCCUCUCCAAAACAAGGUCCAGCUGAAGGGAAGGGACCUCUUAACCCUCAAA
AACUUUACUGGAGAGGAGAUCAAGUACAUGCUGUGGCUUAGCGCCGACCUUAAG
UUCCGGAUCAAGCAGAAGGGAGAGUACCUCCCGCUGCUGCAAGGAAAGAGUCUU
GGAAUGAUCUUCGAGAAGCGGUCCACCAGAACUCGCCUCUCCACUGAAACCGGAU
UCGCACUCCUGGGUGGACACCCGUGCUUUCUGACCACCCAAGACAUCCACCUCGG
AGUGAACGAGAGCCUCACGGACACCGCGAGAGUGCUGUCAUCCAUGGCCGACGCC
GUGCUUGCACGGGUCUACAAGCAGUCCGAUCUGGACACUCUUGCCAAGGAAGCC
UCCAUUCCUAUCAUUAACGGUCUGUCGGAUCUGUACCACCCGAUUCAGAUCCUUG
CGGACUACCUCACACUUCAAGAACACUAUUCAAGCCUAAAGGGUCUGACCCUGUC
CUGGAUCGGAGAUGGAAACAACAUUCUCCAUUCCAUCAUGAUGAGCGCUGCCAA
GUUCGGAAUGCAUCUCCAAGCAGCGACUCCUAAGGGUUACGAGCCGGACGCCUCA
GUGACUAAGCUGGCCGAGCAGUACGCCAAGGAGAACGGUACCAAACUGUUGCUU
ACUAACGACCCGCUUGAAGCGGCCCAUGGAGGAAACGUGCUGAUUACCGACACCU
GGAUUUCGAUGGGACAGGAAGAGGAGAAGAAGAAGCGGCUCCAGGCGUUCCAGG
GAUACCAGGUCACCAUGAAAACGGCCAAAGUGGCCGCUAGCGAUUGGACCUUUC
UGCACUGCCUCCCGCGCAAGCCUGAAGAAGUGGACGACGAAGUGUUCUACUCCCC
UCGCUCUCUUGUGUUCCCGGAAGCCGAAAACAGGAAGUGGACCAUCAUGGCCGU
GAUGGUGUCCCUCCUGACCGAUUACAGCCCGCAGCUGCAGAAGCCUAAGUUCUAG
CUCGAGCUAGUGACUGACUAGGAUCUGGUUACCACUAAACCAGCCUCAAGAACA
CCCGAAUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUCUUCAC
AUUCUAG
>mARM718 (SEQ ID NO: 49)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAAUCUCCGCAUCCUCCUCAA
CAACGCCGCGUUUAGAAACGGCCACAACUUCAUGGUCCGGAACUUCAGAUGUGG
CCAGCCGCUUCAGAACAAGGUCCAGCUCAAGGGCCGGGAUCUUCUGACCCUGAAG
AACUUUACUGGCGAAGAAAUCAAGUACAUGCUCUGGCUCUCCGCCGACUUGAAG
UUCCGCAUUAAGCAGAAGGGGGAAUACCUUCCGCUGCUGCAAGGAAAGUCGCUC
GGCAUGAUCUUUGAGAAGCGCUCAACCCGCACCAGGCUGUCCACUGAAACCGGGU
UCGCGCUGCUUGGUGGCCACCCCUGCUUCCUGACCACCCAAGACAUUCACCUCGG
AGUGAACGAAUCGCUCACUGAUACUGCCCGGGUGCUGUCGUCGAUGGCCGAUGC
AGUGCUGGCCAGGGUGUACAAACAGUCCGAUCUGGACACUCUGGCCAAGGAGGC
GUCCAUCCCUAUUAUCAACGGCCUUUCCGACCUCUACCACCCGAUUCAGAUCCUU
GCCGAUUACCUCACCCUGCAAGAACACUACUCGUCACUGAAGGGUCUGACCUUGU
CCUGGAUCGGCGACGGCAACAACAUCCUCCAUUCCAUUAUGAUGUCCGCCGCCAA
AUUCGGCAUGCAUCUUCAAGCCGCAACCCCUAAGGGUUACGAGCCGGACGCUUCC
GUGACCAAGCUCGCCGAGCAGUACGCUAAGGAGAACGGAACCAAGCUUCUGCUG
ACUAACGACCCCCUAGAGGCAGCCCACGGGGGCAACGUGCUUAUUACUGACACCU
GGAUCUCCAUGGGACAGGAAGAAGAGAAGAAGAAGCGGUUACAGGCGUUCCAGG
GCUAUCAGGUCACCAUGAAAACCGCCAAGGUCGCUGCCUCGGACUGGACCUUCCU
GCAUUGCCUGCCUCGCAAGCCCGAAGAAGUGGACGACGAGGUGUUCUACUCGCCA
CGGUCCCUUGUGUUCCCUGAGGCCGAGAAUAGAAAGUGGACCAUUAUGGCCGUG
AUGGUGUCCCUUCUCACCGACUACUCGCCGCAACUGCAGAAACCCAAGUUCUAGC
UCGAGCUAGUGACUGACUAGGAUCUGGUUACCACUAAACCAGCCUCAAGAACAC
CCGAAUGGAGUCUCUAAGCUACAUAAUACCAACUUACACUUACAAAAUGUUGUC
CCCCAAAAUGUAGCCAUUCGUAUCUGCUCCUAAUAAAAAGAAAGUUUCUUCACA
UUCUAG
>mARM719 (SEQ ID NO: 50)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAAUCUUCGCAUCCUCCUCAA
CAACGCCGCCUUCCGGAACGGUCACAACUUCAUGGUCCGGAACUUCCGCUGCGGC
CAGCCGCUCCAAAACAAAGUGCAGCUUAAGGGCCGCGAUCUCCUGACCCUGAAGA
ACUUCACCGGAGAGGAAAUCAAGUACAUGCUGUGGCUCUCGGCGGACCUGAAGU
UUAGGAUUAAGCAGAAGGGGGAGUAUCUGCCGCUGCUCCAAGGGAAGUCCCUUG
GCAUGAUCUUCGAAAAGAGGUCCACCCGGACUCGGCUCAGCACCGAAACAGGUU
UUGCACUUCUGGGGGGCCACCCGUGCUUCCUGACGACCCAGGACAUCCAUCUGGG
UGUCAACGAGAGUUUGACCGACACUGCCAGAGUGCUGUCAUCCAUGGCGGACGC
GGUGCUCGCGAGAGUGUACAAGCAGUCCGAUCUUGACACCCUGGCAAAAGAGGC
UUCAAUCCCGAUCAUUAACGGACUCUCGGAUCUGUACCACCCUAUCCAAAUCUUG
GCCGACUACCUGACCCUGCAAGAACACUACAGCUCCCUGAAGGGCCUGACUCUUU
CCUGGAUUGGCGAUGGAAACAACAUUCUCCAUUCUAUUAUGAUGUCCGCCGCCA
AGUUCGGCAUGCACCUUCAAGCCGCCACCCCGAAGGGCUACGAACCUGACGCCUC
CGUGACUAAGCUAGCCGAACAGUACGCUAAGGAGAACGGCACUAAGCUUCUCCU
UACCAACGAUCCGCUGGAGGCGGCCCAUGGCGGAAAUGUGCUUAUCACCGACACC
UGGAUUAGCAUGGGGCAGGAAGAAGAGAAGAAGAAACGGCUCCAGGCAUUCCAG
GGCUACCAGGUCACCAUGAAAACUGCCAAGGUCGCCGCUAGCGACUGGACCUUCC
UCCACUGUCUGCCUCGCAAGCCUGAAGAAGUGGACGACGAGGUGUUCUACUCCCC
GCGCUCCCUCGUGUUUCCUGAGGCCGAGAACAGAAAGUGGACCAUCAUGGCCGU
GAUGGUGUCAUUACUUACGGACUACAGCCCGCAGCUGCAGAAGCCGAAGUUCUA
GCUCGAGCUAGUGACUGACUAGGAUCUGGUUACCACUAAACCAGCCUCAAGAAC
ACCCGAAUGGAGUCUCUAAGCUACAUAAUACCAACUUACACUUACAAAAUGUUG
UCCCCCAAAAUGUAGCCAUUCGUAUCUGCUCCUAAUAAAAAGAAAGUUUCUUCA
CAUUCUAG
>mARM720 (SEQ ID NO: 51)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUUAACUUGAGAAUCCUUCUGA
ACAACGCCGCUUUCCGCAACGGUCAUAACUUCAUGGUCCGGAACUUCAGAUGUG
GCCAGCCCCUCCAAAACAAAGUGCAGCUGAAGGGCCGGGACCUUCUUACGCUGAA
GAAUUUCACCGGCGAAGAAAUCAAGUACAUGCUCUGGCUGUCCGCCGAUCUUAA
GUUCCGCAUUAAGCAGAAGGGGGAAUACCUCCCGCUGCUGCAAGGGAAGUCGCU
GGGCAUGAUUUUUGAGAAGCGGUCAACUCGCACCCGCCUGUCCACUGAAACUGG
AUUCGCACUGCUCGGUGGCCAUCCCUGCUUCCUGACCACCCAAGACAUCCACCUC
GGCGUGAACGAGUCCCUGACUGACACCGCCCGGGUCUUAUCCUCGAUGGCCGAUG
CUGUGCUUGCGAGGGUGUACAAGCAGUCCGACCUCGACACACUCGCGAAGGAGG
CCUCCAUCCCCAUCAUCAACGGCCUGUCCGACCUUUACCACCCAAUUCAGAUCCU
CGCCGAUUACCUGACCCUGCAAGAGCACUACUCGUCGCUCAAGGGGCUUACCCUC
UCGUGGAUUGGCGACGGCAACAACAUCCUUCACUCCAUCAUGAUGUCGGCAGCG
AAGUUCGGCAUGCAUCUGCAAGCCGCCACGCCUAAGGGUUAUGAACCGGAUGCC
UCAGUGACCAAGCUCGCCGAACAGUACGCGAAAGAGAAUGGAACCAAGCUACUU
CUGACCAACGACCCCCUGGAGGCCGCUCACGGCGGCAACGUCCUCAUUACCGAUA
CUUGGAUUUCGAUGGGACAGGAAGAGGAAAAGAAGAAGAGACUGCAGGCGUUCC
AGGGAUACCAGGUCACCAUGAAAACUGCCAAAGUGGCAGCCUCCGACUGGACCU
UCCUUCACUGCCUGCCGAGGAAGCCUGAAGAGGUGGACGACGAGGUGUUCUACU
CCCCGCGCUCCUUGGUGUUUCCUGAGGCCGAAAACCGGAAGUGGACUAUCAUGGC
CGUGAUGGUGUCCCUCCUCACCGACUACUCGCCGCAACUGCAGAAGCCUAAGUUC
UAGCUCGAGCUAGUGACUGACUAGGAUCUGGUUACCACUAAACCAGCCUCAAGA
ACACCCGAAUGGAGUCUCUAAGCUACAUAAUACCAACUUACACUUACAAAAUGU
UGUCCCCCAAAAUGUAGCCAUUCGUAUCUGCUCCUAAUAAAAAGAAAGUUUCUU
CACAUUCUAG
>mARM721 (SEQ ID NO: 52)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGUUAUUCAACCUUAGAAUUCUCCUUA
ACAACGCCGCCUUCCGGAAUGGGCAUAACUUUAUGGUCCGCAAUUUCCGCUGUG
GACAGCCUCUGCAAAACAAGGUCCAGCUCAAGGGCCGGGAUCUGCUGACUCUCAA
GAACUUCACUGGGGAAGAAAUCAAGUACAUGCUCUGGCUGAGCGCCGACCUCAA
GUUCCGCAUCAAGCAGAAGGGAGAGUACCUCCCGCUGCUCCAAGGGAAGUCCCUG
GGCAUGAUCUUCGAGAAGAGAUCCACCCGCACCAGACUUUCCACUGAGACUGGCU
UCGCCUUGCUGGGAGGCCACCCAUGCUUCCUGACGACCCAGGACAUUCACCUUGG
CGUGAACGAGUCCCUGACUGACACCGCAAGGGUGUUGUCCUCGAUGGCCGACGCC
GUGCUUGCCCGGGUGUACAAGCAGAGCGAUCUUGACACCCUGGCUAAGGAAGCU
UCCAUUCCCAUCAUCAACGGUCUGAGCGACCUGUACCACCCGAUUCAGAUCCUGG
CGGACUACCUAACCCUGCAAGAGCACUAUAGCUCCCUGAAGGGCCUCACACUUUC
AUGGAUCGGCGACGGCAACAACAUCCUGCACUCUAUUAUGAUGAGCGCUGCCAA
AUUCGGCAUGCACCUCCAAGCCGCCACGCCUAAAGGCUACGAGCCCGACGCCUCG
GUGACCAAGCUUGCGGAGCAGUACGCGAAGGAAAACGGCACCAAGCUGCUUCUC
ACCAACGAUCCUCUGGAAGCGGCCCAUGGUGGCAACGUGCUCAUUACCGACACUU
GGAUCUCCAUGGGACAGGAGGAGGAAAAGAAGAAGCGGCUCCAGGCGUUUCAGG
GUUACCAGGUCACCAUGAAAACCGCCAAGGUCGCAGCCUCCGACUGGACCUUCCU
UCAUUGCCUUCCGCGCAAGCCCGAAGAAGUGGACGAUGAAGUGUUUUACUCACC
UCGGUCACUCGUGUUCCCGGAAGCAGAGAACAGGAAAUGGACCAUUAUGGCCGU
GAUGGUGUCCCUGCUCACCGAUUACAGUCCGCAACUGCAGAAGCCCAAGUUCUAG
CUCGAGCUAGUGACUGACUAGGAUCUGGUUACCACUAAACCAGCCUCAAGAACA
CCCGAAUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUCUUCAC
AUUCUAG
>mARM722 (SEQ ID NO: 53)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAAUCUCCGCAUCCUCCUUAA
CAACGCCGCGUUUAGAAACGGCCACAACUUCAUGGUCCGGAACUUCAGAUGUGG
CCAGCCGCUUCAAAACAAGGUCCAGCUGAAGGGCCGGGAUCUUCUGACCCUGAAG
AACUUUACUGGCGAAGAGAUCAAGUACAUGCUCUGGCUCUCCGCGGACUUGAAG
UUCCGCAUUAAGCAGAAGGGGGAAUACCUUCCGCUGCUUCAAGGAAAGAGCCUC
GGCAUGAUCUUUGAGAAGCGCUCAACCAGGACCCGCCUUUCUACUGAAACUGGG
UUCGCGCUGCUCGGUGGCCACCCCUGCUUCCUGACGACCCAGGACAUCCACCUCG
GAGUGAACGAAUCCCUCACCGAUACCGCCCGGGUGUUAUCGAGCAUGGCAGAUG
CCGUGCUGGCCAGGGUGUACAAACAGUCCGAUCUGGACACUCUGGCCAAGGAGG
CGUCAAUUCCUAUUAUCAACGGCCUUAGUGACCUCUACCAUCCGAUUCAAAUCCU
GGCCGAUUACCUCACCCUGCAAGAACACUACAGCUCCCUGAAGGGUCUGACAUUG
UCCUGGAUCGGCGACGGCAACAACAUUCUCCAUUCCAUCAUGAUGUCCGCCGCAA
AAUUCGGCAUGCAUCUUCAAGCCGCCACGCCUAAGGGUUACGAACCCGACGCUUC
CGUGACUAAGCUCGCCGAGCAGUACGCUAAGGAGAACGGAACCAAGCUGCUGCU
GACUAACGACCCGCUAGAAGCAGCCCACGGGGGCAACGUGCUUAUUACUGACACC
UGGAUCUCCAUGGGCCAGGAGGAAGAGAAAAAGAAGCGGCUGCAGGCGUUCCAG
GGAUAUCAGGUCACCAUGAAAACCGCCAAGGUCGCUGCCUCCGACUGGACCUUCC
UGCACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCCGU
GAUGGUGUCACUUCUCACCGACUACAGCCCGCAGCUUCAGAAGCCCAAGUUCUAG
CUCGAGCUAGUGACUGACUAGGAUCUGGUUACCACUAAACCAGCCUCAAGAACA
CCCGAAUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUCUUCAC
AUUCUAG
>mARM723 (SEQ ID NO: 54)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAAUCUCCGCAUCCUCCUUAA
CAACGCCGCGUUUAGAAACGGCCACAACUUCAUGGUCCGGAACUUCAGAUGUGG
CCAGCCGCUUCAAAACAAGGUCCAGCUUAAGGGCCGGGAUCUCCUCACCCUUAAA
AACUUCACCGGCGAAGAGAUCAAGUACAUGCUCUGGCUCUCCGCGGACCUUAAG
UUCCGCAUUAAGCAGAAGGGGGAAUACCUUCCGCUGCUUCAAGGAAAGAGCCUC
GGCAUGAUCUUUGAGAAGCGCUCAACCAGGACCAGGCUUUCUACUGAAACUGGG
UUCGCGCUUCUCGGCGGUCAUCCCUGCUUCCUCACGACCCAAGACAUCCACCUCG
GAGUGAACGAAUCCCUCACGGAUACUGCCCGCGUGCUUUCGAGCAUGGCAGACGC
CGUGCUCGCCCGGGUGUACAAACAGUCCGAUCUCGACACUCUCGCCAAGGAGGCG
UCAAUUCCUAUUAUCAACGGUCUUAGUGACCUUUACCACCCGAUCCAGAUCCUCG
CCGAUUACCUCACACUCCAAGAACACUACAGCUCCCUUAAGGGUCUUACCCUCUC
CUGGAUCGGCGACGGCAACAACAUUCUCCACUCCAUCAUGAUGUCCGCCGCAAAG
UUCGGCAUGCAUCUUCAAGCCGCCACCCCGAAGGGCUACGAGCCUGAUGCUUCCG
UGACUAAGCUCGCCGAGCAGUACGCUAAGGAGAACGGAACCAAGCUUCUUCUCA
CUAACGACCCACUCGAAGCAGCCCAUGGGGGCAACGUGCUUAUCACUGACACCUG
GAUCUCCAUGGGCCAGGAAGAAGAGAAGAAGAAGCGGCUCCAGGCGUUCCAGGG
AUAUCAGGUCACCAUGAAAACCGCCAAGGUCGCUGCCUCCGACUGGACCUUUCUC
CACUGCCUCCCUCGCAAACCUGAAGAAGUGGACGACGAGGUGUUCUACUCGCCCC
GGAGCCUCGUGUUCCCCGAGGCCGAGAAUAGAAAGUGGACCAUUAUGGCCGUGA
UGGUGUCACUCCUCACCGACUACAGCCCGCAGCUUCAGAAGCCCAAGUUCUAGCU
CGAGCUAGUGACUGACUAGGAUCUGGUUACCACUAAACCAGCCUCAAGAACACCC
GAAUGGAGUCUCUAAGCUACAUAAUACCAACUUACACUUACAAAAUGUUGUCCC
CCAAAAUGUAGCCAUUCGUAUCUGCUCCUAAUAAAAAGAAAGUUUCUUCACAUU
CUAG
>mARM724 (SEQ ID NO: 55)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAAUCUCCGCAUCCUCCUUAA
CAACGCCGCGUUUAGAAACGGACAUAACUUCAUGGUCCGGAACUUCAGAUGUGG
ACAGCCGCUUCAAAACAAGGUCCAGCUGAAGGGUCGGGAUCUUCUGACCCUGAA
GAACUUUACCGGAGAAGAGAUCAAGUACAUGCUCUGGCUCUCCGCGGACUUGAA
GUUCCGCAUUAAGCAGAAGGGAGAAUACCUCCCGCUGCUUCAAGGAAAGAGCCU
CGGAAUGAUUUUUGAGAAGCGCUCAACCAGGACCCGCCUUUCUACUGAAACUGG
AUUCGCGCUGCUGGGUGGACACCCCUGCUUCCUGACGACCCAGGACAUCCACCUC
GGAGUGAACGAAUCCCUCACUGAUACCGCCCGGGUGUUAUCGAGCAUGGCAGAU
GCCGUGCUGGCCAGGGUGUACAAACAGUCCGAUCUGGACACUCUGGCCAAGGAG
GCGUCAAUUCCUAUCAUCAACGGACUUAGUGACCUCUACCAUCCGAUUCAAAUCC
UGGCCGACUACCUCACCCUGCAAGAACACUACAGCUCCCUGAAGGGUCUGACAUU
GUCCUGGAUCGGAGAUGGAAACAACAUUCUCCACUCCAUCAUGAUGUCCGCCGCA
AAAUUCGGAAUGCAUCUUCAAGCCGCCACGCCUAAGGGUUACGAACCCGACGCUU
CCGUGACUAAGCUCGCCGAGCAGUACGCUAAGGAGAACGGUACCAAGCUUCUCCU
GACCAACGACCCACUAGAAGCAGCCCACGGUGGAAACGUGCUUAUUACUGACACU
UGGAUCUCCAUGGGACAGGAGGAAGAGAAAAAGAAGCGGCUGCAGGCGUUCCAG
GGAUAUCAGGUCACCAUGAAAACCGCCAAGGUCGCUGCCUCCGACUGGACCUUCC
UGCACUGCCUGCCUCGCAAGCCUGAAGAAGUGGACGACGAGGUGUUCUACUCGCC
GCGGAGCCUCGUGUUCCCCGAGGCCGAGAAUAGAAAGUGGACCAUCAUGGCCGU
GAUGGUGUCACUGCUCACCGACUACAGCCCGCAGCUUCAGAAGCCCAAGUUCUAG
CUCGAGCUAGUGACUGACUAGGAUCUGGUUACCACUAAACCAGCCUCAAGAACA
CCCGAAUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUCUUCAC
AUUCUAG
>mARM725 (SEQ ID NO: 56)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAACCUCCGCAUUCUCCUCAA
CAACGCUGCCUUCCGGAAUGGACAUAACUUCAUGGUCCGGAACUUCAGAUGCGG
ACAGCCGCUUCAGAACAAGGUCCAGCUUAAGGGGAGAGAUCUCCUUACCCUCAA
AAACUUCACUGGCGAAGAAAUCAAGUACAUGCUCUGGCUUAGUGCGGAUCUCAA
GUUCCGCAUCAAGCAGAAGGGAGAAUACCUCCCGCUCCUUCAAGGAAAGAGCCUC
GGCAUGAUUUUUGAGAAGAGGUCCACCAGAACUCGCCUUUCAACCGAGACUGGG
UUCGCCCUGCUUGGCGGUCACCCCUGCUUCCUCACUACCCAAGACAUCCACCUCG
GCGUGAACGAGAGCCUUACCGACACCGCCCGCGUGCUCUCCUCAAUGGCCGACGC
UGUGCUCGCCCGGGUGUACAAGCAGUCCGACCUUGAUACUCUCGCCAAGGAGGCC
UCCAUCCCAAUUAUCAACGGGCUCUCUGAUCUCUACCACCCUAUCCAAAUCCUCG
CGGACUACCUCACCCUCCAAGAGCACUAUAGCUCGCUCAAGGGCCUCACCCUUUC
CUGGAUUGGCGACGGCAACAACAUUCUUCACUCGAUCAUGAUGUCCGCCGCCAAG
UUCGGCAUGCAUCUCCAAGCCGCGACCCCCAAGGGCUACGAGCCUGACGCAUCCG
UGACCAAGCUCGCCGAGCAGUACGCGAAGGAAAAUGGCACCAAGCUUCUUCUCAC
CAACGACCCCCUUGAGGCCGCUCAUGGCGGCAACGUGCUCAUCACUGACACUUGG
AUCAGCAUGGGCCAGGAGGAGGAAAAGAAGAAGCGCCUUCAGGCAUUCCAGGGU
UACCAGGUCACCAUGAAAACCGCCAAAGUGGCCGCCUCCGACUGGACCUUUCUUC
ACUGUCUCCCGCGGAAGCCUGAAGAAGUGGAUGACGAAGUGUUUUACUCCCCUC
GGUCACUCGUGUUCCCGGAAGCAGAAAACAGGAAGUGGACCAUUAUGGCGGUCA
UGGUGUCCCUCCUCACCGACUACAGCCCGCAGCUUCAGAAACCCAAGUUCUAGCU
CGAGCUAGUGACUGACUAGGAUCUGGUUACCACUAAACCAGCCUCAAGAACACCC
GAAUGGAGUCUCUAAGCUACAUAAUACCAACUUACACUUACAAAAUGUUGUCCC
CCAAAAUGUAGCCAUUCGUAUCUGCUCCUAAUAAAAAGAAAGUUUCUUCACAUU
CUAG
>mARM726 (SEQ ID NO: 57)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAAUCUCCGCAUCCUCCUUAA
CAACGCAGCGUUUAGAAACGGUCACAACUUCAUGGUCCGGAACUUCCGCUGUGG
ACAGCCGCUUCAAAACAAGGUCCAGCUGAAGGGUCGGGACCUUCUGACCCUGAA
GAACUUUACUGGAGAAGAGAUCAAGUACAUGCUUUGGCUGUCCGCGGACUUGAA
GUUCCGCAUUAAGCAGAAGGGAGAAUACCUUCCGCUGCUCCAAGGAAAGAGCCU
GGGAAUGAUCUUUGAGAAGCGCUCAACCAGGACCCGCCUUUCUACUGAAACUGG
AUUCGCGCUGCUGGGUGGUCACCCUUGCUUCCUGACGACCCAGGACAUUCACCUC
GGAGUGAACGAGUCCCUCACUGAUACCGCCAGAGUGUUAUCGAGCAUGGCAGAU
GCCGUGCUGGCUAGGGUGUACAAACAGUCCGAUCUGGACACCCUGGCCAAGGAG
GCAUCAAUUCCUAUUAUCAACGGACUUAGUGACCUCUACCAUCCGAUUCAAAUCC
UGGCCGAUUACCUCACCCUGCAAGAACACUACAGCUCCCUGAAGGGUCUGACAUU
GUCCUGGAUCGGAGAUGGAAACAACAUUCUCCAUUCCAUCAUGAUGUCCGCGGC
CAAGUUCGGAAUGCAUCUCCAAGCCGCCACGCCGAAAGGAUACGAGCCGGACGCU
UCCGUGACUAAGCUCGCCGAGCAGUACGCUAAGGAGAACGGAACCAAGCUUCUG
CUGACUAACGACCCGCUAGAAGCCGCCCACGGUGGAAACGUGCUUAUUACUGACA
CCUGGAUCUCCAUGGGACAGGAAGAAGAGAAAAAGAAGCGGCUGCAGGCGUUCC
AGGGAUAUCAGGUCACCAUGAAAACCGCCAAGGUCGCCGCCUCCGACUGGACCUU
CCUUCACUGCCUGCCUCGGAAGCCUGAAGAAGUGGACGACGAGGUGUUCUACUC
GCCGCGGAGCCUCGUGUUCCCUGAGGCCGAGAAUAGAAAGUGGACCAUCAUGGC
CGUGAUGGUGUCACUCCUCACCGACUACAGCCCGCAGCUUCAGAAGCCUAAGUUC
UAGCUCGAGCUAGUGACUGACUAGGAUCUGGUUACCACUAAACCAGCCUCAAGA
ACACCCGAAUGGAGUCUCUAAGCUACAUAAUACCAACUUACACUUACAAAAUGU
UGUCCCCCAAAAUGUAGCCAUUCGUAUCUGCUCCUAAUAAAAAGAAAGUUUCUU
CACAUUCUAG
>mARM727 (SEQ ID NO: 58)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAAUCUCCGCAUUCUCCUCAA
CAACGCAGCCUUUAGAAACGGCCACAACUUCAUGGUCCGGAACUUCAGAUGUGG
CCAGCCGCUUCAGAACAAGGUCCAGCUCAAGGGCCGGGACCUCCUCACCCUCAAA
AACUUUACCGGCGAAGAGAUCAAGUACAUGCUCUGGCUUUCGGCCGACCUUAAG
UUCCGCAUCAAGCAGAAGGGGGAAUACCUUCCGCUGCUUCAAGGAAAGUCCCUC
GGCAUGAUCUUUGAAAAGCGCUCGACCAGGACCCGCCUUUCCACUGAAACCGGGU
UCGCGCUUCUCGGUGGCCACCCCUGCUUCCUCACCACCCAAGACAUUCACCUCGG
AGUGAACGAAUCCCUUACCGAUACCGCAAGAGUGCUUUCGUCGAUGGCCGAUGC
CGUGCUUGCGCGGGUGUACAAGCAGUCAGAUCUCGACACUCUCGCCAAGGAGGC
GUCCAUUCCUAUUAUCAACGGCCUUUCCGACCUUUACCACCCGAUUCAGAUCCUC
GCCGAUUACCUCACCCUGCAAGAGCACUACUCGUCACUCAAGGGUCUUACCCUCU
CCUGGAUCGGCGACGGAAACAACAUCCUCCAUUCGAUCAUGAUGUCCGCCGCCAA
AUUCGGCAUGCACCUCCAAGCCGCGACCCCGAAGGGUUACGAGCCCGACGCUUCC
GUGACCAAGCUCGCCGAACAGUACGCUAAGGAAAACGGCACCAAGCUCCUCCUCA
CUAACGACCCUCUCGAAGCAGCCCAUGGGGGCAACGUGCUCAUUACUGACACUUG
GAUCUCGAUGGGCCAGGAAGAGGAGAAAAAGAAGCGGCUUCAGGCGUUCCAGGG
AUAUCAGGUCACCAUGAAAACCGCCAAGGUCGCUGCCUCGGACUGGACCUUCCUU
CACUGCCUUCCGCGCAAGCCUGAAGAGGUGGACGAUGAGGUGUUCUACUCCCCAC
GGUCCCUUGUGUUCCCCGAGGCCGAGAAUAGGAAGUGGACCAUCAUGGCCGUGA
UGGUGUCGCUCCUCACUGACUACUCCCCGCAACUUCAGAAGCCUAAGUUCUAGCU
CGAGCUAGUGACUGACUAGGAUCUGGUUACCACUAAACCAGCCUCAAGAACACCC
GAAUGGAGUCUCUAAGCUACAUAAUACCAACUUACACUUACAAAAUGUUGUCCC
CCAAAAUGUAGCCAUUCGUAUCUGCUCCUAAUAAAAAGAAAGUUUCUUCACAUU
CUAG
>mARM728 (SEQ ID NO: 59)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUGUUUAAUCUGAGAAUACUUCUAA
ACAACGCCGCCUUCCGGAAUGGCCAUAACUUUAUGGUUCGGAAUUUCCGCUGCG
GCCAGCCGCUGCAGAACAAGGUCCAGCUGAAGGGAAGAGACUUGCUGACCCUCA
AGAACUUCACCGGAGAAGAAAUCAAGUAUAUGCUGUGGCUGUCCGCCGACCUGA
AAUUCCGCAUCAAGCAGAAGGGCGAAUAUCUGCCGCUGUUGCAAGGGAAGUCCC
UGGGGAUGAUCUUCGAGAAGAGGUCCACCAGAACACGGCUUUCAACCGAAACCG
GGUUUGCACUGCUGGGUGGACACCCCUGUUUUCUGACCACUCAAGAUAUCCACCU
GGGCGUGAACGAGUCCCUUACCGACACUGCUAGGGUGUUGUCCAGCAUGGCCGA
UGCCGUCCUGGCUCGCGUGUACAAGCAGUCCGACCUGGAUACCCUGGCAAAGGAA
GCGUCCAUUCCCAUUAUCAACGGGCUGUCCGACCUGUACCAUCCGAUUCAAAUCC
UGGCGGACUACCUGACUCUGCAAGAGCAUUACAGCAGCUUGAAGGGGCUUACUC
UCUCGUGGAUCGGCGACGGGAACAACAUCCUGCACUCCAUCAUGAUGUCCGCCGC
CAAGUUCGGGAUGCAUUUGCAAGCUGCGACCCCGAAAGGUUACGAGCCCGAUGC
UAGCGUAACUAAGCUUGCCGAACAGUACGCCAAAGAGAAUGGUACAAAACUGCU
UCUGACUAACGACCCGCUGGAAGCAGCCCACGGCGGGAACGUGCUGAUAACCGAC
ACCUGGAUUUCAAUGGGGCAGGAGGAAGAGAAGAAGAAGCGACUGCAGGCGUUC
CAAGGCUAUCAGGUUACCAUGAAAACCGCCAAAGUGGCAGCCAGCGAUUGGACU
UUCCUGCACUGUCUGCCGCGGAAGCCCGAGGAAGUUGAUGACGAAGUAUUCUAC
UCACCCCGGAGCCUCGUGUUCCCCGAGGCCGAAAACCGGAAGUGGACUAUUAUGG
CCGUGAUGGUGUCGCUGUUGACCGACUACAGCCCGCAACUGCAGAAGCCGAAGU
UUUAGCUCGAGCUAGUGACUGACUAGGAUCUGGUUACCACUAAACCAGCCUCAA
GAACACCCGAAUGGAGUCUCUAAGCUACAUAAUACCAACUUACACUUACAAAAU
GUUGUCCCCCAAAAUGUAGCCAUUCGUAUCUGCUCCUAAUAAAAAGAAAGUUUC
UUCACAUUCUAG
>mARM729 (SEQ ID NO: 60)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAACCUGAGGAUCCUUUUGA
ACAACGCCGCCUUUCGCAACGGCCACAACUUUAUGGUCCGCAAUUUCCGCUGCGG
GCAGCCGCUGCAGAACAAGGUCCAGCUGAAGGGCCGGGAUCUGCUGACCCUGAA
GAACUUCACCGGGGAGGAAAUCAAGUACAUGCUUUGGCUCUCCGCCGAUCUGAA
GUUCAGAAUCAAGCAGAAGGGAGAGUACCUCCCGUUGCUGCAAGGAAAGUCACU
CGGAAUGAUUUUCGAAAAGAGAAGCACUAGGACCCGCCUCUCAACUGAAACCGG
GUUCGCGCUGCUCGGGGGCCAUCCGUGUUUCCUGACUACCCAAGACAUCCACCUG
GGAGUGAACGAGUCGCUGACCGACACCGCACGCGUGCUGUCAUCCAUGGCGGACG
CAGUGCUUGCCCGGGUGUACAAGCAGUCGGACCUGGACACUCUUGCCAAGGAGG
CAUCAAUCCCCAUCAUUAACGGACUGUCCGAUCUCUACCACCCGAUUCAGAUCCU
GGCUGACUACCUAACCCUGCAAGAGCACUACUCAAGCCUGAAGGGGCUGACCCUG
UCGUGGAUCGGGGACGGCAACAACAUUCUGCACUCCAUCAUGAUGUCGGCGGCU
AAGUUCGGGAUGCAUUUGCAAGCGGCAACUCCGAAGGGUUAUGAACCCGACGCC
UCCGUGACCAAGCUGGCCGAACAGUACGCCAAGGAAAACGGAACCAAGUUGCUG
CUGACUAAUGAUCCCCUGGAGGCGGCCCACGGGGGGAACGUGCUGAUAACCGAU
ACCUGGAUCUCCAUGGGGCAGGAAGAAGAGAAGAAAAAGCGGCUGCAGGCAUUC
CAGGGAUACCAGGUCACCAUGAAAACCGCAAAAGUGGCAGCCAGCGACUGGACU
UUCCUCCAUUGCCUGCCGCGAAAGCCGGAGGAGGUCGAUGACGAGGUGUUCUAC
UCCCCGCGGUCGCUGGUGUUCCCGGAGGCGGAAAACCGGAAGUGGACCAUUAUG
GCCGUGAUGGUGUCACUCCUGACUGACUACAGCCCGCAACUGCAGAAGCCGAAGU
UCUAGCUCGAGCUAGUGACUGACUAGGAUCUGGUUACCACUAAACCAGCCUCAA
GAACACCCGAAUGGAGUCUCUAAGCUACAUAAUACCAACUUACACUUACAAAAU
GUUGUCCCCCAAAAUGUAGCCAUUCGUAUCUGCUCCUAAUAAAAAGAAAGUUUC
UUCACAUUCUAG
>mARM1787 (SEQ ID NO: 61)
CUUAAGGGGGCGCUGCCUACGGAGGUGGCAGCCAUCUCCUUCUCGGCAUCAAGCU
UACCAUGGUGCCCCAGGCCCUGCUCUUGGUCCCGCUGCUGGUGUUCCCCCUCUGC
UUCGGCAAGUUCCCCAUCUACACCAUCCCCGACAAGCUGGGGCCGUGGAGCCCCA
UCGACAUCCACCACCUGUCCUGCCCCAACAACCUCGUGGUCGAGGACGAGGGCUG
CACCAACCUGAGCGGGUUCUCCUACAUGCUUUUCAAUCUCCGCAUCCUCCUUAAC
AACGCCGCGUUUAGAAACGGCCACAACUUCAUGGUCCGGAACUUCAGAUGUGGC
CAGCCGCUUCAAAACAAGGUCCAGCUGAAGGGCCGGGAUCUUCUGACCCUGAAG
AACUUUACUGGCGAAGAGAUCAAGUACAUGCUCUGGCUCUCCGCGGACUUGAAG
UUCCGCAUUAAGCAGAAGGGGGAAUACCUUCCGCUGCUUCAAGGAAAGAGCCUC
GGCAUGAUCUUUGAGAAGCGCUCAACCAGGACCCGCCUUUCUACUGAAACUGGG
UUCGCGCUGCUCGGUGGCCACCCCUGCUUCCUGACGACCCAGGACAUCCACCUCG
GAGUGAACGAAUCCCUCACCGAUACCGCCCGGGUGUUAUCGAGCAUGGCAGAUG
CCGUGCUGGCCAGGGUGUACAAACAGUCCGAUCUGGACACUCUGGCCAAGGAGG
CGUCAAUUCCUAUUAUCAACGGCCUUAGUGACCUCUACCAUCCGAUUCAGAUCCU
GGCCGAUUACCUCACCCUGCAAGAACACUACAGCUCCCUGAAGGGUCUGACAUUG
UCCUGGAUCGGCGACGGCAACAACAUUCUCCAUUCCAUCAUGAUGUCCGCCGCAA
AAUUCGGCAUGCAUCUUCAAGCCGCCACGCCGAAGGGUUACGAGCCCGACGCUUC
CGUGACUAAGCUCGCCGAGCAGUACGCUAAGGAGAACGGAACCAAGCUUCUGCU
GACUAACGACCCACUAGAAGCAGCCCACGGGGGCAACGUGCUUAUUACUGACACC
UGGAUCUCCAUGGGCCAGGAAGAAGAGAAAAAGAAGCGGCUGCAGGCGUUCCAG
GGAUAUCAGGUCACCAUGAAAACCGCCAAGGUCGCUGCCUCCGACUGGACCUUCC
UGCACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCCGU
GAUGGUGUCACUGCUCACCGACUACAGCCCGCAGCUUCAGAAGCCCAAGUUCUAG
AUAAGUGAAUGCAAGGCUGGCCGGAAGCCCUUGCCUGAAAGCAAGAUUUCAGCC
UGGAAGAGGGCAAAGUGGACGGGAGUGGACAGGAGUGGAUGCGAUAAGAUGUGG
UUUGAAGCUGAUGGGUGCCAGCCCUGCAUUGCUGAGUCAAUCAAUAAAGAGCUU
UCUUUUGACCCAUUCUAGAUCUAG
>mARM1788 (SEQ ID NO: 62)
AUUAUUACAUCAAAACAAAAAGCCGCCAAUGCUGUUCAACCUGCGCAUCCUGCU
GAACAACGCCGCCUUCCGCAACGGCCACAACUUCAUGGUGCGCAACUUCCGCUGC
GGCCAGCCCCUGCAGAACAAGGUGCAGCUGAAGGGCCGCGACCUGCUGACCCUGA
AGAACUUCACCGGCGAGGAGAUCAAGUACAUGCUGUGGCUGAGCGCCGACCUGA
AGUUCCGCAUCAAGCAGAAGGGCGAGUACCUGCCCCUGCUGCAGGGCAAGAGCCU
GGGCAUGAUCUUCGAGAAGCGCAGCACCCGCACCCGCCUGAGCACCGAGACAGGC
CUGGCCCUGCUGGGCGGCCACCCCUGCUUCCUGACCACCCAGGACAUCCACCUGG
GCGUGAACGAGAGCCUGACCGACACCGCCCGCGUGCUGAGCAGCAUGGCCGACGC
CGUGCUGGCCCGCGUGUACAAGCAGAGCGACCUGGACACCCUGGCCAAGGAGGCC
AGCAUCCCCAUCAUCAACGGCCUGAGCGACCUGUACCACCCCAUCCAGAUCCUGG
CCGACUACCUGACCCUGCAGGAGCACUACAGCAGCCUGAAGGGCCUGACCCUGAG
CUGGAUCGGCGACGGCAACAACAUCCUGCACAGCAUCAUGAUGAGCGCCGCCAAG
UUCGGCAUGCACCUGCAGGCCGCCACCCCCAAGGGCUACGAGCCCGACGCCAGCG
UGACCAAGCUGGCCGAGCAGUACGCCAAGGAGAACGGCACCAAGCUGCUGCUGAC
CAACGACCCCCUGGAGGCCGCCCACGGCGGCAACGUGCUGAUCACCGACACCUGG
AUCAGCAUGGGCCAGGAGGAGGAGAAGAAGAAGCGCCUGCAGGCCUUCCAGGGC
UACCAGGUGACCAUGAAGACCGCCAAGGUGGCCGCCAGCGACUGGACCUUCCUGC
ACUGCCUGCCCCGCAAGCCCGAGGAGGUGGACGACGAGGUGUUCUACAGCCCCCG
CAGCCUGGUGUUCCCCGAGGCCGAGAACCGCAAGUGGACCAUCAUGGCCGUGAUG
GUGAGCCUGCUGACCGACUACAGCCCCCAGCUGCAGAAGCCCAAGUUCUGAACGC
CGAAGCCUGCAGCCAUGCGACCCCACGCCACCCCGUGCCUCCUGCCUCCGCGCAG
CCUGCAGCGGGAGACCCUGUCCCCGCCCCAGCCGUCCUCCUGGGGUGGACCCUAG
UUUAAUAAAGAUUCACCAAGUUUCACGC
>mARM1789 (SEQ ID NO: 63)
CUUAAGGGGGCGCUGCCUACGGAGGUGGCAGCCAUCUCCUUCUCGGCAUCAAGCU
UACCAUGGUGCCCCAGGCCCUGCUCUUGGUCCCGCUGCUGGUGUUCCCCCUCUGC
UUCGGCAAGUUCCCCAUCUACACCAUCCCCGACAAGCUGGGGCCGUGGAGCCCCA
UCGACAUCCACCACCUGUCCUGCCCCAACAACCUCGUGGUCGAGGACGAGGGCUG
CACCAACCUGAGCGGGUUCUCCUACAUGCUGUUCAACCUGCGCAUCCUGCUGAAC
AACGCCGCCUUCCGCAACGGCCACAACUUCAUGGUGCGCAACUUCCGCUGCGGCC
AGCCCCUGCAGAACAAGGUGCAGCUGAAGGGCCGCGACCUGCUGACCCUGAAGAA
CUUCACCGGCGAGGAGAUCAAGUACAUGCUGUGGCUGAGCGCCGACCUGAAGUU
CCGCAUCAAGCAGAAGGGCGAGUACCUGCCCCUGCUGCAGGGCAAGAGCCUGGGC
AUGAUCUUCGAGAAGCGCAGCACCCGCACCCGCCUGAGCACCGAGACAGGCCUGG
CCCUGCUGGGCGGCCACCCCUGCUUCCUGACCACCCAGGACAUCCACCUGGGCGU
GAACGAGAGCCUGACCGACACCGCCCGCGUGCUGAGCAGCAUGGCCGACGCCGUG
CUGGCCCGCGUGUACAAGCAGAGCGACCUGGACACCCUGGCCAAGGAGGCCAGCA
UCCCCAUCAUCAACGGCCUGAGCGACCUGUACCACCCCAUCCAGAUCCUGGCCGA
CUACCUGACCCUGCAGGAGCACUACAGCAGCCUGAAGGGCCUGACCCUGAGCUGG
AUCGGCGACGGCAACAACAUCCUGCACAGCAUCAUGAUGAGCGCCGCCAAGUUCG
GCAUGCACCUGCAGGCCGCCACCCCCAAGGGCUACGAGCCCGACGCCAGCGUGAC
CAAGCUGGCCGAGCAGUACGCCAAGGAGAACGGCACCAAGCUGCUGCUGACCAAC
GACCCCCUGGAGGCCGCCCACGGCGGCAACGUGCUGAUCACCGACACCUGGAUCA
GCAUGGGCCAGGAGGAGGAGAAGAAGAAGCGCCUGCAGGCCUUCCAGGGCUACC
AGGUGACCAUGAAGACCGCCAAGGUGGCCGCCAGCGACUGGACCUUCCUGCACUG
CCUGCCCCGCAAGCCCGAGGAGGUGGACGACGAGGUGUUCUACAGCCCCCGCAGC
CUGGUGUUCCCCGAGGCCGAGAACCGCAAGUGGACCAUCAUGGCCGUGAUGGUG
AGCCUGCUGACCGACUACAGCCCCCAGCUGCAGAAGCCCAAGUUCUGAAUAAGUG
AUGCAAGGCUGGCCGGAAGCCCUUGCCUGAAAGCAAGAUUUCAGCCUGGAAGAG
GGCAAAGUGGACGGGAGUGGACAGGAGUGGAUGCGAUAAGAUGUGGUUUGAAGC
UGAUGGGUGCCAGCCCUGCAUUGCUGAGUCAAUCAAUAAAGAGCUUUCUUUUGA
CCCAUUCUAGAUCUAG
>mARM1790 (SEQ ID NO: 64)
AUUAUUACAUCAAAACAAAAAGCCGCCAAUGCUUUUCAAUCUCCGCAUCCUCCUU
AACAACGCCGCGUUUAGAAACGGCCACAACUUCAUGGUCCGGAACUUCAGAUGU
GGCCAGCCGCUUCAAAACAAGGUCCAGCUGAAGGGCCGGGAUCUUCUGACCCUGA
AGAACUUUACUGGCGAAGAGAUCAAGUACAUGCUCUGGCUCUCCGCGGACUUGA
AGUUCCGCAUUAAGCAGAAGGGGGAAUACCUUCCGCUGCUUCAAGGAAAGAGCC
UCGGCAUGAUCUUUGAGAAGCGCUCAACCAGGACCCGCCUUUCUACUGAAACUG
GGUUCGCGCUGCUCGGUGGCCACCCCUGCUUCCUGACGACCCAGGACAUCCACCU
CGGAGUGAACGAAUCCCUCACCGAUACCGCCCGGGUGUUAUCGAGCAUGGCAGA
UGCCGUGCUGGCCAGGGUGUACAAACAGUCCGAUCUGGACACUCUGGCCAAGGA
GGCGUCAAUUCCUAUUAUCAACGGCCUUAGUGACCUCUACCAUCCGAUUCAGAUC
CUGGCCGAUUACCUCACCCUGCAAGAACACUACAGCUCCCUGAAGGGUCUGACAU
UGUCCUGGAUCGGCGACGGCAACAACAUUCUCCAUUCCAUCAUGAUGUCCGCCGC
AAAAUUCGGCAUGCAUCUUCAAGCCGCCACGCCGAAGGGUUACGAGCCCGACGCU
UCCGUGACUAAGCUCGCCGAGCAGUACGCUAAGGAGAACGGAACCAAGCUUCUG
CUGACUAACGACCCACUAGAAGCAGCCCACGGGGGCAACGUGCUUAUUACUGACA
CCUGGAUCUCCAUGGGCCAGGAAGAAGAGAAAAAGAAGCGGCUGCAGGCGUUCC
AGGGAUAUCAGGUCACCAUGAAAACCGCCAAGGUCGCUGCCUCCGACUGGACCUU
CCUGCACUGCCUGCCUCGCAAGCCUGAAGAAGUGGACGACGAGGUGUUCUACUCG
CCACGGAGCCUCGUGUUCCCCGAGGCCGAGAAUAGAAAGUGGACCAUCAUGGCCG
UGAUGGUGUCACUGCUCACCGACUACAGCCCGCAGCUUCAGAAGCCCAAGUUCUA
GACGCCGAAGCCUGCAGCCAUGCGACCCCACGCCACCCCGUGCCUCCUGCCUCCG
CGCAGCCUGCAGCGGGAGACCCUGUCCCCGCCCCAGCCGUCCUCCUGGGGUGGAC
CCUAGUUUAAUAAAGAUUCACCAAGUUUCACGC
>mARM1791 (SEQ ID NO: 65)
CUUAAGGGGGCGCUGCCUACGGAGGUGGCAGCCAUCUCCUUCUCGGCAUCAAGCU
UACCAUGGUGCCCCAGGCCCUGCUCUUGGUCCCGCUGCUGGUGUUCCCCCUCUGC
UUCGGCAAGUUCCCCAUCUACACCAUCCCCGACAAGCUGGGGCCGUGGAGCCCCA
UCGACAUCCACCACCUGUCCUGCCCCAACAACCUCGUGGUCGAGGACGAGGGCUG
CACCAACCUGAGCGGGUUCUCCUACAUGCUUUUCAACCUGAGAAUCCUCUUGAAC
AAUGCUGCUUUUCGGAAUGGCCACAACUUUAUGGUUCGGAACUUCCGUUGCGGC
CAGCCUUUACAAAACAAGGUCCAGCUGAAGGGCCGGGAUUUGCUCACACUAAAG
AACUUUACUGGAGAAGAGAUCAAGUACAUGCUAUGGCUGUCGGCCGACCUGAAG
UUCCGUAUCAAGCAGAAGGGAGAAUACCUUCCGCUGCUUCAAGGAAAGAGCCUC
GGCAUGAUCUUUGAGAAGCGCUCAACCAGGACCCGCCUUUCUACUGAAACUGGG
UUCGCGCUGCUCGGUGGCCACCCCUGCUUCCUGACGACCCAGGACAUCCACCUCG
GAGUGAACGAAUCCCUCACCGAUACCGCCCGGGUGUUAUCGAGCAUGGCAGAUG
CCGUGCUGGCCAGGGUGUACAAACAGUCCGAUCUCGAUACCUUGGCAAAGGAGG
CUUCCAUUCCCAUCAUCAACGGCCUGAGCGACCUGUACCACCCAAUCCAAAUCCU
GGCUGACUACCUGACCCUGCAAGAGCACUACAGCAGCCUGAAGGGUCUGACCCUG
UCAUGGAUUGGCGAUGGAAACAAUAUUCUGCACUCCAUCAUGAUGUCCGCCGCG
AAGUUCGGAAUGCAUCUGCAAGCCGCCACUCCAAAAGGAUACGAACCGGAUGCA
UCCGUGACCAAGUUGGCGGAACAGUACGCGAAGGAGAACGGAACCAAGCUCCUG
CUGACUAACGACCCGCUCGAGGCUGCGCAUGGGGGUAACGUGCUGAUUACGGAC
ACCUGGAUCUCCAUGGGGCAGGAGGAAGAGAAGAAGAAGAGACUGCAGGCAUUC
CAGGGGUACCAGGUCACCAUGAAAACCGCAAAAGUGGCAGCUUCGGACUGGACU
UUCCUGCAUUGCCUGCCGAGGAAGCCGGAGGAAGUCGACGACGAAGUGUUCUAC
UCGCCUCGGUCCCUGGUGUUCCCCGAGGCCGAAAACCGGAAGUGGACCAUCAUGG
CCGUGAUGGUGUCCUUGCUGACUGACUAUAGCCCGCAGCUGCAGAAGCCUAAGU
UCUAGAUAAGUGAUGCAAGGCUGGCCGGAAGCCCUUGCCUGAAAGCAAGAUUUC
AGCCUGGAAGAGGGCAAAGUGGACGGGAGUGGACAGGAGUGGAUGCGAUAAGAU
GUGGUUUGAAGCUGAUGGGUGCCAGCCCUGCAUUGCUGAGUCAAUCAAUAAAGA
GCUUUCUUUUGACCCAUUCUAGAUCUAG
>mARM1792 (SEQ ID NO: 66)
UGAGUGUCGUACAGCCUCCAGGCCCCCCCCUCCCGGGAGAGCCAUAGUGGUCUGC
GGAACCGGUGAGUACACCGGAAUUGCCGGGAAGACUGGGUCCUUUCUUGGAUAA
ACCCACUCUAUGCCCGGCCAUUUGGGCGUGCCCCCGCAAGACUGCUAGCCGAGUA
GUGUUGGGUUGCGAUGCUGUUCAACCUGCGCAUCCUGCUGAACAACGCCGCCUUC
CGCAACGGCCACAACUUCAUGGUGCGCAACUUCCGCUGCGGCCAGCCCCUGCAGA
ACAAGGUGCAGCUGAAGGGCCGCGACCUGCUGACCCUGAAGAACUUCACCGGCGA
GGAGAUCAAGUACAUGCUGUGGCUGAGCGCCGACCUGAAGUUCCGCAUCAAGCA
GAAGGGCGAGUACCUGCCCCUGCUGCAGGGCAAGAGCCUGGGCAUGAUCUUCGA
GAAGCGCAGCACCCGCACCCGCCUGAGCACCGAGACAGGCCUGGCCCUGCUGGGC
GGCCACCCCUGCUUCCUGACCACCCAGGACAUCCACCUGGGCGUGAACGAGAGCC
UGACCGACACCGCCCGCGUGCUGAGCAGCAUGGCCGACGCCGUGCUGGCCCGCGU
GUACAAGCAGAGCGACCUGGACACCCUGGCCAAGGAGGCCAGCAUCCCCAUCAUC
AACGGCCUGAGCGACCUGUACCACCCCAUCCAGAUCCUGGCCGACUACCUGACCC
UGCAGGAGCACUACAGCAGCCUGAAGGGCCUGACCCUGAGCUGGAUCGGCGACG
GCAACAACAUCCUGCACAGCAUCAUGAUGAGCGCCGCCAAGUUCGGCAUGCACCU
GCAGGCCGCCACCCCCAAGGGCUACGAGCCCGACGCCAGCGUGACCAAGCUGGCC
GAGCAGUACGCCAAGGAGAACGGCACCAAGCUGCUGCUGACCAACGACCCCCUGG
AGGCCGCCCACGGCGGCAACGUGCUGAUCACCGACACCUGGAUCAGCAUGGGCCA
GGAGGAGGAGAAGAAGAAGCGCCUGCAGGCCUUCCAGGGCUACCAGGUGACCAU
GAAGACCGCCAAGGUGGCCGCCAGCGACUGGACCUUCCUGCACUGCCUGCCCCGC
AAGCCCGAGGAGGUGGACGACGAGGUGUUCUACAGCCCCCGCAGCCUGGUGUUCC
CCGAGGCCGAGAACCGCAAGUGGACCAUCAUGGCCGUGAUGGUGAGCCUGCUGA
CCGACUACAGCCCCCAGCUGCAGAAGCCCAAGUUCUGAAUAAGUGAUAGAGCGGC
AAACCCUAGCUACACUCCAUAGCUAGUUUCUUUUUUUUUUGUUUUUUUUUUUUU
UUUUUUUUUUUUUUUUUUUUUUUUUUCCUUUCUUUUCCUUCUUUUUUUCCUCUU
UUCUUGGUGGCUCCAUCUUAGCCCUAGUCACGGCUAGCUGUGAAAGGUCCGUGA
GCCGCAUGACUGCAGAGAGUGCCGUAACUGGCCUCUCUGCAGAUCAUGUUCUAG
>mARM1793 (SEQ ID NO: 67)
AUUAUUACAUCAAAACAAAAAGCCGCCAAUGCUUUUCAAUCUCCGCAUCCUCCUU
AACAACGCCGCGUUUAGAAACGGCCACAACUUCAUGGUCCGGAACUUCAGAUGU
GGCCAGCCGCUUCAAAACAAGGUCCAGCUGAAGGGCCGGGAUCUUCUGACCCUGA
AGAACUUUACUGGCGAAGAGAUCAAGUACAUGCUCUGGCUCUCCGCGGACUUGA
AGUUCCGCAUUAAGCAGAAGGGGGAAUACCUUCCGCUGCUUCAAGGAAAGAGCC
UCGGCAUGAUCUUUGAGAAGCGCUCAACCAGGACCCGCCUUUCUACUGAAACUG
GGUUCGCGCUGCUCGGUGGCCACCCCUGCUUCCUGACGACCCAGGACAUCCACCU
CGGAGUGAACGAAUCCCUCACCGAUACCGCCCGGGUGUUAUCGAGCAUGGCAGA
UGCCGUGCUGGCCAGGGUGUACAAACAGUCCGAUCUGGACACUCUGGCCAAGGA
GGCGUCAAUUCCUAUUAUCAACGGCCUUAGUGACCUCUACCAUCCGAUUCAGAUC
CUGGCCGAUUACCUCACCCUGCAAGAACACUACAGCUCCCUGAAGGGUCUGACAU
UGUCCUGGAUCGGCGACGGCAACAACAUUCUCCAUUCCAUCAUGAUGUCCGCCGC
AAAAUUCGGCAUGCAUCUUCAAGCCGCCACGCCGAAGGGUUACGAGCCCGACGCU
UCCGUGACUAAGCUCGCCGAGCAGUACGCUAAGGAGAACGGAACCAAGCUUCUG
CUGACUAACGACCCACUAGAAGCAGCCCACGGGGGCAACGUGCUUAUUACUGACA
CCUGGAUCUCCAUGGGCCAGGAAGAAGAGAAAAAGAAGCGGCUGCAGGCGUUCC
AGGGAUAUCAGGUCACCAUGAAAACCGCCAAGGUCGCUGCCUCCGACUGGACCUU
CCUGCACUGCCUGCCUCGCAAGCCUGAAGAAGUGGACGACGAGGUGUUCUACUCG
CCACGGAGCCUCGUGUUCCCCGAGGCCGAGAAUAGAAAGUGGACCAUCAUGGCCG
UGAUGGUGUCACUGCUCACCGACUACAGCCCGCAGCUUCAGAAGCCCAAGUUCUA
GGCUGGAGCCUCGGUAGCCGUUCCUCCUGCCCGCUGGGCCUCCCAACGGGCCCUC
CUCCCCUCCUUGCACCGGCCCUUCCUGGUCUUUGAAUAAAGUCUGAGUGGGCAGC
>mARM1794 (SEQ ID NO: 68)
AUUAUUACAUCAAAACAAAAAGCCGCCAAUGCUGUUCAACCUGCGCAUCCUGCU
GAACAACGCCGCCUUCCGCAACGGCCACAACUUCAUGGUGCGCAACUUCCGCUGC
GGCCAGCCCCUGCAGAACAAGGUGCAGCUGAAGGGCCGCGACCUGCUGACCCUGA
AGAACUUCACCGGCGAGGAGAUCAAGUACAUGCUGUGGCUGAGCGCCGACCUGA
AGUUCCGCAUCAAGCAGAAGGGCGAGUACCUGCCCCUGCUGCAGGGCAAGAGCCU
GGGCAUGAUCUUCGAGAAGCGCAGCACCCGCACCCGCCUGAGCACCGAGACAGGC
CUGGCCCUGCUGGGCGGCCACCCCUGCUUCCUGACCACCCAGGACAUCCACCUGG
GCGUGAACGAGAGCCUGACCGACACCGCCCGCGUGCUGAGCAGCAUGGCCGACGC
CGUGCUGGCCCGCGUGUACAAGCAGAGCGACCUGGACACCCUGGCCAAGGAGGCC
AGCAUCCCCAUCAUCAACGGCCUGAGCGACCUGUACCACCCCAUCCAGAUCCUGG
CCGACUACCUGACCCUGCAGGAGCACUACAGCAGCCUGAAGGGCCUGACCCUGAG
CUGGAUCGGCGACGGCAACAACAUCCUGCACAGCAUCAUGAUGAGCGCCGCCAAG
UUCGGCAUGCACCUGCAGGCCGCCACCCCCAAGGGCUACGAGCCCGACGCCAGCG
UGACCAAGCUGGCCGAGCAGUACGCCAAGGAGAACGGCACCAAGCUGCUGCUGAC
CAACGACCCCCUGGAGGCCGCCCACGGCGGCAACGUGCUGAUCACCGACACCUGG
AUCAGCAUGGGCCAGGAGGAGGAGAAGAAGAAGCGCCUGCAGGCCUUCCAGGGC
UACCAGGUGACCAUGAAGACCGCCAAGGUGGCCGCCAGCGACUGGACCUUCCUGC
ACUGCCUGCCCCGCAAGCCCGAGGAGGUGGACGACGAGGUGUUCUACAGCCCCCG
CAGCCUGGUGUUCCCCGAGGCCGAGAACCGCAAGUGGACCAUCAUGGCCGUGAUG
GUGAGCCUGCUGACCGACUACAGCCCCCAGCUGCAGAAGCCCAAGUUCUGAGCUG
GAGCCUCGGUAGCCGUUCCUCCUGCCCGCUGGGCCUCCCAACGGGCCCUCCUCCC
CUCCUUGCACCGGCCCUUCCUGGUCUUUGAAUAAAGUCUGAGUGGGCAGC
>mARM1795 (SEQ ID NO: 69)
AUUAUUACAUCAAAACAAAAAGCCGCCAAUGCUUUUCAACCUGAGAAUCCUCUU
GAACAAUGCUGCUUUUCGGAAUGGCCACAACUUUAUGGUUCGGAACUUCCGUUG
CGGCCAGCCUUUACAAAACAAGGUCCAGCUGAAGGGCCGGGAUUUGCUCACACU
AAAGAACUUUACUGGAGAAGAGAUCAAGUACAUGCUAUGGCUGUCGGCCGACCU
GAAGUUCCGUAUCAAGCAGAAGGGAGAAUACCUUCCGCUGCUUCAAGGAAAGAG
CCUCGGCAUGAUCUUUGAGAAGCGCUCAACCAGGACCCGCCUUUCUACUGAAACU
GGGUUCGCGCUGCUCGGUGGCCACCCCUGCUUCCUGACGACCCAGGACAUCCACC
UCGGAGUGAACGAAUCCCUCACCGAUACCGCCCGGGUGUUAUCGAGCAUGGCAG
AUGCCGUGCUGGCCAGGGUGUACAAACAGUCCGAUCUCGAUACCUUGGCAAAGG
AGGCUUCCAUUCCCAUCAUCAACGGCCUGAGCGACCUGUACCACCCAAUCCAAAU
CCUGGCUGACUACCUGACCCUGCAAGAGCACUACAGCAGCCUGAAGGGUCUGACC
CUGUCAUGGAUUGGCGAUGGAAACAAUAUUCUGCACUCCAUCAUGAUGUCCGCC
GCGAAGUUCGGAAUGCAUCUGCAAGCCGCCACUCCAAAAGGAUACGAACCGGAU
GCAUCCGUGACCAAGUUGGCGGAACAGUACGCGAAGGAGAACGGAACCAAGCUC
CUGCUGACUAACGACCCGCUCGAGGCUGCGCAUGGGGGUAACGUGCUGAUUACG
GACACCUGGAUCUCCAUGGGGCAGGAGGAAGAGAAGAAGAAGAGACUGCAGGCA
UUCCAGGGGUACCAGGUCACCAUGAAAACCGCAAAAGUGGCAGCUUCGGACUGG
ACUUUCCUGCAUUGCCUGCCGAGGAAGCCGGAGGAAGUCGACGACGAAGUGUUC
UACUCGCCUCGGUCCCUGGUGUUCCCCGAGGCCGAAAACCGGAAGUGGACCAUCA
UGGCCGUGAUGGUGUCCUUGCUGACUGACUAUAGCCCGCAGCUGCAGAAGCCUA
AGUUCUAGGCUGGAGCCUCGGUAGCCGUUCCUCCUGCCCGCUGGGCCUCCCAACG
GGCCCUCCUCCCCUCCUUGCACCGGCCCUUCCUGGUCUUUGAAUAAAGUCUGAGU
GGGCAGC
>mARM1796 (SEQ ID NO: 70)
AAUUAUUGGUUAAAGAAGUAUAUUAGUGCUAAUUUCCCUCCGUUUGUCCUAGCU
UUUCUCUUCUGUCAACCCCACACGCCUUUGGCACAAUGCUUUUCAAUCUCCGCAU
CCUCCUUAACAACGCCGCGUUUAGAAACGGCCACAACUUCAUGGUCCGGAACUUC
AGAUGUGGCCAGCCGCUUCAAAACAAGGUCCAGCUGAAGGGCCGGGAUCUUCUG
ACCCUGAAGAACUUUACUGGCGAAGAGAUCAAGUACAUGCUCUGGCUCUCCGCG
GACUUGAAGUUCCGCAUUAAGCAGAAGGGGGAAUACCUUCCGCUGCUUCAAGGA
AAGAGCCUCGGCAUGAUCUUUGAGAAGCGCUCAACCAGGACCCGCCUUUCUACUG
AAACUGGGUUCGCGCUGCUCGGUGGCCACCCCUGCUUCCUGACGACCCAGGACAU
CCACCUCGGAGUGAACGAAUCCCUCACCGAUACCGCCCGGGUGUUAUCGAGCAUG
GCAGAUGCCGUGCUGGCCAGGGUGUACAAACAGUCCGAUCUGGACACUCUGGCC
AAGGAGGCGUCAAUUCCUAUUAUCAACGGCCUUAGUGACCUCUACCAUCCGAUU
CAGAUCCUGGCCGAUUACCUCACCCUGCAAGAACACUACAGCUCCCUGAAGGGUC
UGACAUUGUCCUGGAUCGGCGACGGCAACAACAUUCUCCAUUCCAUCAUGAUGU
CCGCCGCAAAAUUCGGCAUGCAUCUUCAAGCCGCCACGCCGAAGGGUUACGAGCC
CGACGCUUCCGUGACUAAGCUCGCCGAGCAGUACGCUAAGGAGAACGGAACCAA
GCUUCUGCUGACUAACGACCCACUAGAAGCAGCCCACGGGGGCAACGUGCUUAUU
ACUGACACCUGGAUCUCCAUGGGCCAGGAAGAAGAGAAAAAGAAGCGGCUGCAG
GCGUUCCAGGGAUAUCAGGUCACCAUGAAAACCGCCAAGGUCGCUGCCUCCGACU
GGACCUUCCUGCACUGCCUGCCUCGCAAGCCUGAAGAAGUGGACGACGAGGUGU
UCUACUCGCCACGGAGCCUCGUGUUCCCCGAGGCCGAGAAUAGAAAGUGGACCAU
CAUGGCCGUGAUGGUGUCACUGCUCACCGACUACAGCCCGCAGCUUCAGAAGCCC
AAGUUCUAGCUCGAGACACAUCACAACCACAACCUUCUCAGGCUACCCUGAGAAA
AAAAGACAUGAAGACUCAGGACUCAUCUUUUCUGUUGGUGUAAAAUCAACACCC
UAAGGAACACAAAUUUCUUUAAACAUUUGACUUCUUGUCUCUGUGCUGCAAUUA
AUAAAAAAUGGAAAGAAUCUAUCUAG
>mARM1797 (SEQ ID NO: 71)
AAUUAUUGGUUAAAGAAGUAUAUUAGUGCUAAUUUCCCUCCGUUUGUCCUAGCU
UUUCUCUUCUGUCAACCCCACACGCCUUUGGCACAAUGCUGUUCAACCUGCGCAU
CCUGCUGAACAACGCCGCCUUCCGCAACGGCCACAACUUCAUGGUGCGCAACUUC
CGCUGCGGCCAGCCCCUGCAGAACAAGGUGCAGCUGAAGGGCCGCGACCUGCUGA
CCCUGAAGAACUUCACCGGCGAGGAGAUCAAGUACAUGCUGUGGCUGAGCGCCG
ACCUGAAGUUCCGCAUCAAGCAGAAGGGCGAGUACCUGCCCCUGCUGCAGGGCAA
GAGCCUGGGCAUGAUCUUCGAGAAGCGCAGCACCCGCACCCGCCUGAGCACCGAG
ACAGGCCUGGCCCUGCUGGGCGGCCACCCCUGCUUCCUGACCACCCAGGACAUCC
ACCUGGGCGUGAACGAGAGCCUGACCGACACCGCCCGCGUGCUGAGCAGCAUGGC
CGACGCCGUGCUGGCCCGCGUGUACAAGCAGAGCGACCUGGACACCCUGGCCAAG
GAGGCCAGCAUCCCCAUCAUCAACGGCCUGAGCGACCUGUACCACCCCAUCCAGA
UCCUGGCCGACUACCUGACCCUGCAGGAGCACUACAGCAGCCUGAAGGGCCUGAC
CCUGAGCUGGAUCGGCGACGGCAACAACAUCCUGCACAGCAUCAUGAUGAGCGCC
GCCAAGUUCGGCAUGCACCUGCAGGCCGCCACCCCCAAGGGCUACGAGCCCGACG
CCAGCGUGACCAAGCUGGCCGAGCAGUACGCCAAGGAGAACGGCACCAAGCUGCU
GCUGACCAACGACCCCCUGGAGGCCGCCCACGGCGGCAACGUGCUGAUCACCGAC
ACCUGGAUCAGCAUGGGCCAGGAGGAGGAGAAGAAGAAGCGCCUGCAGGCCUUC
CAGGGCUACCAGGUGACCAUGAAGACCGCCAAGGUGGCCGCCAGCGACUGGACCU
UCCUGCACUGCCUGCCCCGCAAGCCCGAGGAGGUGGACGACGAGGUGUUCUACAG
CCCCCGCAGCCUGGUGUUCCCCGAGGCCGAGAACCGCAAGUGGACCAUCAUGGCC
GUGAUGGUGAGCCUGCUGACCGACUACAGCCCCCAGCUGCAGAAGCCCAAGUUCU
GACUCGAGACACAUCACAACCACAACCUUCUCAGGCUACCCUGAGAAAAAAAGAC
AUGAAGACUCAGGACUCAUCUUUUCUGUUGGUGUAAAAUCAACACCCUAAGGAA
CACAAAUUUCUUUAAACAUUUGACUUCUUGUCUCUGUGCUGCAAUUAAUAAAAA
AUGGAAAGAAUCUAUCUAG
>mARM1798 (SEQ ID NO: 72)
AAUUAUUGGUUAAAGAAGUAUAUUAGUGCUAAUUUCCCUCCGUUUGUCCUAGCU
UUUCUCUUCUGUCAACCCCACACGCCUUUGGCACAAUGCUUUUCAACCUGAGAAU
CCUCUUGAACAAUGCUGCUUUUCGGAAUGGCCACAACUUUAUGGUUCGGAACUU
CCGUUGCGGCCAGCCUUUACAAAACAAGGUCCAGCUGAAGGGCCGGGAUUUGCU
CACACUAAAGAACUUUACUGGAGAAGAGAUCAAGUACAUGCUAUGGCUGUCGGC
CGACCUGAAGUUCCGUAUCAAGCAGAAGGGAGAAUACCUUCCGCUGCUUCAAGG
AAAGAGCCUCGGCAUGAUCUUUGAGAAGCGCUCAACCAGGACCCGCCUUUCUACU
GAAACUGGGUUCGCGCUGCUCGGUGGCCACCCCUGCUUCCUGACGACCCAGGACA
UCCACCUCGGAGUGAACGAAUCCCUCACCGAUACCGCCCGGGUGUUAUCGAGCAU
GGCAGAUGCCGUGCUGGCCAGGGUGUACAAACAGUCCGAUCUCGAUACCUUGGC
AAAGGAGGCUUCCAUUCCCAUCAUCAACGGCCUGAGCGACCUGUACCACCCAAUC
CAAAUCCUGGCUGACUACCUGACCCUGCAAGAGCACUACAGCAGCCUGAAGGGUC
UGACCCUGUCAUGGAUUGGCGAUGGAAACAAUAUUCUGCACUCCAUCAUGAUGU
CCGCCGCGAAGUUCGGAAUGCAUCUGCAAGCCGCCACUCCAAAAGGAUACGAACC
GGAUGCAUCCGUGACCAAGUUGGCGGAACAGUACGCGAAGGAGAACGGAACCAA
GCUCCUGCUGACUAACGACCCGCUCGAGGCUGCGCAUGGGGGUAACGUGCUGAU
UACGGACACCUGGAUCUCCAUGGGGCAGGAGGAAGAGAAGAAGAAGAGACUGCA
GGCAUUCCAGGGGUACCAGGUCACCAUGAAAACCGCAAAAGUGGCAGCUUCGGA
CUGGACUUUCCUGCAUUGCCUGCCGAGGAAGCCGGAGGAAGUCGACGACGAAGU
GUUCUACUCGCCUCGGUCCCUGGUGUUCCCCGAGGCCGAAAACCGGAAGUGGACC
AUCAUGGCCGUGAUGGUGUCCUUGCUGACUGACUAUAGCCCGCAGCUGCAGAAG
CCUAAGUUCUAGCUCGAGACACAUCACAACCACAACCUUCUCAGGCUACCCUGAG
AAAAAAAGACAUGAAGACUCAGGACUCAUCUUUUCUGUUGGUGUAAAAUCAACA
CCCUAAGGAACACAAAUUUCUUUAAACAUUUGACUUCUUGUCUCUGUGCUGCAA
UUAAUAAAAAAUGGAAAGAAUCUAUCUAG
>mARM1799 (SEQ ID NO: 73)
AUUAUUACAUCAAAACAAAAAGCCGCCACCAUGCUGUUCAACCUGCGCAUCCUGC
UGAACAACGCCGCCUUCCGCAACGGCCACAACUUCAUGGUGCGCAACUUCCGCUG
CGGCCAGCCCCUGCAGAACAAGGUGCAGCUGAAGGGCCGCGACCUGCUGACCCUG
AAGAACUUCACCGGCGAGGAGAUCAAGUACAUGCUGUGGCUGAGCGCCGACCUG
AAGUUCCGCAUCAAGCAGAAGGGCGAGUACCUGCCCCUGCUGCAGGGCAAGAGCC
UGGGCAUGAUCUUCGAGAAGCGCAGCACCCGCACCCGCCUGAGCACCGAGACAGG
CUUCGCCCUGCUGGGCGGCCACCCCUGCUUCCUGACCACCCAGGACAUCCACCUG
GGCGUGAACGAGAGCCUGACCGACACCGCCCGCGUGCUGAGCAGCAUGGCCGACG
CCGUGCUGGCCCGCGUGUACAAGCAGAGCGACCUGGACACCCUGGCCAAGGAGGC
CAGCAUCCCCAUCAUCAACGGCCUGAGCGACCUGUACCACCCCAUCCAGAUCCUG
GCCGACUACCUGACCCUGCAGGAGCACUACAGCAGCCUGAAGGGCCUGACCCUGA
GCUGGAUCGGCGACGGCAACAACAUCCUGCACAGCAUCAUGAUGAGCGCCGCCAA
GUUCGGCAUGCACCUGCAGGCCGCCACCCCCAAGGGCUACGAGCCCGACGCCAGC
GUGACCAAGCUGGCCGAGCAGUACGCCAAGGAGAACGGCACCAAGCUGCUGCUG
ACCAACGACCCCCUGGAGGCCGCCCACGGCGGCAACGUGCUGAUCACCGACACCU
GGAUCAGCAUGGGCCAGGAGGAGGAGAAGAAGAAGCGCCUGCAGGCCUUCCAGG
GCUACCAGGUGACCAUGAAGACCGCCAAGGUGGCCGCCAGCGACUGGACCUUCCU
GCACUGCCUGCCCCGCAAGCCCGAGGAGGUGGACGACGAGGUGUUCUACAGCCCC
CGCAGCCUGGUGUUCCCCGAGGCCGAGAACCGCAAGUGGACCAUCAUGGCCGUGA
UGGUGAGCCUGCUGACCGACUACAGCCCCCAGCUGCAGAAGCCCAAGUUCUGAGG
UCUCUAGUAAUGAGCUGGAGCCUCGGUAGCCGUUCCUCCUGCCCGCUGGGCCUCC
CAACGGGCCCUCCUCCCCUCCUUGCACCGGCCCUUCCUGGUCUUUGAAUAAAGUC
UGAGUGGGCAUCUAG
>mARM1800 (SEQ ID NO: 74)
AAUUAUUGGUUAAAGAAGUAUAUUAGUGCUAAUUUCCCUCCGUUUGUCCUAGCU
UUUCUCUUCUGUCAACCCCACACGCCUUUGGCACAAUGCUGUUCAACCUGCGCAU
CCUGCUGAACAACGCCGCCUUCCGCAACGGCCACAACUUCAUGGUGCGCAACUUC
CGCUGCGGCCAGCCCCUGCAGAACAAGGUGCAGCUGAAGGGCCGCGACCUGCUGA
CCCUGAAGAACUUCACCGGCGAGGAGAUCAAGUACAUGCUGUGGCUGAGCGCCG
ACCUGAAGUUCCGCAUCAAGCAGAAGGGCGAGUACCUGCCCCUGCUGCAGGGCAA
GAGCCUGGGCAUGAUCUUCGAGAAGCGCAGCACCCGCACCCGCCUGAGCACCGAG
ACAGGCUUCGCCCUGCUGGGCGGCCACCCCUGCUUCCUGACCACCCAGGACAUCC
ACCUGGGCGUGAACGAGAGCCUGACCGACACCGCCCGCGUGCUGAGCAGCAUGGC
CGACGCCGUGCUGGCCCGCGUGUACAAGCAGAGCGACCUGGACACCCUGGCCAAG
GAGGCCAGCAUCCCCAUCAUCAACGGCCUGAGCGACCUGUACCACCCCAUCCAGA
UCCUGGCCGACUACCUGACCCUGCAGGAGCACUACAGCAGCCUGAAGGGCCUGAC
CCUGAGCUGGAUCGGCGACGGCAACAACAUCCUGCACAGCAUCAUGAUGAGCGCC
GCCAAGUUCGGCAUGCACCUGCAGGCCGCCACCCCCAAGGGCUACGAGCCCGACG
CCAGCGUGACCAAGCUGGCCGAGCAGUACGCCAAGGAGAACGGCACCAAGCUGCU
GCUGACCAACGACCCCCUGGAGGCCGCCCACGGCGGCAACGUGCUGAUCACCGAC
ACCUGGAUCAGCAUGGGCCAGGAGGAGGAGAAGAAGAAGCGCCUGCAGGCCUUC
CAGGGCUACCAGGUGACCAUGAAGACCGCCAAGGUGGCCGCCAGCGACUGGACCU
UCCUGCACUGCCUGCCCCGCAAGCCCGAGGAGGUGGACGACGAGGUGUUCUACAG
CCCCCGCAGCCUGGUGUUCCCCGAGGCCGAGAACCGCAAGUGGACCAUCAUGGCC
GUGAUGGUGAGCCUGCUGACCGACUACAGCCCCCAGCUGCAGAAGCCCAAGUUCU
GACUCGAGACACAUCACAACCACAACCUUCUCAGGCUACCCUGAGAAAAAAAGAC
AUGAAGACUCAGGACUCAUCUUUUCUGUUGGUGUAAAAUCAACACCCUAAGGAA
CACAAAUUUCUUUAAACAUUUGACUUCUUGUCUCUGUGCUGCAAUUAAUAAAAA
AUGGAAAGAAUCUAUCUAG
>mARM1801 (SEQ ID NO: 75)
AUUAUUACAUCAAAACAAAAAGCCGCCAAUGCUGUUCAACCUGCGCAUCCUGCU
GAACAACGCCGCCUUCCGCAACGGCCACAACUUCAUGGUGCGCAACUUCCGCUGC
GGCCAGCCCCUGCAGAACAAGGUGCAGCUGAAGGGCCGCGACCUGCUGACCCUGA
AGAACUUCACCGGCGAGGAGAUCAAGUACAUGCUGUGGCUGAGCGCCGACCUGA
AGUUCCGCAUCAAGCAGAAGGGCGAGUACCUGCCCCUGCUGCAGGGCAAGAGCCU
GGGCAUGAUCUUCGAGAAGCGCAGCACCCGCACCCGCCUGAGCACCGAGACAGGC
UUCGCCCUGCUGGGCGGCCACCCCUGCUUCCUGACCACCCAGGACAUCCACCUGG
GCGUGAACGAGAGCCUGACCGACACCGCCCGCGUGCUGAGCAGCAUGGCCGACGC
CGUGCUGGCCCGCGUGUACAAGCAGAGCGACCUGGACACCCUGGCCAAGGAGGCC
AGCAUCCCCAUCAUCAACGGCCUGAGCGACCUGUACCACCCCAUCCAGAUCCUGG
CCGACUACCUGACCCUGCAGGAGCACUACAGCAGCCUGAAGGGCCUGACCCUGAG
CUGGAUCGGCGACGGCAACAACAUCCUGCACAGCAUCAUGAUGAGCGCCGCCAAG
UUCGGCAUGCACCUGCAGGCCGCCACCCCCAAGGGCUACGAGCCCGACGCCAGCG
UGACCAAGCUGGCCGAGCAGUACGCCAAGGAGAACGGCACCAAGCUGCUGCUGAC
CAACGACCCCCUGGAGGCCGCCCACGGCGGCAACGUGCUGAUCACCGACACCUGG
AUCAGCAUGGGCCAGGAGGAGGAGAAGAAGAAGCGCCUGCAGGCCUUCCAGGGC
UACCAGGUGACCAUGAAGACCGCCAAGGUGGCCGCCAGCGACUGGACCUUCCUGC
ACUGCCUGCCCCGCAAGCCCGAGGAGGUGGACGACGAGGUGUUCUACAGCCCCCG
CAGCCUGGUGUUCCCCGAGGCCGAGAACCGCAAGUGGACCAUCAUGGCCGUGAUG
GUGAGCCUGCUGACCGACUACAGCCCCCAGCUGCAGAAGCCCAAGUUCUGAACGC
CGAAGCCUGCAGCCAUGCGACCCCACGCCACCCCGUGCCUCCUGCCUCCGCGCAG
CCUGCAGCGGGAGACCCUGUCCCCGCCCCAGCCGUCCUCCUGGGGUGGACCCUAG
UUUAAUAAAGAUUCACCAAGUUUCACGC
>mARM1802 (SEQ ID NO: 76)
CUUAAGGGGGCGCUGCCUACGGAGGUGGCAGCCAUCUCCUUCUCGGCAUCAAGCU
UACCAUGGUGCCCCAGGCCCUGCUCUUGGUCCCGCUGCUGGUGUUCCCCCUCUGC
UUCGGCAAGUUCCCCAUCUACACCAUCCCCGACAAGCUGGGGCCGUGGAGCCCCA
UCGACAUCCACCACCUGUCCUGCCCCAACAACCUCGUGGUCGAGGACGAGGGCUG
CACCAACCUGAGCGGGUUCUCCUACAUGCUGUUCAACCUGCGCAUCCUGCUGAAC
AACGCCGCCUUCCGCAACGGCCACAACUUCAUGGUGCGCAACUUCCGCUGCGGCC
AGCCCCUGCAGAACAAGGUGCAGCUGAAGGGCCGCGACCUGCUGACCCUGAAGAA
CUUCACCGGCGAGGAGAUCAAGUACAUGCUGUGGCUGAGCGCCGACCUGAAGUU
CCGCAUCAAGCAGAAGGGCGAGUACCUGCCCCUGCUGCAGGGCAAGAGCCUGGGC
AUGAUCUUCGAGAAGCGCAGCACCCGCACCCGCCUGAGCACCGAGACAGGCUUCG
CCCUGCUGGGCGGCCACCCCUGCUUCCUGACCACCCAGGACAUCCACCUGGGCGU
GAACGAGAGCCUGACCGACACCGCCCGCGUGCUGAGCAGCAUGGCCGACGCCGUG
CUGGCCCGCGUGUACAAGCAGAGCGACCUGGACACCCUGGCCAAGGAGGCCAGCA
UCCCCAUCAUCAACGGCCUGAGCGACCUGUACCACCCCAUCCAGAUCCUGGCCGA
CUACCUGACCCUGCAGGAGCACUACAGCAGCCUGAAGGGCCUGACCCUGAGCUGG
AUCGGCGACGGCAACAACAUCCUGCACAGCAUCAUGAUGAGCGCCGCCAAGUUCG
GCAUGCACCUGCAGGCCGCCACCCCCAAGGGCUACGAGCCCGACGCCAGCGUGAC
CAAGCUGGCCGAGCAGUACGCCAAGGAGAACGGCACCAAGCUGCUGCUGACCAAC
GACCCCCUGGAGGCCGCCCACGGCGGCAACGUGCUGAUCACCGACACCUGGAUCA
GCAUGGGCCAGGAGGAGGAGAAGAAGAAGCGCCUGCAGGCCUUCCAGGGCUACC
AGGUGACCAUGAAGACCGCCAAGGUGGCCGCCAGCGACUGGACCUUCCUGCACUG
CCUGCCCCGCAAGCCCGAGGAGGUGGACGACGAGGUGUUCUACAGCCCCCGCAGC
CUGGUGUUCCCCGAGGCCGAGAACCGCAAGUGGACCAUCAUGGCCGUGAUGGUG
AGCCUGCUGACCGACUACAGCCCCCAGCUGCAGAAGCCCAAGUUCUGAAUAAGUG
AAUGCAAGGCUGGCCGGAAGCCCUUGCCUGAAAGCAAGAUUUCAGCCUGGAAGA
GGGCAAAGUGGACGGGAGUGGACAGGAGUGGAUGCGAUAAGAUGUGGUUUGAAG
CUGAUGGGUGCCAGCCCUGCAUUGCUGAGUCAAUCAAUAAAGAGCUUUCUUUUG
ACCCAUUCUAGAUCUAG
>mARM1803 (SEQ ID NO: 77)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGGGCGUCUUCAACCUGCGGAUCCUGCU
GAACAACGCCGCCUUCCGGAACGGCCACAACUUCAUGGUCCGCAACUUCAGAUGC
GGCCAGCCCCUGCAGAACAAGGUGCAGCUGAAGGGCCGGGACCUGCUGACCCUGA
AGAACUUCACCGGCGAAGAGAUCAAGUACAUGCUGUGGCUGAGCGCCGACCUGA
AGUUCCGGAUCAAGCAGAAGGGCGAGUACCUGCCCCUGCUGCAAGGCAAGAGCC
UGGGCAUGAUCUUCGAGAAGCGGAGCACCCGGACCCGGCUGAGCACCGAGACAG
GCUUUGCCCUGCUGGGAGGCCACCCCUGCUUUCUGACCACCCAGGACAUCCACCU
GGGCGUGAACGAGAGCCUGACCGACACCGCCAGAGUGCUGAGCAGCAUGGCCGAC
GCCGUGCUGGCCCGGGUGUACAAGCAGAGCGACCUGGACACCCUGGCCAAAGAGG
CCAGCAUCCCCAUCAUCAACGGCCUGAGCGACCUGUACCACCCCAUCCAGAUCCU
GGCCGACUACCUGACCCUGCAGGAACACUACAGCUCCCUGAAGGGCCUGACCCUG
AGCUGGAUCGGCGACGGCAACAACAUCCUGCACAGCAUCAUGAUGAGCGCCGCCA
AGUUCGGCAUGCAUCUGCAGGCCGCCACCCCCAAGGGCUACGAGCCUGAUGCCAG
CGUGACCAAGCUGGCCGAGCAGUACGCCAAAGAGAACGGCACCAAGCUGCUGCUG
ACCAACGACCCCCUGGAAGCCGCCCACGGCGGCAACGUGCUGAUCACCGACACCU
GGAUCAGCAUGGGCCAGGAAGAGGAAAAGAAGAAGCGGCUGCAGGCCUUCCAGG
GCUACCAGGUCACAAUGAAGACCGCCAAGGUGGCCGCCAGCGACUGGACCUUCCU
GCACUGCCUGCCCCGGAAGCCCGAAGAGGUGGACGACGAGGUGUUCUACAGCCCC
CGGUCCCUGGUGUUCCCCGAGGCCGAGAACCGGAAGUGGACCAUUAUGGCCGUG
AUGGUGUCCCUGCUGACCGACUACUCCCCCCAGCUGCAGAAGCCCAAGUUCUAGA
UAAGUGAACUCGAGCUAGUGACUGACUAGGAUCUGGUUACCACUAAACCAGCCU
CAAGAACACCCGAAUGGAGUCUCUAAGCUACAUAAUACCAACUUACACUUACAA
AAUGUUGUCCCCCAAAAUGUAGCCAUUCGUAUCUGCUCCUAAUAAAAAGAAAGU
UUCUUCACAUUCUAG
>mARM1804 (SEQ ID NO: 78)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGGGCGUCUUCAACCUGCGGAUCCUGCU
GAACAACGCCGCCUUCCGGAACGGCCACAACUUCAUGGUCCGCAACUUCAGAUGC
GGCCAGCCCCUGCAGAACAGGGUGCAGCUGAAGGGCCGGGACCUGCUGACCCUGA
AGAACUUCACCGGCGAAGAGAUCAGGUACAUGCUGUGGCUGAGCGCCGACCUGA
AGUUCCGGAUCAAGCAGAAGGGCGAGUACCUGCCCCUGCUGCAAGGCAAGAGCC
UGGGCAUGAUCUUCGAGAAGCGGAGCACCCGGACCCGGCUGAGCACCGAGACAG
GCUUUGCCCUGCUGGGAGGCCACCCCUGCUUUCUGACCACCCAGGACAUCCACCU
GGGCGUGAACGAGAGCCUGACCGACACCGCCAGAGUGCUGAGCAGCAUGGCCGAC
GCCGUGCUGGCCCGGGUGUACAAGCAGAGCGACCUGGACACCCUGGCCAAAGAGG
CCAGCAUCCCCAUCAUCAACGGCCUGAGCGACCUGUACCACCCCAUCCAGAUCCU
GGCCGACUACCUGACCCUGCAGGAACACUACAGCUCCCUGAAGGGCCUGACCCUG
AGCUGGAUCGGCGACGGCAACAACAUCCUGCACAGCAUCAUGAUGAGCGCCGCCA
AGUUCGGCAUGCAUCUGCAGGCCGCCACCCCCAAGGGCUACGAGCCUGAUGCCAG
CGUGACCAAGCUGGCCGAGCAGUACGCCAAAGAGAACGGCACCAAGCUGCUGCUG
ACCAACGACCCCCUGGAAGCCGCCCACGGCGGCAACGUGCUGAUCACCGACACCU
GGAUCAGCAUGGGCCAGGAAGAGGAAAAGAAGAAGCGGCUGCAGGCCUUCCAGG
GCUACCAGGUCACAAUGAAGACCGCCAAGGUGGCCGCCAGCGACUGGACCUUCCU
GCACUGCCUGCCCCGGAAGCCCGAAGAGGUGGACGACGAGGUGUUCUACAGCCCC
CGGUCCCUGGUGUUCCCCGAGGCCGAGAACCGGAAGUGGACCAUUAUGGCCGUG
AUGGUGUCCCUGCUGACCGACUACUCCCCCCAGCUGCAGAAGCCCAAGUUCUAGA
UAAGUGAACUCGAGCUAGUGACUGACUAGGAUCUGGUUACCACUAAACCAGCCU
CAAGAACACCCGAAUGGAGUCUCUAAGCUACAUAAUACCAACUUACACUUACAA
AAUGUUGUCCCCCAAAAUGUAGCCAUUCGUAUCUGCUCCUAAUAAAAAGAAAGU
UUCUUCACAUUCUAG
>mARM1805 (SEQ ID NO: 79)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUGGUCUUCAACCUGCGGAUCCUGCU
GAACAACGCCGCCUUCCGGAACGGCCACAACUUCAUGGUCCGCAACUUCAGAUGC
GGCCAGCCCCUGCAGAACAGGGUGCAGCUGAAGGGCCGGGACCUGCUGACCCUGA
AGAACUUCACCGGCGAAGAGAUCAGGUACAUGCUGUGGCUGAGCGCCGACCUGA
AGUUCCGGAUCAAGCAGAAGGGCGAGUACCUGCCCCUGCUGCAAGGCAAGAGCC
UGGGCAUGAUCUUCGAGAAGCGGAGCACCCGGACCCGGCUGAGCACCGAGACAG
GCUUUGCCCUGCUGGGAGGCCACCCCUGCUUUCUGACCACCCAGGACAUCCACCU
GGGCGUGAACGAGAGCCUGACCGACACCGCCAGAGUGCUGAGCAGCAUGGCCGAC
GCCGUGCUGGCCCGGGUGUACAAGCAGAGCGACCUGGACACCCUGGCCAAAGAGG
CCAGCAUCCCCAUCAUCAACGGCCUGAGCGACCUGUACCACCCCAUCCAGAUCCU
GGCCGACUACCUGACCCUGCAGGAACACUACAGCUCCCUGAAGGGCCUGACCCUG
AGCUGGAUCGGCGACGGCAACAACAUCCUGCACAGCAUCAUGAUGAGCGCCGCCA
AGUUCGGCAUGCAUCUGCAGGCCGCCACCCCCAAGGGCUACGAGCCUGAUGCCAG
CGUGACCAAGCUGGCCGAGCAGUACGCCAAAGAGAACGGCACCAAGCUGCUGCUG
ACCAACGACCCCCUGGAAGCCGCCCACGGCGGCAACGUGCUGAUCACCGACACCU
GGAUCAGCAUGGGCCAGGAAGAGGAAAAGAAGAAGCGGCUGCAGGCCUUCCAGG
GCUACCAGGUCACAAUGAAGACCGCCAAGGUGGCCGCCAGCGACUGGACCUUCCU
GCACUGCCUGCCCCGGAAGCCCGAAGAGGUGGACGACGAGGUGUUCUACAGCCCC
CGGUCCCUGGUGUUCCCCGAGGCCGAGAACCGGAAGUGGACCAUUAUGGCCGUG
AUGGUGUCCCUGCUGACCGACUACUCCCCCCAGCUGCAGAAGCCCAAGUUCUAGA
UAAGUGAACUCGAGCUAGUGACUGACUAGGAUCUGGUUACCACUAAACCAGCCU
CAAGAACACCCGAAUGGAGUCUCUAAGCUACAUAAUACCAACUUACACUUACAA
AAUGUUGUCCCCCAAAAUGUAGCCAUUCGUAUCUGCUCCUAAUAAAAAGAAAGU
UUCUUCACAUUCUAG
>mARM1806 (SEQ ID NO: 80)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUGUUCAACCUGAGGAUCCUGCUGA
ACAACGCAGCUUUCAGGAACGGCCACAACUUCAUGGUGAGGAACUUCCGGUGCG
GCCAGCCCCUGCAGAACAAGGUGCAGCUGAAGGGCAGGGACCUGCUGACCCUGAA
GAACUUCACCGGAGAGGAGAUCAAGUACAUGCUGUGGCUGAGCGCAGACCUGAA
GUUCAGGAUCAAGCAGAAGGGAGAGUACCUGCCCCUGCUGCAGGGGAAGUCCCU
GGGCAUGAUCUUCGAGAAGAGGAGUACCAGGACCAGGCUGAGCACCGAAACCGG
CUUCGCCCUGCUGGGAGGACACCCCUGCUUCCUGACCACCCAGGACAUCCACCUG
GGCGUGAACGAGAGUCUGACCGACACCGCCAGGGUGCUGUCUAGCAUGGCCGAC
GCCGUGCUGGCCAGGGUGUACAAGCAGUCAGACCUGGACACCCUGGCUAAGGAG
GCCAGCAUCCCCAUCAUCAACGGCCUGAGCGACCUGUACCACCCCAUCCAGAUCC
UGGCUGACUACCUGACCCUGCAGGAGCACUACAGCUCUCUGAAGGGCCUGACCCU
GAGCUGGAUCGGCGACGGGAACAACAUCCUGCACAGCAUCAUGAUGAGCGCCGCC
AAGUUCGGCAUGCACCUGCAGGCCGCUACCCCCAAGGGUUACGAGCCCGACGCCA
GCGUGACCAAGCUGGCAGAGCAGUACGCCAAGGAGAACGGCACCAAGCUGCUGC
UGACCAACGACCCCCUGGAGGCCGCCCACGGAGGCAACGUGCUGAUCACCGACAC
CUGGAUCAGCAUGGGACAGGAGGAGGAGAAGAAGAAGCGGCUGCAGGCUUUCCA
GGGUUACCAGGUGACCAUGAAGACCGCCAAGGUGGCUGCCAGCGACUGGACCUU
CCUGCACUGCCUGCCCAGGAAGCCCGAGGAGGUGGACGACGAGGUGUUCUACUCU
CCCAGGAGCCUGGUGUUCCCCGAGGCCGAGAACAGGAAGUGGACCAUCAUGGCU
GUGAUGGUGUCCCUGCUGACCGACUACAGCCCCCAGCUGCAGAAGCCCAAGUUCU
GAAUAAGUGAACUCGAGCUAGUGACUGACUAGGAUCUGGUUACCACUAAACCAG
CCUCAAGAACACCCGAAUGGAGUCUCUAAGCUACAUAAUACCAACUUACACUUAC
AAAAUGUUGUCCCCCAAAAUGUAGCCAUUCGUAUCUGCUCCUAAUAAAAAGAAA
GUUUCUUCACAUUCUAG
>mARM1808 (SEQ ID NO: 81)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUGUUCAACCUGAGGAUCCUGCUGA
ACAACGCAGCUUUCAGGAACGGCCACAACUUCAUGGUGAGGAACUUCCGGUGCG
GCCAGCCCCUGCAGAACAAGGUGCAGCUGAAGGGCAGGGACCUGCUGACCCUGAA
GAACUUCACCGGAGAGGAGAUCAAGUACAUGCUGUGGCUGAGCGCAGACCUGAA
GUUCAGGAUCAAGCAGAAGGGAGAGUACCUGCCCCUGCUGCAGGGGAAGUCCCU
GGGCAUGAUCUUCGAGAAGAGGAGUACCAGGACCAGGCUGAGCACCGAAACCGG
CUUCGCCCUGCUGGGAGGACACCCCUGCUUCCUGACGACCCAGGACAUCCACCUC
GGAGUGAACGAAUCCCUCACCGAUACCGCCCGGGUGUUAUCAAGCAUGGCAGAU
GCCGUGCUGGCCAGGGUGUACAAACAGUCCGAUCUGGACACUCUGGCUAAGGAG
GCCAGCAUCCCCAUCAUCAACGGCCUGAGCGACCUGUACCACCCCAUCCAGAUCC
UGGCUGACUACCUGACCCUGCAGGAGCACUACAGCUCUCUGAAGGGCCUGACCCU
GAGCUGGAUCGGCGACGGGAACAACAUCCUGCACUCCAUCAUGAUGUCCGCCGCG
AAGUUCGGAAUGCAUCUGCAAGCCGCCACGCCAAAAGGAUACGAACCGGAUGCG
CCCGUGACAAAGUUGGCGGAACAGUACGCUAAGGAGAACGGAACCAAGCUGCUG
CUGACCAACGACCCCCUGGAGGCCGCCCACGGAGGCAACGUGCUGAUCACCGACA
CCUGGAUCAGCAUGGGACAGGAGGAGGAGAAGAAGAAGCGGCUGCAGGCUUUCC
AGGGUUACCAGGUGACCAUGAAGACCGCCAAGGUGGCUGCCAGCGACUGGACCU
UCCUGCACUGCCUGCCCAGGAAGCCCGAGGAGGUGGACGACGAGGUGUUCUACUC
UCCCAGGAGCCUGGUGUUCCCCGAGGCCGAGAACAGGAAGUGGACCAUCAUGGC
UGUGAUGGUGUCCCUGCUGACCGACUACAGCCCCCAGCUGCAGAAGCCCAAGUUC
UGAAUAAGUGAACUCGAGCUAGUGACUGACUAGGAUCUGGUUACCACUAAACCA
GCCUCAAGAACACCCGAAUGGAGUCUCUAAGCUACAUAAUACCAACUUACACUU
ACAAAAUGUUGUCCCCCAAAAUGUAGCCAUUCGUAUCUGCUCCUAAUAAAAAGA
AAGUUUCUUCACAUUCUAG
>mARM1809 (SEQ ID NO: 82)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUGUUCAACCUGCGCAUCCUGCUGAA
CAACGCCGCCUUCCGCAACGGCCACAACUUCAUGGUGCGCAACUUCCGCUGCGGC
CAGCCCCUGCAGAACAAGGUGCAGCUGAAGGGCCGCGACCUGCUGACCCUGAAGA
ACUUCACCGGCGAGGAGAUCAAGUACAUGCUGUGGCUGAGCGCCGACCUGAAGU
UCCGCAUCAAGCAGAAGGGCGAGUACCUGCCCCUGCUGCAGGGCAAGAGCCUGGG
CAUGAUCUUCGAGAAGCGCAGCACCCGCACCCGCCUGAGCACCGAGACAGGCUUC
GCCCUGCUGGGCGGCCACCCCUGCUUCCUGACCACCCAGGACAUCCACCUGGGCG
UGAACGAGAGCCUGACCGACACCGCCCGCGUGCUGAGCAGCAUGGCCGACGCCGU
GCUGGCCCGCGUGUACAAGCAGAGCGACCUGGACACCCUGGCCAAGGAGGCCAGC
AUCCCCAUCAUCAACGGCCUGAGCGACCUGUACCACCCCAUCCAGAUCCUGGCCG
ACUACCUGACCCUGCAGGAGCACUACAGCAGCCUGAAGGGCCUGACCCUGAGCUG
GAUCGGCGACGGCAACAACAUCCUGCACAGCAUCAUGAUGAGCGCCGCCAAGUUC
GGCAUGCACCUGCAGGCCGCCACCCCCAAGGGCUACGAGCCCGACGCCAGCGUGA
CCAAGCUGGCCGAGCAGUACGCCAAGGAGAACGGCACCAAGCUGCUGCUGACCAA
CGACCCCCUGGAGGCCGCCCACGGCGGCAACGUGCUGAUCACCGACACCUGGAUC
AGCAUGGGCCAGGAGGAGGAGAAGAAGAAGCGCCUGCAGGCCUUCCAGGGCUAC
CAGGUGACCAUGAAGACCGCCAAGGUGGCCGCCAGCGACUGGACCUUCCUGCACU
GCCUGCCCCGCAAGCCCGAGGAGGUGGACGACGAGGUGUUCUACAGCCCCCGCAG
CCUGGUGUUCCCCGAGGCCGAGAACCGCAAGUGGACCAUCAUGGCCGUGAUGGU
GAGCCUGCUGACCGACUACAGCCCCCAGCUGCAGAAGCCCAAGUUCUGAAUAAGU
GAACUCGAGCUAGUGACUGACUAGGAUCUGGUUACCACUAAACCAGCCUCAAGA
ACACCCGAAUGGAGUCUCUAAGCUACAUAAUACCAACUUACACUUACAAAAUGU
UGUCCCCCAAAAUGUAGCCAUUCGUAUCUGCUCCUAAUAAAAAGAAAGUUUCUU
CACAUUCUAG
>mARM1816 (SEQ ID NO: 83)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCAUGCUGUUCAACCUGCGCAUCCUGCUGAACAA
CGCCGCCUUCCGCAACGGCCACAACUUCAUGGUGCGCAACUUCCGCUGCGGCCAG
CCCCUGCAGAACAAGGUGCAGCUGAAGGGCCGCGACCUGCUGACCCUGAAGAACU
UCACCGGCGAGGAGAUCAAGUACAUGCUGUGGCUGAGCGCCGACCUGAAGUUCC
GCAUCAAGCAGAAGGGCGAGUACCUGCCCCUGCUGCAGGGCAAGAGCCUGGGCA
UGAUCUUCGAGAAGCGCAGCACCCGCACCCGCCUGAGCACCGAGACAGGCUUCGC
CCUGCUGGGCGGCCACCCCUGCUUCCUGACCACCCAGGACAUCCACCUGGGCGUG
AACGAGAGCCUGACCGACACCGCCCGCGUGCUGAGCAGCAUGGCCGACGCCGUGC
UGGCCCGCGUGUACAAGCAGAGCGACCUGGACACCCUGGCCAAGGAGGCCAGCAU
CCCCAUCAUCAACGGCCUGAGCGACCUGUACCACCCCAUCCAGAUCCUGGCCGAC
UACCUGACCCUGCAGGAGCACUACAGCAGCCUGAAGGGCCUGACCCUGAGCUGGA
UCGGCGACGGCAACAACAUCCUGCACAGCAUCAUGAUGAGCGCCGCCAAGUUCGG
CAUGCACCUGCAGGCCGCCACCCCCAAGGGCUACGAGCCCGACGCCAGCGUGACC
AAGCUGGCCGAGCAGUACGCCAAGGAGAACGGCACCAAGCUGCUGCUGACCAACG
ACCCCCUGGAGGCCGCCCACGGCGGCAACGUGCUGAUCACCGACACCUGGAUCAG
CAUGGGCCAGGAGGAGGAGAAGAAGAAGCGCCUGCAGGCCUUCCAGGGCUACCA
GGUGACCAUGAAGACCGCCAAGGUGGCCGCCAGCGACUGGACCUUCCUGCACUGC
CUGCCCCGCAAGCCCGAGGAGGUGGACGACGAGGUGUUCUACAGCCCCCGCAGCC
UGGUGUUCCCCGAGGCCGAGAACCGCAAGUGGACCAUCAUGGCCGUGAUGGUGA
GCCUGCUGACCGACUACAGCCCCCAGCUGCAGAAGCCCAAGUUCUGACUAGUGAC
UGACUAGGAUCUGGUUACCACUAAACCAGCCUCAAGAACACCCGAAUGGAGUCU
CUAAGCUACAUAAUACCAACUUACACUUACAAAAUGUUGUCCCCCAAAAUGUAG
CCAUUCGUAUCUGCUCCUAAUAAAAAGAAAGUUUCUUCACAUUCUAG
>mARM1822 (SEQ ID NO: 84)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAACUUGAGAAUCCUGCUGA
ACAACGCCGCCUUUCGCAACGGUCACAAUUUUAUGGUCAGAAACUUCAGAUGCG
GACAGCCCCUCCAAAACAAGGUCCAGCUGAAGGGCCGCGAUCUCCUCACCCUGAA
GAACUUCACGGGGGAGGAGAUCAAGUACAUGCUGUGGCUCUCCGCUGACCUGAA
GUUCAGGAUCAAGCAGAAGGGAGAAUAUCUGCCGCUGCUGCAAGGGAAGUCCCU
GGGGAUGAUUUUCGAGAAGCGGAGCACCCGGACUCGGCUCUCCACUGAAACUGG
UUUCGCCCUUCUGGGCGGUCACCCCUGCUUCCUGACCACUCAAGACAUUCACCUC
GGAGUGAACGAGUCCUUGACUGACACCGCCCGGGUGCUGUCGAGCAUGGCAGAC
GCCGUGCUAGCCCGCGUGUACAAGCAGUCAGACCUCGAUACCCUGGCCAAGGAGG
CUUCGAUCCCGAUCAUCAACGGGUUGUCCGACCUGUACCACCCGAUUCAGAUUCU
CGCCGACUACCUCACCCUGCAAGAGCAUUACAGCUCCCUGAAGGGGCUUACCCUG
UCCUGGAUUGGCGACGGAAACAACAUCCUGCACUCCAUUAUGAUGUCGGCGGCC
AAGUUCGGCAUGCACCUCCAAGCCGCGACCCCUAAGGGUUACGAACCAGACGCGU
CAGUGACUAAGCUGGCCGAACAGUACGCAAAGGAAAAUGGCACGAAGCUGCUCC
UGACCAACGAUCCGUUGGAAGCCGCCCAUGGCGGAAAUGUGCUCAUCACCGACAC
CUGGAUCUCGAUGGGACAGGAGGAAGAGAAGAAGAAGCGGCUGCAGGCGUUCCA
GGGCUACCAGGUCACCAUGAAAACUGCCAAGGUGGCCGCCAGCGACUGGACCUUC
CUGCACUGCCUUCCGCGCAAGCCUGAGGAGGUGGACGAUGAAGUGUUCUACUCU
CCACGGUCCCUGGUGUUCCCCGAGGCGGAGAACCGCAAAUGGACCAUCAUGGCUG
UGAUGGUCAGCCUGCUGACCGAUUACAGCCCUCAGUUGCAAAAGCCGAAGUUUU
GAUAACUCGAGCUAGUGACUGACUAGGAUCUGGUUACCACUAAACCAGCCUCAA
GAACACCCGAAUGGAGUCUCUAAGCUACAUAAUACCAACUUACACUUACAAAAU
GUUGUCCCCCAAAAUGUAGCCAUUCGUAUCUGCUCCUAAUAAAAAGAAAGUUUC
UUCACAUUCUAG
>mARM1823 (SEQ ID NO: 85)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUGUUCAACCUCCGCAUCCUCCUCAA
CAACGCCGCAUUCAGAAACGGGCACAACUUCAUGGUCAGAAACUUCCGCUGCGGG
CAACCCCUACAAAACAAGGUCCAGCUCAAGGGGCGGGACCUCCUGACCCUGAAGA
ACUUCACCGGCGAAGAGAUCAAGUACAUGCUGUGGCUCUCCGCCGACCUGAAGU
UCCGCAUCAAGCAGAAGGGAGAGUACCUCCCGCUGCUGCAAGGGAAGUCGCUGG
GGAUGAUCUUCGAGAAGCGGUCAACCAGAACCCGGCUGUCAACCGAAACCGGGU
UCGCACUGCUGGGGGGACACCCGUGCUUCCUGACCACCCAAGACAUCCACCUGGG
AGUGAACGAAUCGCUGACCGACACCGCCCGCGUGCUGAGCUCAAUGGCGGACGCC
GUGCUGGCCCGCGUGUACAAGCAGUCCGACCUGGACACCCUGGCCAAGGAAGCGU
CCAUCCCGAUCAUCAACGGACUGUCCGACCUGUACCACCCGAUCCAGAUCCUGGC
AGACUACCUGACCCUGCAAGAACACUACAGCUCCCUGAAGGGCCUGACCCUGUCA
UGGAUCGGGGACGGGAACAACAUCCUGCACUCCAUAAUGAUGUCAGCCGCCAAG
UUCGGAAUGCACCUCCAAGCCGCAACCCCGAAGGGCUACGAACCGGACGCAUCAG
UGACCAAACUGGCCGAGCAGUACGCCAAGGAAAACGGCACCAAGCUCCUGCUGAC
CAACGACCCGCUGGAGGCCGCACACGGGGGGAACGUGCUGAUCACCGACACCUGG
AUCUCCAUGGGACAGGAGGAGGAAAAGAAGAAGCGGCUGCAGGCGUUCCAGGGG
UACCAGGUCACCAUGAAAACCGCGAAGGUCGCGGCAUCAGACUGGACCUUCCUGC
ACUGCCUGCCCCGGAAGCCGGAAGAGGUGGACGACGAGGUGUUCUACUCGCCGCG
CUCGCUGGUGUUCCCCGAGGCGGAGAACAGGAAGUGGACCAUCAUGGCGGUGAU
GGUCAGCCUCCUGACCGACUACUCGCCGCAGCUGCAGAAGCCGAAGUUCUGAUAA
CUCGAGCUAGUGACUGACUAGGAUCUGGUUACCACUAAACCAGCCUCAAGAACA
CCCGAAUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUCUUCAC
AUUCUAG
>mARM1840 (SEQ ID NO: 86)
CUCCCUCCCCCCCCCCUAACGUUACUGGCCGAAGCCGCUUGGAAUAAGGCCGGUG
UGCGUUUGUCUAUAUGUUAUUUUCCACCAUAUUGCCGUCUUUUGGCAAUGUGAG
GGCCCGGAAACCUGGCCCUGUCUUCUUGACGAGCAUUCCUAGGGGUCUUUCCCCU
CUCGCCAAAGGAAUGCAAGGUCUGUUGAAUGUCGUGAAGGAAGCAGUUCCUCUG
GAAGCUUCUUGAAGACAAACAACGUCUGUAGCGACCCUUUGCAGGCAGCGGAAC
CCCCCACCUGGCGACAGGUGCCUCUGCGGCCAAAAGCCACGUGUAUAAGAUACAC
CUGCAAAGGCGGCACAACCCCAGUGCCACGUUGUGAGUUGGAUAGUUGUGGAAA
GAGUCAAAUGGCUCUCCUCAAGCGUAUUCAACAAGGGGCUGAAGGAUGCCCAGA
AGGUACCCCAUUGUAUGGGAUCUGAUCUGGGGCCUCGGUGCACAUGCUUUACGU
GUGUUUAGUCGAGGUUAAAAAACGUCUAGGCCCCCCGAACCACGGGGACGUGGU
UUUCCUUUGAAAAACACGAUGAUAAUAUGCUUUUCAAUCUCCGCAUCCUCCUUA
ACAACGCCGCGUUUAGAAACGGCCACAACUUCAUGGUCCGGAACUUCAGAUGUG
GCCAGCCGCUUCAAAACAAGGUCCAGCUGAAGGGCCGGGAUCUUCUGACCCUGAA
GAACUUUACUGGCGAAGAGAUCAAGUACAUGCUCUGGCUCUCCGCGGACUUGAA
GUUCCGCAUUAAGCAGAAGGGGGAAUACCUUCCGCUGCUUCAAGGAAAGAGCCU
CGGCAUGAUCUUUGAGAAGCGCUCAACCAGGACCCGCCUUUCUACUGAAACUGG
GUUCGCGCUGCUCGGUGGCCACCCCUGCUUCCUGACGACCCAGGACAUCCACCUC
GGAGUGAACGAAUCCCUCACCGAUACCGCCCGGGUGUUAUCGAGCAUGGCAGAU
GCCGUGCUGGCCAGGGUGUACAAACAGUCCGAUCUGGACACUCUGGCCAAGGAG
GCGUCAAUUCCUAUUAUCAACGGCCUUAGUGACCUCUACCAUCCGAUUCAGAUCC
UGGCCGAUUACCUCACCCUGCAAGAACACUACAGCUCCCUGAAGGGUCUGACAUU
GUCCUGGAUCGGCGACGGCAACAACAUUCUCCAUUCCAUCAUGAUGUCCGCCGCA
AAAUUCGGCAUGCAUCUUCAAGCCGCCACGCCGAAGGGUUACGAGCCCGACGCUU
CCGUGACUAAGCUCGCCGAGCAGUACGCUAAGGAGAACGGAACCAAGCUUCUGC
UGACUAACGACCCACUAGAAGCAGCCCACGGGGGCAACGUGCUUAUUACUGACAC
CUGGAUCUCCAUGGGCCAGGAAGAAGAGAAAAAGAAGCGGCUGCAGGCGUUCCA
GGGAUAUCAGGUCACCAUGAAAACCGCCAAGGUCGCUGCCUCCGACUGGACCUUC
CUGCACUGCCUGCCUCGCAAGCCUGAAGAAGUGGACGACGAGGUGUUCUACUCGC
CACGGAGCCUCGUGUUCCCCGAGGCCGAGAAUAGAAAGUGGACCAUCAUGGCCG
UGAUGGUGUCACUGCUCACCGACUACAGCCCGCAGCUUCAGAAGCCCAAGUUCUG
AAUAAGUAGAUAGUGCAGUCACUGGCACAACGCGUUGCCCGGUAAGCCAAUCGG
GUAUACACGGUCGUCAUACUGCAGACAGGGUUCUUCUACUUUGCAAGAUAGUCU
AGAGUAGUAAAAUAAAUAGUAUAAGUCUAG
>mARM1841 (SEQ ID NO: 87)
CUCCCUCCCCCCCCCCUAACGUUACUGGCCGAAGCCGCUUGGAAUAAGGCCGGUG
UGCGUUUGUCUAUAUGUUAUUUUCCACCAUAUUGCCGUCUUUUGGCAAUGUGAG
GGCCCGGAAACCUGGCCCUGUCUUCUUGACGAGCAUUCCUAGGGGUCUUUCCCCU
CUCGCCAAAGGAAUGCAAGGUCUGUUGAAUGUCGUGAAGGAAGCAGUUCCUCUG
GAAGCUUCUUGAAGACAAACAACGUCUGUAGCGACCCUUUGCAGGCAGCGGAAC
CCCCCACCUGGCGACAGGUGCCUCUGCGGCCAAAAGCCACGUGUAUAAGAUACAC
CUGCAAAGGCGGCACAACCCCAGUGCCACGUUGUGAGUUGGAUAGUUGUGGAAA
GAGUCAAAUGGCUCUCCUCAAGCGUAUUCAACAAGGGGCUGAAGGAUGCCCAGA
AGGUACCCCAUUGUAUGGGAUCUGAUCUGGGGCCUCGGUGCACAUGCUUUACGU
GUGUUUAGUCGAGGUUAAAAAACGUCUAGGCCCCCCGAACCACGGGGACGUGGU
UUUCCUUUGAAAAACACGAUGAUAAUAUGCUUUUCAACCUGAGAAUCCUCUUGA
ACAAUGCUGCUUUUCGGAAUGGCCACAACUUUAUGGUUCGGAACUUCCGUUGCG
GCCAGCCUUUACAAAACAAGGUCCAGCUGAAGGGCCGGGAUUUGCUCACACUAA
AGAACUUUACUGGAGAAGAGAUCAAGUACAUGCUAUGGCUGUCGGCCGACCUGA
AGUUCCGUAUCAAGCAGAAGGGAGAAUACCUUCCGCUGCUUCAAGGAAAGAGCC
UCGGCAUGAUCUUUGAGAAGCGCUCAACCAGGACCCGCCUUUCUACUGAAACUG
GGUUCGCGCUGCUCGGUGGCCACCCCUGCUUCCUGACGACCCAGGACAUCCACCU
CGGAGUGAACGAAUCCCUCACCGAUACCGCCCGGGUGUUAUCGAGCAUGGCAGA
UGCCGUGCUGGCCAGGGUGUACAAACAGUCCGAUCUCGAUACCUUGGCAAAGGA
GGCUUCCAUUCCCAUCAUCAACGGCCUGAGCGACCUGUACCACCCAAUCCAAAUC
CUGGCUGACUACCUGACCCUGCAAGAGCACUACAGCAGCCUGAAGGGUCUGACCC
UGUCAUGGAUUGGCGAUGGAAACAAUAUUCUGCACUCCAUCAUGAUGUCCGCCG
CGAAGUUCGGAAUGCAUCUGCAAGCCGCCACUCCAAAAGGAUACGAACCGGAUG
CAUCCGUGACCAAGUUGGCGGAACAGUACGCGAAGGAGAACGGAACCAAGCUCC
UGCUGACUAACGACCCGCUCGAGGCUGCGCAUGGGGGUAACGUGCUGAUUACGG
ACACCUGGAUCUCCAUGGGGCAGGAGGAAGAGAAGAAGAAGAGACUGCAGGCAU
UCCAGGGGUACCAGGUCACCAUGAAAACCGCAAAAGUGGCAGCUUCGGACUGGA
CUUUCCUGCAUUGCCUGCCGAGGAAGCCGGAGGAAGUCGACGACGAAGUGUUCU
ACUCGCCUCGGUCCCUGGUGUUCCCCGAGGCCGAAAACCGGAAGUGGACCAUCAU
GGCCGUGAUGGUGUCCUUGCUGACUGACUAUAGCCCGCAGCUGCAGAAGCCUAA
GUUCUGAAUAAGUAGAUAGUGCAGUCACUGGCACAACGCGUUGCCCGGUAAGCC
AAUCGGGUAUACACGGUCGUCAUACUGCAGACAGGGUUCUUCUACUUUGCAAGA
UAGUCUAGAGUAGUAAAAUAAAUAGUAUAAGUCUAG
>mARM1842 (SEQ ID NO: 88)
CUCCCUCCCCCCCCCCUAACGUUACUGGCCGAAGCCGCUUGGAAUAAGGCCGGUG
UGCGUUUGUCUAUAUGUUAUUUUCCACCAUAUUGCCGUCUUUUGGCAAUGUGAG
GGCCCGGAAACCUGGCCCUGUCUUCUUGACGAGCAUUCCUAGGGGUCUUUCCCCU
CUCGCCAAAGGAAUGCAAGGUCUGUUGAAUGUCGUGAAGGAAGCAGUUCCUCUG
GAAGCUUCUUGAAGACAAACAACGUCUGUAGCGACCCUUUGCAGGCAGCGGAAC
CCCCCACCUGGCGACAGGUGCCUCUGCGGCCAAAAGCCACGUGUAUAAGAUACAC
CUGCAAAGGCGGCACAACCCCAGUGCCACGUUGUGAGUUGGAUAGUUGUGGAAA
GAGUCAAAUGGCUCUCCUCAAGCGUAUUCAACAAGGGGCUGAAGGAUGCCCAGA
AGGUACCCCAUUGUAUGGGAUCUGAUCUGGGGCCUCGGUGCACAUGCUUUACGU
GUGUUUAGUCGAGGUUAAAAAACGUCUAGGCCCCCCGAACCACGGGGACGUGGU
UUUCCUUUGAAAAACACGAUGAUAAUAUGCUGUUCAACCUGCGCAUCCUGCUGA
ACAACGCCGCCUUCCGCAACGGCCACAACUUCAUGGUGCGCAACUUCCGCUGCGG
CCAGCCCCUGCAGAACAAGGUGCAGCUGAAGGGCCGCGACCUGCUGACCCUGAAG
AACUUCACCGGCGAGGAGAUCAAGUACAUGCUGUGGCUGAGCGCCGACCUGAAG
UUCCGCAUCAAGCAGAAGGGCGAGUACCUGCCCCUGCUGCAGGGCAAGAGCCUGG
GCAUGAUCUUCGAGAAGCGCAGCACCCGCACCCGCCUGAGCACCGAGACAGGCCU
GGCCCUGCUGGGCGGCCACCCCUGCUUCCUGACCACCCAGGACAUCCACCUGGGC
GUGAACGAGAGCCUGACCGACACCGCCCGCGUGCUGAGCAGCAUGGCCGACGCCG
UGCUGGCCCGCGUGUACAAGCAGAGCGACCUGGACACCCUGGCCAAGGAGGCCAG
CAUCCCCAUCAUCAACGGCCUGAGCGACCUGUACCACCCCAUCCAGAUCCUGGCC
GACUACCUGACCCUGCAGGAGCACUACAGCAGCCUGAAGGGCCUGACCCUGAGCU
GGAUCGGCGACGGCAACAACAUCCUGCACAGCAUCAUGAUGAGCGCCGCCAAGUU
CGGCAUGCACCUGCAGGCCGCCACCCCCAAGGGCUACGAGCCCGACGCCAGCGUG
ACCAAGCUGGCCGAGCAGUACGCCAAGGAGAACGGCACCAAGCUGCUGCUGACCA
ACGACCCCCUGGAGGCCGCCCACGGCGGCAACGUGCUGAUCACCGACACCUGGAU
CAGCAUGGGCCAGGAGGAGGAGAAGAAGAAGCGCCUGCAGGCCUUCCAGGGCUA
CCAGGUGACCAUGAAGACCGCCAAGGUGGCCGCCAGCGACUGGACCUUCCUGCAC
UGCCUGCCCCGCAAGCCCGAGGAGGUGGACGACGAGGUGUUCUACAGCCCCCGCA
GCCUGGUGUUCCCCGAGGCCGAGAACCGCAAGUGGACCAUCAUGGCCGUGAUGG
UGAGCCUGCUGACCGACUACAGCCCCCAGCUGCAGAAGCCCAAGUUCUGAAUAAG
UAGAUAGUGCAGUCACUGGCACAACGCGUUGCCCGGUAAGCCAAUCGGGUAUAC
ACGGUCGUCAUACUGCAGACAGGGUUCUUCUACUUUGCAAGAUAGUCUAGAGUA
GUAAAAUAAAUAGUAUAAGUCUAG
>mARM1843 (SEQ ID NO: 89)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAAUCUCCGCAUCCUCCUUAA
CAACGCCGCGUUUAGAAACGGCCACAACUUCAUGGUCCGGAACUUCAGAUGUGG
CCAGCCGCUUCAAAACAAGGUCCAGCUGAAGGGCCGGGAUCUUCUGACCCUGAAG
AACUUUACUGGCGAAGAGAUCAAGUACAUGCUCUGGCUCUCCGCGGACUUGAAG
UUCCGCAUUAAGCAGAAGGGGGAAUACCUUCCGCUGCUUCAAGGAAAGAGCCUC
GGCAUGAUCUUUGAGAAGCGCUCAACCAGGACCCGCCUUUCUACUGAAACUGGG
UUCGCGCUGCUCGGUGGCCACCCCUGCUUCCUGACGACCCAGGACAUCCACCUCG
GAGUGAACGAAUCCCUCACCGAUACCGCCCGGGUGUUAUCGAGCAUGGCAGAUG
CCGUGCUGGCCAGGGUGUACAAACAGUCCGAUCUGGACACUCUGGCCAAGGAGG
CGUCAAUUCCUAUUAUCAACGGCCUUAGUGACCUCUACCAUCCGAUUCAGAUCCU
GGCCGAUUACCUCACCCUGCAAGAACACUACAGCUCCCUGAAGGGUCUGACAUUG
UCCUGGAUCGGCGACGGCAACAACAUUCUCCAUUCCAUCAUGAUGUCCGCCGCAA
AAUUCGGCAUGCAUCUUCAAGCCGCCACGCCGAAGGGUUACGAGCCCGACGCUUC
CGUGACUAAGCUCGCCGAGCAGUACGCUAAGGAGAACGGAACCAAGCUUCUGCU
GACUAACGACCCACUAGAAGCAGCCCACGGGGGCAACGUGCUUAUUACUGACACC
UGGAUCUCCAUGGGCCAGGAAGAAGAGAAAAAGAAGCGGCUGCAGGCGUUCCAG
GGAUAUCAGGUCACCAUGAAAACCGCCAAGGUCGCUGCCUCCGACUGGACCUUCC
UGCACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCCGU
GAUGGUGUCACUGCUCACCGACUACAGCCCGCAGCUUCAGAAGCCCAAGUUCUAG
CUCGAGCUAGUGACUGACUAGGAUCUGGUUACCACUAAACCAGCCUCAAGAACA
CCCGAAUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUCUUCAC
AUUCUAG
>mARM1844 (SEQ ID NO: 90)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAAUCUCCGCAUCCUCCUUAA
CAACGCCGCGUUUAGAAACGGCCACAACUUCAUGGUCCGGAACUUCAGAUGUGG
CCAGCCGCUUCAAAACAAGGUCCAGCUGAAGGGCCGGGAUCUUCUGACCCUGAAG
AACUUUACUGGCGAAGAGAUCAAGUACAUGCUCUGGCUCUCCGCGGACUUGAAG
UUCCGCAUUAAGCAGAAGGGGGAAUACCUUCCGCUGCUUCAAGGAAAGAGCCUC
GGCAUGAUCUUUGAGAAGCGCUCAACCAGGACCCGCCUUUCUACUGAAACUGGG
UUCGCGCUGCUCGGUGGCCACCCCUGCUUCCUGACGACCCAGGACAUCCACCUCG
GAGUGAACGAAUCCCUCACCGAUACCGCCCGGGUGUUAUCGAGCAUGGCAGAUG
CCGUGCUGGCCAGGGUGUACAAACAGUCCGAUCUGGACACUCUGGCCAAGGAGG
CGUCAAUUCCUAUUAUCAACGGCCUUAGUGACCUCUACCAUCCGAUUCAGAUCCU
GGCCGAUUACCUCACCCUGCAAGAACACUACAGCUCCCUGAAGGGUCUGACAUUG
UCCUGGAUCGGCGACGGCAACAACAUUCUCCAUUCCAUCAUGAUGUCCGCCGCAA
AAUUCGGCAUGCAUCUUCAAGCCGCCACGCCGAAGGGUUACGAGCCCGACGCUUC
CGUGACUAAGCUCGCCGAGCAGUACGCUAAGGAGAACGGAACCAAGCUUCUGCU
GACUAACGACCCACUAGAAGCAGCCCACGGGGGCAACGUGCUUAUUACUGACACC
UGGAUCUCCAUGGGCCAGGAAGAAGAGAAAAAGAAGCGGCUGCAGGCGUUCCAG
GGAUAUCAGGUCACCAUGAAAACCGCCAAGGUCGCUGCCUCCGACUGGACCUUCC
UGCACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCCGU
GAUGGUGUCACUGCUCACCGACUACAGCCCGCAGCUUCAGAAGCCCAAGUUCUAG
CUCGAGCUAGUGACUGACUAGGAUCUGGUUACCACUAAACCAGCCUCAAGAACA
CCCGAAUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUCUUCAC
AUUCUAG
>mARM1845 (SEQ ID NO: 91)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAAUCUCCGCAUCCUCCUUAA
CAACGCCGCGUUUAGAAACGGCCACAACUUCAUGGUCCGGAACUUCAGAUGUGG
CCAGCCGCUUCAAAACAAGGUCCAGCUGAAGGGCCGGGAUCUUCUGACCCUGAAG
AACUUUACUGGCGAAGAGAUCAAGUACAUGCUCUGGCUCUCCGCGGACUUGAAG
UUCCGCAUUAAGCAGAAGGGGGAAUACCUUCCGCUGCUUCAAGGAAAGAGCCUC
GGCAUGAUCUUUGAGAAGCGCUCAACCAGGACCCGCCUUUCUACUGAAACUGGG
UUCGCGCUGCUCGGUGGCCACCCCUGCUUCCUGACGACCCAGGACAUCCACCUCG
GAGUGAACGAAUCCCUCACCGAUACCGCCCGGGUGUUAUCGAGCAUGGCAGAUG
CCGUGCUGGCCAGGGUGUACAAACAGUCCGAUCUGGACACUCUGGCCAAGGAGG
CGUCAAUUCCUAUUAUCAACGGCCUUAGUGACCUCUACCAUCCGAUUCAGAUCCU
GGCCGAUUACCUCACCCUGCAAGAACACUACAGCUCCCUGAAGGGUCUGACAUUG
UCCUGGAUCGGCGACGGCAACAACAUUCUCCAUUCCAUCAUGAUGUCCGCCGCAA
AAUUCGGCAUGCAUCUUCAAGCCGCCACGCCGAAGGGUUACGAGCCCGACGCUUC
CGUGACUAAGCUCGCCGAGCAGUACGCUAAGGAGAACGGAACCAAGCUUCUGCU
GACUAACGACCCACUAGAAGCAGCCCACGGGGGCAACGUGCUUAUUACUGACACC
UGGAUCUCCAUGGGCCAGGAAGAAGAGAAAAAGAAGCGGCUGCAGGCGUUCCAG
GGAUAUCAGGUCACCAUGAAAACCGCCAAGGUCGCUGCCUCCGACUGGACCUUCC
UGCACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCCGU
GAUGGUGUCACUGCUCACCGACUACAGCCCGCAGCUUCAGAAGCCCAAGUUCUAG
CUCGAGCUAGUGACUGACUAGGAUCUGGUUACCACUAAACCAGCCUCAAGAACA
CCCGAAUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUCUUCAC
AUUCUAG
>mARM1846 (SEQ ID NO: 92)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAAUCUCCGCAUCCUCCUUAA
CAACGCCGCGUUUAGAAACGGCCACAACUUCAUGGUCCGGAACUUCAGAUGUGG
CCAGCCGCUUCAAAACAAGGUCCAGCUGAAGGGCCGGGAUCUUCUGACCCUGAAG
AACUUUACUGGCGAAGAGAUCAAGUACAUGCUCUGGCUCUCCGCGGACUUGAAG
UUCCGCAUUAAGCAGAAGGGGGAAUACCUUCCGCUGCUUCAAGGAAAGAGCCUC
GGCAUGAUCUUUGAGAAGCGCUCAACCAGGACCCGCCUUUCUACUGAAACUGGG
UUCGCGCUGCUCGGUGGCCACCCCUGCUUCCUGACGACCCAGGACAUCCACCUCG
GAGUGAACGAAUCCCUCACCGAUACCGCCCGGGUGUUAUCGAGCAUGGCAGAUG
CCGUGCUGGCCAGGGUGUACAAACAGUCCGAUCUGGACACUCUGGCCAAGGAGG
CGUCAAUUCCUAUUAUCAACGGCCUUAGUGACCUCUACCAUCCGAUUCAGAUCCU
GGCCGAUUACCUCACCCUGCAAGAACACUACAGCUCCCUGAAGGGUCUGACAUUG
UCCUGGAUCGGCGACGGCAACAACAUUCUCCAUUCCAUCAUGAUGUCCGCCGCAA
AAUUCGGCAUGCAUCUUCAAGCCGCCACGCCGAAGGGUUACGAGCCCGACGCUUC
CGUGACUAAGCUCGCCGAGCAGUACGCUAAGGAGAACGGAACCAAGCUUCUGCU
GACUAACGACCCACUAGAAGCAGCCCACGGGGGCAACGUGCUUAUUACUGACACC
UGGAUCUCCAUGGGCCAGGAAGAAGAGAAAAAGAAGCGGCUGCAGGCGUUCCAG
GGAUAUCAGGUCACCAUGAAAACCGCCAAGGUCGCUGCCUCCGACUGGACCUUCC
UGCACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCCGU
GAUGGUGUCACUGCUCACCGACUACAGCCCGCAGCUUCAGAAGCCCAAGUUCUAG
CUCGAGCUAGUGACUGACUAGGAUCUGGUUACCACUAAACCAGCCUCAAGAACA
CCCGAAUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUCUUCAC
AUUCUAG
>mARM1847 (SEQ ID NO: 93)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUUUCAAUCUCCGCAUCCUCCUUAA
CAACGCCGCGUUUAGAAACGGCCACAACUUCAUGGUCCGGAACUUCAGAUGUGG
CCAGCCGCUUCAAAACAAGGUCCAGCUGAAGGGCCGGGAUCUUCUGACCCUGAAG
AACUUUACUGGCGAAGAGAUCAAGUACAUGCUCUGGCUCUCCGCGGACUUGAAG
UUCCGCAUUAAGCAGAAGGGGGAAUACCUUCCGCUGCUUCAAGGAAAGAGCCUC
GGCAUGAUCUUUGAGAAGCGCUCAACCAGGACCCGCCUUUCUACUGAAACUGGG
UUCGCGCUGCUCGGUGGCCACCCCUGCUUCCUGACGACCCAGGACAUCCACCUCG
GAGUGAACGAAUCCCUCACCGAUACCGCCCGGGUGUUAUCGAGCAUGGCAGAUG
CCGUGCUGGCCAGGGUGUACAAACAGUCCGAUCUGGACACUCUGGCCAAGGAGG
CGUCAAUUCCUAUUAUCAACGGCCUUAGUGACCUCUACCAUCCGAUUCAGAUCCU
GGCCGAUUACCUCACCCUGCAAGAACACUACAGCUCCCUGAAGGGUCUGACAUUG
UCCUGGAUCGGCGACGGCAACAACAUUCUCCAUUCCAUCAUGAUGUCCGCCGCAA
AAUUCGGCAUGCAUCUUCAAGCCGCCACGCCGAAGGGUUACGAGCCCGACGCUUC
CGUGACUAAGCUCGCCGAGCAGUACGCUAAGGAGAACGGAACCAAGCUUCUGCU
GACUAACGACCCACUAGAAGCAGCCCACGGGGGCAACGUGCUUAUUACUGACACC
UGGAUCUCCAUGGGCCAGGAAGAAGAGAAAAAGAAGCGGCUGCAGGCGUUCCAG
GGAUAUCAGGUCACCAUGAAAACCGCCAAGGUCGCUGCCUCCGACUGGACCUUCC
UGCACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCCGU
GAUGGUGUCACUGCUCACCGACUACAGCCCGCAGCUUCAGAAGCCCAAGUUCUAG
CUCGAGCUAGUGACUGACUAGGAUCUGGUUACCACUAAACCAGCCUCAAGAACA
CCCGAAUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUCUUCAC
AUUCUAG
>mARM1882 (SEQ ID NO: 94)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGGUGUUCAACCUCCGCAUCCUCCUCAA
CAACGCCGCAUUCAGAAACGGGCACAACUUCAUGGUCAGAAACUUCCGCUGCGGG
CAACCCCUACAAAACAAGGUCCAGCUCAAGGGGCGGGACCUCCUGACCCUGAAGA
ACUUCACCGGCGAAGAGAUCAAGUACAUGCUGUGGCUCUCCGCCGACCUGAAGU
UCCGCAUCAAGCAGAAGGGAGAGUACCUCCCGCUGCUGCAAGGGAAGUCGCUGG
GGAUGAUCUUCGAGAAGCGGUCAACCAGAACCCGGCUGUCAACCGAAACCGGGU
UCGCACUGCUGGGGGGACACCCGUGCUUCCUGACCACCCAAGACAUCCACCUGGG
AGUGAACGAAUCGCUGACCGACACCGCCCGCGUGCUGAGCUCAAUGGCGGACGCC
GUGCUGGCCCGCGUGUACAAGCAGUCCGACCUGGACACCCUGGCCAAGGAAGCGU
CCAUCCCGAUCAUCAACGGACUGUCCGACCUGUACCACCCGAUCCAGAUCCUGGC
AGACUACCUGACCCUGCAAGAACACUACAGCUCCCUGAAGGGCCUGACCCUGUCA
UGGAUCGGGGACGGGAACAACAUCCUGCACUCCAUAAUGAUGUCAGCCGCCAAG
UUCGGAAUGCACCUCCAAGCCGCAACCCCGAAGGGCUACGAACCGGACGCAUCAG
UGACCAAACUGGCCGAGCAGUACGCCAAGGAAAACGGCACCAAGCUCCUGCUGAC
CAACGACCCGCUGGAGGCCGCACACGGGGGGAACGUGCUGAUCACCGACACCUGG
AUCUCCAUGGGACAGGAGGAGGAAAAGAAGAAGCGGCUGCAGGCGUUCCAGGGG
UACCAGGUCACCAUGAAAACCGCGAAGGUCGCGGCAUCAGACUGGACCUUCCUGC
ACUGCCUGCCCCGGAAGCCGGAAGAGGUGGACGACGAGGUGUUCUACUCGCCGCG
CUCGCUGGUGUUCCCCGAGGCGGAGAACAGGAAGUGGACCAUCAUGGCGGUGAU
GGUCAGCCUCCUGACCGACUACUCGCCGCAGCUGCAGAAGCCGAAGUUCUGAUAA
CUCGAGCUAGUGACUGACUAGGAUCUGGUUACCACUAAACCAGCCUCAAGAACA
CCCGAAUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUCUUCAC
AUUCUAG
>mARM1883 (SEQ ID NO: 95)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGGUGUUCAACCUCCGCAUCCUCCUCAA
CAACGCCGCAUUCAGAAACGGGCACAACUUCAUGGUCAGAAACUUCCGCUGCGGG
CAACCCCUACAAAACCGGGUCCAGCUCAAGGGGCGGGACCUCCUGACCCUGAAGA
ACUUCACCGGCGAAGAGAUCAAGUACAUGCUGUGGCUCUCCGCCGACCUGAAGU
UCCGCAUCAAGCAGAAGGGAGAGUACCUCCCGCUGCUGCAAGGGAAGUCGCUGG
GGAUGAUCUUCGAGAAGCGGUCAACCAGAACCCGGCUGUCAACCGAAACCGGGU
UCGCACUGCUGGGGGGACACCCGUGCUUCCUGACCACCCAAGACAUCCACCUGGG
AGUGAACGAAUCGCUGACCGACACCGCCCGCGUGCUGAGCUCAAUGGCGGACGCC
GUGCUGGCCCGCGUGUACAAGCAGUCCGACCUGGACACCCUGGCCAAGGAAGCGU
CCAUCCCGAUCAUCAACGGACUGUCCGACCUGUACCACCCGAUCCAGAUCCUGGC
AGACUACCUGACCCUGCAAGAACACUACAGCUCCCUGAAGGGCCUGACCCUGUCA
UGGAUCGGGGACGGGAACAACAUCCUGCACUCCAUAAUGAUGUCAGCCGCCAAG
UUCGGAAUGCACCUCCAAGCCGCAACCCCGAAGGGCUACGAACCGGACGCAUCAG
UGACCAAACUGGCCGAGCAGUACGCCAAGGAAAACGGCACCAAGCUCCUGCUGAC
CAACGACCCGCUGGAGGCCGCACACGGGGGGAACGUGCUGAUCACCGACACCUGG
AUCUCCAUGGGACAGGAGGAGGAAAAGAAGAAGCGGCUGCAGGCGUUCCAGGGG
UACCAGGUCACCAUGAAAACCGCGAAGGUCGCGGCAUCAGACUGGACCUUCCUGC
ACUGCCUGCCCCGGAAGCCGGAAGAGGUGGACGACGAGGUGUUCUACUCGCCGCG
CUCGCUGGUGUUCCCCGAGGCGGAGAACAGGAAGUGGACCAUCAUGGCGGUGAU
GGUCAGCCUCCUGACCGACUACUCGCCGCAGCUGCAGAAGCCGAAGUUCUGAUAA
CUCGAGCUAGUGACUGACUAGGAUCUGGUUACCACUAAACCAGCCUCAAGAACA
CCCGAAUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUCUUCAC
AUUCUAG
>mARM1884 (SEQ ID NO: 96)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGGUGUUCAACCUCCGCAUCCUCCUCAA
CAACGCCGCAUUCAGAAACGGGCACAACUUCAUGGUCAGAAACUUCCGCUGCGGG
CAACCCCUACAAAACCGGGUCCAGCUCAAGGGGCGGGACCUCCUGACCCUGAAGA
ACUUCACCGGCGAAGAGAUCCGGUACAUGCUGUGGCUCUCCGCCGACCUGAAGUU
CCGCAUCAAGCAGAAGGGAGAGUACCUCCCGCUGCUGCAAGGGAAGUCGCUGGG
GAUGAUCUUCGAGAAGCGGUCAACCAGAACCCGGCUGUCAACCGAAACCGGGUU
CGCACUGCUGGGGGGACACCCGUGCUUCCUGACCACCCAAGACAUCCACCUGGGA
GUGAACGAAUCGCUGACCGACACCGCCCGCGUGCUGAGCUCAAUGGCGGACGCCG
UGCUGGCCCGCGUGUACAAGCAGUCCGACCUGGACACCCUGGCCAAGGAAGCGUC
CAUCCCGAUCAUCAACGGACUGUCCGACCUGUACCACCCGAUCCAGAUCCUGGCA
GACUACCUGACCCUGCAAGAACACUACAGCUCCCUGAAGGGCCUGACCCUGUCAU
GGAUCGGGGACGGGAACAACAUCCUGCACUCCAUAAUGAUGUCAGCCGCCAAGU
UCGGAAUGCACCUCCAAGCCGCAACCCCGAAGGGCUACGAACCGGACGCAUCAGU
GACCAAACUGGCCGAGCAGUACGCCAAGGAAAACGGCACCAAGCUCCUGCUGACC
AACGACCCGCUGGAGGCCGCACACGGGGGGAACGUGCUGAUCACCGACACCUGGA
UCUCCAUGGGACAGGAGGAGGAAAAGAAGAAGCGGCUGCAGGCGUUCCAGGGGU
ACCAGGUCACCAUGAAAACCGCGAAGGUCGCGGCAUCAGACUGGACCUUCCUGCA
CUGCCUGCCCCGGAAGCCGGAAGAGGUGGACGACGAGGUGUUCUACUCGCCGCGC
UCGCUGGUGUUCCCCGAGGCGGAGAACAGGAAGUGGACCAUCAUGGCGGUGAUG
GUCAGCCUCCUGACCGACUACUCGCCGCAGCUGCAGAAGCCGAAGUUCUGAUAAC
UCGAGCUAGUGACUGACUAGGAUCUGGUUACCACUAAACCAGCCUCAAGAACAC
CCGAAUGGAGUCUCUAAGCUACAUAAUACCAACUUACACUUACAAAAUGUUGUC
CCCCAAAAUGUAGCCAUUCGUAUCUGCUCCUAAUAAAAAGAAAGUUUCUUCACA
UUCUAG
>mARM1885 (SEQ ID NO: 97)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUGGUCAACCUCCGCAUCCUCCUCAA
CAACGCCGCAUUCAGAAACGGGCACAACUUCAUGGUCAGAAACUUCCGCUGCGGG
CAACCCCUACAAAACAAGGUCCAGCUCAAGGGGCGGGACCUCCUGACCCUGAAGA
ACUUCACCGGCGAAGAGAUCAAGUACAUGCUGUGGCUCUCCGCCGACCUGAAGU
UCCGCAUCAAGCAGAAGGGAGAGUACCUCCCGCUGCUGCAAGGGAAGUCGCUGG
GGAUGAUCUUCGAGAAGCGGUCAACCAGAACCCGGCUGUCAACCGAAACCGGGU
UCGCACUGCUGGGGGGACACCCGUGCUUCCUGACCACCCAAGACAUCCACCUGGG
AGUGAACGAAUCGCUGACCGACACCGCCCGCGUGCUGAGCUCAAUGGCGGACGCC
GUGCUGGCCCGCGUGUACAAGCAGUCCGACCUGGACACCCUGGCCAAGGAAGCGU
CCAUCCCGAUCAUCAACGGACUGUCCGACCUGUACCACCCGAUCCAGAUCCUGGC
AGACUACCUGACCCUGCAAGAACACUACAGCUCCCUGAAGGGCCUGACCCUGUCA
UGGAUCGGGGACGGGAACAACAUCCUGCACUCCAUAAUGAUGUCAGCCGCCAAG
UUCGGAAUGCACCUCCAAGCCGCAACCCCGAAGGGCUACGAACCGGACGCAUCAG
UGACCAAACUGGCCGAGCAGUACGCCAAGGAAAACGGCACCAAGCUCCUGCUGAC
CAACGACCCGCUGGAGGCCGCACACGGGGGGAACGUGCUGAUCACCGACACCUGG
AUCUCCAUGGGACAGGAGGAGGAAAAGAAGAAGCGGCUGCAGGCGUUCCAGGGG
UACCAGGUCACCAUGAAAACCGCGAAGGUCGCGGCAUCAGACUGGACCUUCCUGC
ACUGCCUGCCCCGGAAGCCGGAAGAGGUGGACGACGAGGUGUUCUACUCGCCGCG
CUCGCUGGUGUUCCCCGAGGCGGAGAACAGGAAGUGGACCAUCAUGGCGGUGAU
GGUCAGCCUCCUGACCGACUACUCGCCGCAGCUGCAGAAGCCGAAGUUCUGAUAA
CUCGAGCUAGUGACUGACUAGGAUCUGGUUACCACUAAACCAGCCUCAAGAACA
CCCGAAUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUCUUCAC
AUUCUAG
>mARM1886 (SEQ ID NO: 98)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUGGUCAACCUCCGCAUCCUCCUCAA
CAACGCCGCAUUCAGAAACGGGCACAACUUCAUGGUCAGAAACUUCCGCUGCGGG
CAACCCCUACAAAACCGGGUCCAGCUCAAGGGGCGGGACCUCCUGACCCUGAAGA
ACUUCACCGGCGAAGAGAUCAAGUACAUGCUGUGGCUCUCCGCCGACCUGAAGU
UCCGCAUCAAGCAGAAGGGAGAGUACCUCCCGCUGCUGCAAGGGAAGUCGCUGG
GGAUGAUCUUCGAGAAGCGGUCAACCAGAACCCGGCUGUCAACCGAAACCGGGU
UCGCACUGCUGGGGGGACACCCGUGCUUCCUGACCACCCAAGACAUCCACCUGGG
AGUGAACGAAUCGCUGACCGACACCGCCCGCGUGCUGAGCUCAAUGGCGGACGCC
GUGCUGGCCCGCGUGUACAAGCAGUCCGACCUGGACACCCUGGCCAAGGAAGCGU
CCAUCCCGAUCAUCAACGGACUGUCCGACCUGUACCACCCGAUCCAGAUCCUGGC
AGACUACCUGACCCUGCAAGAACACUACAGCUCCCUGAAGGGCCUGACCCUGUCA
UGGAUCGGGGACGGGAACAACAUCCUGCACUCCAUAAUGAUGUCAGCCGCCAAG
UUCGGAAUGCACCUCCAAGCCGCAACCCCGAAGGGCUACGAACCGGACGCAUCAG
UGACCAAACUGGCCGAGCAGUACGCCAAGGAAAACGGCACCAAGCUCCUGCUGAC
CAACGACCCGCUGGAGGCCGCACACGGGGGGAACGUGCUGAUCACCGACACCUGG
AUCUCCAUGGGACAGGAGGAGGAAAAGAAGAAGCGGCUGCAGGCGUUCCAGGGG
UACCAGGUCACCAUGAAAACCGCGAAGGUCGCGGCAUCAGACUGGACCUUCCUGC
ACUGCCUGCCCCGGAAGCCGGAAGAGGUGGACGACGAGGUGUUCUACUCGCCGCG
CUCGCUGGUGUUCCCCGAGGCGGAGAACAGGAAGUGGACCAUCAUGGCGGUGAU
GGUCAGCCUCCUGACCGACUACUCGCCGCAGCUGCAGAAGCCGAAGUUCUGAUAA
CUCGAGCUAGUGACUGACUAGGAUCUGGUUACCACUAAACCAGCCUCAAGAACA
CCCGAAUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUCUUCAC
AUUCUAG
>mARM1887 (SEQ ID NO: 99)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUGGUCAACCUCCGCAUCCUCCUCAA
CAACGCCGCAUUCAGAAACGGGCACAACUUCAUGGUCAGAAACUUCCGCUGCGGG
CAACCCCUACAAAACCGGGUCCAGCUCAAGGGGCGGGACCUCCUGACCCUGAAGA
ACUUCACCGGCGAAGAGAUCCGGUACAUGCUGUGGCUCUCCGCCGACCUGAAGUU
CCGCAUCAAGCAGAAGGGAGAGUACCUCCCGCUGCUGCAAGGGAAGUCGCUGGG
GAUGAUCUUCGAGAAGCGGUCAACCAGAACCCGGCUGUCAACCGAAACCGGGUU
CGCACUGCUGGGGGGACACCCGUGCUUCCUGACCACCCAAGACAUCCACCUGGGA
GUGAACGAAUCGCUGACCGACACCGCCCGCGUGCUGAGCUCAAUGGCGGACGCCG
UGCUGGCCCGCGUGUACAAGCAGUCCGACCUGGACACCCUGGCCAAGGAAGCGUC
CAUCCCGAUCAUCAACGGACUGUCCGACCUGUACCACCCGAUCCAGAUCCUGGCA
GACUACCUGACCCUGCAAGAACACUACAGCUCCCUGAAGGGCCUGACCCUGUCAU
GGAUCGGGGACGGGAACAACAUCCUGCACUCCAUAAUGAUGUCAGCCGCCAAGU
UCGGAAUGCACCUCCAAGCCGCAACCCCGAAGGGCUACGAACCGGACGCAUCAGU
GACCAAACUGGCCGAGCAGUACGCCAAGGAAAACGGCACCAAGCUCCUGCUGACC
AACGACCCGCUGGAGGCCGCACACGGGGGGAACGUGCUGAUCACCGACACCUGGA
UCUCCAUGGGACAGGAGGAGGAAAAGAAGAAGCGGCUGCAGGCGUUCCAGGGGU
ACCAGGUCACCAUGAAAACCGCGAAGGUCGCGGCAUCAGACUGGACCUUCCUGCA
CUGCCUGCCCCGGAAGCCGGAAGAGGUGGACGACGAGGUGUUCUACUCGCCGCGC
UCGCUGGUGUUCCCCGAGGCGGAGAACAGGAAGUGGACCAUCAUGGCGGUGAUG
GUCAGCCUCCUGACCGACUACUCGCCGCAGCUGCAGAAGCCGAAGUUCUGAUAAC
UCGAGCUAGUGACUGACUAGGAUCUGGUUACCACUAAACCAGCCUCAAGAACAC
CCGAAUGGAGUCUCUAAGCUACAUAAUACCAACUUACACUUACAAAAUGUUGUC
CCCCAAAAUGUAGCCAUUCGUAUCUGCUCCUAAUAAAAAGAAAGUUUCUUCACA
UUCUAG
>mARM1888 (SEQ ID NO: 100)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUGGUCAACCUGCGCAUCCUGCUGAA
CAACGCCGCCUUCCGCAACGGCCACAACUUCAUGGUGCGCAACUUCCGCUGCGGC
CAGCCCCUGCAGAACAAGGUGCAGCUGAAGGGCCGCGACCUGCUGACCCUGAAGA
ACUUCACCGGCGAGGAGAUCAAGUACAUGCUGUGGCUGAGCGCCGACCUGAAGU
UCCGCAUCAAGCAGAAGGGCGAGUACCUGCCCCUGCUGCAGGGCAAGAGCCUGGG
CAUGAUCUUCGAGAAGCGCAGCACCCGCACCCGCCUGAGCACCGAGACAGGCUUC
GCCCUGCUGGGCGGCCACCCCUGCUUCCUGACCACCCAGGACAUCCACCUGGGCG
UGAACGAGAGCCUGACCGACACCGCCCGCGUGCUGAGCAGCAUGGCCGACGCCGU
GCUGGCCCGCGUGUACAAGCAGAGCGACCUGGACACCCUGGCCAAGGAGGCCAGC
AUCCCCAUCAUCAACGGCCUGAGCGACCUGUACCACCCCAUCCAGAUCCUGGCCG
ACUACCUGACCCUGCAGGAGCACUACAGCAGCCUGAAGGGCCUGACCCUGAGCUG
GAUCGGCGACGGCAACAACAUCCUGCACAGCAUCAUGAUGAGCGCCGCCAAGUUC
GGCAUGCACCUGCAGGCCGCCACCCCCAAGGGCUACGAGCCCGACGCCAGCGUGA
CCAAGCUGGCCGAGCAGUACGCCAAGGAGAACGGCACCAAGCUGCUGCUGACCAA
CGACCCCCUGGAGGCCGCCCACGGCGGCAACGUGCUGAUCACCGACACCUGGAUC
AGCAUGGGCCAGGAGGAGGAGAAGAAGAAGCGCCUGCAGGCCUUCCAGGGCUAC
CAGGUGACCAUGAAGACCGCCAAGGUGGCCGCCAGCGACUGGACCUUCCUGCACU
GCCUGCCCCGCAAGCCCGAGGAGGUGGACGACGAGGUGUUCUACAGCCCCCGCAG
CCUGGUGUUCCCCGAGGCCGAGAACCGCAAGUGGACCAUCAUGGCCGUGAUGGU
GAGCCUGCUGACCGACUACAGCCCCCAGCUGCAGAAGCCCAAGUUCUGAUAACUC
GAGCUAGUGACUGACUAGGAUCUGGUUACCACUAAACCAGCCUCAAGAACACCC
GAAUGGAGUCUCUAAGCUACAUAAUACCAACUUACACUUACAAAAUGUUGUCCC
CCAAAAUGUAGCCAUUCGUAUCUGCUCCUAAUAAAAAGAAAGUUUCUUCACAUU
CUAG
>mARM1889 (SEQ ID NO: 101)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUGGUCAACCUGCGCAUCCUGCUGAA
CAACGCCGCCUUCCGCAACGGCCACAACUUCAUGGUGCGCAACUUCCGCUGCGGC
CAGCCCCUGCAGAACCGGGUGCAGCUGAAGGGCCGCGACCUGCUGACCCUGAAGA
ACUUCACCGGCGAGGAGAUCAAGUACAUGCUGUGGCUGAGCGCCGACCUGAAGU
UCCGCAUCAAGCAGAAGGGCGAGUACCUGCCCCUGCUGCAGGGCAAGAGCCUGGG
CAUGAUCUUCGAGAAGCGCAGCACCCGCACCCGCCUGAGCACCGAGACAGGCUUC
GCCCUGCUGGGCGGCCACCCCUGCUUCCUGACCACCCAGGACAUCCACCUGGGCG
UGAACGAGAGCCUGACCGACACCGCCCGCGUGCUGAGCAGCAUGGCCGACGCCGU
GCUGGCCCGCGUGUACAAGCAGAGCGACCUGGACACCCUGGCCAAGGAGGCCAGC
AUCCCCAUCAUCAACGGCCUGAGCGACCUGUACCACCCCAUCCAGAUCCUGGCCG
ACUACCUGACCCUGCAGGAGCACUACAGCAGCCUGAAGGGCCUGACCCUGAGCUG
GAUCGGCGACGGCAACAACAUCCUGCACAGCAUCAUGAUGAGCGCCGCCAAGUUC
GGCAUGCACCUGCAGGCCGCCACCCCCAAGGGCUACGAGCCCGACGCCAGCGUGA
CCAAGCUGGCCGAGCAGUACGCCAAGGAGAACGGCACCAAGCUGCUGCUGACCAA
CGACCCCCUGGAGGCCGCCCACGGCGGCAACGUGCUGAUCACCGACACCUGGAUC
AGCAUGGGCCAGGAGGAGGAGAAGAAGAAGCGCCUGCAGGCCUUCCAGGGCUAC
CAGGUGACCAUGAAGACCGCCAAGGUGGCCGCCAGCGACUGGACCUUCCUGCACU
GCCUGCCCCGCAAGCCCGAGGAGGUGGACGACGAGGUGUUCUACAGCCCCCGCAG
CCUGGUGUUCCCCGAGGCCGAGAACCGCAAGUGGACCAUCAUGGCCGUGAUGGU
GAGCCUGCUGACCGACUACAGCCCCCAGCUGCAGAAGCCCAAGUUCUGAUAACUC
GAGCUAGUGACUGACUAGGAUCUGGUUACCACUAAACCAGCCUCAAGAACACCC
GAAUGGAGUCUCUAAGCUACAUAAUACCAACUUACACUUACAAAAUGUUGUCCC
CCAAAAUGUAGCCAUUCGUAUCUGCUCCUAAUAAAAAGAAAGUUUCUUCACAUU
CUAG
>mARM1890 (SEQ ID NO: 102)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUGUCAAUCUCCGCAUCCUCCUUAA
CAACGCCGCGUUUAGAAACGGCCACAACUUCAUGGUCCGGAACUUCAGAUGUGG
CCAGCCGCUUCAAAACAAGGUCCAGCUGAAGGGCCGGGAUCUUCUGACCCUGAAG
AACUUUACUGGCGAAGAGAUCAAGUACAUGCUCUGGCUCUCCGCGGACUUGAAG
UUCCGCAUUAAGCAGAAGGGGGAAUACCUUCCGCUGCUUCAAGGAAAGAGCCUC
GGCAUGAUCUUUGAGAAGCGCUCAACCAGGACCCGCCUUUCUACUGAAACUGGG
UUCGCGCUGCUCGGUGGCCACCCCUGCUUCCUGACGACCCAGGACAUCCACCUCG
GAGUGAACGAAUCCCUCACCGAUACCGCCCGGGUGUUAUCGAGCAUGGCAGAUG
CCGUGCUGGCCAGGGUGUACAAACAGUCCGAUCUGGACACUCUGGCCAAGGAGG
CGUCAAUUCCUAUUAUCAACGGCCUUAGUGACCUCUACCAUCCGAUUCAGAUCCU
GGCCGAUUACCUCACCCUGCAAGAACACUACAGCUCCCUGAAGGGUCUGACAUUG
UCCUGGAUCGGCGACGGCAACAACAUUCUCCAUUCCAUCAUGAUGUCCGCCGCAA
AAUUCGGCAUGCAUCUUCAAGCCGCCACGCCGAAGGGUUACGAGCCCGACGCUUC
CGUGACUAAGCUCGCCGAGCAGUACGCUAAGGAGAACGGAACCAAGCUUCUGCU
GACUAACGACCCACUAGAAGCAGCCCACGGGGGCAACGUGCUUAUUACUGACACC
UGGAUCUCCAUGGGCCAGGAAGAAGAGAAAAAGAAGCGGCUGCAGGCGUUCCAG
GGAUAUCAGGUCACCAUGAAAACCGCCAAGGUCGCUGCCUCCGACUGGACCUUCC
UGCACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCCGU
GAUGGUGUCACUGCUCACCGACUACAGCCCGCAGCUUCAGAAGCCCAAGUUCUAG
CUCGAGCUAGUGACUGACUAGGAUCUGGUUACCACUAAACCAGCCUCAAGAACA
CCCGAAUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUCUUCAC
AUUCUAG
>mARM1891 (SEQ ID NO: 103)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGCUUGUCAAUCUCCGCAUCCUCCUUAA
CAACGCCGCGUUUAGAAACGGCCACAACUUCAUGGUCCGGAACUUCAGAUGUGG
CCAGCCGCUUCAAAACCGGGUCCAGCUGAAGGGCCGGGAUCUUCUGACCCUGAAG
AACUUUACUGGCGAAGAGAUCAAGUACAUGCUCUGGCUCUCCGCGGACUUGAAG
UUCCGCAUUAAGCAGAAGGGGGAAUACCUUCCGCUGCUUCAAGGAAAGAGCCUC
GGCAUGAUCUUUGAGAAGCGCUCAACCAGGACCCGCCUUUCUACUGAAACUGGG
UUCGCGCUGCUCGGUGGCCACCCCUGCUUCCUGACGACCCAGGACAUCCACCUCG
GAGUGAACGAAUCCCUCACCGAUACCGCCCGGGUGUUAUCGAGCAUGGCAGAUG
CCGUGCUGGCCAGGGUGUACAAACAGUCCGAUCUGGACACUCUGGCCAAGGAGG
CGUCAAUUCCUAUUAUCAACGGCCUUAGUGACCUCUACCAUCCGAUUCAGAUCCU
GGCCGAUUACCUCACCCUGCAAGAACACUACAGCUCCCUGAAGGGUCUGACAUUG
UCCUGGAUCGGCGACGGCAACAACAUUCUCCAUUCCAUCAUGAUGUCCGCCGCAA
AAUUCGGCAUGCAUCUUCAAGCCGCCACGCCGAAGGGUUACGAGCCCGACGCUUC
CGUGACUAAGCUCGCCGAGCAGUACGCUAAGGAGAACGGAACCAAGCUUCUGCU
GACUAACGACCCACUAGAAGCAGCCCACGGGGGCAACGUGCUUAUUACUGACACC
UGGAUCUCCAUGGGCCAGGAAGAAGAGAAAAAGAAGCGGCUGCAGGCGUUCCAG
GGAUAUCAGGUCACCAUGAAAACCGCCAAGGUCGCUGCCUCCGACUGGACCUUCC
UGCACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCCGU
GAUGGUGUCACUGCUCACCGACUACAGCCCGCAGCUUCAGAAGCCCAAGUUCUAG
CUCGAGCUAGUGACUGACUAGGAUCUGGUUACCACUAAACCAGCCUCAAGAACA
CCCGAAUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUCUUCAC
AUUCUAG
>mARM1898 (SEQ ID NO: 104)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGGGCCUUGUCAAUCUCCGCAUCCUCCU
UAACAACGCCGCGUUUAGAAACGGCCACAACUUCAUGGUCCGGAACUUCAGAUG
UGGCCAGCCGCUUCAAAACAAGGUCCAGCUGAAGGGCCGGGAUCUUCUGACCCUG
AAGAACUUUACUGGCGAAGAGAUCAAGUACAUGCUCUGGCUCUCCGCGGACUUG
AAGUUCCGCAUUAAGCAGAAGGGGGAAUACCUUCCGCUGCUUCAAGGAAAGAGC
CUCGGCAUGAUCUUUGAGAAGCGCUCAACCAGGACCCGCCUUUCUACUGAAACUG
GGUUCGCGCUGCUCGGUGGCCACCCCUGCUUCCUGACGACCCAGGACAUCCACCU
CGGAGUGAACGAAUCCCUCACCGAUACCGCCCGGGUGUUAUCGAGCAUGGCAGA
UGCCGUGCUGGCCAGGGUGUACAAACAGUCCGAUCUGGACACUCUGGCCAAGGA
GGCGUCAAUUCCUAUUAUCAACGGCCUUAGUGACCUCUACCAUCCGAUUCAGAUC
CUGGCCGAUUACCUCACCCUGCAAGAACACUACAGCUCCCUGAAGGGUCUGACAU
UGUCCUGGAUCGGCGACGGCAACAACAUUCUCCAUUCCAUCAUGAUGUCCGCCGC
AAAAUUCGGCAUGCAUCUUCAAGCCGCCACGCCGAAGGGUUACGAGCCCGACGCU
UCCGUGACUAAGCUCGCCGAGCAGUACGCUAAGGAGAACGGAACCAAGCUUCUG
CUGACUAACGACCCACUAGAAGCAGCCCACGGGGGCAACGUGCUUAUUACUGACA
CCUGGAUCUCCAUGGGCCAGGAAGAAGAGAAAAAGAAGCGGCUGCAGGCGUUCC
AGGGAUAUCAGGUCACCAUGAAAACCGCCAAGGUCGCUGCCUCCGACUGGACCUU
CCUGCACUGCCUGCCUCGCAAGCCUGAAGAAGUGGACGACGAGGUGUUCUACUCG
CCACGGAGCCUCGUGUUCCCCGAGGCCGAGAAUAGAAAGUGGACCAUCAUGGCCG
UGAUGGUGUCACUGCUCACCGACUACAGCCCGCAGCUUCAGAAGCCCAAGUUCUA
GCUCGAGCUAGUGACUGACUAGGAUCUGGUUACCACUAAACCAGCCUCAAGAAC
ACCCGAAUGGAGUCUCUAAGCUACAUAAUACCAACUUACACUUACAAAAUGUUG
UCCCCCAAAAUGUAGCCAUUCGUAUCUGCUCCUAAUAAAAAGAAAGUUUCUUCA
CAUUCUAG
>mARM1899 (SEQ ID NO: 105)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGGGCCUUGUCAAUCUCCGCAUCCUCCU
UAACAACGCCGCGUUUAGAAACGGCCACAACUUCAUGGUCCGGAACUUCAGAUG
UGGCCAGCCGCUUCAAAACCGGGUCCAGCUGAAGGGCCGGGAUCUUCUGACCCUG
AAGAACUUUACUGGCGAAGAGAUCAAGUACAUGCUCUGGCUCUCCGCGGACUUG
AAGUUCCGCAUUAAGCAGAAGGGGGAAUACCUUCCGCUGCUUCAAGGAAAGAGC
CUCGGCAUGAUCUUUGAGAAGCGCUCAACCAGGACCCGCCUUUCUACUGAAACUG
GGUUCGCGCUGCUCGGUGGCCACCCCUGCUUCCUGACGACCCAGGACAUCCACCU
CGGAGUGAACGAAUCCCUCACCGAUACCGCCCGGGUGUUAUCGAGCAUGGCAGA
UGCCGUGCUGGCCAGGGUGUACAAACAGUCCGAUCUGGACACUCUGGCCAAGGA
GGCGUCAAUUCCUAUUAUCAACGGCCUUAGUGACCUCUACCAUCCGAUUCAGAUC
CUGGCCGAUUACCUCACCCUGCAAGAACACUACAGCUCCCUGAAGGGUCUGACAU
UGUCCUGGAUCGGCGACGGCAACAACAUUCUCCAUUCCAUCAUGAUGUCCGCCGC
AAAAUUCGGCAUGCAUCUUCAAGCCGCCACGCCGAAGGGUUACGAGCCCGACGCU
UCCGUGACUAAGCUCGCCGAGCAGUACGCUAAGGAGAACGGAACCAAGCUUCUG
CUGACUAACGACCCACUAGAAGCAGCCCACGGGGGCAACGUGCUUAUUACUGACA
CCUGGAUCUCCAUGGGCCAGGAAGAAGAGAAAAAGAAGCGGCUGCAGGCGUUCC
AGGGAUAUCAGGUCACCAUGAAAACCGCCAAGGUCGCUGCCUCCGACUGGACCUU
CCUGCACUGCCUGCCUCGCAAGCCUGAAGAAGUGGACGACGAGGUGUUCUACUCG
CCACGGAGCCUCGUGUUCCCCGAGGCCGAGAAUAGAAAGUGGACCAUCAUGGCCG
UGAUGGUGUCACUGCUCACCGACUACAGCCCGCAGCUUCAGAAGCCCAAGUUCUA
GCUCGAGCUAGUGACUGACUAGGAUCUGGUUACCACUAAACCAGCCUCAAGAAC
ACCCGAAUGGAGUCUCUAAGCUACAUAAUACCAACUUACACUUACAAAAUGUUG
UCCCCCAAAAUGUAGCCAUUCGUAUCUGCUCCUAAUAAAAAGAAAGUUUCUUCA
CAUUCUAG
>mARM1900 (SEQ ID NO: 106)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGGGCGGACUUGUCAAUCUCCGCAUCCU
CCUUAACAACGCCGCGUUUAGAAACGGCCACAACUUCAUGGUCCGGAACUUCAGA
UGUGGCCAGCCGCUUCAAAACAAGGUCCAGCUGAAGGGCCGGGAUCUUCUGACCC
UGAAGAACUUUACUGGCGAAGAGAUCAAGUACAUGCUCUGGCUCUCCGCGGACU
UGAAGUUCCGCAUUAAGCAGAAGGGGGAAUACCUUCCGCUGCUUCAAGGAAAGA
GCCUCGGCAUGAUCUUUGAGAAGCGCUCAACCAGGACCCGCCUUUCUACUGAAAC
UGGGUUCGCGCUGCUCGGUGGCCACCCCUGCUUCCUGACGACCCAGGACAUCCAC
CUCGGAGUGAACGAAUCCCUCACCGAUACCGCCCGGGUGUUAUCGAGCAUGGCAG
AUGCCGUGCUGGCCAGGGUGUACAAACAGUCCGAUCUGGACACUCUGGCCAAGG
AGGCGUCAAUUCCUAUUAUCAACGGCCUUAGUGACCUCUACCAUCCGAUUCAGA
UCCUGGCCGAUUACCUCACCCUGCAAGAACACUACAGCUCCCUGAAGGGUCUGAC
AUUGUCCUGGAUCGGCGACGGCAACAACAUUCUCCAUUCCAUCAUGAUGUCCGCC
GCAAAAUUCGGCAUGCAUCUUCAAGCCGCCACGCCGAAGGGUUACGAGCCCGACG
CUUCCGUGACUAAGCUCGCCGAGCAGUACGCUAAGGAGAACGGAACCAAGCUUC
UGCUGACUAACGACCCACUAGAAGCAGCCCACGGGGGCAACGUGCUUAUUACUG
ACACCUGGAUCUCCAUGGGCCAGGAAGAAGAGAAAAAGAAGCGGCUGCAGGCGU
UCCAGGGAUAUCAGGUCACCAUGAAAACCGCCAAGGUCGCUGCCUCCGACUGGAC
CUUCCUGCACUGCCUGCCUCGCAAGCCUGAAGAAGUGGACGACGAGGUGUUCUAC
UCGCCACGGAGCCUCGUGUUCCCCGAGGCCGAGAAUAGAAAGUGGACCAUCAUG
GCCGUGAUGGUGUCACUGCUCACCGACUACAGCCCGCAGCUUCAGAAGCCCAAGU
UCUAGCUCGAGCUAGUGACUGACUAGGAUCUGGUUACCACUAAACCAGCCUCAA
GAACACCCGAAUGGAGUCUCUAAGCUACAUAAUACCAACUUACACUUACAAAAU
GUUGUCCCCCAAAAUGUAGCCAUUCGUAUCUGCUCCUAAUAAAAAGAAAGUUUC
UUCACAUUCUAG
>mARM1903 (SEQ ID NO: 107)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCAUGGCCCUUUUCAAUCUCCGCAUCCUCCUUAA
CAACGCCGCGUUUAGAAACGGCCACAACUUCAUGGUCCGGAACUUCAGAUGUGG
CCAGCCGCUUCAAGGCAAGGUCCAGCUGAAGGGCCGGGAUCUUCUGACCCUGAAG
AACUUUACUGGCGAAGAGAUCAAGUACAUGCUCUGGCUCUCCGCGGACUUGAAG
UUCCGCAUUAAGCAGAAGGGGGAAUACCUUCCGCUGCUUCAAGGAAAGAGCCUC
GGCAUGAUCUUUGAGAAGCGCUCAACCAGGACCCGCCUUUCUACUGAAACUGGG
UUCGCGCUGCUCGGUGGCCACCCCUGCUUCCUGACGACCCAGGACAUCCACCUCG
GAGUGAACGAAUCCCUCACCGAUACCGCCCGGGUGUUAUCGAGCAUGGCAGAUG
CCGUGCUGGCCAGGGUGUACAAACAGUCCGAUCUGGACACUCUGGCCAAGGAGG
CGUCAAUUCCUAUUAUCAACGGCCUUAGUGACCUCUACCAUCCGAUUCAGAUCCU
GGCCGAUUACCUCACCCUGCAAGAACACUACAGCUCCCUGAAGGGUCUGACAUUG
UCCUGGAUCGGCGACGGCAACAACAUUCUCCAUUCCAUCAUGAUGUCCGCCGCAA
AAUUCGGCAUGCAUCUUCAAGCCGCCACGCCGAAGGGUUACGAGCCCGACGCUUC
CGUGACUAAGCUCGCCGAGCAGUACGCUAAGGAGAACGGAACCAAGCUUCUGCU
GACUAACGACCCACUAGAAGCAGCCCACGGGGGCAACGUGCUUAUUACUGACACC
UGGAUCUCCAUGGGCCAGGAAGAAGAGAAAAAGAAGCGGCUGCAGGCGUUCCAG
GGAUAUCAGGUCACCAUGAAAACCGCCAAGGUCGCUGCCUCCGACUGGACCUUCC
UGCACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCCGU
GAUGGUGUCACUGCUCACCGACUACAGCCCGCAGCUUCAGAAGCCCAAGUUCUAG
AUAAGUGAACUCGAGCUAGUGACUGACUAGGAUCUGGUUACCACUAAACCAGCC
UCAAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAAAG
UUUCUUCACAUUCUAG
>mARM1904 (SEQ ID NO: 108)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCAUGGCCCUUUUCAAUCUCCGCAUCCUCCUUAA
CAACGCCGCGUUUAGAAACGGCCACAACUUCAUGGUCCGGAACUUCAGAUGUGG
CCAGCCGCUUCAAGGCCGGGUCCAGCUGAAGGGCCGGGAUCUUCUGACCCUGAAG
AACUUUACUGGCGAAGAGAUCAAGUACAUGCUCUGGCUCUCCGCGGACUUGAAG
UUCCGCAUUAAGCAGAAGGGGGAAUACCUUCCGCUGCUUCAAGGAAAGAGCCUC
GGCAUGAUCUUUGAGAAGCGCUCAACCAGGACCCGCCUUUCUACUGAAACUGGG
UUCGCGCUGCUCGGUGGCCACCCCUGCUUCCUGACGACCCAGGACAUCCACCUCG
GAGUGAACGAAUCCCUCACCGAUACCGCCCGGGUGUUAUCGAGCAUGGCAGAUG
CCGUGCUGGCCAGGGUGUACAAACAGUCCGAUCUGGACACUCUGGCCAAGGAGG
CGUCAAUUCCUAUUAUCAACGGCCUUAGUGACCUCUACCAUCCGAUUCAGAUCCU
GGCCGAUUACCUCACCCUGCAAGAACACUACAGCUCCCUGAAGGGUCUGACAUUG
UCCUGGAUCGGCGACGGCAACAACAUUCUCCAUUCCAUCAUGAUGUCCGCCGCAA
AAUUCGGCAUGCAUCUUCAAGCCGCCACGCCGAAGGGUUACGAGCCCGACGCUUC
CGUGACUAAGCUCGCCGAGCAGUACGCUAAGGAGAACGGAACCAAGCUUCUGCU
GACUAACGACCCACUAGAAGCAGCCCACGGGGGCAACGUGCUUAUUACUGACACC
UGGAUCUCCAUGGGCCAGGAAGAAGAGAAAAAGAAGCGGCUGCAGGCGUUCCAG
GGAUAUCAGGUCACCAUGAAAACCGCCAAGGUCGCUGCCUCCGACUGGACCUUCC
UGCACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCCGU
GAUGGUGUCACUGCUCACCGACUACAGCCCGCAGCUUCAGAAGCCCAAGUUCUAG
AUAAGUGAACUCGAGCUAGUGACUGACUAGGAUCUGGUUACCACUAAACCAGCC
UCAAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAAAG
UUUCUUCACAUUCUAG
>mARM1905 (SEQ ID NO: 109)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCAUGGCCCUUUUCAAUCUCCGCAUCCUCCUUAA
CAACGCCGCGUUUAGAAACGGCCACAACUUCAUGGUCCGGAACUUCAGAUGUGG
CCAGCCGCUUCAAGGCCGGGUCCAGCUGAAGGGCCGGGAUCUUCUGACCCUGAAG
AACUUUACUGGCGAAGAGAUCAGGUACAUGCUCUGGCUCUCCGCGGACUUGAAG
UUCCGCAUUAAGCAGAAGGGGGAAUACCUUCCGCUGCUUCAAGGAAAGAGCCUC
GGCAUGAUCUUUGAGAAGCGCUCAACCAGGACCCGCCUUUCUACUGAAACUGGG
UUCGCGCUGCUCGGUGGCCACCCCUGCUUCCUGACGACCCAGGACAUCCACCUCG
GAGUGAACGAAUCCCUCACCGAUACCGCCCGGGUGUUAUCGAGCAUGGCAGAUG
CCGUGCUGGCCAGGGUGUACAAACAGUCCGAUCUGGACACUCUGGCCAAGGAGG
CGUCAAUUCCUAUUAUCAACGGCCUUAGUGACCUCUACCAUCCGAUUCAGAUCCU
GGCCGAUUACCUCACCCUGCAAGAACACUACAGCUCCCUGAAGGGUCUGACAUUG
UCCUGGAUCGGCGACGGCAACAACAUUCUCCAUUCCAUCAUGAUGUCCGCCGCAA
AAUUCGGCAUGCAUCUUCAAGCCGCCACGCCGAAGGGUUACGAGCCCGACGCUUC
CGUGACUAAGCUCGCCGAGCAGUACGCUAAGGAGAACGGAACCAAGCUUCUGCU
GACUAACGACCCACUAGAAGCAGCCCACGGGGGCAACGUGCUUAUUACUGACACC
UGGAUCUCCAUGGGCCAGGAAGAAGAGAAAAAGAAGCGGCUGCAGGCGUUCCAG
GGAUAUCAGGUCACCAUGAAAACCGCCAAGGUCGCUGCCUCCGACUGGACCUUCC
UGCACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCCGU
GAUGGUGUCACUGCUCACCGACUACAGCCCGCAGCUUCAGAAGCCCAAGUUCUAG
AUAAGUGAACUCGAGCUAGUGACUGACUAGGAUCUGGUUACCACUAAACCAGCC
UCAAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAAAG
UUUCUUCACAUUCUAG
>mARM1906 (SEQ ID NO: 110)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCAUGGCCCUUGUCAAUCUCCGCAUCCUCCUUAA
CAACGCCGCGUUUAGAAACGGCCACAACUUCAUGGUCCGGAACUUCAGAUGUGG
CCAGCCGCUUCAAGGCAGGGUCCAGCUGAAGGGCCGGGAUCUUCUGACCCUGAAG
AACUUUACUGGCGAAGAGAUCAAGUACAUGCUCUGGCUCUCCGCGGACUUGAAG
UUCCGCAUUAAGCAGAAGGGGGAAUACCUUCCGCUGCUUCAAGGAAAGAGCCUC
GGCAUGAUCUUUGAGAAGCGCUCAACCAGGACCCGCCUUUCUACUGAAACUGGG
UUCGCGCUGCUCGGUGGCCACCCCUGCUUCCUGACGACCCAGGACAUCCACCUCG
GAGUGAACGAAUCCCUCACCGAUACCGCCCGGGUGUUAUCGAGCAUGGCAGAUG
CCGUGCUGGCCAGGGUGUACAAACAGUCCGAUCUGGACACUCUGGCCAAGGAGG
CGUCAAUUCCUAUUAUCAACGGCCUUAGUGACCUCUACCAUCCGAUUCAGAUCCU
GGCCGAUUACCUCACCCUGCAAGAACACUACAGCUCCCUGAAGGGUCUGACAUUG
UCCUGGAUCGGCGACGGCAACAACAUUCUCCAUUCCAUCAUGAUGUCCGCCGCAA
AAUUCGGCAUGCAUCUUCAAGCCGCCACGCCGAAGGGUUACGAGCCCGACGCUUC
CGUGACUAAGCUCGCCGAGCAGUACGCUAAGGAGAACGGAACCAAGCUUCUGCU
GACUAACGACCCACUAGAAGCAGCCCACGGGGGCAACGUGCUUAUUACUGACACC
UGGAUCUCCAUGGGCCAGGAAGAAGAGAAAAAGAAGCGGCUGCAGGCGUUCCAG
GGAUAUCAGGUCACCAUGAAAACCGCCAAGGUCGCUGCCUCCGACUGGACCUUCC
UGCACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCCGU
GAUGGUGUCACUGCUCACCGACUACAGCCCGCAGCUUCAGAAGCCCAAGUUCUAA
GUGAAUAGACUCGAGCUAGUGACUGACUAGGAUCUGGUUACCACUAAACCAGCC
UCAAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAAAG
UUUCUUCACAUUCUAG
>mARM1907 (SEQ ID NO: 111)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCAUGGCCCUUUUCAAUCUCCGCAUCCUCCUUAA
CAACGCCGCGUUUAGAAACGGCCACAACUUCAUGGUCCGGAACUUCAGAUGUGG
CCAGCCGCUUCAAGUCAAGGUCCAGCUGAAGGGCCGGGAUCUUCUGACCCUGAAG
AACUUUACUGGCGAAGAGAUCAAGUACAUGCUCUGGCUCUCCGCGGACUUGAAG
UUCCGCAUUAAGCAGAAGGGGGAAUACCUUCCGCUGCUUCAAGGAAAGAGCCUC
GGCAUGAUCUUUGAGAAGCGCUCAACCAGGACCCGCCUUUCUACUGAAACUGGG
UUCGCGCUGCUCGGUGGCCACCCCUGCUUCCUGACGACCCAGGACAUCCACCUCG
GAGUGAACGAAUCCCUCACCGAUACCGCCCGGGUGUUAUCGAGCAUGGCAGAUG
CCGUGCUGGCCAGGGUGUACAAACAGUCCGAUCUGGACACUCUGGCCAAGGAGG
CGUCAAUUCCUAUUAUCAACGGCCUUAGUGACCUCUACCAUCCGAUUCAGAUCCU
GGCCGAUUACCUCACCCUGCAAGAACACUACAGCUCCCUGAAGGGUCUGACAUUG
UCCUGGAUCGGCGACGGCAACAACAUUCUCCAUUCCAUCAUGAUGUCCGCCGCAA
AAUUCGGCAUGCAUCUUCAAGCCGCCACGCCGAAGGGUUACGAGCCCGACGCUUC
CGUGACUAAGCUCGCCGAGCAGUACGCUAAGGAGAACGGAACCAAGCUUCUGCU
GACUAACGACCCACUAGAAGCAGCCCACGGGGGCAACGUGCUUAUUACUGACACC
UGGAUCUCCAUGGGCCAGGAAGAAGAGAAAAAGAAGCGGCUGCAGGCGUUCCAG
GGAUAUCAGGUCACCAUGAAAACCGCCAAGGUCGCUGCCUCCGACUGGACCUUCC
UGCACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCCGU
GAUGGUGUCACUGCUCACCGACUACAGCCCGCAGCUUCAGAAGCCCAAGUUCUAG
AUAAGUGAACUCGAGCUAGUGACUGACUAGGAUCUGGUUACCACUAAACCAGCC
UCAAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAAAG
UUUCUUCACAUUCUAG
>mARM1908 (SEQ ID NO: 112)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCAUGGCCCUUUUCAAUCUCCGCAUCCUCCUUAA
CAACGCCGCGUUUAGAAACGGCCACAACUUCAUGGUCCGGAACUUCAGAUGUGG
CCAGCCGCUUCAAGUCAGGGUCCAGCUGAAGGGCCGGGAUCUUCUGACCCUGAAG
AACUUUACUGGCGAAGAGAUCAAGUACAUGCUCUGGCUCUCCGCGGACUUGAAG
UUCCGCAUUAAGCAGAAGGGGGAAUACCUUCCGCUGCUUCAAGGAAAGAGCCUC
GGCAUGAUCUUUGAGAAGCGCUCAACCAGGACCCGCCUUUCUACUGAAACUGGG
UUCGCGCUGCUCGGUGGCCACCCCUGCUUCCUGACGACCCAGGACAUCCACCUCG
GAGUGAACGAAUCCCUCACCGAUACCGCCCGGGUGUUAUCGAGCAUGGCAGAUG
CCGUGCUGGCCAGGGUGUACAAACAGUCCGAUCUGGACACUCUGGCCAAGGAGG
CGUCAAUUCCUAUUAUCAACGGCCUUAGUGACCUCUACCAUCCGAUUCAGAUCCU
GGCCGAUUACCUCACCCUGCAAGAACACUACAGCUCCCUGAAGGGUCUGACAUUG
UCCUGGAUCGGCGACGGCAACAACAUUCUCCAUUCCAUCAUGAUGUCCGCCGCAA
AAUUCGGCAUGCAUCUUCAAGCCGCCACGCCGAAGGGUUACGAGCCCGACGCUUC
CGUGACUAAGCUCGCCGAGCAGUACGCUAAGGAGAACGGAACCAAGCUUCUGCU
GACUAACGACCCACUAGAAGCAGCCCACGGGGGCAACGUGCUUAUUACUGACACC
UGGAUCUCCAUGGGCCAGGAAGAAGAGAAAAAGAAGCGGCUGCAGGCGUUCCAG
GGAUAUCAGGUCACCAUGAAAACCGCCAAGGUCGCUGCCUCCGACUGGACCUUCC
UGCACUGCCUGCCUCGCAAGCCUGAAGAAGUGGACGACGAGGUGUUCUACUCGCC
ACGGAGCCUCGUGUUCCCCGAGGCCGAGAAUAGAAAGUGGACCAUCAUGGCCGU
GAUGGUGUCACUGCUCACCGACUACAGCCCGCAGCUUCAGAAGCCCAAGUUCUAG
AUAAGUGAACUCGAGCUAGUGACUGACUAGGAUCUGGUUACCACUAAACCAGCC
UCAAGAACACCCGAAUGGAGUCUCUAAGCUACAUAAUACCAACUUACACUUACA
AAAUGUUGUCCCCCAAAAUGUAGCCAUUCGUAUCUGCUCCUAAUAAAAAGAAAG
UUUCUUCACAUUCUAG
>mARM1915 (SEQ ID NO: 113)
GGCAGAAAAAUUUGCUACAUUGUUUCACAAACUUCAAAUAUUAUUCAUUUAUUU
AGAUCUAUUAUUACAUCAAAACAAAAAGCCGCCACCAUGCUGUUCAACCUGCGC
AUCCUGCUGAACAACGCCGCCUUCCGCAACGGCCACAACUUCAUGGUGCGCAACU
UCCGCUGCGGCCAGCCCCUGCAGAACAAGGUGCAGCUGAAGGGCCGCGACCUGCU
GACCCUGAAGAACUUCACCGGCGAGGAGAUCAAGUACAUGCUGUGGCUGAGCGC
CGACCUGAAGUUCCGCAUCAAGCAGAAGGGCGAGUACCUGCCCCUGCUGCAGGGC
AAGAGCCUGGGCAUGAUCUUCGAGAAGCGCAGCACCCGCACCCGCCUGAGCACCG
AGACAGGCUUCGCCCUGCUGGGCGGCCACCCCUGCUUCCUGACCACCCAGGACAU
CCACCUGGGCGUGAACGAGAGCCUGACCGACACCGCCCGCGUGCUGAGCAGCAUG
GCCGACGCCGUGCUGGCCCGCGUGUACAAGCAGAGCGACCUGGACACCCUGGCCA
AGGAGGCCAGCAUCCCCAUCAUCAACGGCCUGAGCGACCUGUACCACCCCAUCCA
GAUCCUGGCCGACUACCUGACCCUGCAGGAGCACUACAGCAGCCUGAAGGGCCUG
ACCCUGAGCUGGAUCGGCGACGGCAACAACAUCCUGCACAGCAUCAUGAUGAGCG
CCGCCAAGUUCGGCAUGCACCUGCAGGCCGCCACCCCCAAGGGCUACGAGCCCGA
CGCCAGCGUGACCAAGCUGGCCGAGCAGUACGCCAAGGAGAACGGCACCAAGCUG
CUGCUGACCAACGACCCCCUGGAGGCCGCCCACGGCGGCAACGUGCUGAUCACCG
ACACCUGGAUCAGCAUGGGCCAGGAGGAGGAGAAGAAGAAGCGCCUGCAGGCCU
UCCAGGGCUACCAGGUGACCAUGAAGACCGCCAAGGUGGCCGCCAGCGACUGGAC
CUUCCUGCACUGCCUGCCCCGCAAGCCCGAGGAGGUGGACGACGAGGUGUUCUAC
AGCCCCCGCAGCCUGGUGUUCCCCGAGGCCGAGAACCGCAAGUGGACCAUCAUGG
CCGUGAUGGUGAGCCUGCUGACCGACUACAGCCCCCAGCUGCAGAAGCCCAAGUU
CUGAGGUCUCUAGUAAUGAGCUGGAGCCUCGGUAGCCGUUCCUCCUGCCCGCUGG
GCCUCCCAACGGGCCCUCCUCCCCUCCUUGCACCGGCCCUUCCUGGUCUUUGAAU
AAAGUCUGAGUGGGCAUCUAG
>mARM1916 (SEQ ID NO: 114)
GGCAGAAAAAUUUGCUACAUUGUUUCACAAACUUCAAAUAUUAUUCAUUUAUUU
AGAUCUAUUAUUACAUCAAAACAAAAAGCCGCCACCAUGGGAGUAUUCAACCUG
CGCAUCCUGCUGAACAACGCCGCCUUCCGCAACGGCCACAACUUCAUGGUGCGCA
ACUUCCGCUGCGGCCAGCCCCUGCAGAACAAGGUGCAGCUGAAGGGCCGCGACCU
GCUGACCCUGAAGAACUUCACCGGCGAGGAGAUCAAGUACAUGCUGUGGCUGAG
CGCCGACCUGAAGUUCCGCAUCAAGCAGAAGGGCGAGUACCUGCCCCUGCUGCAG
GGCAAGAGCCUGGGCAUGAUCUUCGAGAAGCGCAGCACCCGCACCCGCCUGAGCA
CCGAGACAGGCUUCGCCCUGCUGGGCGGCCACCCCUGCUUCCUGACCACCCAGGA
CAUCCACCUGGGCGUGAACGAGAGCCUGACCGACACCGCCCGCGUGCUGAGCAGC
AUGGCCGACGCCGUGCUGGCCCGCGUGUACAAGCAGAGCGACCUGGACACCCUGG
CCAAGGAGGCCAGCAUCCCCAUCAUCAACGGCCUGAGCGACCUGUACCACCCCAU
CCAGAUCCUGGCCGACUACCUGACCCUGCAGGAGCACUACAGCAGCCUGAAGGGC
CUGACCCUGAGCUGGAUCGGCGACGGCAACAACAUCCUGCACAGCAUCAUGAUGA
GCGCCGCCAAGUUCGGCAUGCACCUGCAGGCCGCCACCCCCAAGGGCUACGAGCC
CGACGCCAGCGUGACCAAGCUGGCCGAGCAGUACGCCAAGGAGAACGGCACCAAG
CUGCUGCUGACCAACGACCCCCUGGAGGCCGCCCACGGCGGCAACGUGCUGAUCA
CCGACACCUGGAUCAGCAUGGGCCAGGAGGAGGAGAAGAAGAAGCGCCUGCAGG
CCUUCCAGGGCUACCAGGUGACCAUGAAGACCGCCAAGGUGGCCGCCAGCGACUG
GACCUUCCUGCACUGCCUGCCCCGCAAGCCCGAGGAGGUGGACGACGAGGUGUUC
UACAGCCCCCGCAGCCUGGUGUUCCCCGAGGCCGAGAACCGCAAGUGGACCAUCA
UGGCCGUGAUGGUGAGCCUGCUGACCGACUACAGCCCCCAGCUGCAGAAGCCCAA
GUUCUGAGGUCUCUAGUAAUGAGCUGGAGCCUCGGUAGCCGUUCCUCCUGCCCGC
UGGGCCUCCCAACGGGCCCUCCUCCCCUCCUUGCACCGGCCCUUCCUGGUCUUUG
AAUAAAGUCUGAGUGGGCAUCUAG
>mARM1917 (SEQ ID NO: 115)
GGCAGAAAAAUUUGCUACAUUGUUUCACAAACUUCAAAUAUUAUUCAUUUAUUU
AGAUCUAUUAUUACAUCAAAACAAAAAGCCGCCACCAUGGGAGUAUUCAACCUG
CGCAUCCUGCUGAACAACGCCGCCUUCCGCAACGGCCACAACUUCAUGGUGCGCA
ACUUCCGCUGCGGCCAGCCCCUGCAGAACCGGGUGCAGCUGAAGGGCCGCGACCU
GCUGACCCUGAAGAACUUCACCGGCGAGGAGAUCCGGUACAUGCUGUGGCUGAG
CGCCGACCUGAAGUUCCGCAUCAAGCAGAAGGGCGAGUACCUGCCCCUGCUGCAG
GGCAAGAGCCUGGGCAUGAUCUUCGAGAAGCGCAGCACCCGCACCCGCCUGAGCA
CCGAGACAGGCUUCGCCCUGCUGGGCGGCCACCCCUGCUUCCUGACCACCCAGGA
CAUCCACCUGGGCGUGAACGAGAGCCUGACCGACACCGCCCGCGUGCUGAGCAGC
AUGGCCGACGCCGUGCUGGCCCGCGUGUACAAGCAGAGCGACCUGGACACCCUGG
CCAAGGAGGCCAGCAUCCCCAUCAUCAACGGCCUGAGCGACCUGUACCACCCCAU
CCAGAUCCUGGCCGACUACCUGACCCUGCAGGAGCACUACAGCAGCCUGAAGGGC
CUGACCCUGAGCUGGAUCGGCGACGGCAACAACAUCCUGCACAGCAUCAUGAUGA
GCGCCGCCAAGUUCGGCAUGCACCUGCAGGCCGCCACCCCCAAGGGCUACGAGCC
CGACGCCAGCGUGACCAAGCUGGCCGAGCAGUACGCCAAGGAGAACGGCACCAAG
CUGCUGCUGACCAACGACCCCCUGGAGGCCGCCCACGGCGGCAACGUGCUGAUCA
CCGACACCUGGAUCAGCAUGGGCCAGGAGGAGGAGAAGAAGAAGCGCCUGCAGG
CCUUCCAGGGCUACCAGGUGACCAUGAAGACCGCCAAGGUGGCCGCCAGCGACUG
GACCUUCCUGCACUGCCUGCCCCGCAAGCCCGAGGAGGUGGACGACGAGGUGUUC
UACAGCCCCCGCAGCCUGGUGUUCCCCGAGGCCGAGAACCGCAAGUGGACCAUCA
UGGCCGUGAUGGUGAGCCUGCUGACCGACUACAGCCCCCAGCUGCAGAAGCCCAA
GUUCUGAGGUCUCUAGUAAUGAGCUGGAGCCUCGGUAGCCGUUCCUCCUGCCCGC
UGGGCCUCCCAACGGGCCCUCCUCCCCUCCUUGCACCGGCCCUUCCUGGUCUUUG
AAUAAAGUCUGAGUGGGCAUCUAG
>mARM1918 (SEQ ID NO: 116)
GGCAGAAAAAUUUGCUACAUUGUUUCACAAACUUCAAAUAUUAUUCAUUUAUUU
AGAUCUAUUAUUACAUCAAAACAAAAAGCCGCCACCAUGCUGGUAUUCAACCUG
CGCAUCCUGCUGAACAACGCCGCCUUCCGCAACGGCCACAACUUCAUGGUGCGCA
ACUUCCGCUGCGGCCAGCCCCUGCAGAACCGGGUGCAGCUGAAGGGCCGCGACCU
GCUGACCCUGAAGAACUUCACCGGCGAGGAGAUCCGGUACAUGCUGUGGCUGAG
CGCCGACCUGAAGUUCCGCAUCAAGCAGAAGGGCGAGUACCUGCCCCUGCUGCAG
GGCAAGAGCCUGGGCAUGAUCUUCGAGAAGCGCAGCACCCGCACCCGCCUGAGCA
CCGAGACAGGCUUCGCCCUGCUGGGCGGCCACCCCUGCUUCCUGACCACCCAGGA
CAUCCACCUGGGCGUGAACGAGAGCCUGACCGACACCGCCCGCGUGCUGAGCAGC
AUGGCCGACGCCGUGCUGGCCCGCGUGUACAAGCAGAGCGACCUGGACACCCUGG
CCAAGGAGGCCAGCAUCCCCAUCAUCAACGGCCUGAGCGACCUGUACCACCCCAU
CCAGAUCCUGGCCGACUACCUGACCCUGCAGGAGCACUACAGCAGCCUGAAGGGC
CUGACCCUGAGCUGGAUCGGCGACGGCAACAACAUCCUGCACAGCAUCAUGAUGA
GCGCCGCCAAGUUCGGCAUGCACCUGCAGGCCGCCACCCCCAAGGGCUACGAGCC
CGACGCCAGCGUGACCAAGCUGGCCGAGCAGUACGCCAAGGAGAACGGCACCAAG
CUGCUGCUGACCAACGACCCCCUGGAGGCCGCCCACGGCGGCAACGUGCUGAUCA
CCGACACCUGGAUCAGCAUGGGCCAGGAGGAGGAGAAGAAGAAGCGCCUGCAGG
CCUUCCAGGGCUACCAGGUGACCAUGAAGACCGCCAAGGUGGCCGCCAGCGACUG
GACCUUCCUGCACUGCCUGCCCCGCAAGCCCGAGGAGGUGGACGACGAGGUGUUC
UACAGCCCCCGCAGCCUGGUGUUCCCCGAGGCCGAGAACCGCAAGUGGACCAUCA
UGGCCGUGAUGGUGAGCCUGCUGACCGACUACAGCCCCCAGCUGCAGAAGCCCAA
GUUCUGAGGUCUCUAGUAAUGAGCUGGAGCCUCGGUAGCCGUUCCUCCUGCCCGC
UGGGCCUCCCAACGGGCCCUCCUCCCCUCCUUGCACCGGCCCUUCCUGGUCUUUG
AAUAAAGUCUGAGUGGGCAUCUAG
>mARM1919 (SEQ ID NO: 117)
AUUAUUACAUCAAAACAAAAAGCCGCCACCAUGGGAGUAUUCAACCUGCGCAUC
CUGCUGAACAACGCCGCCUUCCGCAACGGCCACAACUUCAUGGUGCGCAACUUCC
GCUGCGGCCAGCCCCUGCAGAACAAGGUGCAGCUGAAGGGCCGCGACCUGCUGAC
CCUGAAGAACUUCACCGGCGAGGAGAUCAAGUACAUGCUGUGGCUGAGCGCCGA
CCUGAAGUUCCGCAUCAAGCAGAAGGGCGAGUACCUGCCCCUGCUGCAGGGCAAG
AGCCUGGGCAUGAUCUUCGAGAAGCGCAGCACCCGCACCCGCCUGAGCACCGAGA
CAGGCUUCGCCCUGCUGGGCGGCCACCCCUGCUUCCUGACCACCCAGGACAUCCA
CCUGGGCGUGAACGAGAGCCUGACCGACACCGCCCGCGUGCUGAGCAGCAUGGCC
GACGCCGUGCUGGCCCGCGUGUACAAGCAGAGCGACCUGGACACCCUGGCCAAGG
AGGCCAGCAUCCCCAUCAUCAACGGCCUGAGCGACCUGUACCACCCCAUCCAGAU
CCUGGCCGACUACCUGACCCUGCAGGAGCACUACAGCAGCCUGAAGGGCCUGACC
CUGAGCUGGAUCGGCGACGGCAACAACAUCCUGCACAGCAUCAUGAUGAGCGCCG
CCAAGUUCGGCAUGCACCUGCAGGCCGCCACCCCCAAGGGCUACGAGCCCGACGC
CAGCGUGACCAAGCUGGCCGAGCAGUACGCCAAGGAGAACGGCACCAAGCUGCUG
CUGACCAACGACCCCCUGGAGGCCGCCCACGGCGGCAACGUGCUGAUCACCGACA
CCUGGAUCAGCAUGGGCCAGGAGGAGGAGAAGAAGAAGCGCCUGCAGGCCUUCC
AGGGCUACCAGGUGACCAUGAAGACCGCCAAGGUGGCCGCCAGCGACUGGACCUU
CCUGCACUGCCUGCCCCGCAAGCCCGAGGAGGUGGACGACGAGGUGUUCUACAGC
CCCCGCAGCCUGGUGUUCCCCGAGGCCGAGAACCGCAAGUGGACCAUCAUGGCCG
UGAUGGUGAGCCUGCUGACCGACUACAGCCCCCAGCUGCAGAAGCCCAAGUUCUG
AGGUCUCUAGUAAUGAGCUGGAGCCUCGGUAGCCGUUCCUCCUGCCCGCUGGGCC
UCCCAACGGGCCCUCCUCCCCUCCUUGCACCGGCCCUUCCUGGUCUUUGAAUAAA
GUCUGAGUGGGCAUCUAG
>mARM1920 (SEQ ID NO: 118)
AUUAUUACAUCAAAACAAAAAGCCGCCACCAUGGGAGUAUUCAACCUGCGCAUC
CUGCUGAACAACGCCGCCUUCCGCAACGGCCACAACUUCAUGGUGCGCAACUUCC
GCUGCGGCCAGCCCCUGCAGAACCGGGUGCAGCUGAAGGGCCGCGACCUGCUGAC
CCUGAAGAACUUCACCGGCGAGGAGAUCCGGUACAUGCUGUGGCUGAGCGCCGA
CCUGAAGUUCCGCAUCAAGCAGAAGGGCGAGUACCUGCCCCUGCUGCAGGGCAAG
AGCCUGGGCAUGAUCUUCGAGAAGCGCAGCACCCGCACCCGCCUGAGCACCGAGA
CAGGCUUCGCCCUGCUGGGCGGCCACCCCUGCUUCCUGACCACCCAGGACAUCCA
CCUGGGCGUGAACGAGAGCCUGACCGACACCGCCCGCGUGCUGAGCAGCAUGGCC
GACGCCGUGCUGGCCCGCGUGUACAAGCAGAGCGACCUGGACACCCUGGCCAAGG
AGGCCAGCAUCCCCAUCAUCAACGGCCUGAGCGACCUGUACCACCCCAUCCAGAU
CCUGGCCGACUACCUGACCCUGCAGGAGCACUACAGCAGCCUGAAGGGCCUGACC
CUGAGCUGGAUCGGCGACGGCAACAACAUCCUGCACAGCAUCAUGAUGAGCGCCG
CCAAGUUCGGCAUGCACCUGCAGGCCGCCACCCCCAAGGGCUACGAGCCCGACGC
CAGCGUGACCAAGCUGGCCGAGCAGUACGCCAAGGAGAACGGCACCAAGCUGCUG
CUGACCAACGACCCCCUGGAGGCCGCCCACGGCGGCAACGUGCUGAUCACCGACA
CCUGGAUCAGCAUGGGCCAGGAGGAGGAGAAGAAGAAGCGCCUGCAGGCCUUCC
AGGGCUACCAGGUGACCAUGAAGACCGCCAAGGUGGCCGCCAGCGACUGGACCUU
CCUGCACUGCCUGCCCCGCAAGCCCGAGGAGGUGGACGACGAGGUGUUCUACAGC
CCCCGCAGCCUGGUGUUCCCCGAGGCCGAGAACCGCAAGUGGACCAUCAUGGCCG
UGAUGGUGAGCCUGCUGACCGACUACAGCCCCCAGCUGCAGAAGCCCAAGUUCUG
AGGUCUCUAGUAAUGAGCUGGAGCCUCGGUAGCCGUUCCUCCUGCCCGCUGGGCC
UCCCAACGGGCCCUCCUCCCCUCCUUGCACCGGCCCUUCCUGGUCUUUGAAUAAA
GUCUGAGUGGGCAUCUAG
>mARM1921 (SEQ ID NO: 119)
AUUAUUACAUCAAAACAAAAAGCCGCCACCAUGCUGGUAUUCAACCUGCGCAUCC
UGCUGAACAACGCCGCCUUCCGCAACGGCCACAACUUCAUGGUGCGCAACUUCCG
CUGCGGCCAGCCCCUGCAGAACCGGGUGCAGCUGAAGGGCCGCGACCUGCUGACC
CUGAAGAACUUCACCGGCGAGGAGAUCCGGUACAUGCUGUGGCUGAGCGCCGAC
CUGAAGUUCCGCAUCAAGCAGAAGGGCGAGUACCUGCCCCUGCUGCAGGGCAAG
AGCCUGGGCAUGAUCUUCGAGAAGCGCAGCACCCGCACCCGCCUGAGCACCGAGA
CAGGCUUCGCCCUGCUGGGCGGCCACCCCUGCUUCCUGACCACCCAGGACAUCCA
CCUGGGCGUGAACGAGAGCCUGACCGACACCGCCCGCGUGCUGAGCAGCAUGGCC
GACGCCGUGCUGGCCCGCGUGUACAAGCAGAGCGACCUGGACACCCUGGCCAAGG
AGGCCAGCAUCCCCAUCAUCAACGGCCUGAGCGACCUGUACCACCCCAUCCAGAU
CCUGGCCGACUACCUGACCCUGCAGGAGCACUACAGCAGCCUGAAGGGCCUGACC
CUGAGCUGGAUCGGCGACGGCAACAACAUCCUGCACAGCAUCAUGAUGAGCGCCG
CCAAGUUCGGCAUGCACCUGCAGGCCGCCACCCCCAAGGGCUACGAGCCCGACGC
CAGCGUGACCAAGCUGGCCGAGCAGUACGCCAAGGAGAACGGCACCAAGCUGCUG
CUGACCAACGACCCCCUGGAGGCCGCCCACGGCGGCAACGUGCUGAUCACCGACA
CCUGGAUCAGCAUGGGCCAGGAGGAGGAGAAGAAGAAGCGCCUGCAGGCCUUCC
AGGGCUACCAGGUGACCAUGAAGACCGCCAAGGUGGCCGCCAGCGACUGGACCUU
CCUGCACUGCCUGCCCCGCAAGCCCGAGGAGGUGGACGACGAGGUGUUCUACAGC
CCCCGCAGCCUGGUGUUCCCCGAGGCCGAGAACCGCAAGUGGACCAUCAUGGCCG
UGAUGGUGAGCCUGCUGACCGACUACAGCCCCCAGCUGCAGAAGCCCAAGUUCUG
AGGUCUCUAGUAAUGAGCUGGAGCCUCGGUAGCCGUUCCUCCUGCCCGCUGGGCC
UCCCAACGGGCCCUCCUCCCCUCCUUGCACCGGCCCUUCCUGGUCUUUGAAUAAA
GUCUGAGUGGGCAGCAUCUAG
>mARM1925 (SEQ ID NO: 120)
UCAACACAACAUAUACAAAACAAACGAAUCUCAAGCAAUCAAGCAUUCUACUUC
UAUUGCAGCAAUUUAAAUCAUUUCUUUUAAAGCAAAAGCAAUUUUCUGAAAAUU
UUCACCAUUUACGAACGAUAGCCACCAUGUUGUUCAACUUGAGGAUCUUGUUGA
ACAACGCCGCCUUCAGGAACGGACACAACUUCAUGGUAAGGAACUUCAGGUGCG
GACAGCCCUUGCAGAACAAAGUACAGUUGAAAGGAAGGGACUUGUUGACAUUGA
AAAACUUCACAGGAGAAGAAAUCAAAUACAUGUUGUGGUUGUCGGCCGACUUGA
AAUUCAGGAUCAAACAGAAAGGAGAAUACUUGCCCUUGUUGCAGGGAAAAUCGU
UGGGAAUGAUCUUCGAAAAAAGGUCGACAAGGACAAGGUUGUCGACAGAAACAG
GAUUCGCCUUGUUGGGAGGACACCCCUGCUUCUUGACAACACAGGACAUCCACUU
GGGAGUAAACGAAUCGUUGACAGACACAGCCAGGGUAUUGUCGUCGAUGGCCGA
CGCCGUAUUGGCCAGGGUAUACAAACAGUCGGACUUGGACACAUUGGCCAAAGA
AGCCUCGAUCCCCAUCAUCAACGGAUUGUCGGACUUGUACCACCCCAUCCAGAUC
UUGGCCGACUACUUGACAUUGCAGGAACACUACUCGUCGUUGAAAGGAUUGACA
UUGUCGUGGAUCGGAGACGGAAACAACAUCUUGCACUCGAUCAUGAUGUCGGCC
GCCAAAUUCGGAAUGCACUUGCAGGCCGCCACACCCAAAGGAUACGAACCCGACG
CCUCGGUAACAAAAUUGGCCGAACAGUACGCCAAAGAAAACGGAACAAAAUUGU
UGUUGACAAACGACCCCUUGGAAGCCGCCCACGGAGGAAACGUAUUGAUCACAG
ACACAUGGAUCUCGAUGGGACAGGAAGAAGAAAAAAAAAAAAGGUUGCAGGCCU
UCCAGGGAUACCAGGUAACAAUGAAAACAGCCAAAGUAGCCGCCUCGGACUGGA
CAUUCUUGCACUGCUUGCCCAGGAAACCCGAAGAAGUAGACGACGAAGUAUUCU
ACUCGCCCAGGUCGUUGGUAUUCCCCGAAGCCGAAAACAGGAAAUGGACAAUCA
UGGCCGUAAUGGUAUCGUUGUUGACAGACUACUCGCCCCAGUUGCAGAAACCCA
AAUUCUGAAUAGUGAACUCGAGCUAGUGACUGACUAGGAUCUGGUUACCACUAA
ACCAGCCUCAAGAACACCCGAAUGGAGUCUCUAAGCUACAUAAUACCAACUUACA
CUUACAAAAUGUUGUCCCCCAAAAUGUAGCCAUUCGUAUCUGCUCCUAAUAAAA
AGAAAGUUUCUUCACAUUCUAG
>mARM1926 (SEQ ID NO: 121)
AUUAUUACAUCAAAACAAAAAGCCGCCACCAUGCUGUUCAACCUGCGCAUCCUGC
UGAACAACGCCGCCUUCCGCAACGGCCACAACUUCAUGGUGCGCAACUUCCGCUG
CGGCCAGCCCCUGCAGGGCAAGGUGCAGCUGAAGGGCCGCGACCUGCUGACCCUG
AAGAACUUCACCGGCGAGGAGAUCAAGUACAUGCUGUGGCUGAGCGCCGACCUG
AAGUUCCGCAUCAAGCAGAAGGGCGAGUACCUGCCCCUGCUGCAGGGCAAGAGCC
UGGGCAUGAUCUUCGAGAAGCGCAGCACCCGCACCCGCCUGAGCACCGAGACAGG
CUUCGCCCUGCUGGGCGGCCACCCCUGCUUCCUGACCACCCAGGACAUCCACCUG
GGCGUGAACGAGAGCCUGACCGACACCGCCCGCGUGCUGAGCAGCAUGGCCGACG
CCGUGCUGGCCCGCGUGUACAAGCAGAGCGACCUGGACACCCUGGCCAAGGAGGC
CAGCAUCCCCAUCAUCAACGGCCUGAGCGACCUGUACCACCCCAUCCAGAUCCUG
GCCGACUACCUGACCCUGCAGGAGCACUACAGCAGCCUGAAGGGCCUGACCCUGA
GCUGGAUCGGCGACGGCAACAACAUCCUGCACAGCAUCAUGAUGAGCGCCGCCAA
GUUCGGCAUGCACCUGCAGGCCGCCACCCCCAAGGGCUACGAGCCCGACGCCAGC
GUGACCAAGCUGGCCGAGCAGUACGCCAAGGAGAACGGCACCAAGCUGCUGCUG
ACCAACGACCCCCUGGAGGCCGCCCACGGCGGCAACGUGCUGAUCACCGACACCU
GGAUCAGCAUGGGCCAGGAGGAGGAGAAGAAGAAGCGCCUGCAGGCCUUCCAGG
GCUACCAGGUGACCAUGAAGACCGCCAAGGUGGCCGCCAGCGACUGGACCUUCCU
GCACUGCCUGCCCCGCAAGCCCGAGGAGGUGGACGACGAGGUGUUCUACAGCCCC
CGCAGCCUGGUGUUCCCCGAGGCCGAGAACCGCAAGUGGACCAUCAUGGCCGUGA
UGGUGAGCCUGCUGACCGACUACAGCCCCCAGCUGCAGAAGCCCAAGUUCUGAGG
UCUCUAGUAAUGAGCUGGAGCCUCGGUAGCCGUUCCUCCUGCCCGCUGGGCCUCC
CAACGGGCCCUCCUCCCCUCCUUGCACCGGCCCUUCCUGGUCUUUGAAUAAAGUC
UGAGUGGGCAUCUAG
>mARM1927 (SEQ ID NO: 122)
AUUAUUACAUCAAAACAAAAAGCCGCCACCAUGCUGUUCAACCUGCGCAUCCUGC
UGAACAACGCCGCCUUCCGCAACGGCCACAACUUCAUGGUGCGCAACUUCCGCUG
CGGCCAGCCCCUGCAGGGCCGGGUGCAGCUGAAGGGCCGCGACCUGCUGACCCUG
AAGAACUUCACCGGCGAGGAGAUCAAGUACAUGCUGUGGCUGAGCGCCGACCUG
AAGUUCCGCAUCAAGCAGAAGGGCGAGUACCUGCCCCUGCUGCAGGGCAAGAGCC
UGGGCAUGAUCUUCGAGAAGCGCAGCACCCGCACCCGCCUGAGCACCGAGACAGG
CUUCGCCCUGCUGGGCGGCCACCCCUGCUUCCUGACCACCCAGGACAUCCACCUG
GGCGUGAACGAGAGCCUGACCGACACCGCCCGCGUGCUGAGCAGCAUGGCCGACG
CCGUGCUGGCCCGCGUGUACAAGCAGAGCGACCUGGACACCCUGGCCAAGGAGGC
CAGCAUCCCCAUCAUCAACGGCCUGAGCGACCUGUACCACCCCAUCCAGAUCCUG
GCCGACUACCUGACCCUGCAGGAGCACUACAGCAGCCUGAAGGGCCUGACCCUGA
GCUGGAUCGGCGACGGCAACAACAUCCUGCACAGCAUCAUGAUGAGCGCCGCCAA
GUUCGGCAUGCACCUGCAGGCCGCCACCCCCAAGGGCUACGAGCCCGACGCCAGC
GUGACCAAGCUGGCCGAGCAGUACGCCAAGGAGAACGGCACCAAGCUGCUGCUG
ACCAACGACCCCCUGGAGGCCGCCCACGGCGGCAACGUGCUGAUCACCGACACCU
GGAUCAGCAUGGGCCAGGAGGAGGAGAAGAAGAAGCGCCUGCAGGCCUUCCAGG
GCUACCAGGUGACCAUGAAGACCGCCAAGGUGGCCGCCAGCGACUGGACCUUCCU
GCACUGCCUGCCCCGCAAGCCCGAGGAGGUGGACGACGAGGUGUUCUACAGCCCC
CGCAGCCUGGUGUUCCCCGAGGCCGAGAACCGCAAGUGGACCAUCAUGGCCGUGA
UGGUGAGCCUGCUGACCGACUACAGCCCCCAGCUGCAGAAGCCCAAGUUCUGAGG
UCUCUAGUAAUGAGCUGGAGCCUCGGUAGCCGUUCCUCCUGCCCGCUGGGCCUCC
CAACGGGCCCUCCUCCCCUCCUUGCACCGGCCCUUCCUGGUCUUUGAAUAAAGUC
UGAGUGGGCAUCUAG
>mARM1928 (SEQ ID NO: 123)
AUUAUUACAUCAAAACAAAAAGCCGCCACCAUGCUGUUCAACCUGCGCAUCCUGC
UGAACAACGCCGCCUUCCGCAACGGCCACAACUUCAUGGUGCGCAACUUCCGCUG
CGGCCAGCCCCUGCAGGGCCGGGUGCAGCUGAAGGGCCGCGACCUGCUGACCCUG
AAGAACUUCACCGGCGAGGAGAUCCGGUACAUGCUGUGGCUGAGCGCCGACCUG
AAGUUCCGCAUCAAGCAGAAGGGCGAGUACCUGCCCCUGCUGCAGGGCAAGAGCC
UGGGCAUGAUCUUCGAGAAGCGCAGCACCCGCACCCGCCUGAGCACCGAGACAGG
CUUCGCCCUGCUGGGCGGCCACCCCUGCUUCCUGACCACCCAGGACAUCCACCUG
GGCGUGAACGAGAGCCUGACCGACACCGCCCGCGUGCUGAGCAGCAUGGCCGACG
CCGUGCUGGCCCGCGUGUACAAGCAGAGCGACCUGGACACCCUGGCCAAGGAGGC
CAGCAUCCCCAUCAUCAACGGCCUGAGCGACCUGUACCACCCCAUCCAGAUCCUG
GCCGACUACCUGACCCUGCAGGAGCACUACAGCAGCCUGAAGGGCCUGACCCUGA
GCUGGAUCGGCGACGGCAACAACAUCCUGCACAGCAUCAUGAUGAGCGCCGCCAA
GUUCGGCAUGCACCUGCAGGCCGCCACCCCCAAGGGCUACGAGCCCGACGCCAGC
GUGACCAAGCUGGCCGAGCAGUACGCCAAGGAGAACGGCACCAAGCUGCUGCUG
ACCAACGACCCCCUGGAGGCCGCCCACGGCGGCAACGUGCUGAUCACCGACACCU
GGAUCAGCAUGGGCCAGGAGGAGGAGAAGAAGAAGCGCCUGCAGGCCUUCCAGG
GCUACCAGGUGACCAUGAAGACCGCCAAGGUGGCCGCCAGCGACUGGACCUUCCU
GCACUGCCUGCCCCGCAAGCCCGAGGAGGUGGACGACGAGGUGUUCUACAGCCCC
CGCAGCCUGGUGUUCCCCGAGGCCGAGAACCGCAAGUGGACCAUCAUGGCCGUGA
UGGUGAGCCUGCUGACCGACUACAGCCCCCAGCUGCAGAAGCCCAAGUUCUGAGG
UCUCUAGUAAUGAGCUGGAGCCUCGGUAGCCGUUCCUCCUGCCCGCUGGGCCUCC
CAACGGGCCCUCCUCCCCUCCUUGCACCGGCCCUUCCUGGUCUUUGAAUAAAGUC
UGAGUGGGCAUCUAG
>mARM1929 (SEQ ID NO: 124)
GGCAGAAAAAUUUGCUACAUUGUUUCACAAACUUCAAAUAUUAUUCAUUUAUUU
AGAUCUAUUAUUACAUCAAAACAAAAAGCCGCCACCAUGCUGUUCAACCUGCGC
AUCCUGCUGAACAACGCCGCCUUCCGCAACGGCCACAACUUCAUGGUGCGCAACU
UCCGCUGCGGCCAGCCCCUGCAGGGCAAGGUGCAGCUGAAGGGCCGCGACCUGCU
GACCCUGAAGAACUUCACCGGCGAGGAGAUCAAGUACAUGCUGUGGCUGAGCGC
CGACCUGAAGUUCCGCAUCAAGCAGAAGGGCGAGUACCUGCCCCUGCUGCAGGGC
AAGAGCCUGGGCAUGAUCUUCGAGAAGCGCAGCACCCGCACCCGCCUGAGCACCG
AGACAGGCUUCGCCCUGCUGGGCGGCCACCCCUGCUUCCUGACCACCCAGGACAU
CCACCUGGGCGUGAACGAGAGCCUGACCGACACCGCCCGCGUGCUGAGCAGCAUG
GCCGACGCCGUGCUGGCCCGCGUGUACAAGCAGAGCGACCUGGACACCCUGGCCA
AGGAGGCCAGCAUCCCCAUCAUCAACGGCCUGAGCGACCUGUACCACCCCAUCCA
GAUCCUGGCCGACUACCUGACCCUGCAGGAGCACUACAGCAGCCUGAAGGGCCUG
ACCCUGAGCUGGAUCGGCGACGGCAACAACAUCCUGCACAGCAUCAUGAUGAGCG
CCGCCAAGUUCGGCAUGCACCUGCAGGCCGCCACCCCCAAGGGCUACGAGCCCGA
CGCCAGCGUGACCAAGCUGGCCGAGCAGUACGCCAAGGAGAACGGCACCAAGCUG
CUGCUGACCAACGACCCCCUGGAGGCCGCCCACGGCGGCAACGUGCUGAUCACCG
ACACCUGGAUCAGCAUGGGCCAGGAGGAGGAGAAGAAGAAGCGCCUGCAGGCCU
UCCAGGGCUACCAGGUGACCAUGAAGACCGCCAAGGUGGCCGCCAGCGACUGGAC
CUUCCUGCACUGCCUGCCCCGCAAGCCCGAGGAGGUGGACGACGAGGUGUUCUAC
AGCCCCCGCAGCCUGGUGUUCCCCGAGGCCGAGAACCGCAAGUGGACCAUCAUGG
CCGUGAUGGUGAGCCUGCUGACCGACUACAGCCCCCAGCUGCAGAAGCCCAAGUU
CUGAGGUCUCUAGUAAUGAGCUGGAGCCUCGGUAGCCGUUCCUCCUGCCCGCUGG
GCCUCCCAACGGGCCCUCCUCCCCUCCUUGCACCGGCCCUUCCUGGUCUUUGAAU
AAAGUCUGAGUGGGCAUCUAG
AT1G67090> (SEQ ID NO: 125)
CACAAAGAGUAAAGAAGAACA
AT1G35720> (SEQ ID NO: 126)
AACACUAAAAGUAGAAGAAAA
AT5G45900> (SEQ ID NO: 127)
CUCAGAAAGAUAAGAUCAGCC
>pARM563 (SEQ ID NO: 128)
ATGCTGTTTAATCTGAGGATCCTGTTAAACAATGCAGCTTTTAGAAATGGTCACAAC
TTCATGGTTCGAAATTTTCGGTGTGGACAACCACTACAAAATAAAGTGCAGCTGAA
GGGCCGTGACCTTCTCACTCTAAAAAACTTTACCGGAGAAGAAATTAAATATATGC
TATGGCTATCAGCAGATCTGAAATTTAGGATAAAACAGAAAGGAGAGTATTTGCCT
TTATTGCAAGGGAAGTCCTTAGGCATGATTTTTGAGAAAAGAAGTACTCGAACAAG
ATTGTCTACAGAAACAGGCTTTGCACTTCTGGGAGGACATCCTTGTTTTCTTACCAC
ACAAGATATTCATTTGGGTGTGAATGAAAGTCTCACGGACACGGCCCGTGTATTGTC
TAGCATGGCAGATGCAGTATTGGCTCGAGTGTATAAACAATCAGATTTGGACACCC
TGGCTAAAGAAGCATCCATCCCAATTATCAATGGGCTGTCAGATTTGTACCATCCTA
TCCAGATCCTGGCTGATTACCTCACGCTCCAGGAACACTATAGCTCTCTGAAAGGTC
TTACCCTCAGCTGGATCGGGGATGGGAACAATATCCTGCACTCCATCATGATGAGC
GCAGCGAAATTCGGAATGCACCTTCAGGCAGCTACTCCAAAGGGTTATGAGCCGGA
TGCTAGTGTAACCAAGTTGGCAGAGCAGTATGCCAAAGAGAATGGTACCAAGCTGT
TGCTGACAAATGATCCATTGGAAGCAGCGCATGGAGGCAATGTATTAATTACAGAC
ACTTGGATAAGCATGGGACAAGAAGAGGAGAAGAAAAAGCGGCTCCAGGCTTTCC
AAGGTTACCAGGTTACAATGAAGACTGCTAAAGTTGCTGCCTCTGACTGGACATTTT
TACACTGCTTGCCCAGAAAGCCAGAAGAAGTGGATGATGAAGTCTTTTATTCTCCTC
GATCACTAGTGTTCCCAGAGGCAGAAAACAGAAAGTGGACAATCATGGCTGTCATG
GTGTCCCTGCTGACAGATTACTCACCTCAGCTCCAGAAGCCTAAATTTTGA
>pARM564 (SEQ ID NO: 129)
ATGCTCTTTAATCTGCGCATCTTACTGAACAACGCCGCATTCCGGAACGGTCACAAC
TTCATGGTCCGCAATTTCCGCTGTGGCCAGCCGCTTCAAAACAAGGTCCAGCTGAAG
GGACGGGATCTGCTGACACTGAAGAACTTCACCGGAGAAGAGATCAAGTACATGCT
GTGGCTCAGCGCAGACTTGAAGTTCCGGATCAAGCAGAAGGGAGAATACTTGCCCC
TGCTGCAAGGAAAGTCGCTGGGAATGATTTTTGAGAAGCGGTCAACTCGCACCAGA
CTCTCCACCGAAACTGGTTTCGCACTGCTTGGCGGGCACCCTTGCTTCCTGACGACT
CAGGACATCCACCTCGGCGTGAACGAATCGCTAACCGATACCGCCAGAGTGCTTTC
TTCCATGGCCGACGCGGTGCTGGCCAGGGTGTACAAGCAGTCCGACCTCGATACCT
TGGCAAAGGAGGCTTCCATTCCCATCATCAACGGCCTGAGCGACCTGTACCACCCA
ATCCAAATCCTGGCTGACTACCTGACCCTGCAAGAGCACTACAGCAGCCTGAAGGG
TCTGACCCTGTCATGGATTGGCGATGGAAACAATATTCTGCACTCCATCATGATGTC
CGCCGCGAAGTTCGGAATGCATCTGCAAGCCGCCACTCCAAAAGGATACGAACCGG
ATGCGTCCGTGACCAAGTTGGCGGAACAGTACGCGAAGGAGAACGGAACCAAGCT
TCTGCTGACTAACGACCCCCTCGAGGCTGCGCATGGGGGCAACGTGCTGATTACCG
ACACCTGGATCTCCATGGGGCAGGAGGAAGAGAAGAAGAAGAGACTGCAGGCATT
CCAGGGGTACCAGGTCACCATGAAAACCGCAAAAGTGGCAGCTTCGGACTGGACTT
TCCTGCATTGCCTGCCGAGGAAGCCGGAGGAAGTCGACGACGAAGTGTTCTACTCG
CCTCGGTCCCTGGTGTTCCCCGAGGCCGAAAACCGGAAGTGGACCATCATGGCCGT
GATGGTGTCCTTGCTGACTGACTATAGCCCGCAGCTGCAGAAGCCTAAGTTCTAG
>pARM565 (SEQ ID NO: 130)
ATGCTGTTTAACCTACGTATTTTGCTCAACAATGCAGCCTTTAGAAACGGACATAAC
TTTATGGTTCGAAACTTTCGCTGCGGGCAGCCACTGCAGAACAAGGTCCAGCTGAA
AGGGAGAGATTTGCTCACGCTGAAGAACTTTACTGGCGAAGAAATCAAGTATATGC
TGTGGTTGTCCGCGGACCTCAAGTTTCGGATTAAGCAGAAAGGGGAGTATCTGCCA
CTGCTGCAAGGAAAGAGCCTCGGCATGATCTTCGAGAAGCGGAGCACTCGGACCAG
GCTGAGTACCGAAACTGGCTTCGCATTGTTGGGTGGACATCCATGTTTTCTGACAAC
GCAGGACATTCATCTGGGCGTGAACGAGAGTCTGACGGACACAGCTCGCGTTCTGT
CCTCTATGGCTGATGCGGTGTTGGCCCGGGTCTATAAGCAGTCCGATTTGGACACCT
TGGCTAAGGAAGCTAGCATACCGATTATCAATGGGCTGTCCGACCTGTATCACCCTA
TTCAAATCCTGGCCGACTACCTCACACTGCAAGAACACTATAGCTCATTGAAGGGA
CTGACCCTGAGCTGGATAGGGGACGGAAACAACATCCTACATAGCATTATGATGTC
CGCTGCCAAGTTTGGCATGCATCTTCAAGCCGCCACGCCAAAGGGTTATGAGCCCG
ACGCGTCAGTGACAAAGCTGGCCGAGCAGTACGCTAAGGAGAATGGTACCAAATTA
CTGCTGACTAATGATCCACTGGAGGCTGCACATGGCGGCAATGTACTGATCACCGA
CACATGGATCTCGATGGGCCAGGAGGAAGAAAAGAAGAAGAGGCTTCAGGCCTTC
CAAGGCTACCAGGTCACCATGAAAACAGCTAAGGTTGCAGCATCTGATTGGACCTT
TCTGCACTGTCTGCCAAGGAAGCCCGAAGAGGTGGACGATGAAGTATTCTATAGCC
CACGGAGTTTGGTGTTCCCTGAGGCTGAAAATAGGAAGTGGACAATTATGGCCGTA
ATGGTGTCCCTGTTAACCGACTACTCTCCGCAACTGCAGAAACCTAAGTTTTAG
>pARM566 (SEQ ID NO: 131)
ATGCTGTTTAACTTAAGGATCCTGCTGAACAACGCCGCTTTTCGTAACGGTCATAAC
TTTATGGTCCGGAACTTTAGATGTGGCCAGCCGCTGCAGAACAAGGTTCAGCTGAA
GGGGAGGGATCTGCTGACCTTGAAGAACTTTACCGGCGAAGAGATCAAGTACATGT
TGTGGCTGAGCGCCGATCTGAAGTTTAGGATTAAGCAGAAGGGGGAGTATTTGCCA
CTGCTGCAAGGAAAATCCTTGGGGATGATCTTCGAGAAGCGCTCCACTAGAACCCG
GCTAAGCACAGAAACCGGCTTCGCACTTCTGGGTGGACATCCCTGTTTTCTGACGAC
GCAGGATATACACCTGGGCGTGAATGAGAGTCTGACGGACACAGCTAGGGTGTTGA
GCAGCATGGCCGATGCAGTACTGGCCCGCGTTTATAAGCAGAGCGACTTGGACACA
CTGGCCAAGGAAGCGTCAATTCCGATTATCAATGGGCTGTCAGACCTGTATCATCCC
ATTCAAATCTTGGCTGACTATCTGACCCTGCAAGAACATTACAGCTCCCTGAAGGGC
CTCACGTTGTCCTGGATTGGCGACGGAAACAACATTCTGCATTCGATCATGATGAGC
GCTGCTAAGTTTGGCATGCACCTCCAAGCCGCTACACCTAAGGGATATGAGCCTGA
TGCCAGCGTAACCAAGCTGGCCGAACAGTACGCGAAGGAGAATGGCACGAAACTG
CTGTTGACAAATGACCCACTGGAGGCAGCTCACGGTGGCAACGTGCTGATCACCGA
CACGTGGATATCTATGGGACAGGAAGAAGAGAAGAAGAAGCGGCTGCAGGCATTC
CAAGGGTATCAGGTCACCATGAAAACGGCCAAGGTTGCTGCATCCGACTGGACATT
TCTGCATTGCTTGCCCCGCAAACCAGAAGAAGTAGACGACGAAGTCTTTTATTCCCC
ACGGTCGCTGGTGTTCCCCGAGGCGGAGAATCGAAAGTGGACGATTATGGCCGTGA
TGGTGTCCCTGCTGACTGATTACTCTCCCCAACTGCAAAAGCCTAAGTTTTAG
>pARM567 (SEQ ID NO: 132)
ATGCTTTTCAACCTGAGGATCCTCCTGAACAACGCCGCCTTTCGCAATGGTCACAAC
TTTATGGTCCGGAACTTCAGATGCGGCCAGCCGCTGCAGAACAAGGTCCAGCTGAA
GGGACGGGATCTGCTGACTCTGAAGAACTTCACCGGAGAAGAGATCAAGTACATGC
TGTGGCTGTCGGCCGACCTGAAGTTCAGGATCAAGCAGAAGGGAGAATACCTCCCG
CTGCTGCAAGGAAAGTCCCTGGGCATGATTTTCGAGAAGCGCTCGACCAGAACTCG
GTTGTCCACCGAAACCGGGTTTGCGCTGCTGGGCGGACATCCTTGCTTCCTGACGAC
TCAGGATATTCACCTGGGAGTGAACGAGTCGCTGACCGACACCGCCAGAGTGCTGA
GCTCGATGGCCGACGCCGTGTTGGCACGCGTGTACAAGCAGTCCGATCTGGATACC
CTGGCCAAAGAAGCTTCCATCCCGATCATTAACGGGCTGAGCGACCTCTACCACCC
CATTCAAATCCTGGCCGACTACCTGACTCTGCAAGAACACTACAGCTCGCTGAAGG
GGTTGACTCTGTCCTGGATCGGCGACGGAAACAACATCCTGCACTCCATCATGATGT
CGGCCGCAAAGTTCGGCATGCATTTGCAAGCCGCCACCCCAAAGGGCTACGAACCA
GACGCGAGCGTCACCAAGCTGGCCGAACAGTACGCGAAGGAAAATGGTACTAAGC
TGCTGCTGACCAACGACCCATTGGAAGCTGCCCATGGTGGAAACGTGCTGATCACC
GACACCTGGATCTCGATGGGCCAGGAAGAGGAGAAGAAGAAGCGGCTGCAGGCGT
TCCAGGGGTATCAGGTCACCATGAAAACAGCCAAAGTGGCAGCGTCAGACTGGACC
TTCCTCCACTGTCTGCCTCGCAAGCCAGAGGAGGTGGACGACGAGGTGTTCTACTCC
CCTCGGTCCCTCGTGTTCCCTGAGGCTGAGAACCGGAAGTGGACCATTATGGCCGTG
ATGGTGTCACTCCTGACTGATTACTCCCCGCAACTGCAGAAGCCCAAGTTCTAG
>pARM568 (SEQ ID NO: 133)
ATGCTGTTTAACCTGAGGATCCTATTGAACAATGCTGCTTTTCGTAATGGCCATAAC
TTTATGGTTCGGAACTTTAGATGCGGGCAGCCACTGCAGAACAAGGTCCAGTTGAA
AGGCCGCGATCTGTTGACATTGAAGAACTTTACCGGCGAAGAGATTAAGTATATGC
TGTGGCTGTCTGCTGACCTCAAGTTTCGAATCAAGCAGAAGGGCGAATATCTCCCCC
TGCTGCAAGGAAAGTCTCTCGGCATGATCTTTGAGAAGCGGAGTACCCGAACACGG
CTGAGCACCGAAACGGGCTTCGCACTGCTGGGGGGCCATCCCTGTTTTCTGACAAC
GCAGGACATCCACTTGGGGGTTAACGAATCATTGACTGATACCGCCCGCGTACTGT
CATCCATGGCCGACGCTGTGCTGGCTAGGGTGTACAAGCAGTCAGATCTGGATACA
CTGGCCAAGGAAGCTAGCATACCAATCATCAATGGACTGAGTGACCTTTATCACCC
GATTCAAATACTAGCCGATTATCTGACCCTGCAAGAGCATTACTCCTCGCTGAAAGG
CCTCACGCTGTCCTGGATCGGCGACGGCAACAACATTCTGCATAGTATTATGATGTC
TGCTGCCAAATTCGGCATGCATCTGCAAGCTGCTACGCCGAAGGGTTATGAACCCG
ACGCGTCAGTTACGAAGCTCGCTGAGCAGTATGCAAAGGAGAATGGCACAAAGCTG
TTGCTTACCAACGATCCCCTGGAAGCTGCTCATGGCGGCAATGTGCTGATTACTGAC
ACCTGGATTTCAATGGGCCAGGAGGAGGAGAAGAAGAAGAGGTTACAGGCTTTTCA
AGGTTACCAAGTCACGATGAAAACCGCTAAGGTCGCAGCCAGCGACTGGACATTCC
TGCACTGTCTGCCAAGAAAGCCGGAAGAAGTGGACGACGAGGTGTTCTATTCCCCG
CGGTCTTTGGTGTTTCCGGAGGCCGAAAACAGGAAATGGACCATTATGGCCGTGAT
GGTATCGTTGCTGACGGACTACAGCCCTCAGTTGCAAAAGCCCAAGTTCTAG
>pARM569 (SEQ ID NO: 134)
ATGCTCTTTAACCTCCGCATCCTCCTCAACAACGCCGCCTTCCGGAATGGGCATAAC
TTCATGGTCCGGAACTTCAGATGCGGCCAGCCCCTGCAAAACAAGGTCCAGTTGAA
GGGACGGGACCTCCTTACGCTGAAGAACTTTACCGGAGAAGAGATTAAGTACATGC
TGTGGTTGTCCGCTGACCTCAAGTTCCGCATTAAGCAGAAGGGAGAATATCTGCCG
CTGCTGCAAGGAAAGAGCCTGGGCATGATCTTCGAAAAGCGCTCCACTAGAACCCG
GCTGTCGACTGAGACTGGATTCGCCTTGCTCGGTGGACACCCGTGCTTCCTGACGAC
CCAGGACATCCACCTGGGAGTGAACGAGTCACTTACGGATACCGCGAGGGTGCTGT
CCTCAATGGCCGACGCAGTGCTCGCGCGCGTGTACAAGCAGTCAGATCTGGATACC
CTGGCCAAGGAAGCCAGCATTCCCATCATCAACGGACTGAGCGACCTTTACCACCC
AATCCAGATCCTCGCCGACTACTTAACCCTGCAAGAGCACTACAGCTCCCTGAAGG
GACTGACTCTGTCCTGGATCGGGGATGGAAACAACATCCTGCACTCCATCATGATGT
CTGCCGCTAAGTTTGGGATGCATCTGCAAGCCGCAACCCCTAAGGGATACGAGCCC
GACGCCTCGGTGACCAAACTTGCGGAACAGTACGCCAAGGAAAACGGTACCAAGCT
GCTGCTGACCAACGACCCTCTGGAAGCGGCCCACGGAGGAAATGTGCTGATTACCG
ACACCTGGATTTCGATGGGCCAGGAGGAGGAGAAGAAGAAGAGACTGCAGGCGTT
CCAGGGATATCAGGTCACCATGAAAACCGCCAAGGTCGCCGCCAGCGACTGGACCT
TCCTGCACTGTCTCCCTCGGAAACCGGAAGAAGTGGATGACGAGGTGTTCTACTCCC
CGCGCTCGCTGGTGTTCCCGGAGGCTGAAAACAGGAAGTGGACAATCATGGCCGTG
ATGGTGTCCCTGTTGACCGACTACTCCCCACAACTGCAGAAGCCCAAGTTCTAG
>pARM570 (SEQ ID NO: 135)
ATGCTTTTCAATCTGCGCATCCTCCTGAACAACGCCGCCTTCCGCAATGGACACAAC
TTTATGGTCCGCAACTTCCGCTGTGGGCAGCCGCTGCAGAACAAGGTCCAGCTCAA
GGGGAGAGATCTCCTGACCCTGAAGAACTTCACTGGAGAGGAGATCAAGTACATGC
TGTGGCTGTCCGCCGACCTGAAATTTCGGATTAAGCAGAAGGGCGAATACCTCCCA
CTGCTGCAAGGAAAGTCTTTGGGCATGATCTTCGAAAAGAGAAGCACCCGGACCCG
GTTGAGCACCGAAACTGGGTTCGCGCTCCTCGGTGGACACCCGTGCTTCCTGACCAC
CCAAGATATTCATCTGGGTGTCAACGAAAGCCTGACCGACACCGCCAGGGTGCTGT
CATCCATGGCTGACGCAGTGCTCGCCCGGGTGTACAAGCAGTCAGACCTGGACACC
CTCGCCAAGGAAGCTTCGATCCCTATCATCAACGGACTTTCCGACCTGTACCACCCC
ATCCAAATTCTGGCCGACTACCTGACTCTGCAAGAACACTATAGCTCGCTGAAAGG
ACTTACTCTGTCCTGGATCGGGGACGGCAACAACATTCTCCATTCCATCATGATGTC
CGCTGCCAAGTTCGGAATGCACCTTCAAGCAGCGACTCCCAAGGGATACGAACCTG
ATGCCTCCGTGACTAAGCTGGCAGAGCAGTACGCCAAGGAGAACGGTACAAAGCTG
CTGCTCACGAACGACCCCCTGGAGGCGGCCCACGGCGGAAACGTGCTGATTACCGA
TACCTGGATCTCAATGGGCCAGGAAGAGGAGAAGAAGAAGCGGCTCCAGGCGTTTC
AAGGCTACCAGGTCACCATGAAAACCGCGAAGGTCGCCGCCTCCGACTGGACTTTC
TTGCACTGCCTGCCGCGGAAGCCCGAGGAAGTGGATGACGAAGTGTTCTACTCGCC
GAGATCGTTGGTGTTCCCTGAGGCCGAAAACAGGAAGTGGACCATCATGGCCGTGA
TGGTGTCCCTGCTGACTGATTACAGCCCACAGCTGCAGAAGCCTAAGTTCTAG
>pARM571 (SEQ ID NO: 136)
ATGCTTTTCAATCTCCGCATCCTCCTTAACAACGCCGCGTTTAGAAACGGCCACAAC
TTCATGGTCCGGAACTTCAGATGTGGCCAGCCGCTTCAAAACAAGGTCCAGCTGAA
GGGCCGGGATCTTCTGACCCTGAAGAACTTTACTGGCGAAGAGATCAAGTACATGC
TCTGGCTCTCCGCGGACTTGAAGTTCCGCATTAAGCAGAAGGGGGAATACCTTCCG
CTGCTTCAAGGAAAGAGCCTCGGCATGATCTTTGAGAAGCGCTCAACCAGGACCCG
CCTTTCTACTGAAACTGGGTTCGCGCTGCTCGGTGGCCACCCCTGCTTCCTGACGAC
CCAGGACATCCACCTCGGAGTGAACGAATCCCTCACCGATACCGCCCGGGTGTTAT
CGAGCATGGCAGATGCCGTGCTGGCCAGGGTGTACAAACAGTCCGATCTGGACACT
CTGGCCAAGGAGGCGTCAATTCCTATTATCAACGGCCTTAGTGACCTCTACCATCCG
ATTCAGATCCTGGCCGATTACCTCACCCTGCAAGAACACTACAGCTCCCTGAAGGGT
CTGACATTGTCCTGGATCGGCGACGGCAACAACATTCTCCATTCCATCATGATGTCC
GCCGCAAAATTCGGCATGCATCTTCAAGCCGCCACGCCGAAGGGTTACGAGCCCGA
CGCTTCCGTGACTAAGCTCGCCGAGCAGTACGCTAAGGAGAACGGAACCAAGCTTC
TGCTGACTAACGACCCACTAGAAGCAGCCCACGGGGGCAACGTGCTTATTACTGAC
ACCTGGATCTCCATGGGCCAGGAAGAAGAGAAAAAGAAGCGGCTGCAGGCGTTCC
AGGGATATCAGGTCACCATGAAAACCGCCAAGGTCGCTGCCTCCGACTGGACCTTC
CTGCACTGCCTGCCTCGCAAGCCTGAAGAAGTGGACGACGAGGTGTTCTACTCGCC
ACGGAGCCTCGTGTTCCCCGAGGCCGAGAATAGAAAGTGGACCATCATGGCCGTGA
TGGTGTCACTGCTCACCGACTACAGCCCGCAGCTTCAGAAGCCCAAGTTCTAG
>pARM572 (SEQ ID NO: 137)
ATGCTTTTCAACCTGAGAATCCTCCTGAACAACGCCGCCTTCCGCAATGGTCATAAC
TTCATGGTCCGCAACTTTCGCTGCGGACAGCCTCTCCAAAACAAGGTCCAGCTCAAG
GGGCGCGACCTCCTCACACTGAAGAACTTCACTGGAGAAGAAATCAAGTACATGCT
GTGGCTGAGCGCCGATCTGAAGTTCCGGATCAAGCAGAAGGGAGAGTACCTTCCTC
TGCTGCAAGGGAAGTCCTTGGGAATGATTTTCGAGAAGCGGTCCACCCGGACCAGG
CTGAGCACTGAAACTGGCTTCGCCCTGCTGGGAGGCCACCCTTGTTTCCTGACCACT
CAGGACATCCACCTGGGCGTGAACGAGTCCCTGACCGATACTGCCAGAGTGCTGTC
CTCCATGGCCGACGCCGTGCTCGCCCGGGTGTACAAGCAGTCAGACCTCGATACGC
TGGCCAAGGAAGCCTCCATTCCCATTATCAATGGTCTGTCGGACCTCTACCATCCAA
TCCAAATCCTCGCCGACTACCTGACTCTGCAAGAACACTACAGCTCACTCAAGGGC
CTCACCCTCTCCTGGATCGGCGACGGAAACAACATCCTTCACTCGATTATGATGTCG
GCCGCGAAGTTCGGGATGCACCTCCAAGCTGCCACTCCAAAAGGCTACGAGCCGGA
TGCCTCAGTGACTAAGTTGGCGGAACAGTATGCGAAGGAGAACGGTACCAAGCTCC
TGCTGACTAACGACCCGCTGGAGGCCGCCCACGGGGGAAACGTGCTCATCACCGAT
ACTTGGATTTCCATGGGACAGGAGGAAGAGAAGAAGAAGCGGTTGCAGGCATTTCA
GGGCTACCAGGTCACCATGAAAACTGCCAAAGTCGCCGCCAGCGACTGGACCTTCC
TGCACTGCCTGCCCCGCAAGCCTGAAGAAGTGGACGACGAGGTGTTCTACTCTCCC
CGGTCCCTCGTGTTCCCTGAGGCCGAAAACAGGAAGTGGACCATCATGGCTGTGAT
GGTGTCCCTCCTGACCGACTACAGCCCTCAGCTCCAAAAACCCAAGTTTTAG
>pARM573 (SEQ ID NO: 138)
ATGCTTTTCAACCTGAGAATCCTCTTGAACAATGCTGCTTTTCGGAATGGCCACAAC
TTTATGGTTCGGAACTTCCGTTGCGGCCAGCCTTTACAAAACAAGGTCCAGCTGAAG
GGCCGGGATTTGCTCACACTGAAGAACTTTACTGGGGAGGAGATTAAGTATATGCT
GTGGCTGTCCGCTGACCTGAAGTTTAGGATCAAGCAGAAGGGCGAATATCTGCCGC
TGCTGCAAGGGAAAAGTCTGGGCATGATTTTTGAAAAGCGCTCTACCCGGACCAGA
CTGTCTACGGAAACAGGCTTTGCCCTGCTGGGCGGCCACCCCTGTTTTCTGACAACG
CAGGACATCCATCTGGGCGTGAACGAATCACTGACCGATACTGCTCGGGTACTCAG
TTCTATGGCTGACGCAGTGCTGGCTAGGGTGTACAAGCAGAGCGACTTGGACACAC
TGGCTAAGGAGGCCAGCATCCCCATTATCAATGGCCTGTCTGATTTGTACCATCCCA
TTCAAATCCTGGCTGATTATCTGACACTACAAGAGCATTACTCAAGTCTGAAGGGTT
TGACTCTCTCCTGGATCGGCGACGGCAACAACATTTTACATTCCATTATGATGAGTG
CTGCTAAGTTTGGCATGCATTTGCAAGCTGCTACCCCAAAGGGCTATGAACCTGACG
CTAGCGTAACCAAGTTGGCCGAACAGTATGCTAAAGAGAATGGCACCAAGCTGCTC
CTGACGAATGACCCCCTGGAAGCTGCTCATGGCGGAAACGTACTTATAACTGATAC
ATGGATTAGCATGGGCCAGGAAGAGGAGAAGAAGAAGAGACTGCAGGCCTTCCAA
GGCTATCAGGTCACCATGAAAACTGCCAAGGTTGCAGCTAGCGACTGGACCTTCCT
GCACTGTTTGCCGAGGAAACCCGAGGAGGTGGACGATGAAGTCTTTTATTCTCCCC
GCTCCTTGGTGTTTCCCGAGGCTGAAAATCGAAAGTGGACGATAATGGCAGTGATG
GTGTCCCTACTGACCGACTATTCTCCACAACTGCAGAAGCCTAAATTCTAG
>pARM574 (SEQ ID NO: 139)
ATGCTTTTCAATCTGAGGATCCTGCTGAACAACGCTGCTTTTCGCAACGGTCATAAC
TTTATGGTTCGCAATTTTCGTTGTGGCCAGCCGCTGCAGAACAAGGTTCAGCTGAAG
GGCAGAGATCTGCTGACTCTGAAGAACTTCACTGGGGAAGAAATCAAGTATATGTT
ATGGCTGTCCGCGGATCTGAAATTTCGAATCAAGCAGAAGGGCGAATATCTTCCCC
TGCTGCAAGGGAAATCCTTGGGCATGATTTTTGAGAAGAGGAGCACTAGGACTAGA
TTGTCAACAGAAACAGGCTTTGCTTTGTTGGGCGGACATCCCTGCTTTCTGACGACA
CAGGATATCCACCTCGGCGTAAACGAGTCCCTCACCGACACTGCTAGGGTACTGAG
CAGCATGGCCGACGCTGTGCTAGCCCGGGTTTACAAGCAGTCAGACCTGGACACCC
TTGCCAAGGAAGCTTCTATTCCAATTATCAACGGCCTGAGTGACCTGTATCACCCTA
TTCAAATACTCGCCGACTATTTGACGCTTCAAGAACATTACAGCAGCCTCAAGGGCT
TAACCTTGAGTTGGATAGGCGACGGCAACAATATCCTGCATTCCATTATGATGTCTG
CCGCTAAGTTTGGCATGCATCTACAAGCCGCAACACCCAAGGGCTATGAACCCGAC
GCTAGCGTGACCAAGCTGGCCGAGCAGTATGCTAAGGAAAATGGCACAAAGCTCCT
TCTTACCAACGATCCCCTGGAGGCTGCTCACGGCGGCAACGTGCTGATTACCGATAC
ATGGATTAGCATGGGCCAGGAGGAGGAGAAAAAGAAGCGGCTCCAGGCTTTTCAA
GGCTATCAGGTCACCATGAAAACTGCAAAGGTCGCTGCCTCCGACTGGACTTTCCTG
CATTGTCTACCCCGCAAGCCTGAGGAAGTGGACGATGAGGTGTTCTACTCCCCACG
GAGTCTGGTGTTCCCGGAAGCAGAGAATCGGAAGTGGACCATCATGGCTGTCATGG
TGTCGCTCTTGACTGACTATTCTCCCCAACTGCAAAAACCCAAGTTTTAG
>pARM575 (SEQ ID NO: 140)
ATGCTTTTCAATCTGAGGATCCTGCTGAACAACGCTGCTTTTCGCAACGGTCATAAC
TTTATGGTTCGCAATTTTCGTTGTGGCCAGCCGCTGCAGAACAAGGTTCAGCTGAAG
GGCAGAGATCTGCTGACTCTGAAGAACTTCACTGGGGAAGAAATCAAGTATATGTT
ATGGCTGTCCGCGGATCTGAAATTTCGAATCAAGCAGAAGGGCGAATATCTTCCCC
TGCTGCAAGGGAAATCCTTGGGCATGATTTTTGAGAAGAGGAGCACTAGGACTAGA
TTGTCAACAGAAACAGGCTTTGCTTTGTTGGGCGGACATCCCTGCTTTCTGACGACA
CAGGATATCCACCTCGGCGTAAACGAGTCCCTCACCGACACTGCTAGGGTACTGAG
CAGCATGGCCGACGCTGTGCTAGCCCGGGTTTACAAGCAGTCAGACCTGGACACCC
TTGCCAAGGAAGCTTCTATTCCAATTATCAACGGCCTGAGTGACCTGTATCACCCTA
TTCAAATACTCGCCGACTATTTGACGCTTCAAGAACATTACAGCAGCCTCAAGGGCT
TAACCTTGAGTTGGATAGGCGACGGCAACAATATCCTGCATTCCATTATGATGTCTG
CCGCTAAGTTTGGCATGCATCTACAAGCCGCAACACCCAAGGGCTATGAACCCGAC
GCTAGCGTGACCAAGCTGGCCGAGCAGTATGCTAAGGAAAATGGCACAAAGCTCCT
TCTTACCAACGATCCCCTGGAGGCTGCTCACGGCGGCAACGTGCTGATTACCGATAC
ATGGATTAGCATGGGCCAGGAGGAGGAGAAAAAGAAGCGGCTCCAGGCTTTTCAA
GGCTATCAGGTCACCATGAAAACTGCAAAGGTCGCTGCCTCCGACTGGACTTTCCTG
CATTGTCTACCCCGCAAGCCTGAGGAAGTGGACGATGAGGTGTTCTACTCCCCACG
GAGTCTGGTGTTCCCGGAAGCAGAGAATCGGAAGTGGACCATCATGGCTGTCATGG
TGTCGCTCTTGACTGACTATTCTCCCCAACTGCAAAAACCCAAGTTTTAG
>pARM708 (SEQ ID NO: 141)
ATGCTTTTTAATCTCCGCATCCTCCTTAACAACGCCGCGTTTAGAAACGGCCACAAC
TTCATGGTCCGGAACTTCAGATGTGGCCAGCCGCTTCAAAACAAGGTCCAGCTGAA
GGGCCGGGATCTTCTGACCCTGAAGAACTTTACTGGCGAAGAGATCAAGTACATGC
TCTGGCTCTCCGCGGACTTGAAGTTCCGCATTAAGCAGAAGGGGGAATACCTTCCG
CTGCTTCAAGGAAAGAGCCTCGGCATGATCTTTGAGAAGCGCTCAACCAGGACCCG
CCTTTCTACTGAAACTGGGTTCGCGCTGCTCGGTGGCCACCCCTGCTTCCTGACGAC
CCAGGACATCCACCTCGGAGTGAACGAATCCCTCACCGATACCGCCCGGGTGTTAT
CGAGCATGGCAGATGCCGTGCTGGCCAGGGTGTACAAACAGTCCGATCTGGACACT
CTGGCCAAGGAGGCGTCAATTCCCATCATCAACGGCCTGAGCGACCTGTACCACCC
AATCCAAATCCTGGCTGACTACCTGACCCTGCAAGAGCACTACAGCAGCCTGAAGG
GTCTGACCCTGTCATGGATTGGCGATGGAAACAATATTCTGCACTCCATCATGATGT
CCGCCGCGAAGTTCGGAATGCATCTGCAAGCCGCCACGCCAAAAGGATACGAACCG
GATGCGTCCGTGACGAAGTTGGCGGAACAGTACGCGAAGGAGAACGGAACCAAGC
TTCTGCTGACTAACGACCCCCTCGAGGCTGCGCATGGGGGCAACGTGCTGATTACC
GACACCTGGATCTCCATGGGGCAGGAGGAAGAGAAGAAGAAGAGACTGCAGGCAT
TCCAGGGGTACCAGGTCACCATGAAAACCGCAAAAGTGGCAGCTTCGGACTGGACT
TTCCTGCATTGCCTGCCGAGGAAGCCGGAGGAAGTCGACGACGAAGTGTTCTACTC
GCCTCGGTCCCTGGTGTTCCCCGAGGCCGAAAACCGGAAGTGGACCATCATGGCCG
TGATGGTGTCCTTGCTGACTGACTATAGCCCGCAGCTGCAGAAGCCTAAGTTCTAG
>pARM709 (SEQ ID NO: 142)
ATGCTTTTCAACCTGAGAATCCTCTTGAACAATGCTGCTTTTCGGAATGGCCACAAC
TTTATGGTTCGGAACTTCCGTTGCGGCCAGCCTTTACAAAACAAGGTCCAGCTGAAG
GGCCGGGATTTGCTCACACTGAAGAACTTTACTGGAGAAGAGATCAAGTACATGCT
GTGGCTGTCGGCCGACCTGAAGTTCAGGATCAAGCAGAAGGGAGAATACCTTCCGC
TGCTTCAAGGAAAGAGCCTCGGCATGATCTTTGAGAAGCGCTCAACCAGGACCCGC
CTTTCTACTGAAACTGGGTTCGCGCTGCTCGGTGGCCACCCCTGCTTCCTGACGACC
CAGGACATCCACCTCGGAGTGAACGAATCCCTCACCGATACCGCCCGGGTGTTATC
GAGCATGGCAGATGCCGTGCTGGCCAGGGTGTACAAACAGTCCGATCTCGATACCT
TGGCAAAGGAGGCTTCCATTCCCATCATCAACGGCCTGAGCGACCTGTACCACCCA
ATCCAAATCCTGGCTGACTACCTGACCCTGCAAGAGCACTACAGCAGCCTGAAGGG
TCTGACCCTGTCATGGATTGGCGATGGAAACAATATTCTGCACTCCATCATGATGTC
CGCCGCGAAGTTCGGAATGCATCTGCAAGCCGCCACTCCAAAAGGATACGAACCGG
ATGCGTCCGTGACCAAGTTGGCGGAACAGTACGCGAAGGAGAACGGAACCAAGCT
TCTGCTGACTAACGACCCCCTCGAGGCTGCGCATGGGGGCAACGTGCTGATTACCG
ACACCTGGATCTCCATGGGGCAGGAGGAAGAGAAGAAGAAGAGACTGCAGGCATT
CCAGGGGTACCAGGTCACCATGAAAACCGCAAAAGTGGCAGCTTCGGACTGGACTT
TCCTGCATTGCCTGCCGAGGAAGCCGGAGGAAGTCGACGACGAAGTGTTCTACTCG
CCTCGGTCCCTGGTGTTCCCCGAGGCCGAAAACCGGAAGTGGACCATCATGGCCGT
GATGGTGTCCTTGCTGACTGACTATAGCCCGCAGCTGCAGAAGCCTAAGTTCTAG
>pARM710 (SEQ ID NO: 143)
ATGCTTTTCAACCTGAGAATCCTCTTGAACAATGCTGCTTTTCGGAATGGCCACAAC
TTTATGGTTCGGAACTTCCGTTGCGGCCAGCCTTTACAAAACAAGGTCCAGCTGAAG
GGCCGGGATTTGCTCACACTAAAGAACTTTACTGGAGAAGAGATCAAGTACATGCT
ATGGCTGTCGGCCGACCTGAAGTTCCGTATCAAGCAGAAGGGAGAATACCTTCCGC
TGCTTCAAGGAAAGAGCCTCGGCATGATCTTTGAGAAGCGCTCAACCAGGACCCGC
CTTTCTACTGAAACTGGGTTCGCGCTGCTCGGTGGCCACCCCTGCTTCCTGACGACC
CAGGACATCCACCTCGGAGTGAACGAATCCCTCACCGATACCGCCCGGGTGTTATC
GAGCATGGCAGATGCCGTGCTGGCCAGGGTGTACAAACAGTCCGATCTCGATACCT
TGGCAAAGGAGGCTTCCATTCCCATCATCAACGGCCTGAGCGACCTGTACCACCCA
ATCCAAATCCTGGCTGACTACCTGACCCTGCAAGAGCACTACAGCAGCCTGAAGGG
TCTGACCCTGTCATGGATTGGCGATGGAAACAATATTCTGCACTCCATCATGATGTC
CGCCGCGAAGTTCGGAATGCATCTGCAAGCCGCCACTCCAAAAGGATACGAACCGG
ATGCATCCGTGACCAAGTTGGCGGAACAGTACGCGAAGGAGAACGGAACCAAGCT
CCTGCTGACTAACGACCCGCTCGAGGCTGCGCATGGGGGTAACGTGCTGATTACGG
ACACCTGGATCTCCATGGGGCAGGAGGAAGAGAAGAAGAAGAGACTGCAGGCATT
CCAGGGGTACCAGGTCACCATGAAAACCGCAAAAGTGGCAGCTTCGGACTGGACTT
TCCTGCATTGCCTGCCGAGGAAGCCGGAGGAAGTCGACGACGAAGTGTTCTACTCG
CCTCGGTCCCTGGTGTTCCCCGAGGCCGAAAACCGGAAGTGGACCATCATGGCCGT
GATGGTGTCCTTGCTGACTGACTATAGCCCGCAGCTGCAGAAGCCTAAGTTCTAG
>pARM711 (SEQ ID NO: 144)
ATGCTTTTCAACCTGAGAATCCTCTTGAACAATGCTGCTTTTCGGAATGGCCACAAC
TTTATGGTTCGGAACTTCCGTTGCGGCCAGCCTTTACAAAACAAGGTCCAGCTGAAG
GGCCGGGATTTGCTCACACTAAAGAACTTTACTGGAGAAGAGATCAAGTACATGCT
ATGGCTGTCGGCCGACCTGAAGTTCCGTATCAAGCAGAAGGGAGAATACCTTCCGC
TGCTTCAAGGAAAGAGCCTCGGCATGATCTTTGAGAAGCGCTCAACCAGGACCCGC
CTTTCTACTGAAACTGGGTTCGCGCTGCTCGGTGGCCACCCCTGCTTCCTGACGACC
CAGGACATCCACCTCGGAGTGAACGAATCCCTCACCGATACCGCCCGGGTGTTATC
GAGCATGGCAGATGCCGTGCTGGCCAGGGTGTACAAACAGTCCGATCTCGATACCT
TGGCAAAGGAGGCTTCCATTCCCATCATCAACGGCCTGAGCGACCTGTACCACCCA
ATCCAAATCCTGGCTGACTACCTGACCCTGCAAGAGCACTACAGCAGCCTGAAGGG
TCTGACCCTGTCATGGATTGGCGATGGAAACAATATTCTGCACTCCATCATGATGTC
CGCCGCGAAGTTCGGAATGCATCTGCAAGCCGCCACTCCAAAAGGATACGAACCGG
ATGCGTCCGTGACCAAGTTGGCGGAACAGTACGCGAAGGAGAACGGAACCAAGCT
TCTGCTGACTAACGACCCCCTCGAGGCTGCGCATGGGGGCAACGTGCTGATTACCG
ACACCTGGATCTCCATGGGGCAGGAGGAAGAGAAGAAGAAGAGACTGCAGGCATT
CCAGGGGTACCAGGTCACCATGAAAACCGCAAAAGTGGCAGCTTCGGACTGGACTT
TCCTGCATTGCCTGCCGAGGAAGCCGGAGGAAGTCGACGACGAAGTGTTCTACTCG
CCTCGGTCCCTGGTGTTCCCCGAGGCCGAAAACCGGAAGTGGACCATCATGGCCGT
GATGGTGTCCTTGCTGACTGACTATAGCCCGCAGCTGCAGAAGCCTAAGTTCTAG
>pARM712 (SEQ ID NO: 145)
ATGCTGTTCAACCTGCGAATCCTGCTGAACAACGCCGCTTTTCGGAACGGGCACAA
CTTTATGGTGAGGAACTTTCGCTGCGGACAGCCCCTCCAGAATAAGGTCCAGCTGA
AGGGCAGGGACCTGCTGACCCTGAAAAATTTCACAGGGGAGGAAATCAAGTATATG
CTGTGGCTGTCAGCTGATCTGAAGTTCCGGATCAAGCAGAAGGGCGAATATCTGCC
TCTGCTCCAGGGCAAAAGCCTGGGGATGATCTTCGAAAAGCGCAGTACTCGGACCA
GACTGTCAACCGAGACTGGATTCGCTCTGCTGGGAGGACACCCTTGTTTTCTGACCA
CTCAGGACATTCACCTGGGAGTGAACGAGTCCCTGACCGACACTGCTCGCGTCCTG
AGCTCTATGGCCGACGCTGTGCTGGCTCGAGTCTACAAACAGTCCGACCTGGATAC
CCTGGCCAAGGAAGCTTCTATCCCAATTATTAACGGCCTGTCAGACCTGTATCACCC
CATCCAGATTCTGGCCGATTACCTGACCCTCCAGGAGCACTATTCTAGTCTGAAAGG
GCTGACACTGAGTTGGATTGGGGACGGAAACAATATCCTGCACTCTATTATGATGTC
AGCCGCCAAGTTTGGAATGCACCTCCAGGCTGCAACCCCAAAAGGCTACGAACCCG
ATGCCTCAGTGACAAAGCTGGCTGAACAGTACGCCAAAGAGAACGGCACTAAGCTG
CTGCTGACCAACGACCCTCTGGAGGCCGCTCACGGAGGCAACGTGCTGATCACCGA
TACCTGGATTAGTATGGGACAGGAGGAAGAGAAGAAGAAGCGGCTCCAGGCCTTC
CAGGGCTACCAGGTGACAATGAAAACCGCTAAGGTCGCAGCCAGCGATTGGACCTT
TCTGCACTGCCTGCCCAGAAAGCCCGAAGAGGTGGACGACGAGGTCTTCTACTCTC
CCAGAAGCCTGGTGTTTCCCGAAGCTGAGAATAGGAAGTGGACAATTATGGCAGTG
ATGGTCAGCCTGCTGACTGATTATTCACCTCAGCTCCAGAAACCAAAGTTCTGA
>pARM713 (SEQ ID NO: 146)
ATGCTGTTCAACCTGCGCATCCTGCTGAACAACGCCGCCTTCCGCAACGGCCACAAC
TTCATGGTGCGCAACTTCCGCTGCGGCCAGCCCCTGCAGAACAAGGTGCAGCTGAA
GGGCCGCGACCTGCTGACCCTGAAGAACTTCACCGGCGAGGAGATCAAGTACATGC
TGTGGCTGAGCGCCGACCTGAAGTTCCGCATCAAGCAGAAGGGCGAGTACCTGCCC
CTGCTGCAGGGCAAGAGCCTGGGCATGATCTTCGAGAAGCGCAGCACCCGCACCCG
CCTGAGCACCGAGACAGGCCTGGCCCTGCTGGGCGGCCACCCCTGCTTCCTGACCA
CCCAGGACATCCACCTGGGCGTGAACGAGAGCCTGACCGACACCGCCCGCGTGCTG
AGCAGCATGGCCGACGCCGTGCTGGCCCGCGTGTACAAGCAGAGCGACCTGGACAC
CCTGGCCAAGGAGGCCAGCATCCCCATCATCAACGGCCTGAGCGACCTGTACCACC
CCATCCAGATCCTGGCCGACTACCTGACCCTGCAGGAGCACTACAGCAGCCTGAAG
GGCCTGACCCTGAGCTGGATCGGCGACGGCAACAACATCCTGCACAGCATCATGAT
GAGCGCCGCCAAGTTCGGCATGCACCTGCAGGCCGCCACCCCCAAGGGCTACGAGC
CCGACGCCAGCGTGACCAAGCTGGCCGAGCAGTACGCCAAGGAGAACGGCACCAA
GCTGCTGCTGACCAACGACCCCCTGGAGGCCGCCCACGGCGGCAACGTGCTGATCA
CCGACACCTGGATCAGCATGGGCCAGGAGGAGGAGAAGAAGAAGCGCCTGCAGGC
CTTCCAGGGCTACCAGGTGACCATGAAGACCGCCAAGGTGGCCGCCAGCGACTGGA
CCTTCCTGCACTGCCTGCCCCGCAAGCCCGAGGAGGTGGACGACGAGGTGTTCTAC
AGCCCCCGCAGCCTGGTGTTCCCCGAGGCCGAGAACCGCAAGTGGACCATCATGGC
CGTGATGGTGAGCCTGCTGACCGACTACAGCCCCCAGCTGCAGAAGCCCAAGTTCT
GA
>pARM714 (SEQ ID NO: 147)
ATGCTGTTCAACCTGCGAATCCTGCTGAACAACGCCGCTTTTCGGAACGGGCACAA
CTTTATGGTGAGGAACTTTCGCTGCGGACAGCCCCTCCAGAATAAGGTCCAGCTGA
AGGGCAGGGACCTGCTGACCCTGAAAAATTTCACAGGGGAGGAAATCAAGTATATG
CTGTGGCTGTCAGCTGATCTGAAGTTCCGGATCAAGCAGAAGGGCGAATATCTGCC
TCTGCTCCAGGGCAAAAGCCTGGGGATGATCTTCGAAAAGCGCAGTACTCGGACCA
GACTGTCAACCGAGACTGGATTCGCTCTGCTGGGAGGACACCCTTGTTTTCTGACCA
CTCAGGACATTCACCTGGGAGTGAACGAGTCCCTGACCGACACTGCTCGCGTCCTG
AGCTCTATGGCCGACGCTGTGCTAGCTCGAGTCTACAAACAGTCCGACCTGGATAC
CCTGGCCAAGGAAGCTTCTATCCCAATTATTAACGGCCTGTCAGACCTGTATCACCC
CATCCAGATTCTGGCCGATTACCTGACCCTCCAGGAGCACTATTCTAGTCTGAAAGG
GCTGACACTGAGTTGGATTGGGGACGGAAACAATATCCTGCACTCTATTATGATGTC
AGCCGCCAAGTTTGGAATGCACCTCCAGGCTGCAACCCCAAAAGGCTACGAACCCG
ATGCCTCAGTGACAAAGCTGGCTGAACAGTACGCCAAAGAGAACGGCACTAAGCTG
CTGCTGACCAACGACCCTCTGGAGGCCGCTCACGGAGGCAACGTGCTGATCACCGA
TACCTGGATTAGTATGGGACAGGAGGAAGAGAAGAAGAAGCGGCTCCAGGCCTTC
CAGGGCTACCAGGTGACAATGAAAACCGCTAAGGTCGCAGCCAGCGATTGGACCTT
TCTGCACTGCCTGCCCAGAAAGCCCGAAGAGGTGGACGACGAGGTCTTCTACTCTC
CCAGAAGCCTGGTGTTTCCCGAAGCTGAGAATAGGAAGTGGACAATTATGGCAGTG
ATGGTCAGCCTGCTGACTGATTATTCACCTCAGCTCCAGAAACCAAAGTTCTGA
>pARM715 (SEQ ID NO: 148)
ATGCTGTTCAACCTGCGAATCCTGCTGAACAATGCCGCTTTTCGGAACGGGCACAAT
TTCATGGTGAGGAACTTTCGCTGCGGACAGCCCCTCCAGAACAAGGTCCAGCTGAA
GGGCAGGGACCTGCTGACCCTGAAAAATTTCACAGGGGAGGAAATCAAGTACATGC
TGTGGCTGTCAGCCGATCTGAAGTTCCGGATCAAGCAGAAGGGCGAATATCTGCCT
CTGCTCCAGGGCAAAAGCCTGGGGATGATCTTCGAAAAGCGCAGTACTCGGACCAG
ACTGTCAACAGAGACTGGATTCGCACTGCTGGGAGGACACCCATGTTTTCTGACCA
CACAGGACATTCATCTGGGAGTGAACGAGTCCCTGACCGACACAGCACGCGTCCTG
AGCTCCATGGCTGATGCAGTGCTGGCTCGAGTCTACAAACAGTCTGACCTGGATAC
CCTGGCCAAGGAAGCTTCTATCCCAATCATTAATGGCCTGAGTGACCTGTATCACCC
CATCCAGATTCTGGCCGATTACCTGACCCTCCAGGAGCATTATTCTAGTCTGAAAGG
GCTGACACTGAGCTGGATTGGGGACGGAAACAATATCCTGCACTCCATTATGATGA
GCGCCGCCAAGTTTGGAATGCACCTCCAGGCTGCAACCCCAAAAGGCTACGAACCC
GATGCCTCCGTGACAAAGCTGGCAGAACAGTATGCCAAAGAGAACGGCACTAAGCT
GCTGCTGACCAATGACCCTCTGGAGGCCGCTCACGGAGGCAACGTGCTGATCACTG
ATACCTGGATTAGTATGGGACAGGAGGAAGAGAAGAAGAAGCGGCTCCAGGCCTT
CCAGGGCTACCAGGTGACAATGAAAACTGCTAAGGTCGCAGCCAGCGACTGGACCT
TTCTGCATTGCCTGCCCAGAAAGCCTGAAGAGGTGGACGATGAGGTCTTCTACTCAC
CCAGAAGCCTGGTGTTTCCTGAAGCTGAGAATAGGAAGTGGACAATCATGGCAGTG
ATGGTCAGCCTGCTGACTGATTATTCCCCTCAGCTCCAGAAACCAAAGTTCTGA
>pARM716 (SEQ ID NO: 149)
ATGCTTTTCAACCTTCGCATTCTCCTCAACAACGCCGCGTTTAGAAACGGACACAAC
TTCATGGTCCGCAACTTCCGCTGCGGACAGCCGCTGCAGAACAAGGTCCAGCTCAA
GGGTCGGGATCTCCTGACGCTGAAGAACTTTACCGGCGAAGAGATTAAGTACATGC
TGTGGCTGTCCGCCGACCTTAAGTTCCGGATCAAGCAGAAGGGCGAATACCTTCCC
CTGCTGCAAGGAAAGTCCCTGGGCATGATCTTCGAGAAGCGCAGTACCAGAACCAG
ACTCTCCACTGAAACCGGGTTCGCGCTGCTTGGCGGCCACCCGTGTTTCCTCACTAC
GCAAGACATCCATCTTGGCGTGAACGAGTCCCTTACCGACACCGCCAGGGTGCTGT
CAAGCATGGCCGACGCCGTCCTTGCGCGCGTGTACAAGCAGTCAGACCTTGATACT
CTGGCCAAGGAAGCCTCCATCCCTATTATCAACGGCCTATCCGACCTTTACCACCCG
ATCCAGATCCTCGCTGACTACCTGACCCTGCAAGAACACTACAGCAGCCTCAAGGG
ACTGACTCTGTCCTGGATCGGCGACGGGAACAACATCCTGCACTCAATCATGATGA
GCGCAGCCAAGTTCGGCATGCATCTCCAAGCCGCTACACCCAAGGGTTATGAACCG
GACGCCTCTGTGACCAAGTTGGCAGAACAGTACGCCAAGGAGAACGGTACTAAGCT
CCTTTTAACCAACGACCCCCTCGAAGCAGCCCATGGCGGGAATGTGCTCATTACCG
ATACCTGGATTTCGATGGGCCAGGAGGAGGAGAAGAAGAAGCGGCTGCAGGCGTT
CCAGGGCTACCAGGTCACCATGAAAACTGCCAAAGTGGCCGCCTCGGATTGGACCT
TTCTCCACTGCCTGCCTCGGAAGCCTGAGGAGGTGGACGACGAAGTGTTCTACTCCC
CACGGTCCCTCGTGTTCCCCGAGGCCGAAAATAGGAAGTGGACCATCATGGCCGTG
ATGGTGTCCCTCTTGACCGATTACAGCCCGCAGCTTCAGAAGCCTAAATTCTAG
>pARM717 (SEQ ID NO: 150)
ATGCTTTTCAATCTTCGCATCCTGTTGAACAACGCCGCCTTCCGCAATGGTCACAAC
TTCATGGTCCGGAACTTCAGATGTGGACAGCCTCTCCAAAACAAGGTCCAGCTGAA
GGGAAGGGACCTCTTAACCCTCAAAAACTTTACTGGAGAGGAGATCAAGTACATGC
TGTGGCTTAGCGCCGACCTTAAGTTCCGGATCAAGCAGAAGGGAGAGTACCTCCCG
CTGCTGCAAGGAAAGAGTCTTGGAATGATCTTCGAGAAGCGGTCCACCAGAACTCG
CCTCTCCACTGAAACCGGATTCGCACTCCTGGGTGGACACCCGTGCTTTCTGACCAC
CCAAGACATCCACCTCGGAGTGAACGAGAGCCTCACGGACACCGCGAGAGTGCTGT
CATCCATGGCCGACGCCGTGCTTGCACGGGTCTACAAGCAGTCCGATCTGGACACT
CTTGCCAAGGAAGCCTCCATTCCTATCATTAACGGTCTGTCGGATCTGTACCACCCG
ATTCAGATCCTTGCGGACTACCTCACACTTCAAGAACACTATTCAAGCCTAAAGGGT
CTGACCCTGTCCTGGATCGGAGATGGAAACAACATTCTCCATTCCATCATGATGAGC
GCTGCCAAGTTCGGAATGCATCTCCAAGCAGCGACTCCTAAGGGTTACGAGCCGGA
CGCCTCAGTGACTAAGCTGGCCGAGCAGTACGCCAAGGAGAACGGTACCAAACTGT
TGCTTACTAACGACCCGCTTGAAGCGGCCCATGGAGGAAACGTGCTGATTACCGAC
ACCTGGATTTCGATGGGACAGGAAGAGGAGAAGAAGAAGCGGCTCCAGGCGTTCC
AGGGATACCAGGTCACCATGAAAACGGCCAAAGTGGCCGCTAGCGATTGGACCTTT
CTGCACTGCCTCCCGCGCAAGCCTGAAGAAGTGGACGACGAAGTGTTCTACTCCCC
TCGCTCTCTTGTGTTCCCGGAAGCCGAAAACAGGAAGTGGACCATCATGGCCGTGA
TGGTGTCCCTCCTGACCGATTACAGCCCGCAGCTGCAGAAGCCTAAGTTCTAG
>pARM718 (SEQ ID NO: 151)
ATGCTTTTCAATCTCCGCATCCTCCTCAACAACGCCGCGTTTAGAAACGGCCACAAC
TTCATGGTCCGGAACTTCAGATGTGGCCAGCCGCTTCAGAACAAGGTCCAGCTCAA
GGGCCGGGATCTTCTGACCCTGAAGAACTTTACTGGCGAAGAAATCAAGTACATGC
TCTGGCTCTCCGCCGACTTGAAGTTCCGCATTAAGCAGAAGGGGGAATACCTTCCGC
TGCTGCAAGGAAAGTCGCTCGGCATGATCTTTGAGAAGCGCTCAACCCGCACCAGG
CTGTCCACTGAAACCGGGTTCGCGCTGCTTGGTGGCCACCCCTGCTTCCTGACCACC
CAAGACATTCACCTCGGAGTGAACGAATCGCTCACTGATACTGCCCGGGTGCTGTC
GTCGATGGCCGATGCAGTGCTGGCCAGGGTGTACAAACAGTCCGATCTGGACACTC
TGGCCAAGGAGGCGTCCATCCCTATTATCAACGGCCTTTCCGACCTCTACCACCCGA
TTCAGATCCTTGCCGATTACCTCACCCTGCAAGAACACTACTCGTCACTGAAGGGTC
TGACCTTGTCCTGGATCGGCGACGGCAACAACATCCTCCATTCCATTATGATGTCCG
CCGCCAAATTCGGCATGCATCTTCAAGCCGCAACCCCTAAGGGTTACGAGCCGGAC
GCTTCCGTGACCAAGCTCGCCGAGCAGTACGCTAAGGAGAACGGAACCAAGCTTCT
GCTGACTAACGACCCCCTAGAGGCAGCCCACGGGGGCAACGTGCTTATTACTGACA
CCTGGATCTCCATGGGACAGGAAGAAGAGAAGAAGAAGCGGTTACAGGCGTTCCA
GGGCTATCAGGTCACCATGAAAACCGCCAAGGTCGCTGCCTCGGACTGGACCTTCC
TGCATTGCCTGCCTCGCAAGCCCGAAGAAGTGGACGACGAGGTGTTCTACTCGCCA
CGGTCCCTTGTGTTCCCTGAGGCCGAGAATAGAAAGTGGACCATTATGGCCGTGAT
GGTGTCCCTTCTCACCGACTACTCGCCGCAACTGCAGAAACCCAAGTTCTAG
>pARM719 (SEQ ID NO: 152)
ATGCTTTTCAATCTTCGCATCCTCCTCAACAACGCCGCCTTCCGGAACGGTCACAAC
TTCATGGTCCGGAACTTCCGCTGCGGCCAGCCGCTCCAAAACAAAGTGCAGCTTAA
GGGCCGCGATCTCCTGACCCTGAAGAACTTCACCGGAGAGGAAATCAAGTACATGC
TGTGGCTCTCGGCGGACCTGAAGTTTAGGATTAAGCAGAAGGGGGAGTATCTGCCG
CTGCTCCAAGGGAAGTCCCTTGGCATGATCTTCGAAAAGAGGTCCACCCGGACTCG
GCTCAGCACCGAAACAGGTTTTGCACTTCTGGGGGGCCACCCGTGCTTCCTGACGAC
CCAGGACATCCATCTGGGTGTCAACGAGAGTTTGACCGACACTGCCAGAGTGCTGT
CATCCATGGCGGACGCGGTGCTCGCGAGAGTGTACAAGCAGTCCGATCTTGACACC
CTGGCAAAAGAGGCTTCAATCCCGATCATTAACGGACTCTCGGATCTGTACCACCCT
ATCCAAATCTTGGCCGACTACCTGACCCTGCAAGAACACTACAGCTCCCTGAAGGG
CCTGACTCTTTCCTGGATTGGCGATGGAAACAACATTCTCCATTCTATTATGATGTC
CGCCGCCAAGTTCGGCATGCACCTTCAAGCCGCCACCCCGAAGGGCTACGAACCTG
ACGCCTCCGTGACTAAGCTAGCCGAACAGTACGCTAAGGAGAACGGCACTAAGCTT
CTCCTTACCAACGATCCGCTGGAGGCGGCCCATGGCGGAAATGTGCTTATCACCGA
CACCTGGATTAGCATGGGGCAGGAAGAAGAGAAGAAGAAACGGCTCCAGGCATTC
CAGGGCTACCAGGTCACCATGAAAACTGCCAAGGTCGCCGCTAGCGACTGGACCTT
CCTCCACTGTCTGCCTCGCAAGCCTGAAGAAGTGGACGACGAGGTGTTCTACTCCCC
GCGCTCCCTCGTGTTTCCTGAGGCCGAGAACAGAAAGTGGACCATCATGGCCGTGA
TGGTGTCATTACTTACGGACTACAGCCCGCAGCTGCAGAAGCCGAAGTTCTAG
>pARM720 (SEQ ID NO: 153)
ATGCTTTTTAACTTGAGAATCCTTCTGAACAACGCCGCTTTCCGCAACGGTCATAAC
TTCATGGTCCGGAACTTCAGATGTGGCCAGCCCCTCCAAAACAAAGTGCAGCTGAA
GGGCCGGGACCTTCTTACGCTGAAGAATTTCACCGGCGAAGAAATCAAGTACATGC
TCTGGCTGTCCGCCGATCTTAAGTTCCGCATTAAGCAGAAGGGGGAATACCTCCCGC
TGCTGCAAGGGAAGTCGCTGGGCATGATTTTTGAGAAGCGGTCAACTCGCACCCGC
CTGTCCACTGAAACTGGATTCGCACTGCTCGGTGGCCATCCCTGCTTCCTGACCACC
CAAGACATCCACCTCGGCGTGAACGAGTCCCTGACTGACACCGCCCGGGTCTTATC
CTCGATGGCCGATGCTGTGCTTGCGAGGGTGTACAAGCAGTCCGACCTCGACACAC
TCGCGAAGGAGGCCTCCATCCCCATCATCAACGGCCTGTCCGACCTTTACCACCCAA
TTCAGATCCTCGCCGATTACCTGACCCTGCAAGAGCACTACTCGTCGCTCAAGGGGC
TTACCCTCTCGTGGATTGGCGACGGCAACAACATCCTTCACTCCATCATGATGTCGG
CAGCGAAGTTCGGCATGCATCTGCAAGCCGCCACGCCTAAGGGTTATGAACCGGAT
GCCTCAGTGACCAAGCTCGCCGAACAGTACGCGAAAGAGAATGGAACCAAGCTACT
TCTGACCAACGACCCCCTGGAGGCCGCTCACGGCGGCAACGTCCTCATTACCGATA
CTTGGATTTCGATGGGACAGGAAGAGGAAAAGAAGAAGAGACTGCAGGCGTTCCA
GGGATACCAGGTCACCATGAAAACTGCCAAAGTGGCAGCCTCCGACTGGACCTTCC
TTCACTGCCTGCCGAGGAAGCCTGAAGAGGTGGACGACGAGGTGTTCTACTCCCCG
CGCTCCTTGGTGTTTCCTGAGGCCGAAAACCGGAAGTGGACTATCATGGCCGTGAT
GGTGTCCCTCCTCACCGACTACTCGCCGCAACTGCAGAAGCCTAAGTTCTAG
>pARM721 (SEQ ID NO: 154)
ATGTTATTCAACCTTAGAATTCTCCTTAACAACGCCGCCTTCCGGAATGGGCATAAC
TTTATGGTCCGCAATTTCCGCTGTGGACAGCCTCTGCAAAACAAGGTCCAGCTCAAG
GGCCGGGATCTGCTGACTCTCAAGAACTTCACTGGGGAAGAAATCAAGTACATGCT
CTGGCTGAGCGCCGACCTCAAGTTCCGCATCAAGCAGAAGGGAGAGTACCTCCCGC
TGCTCCAAGGGAAGTCCCTGGGCATGATCTTCGAGAAGAGATCCACCCGCACCAGA
CTTTCCACTGAGACTGGCTTCGCCTTGCTGGGAGGCCACCCATGCTTCCTGACGACC
CAGGACATTCACCTTGGCGTGAACGAGTCCCTGACTGACACCGCAAGGGTGTTGTC
CTCGATGGCCGACGCCGTGCTTGCCCGGGTGTACAAGCAGAGCGATCTTGACACCC
TGGCTAAGGAAGCTTCCATTCCCATCATCAACGGTCTGAGCGACCTGTACCACCCGA
TTCAGATCCTGGCGGACTACCTAACCCTGCAAGAGCACTATAGCTCCCTGAAGGGC
CTCACACTTTCATGGATCGGCGACGGCAACAACATCCTGCACTCTATTATGATGAGC
GCTGCCAAATTCGGCATGCACCTCCAAGCCGCCACGCCTAAAGGCTACGAGCCCGA
CGCCTCGGTGACCAAGCTTGCGGAGCAGTACGCGAAGGAAAACGGCACCAAGCTG
CTTCTCACCAACGATCCTCTGGAAGCGGCCCATGGTGGCAACGTGCTCATTACCGAC
ACTTGGATCTCCATGGGACAGGAGGAGGAAAAGAAGAAGCGGCTCCAGGCGTTTC
AGGGTTACCAGGTCACCATGAAAACCGCCAAGGTCGCAGCCTCCGACTGGACCTTC
CTTCATTGCCTTCCGCGCAAGCCCGAAGAAGTGGACGATGAAGTGTTTTACTCACCT
CGGTCACTCGTGTTCCCGGAAGCAGAGAACAGGAAATGGACCATTATGGCCGTGAT
GGTGTCCCTGCTCACCGATTACAGTCCGCAACTGCAGAAGCCCAAGTTCTAG
>pARM722 (SEQ ID NO: 155)
ATGCTTTTCAATCTCCGCATCCTCCTTAACAACGCCGCGTTTAGAAACGGCCACAAC
TTCATGGTCCGGAACTTCAGATGTGGCCAGCCGCTTCAAAACAAGGTCCAGCTGAA
GGGCCGGGATCTTCTGACCCTGAAGAACTTTACTGGCGAAGAGATCAAGTACATGC
TCTGGCTCTCCGCGGACTTGAAGTTCCGCATTAAGCAGAAGGGGGAATACCTTCCG
CTGCTTCAAGGAAAGAGCCTCGGCATGATCTTTGAGAAGCGCTCAACCAGGACCCG
CCTTTCTACTGAAACTGGGTTCGCGCTGCTCGGTGGCCACCCCTGCTTCCTGACGAC
CCAGGACATCCACCTCGGAGTGAACGAATCCCTCACCGATACCGCCCGGGTGTTAT
CGAGCATGGCAGATGCCGTGCTGGCCAGGGTGTACAAACAGTCCGATCTGGACACT
CTGGCCAAGGAGGCGTCAATTCCTATTATCAACGGCCTTAGTGACCTCTACCATCCG
ATTCAAATCCTGGCCGATTACCTCACCCTGCAAGAACACTACAGCTCCCTGAAGGGT
CTGACATTGTCCTGGATCGGCGACGGCAACAACATTCTCCATTCCATCATGATGTCC
GCCGCAAAATTCGGCATGCATCTTCAAGCCGCCACGCCTAAGGGTTACGAACCCGA
CGCTTCCGTGACTAAGCTCGCCGAGCAGTACGCTAAGGAGAACGGAACCAAGCTGC
TGCTGACTAACGACCCGCTAGAAGCAGCCCACGGGGGCAACGTGCTTATTACTGAC
ACCTGGATCTCCATGGGCCAGGAGGAAGAGAAAAAGAAGCGGCTGCAGGCGTTCC
AGGGATATCAGGTCACCATGAAAACCGCCAAGGTCGCTGCCTCCGACTGGACCTTC
CTGCACTGCCTGCCTCGCAAGCCTGAAGAAGTGGACGACGAGGTGTTCTACTCGCC
ACGGAGCCTCGTGTTCCCCGAGGCCGAGAATAGAAAGTGGACCATCATGGCCGTGA
TGGTGTCACTTCTCACCGACTACAGCCCGCAGCTTCAGAAGCCCAAGTTCTAG
>pARM723 (SEQ ID NO: 156)
ATGCTTTTCAATCTCCGCATCCTCCTTAACAACGCCGCGTTTAGAAACGGCCACAAC
TTCATGGTCCGGAACTTCAGATGTGGCCAGCCGCTTCAAAACAAGGTCCAGCTTAA
GGGCCGGGATCTCCTCACCCTTAAAAACTTCACCGGCGAAGAGATCAAGTACATGC
TCTGGCTCTCCGCGGACCTTAAGTTCCGCATTAAGCAGAAGGGGGAATACCTTCCGC
TGCTTCAAGGAAAGAGCCTCGGCATGATCTTTGAGAAGCGCTCAACCAGGACCAGG
CTTTCTACTGAAACTGGGTTCGCGCTTCTCGGCGGTCATCCCTGCTTCCTCACGACCC
AAGACATCCACCTCGGAGTGAACGAATCCCTCACGGATACTGCCCGCGTGCTTTCG
AGCATGGCAGACGCCGTGCTCGCCCGGGTGTACAAACAGTCCGATCTCGACACTCT
CGCCAAGGAGGCGTCAATTCCTATTATCAACGGTCTTAGTGACCTTTACCACCCGAT
CCAGATCCTCGCCGATTACCTCACACTCCAAGAACACTACAGCTCCCTTAAGGGTCT
TACCCTCTCCTGGATCGGCGACGGCAACAACATTCTCCACTCCATCATGATGTCCGC
CGCAAAGTTCGGCATGCATCTTCAAGCCGCCACCCCGAAGGGCTACGAGCCTGATG
CTTCCGTGACTAAGCTCGCCGAGCAGTACGCTAAGGAGAACGGAACCAAGCTTCTT
CTCACTAACGACCCACTCGAAGCAGCCCATGGGGGCAACGTGCTTATCACTGACAC
CTGGATCTCCATGGGCCAGGAAGAAGAGAAGAAGAAGCGGCTCCAGGCGTTCCAG
GGATATCAGGTCACCATGAAAACCGCCAAGGTCGCTGCCTCCGACTGGACCTTTCTC
CACTGCCTCCCTCGCAAACCTGAAGAAGTGGACGACGAGGTGTTCTACTCGCCCCG
GAGCCTCGTGTTCCCCGAGGCCGAGAATAGAAAGTGGACCATTATGGCCGTGATGG
TGTCACTCCTCACCGACTACAGCCCGCAGCTTCAGAAGCCCAAGTTCTAG
>pARM724 (SEQ ID NO: 157)
ATGCTTTTCAATCTCCGCATCCTCCTTAACAACGCCGCGTTTAGAAACGGACATAAC
TTCATGGTCCGGAACTTCAGATGTGGACAGCCGCTTCAAAACAAGGTCCAGCTGAA
GGGTCGGGATCTTCTGACCCTGAAGAACTTTACCGGAGAAGAGATCAAGTACATGC
TCTGGCTCTCCGCGGACTTGAAGTTCCGCATTAAGCAGAAGGGAGAATACCTCCCG
CTGCTTCAAGGAAAGAGCCTCGGAATGATTTTTGAGAAGCGCTCAACCAGGACCCG
CCTTTCTACTGAAACTGGATTCGCGCTGCTGGGTGGACACCCCTGCTTCCTGACGAC
CCAGGACATCCACCTCGGAGTGAACGAATCCCTCACTGATACCGCCCGGGTGTTAT
CGAGCATGGCAGATGCCGTGCTGGCCAGGGTGTACAAACAGTCCGATCTGGACACT
CTGGCCAAGGAGGCGTCAATTCCTATCATCAACGGACTTAGTGACCTCTACCATCCG
ATTCAAATCCTGGCCGACTACCTCACCCTGCAAGAACACTACAGCTCCCTGAAGGG
TCTGACATTGTCCTGGATCGGAGATGGAAACAACATTCTCCACTCCATCATGATGTC
CGCCGCAAAATTCGGAATGCATCTTCAAGCCGCCACGCCTAAGGGTTACGAACCCG
ACGCTTCCGTGACTAAGCTCGCCGAGCAGTACGCTAAGGAGAACGGTACCAAGCTT
CTCCTGACCAACGACCCACTAGAAGCAGCCCACGGTGGAAACGTGCTTATTACTGA
CACTTGGATCTCCATGGGACAGGAGGAAGAGAAAAAGAAGCGGCTGCAGGCGTTC
CAGGGATATCAGGTCACCATGAAAACCGCCAAGGTCGCTGCCTCCGACTGGACCTT
CCTGCACTGCCTGCCTCGCAAGCCTGAAGAAGTGGACGACGAGGTGTTCTACTCGC
CGCGGAGCCTCGTGTTCCCCGAGGCCGAGAATAGAAAGTGGACCATCATGGCCGTG
ATGGTGTCACTGCTCACCGACTACAGCCCGCAGCTTCAGAAGCCCAAGTTCTAG
>pARM725 (SEQ ID NO: 158)
ATGCTTTTCAACCTCCGCATTCTCCTCAACAACGCTGCCTTCCGGAATGGACATAAC
TTCATGGTCCGGAACTTCAGATGCGGACAGCCGCTTCAGAACAAGGTCCAGCTTAA
GGGGAGAGATCTCCTTACCCTCAAAAACTTCACTGGCGAAGAAATCAAGTACATGC
TCTGGCTTAGTGCGGATCTCAAGTTCCGCATCAAGCAGAAGGGAGAATACCTCCCG
CTCCTTCAAGGAAAGAGCCTCGGCATGATTTTTGAGAAGAGGTCCACCAGAACTCG
CCTTTCAACCGAGACTGGGTTCGCCCTGCTTGGCGGTCACCCCTGCTTCCTCACTAC
CCAAGACATCCACCTCGGCGTGAACGAGAGCCTTACCGACACCGCCCGCGTGCTCT
CCTCAATGGCCGACGCTGTGCTCGCCCGGGTGTACAAGCAGTCCGACCTTGATACTC
TCGCCAAGGAGGCCTCCATCCCAATTATCAACGGGCTCTCTGATCTCTACCACCCTA
TCCAAATCCTCGCGGACTACCTCACCCTCCAAGAGCACTATAGCTCGCTCAAGGGCC
TCACCCTTTCCTGGATTGGCGACGGCAACAACATTCTTCACTCGATCATGATGTCCG
CCGCCAAGTTCGGCATGCATCTCCAAGCCGCGACCCCCAAGGGCTACGAGCCTGAC
GCATCCGTGACCAAGCTCGCCGAGCAGTACGCGAAGGAAAATGGCACCAAGCTTCT
TCTCACCAACGACCCCCTTGAGGCCGCTCATGGCGGCAACGTGCTCATCACTGACAC
TTGGATCAGCATGGGCCAGGAGGAGGAAAAGAAGAAGCGCCTTCAGGCATTCCAG
GGTTACCAGGTCACCATGAAAACCGCCAAAGTGGCCGCCTCCGACTGGACCTTTCTT
CACTGTCTCCCGCGGAAGCCTGAAGAAGTGGATGACGAAGTGTTTTACTCCCCTCG
GTCACTCGTGTTCCCGGAAGCAGAAAACAGGAAGTGGACCATTATGGCGGTCATGG
TGTCCCTCCTCACCGACTACAGCCCGCAGCTTCAGAAACCCAAGTTCTAG
>pARM726 (SEQ ID NO: 159)
ATGCTTTTCAATCTCCGCATCCTCCTTAACAACGCAGCGTTTAGAAACGGTCACAAC
TTCATGGTCCGGAACTTCCGCTGTGGACAGCCGCTTCAAAACAAGGTCCAGCTGAA
GGGTCGGGACCTTCTGACCCTGAAGAACTTTACTGGAGAAGAGATCAAGTACATGC
TTTGGCTGTCCGCGGACTTGAAGTTCCGCATTAAGCAGAAGGGAGAATACCTTCCG
CTGCTCCAAGGAAAGAGCCTGGGAATGATCTTTGAGAAGCGCTCAACCAGGACCCG
CCTTTCTACTGAAACTGGATTCGCGCTGCTGGGTGGTCACCCTTGCTTCCTGACGAC
CCAGGACATTCACCTCGGAGTGAACGAGTCCCTCACTGATACCGCCAGAGTGTTAT
CGAGCATGGCAGATGCCGTGCTGGCTAGGGTGTACAAACAGTCCGATCTGGACACC
CTGGCCAAGGAGGCATCAATTCCTATTATCAACGGACTTAGTGACCTCTACCATCCG
ATTCAAATCCTGGCCGATTACCTCACCCTGCAAGAACACTACAGCTCCCTGAAGGGT
CTGACATTGTCCTGGATCGGAGATGGAAACAACATTCTCCATTCCATCATGATGTCC
GCGGCCAAGTTCGGAATGCATCTCCAAGCCGCCACGCCGAAAGGATACGAGCCGGA
CGCTTCCGTGACTAAGCTCGCCGAGCAGTACGCTAAGGAGAACGGAACCAAGCTTC
TGCTGACTAACGACCCGCTAGAAGCCGCCCACGGTGGAAACGTGCTTATTACTGAC
ACCTGGATCTCCATGGGACAGGAAGAAGAGAAAAAGAAGCGGCTGCAGGCGTTCC
AGGGATATCAGGTCACCATGAAAACCGCCAAGGTCGCCGCCTCCGACTGGACCTTC
CTTCACTGCCTGCCTCGGAAGCCTGAAGAAGTGGACGACGAGGTGTTCTACTCGCC
GCGGAGCCTCGTGTTCCCTGAGGCCGAGAATAGAAAGTGGACCATCATGGCCGTGA
TGGTGTCACTCCTCACCGACTACAGCCCGCAGCTTCAGAAGCCTAAGTTCTAG
>pARM727 (SEQ ID NO: 160)
ATGCTTTTCAATCTCCGCATTCTCCTCAACAACGCAGCCTTTAGAAACGGCCACAAC
TTCATGGTCCGGAACTTCAGATGTGGCCAGCCGCTTCAGAACAAGGTCCAGCTCAA
GGGCCGGGACCTCCTCACCCTCAAAAACTTTACCGGCGAAGAGATCAAGTACATGC
TCTGGCTTTCGGCCGACCTTAAGTTCCGCATCAAGCAGAAGGGGGAATACCTTCCGC
TGCTTCAAGGAAAGTCCCTCGGCATGATCTTTGAAAAGCGCTCGACCAGGACCCGC
CTTTCCACTGAAACCGGGTTCGCGCTTCTCGGTGGCCACCCCTGCTTCCTCACCACC
CAAGACATTCACCTCGGAGTGAACGAATCCCTTACCGATACCGCAAGAGTGCTTTC
GTCGATGGCCGATGCCGTGCTTGCGCGGGTGTACAAGCAGTCAGATCTCGACACTC
TCGCCAAGGAGGCGTCCATTCCTATTATCAACGGCCTTTCCGACCTTTACCACCCGA
TTCAGATCCTCGCCGATTACCTCACCCTGCAAGAGCACTACTCGTCACTCAAGGGTC
TTACCCTCTCCTGGATCGGCGACGGAAACAACATCCTCCATTCGATCATGATGTCCG
CCGCCAAATTCGGCATGCACCTCCAAGCCGCGACCCCGAAGGGTTACGAGCCCGAC
GCTTCCGTGACCAAGCTCGCCGAACAGTACGCTAAGGAAAACGGCACCAAGCTCCT
CCTCACTAACGACCCTCTCGAAGCAGCCCATGGGGGCAACGTGCTCATTACTGACA
CTTGGATCTCGATGGGCCAGGAAGAGGAGAAAAAGAAGCGGCTTCAGGCGTTCCA
GGGATATCAGGTCACCATGAAAACCGCCAAGGTCGCTGCCTCGGACTGGACCTTCC
TTCACTGCCTTCCGCGCAAGCCTGAAGAGGTGGACGATGAGGTGTTCTACTCCCCAC
GGTCCCTTGTGTTCCCCGAGGCCGAGAATAGGAAGTGGACCATCATGGCCGTGATG
GTGTCGCTCCTCACTGACTACTCCCCGCAACTTCAGAAGCCTAAGTTCTAG
>pARM728 (SEQ ID NO: 161)
ATGCTGTTTAATCTGAGAATACTTCTAAACAACGCCGCCTTCCGGAATGGCCATAAC
TTTATGGTTCGGAATTTCCGCTGCGGCCAGCCGCTGCAGAACAAGGTCCAGCTGAA
GGGAAGAGACTTGCTGACCCTCAAGAACTTCACCGGAGAAGAAATCAAGTATATGC
TGTGGCTGTCCGCCGACCTGAAATTCCGCATCAAGCAGAAGGGCGAATATCTGCCG
CTGTTGCAAGGGAAGTCCCTGGGGATGATCTTCGAGAAGAGGTCCACCAGAACACG
GCTTTCAACCGAAACCGGGTTTGCACTGCTGGGTGGACACCCCTGTTTTCTGACCAC
TCAAGATATCCACCTGGGCGTGAACGAGTCCCTTACCGACACTGCTAGGGTGTTGTC
CAGCATGGCCGATGCCGTCCTGGCTCGCGTGTACAAGCAGTCCGACCTGGATACCC
TGGCAAAGGAAGCGTCCATTCCCATTATCAACGGGCTGTCCGACCTGTACCATCCG
ATTCAAATCCTGGCGGACTACCTGACTCTGCAAGAGCATTACAGCAGCTTGAAGGG
GCTTACTCTCTCGTGGATCGGCGACGGGAACAACATCCTGCACTCCATCATGATGTC
CGCCGCCAAGTTCGGGATGCATTTGCAAGCTGCGACCCCGAAAGGTTACGAGCCCG
ATGCTAGCGTAACTAAGCTTGCCGAACAGTACGCCAAAGAGAATGGTACAAAACTG
CTTCTGACTAACGACCCGCTGGAAGCAGCCCACGGCGGGAACGTGCTGATAACCGA
CACCTGGATTTCAATGGGGCAGGAGGAAGAGAAGAAGAAGCGACTGCAGGCGTTC
CAAGGCTATCAGGTTACCATGAAAACCGCCAAAGTGGCAGCCAGCGATTGGACTTT
CCTGCACTGTCTGCCGCGGAAGCCCGAGGAAGTTGATGACGAAGTATTCTACTCAC
CCCGGAGCCTCGTGTTCCCCGAGGCCGAAAACCGGAAGTGGACTATTATGGCCGTG
ATGGTGTCGCTGTTGACCGACTACAGCCCGCAACTGCAGAAGCCGAAGTTTTAG
>pARM729 (SEQ ID NO: 162)
ATGCTTTTCAACCTGAGGATCCTTTTGAACAACGCCGCCTTTCGCAACGGCCACAAC
TTTATGGTCCGCAATTTCCGCTGCGGGCAGCCGCTGCAGAACAAGGTCCAGCTGAA
GGGCCGGGATCTGCTGACCCTGAAGAACTTCACCGGGGAGGAAATCAAGTACATGC
TTTGGCTCTCCGCCGATCTGAAGTTCAGAATCAAGCAGAAGGGAGAGTACCTCCCG
TTGCTGCAAGGAAAGTCACTCGGAATGATTTTCGAAAAGAGAAGCACTAGGACCCG
CCTCTCAACTGAAACCGGGTTCGCGCTGCTCGGGGGCCATCCGTGTTTCCTGACTAC
CCAAGACATCCACCTGGGAGTGAACGAGTCGCTGACCGACACCGCACGCGTGCTGT
CATCCATGGCGGACGCAGTGCTTGCCCGGGTGTACAAGCAGTCGGACCTGGACACT
CTTGCCAAGGAGGCATCAATCCCCATCATTAACGGACTGTCCGATCTCTACCACCCG
ATTCAGATCCTGGCTGACTACCTAACCCTGCAAGAGCACTACTCAAGCCTGAAGGG
GCTGACCCTGTCGTGGATCGGGGACGGCAACAACATTCTGCACTCCATCATGATGTC
GGCGGCTAAGTTCGGGATGCATTTGCAAGCGGCAACTCCGAAGGGTTATGAACCCG
ACGCCTCCGTGACCAAGCTGGCCGAACAGTACGCCAAGGAAAACGGAACCAAGTT
GCTGCTGACTAATGATCCCCTGGAGGCGGCCCACGGGGGGAACGTGCTGATAACCG
ATACCTGGATCTCCATGGGGCAGGAAGAAGAGAAGAAAAAGCGGCTGCAGGCATT
CCAGGGATACCAGGTCACCATGAAAACCGCAAAAGTGGCAGCCAGCGACTGGACTT
TCCTCCATTGCCTGCCGCGAAAGCCGGAGGAGGTCGATGACGAGGTGTTCTACTCC
CCGCGGTCGCTGGTGTTCCCGGAGGCGGAAAACCGGAAGTGGACCATTATGGCCGT
GATGGTGTCACTCCTGACTGACTACAGCCCGCAACTGCAGAAGCCGAAGTTCTAG
>pARM1787 (SEQ ID NO: 163)
ATGCTTTTCAATCTCCGCATCCTCCTTAACAACGCCGCGTTTAGAAACGGCCACAAC
TTCATGGTCCGGAACTTCAGATGTGGCCAGCCGCTTCAAAACAAGGTCCAGCTGAA
GGGCCGGGATCTTCTGACCCTGAAGAACTTTACTGGCGAAGAGATCAAGTACATGC
TCTGGCTCTCCGCGGACTTGAAGTTCCGCATTAAGCAGAAGGGGGAATACCTTCCG
CTGCTTCAAGGAAAGAGCCTCGGCATGATCTTTGAGAAGCGCTCAACCAGGACCCG
CCTTTCTACTGAAACTGGGTTCGCGCTGCTCGGTGGCCACCCCTGCTTCCTGACGAC
CCAGGACATCCACCTCGGAGTGAACGAATCCCTCACCGATACCGCCCGGGTGTTAT
CGAGCATGGCAGATGCCGTGCTGGCCAGGGTGTACAAACAGTCCGATCTGGACACT
CTGGCCAAGGAGGCGTCAATTCCTATTATCAACGGCCTTAGTGACCTCTACCATCCG
ATTCAGATCCTGGCCGATTACCTCACCCTGCAAGAACACTACAGCTCCCTGAAGGGT
CTGACATTGTCCTGGATCGGCGACGGCAACAACATTCTCCATTCCATCATGATGTCC
GCCGCAAAATTCGGCATGCATCTTCAAGCCGCCACGCCGAAGGGTTACGAGCCCGA
CGCTTCCGTGACTAAGCTCGCCGAGCAGTACGCTAAGGAGAACGGAACCAAGCTTC
TGCTGACTAACGACCCACTAGAAGCAGCCCACGGGGGCAACGTGCTTATTACTGAC
ACCTGGATCTCCATGGGCCAGGAAGAAGAGAAAAAGAAGCGGCTGCAGGCGTTCC
AGGGATATCAGGTCACCATGAAAACCGCCAAGGTCGCTGCCTCCGACTGGACCTTC
CTGCACTGCCTGCCTCGCAAGCCTGAAGAAGTGGACGACGAGGTGTTCTACTCGCC
ACGGAGCCTCGTGTTCCCCGAGGCCGAGAATAGAAAGTGGACCATCATGGCCGTGA
TGGTGTCACTGCTCACCGACTACAGCCCGCAGCTTCAGAAGCCCAAGTTCTAGATA
AGTGAA
>pARM1788 (SEQ ID NO: 164)
ATGCTGTTCAACCTGCGCATCCTGCTGAACAACGCCGCCTTCCGCAACGGCCACAAC
TTCATGGTGCGCAACTTCCGCTGCGGCCAGCCCCTGCAGAACAAGGTGCAGCTGAA
GGGCCGCGACCTGCTGACCCTGAAGAACTTCACCGGCGAGGAGATCAAGTACATGC
TGTGGCTGAGCGCCGACCTGAAGTTCCGCATCAAGCAGAAGGGCGAGTACCTGCCC
CTGCTGCAGGGCAAGAGCCTGGGCATGATCTTCGAGAAGCGCAGCACCCGCACCCG
CCTGAGCACCGAGACAGGCCTGGCCCTGCTGGGCGGCCACCCCTGCTTCCTGACCA
CCCAGGACATCCACCTGGGCGTGAACGAGAGCCTGACCGACACCGCCCGCGTGCTG
AGCAGCATGGCCGACGCCGTGCTGGCCCGCGTGTACAAGCAGAGCGACCTGGACAC
CCTGGCCAAGGAGGCCAGCATCCCCATCATCAACGGCCTGAGCGACCTGTACCACC
CCATCCAGATCCTGGCCGACTACCTGACCCTGCAGGAGCACTACAGCAGCCTGAAG
GGCCTGACCCTGAGCTGGATCGGCGACGGCAACAACATCCTGCACAGCATCATGAT
GAGCGCCGCCAAGTTCGGCATGCACCTGCAGGCCGCCACCCCCAAGGGCTACGAGC
CCGACGCCAGCGTGACCAAGCTGGCCGAGCAGTACGCCAAGGAGAACGGCACCAA
GCTGCTGCTGACCAACGACCCCCTGGAGGCCGCCCACGGCGGCAACGTGCTGATCA
CCGACACCTGGATCAGCATGGGCCAGGAGGAGGAGAAGAAGAAGCGCCTGCAGGC
CTTCCAGGGCTACCAGGTGACCATGAAGACCGCCAAGGTGGCCGCCAGCGACTGGA
CCTTCCTGCACTGCCTGCCCCGCAAGCCCGAGGAGGTGGACGACGAGGTGTTCTAC
AGCCCCCGCAGCCTGGTGTTCCCCGAGGCCGAGAACCGCAAGTGGACCATCATGGC
CGTGATGGTGAGCCTGCTGACCGACTACAGCCCCCAGCTGCAGAAGCCCAAGTTCT
GAATAAGTGA
>pARM1789 (SEQ ID NO: 165)
ATGCTGTTCAACCTGCGCATCCTGCTGAACAACGCCGCCTTCCGCAACGGCCACAAC
TTCATGGTGCGCAACTTCCGCTGCGGCCAGCCCCTGCAGAACAAGGTGCAGCTGAA
GGGCCGCGACCTGCTGACCCTGAAGAACTTCACCGGCGAGGAGATCAAGTACATGC
TGTGGCTGAGCGCCGACCTGAAGTTCCGCATCAAGCAGAAGGGCGAGTACCTGCCC
CTGCTGCAGGGCAAGAGCCTGGGCATGATCTTCGAGAAGCGCAGCACCCGCACCCG
CCTGAGCACCGAGACAGGCCTGGCCCTGCTGGGCGGCCACCCCTGCTTCCTGACCA
CCCAGGACATCCACCTGGGCGTGAACGAGAGCCTGACCGACACCGCCCGCGTGCTG
AGCAGCATGGCCGACGCCGTGCTGGCCCGCGTGTACAAGCAGAGCGACCTGGACAC
CCTGGCCAAGGAGGCCAGCATCCCCATCATCAACGGCCTGAGCGACCTGTACCACC
CCATCCAGATCCTGGCCGACTACCTGACCCTGCAGGAGCACTACAGCAGCCTGAAG
GGCCTGACCCTGAGCTGGATCGGCGACGGCAACAACATCCTGCACAGCATCATGAT
GAGCGCCGCCAAGTTCGGCATGCACCTGCAGGCCGCCACCCCCAAGGGCTACGAGC
CCGACGCCAGCGTGACCAAGCTGGCCGAGCAGTACGCCAAGGAGAACGGCACCAA
GCTGCTGCTGACCAACGACCCCCTGGAGGCCGCCCACGGCGGCAACGTGCTGATCA
CCGACACCTGGATCAGCATGGGCCAGGAGGAGGAGAAGAAGAAGCGCCTGCAGGC
CTTCCAGGGCTACCAGGTGACCATGAAGACCGCCAAGGTGGCCGCCAGCGACTGGA
CCTTCCTGCACTGCCTGCCCCGCAAGCCCGAGGAGGTGGACGACGAGGTGTTCTAC
AGCCCCCGCAGCCTGGTGTTCCCCGAGGCCGAGAACCGCAAGTGGACCATCATGGC
CGTGATGGTGAGCCTGCTGACCGACTACAGCCCCCAGCTGCAGAAGCCCAAGTTCT
GAATAAGTGA
>pARM1790 (SEQ ID NO: 166)
ATGCTTTTCAATCTCCGCATCCTCCTTAACAACGCCGCGTTTAGAAACGGCCACAAC
TTCATGGTCCGGAACTTCAGATGTGGCCAGCCGCTTCAAAACAAGGTCCAGCTGAA
GGGCCGGGATCTTCTGACCCTGAAGAACTTTACTGGCGAAGAGATCAAGTACATGC
TCTGGCTCTCCGCGGACTTGAAGTTCCGCATTAAGCAGAAGGGGGAATACCTTCCG
CTGCTTCAAGGAAAGAGCCTCGGCATGATCTTTGAGAAGCGCTCAACCAGGACCCG
CCTTTCTACTGAAACTGGGTTCGCGCTGCTCGGTGGCCACCCCTGCTTCCTGACGAC
CCAGGACATCCACCTCGGAGTGAACGAATCCCTCACCGATACCGCCCGGGTGTTAT
CGAGCATGGCAGATGCCGTGCTGGCCAGGGTGTACAAACAGTCCGATCTGGACACT
CTGGCCAAGGAGGCGTCAATTCCTATTATCAACGGCCTTAGTGACCTCTACCATCCG
ATTCAGATCCTGGCCGATTACCTCACCCTGCAAGAACACTACAGCTCCCTGAAGGGT
CTGACATTGTCCTGGATCGGCGACGGCAACAACATTCTCCATTCCATCATGATGTCC
GCCGCAAAATTCGGCATGCATCTTCAAGCCGCCACGCCGAAGGGTTACGAGCCCGA
CGCTTCCGTGACTAAGCTCGCCGAGCAGTACGCTAAGGAGAACGGAACCAAGCTTC
TGCTGACTAACGACCCACTAGAAGCAGCCCACGGGGGCAACGTGCTTATTACTGAC
ACCTGGATCTCCATGGGCCAGGAAGAAGAGAAAAAGAAGCGGCTGCAGGCGTTCC
AGGGATATCAGGTCACCATGAAAACCGCCAAGGTCGCTGCCTCCGACTGGACCTTC
CTGCACTGCCTGCCTCGCAAGCCTGAAGAAGTGGACGACGAGGTGTTCTACTCGCC
ACGGAGCCTCGTGTTCCCCGAGGCCGAGAATAGAAAGTGGACCATCATGGCCGTGA
TGGTGTCACTGCTCACCGACTACAGCCCGCAGCTTCAGAAGCCCAAGTTCTAGATA
AGTGAA
>pARM1791 (SEQ ID NO: 167)
ATGCTTTTCAACCTGAGAATCCTCTTGAACAATGCTGCTTTTCGGAATGGCCACAAC
TTTATGGTTCGGAACTTCCGTTGCGGCCAGCCTTTACAAAACAAGGTCCAGCTGAAG
GGCCGGGATTTGCTCACACTAAAGAACTTTACTGGAGAAGAGATCAAGTACATGCT
ATGGCTGTCGGCCGACCTGAAGTTCCGTATCAAGCAGAAGGGAGAATACCTTCCGC
TGCTTCAAGGAAAGAGCCTCGGCATGATCTTTGAGAAGCGCTCAACCAGGACCCGC
CTTTCTACTGAAACTGGGTTCGCGCTGCTCGGTGGCCACCCCTGCTTCCTGACGACC
CAGGACATCCACCTCGGAGTGAACGAATCCCTCACCGATACCGCCCGGGTGTTATC
GAGCATGGCAGATGCCGTGCTGGCCAGGGTGTACAAACAGTCCGATCTCGATACCT
TGGCAAAGGAGGCTTCCATTCCCATCATCAACGGCCTGAGCGACCTGTACCACCCA
ATCCAAATCCTGGCTGACTACCTGACCCTGCAAGAGCACTACAGCAGCCTGAAGGG
TCTGACCCTGTCATGGATTGGCGATGGAAACAATATTCTGCACTCCATCATGATGTC
CGCCGCGAAGTTCGGAATGCATCTGCAAGCCGCCACTCCAAAAGGATACGAACCGG
ATGCATCCGTGACCAAGTTGGCGGAACAGTACGCGAAGGAGAACGGAACCAAGCT
CCTGCTGACTAACGACCCGCTCGAGGCTGCGCATGGGGGTAACGTGCTGATTACGG
ACACCTGGATCTCCATGGGGCAGGAGGAAGAGAAGAAGAAGAGACTGCAGGCATT
CCAGGGGTACCAGGTCACCATGAAAACCGCAAAAGTGGCAGCTTCGGACTGGACTT
TCCTGCATTGCCTGCCGAGGAAGCCGGAGGAAGTCGACGACGAAGTGTTCTACTCG
CCTCGGTCCCTGGTGTTCCCCGAGGCCGAAAACCGGAAGTGGACCATCATGGCCGT
GATGGTGTCCTTGCTGACTGACTATAGCCCGCAGCTGCAGAAGCCTAAGTTCTAGAT
AAGTGA
>pARM1792 (SEQ ID NO: 168)
ATGCTGTTCAACCTGCGCATCCTGCTGAACAACGCCGCCTTCCGCAACGGCCACAAC
TTCATGGTGCGCAACTTCCGCTGCGGCCAGCCCCTGCAGAACAAGGTGCAGCTGAA
GGGCCGCGACCTGCTGACCCTGAAGAACTTCACCGGCGAGGAGATCAAGTACATGC
TGTGGCTGAGCGCCGACCTGAAGTTCCGCATCAAGCAGAAGGGCGAGTACCTGCCC
CTGCTGCAGGGCAAGAGCCTGGGCATGATCTTCGAGAAGCGCAGCACCCGCACCCG
CCTGAGCACCGAGACAGGCCTGGCCCTGCTGGGCGGCCACCCCTGCTTCCTGACCA
CCCAGGACATCCACCTGGGCGTGAACGAGAGCCTGACCGACACCGCCCGCGTGCTG
AGCAGCATGGCCGACGCCGTGCTGGCCCGCGTGTACAAGCAGAGCGACCTGGACAC
CCTGGCCAAGGAGGCCAGCATCCCCATCATCAACGGCCTGAGCGACCTGTACCACC
CCATCCAGATCCTGGCCGACTACCTGACCCTGCAGGAGCACTACAGCAGCCTGAAG
GGCCTGACCCTGAGCTGGATCGGCGACGGCAACAACATCCTGCACAGCATCATGAT
GAGCGCCGCCAAGTTCGGCATGCACCTGCAGGCCGCCACCCCCAAGGGCTACGAGC
CCGACGCCAGCGTGACCAAGCTGGCCGAGCAGTACGCCAAGGAGAACGGCACCAA
GCTGCTGCTGACCAACGACCCCCTGGAGGCCGCCCACGGCGGCAACGTGCTGATCA
CCGACACCTGGATCAGCATGGGCCAGGAGGAGGAGAAGAAGAAGCGCCTGCAGGC
CTTCCAGGGCTACCAGGTGACCATGAAGACCGCCAAGGTGGCCGCCAGCGACTGGA
CCTTCCTGCACTGCCTGCCCCGCAAGCCCGAGGAGGTGGACGACGAGGTGTTCTAC
AGCCCCCGCAGCCTGGTGTTCCCCGAGGCCGAGAACCGCAAGTGGACCATCATGGC
CGTGATGGTGAGCCTGCTGACCGACTACAGCCCCCAGCTGCAGAAGCCCAAGTTCT
GAATAAGTGA
>pARM1793 (SEQ ID NO: 169)
ATGCTTTTCAATCTCCGCATCCTCCTTAACAACGCCGCGTTTAGAAACGGCCACAAC
TTCATGGTCCGGAACTTCAGATGTGGCCAGCCGCTTCAAAACAAGGTCCAGCTGAA
GGGCCGGGATCTTCTGACCCTGAAGAACTTTACTGGCGAAGAGATCAAGTACATGC
TCTGGCTCTCCGCGGACTTGAAGTTCCGCATTAAGCAGAAGGGGGAATACCTTCCG
CTGCTTCAAGGAAAGAGCCTCGGCATGATCTTTGAGAAGCGCTCAACCAGGACCCG
CCTTTCTACTGAAACTGGGTTCGCGCTGCTCGGTGGCCACCCCTGCTTCCTGACGAC
CCAGGACATCCACCTCGGAGTGAACGAATCCCTCACCGATACCGCCCGGGTGTTAT
CGAGCATGGCAGATGCCGTGCTGGCCAGGGTGTACAAACAGTCCGATCTGGACACT
CTGGCCAAGGAGGCGTCAATTCCTATTATCAACGGCCTTAGTGACCTCTACCATCCG
ATTCAGATCCTGGCCGATTACCTCACCCTGCAAGAACACTACAGCTCCCTGAAGGGT
CTGACATTGTCCTGGATCGGCGACGGCAACAACATTCTCCATTCCATCATGATGTCC
GCCGCAAAATTCGGCATGCATCTTCAAGCCGCCACGCCGAAGGGTTACGAGCCCGA
CGCTTCCGTGACTAAGCTCGCCGAGCAGTACGCTAAGGAGAACGGAACCAAGCTTC
TGCTGACTAACGACCCACTAGAAGCAGCCCACGGGGGCAACGTGCTTATTACTGAC
ACCTGGATCTCCATGGGCCAGGAAGAAGAGAAAAAGAAGCGGCTGCAGGCGTTCC
AGGGATATCAGGTCACCATGAAAACCGCCAAGGTCGCTGCCTCCGACTGGACCTTC
CTGCACTGCCTGCCTCGCAAGCCTGAAGAAGTGGACGACGAGGTGTTCTACTCGCC
ACGGAGCCTCGTGTTCCCCGAGGCCGAGAATAGAAAGTGGACCATCATGGCCGTGA
TGGTGTCACTGCTCACCGACTACAGCCCGCAGCTTCAGAAGCCCAAGTTCTAGATA
AGTGAA
>pARM1794 (SEQ ID NO: 170)
ATGCTGTTCAACCTGCGCATCCTGCTGAACAACGCCGCCTTCCGCAACGGCCACAAC
TTCATGGTGCGCAACTTCCGCTGCGGCCAGCCCCTGCAGAACAAGGTGCAGCTGAA
GGGCCGCGACCTGCTGACCCTGAAGAACTTCACCGGCGAGGAGATCAAGTACATGC
TGTGGCTGAGCGCCGACCTGAAGTTCCGCATCAAGCAGAAGGGCGAGTACCTGCCC
CTGCTGCAGGGCAAGAGCCTGGGCATGATCTTCGAGAAGCGCAGCACCCGCACCCG
CCTGAGCACCGAGACAGGCCTGGCCCTGCTGGGCGGCCACCCCTGCTTCCTGACCA
CCCAGGACATCCACCTGGGCGTGAACGAGAGCCTGACCGACACCGCCCGCGTGCTG
AGCAGCATGGCCGACGCCGTGCTGGCCCGCGTGTACAAGCAGAGCGACCTGGACAC
CCTGGCCAAGGAGGCCAGCATCCCCATCATCAACGGCCTGAGCGACCTGTACCACC
CCATCCAGATCCTGGCCGACTACCTGACCCTGCAGGAGCACTACAGCAGCCTGAAG
GGCCTGACCCTGAGCTGGATCGGCGACGGCAACAACATCCTGCACAGCATCATGAT
GAGCGCCGCCAAGTTCGGCATGCACCTGCAGGCCGCCACCCCCAAGGGCTACGAGC
CCGACGCCAGCGTGACCAAGCTGGCCGAGCAGTACGCCAAGGAGAACGGCACCAA
GCTGCTGCTGACCAACGACCCCCTGGAGGCCGCCCACGGCGGCAACGTGCTGATCA
CCGACACCTGGATCAGCATGGGCCAGGAGGAGGAGAAGAAGAAGCGCCTGCAGGC
CTTCCAGGGCTACCAGGTGACCATGAAGACCGCCAAGGTGGCCGCCAGCGACTGGA
CCTTCCTGCACTGCCTGCCCCGCAAGCCCGAGGAGGTGGACGACGAGGTGTTCTAC
AGCCCCCGCAGCCTGGTGTTCCCCGAGGCCGAGAACCGCAAGTGGACCATCATGGC
CGTGATGGTGAGCCTGCTGACCGACTACAGCCCCCAGCTGCAGAAGCCCAAGTTCT
GAATAAGTGA
>pARM1795 (SEQ ID NO: 171)
ATGCTTTTCAACCTGAGAATCCTCTTGAACAATGCTGCTTTTCGGAATGGCCACAAC
TTTATGGTTCGGAACTTCCGTTGCGGCCAGCCTTTACAAAACAAGGTCCAGCTGAAG
GGCCGGGATTTGCTCACACTAAAGAACTTTACTGGAGAAGAGATCAAGTACATGCT
ATGGCTGTCGGCCGACCTGAAGTTCCGTATCAAGCAGAAGGGAGAATACCTTCCGC
TGCTTCAAGGAAAGAGCCTCGGCATGATCTTTGAGAAGCGCTCAACCAGGACCCGC
CTTTCTACTGAAACTGGGTTCGCGCTGCTCGGTGGCCACCCCTGCTTCCTGACGACC
CAGGACATCCACCTCGGAGTGAACGAATCCCTCACCGATACCGCCCGGGTGTTATC
GAGCATGGCAGATGCCGTGCTGGCCAGGGTGTACAAACAGTCCGATCTCGATACCT
TGGCAAAGGAGGCTTCCATTCCCATCATCAACGGCCTGAGCGACCTGTACCACCCA
ATCCAAATCCTGGCTGACTACCTGACCCTGCAAGAGCACTACAGCAGCCTGAAGGG
TCTGACCCTGTCATGGATTGGCGATGGAAACAATATTCTGCACTCCATCATGATGTC
CGCCGCGAAGTTCGGAATGCATCTGCAAGCCGCCACTCCAAAAGGATACGAACCGG
ATGCATCCGTGACCAAGTTGGCGGAACAGTACGCGAAGGAGAACGGAACCAAGCT
CCTGCTGACTAACGACCCGCTCGAGGCTGCGCATGGGGGTAACGTGCTGATTACGG
ACACCTGGATCTCCATGGGGCAGGAGGAAGAGAAGAAGAAGAGACTGCAGGCATT
CCAGGGGTACCAGGTCACCATGAAAACCGCAAAAGTGGCAGCTTCGGACTGGACTT
TCCTGCATTGCCTGCCGAGGAAGCCGGAGGAAGTCGACGACGAAGTGTTCTACTCG
CCTCGGTCCCTGGTGTTCCCCGAGGCCGAAAACCGGAAGTGGACCATCATGGCCGT
GATGGTGTCCTTGCTGACTGACTATAGCCCGCAGCTGCAGAAGCCTAAGTTCTAGAT
AAGTGA
>pARM1796 (SEQ ID NO: 172)
ATGCTTTTCAATCTCCGCATCCTCCTTAACAACGCCGCGTTTAGAAACGGCCACAAC
TTCATGGTCCGGAACTTCAGATGTGGCCAGCCGCTTCAAAACAAGGTCCAGCTGAA
GGGCCGGGATCTTCTGACCCTGAAGAACTTTACTGGCGAAGAGATCAAGTACATGC
TCTGGCTCTCCGCGGACTTGAAGTTCCGCATTAAGCAGAAGGGGGAATACCTTCCG
CTGCTTCAAGGAAAGAGCCTCGGCATGATCTTTGAGAAGCGCTCAACCAGGACCCG
CCTTTCTACTGAAACTGGGTTCGCGCTGCTCGGTGGCCACCCCTGCTTCCTGACGAC
CCAGGACATCCACCTCGGAGTGAACGAATCCCTCACCGATACCGCCCGGGTGTTAT
CGAGCATGGCAGATGCCGTGCTGGCCAGGGTGTACAAACAGTCCGATCTGGACACT
CTGGCCAAGGAGGCGTCAATTCCTATTATCAACGGCCTTAGTGACCTCTACCATCCG
ATTCAGATCCTGGCCGATTACCTCACCCTGCAAGAACACTACAGCTCCCTGAAGGGT
CTGACATTGTCCTGGATCGGCGACGGCAACAACATTCTCCATTCCATCATGATGTCC
GCCGCAAAATTCGGCATGCATCTTCAAGCCGCCACGCCGAAGGGTTACGAGCCCGA
CGCTTCCGTGACTAAGCTCGCCGAGCAGTACGCTAAGGAGAACGGAACCAAGCTTC
TGCTGACTAACGACCCACTAGAAGCAGCCCACGGGGGCAACGTGCTTATTACTGAC
ACCTGGATCTCCATGGGCCAGGAAGAAGAGAAAAAGAAGCGGCTGCAGGCGTTCC
AGGGATATCAGGTCACCATGAAAACCGCCAAGGTCGCTGCCTCCGACTGGACCTTC
CTGCACTGCCTGCCTCGCAAGCCTGAAGAAGTGGACGACGAGGTGTTCTACTCGCC
ACGGAGCCTCGTGTTCCCCGAGGCCGAGAATAGAAAGTGGACCATCATGGCCGTGA
TGGTGTCACTGCTCACCGACTACAGCCCGCAGCTTCAGAAGCCCAAGTTCTAGATA
AGTGAA
>pARM1797 (SEQ ID NO: 173)
ATGCTGTTCAACCTGCGCATCCTGCTGAACAACGCCGCCTTCCGCAACGGCCACAAC
TTCATGGTGCGCAACTTCCGCTGCGGCCAGCCCCTGCAGAACAAGGTGCAGCTGAA
GGGCCGCGACCTGCTGACCCTGAAGAACTTCACCGGCGAGGAGATCAAGTACATGC
TGTGGCTGAGCGCCGACCTGAAGTTCCGCATCAAGCAGAAGGGCGAGTACCTGCCC
CTGCTGCAGGGCAAGAGCCTGGGCATGATCTTCGAGAAGCGCAGCACCCGCACCCG
CCTGAGCACCGAGACAGGCCTGGCCCTGCTGGGCGGCCACCCCTGCTTCCTGACCA
CCCAGGACATCCACCTGGGCGTGAACGAGAGCCTGACCGACACCGCCCGCGTGCTG
AGCAGCATGGCCGACGCCGTGCTGGCCCGCGTGTACAAGCAGAGCGACCTGGACAC
CCTGGCCAAGGAGGCCAGCATCCCCATCATCAACGGCCTGAGCGACCTGTACCACC
CCATCCAGATCCTGGCCGACTACCTGACCCTGCAGGAGCACTACAGCAGCCTGAAG
GGCCTGACCCTGAGCTGGATCGGCGACGGCAACAACATCCTGCACAGCATCATGAT
GAGCGCCGCCAAGTTCGGCATGCACCTGCAGGCCGCCACCCCCAAGGGCTACGAGC
CCGACGCCAGCGTGACCAAGCTGGCCGAGCAGTACGCCAAGGAGAACGGCACCAA
GCTGCTGCTGACCAACGACCCCCTGGAGGCCGCCCACGGCGGCAACGTGCTGATCA
CCGACACCTGGATCAGCATGGGCCAGGAGGAGGAGAAGAAGAAGCGCCTGCAGGC
CTTCCAGGGCTACCAGGTGACCATGAAGACCGCCAAGGTGGCCGCCAGCGACTGGA
CCTTCCTGCACTGCCTGCCCCGCAAGCCCGAGGAGGTGGACGACGAGGTGTTCTAC
AGCCCCCGCAGCCTGGTGTTCCCCGAGGCCGAGAACCGCAAGTGGACCATCATGGC
CGTGATGGTGAGCCTGCTGACCGACTACAGCCCCCAGCTGCAGAAGCCCAAGTTCT
GAATAAGTGA
>pARM1798 (SEQ ID NO: 174)
ATGCTTTTCAACCTGAGAATCCTCTTGAACAATGCTGCTTTTCGGAATGGCCACAAC
TTTATGGTTCGGAACTTCCGTTGCGGCCAGCCTTTACAAAACAAGGTCCAGCTGAAG
GGCCGGGATTTGCTCACACTAAAGAACTTTACTGGAGAAGAGATCAAGTACATGCT
ATGGCTGTCGGCCGACCTGAAGTTCCGTATCAAGCAGAAGGGAGAATACCTTCCGC
TGCTTCAAGGAAAGAGCCTCGGCATGATCTTTGAGAAGCGCTCAACCAGGACCCGC
CTTTCTACTGAAACTGGGTTCGCGCTGCTCGGTGGCCACCCCTGCTTCCTGACGACC
CAGGACATCCACCTCGGAGTGAACGAATCCCTCACCGATACCGCCCGGGTGTTATC
GAGCATGGCAGATGCCGTGCTGGCCAGGGTGTACAAACAGTCCGATCTCGATACCT
TGGCAAAGGAGGCTTCCATTCCCATCATCAACGGCCTGAGCGACCTGTACCACCCA
ATCCAAATCCTGGCTGACTACCTGACCCTGCAAGAGCACTACAGCAGCCTGAAGGG
TCTGACCCTGTCATGGATTGGCGATGGAAACAATATTCTGCACTCCATCATGATGTC
CGCCGCGAAGTTCGGAATGCATCTGCAAGCCGCCACTCCAAAAGGATACGAACCGG
ATGCATCCGTGACCAAGTTGGCGGAACAGTACGCGAAGGAGAACGGAACCAAGCT
CCTGCTGACTAACGACCCGCTCGAGGCTGCGCATGGGGGTAACGTGCTGATTACGG
ACACCTGGATCTCCATGGGGCAGGAGGAAGAGAAGAAGAAGAGACTGCAGGCATT
CCAGGGGTACCAGGTCACCATGAAAACCGCAAAAGTGGCAGCTTCGGACTGGACTT
TCCTGCATTGCCTGCCGAGGAAGCCGGAGGAAGTCGACGACGAAGTGTTCTACTCG
CCTCGGTCCCTGGTGTTCCCCGAGGCCGAAAACCGGAAGTGGACCATCATGGCCGT
GATGGTGTCCTTGCTGACTGACTATAGCCCGCAGCTGCAGAAGCCTAAGTTCTAGAT
AAGTGA
>pARM1799 (SEQ ID NO: 175)
ATGCTGTTCAACCTGCGCATCCTGCTGAACAACGCCGCCTTCCGCAACGGCCACAAC
TTCATGGTGCGCAACTTCCGCTGCGGCCAGCCCCTGCAGAACAAGGTGCAGCTGAA
GGGCCGCGACCTGCTGACCCTGAAGAACTTCACCGGCGAGGAGATCAAGTACATGC
TGTGGCTGAGCGCCGACCTGAAGTTCCGCATCAAGCAGAAGGGCGAGTACCTGCCC
CTGCTGCAGGGCAAGAGCCTGGGCATGATCTTCGAGAAGCGCAGCACCCGCACCCG
CCTGAGCACCGAGACAGGCTTCGCCCTGCTGGGCGGCCACCCCTGCTTCCTGACCAC
CCAGGACATCCACCTGGGCGTGAACGAGAGCCTGACCGACACCGCCCGCGTGCTGA
GCAGCATGGCCGACGCCGTGCTGGCCCGCGTGTACAAGCAGAGCGACCTGGACACC
CTGGCCAAGGAGGCCAGCATCCCCATCATCAACGGCCTGAGCGACCTGTACCACCC
CATCCAGATCCTGGCCGACTACCTGACCCTGCAGGAGCACTACAGCAGCCTGAAGG
GCCTGACCCTGAGCTGGATCGGCGACGGCAACAACATCCTGCACAGCATCATGATG
AGCGCCGCCAAGTTCGGCATGCACCTGCAGGCCGCCACCCCCAAGGGCTACGAGCC
CGACGCCAGCGTGACCAAGCTGGCCGAGCAGTACGCCAAGGAGAACGGCACCAAG
CTGCTGCTGACCAACGACCCCCTGGAGGCCGCCCACGGCGGCAACGTGCTGATCAC
CGACACCTGGATCAGCATGGGCCAGGAGGAGGAGAAGAAGAAGCGCCTGCAGGCC
TTCCAGGGCTACCAGGTGACCATGAAGACCGCCAAGGTGGCCGCCAGCGACTGGAC
CTTCCTGCACTGCCTGCCCCGCAAGCCCGAGGAGGTGGACGACGAGGTGTTCTACA
GCCCCCGCAGCCTGGTGTTCCCCGAGGCCGAGAACCGCAAGTGGACCATCATGGCC
GTGATGGTGAGCCTGCTGACCGACTACAGCCCCCAGCTGCAGAAGCCCAAGTTCTG
A
>pARM1800 (SEQ ID NO: 176)
ATGCTGTTCAACCTGCGCATCCTGCTGAACAACGCCGCCTTCCGCAACGGCCACAAC
TTCATGGTGCGCAACTTCCGCTGCGGCCAGCCCCTGCAGAACAAGGTGCAGCTGAA
GGGCCGCGACCTGCTGACCCTGAAGAACTTCACCGGCGAGGAGATCAAGTACATGC
TGTGGCTGAGCGCCGACCTGAAGTTCCGCATCAAGCAGAAGGGCGAGTACCTGCCC
CTGCTGCAGGGCAAGAGCCTGGGCATGATCTTCGAGAAGCGCAGCACCCGCACCCG
CCTGAGCACCGAGACAGGCTTCGCCCTGCTGGGCGGCCACCCCTGCTTCCTGACCAC
CCAGGACATCCACCTGGGCGTGAACGAGAGCCTGACCGACACCGCCCGCGTGCTGA
GCAGCATGGCCGACGCCGTGCTGGCCCGCGTGTACAAGCAGAGCGACCTGGACACC
CTGGCCAAGGAGGCCAGCATCCCCATCATCAACGGCCTGAGCGACCTGTACCACCC
CATCCAGATCCTGGCCGACTACCTGACCCTGCAGGAGCACTACAGCAGCCTGAAGG
GCCTGACCCTGAGCTGGATCGGCGACGGCAACAACATCCTGCACAGCATCATGATG
AGCGCCGCCAAGTTCGGCATGCACCTGCAGGCCGCCACCCCCAAGGGCTACGAGCC
CGACGCCAGCGTGACCAAGCTGGCCGAGCAGTACGCCAAGGAGAACGGCACCAAG
CTGCTGCTGACCAACGACCCCCTGGAGGCCGCCCACGGCGGCAACGTGCTGATCAC
CGACACCTGGATCAGCATGGGCCAGGAGGAGGAGAAGAAGAAGCGCCTGCAGGCC
TTCCAGGGCTACCAGGTGACCATGAAGACCGCCAAGGTGGCCGCCAGCGACTGGAC
CTTCCTGCACTGCCTGCCCCGCAAGCCCGAGGAGGTGGACGACGAGGTGTTCTACA
GCCCCCGCAGCCTGGTGTTCCCCGAGGCCGAGAACCGCAAGTGGACCATCATGGCC
GTGATGGTGAGCCTGCTGACCGACTACAGCCCCCAGCTGCAGAAGCCCAAGTTCTG
A
>pARM1801 (SEQ ID NO: 177)
ATGCTGTTCAACCTGCGCATCCTGCTGAACAACGCCGCCTTCCGCAACGGCCACAAC
TTCATGGTGCGCAACTTCCGCTGCGGCCAGCCCCTGCAGAACAAGGTGCAGCTGAA
GGGCCGCGACCTGCTGACCCTGAAGAACTTCACCGGCGAGGAGATCAAGTACATGC
TGTGGCTGAGCGCCGACCTGAAGTTCCGCATCAAGCAGAAGGGCGAGTACCTGCCC
CTGCTGCAGGGCAAGAGCCTGGGCATGATCTTCGAGAAGCGCAGCACCCGCACCCG
CCTGAGCACCGAGACAGGCTTCGCCCTGCTGGGCGGCCACCCCTGCTTCCTGACCAC
CCAGGACATCCACCTGGGCGTGAACGAGAGCCTGACCGACACCGCCCGCGTGCTGA
GCAGCATGGCCGACGCCGTGCTGGCCCGCGTGTACAAGCAGAGCGACCTGGACACC
CTGGCCAAGGAGGCCAGCATCCCCATCATCAACGGCCTGAGCGACCTGTACCACCC
CATCCAGATCCTGGCCGACTACCTGACCCTGCAGGAGCACTACAGCAGCCTGAAGG
GCCTGACCCTGAGCTGGATCGGCGACGGCAACAACATCCTGCACAGCATCATGATG
AGCGCCGCCAAGTTCGGCATGCACCTGCAGGCCGCCACCCCCAAGGGCTACGAGCC
CGACGCCAGCGTGACCAAGCTGGCCGAGCAGTACGCCAAGGAGAACGGCACCAAG
CTGCTGCTGACCAACGACCCCCTGGAGGCCGCCCACGGCGGCAACGTGCTGATCAC
CGACACCTGGATCAGCATGGGCCAGGAGGAGGAGAAGAAGAAGCGCCTGCAGGCC
TTCCAGGGCTACCAGGTGACCATGAAGACCGCCAAGGTGGCCGCCAGCGACTGGAC
CTTCCTGCACTGCCTGCCCCGCAAGCCCGAGGAGGTGGACGACGAGGTGTTCTACA
GCCCCCGCAGCCTGGTGTTCCCCGAGGCCGAGAACCGCAAGTGGACCATCATGGCC
GTGATGGTGAGCCTGCTGACCGACTACAGCCCCCAGCTGCAGAAGCCCAAGTTCTG
A
>pARM1802 (SEQ ID NO: 178)
ATGCTGTTCAACCTGCGCATCCTGCTGAACAACGCCGCCTTCCGCAACGGCCACAAC
TTCATGGTGCGCAACTTCCGCTGCGGCCAGCCCCTGCAGAACAAGGTGCAGCTGAA
GGGCCGCGACCTGCTGACCCTGAAGAACTTCACCGGCGAGGAGATCAAGTACATGC
TGTGGCTGAGCGCCGACCTGAAGTTCCGCATCAAGCAGAAGGGCGAGTACCTGCCC
CTGCTGCAGGGCAAGAGCCTGGGCATGATCTTCGAGAAGCGCAGCACCCGCACCCG
CCTGAGCACCGAGACAGGCTTCGCCCTGCTGGGCGGCCACCCCTGCTTCCTGACCAC
CCAGGACATCCACCTGGGCGTGAACGAGAGCCTGACCGACACCGCCCGCGTGCTGA
GCAGCATGGCCGACGCCGTGCTGGCCCGCGTGTACAAGCAGAGCGACCTGGACACC
CTGGCCAAGGAGGCCAGCATCCCCATCATCAACGGCCTGAGCGACCTGTACCACCC
CATCCAGATCCTGGCCGACTACCTGACCCTGCAGGAGCACTACAGCAGCCTGAAGG
GCCTGACCCTGAGCTGGATCGGCGACGGCAACAACATCCTGCACAGCATCATGATG
AGCGCCGCCAAGTTCGGCATGCACCTGCAGGCCGCCACCCCCAAGGGCTACGAGCC
CGACGCCAGCGTGACCAAGCTGGCCGAGCAGTACGCCAAGGAGAACGGCACCAAG
CTGCTGCTGACCAACGACCCCCTGGAGGCCGCCCACGGCGGCAACGTGCTGATCAC
CGACACCTGGATCAGCATGGGCCAGGAGGAGGAGAAGAAGAAGCGCCTGCAGGCC
TTCCAGGGCTACCAGGTGACCATGAAGACCGCCAAGGTGGCCGCCAGCGACTGGAC
CTTCCTGCACTGCCTGCCCCGCAAGCCCGAGGAGGTGGACGACGAGGTGTTCTACA
GCCCCCGCAGCCTGGTGTTCCCCGAGGCCGAGAACCGCAAGTGGACCATCATGGCC
GTGATGGTGAGCCTGCTGACCGACTACAGCCCCCAGCTGCAGAAGCCCAAGTTCTG
AATAAGTGAA
>pARM1803 (SEQ ID NO: 179)
ATGGGCGTCTTCAACCTGCGGATCCTGCTGAACAACGCCGCCTTCCGGAACGGCCA
CAACTTCATGGTCCGCAACTTCAGATGCGGCCAGCCCCTGCAGAACAAGGTGCAGC
TGAAGGGCCGGGACCTGCTGACCCTGAAGAACTTCACCGGCGAAGAGATCAAGTAC
ATGCTGTGGCTGAGCGCCGACCTGAAGTTCCGGATCAAGCAGAAGGGCGAGTACCT
GCCCCTGCTGCAAGGCAAGAGCCTGGGCATGATCTTCGAGAAGCGGAGCACCCGGA
CCCGGCTGAGCACCGAGACAGGCTTTGCCCTGCTGGGAGGCCACCCCTGCTTTCTGA
CCACCCAGGACATCCACCTGGGCGTGAACGAGAGCCTGACCGACACCGCCAGAGTG
CTGAGCAGCATGGCCGACGCCGTGCTGGCCCGGGTGTACAAGCAGAGCGACCTGGA
CACCCTGGCCAAAGAGGCCAGCATCCCCATCATCAACGGCCTGAGCGACCTGTACC
ACCCCATCCAGATCCTGGCCGACTACCTGACCCTGCAGGAACACTACAGCTCCCTG
AAGGGCCTGACCCTGAGCTGGATCGGCGACGGCAACAACATCCTGCACAGCATCAT
GATGAGCGCCGCCAAGTTCGGCATGCATCTGCAGGCCGCCACCCCCAAGGGCTACG
AGCCTGATGCCAGCGTGACCAAGCTGGCCGAGCAGTACGCCAAAGAGAACGGCAC
CAAGCTGCTGCTGACCAACGACCCCCTGGAAGCCGCCCACGGCGGCAACGTGCTGA
TCACCGACACCTGGATCAGCATGGGCCAGGAAGAGGAAAAGAAGAAGCGGCTGCA
GGCCTTCCAGGGCTACCAGGTCACAATGAAGACCGCCAAGGTGGCCGCCAGCGACT
GGACCTTCCTGCACTGCCTGCCCCGGAAGCCCGAAGAGGTGGACGACGAGGTGTTC
TACAGCCCCCGGTCCCTGGTGTTCCCCGAGGCCGAGAACCGGAAGTGGACCATTAT
GGCCGTGATGGTGTCCCTGCTGACCGACTACTCCCCCCAGCTGCAGAAGCCCAAGTT
CTAGATAAGTGAA
>pARM1804 (SEQ ID NO: 180)
ATGGGCGTCTTCAACCTGCGGATCCTGCTGAACAACGCCGCCTTCCGGAACGGCCA
CAACTTCATGGTCCGCAACTTCAGATGCGGCCAGCCCCTGCAGAACAGGGTGCAGC
TGAAGGGCCGGGACCTGCTGACCCTGAAGAACTTCACCGGCGAAGAGATCAGGTAC
ATGCTGTGGCTGAGCGCCGACCTGAAGTTCCGGATCAAGCAGAAGGGCGAGTACCT
GCCCCTGCTGCAAGGCAAGAGCCTGGGCATGATCTTCGAGAAGCGGAGCACCCGGA
CCCGGCTGAGCACCGAGACAGGCTTTGCCCTGCTGGGAGGCCACCCCTGCTTTCTGA
CCACCCAGGACATCCACCTGGGCGTGAACGAGAGCCTGACCGACACCGCCAGAGTG
CTGAGCAGCATGGCCGACGCCGTGCTGGCCCGGGTGTACAAGCAGAGCGACCTGGA
CACCCTGGCCAAAGAGGCCAGCATCCCCATCATCAACGGCCTGAGCGACCTGTACC
ACCCCATCCAGATCCTGGCCGACTACCTGACCCTGCAGGAACACTACAGCTCCCTG
AAGGGCCTGACCCTGAGCTGGATCGGCGACGGCAACAACATCCTGCACAGCATCAT
GATGAGCGCCGCCAAGTTCGGCATGCATCTGCAGGCCGCCACCCCCAAGGGCTACG
AGCCTGATGCCAGCGTGACCAAGCTGGCCGAGCAGTACGCCAAAGAGAACGGCAC
CAAGCTGCTGCTGACCAACGACCCCCTGGAAGCCGCCCACGGCGGCAACGTGCTGA
TCACCGACACCTGGATCAGCATGGGCCAGGAAGAGGAAAAGAAGAAGCGGCTGCA
GGCCTTCCAGGGCTACCAGGTCACAATGAAGACCGCCAAGGTGGCCGCCAGCGACT
GGACCTTCCTGCACTGCCTGCCCCGGAAGCCCGAAGAGGTGGACGACGAGGTGTTC
TACAGCCCCCGGTCCCTGGTGTTCCCCGAGGCCGAGAACCGGAAGTGGACCATTAT
GGCCGTGATGGTGTCCCTGCTGACCGACTACTCCCCCCAGCTGCAGAAGCCCAAGTT
CTAGATAAGTGAA
>pARM1805 (SEQ ID NO: 181)
ATGCTGGTCTTCAACCTGCGGATCCTGCTGAACAACGCCGCCTTCCGGAACGGCCAC
AACTTCATGGTCCGCAACTTCAGATGCGGCCAGCCCCTGCAGAACAGGGTGCAGCT
GAAGGGCCGGGACCTGCTGACCCTGAAGAACTTCACCGGCGAAGAGATCAGGTAC
ATGCTGTGGCTGAGCGCCGACCTGAAGTTCCGGATCAAGCAGAAGGGCGAGTACCT
GCCCCTGCTGCAAGGCAAGAGCCTGGGCATGATCTTCGAGAAGCGGAGCACCCGGA
CCCGGCTGAGCACCGAGACAGGCTTTGCCCTGCTGGGAGGCCACCCCTGCTTTCTGA
CCACCCAGGACATCCACCTGGGCGTGAACGAGAGCCTGACCGACACCGCCAGAGTG
CTGAGCAGCATGGCCGACGCCGTGCTGGCCCGGGTGTACAAGCAGAGCGACCTGGA
CACCCTGGCCAAAGAGGCCAGCATCCCCATCATCAACGGCCTGAGCGACCTGTACC
ACCCCATCCAGATCCTGGCCGACTACCTGACCCTGCAGGAACACTACAGCTCCCTG
AAGGGCCTGACCCTGAGCTGGATCGGCGACGGCAACAACATCCTGCACAGCATCAT
GATGAGCGCCGCCAAGTTCGGCATGCATCTGCAGGCCGCCACCCCCAAGGGCTACG
AGCCTGATGCCAGCGTGACCAAGCTGGCCGAGCAGTACGCCAAAGAGAACGGCAC
CAAGCTGCTGCTGACCAACGACCCCCTGGAAGCCGCCCACGGCGGCAACGTGCTGA
TCACCGACACCTGGATCAGCATGGGCCAGGAAGAGGAAAAGAAGAAGCGGCTGCA
GGCCTTCCAGGGCTACCAGGTCACAATGAAGACCGCCAAGGTGGCCGCCAGCGACT
GGACCTTCCTGCACTGCCTGCCCCGGAAGCCCGAAGAGGTGGACGACGAGGTGTTC
TACAGCCCCCGGTCCCTGGTGTTCCCCGAGGCCGAGAACCGGAAGTGGACCATTAT
GGCCGTGATGGTGTCCCTGCTGACCGACTACTCCCCCCAGCTGCAGAAGCCCAAGTT
CTAGATAAGTGAA
>pARM1806 (SEQ ID NO: 182)
ATGCTGTTCAACCTGAGGATCCTGCTGAACAACGCAGCTTTCAGGAACGGCCACAA
CTTCATGGTGAGGAACTTCCGGTGCGGCCAGCCCCTGCAGAACAAGGTGCAGCTGA
AGGGCAGGGACCTGCTGACCCTGAAGAACTTCACCGGAGAGGAGATCAAGTACAT
GCTGTGGCTGAGCGCAGACCTGAAGTTCAGGATCAAGCAGAAGGGAGAGTACCTGC
CCCTGCTGCAGGGGAAGTCCCTGGGCATGATCTTCGAGAAGAGGAGTACCAGGACC
AGGCTGAGCACCGAAACCGGCTTCGCCCTGCTGGGAGGACACCCCTGCTTCCTGAC
CACCCAGGACATCCACCTGGGCGTGAACGAGAGTCTGACCGACACCGCCAGGGTGC
TGTCTAGCATGGCCGACGCCGTGCTGGCCAGGGTGTACAAGCAGTCAGACCTGGAC
ACCCTGGCTAAGGAGGCCAGCATCCCCATCATCAACGGCCTGAGCGACCTGTACCA
CCCCATCCAGATCCTGGCTGACTACCTGACCCTGCAGGAGCACTACAGCTCTCTGAA
GGGCCTGACCCTGAGCTGGATCGGCGACGGGAACAACATCCTGCACAGCATCATGA
TGAGCGCCGCCAAGTTCGGCATGCACCTGCAGGCCGCTACCCCCAAGGGTTACGAG
CCCGACGCCAGCGTGACCAAGCTGGCAGAGCAGTACGCCAAGGAGAACGGCACCA
AGCTGCTGCTGACCAACGACCCCCTGGAGGCCGCCCACGGAGGCAACGTGCTGATC
ACCGACACCTGGATCAGCATGGGACAGGAGGAGGAGAAGAAGAAGCGGCTGCAGG
CTTTCCAGGGTTACCAGGTGACCATGAAGACCGCCAAGGTGGCTGCCAGCGACTGG
ACCTTCCTGCACTGCCTGCCCAGGAAGCCCGAGGAGGTGGACGACGAGGTGTTCTA
CTCTCCCAGGAGCCTGGTGTTCCCCGAGGCCGAGAACAGGAAGTGGACCATCATGG
CTGTGATGGTGTCCCTGCTGACCGACTACAGCCCCCAGCTGCAGAAGCCCAAGTTCT
GAATAAGTGAA
>pARM1808 (SEQ ID NO: 183)
ATGCTGTTCAACCTGAGGATCCTGCTGAACAACGCAGCTTTCAGGAACGGCCACAA
CTTCATGGTGAGGAACTTCCGGTGCGGCCAGCCCCTGCAGAACAAGGTGCAGCTGA
AGGGCAGGGACCTGCTGACCCTGAAGAACTTCACCGGAGAGGAGATCAAGTACAT
GCTGTGGCTGAGCGCAGACCTGAAGTTCAGGATCAAGCAGAAGGGAGAGTACCTGC
CCCTGCTGCAGGGGAAGTCCCTGGGCATGATCTTCGAGAAGAGGAGTACCAGGACC
AGGCTGAGCACCGAAACCGGCTTCGCCCTGCTGGGAGGACACCCCTGCTTCCTGAC
GACCCAGGACATCCACCTCGGAGTGAACGAATCCCTCACCGATACCGCCCGGGTGT
TATCAAGCATGGCAGATGCCGTGCTGGCCAGGGTGTACAAACAGTCCGATCTGGAC
ACTCTGGCTAAGGAGGCCAGCATCCCCATCATCAACGGCCTGAGCGACCTGTACCA
CCCCATCCAGATCCTGGCTGACTACCTGACCCTGCAGGAGCACTACAGCTCTCTGAA
GGGCCTGACCCTGAGCTGGATCGGCGACGGGAACAACATCCTGCACTCCATCATGA
TGTCCGCCGCGAAGTTCGGAATGCATCTGCAAGCCGCCACGCCAAAAGGATACGAA
CCGGATGCGCCCGTGACAAAGTTGGCGGAACAGTACGCTAAGGAGAACGGAACCA
AGCTGCTGCTGACCAACGACCCCCTGGAGGCCGCCCACGGAGGCAACGTGCTGATC
ACCGACACCTGGATCAGCATGGGACAGGAGGAGGAGAAGAAGAAGCGGCTGCAGG
CTTTCCAGGGTTACCAGGTGACCATGAAGACCGCCAAGGTGGCTGCCAGCGACTGG
ACCTTCCTGCACTGCCTGCCCAGGAAGCCCGAGGAGGTGGACGACGAGGTGTTCTA
CTCTCCCAGGAGCCTGGTGTTCCCCGAGGCCGAGAACAGGAAGTGGACCATCATGG
CTGTGATGGTGTCCCTGCTGACCGACTACAGCCCCCAGCTGCAGAAGCCCAAGTTCT
GAATAAGTGAA
>pARM1809 (SEQ ID NO: 184)
ATGCTGTTCAACCTGCGCATCCTGCTGAACAACGCCGCCTTCCGCAACGGCCACAAC
TTCATGGTGCGCAACTTCCGCTGCGGCCAGCCCCTGCAGAACAAGGTGCAGCTGAA
GGGCCGCGACCTGCTGACCCTGAAGAACTTCACCGGCGAGGAGATCAAGTACATGC
TGTGGCTGAGCGCCGACCTGAAGTTCCGCATCAAGCAGAAGGGCGAGTACCTGCCC
CTGCTGCAGGGCAAGAGCCTGGGCATGATCTTCGAGAAGCGCAGCACCCGCACCCG
CCTGAGCACCGAGACAGGCTTCGCCCTGCTGGGCGGCCACCCCTGCTTCCTGACCAC
CCAGGACATCCACCTGGGCGTGAACGAGAGCCTGACCGACACCGCCCGCGTGCTGA
GCAGCATGGCCGACGCCGTGCTGGCCCGCGTGTACAAGCAGAGCGACCTGGACACC
CTGGCCAAGGAGGCCAGCATCCCCATCATCAACGGCCTGAGCGACCTGTACCACCC
CATCCAGATCCTGGCCGACTACCTGACCCTGCAGGAGCACTACAGCAGCCTGAAGG
GCCTGACCCTGAGCTGGATCGGCGACGGCAACAACATCCTGCACAGCATCATGATG
AGCGCCGCCAAGTTCGGCATGCACCTGCAGGCCGCCACCCCCAAGGGCTACGAGCC
CGACGCCAGCGTGACCAAGCTGGCCGAGCAGTACGCCAAGGAGAACGGCACCAAG
CTGCTGCTGACCAACGACCCCCTGGAGGCCGCCCACGGCGGCAACGTGCTGATCAC
CGACACCTGGATCAGCATGGGCCAGGAGGAGGAGAAGAAGAAGCGCCTGCAGGCC
TTCCAGGGCTACCAGGTGACCATGAAGACCGCCAAGGTGGCCGCCAGCGACTGGAC
CTTCCTGCACTGCCTGCCCCGCAAGCCCGAGGAGGTGGACGACGAGGTGTTCTACA
GCCCCCGCAGCCTGGTGTTCCCCGAGGCCGAGAACCGCAAGTGGACCATCATGGCC
GTGATGGTGAGCCTGCTGACCGACTACAGCCCCCAGCTGCAGAAGCCCAAGTTCTG
AATAAGTGAA
>pARM1816 (SEQ ID NO: 185)
ATGCTGTTCAACCTGCGCATCCTGCTGAACAACGCCGCCTTCCGCAACGGCCACAAC
TTCATGGTGCGCAACTTCCGCTGCGGCCAGCCCCTGCAGAACAAGGTGCAGCTGAA
GGGCCGCGACCTGCTGACCCTGAAGAACTTCACCGGCGAGGAGATCAAGTACATGC
TGTGGCTGAGCGCCGACCTGAAGTTCCGCATCAAGCAGAAGGGCGAGTACCTGCCC
CTGCTGCAGGGCAAGAGCCTGGGCATGATCTTCGAGAAGCGCAGCACCCGCACCCG
CCTGAGCACCGAGACAGGCTTCGCCCTGCTGGGCGGCCACCCCTGCTTCCTGACCAC
CCAGGACATCCACCTGGGCGTGAACGAGAGCCTGACCGACACCGCCCGCGTGCTGA
GCAGCATGGCCGACGCCGTGCTGGCCCGCGTGTACAAGCAGAGCGACCTGGACACC
CTGGCCAAGGAGGCCAGCATCCCCATCATCAACGGCCTGAGCGACCTGTACCACCC
CATCCAGATCCTGGCCGACTACCTGACCCTGCAGGAGCACTACAGCAGCCTGAAGG
GCCTGACCCTGAGCTGGATCGGCGACGGCAACAACATCCTGCACAGCATCATGATG
AGCGCCGCCAAGTTCGGCATGCACCTGCAGGCCGCCACCCCCAAGGGCTACGAGCC
CGACGCCAGCGTGACCAAGCTGGCCGAGCAGTACGCCAAGGAGAACGGCACCAAG
CTGCTGCTGACCAACGACCCCCTGGAGGCCGCCCACGGCGGCAACGTGCTGATCAC
CGACACCTGGATCAGCATGGGCCAGGAGGAGGAGAAGAAGAAGCGCCTGCAGGCC
TTCCAGGGCTACCAGGTGACCATGAAGACCGCCAAGGTGGCCGCCAGCGACTGGAC
CTTCCTGCACTGCCTGCCCCGCAAGCCCGAGGAGGTGGACGACGAGGTGTTCTACA
GCCCCCGCAGCCTGGTGTTCCCCGAGGCCGAGAACCGCAAGTGGACCATCATGGCC
GTGATGGTGAGCCTGCTGACCGACTACAGCCCCCAGCTGCAGAAGCCCAAGTTCTG
AATAAGTGAA
>pARM1822 (SEQ ID NO: 186)
ATGCTTTTCAACTTGAGAATCCTGCTGAACAACGCCGCCTTTCGCAACGGTCACAAT
TTTATGGTCAGAAACTTCAGATGCGGACAGCCCCTCCAAAACAAGGTCCAGCTGAA
GGGCCGCGATCTCCTCACCCTGAAGAACTTCACGGGGGAGGAGATCAAGTACATGC
TGTGGCTCTCCGCTGACCTGAAGTTCAGGATCAAGCAGAAGGGAGAATATCTGCCG
CTGCTGCAAGGGAAGTCCCTGGGGATGATTTTCGAGAAGCGGAGCACCCGGACTCG
GCTCTCCACTGAAACTGGTTTCGCCCTTCTGGGCGGTCACCCCTGCTTCCTGACCAC
TCAAGACATTCACCTCGGAGTGAACGAGTCCTTGACTGACACCGCCCGGGTGCTGT
CGAGCATGGCAGACGCCGTGCTAGCCCGCGTGTACAAGCAGTCAGACCTCGATACC
CTGGCCAAGGAGGCTTCGATCCCGATCATCAACGGGTTGTCCGACCTGTACCACCC
GATTCAGATTCTCGCCGACTACCTCACCCTGCAAGAGCATTACAGCTCCCTGAAGGG
GCTTACCCTGTCCTGGATTGGCGACGGAAACAACATCCTGCACTCCATTATGATGTC
GGCGGCCAAGTTCGGCATGCACCTCCAAGCCGCGACCCCTAAGGGTTACGAACCAG
ACGCGTCAGTGACTAAGCTGGCCGAACAGTACGCAAAGGAAAATGGCACGAAGCT
GCTCCTGACCAACGATCCGTTGGAAGCCGCCCATGGCGGAAATGTGCTCATCACCG
ACACCTGGATCTCGATGGGACAGGAGGAAGAGAAGAAGAAGCGGCTGCAGGCGTT
CCAGGGCTACCAGGTCACCATGAAAACTGCCAAGGTGGCCGCCAGCGACTGGACCT
TCCTGCACTGCCTTCCGCGCAAGCCTGAGGAGGTGGACGATGAAGTGTTCTACTCTC
CACGGTCCCTGGTGTTCCCCGAGGCGGAGAACCGCAAATGGACCATCATGGCTGTG
ATGGTCAGCCTGCTGACCGATTACAGCCCTCAGTTGCAAAAGCCGAAGTTTTGA
>pARM1823 (SEQ ID NO: 187)
ATGCTGTTCAACCTCCGCATCCTCCTCAACAACGCCGCATTCAGAAACGGGCACAA
CTTCATGGTCAGAAACTTCCGCTGCGGGCAACCCCTACAAAACAAGGTCCAGCTCA
AGGGGGGGGACCTCCTGACCCTGAAGAACTTCACCGGCGAAGAGATCAAGTACATG
CTGTGGCTCTCCGCCGACCTGAAGTTCCGCATCAAGCAGAAGGGAGAGTACCTCCC
GCTGCTGCAAGGGAAGTCGCTGGGGATGATCTTCGAGAAGCGGTCAACCAGAACCC
GGCTGTCAACCGAAACCGGGTTCGCACTGCTGGGGGGACACCCGTGCTTCCTGACC
ACCCAAGACATCCACCTGGGAGTGAACGAATCGCTGACCGACACCGCCCGCGTGCT
GAGCTCAATGGCGGACGCCGTGCTGGCCCGCGTGTACAAGCAGTCCGACCTGGACA
CCCTGGCCAAGGAAGCGTCCATCCCGATCATCAACGGACTGTCCGACCTGTACCAC
CCGATCCAGATCCTGGCAGACTACCTGACCCTGCAAGAACACTACAGCTCCCTGAA
GGGCCTGACCCTGTCATGGATCGGGGACGGGAACAACATCCTGCACTCCATAATGA
TGTCAGCCGCCAAGTTCGGAATGCACCTCCAAGCCGCAACCCCGAAGGGCTACGAA
CCGGACGCATCAGTGACCAAACTGGCCGAGCAGTACGCCAAGGAAAACGGCACCA
AGCTCCTGCTGACCAACGACCCGCTGGAGGCCGCACACGGGGGGAACGTGCTGATC
ACCGACACCTGGATCTCCATGGGACAGGAGGAGGAAAAGAAGAAGCGGCTGCAGG
CGTTCCAGGGGTACCAGGTCACCATGAAAACCGCGAAGGTCGCGGCATCAGACTGG
ACCTTCCTGCACTGCCTGCCCCGGAAGCCGGAAGAGGTGGACGACGAGGTGTTCTA
CTCGCCGCGCTCGCTGGTGTTCCCCGAGGCGGAGAACAGGAAGTGGACCATCATGG
CGGTGATGGTCAGCCTCCTGACCGACTACTCGCCGCAGCTGCAGAAGCCGAAGTTC
TGA
>pARM1840 (SEQ ID NO: 188)
ATGCTTTTCAATCTCCGCATCCTCCTTAACAACGCCGCGTTTAGAAACGGCCACAAC
TTCATGGTCCGGAACTTCAGATGTGGCCAGCCGCTTCAAAACAAGGTCCAGCTGAA
GGGCCGGGATCTTCTGACCCTGAAGAACTTTACTGGCGAAGAGATCAAGTACATGC
TCTGGCTCTCCGCGGACTTGAAGTTCCGCATTAAGCAGAAGGGGGAATACCTTCCG
CTGCTTCAAGGAAAGAGCCTCGGCATGATCTTTGAGAAGCGCTCAACCAGGACCCG
CCTTTCTACTGAAACTGGGTTCGCGCTGCTCGGTGGCCACCCCTGCTTCCTGACGAC
CCAGGACATCCACCTCGGAGTGAACGAATCCCTCACCGATACCGCCCGGGTGTTAT
CGAGCATGGCAGATGCCGTGCTGGCCAGGGTGTACAAACAGTCCGATCTGGACACT
CTGGCCAAGGAGGCGTCAATTCCTATTATCAACGGCCTTAGTGACCTCTACCATCCG
ATTCAGATCCTGGCCGATTACCTCACCCTGCAAGAACACTACAGCTCCCTGAAGGGT
CTGACATTGTCCTGGATCGGCGACGGCAACAACATTCTCCATTCCATCATGATGTCC
GCCGCAAAATTCGGCATGCATCTTCAAGCCGCCACGCCGAAGGGTTACGAGCCCGA
CGCTTCCGTGACTAAGCTCGCCGAGCAGTACGCTAAGGAGAACGGAACCAAGCTTC
TGCTGACTAACGACCCACTAGAAGCAGCCCACGGGGGCAACGTGCTTATTACTGAC
ACCTGGATCTCCATGGGCCAGGAAGAAGAGAAAAAGAAGCGGCTGCAGGCGTTCC
AGGGATATCAGGTCACCATGAAAACCGCCAAGGTCGCTGCCTCCGACTGGACCTTC
CTGCACTGCCTGCCTCGCAAGCCTGAAGAAGTGGACGACGAGGTGTTCTACTCGCC
ACGGAGCCTCGTGTTCCCCGAGGCCGAGAATAGAAAGTGGACCATCATGGCCGTGA
TGGTGTCACTGCTCACCGACTACAGCCCGCAGCTTCAGAAGCCCAAGTTCTGAATA
AGTAGA
>pARM1841 (SEQ ID NO: 189)
ATGCTTTTCAACCTGAGAATCCTCTTGAACAATGCTGCTTTTCGGAATGGCCACAAC
TTTATGGTTCGGAACTTCCGTTGCGGCCAGCCTTTACAAAACAAGGTCCAGCTGAAG
GGCCGGGATTTGCTCACACTAAAGAACTTTACTGGAGAAGAGATCAAGTACATGCT
ATGGCTGTCGGCCGACCTGAAGTTCCGTATCAAGCAGAAGGGAGAATACCTTCCGC
TGCTTCAAGGAAAGAGCCTCGGCATGATCTTTGAGAAGCGCTCAACCAGGACCCGC
CTTTCTACTGAAACTGGGTTCGCGCTGCTCGGTGGCCACCCCTGCTTCCTGACGACC
CAGGACATCCACCTCGGAGTGAACGAATCCCTCACCGATACCGCCCGGGTGTTATC
GAGCATGGCAGATGCCGTGCTGGCCAGGGTGTACAAACAGTCCGATCTCGATACCT
TGGCAAAGGAGGCTTCCATTCCCATCATCAACGGCCTGAGCGACCTGTACCACCCA
ATCCAAATCCTGGCTGACTACCTGACCCTGCAAGAGCACTACAGCAGCCTGAAGGG
TCTGACCCTGTCATGGATTGGCGATGGAAACAATATTCTGCACTCCATCATGATGTC
CGCCGCGAAGTTCGGAATGCATCTGCAAGCCGCCACTCCAAAAGGATACGAACCGG
ATGCATCCGTGACCAAGTTGGCGGAACAGTACGCGAAGGAGAACGGAACCAAGCT
CCTGCTGACTAACGACCCGCTCGAGGCTGCGCATGGGGGTAACGTGCTGATTACGG
ACACCTGGATCTCCATGGGGCAGGAGGAAGAGAAGAAGAAGAGACTGCAGGCATT
CCAGGGGTACCAGGTCACCATGAAAACCGCAAAAGTGGCAGCTTCGGACTGGACTT
TCCTGCATTGCCTGCCGAGGAAGCCGGAGGAAGTCGACGACGAAGTGTTCTACTCG
CCTCGGTCCCTGGTGTTCCCCGAGGCCGAAAACCGGAAGTGGACCATCATGGCCGT
GATGGTGTCCTTGCTGACTGACTATAGCCCGCAGCTGCAGAAGCCTAAGTTCTGAAT
AAGTAGA
>pARM1842 (SEQ ID NO: 190)
ATGCTGTTCAACCTGCGCATCCTGCTGAACAACGCCGCCTTCCGCAACGGCCACAAC
TTCATGGTGCGCAACTTCCGCTGCGGCCAGCCCCTGCAGAACAAGGTGCAGCTGAA
GGGCCGCGACCTGCTGACCCTGAAGAACTTCACCGGCGAGGAGATCAAGTACATGC
TGTGGCTGAGCGCCGACCTGAAGTTCCGCATCAAGCAGAAGGGCGAGTACCTGCCC
CTGCTGCAGGGCAAGAGCCTGGGCATGATCTTCGAGAAGCGCAGCACCCGCACCCG
CCTGAGCACCGAGACAGGCCTGGCCCTGCTGGGCGGCCACCCCTGCTTCCTGACCA
CCCAGGACATCCACCTGGGCGTGAACGAGAGCCTGACCGACACCGCCCGCGTGCTG
AGCAGCATGGCCGACGCCGTGCTGGCCCGCGTGTACAAGCAGAGCGACCTGGACAC
CCTGGCCAAGGAGGCCAGCATCCCCATCATCAACGGCCTGAGCGACCTGTACCACC
CCATCCAGATCCTGGCCGACTACCTGACCCTGCAGGAGCACTACAGCAGCCTGAAG
GGCCTGACCCTGAGCTGGATCGGCGACGGCAACAACATCCTGCACAGCATCATGAT
GAGCGCCGCCAAGTTCGGCATGCACCTGCAGGCCGCCACCCCCAAGGGCTACGAGC
CCGACGCCAGCGTGACCAAGCTGGCCGAGCAGTACGCCAAGGAGAACGGCACCAA
GCTGCTGCTGACCAACGACCCCCTGGAGGCCGCCCACGGCGGCAACGTGCTGATCA
CCGACACCTGGATCAGCATGGGCCAGGAGGAGGAGAAGAAGAAGCGCCTGCAGGC
CTTCCAGGGCTACCAGGTGACCATGAAGACCGCCAAGGTGGCCGCCAGCGACTGGA
CCTTCCTGCACTGCCTGCCCCGCAAGCCCGAGGAGGTGGACGACGAGGTGTTCTAC
AGCCCCCGCAGCCTGGTGTTCCCCGAGGCCGAGAACCGCAAGTGGACCATCATGGC
CGTGATGGTGAGCCTGCTGACCGACTACAGCCCCCAGCTGCAGAAGCCCAAGTTCT
GAATAAGTAGA
>pARM1843 (SEQ ID NO: 191)
ATGCTTTTCAATCTCCGCATCCTCCTTAACAACGCCGCGTTTAGAAACGGCCACAAC
TTCATGGTCCGGAACTTCAGATGTGGCCAGCCGCTTCAAAACAAGGTCCAGCTGAA
GGGCCGGGATCTTCTGACCCTGAAGAACTTTACTGGCGAAGAGATCAAGTACATGC
TCTGGCTCTCCGCGGACTTGAAGTTCCGCATTAAGCAGAAGGGGGAATACCTTCCG
CTGCTTCAAGGAAAGAGCCTCGGCATGATCTTTGAGAAGCGCTCAACCAGGACCCG
CCTTTCTACTGAAACTGGGTTCGCGCTGCTCGGTGGCCACCCCTGCTTCCTGACGAC
CCAGGACATCCACCTCGGAGTGAACGAATCCCTCACCGATACCGCCCGGGTGTTAT
CGAGCATGGCAGATGCCGTGCTGGCCAGGGTGTACAAACAGTCCGATCTGGACACT
CTGGCCAAGGAGGCGTCAATTCCTATTATCAACGGCCTTAGTGACCTCTACCATCCG
ATTCAGATCCTGGCCGATTACCTCACCCTGCAAGAACACTACAGCTCCCTGAAGGGT
CTGACATTGTCCTGGATCGGCGACGGCAACAACATTCTCCATTCCATCATGATGTCC
GCCGCAAAATTCGGCATGCATCTTCAAGCCGCCACGCCGAAGGGTTACGAGCCCGA
CGCTTCCGTGACTAAGCTCGCCGAGCAGTACGCTAAGGAGAACGGAACCAAGCTTC
TGCTGACTAACGACCCACTAGAAGCAGCCCACGGGGGCAACGTGCTTATTACTGAC
ACCTGGATCTCCATGGGCCAGGAAGAAGAGAAAAAGAAGCGGCTGCAGGCGTTCC
AGGGATATCAGGTCACCATGAAAACCGCCAAGGTCGCTGCCTCCGACTGGACCTTC
CTGCACTGCCTGCCTCGCAAGCCTGAAGAAGTGGACGACGAGGTGTTCTACTCGCC
ACGGAGCCTCGTGTTCCCCGAGGCCGAGAATAGAAAGTGGACCATCATGGCCGTGA
TGGTGTCACTGCTCACCGACTACAGCCCGCAGCTTCAGAAGCCCAAGTTCTAG
>pARM1844 (SEQ ID NO: 192)
ATGCTTTTCAATCTCCGCATCCTCCTTAACAACGCCGCGTTTAGAAACGGCCACAAC
TTCATGGTCCGGAACTTCAGATGTGGCCAGCCGCTTCAAAACAAGGTCCAGCTGAA
GGGCCGGGATCTTCTGACCCTGAAGAACTTTACTGGCGAAGAGATCAAGTACATGC
TCTGGCTCTCCGCGGACTTGAAGTTCCGCATTAAGCAGAAGGGGGAATACCTTCCG
CTGCTTCAAGGAAAGAGCCTCGGCATGATCTTTGAGAAGCGCTCAACCAGGACCCG
CCTTTCTACTGAAACTGGGTTCGCGCTGCTCGGTGGCCACCCCTGCTTCCTGACGAC
CCAGGACATCCACCTCGGAGTGAACGAATCCCTCACCGATACCGCCCGGGTGTTAT
CGAGCATGGCAGATGCCGTGCTGGCCAGGGTGTACAAACAGTCCGATCTGGACACT
CTGGCCAAGGAGGCGTCAATTCCTATTATCAACGGCCTTAGTGACCTCTACCATCCG
ATTCAGATCCTGGCCGATTACCTCACCCTGCAAGAACACTACAGCTCCCTGAAGGGT
CTGACATTGTCCTGGATCGGCGACGGCAACAACATTCTCCATTCCATCATGATGTCC
GCCGCAAAATTCGGCATGCATCTTCAAGCCGCCACGCCGAAGGGTTACGAGCCCGA
CGCTTCCGTGACTAAGCTCGCCGAGCAGTACGCTAAGGAGAACGGAACCAAGCTTC
TGCTGACTAACGACCCACTAGAAGCAGCCCACGGGGGCAACGTGCTTATTACTGAC
ACCTGGATCTCCATGGGCCAGGAAGAAGAGAAAAAGAAGCGGCTGCAGGCGTTCC
AGGGATATCAGGTCACCATGAAAACCGCCAAGGTCGCTGCCTCCGACTGGACCTTC
CTGCACTGCCTGCCTCGCAAGCCTGAAGAAGTGGACGACGAGGTGTTCTACTCGCC
ACGGAGCCTCGTGTTCCCCGAGGCCGAGAATAGAAAGTGGACCATCATGGCCGTGA
TGGTGTCACTGCTCACCGACTACAGCCCGCAGCTTCAGAAGCCCAAGTTCTAG
>pARM1845 (SEQ ID NO: 193)
ATGCTTTTCAATCTCCGCATCCTCCTTAACAACGCCGCGTTTAGAAACGGCCACAAC
TTCATGGTCCGGAACTTCAGATGTGGCCAGCCGCTTCAAAACAAGGTCCAGCTGAA
GGGCCGGGATCTTCTGACCCTGAAGAACTTTACTGGCGAAGAGATCAAGTACATGC
TCTGGCTCTCCGCGGACTTGAAGTTCCGCATTAAGCAGAAGGGGGAATACCTTCCG
CTGCTTCAAGGAAAGAGCCTCGGCATGATCTTTGAGAAGCGCTCAACCAGGACCCG
CCTTTCTACTGAAACTGGGTTCGCGCTGCTCGGTGGCCACCCCTGCTTCCTGACGAC
CCAGGACATCCACCTCGGAGTGAACGAATCCCTCACCGATACCGCCCGGGTGTTAT
CGAGCATGGCAGATGCCGTGCTGGCCAGGGTGTACAAACAGTCCGATCTGGACACT
CTGGCCAAGGAGGCGTCAATTCCTATTATCAACGGCCTTAGTGACCTCTACCATCCG
ATTCAGATCCTGGCCGATTACCTCACCCTGCAAGAACACTACAGCTCCCTGAAGGGT
CTGACATTGTCCTGGATCGGCGACGGCAACAACATTCTCCATTCCATCATGATGTCC
GCCGCAAAATTCGGCATGCATCTTCAAGCCGCCACGCCGAAGGGTTACGAGCCCGA
CGCTTCCGTGACTAAGCTCGCCGAGCAGTACGCTAAGGAGAACGGAACCAAGCTTC
TGCTGACTAACGACCCACTAGAAGCAGCCCACGGGGGCAACGTGCTTATTACTGAC
ACCTGGATCTCCATGGGCCAGGAAGAAGAGAAAAAGAAGCGGCTGCAGGCGTTCC
AGGGATATCAGGTCACCATGAAAACCGCCAAGGTCGCTGCCTCCGACTGGACCTTC
CTGCACTGCCTGCCTCGCAAGCCTGAAGAAGTGGACGACGAGGTGTTCTACTCGCC
ACGGAGCCTCGTGTTCCCCGAGGCCGAGAATAGAAAGTGGACCATCATGGCCGTGA
TGGTGTCACTGCTCACCGACTACAGCCCGCAGCTTCAGAAGCCCAAGTTCTAG
>pARM1846 (SEQ ID NO: 194)
ATGCTTTTCAATCTCCGCATCCTCCTTAACAACGCCGCGTTTAGAAACGGCCACAAC
TTCATGGTCCGGAACTTCAGATGTGGCCAGCCGCTTCAAAACAAGGTCCAGCTGAA
GGGCCGGGATCTTCTGACCCTGAAGAACTTTACTGGCGAAGAGATCAAGTACATGC
TCTGGCTCTCCGCGGACTTGAAGTTCCGCATTAAGCAGAAGGGGGAATACCTTCCG
CTGCTTCAAGGAAAGAGCCTCGGCATGATCTTTGAGAAGCGCTCAACCAGGACCCG
CCTTTCTACTGAAACTGGGTTCGCGCTGCTCGGTGGCCACCCCTGCTTCCTGACGAC
CCAGGACATCCACCTCGGAGTGAACGAATCCCTCACCGATACCGCCCGGGTGTTAT
CGAGCATGGCAGATGCCGTGCTGGCCAGGGTGTACAAACAGTCCGATCTGGACACT
CTGGCCAAGGAGGCGTCAATTCCTATTATCAACGGCCTTAGTGACCTCTACCATCCG
ATTCAGATCCTGGCCGATTACCTCACCCTGCAAGAACACTACAGCTCCCTGAAGGGT
CTGACATTGTCCTGGATCGGCGACGGCAACAACATTCTCCATTCCATCATGATGTCC
GCCGCAAAATTCGGCATGCATCTTCAAGCCGCCACGCCGAAGGGTTACGAGCCCGA
CGCTTCCGTGACTAAGCTCGCCGAGCAGTACGCTAAGGAGAACGGAACCAAGCTTC
TGCTGACTAACGACCCACTAGAAGCAGCCCACGGGGGCAACGTGCTTATTACTGAC
ACCTGGATCTCCATGGGCCAGGAAGAAGAGAAAAAGAAGCGGCTGCAGGCGTTCC
AGGGATATCAGGTCACCATGAAAACCGCCAAGGTCGCTGCCTCCGACTGGACCTTC
CTGCACTGCCTGCCTCGCAAGCCTGAAGAAGTGGACGACGAGGTGTTCTACTCGCC
ACGGAGCCTCGTGTTCCCCGAGGCCGAGAATAGAAAGTGGACCATCATGGCCGTGA
TGGTGTCACTGCTCACCGACTACAGCCCGCAGCTTCAGAAGCCCAAGTTCTAG
>pARM1847 (SEQ ID NO: 195)
ATGCTTTTCAATCTCCGCATCCTCCTTAACAACGCCGCGTTTAGAAACGGCCACAAC
TTCATGGTCCGGAACTTCAGATGTGGCCAGCCGCTTCAAAACAAGGTCCAGCTGAA
GGGCCGGGATCTTCTGACCCTGAAGAACTTTACTGGCGAAGAGATCAAGTACATGC
TCTGGCTCTCCGCGGACTTGAAGTTCCGCATTAAGCAGAAGGGGGAATACCTTCCG
CTGCTTCAAGGAAAGAGCCTCGGCATGATCTTTGAGAAGCGCTCAACCAGGACCCG
CCTTTCTACTGAAACTGGGTTCGCGCTGCTCGGTGGCCACCCCTGCTTCCTGACGAC
CCAGGACATCCACCTCGGAGTGAACGAATCCCTCACCGATACCGCCCGGGTGTTAT
CGAGCATGGCAGATGCCGTGCTGGCCAGGGTGTACAAACAGTCCGATCTGGACACT
CTGGCCAAGGAGGCGTCAATTCCTATTATCAACGGCCTTAGTGACCTCTACCATCCG
ATTCAGATCCTGGCCGATTACCTCACCCTGCAAGAACACTACAGCTCCCTGAAGGGT
CTGACATTGTCCTGGATCGGCGACGGCAACAACATTCTCCATTCCATCATGATGTCC
GCCGCAAAATTCGGCATGCATCTTCAAGCCGCCACGCCGAAGGGTTACGAGCCCGA
CGCTTCCGTGACTAAGCTCGCCGAGCAGTACGCTAAGGAGAACGGAACCAAGCTTC
TGCTGACTAACGACCCACTAGAAGCAGCCCACGGGGGCAACGTGCTTATTACTGAC
ACCTGGATCTCCATGGGCCAGGAAGAAGAGAAAAAGAAGCGGCTGCAGGCGTTCC
AGGGATATCAGGTCACCATGAAAACCGCCAAGGTCGCTGCCTCCGACTGGACCTTC
CTGCACTGCCTGCCTCGCAAGCCTGAAGAAGTGGACGACGAGGTGTTCTACTCGCC
ACGGAGCCTCGTGTTCCCCGAGGCCGAGAATAGAAAGTGGACCATCATGGCCGTGA
TGGTGTCACTGCTCACCGACTACAGCCCGCAGCTTCAGAAGCCCAAGTTCTAG
>pARM1882 (SEQ ID NO: 196)
ATGGTGTTCAACCTCCGCATCCTCCTCAACAACGCCGCATTCAGAAACGGGCACAA
CTTCATGGTCAGAAACTTCCGCTGCGGGCAACCCCTACAAAACAAGGTCCAGCTCA
AGGGGGGGGACCTCCTGACCCTGAAGAACTTCACCGGCGAAGAGATCAAGTACATG
CTGTGGCTCTCCGCCGACCTGAAGTTCCGCATCAAGCAGAAGGGAGAGTACCTCCC
GCTGCTGCAAGGGAAGTCGCTGGGGATGATCTTCGAGAAGCGGTCAACCAGAACCC
GGCTGTCAACCGAAACCGGGTTCGCACTGCTGGGGGGACACCCGTGCTTCCTGACC
ACCCAAGACATCCACCTGGGAGTGAACGAATCGCTGACCGACACCGCCCGCGTGCT
GAGCTCAATGGCGGACGCCGTGCTGGCCCGCGTGTACAAGCAGTCCGACCTGGACA
CCCTGGCCAAGGAAGCGTCCATCCCGATCATCAACGGACTGTCCGACCTGTACCAC
CCGATCCAGATCCTGGCAGACTACCTGACCCTGCAAGAACACTACAGCTCCCTGAA
GGGCCTGACCCTGTCATGGATCGGGGACGGGAACAACATCCTGCACTCCATAATGA
TGTCAGCCGCCAAGTTCGGAATGCACCTCCAAGCCGCAACCCCGAAGGGCTACGAA
CCGGACGCATCAGTGACCAAACTGGCCGAGCAGTACGCCAAGGAAAACGGCACCA
AGCTCCTGCTGACCAACGACCCGCTGGAGGCCGCACACGGGGGGAACGTGCTGATC
ACCGACACCTGGATCTCCATGGGACAGGAGGAGGAAAAGAAGAAGCGGCTGCAGG
CGTTCCAGGGGTACCAGGTCACCATGAAAACCGCGAAGGTCGCGGCATCAGACTGG
ACCTTCCTGCACTGCCTGCCCCGGAAGCCGGAAGAGGTGGACGACGAGGTGTTCTA
CTCGCCGCGCTCGCTGGTGTTCCCCGAGGCGGAGAACAGGAAGTGGACCATCATGG
CGGTGATGGTCAGCCTCCTGACCGACTACTCGCCGCAGCTGCAGAAGCCGAAGTTC
TGA
>pARM1883 (SEQ ID NO: 197)
ATGGTGTTCAACCTCCGCATCCTCCTCAACAACGCCGCATTCAGAAACGGGCACAA
CTTCATGGTCAGAAACTTCCGCTGCGGGCAACCCCTACAAAACCGGGTCCAGCTCA
AGGGGCGGGACCTCCTGACCCTGAAGAACTTCACCGGCGAAGAGATCAAGTACATG
CTGTGGCTCTCCGCCGACCTGAAGTTCCGCATCAAGCAGAAGGGAGAGTACCTCCC
GCTGCTGCAAGGGAAGTCGCTGGGGATGATCTTCGAGAAGCGGTCAACCAGAACCC
GGCTGTCAACCGAAACCGGGTTCGCACTGCTGGGGGGACACCCGTGCTTCCTGACC
ACCCAAGACATCCACCTGGGAGTGAACGAATCGCTGACCGACACCGCCCGCGTGCT
GAGCTCAATGGCGGACGCCGTGCTGGCCCGCGTGTACAAGCAGTCCGACCTGGACA
CCCTGGCCAAGGAAGCGTCCATCCCGATCATCAACGGACTGTCCGACCTGTACCAC
CCGATCCAGATCCTGGCAGACTACCTGACCCTGCAAGAACACTACAGCTCCCTGAA
GGGCCTGACCCTGTCATGGATCGGGGACGGGAACAACATCCTGCACTCCATAATGA
TGTCAGCCGCCAAGTTCGGAATGCACCTCCAAGCCGCAACCCCGAAGGGCTACGAA
CCGGACGCATCAGTGACCAAACTGGCCGAGCAGTACGCCAAGGAAAACGGCACCA
AGCTCCTGCTGACCAACGACCCGCTGGAGGCCGCACACGGGGGGAACGTGCTGATC
ACCGACACCTGGATCTCCATGGGACAGGAGGAGGAAAAGAAGAAGCGGCTGCAGG
CGTTCCAGGGGTACCAGGTCACCATGAAAACCGCGAAGGTCGCGGCATCAGACTGG
ACCTTCCTGCACTGCCTGCCCCGGAAGCCGGAAGAGGTGGACGACGAGGTGTTCTA
CTCGCCGCGCTCGCTGGTGTTCCCCGAGGCGGAGAACAGGAAGTGGACCATCATGG
CGGTGATGGTCAGCCTCCTGACCGACTACTCGCCGCAGCTGCAGAAGCCGAAGTTC
TGA
>pARM1884 (SEQ ID NO: 198)
ATGGTGTTCAACCTCCGCATCCTCCTCAACAACGCCGCATTCAGAAACGGGCACAA
CTTCATGGTCAGAAACTTCCGCTGCGGGCAACCCCTACAAAACCGGGTCCAGCTCA
AGGGGGGGGACCTCCTGACCCTGAAGAACTTCACCGGCGAAGAGATCCGGTACATG
CTGTGGCTCTCCGCCGACCTGAAGTTCCGCATCAAGCAGAAGGGAGAGTACCTCCC
GCTGCTGCAAGGGAAGTCGCTGGGGATGATCTTCGAGAAGCGGTCAACCAGAACCC
GGCTGTCAACCGAAACCGGGTTCGCACTGCTGGGGGGACACCCGTGCTTCCTGACC
ACCCAAGACATCCACCTGGGAGTGAACGAATCGCTGACCGACACCGCCCGCGTGCT
GAGCTCAATGGCGGACGCCGTGCTGGCCCGCGTGTACAAGCAGTCCGACCTGGACA
CCCTGGCCAAGGAAGCGTCCATCCCGATCATCAACGGACTGTCCGACCTGTACCAC
CCGATCCAGATCCTGGCAGACTACCTGACCCTGCAAGAACACTACAGCTCCCTGAA
GGGCCTGACCCTGTCATGGATCGGGGACGGGAACAACATCCTGCACTCCATAATGA
TGTCAGCCGCCAAGTTCGGAATGCACCTCCAAGCCGCAACCCCGAAGGGCTACGAA
CCGGACGCATCAGTGACCAAACTGGCCGAGCAGTACGCCAAGGAAAACGGCACCA
AGCTCCTGCTGACCAACGACCCGCTGGAGGCCGCACACGGGGGGAACGTGCTGATC
ACCGACACCTGGATCTCCATGGGACAGGAGGAGGAAAAGAAGAAGCGGCTGCAGG
CGTTCCAGGGGTACCAGGTCACCATGAAAACCGCGAAGGTCGCGGCATCAGACTGG
ACCTTCCTGCACTGCCTGCCCCGGAAGCCGGAAGAGGTGGACGACGAGGTGTTCTA
CTCGCCGCGCTCGCTGGTGTTCCCCGAGGCGGAGAACAGGAAGTGGACCATCATGG
CGGTGATGGTCAGCCTCCTGACCGACTACTCGCCGCAGCTGCAGAAGCCGAAGTTC
TGA
>pARM1885 (SEQ ID NO: 199)
ATGCTGGTCAACCTCCGCATCCTCCTCAACAACGCCGCATTCAGAAACGGGCACAA
CTTCATGGTCAGAAACTTCCGCTGCGGGCAACCCCTACAAAACAAGGTCCAGCTCA
AGGGGGGGGACCTCCTGACCCTGAAGAACTTCACCGGCGAAGAGATCAAGTACATG
CTGTGGCTCTCCGCCGACCTGAAGTTCCGCATCAAGCAGAAGGGAGAGTACCTCCC
GCTGCTGCAAGGGAAGTCGCTGGGGATGATCTTCGAGAAGCGGTCAACCAGAACCC
GGCTGTCAACCGAAACCGGGTTCGCACTGCTGGGGGGACACCCGTGCTTCCTGACC
ACCCAAGACATCCACCTGGGAGTGAACGAATCGCTGACCGACACCGCCCGCGTGCT
GAGCTCAATGGCGGACGCCGTGCTGGCCCGCGTGTACAAGCAGTCCGACCTGGACA
CCCTGGCCAAGGAAGCGTCCATCCCGATCATCAACGGACTGTCCGACCTGTACCAC
CCGATCCAGATCCTGGCAGACTACCTGACCCTGCAAGAACACTACAGCTCCCTGAA
GGGCCTGACCCTGTCATGGATCGGGGACGGGAACAACATCCTGCACTCCATAATGA
TGTCAGCCGCCAAGTTCGGAATGCACCTCCAAGCCGCAACCCCGAAGGGCTACGAA
CCGGACGCATCAGTGACCAAACTGGCCGAGCAGTACGCCAAGGAAAACGGCACCA
AGCTCCTGCTGACCAACGACCCGCTGGAGGCCGCACACGGGGGGAACGTGCTGATC
ACCGACACCTGGATCTCCATGGGACAGGAGGAGGAAAAGAAGAAGCGGCTGCAGG
CGTTCCAGGGGTACCAGGTCACCATGAAAACCGCGAAGGTCGCGGCATCAGACTGG
ACCTTCCTGCACTGCCTGCCCCGGAAGCCGGAAGAGGTGGACGACGAGGTGTTCTA
CTCGCCGCGCTCGCTGGTGTTCCCCGAGGCGGAGAACAGGAAGTGGACCATCATGG
CGGTGATGGTCAGCCTCCTGACCGACTACTCGCCGCAGCTGCAGAAGCCGAAGTTC
TGA
>pARM1886 (SEQ ID NO: 200)
ATGCTGGTCAACCTCCGCATCCTCCTCAACAACGCCGCATTCAGAAACGGGCACAA
CTTCATGGTCAGAAACTTCCGCTGCGGGCAACCCCTACAAAACCGGGTCCAGCTCA
AGGGGGGGGACCTCCTGACCCTGAAGAACTTCACCGGCGAAGAGATCAAGTACATG
CTGTGGCTCTCCGCCGACCTGAAGTTCCGCATCAAGCAGAAGGGAGAGTACCTCCC
GCTGCTGCAAGGGAAGTCGCTGGGGATGATCTTCGAGAAGCGGTCAACCAGAACCC
GGCTGTCAACCGAAACCGGGTTCGCACTGCTGGGGGGACACCCGTGCTTCCTGACC
ACCCAAGACATCCACCTGGGAGTGAACGAATCGCTGACCGACACCGCCCGCGTGCT
GAGCTCAATGGCGGACGCCGTGCTGGCCCGCGTGTACAAGCAGTCCGACCTGGACA
CCCTGGCCAAGGAAGCGTCCATCCCGATCATCAACGGACTGTCCGACCTGTACCAC
CCGATCCAGATCCTGGCAGACTACCTGACCCTGCAAGAACACTACAGCTCCCTGAA
GGGCCTGACCCTGTCATGGATCGGGGACGGGAACAACATCCTGCACTCCATAATGA
TGTCAGCCGCCAAGTTCGGAATGCACCTCCAAGCCGCAACCCCGAAGGGCTACGAA
CCGGACGCATCAGTGACCAAACTGGCCGAGCAGTACGCCAAGGAAAACGGCACCA
AGCTCCTGCTGACCAACGACCCGCTGGAGGCCGCACACGGGGGGAACGTGCTGATC
ACCGACACCTGGATCTCCATGGGACAGGAGGAGGAAAAGAAGAAGCGGCTGCAGG
CGTTCCAGGGGTACCAGGTCACCATGAAAACCGCGAAGGTCGCGGCATCAGACTGG
ACCTTCCTGCACTGCCTGCCCCGGAAGCCGGAAGAGGTGGACGACGAGGTGTTCTA
CTCGCCGCGCTCGCTGGTGTTCCCCGAGGCGGAGAACAGGAAGTGGACCATCATGG
CGGTGATGGTCAGCCTCCTGACCGACTACTCGCCGCAGCTGCAGAAGCCGAAGTTC
TGA
>pARM1887 (SEQ ID NO: 201)
ATGCTGGTCAACCTCCGCATCCTCCTCAACAACGCCGCATTCAGAAACGGGCACAA
CTTCATGGTCAGAAACTTCCGCTGCGGGCAACCCCTACAAAACCGGGTCCAGCTCA
AGGGGCGGGACCTCCTGACCCTGAAGAACTTCACCGGCGAAGAGATCCGGTACATG
CTGTGGCTCTCCGCCGACCTGAAGTTCCGCATCAAGCAGAAGGGAGAGTACCTCCC
GCTGCTGCAAGGGAAGTCGCTGGGGATGATCTTCGAGAAGCGGTCAACCAGAACCC
GGCTGTCAACCGAAACCGGGTTCGCACTGCTGGGGGGACACCCGTGCTTCCTGACC
ACCCAAGACATCCACCTGGGAGTGAACGAATCGCTGACCGACACCGCCCGCGTGCT
GAGCTCAATGGCGGACGCCGTGCTGGCCCGCGTGTACAAGCAGTCCGACCTGGACA
CCCTGGCCAAGGAAGCGTCCATCCCGATCATCAACGGACTGTCCGACCTGTACCAC
CCGATCCAGATCCTGGCAGACTACCTGACCCTGCAAGAACACTACAGCTCCCTGAA
GGGCCTGACCCTGTCATGGATCGGGGACGGGAACAACATCCTGCACTCCATAATGA
TGTCAGCCGCCAAGTTCGGAATGCACCTCCAAGCCGCAACCCCGAAGGGCTACGAA
CCGGACGCATCAGTGACCAAACTGGCCGAGCAGTACGCCAAGGAAAACGGCACCA
AGCTCCTGCTGACCAACGACCCGCTGGAGGCCGCACACGGGGGGAACGTGCTGATC
ACCGACACCTGGATCTCCATGGGACAGGAGGAGGAAAAGAAGAAGCGGCTGCAGG
CGTTCCAGGGGTACCAGGTCACCATGAAAACCGCGAAGGTCGCGGCATCAGACTGG
ACCTTCCTGCACTGCCTGCCCCGGAAGCCGGAAGAGGTGGACGACGAGGTGTTCTA
CTCGCCGCGCTCGCTGGTGTTCCCCGAGGCGGAGAACAGGAAGTGGACCATCATGG
CGGTGATGGTCAGCCTCCTGACCGACTACTCGCCGCAGCTGCAGAAGCCGAAGTTC
TGA
>pARM1888 (SEQ ID NO: 202)
ATGCTGGTCAACCTGCGCATCCTGCTGAACAACGCCGCCTTCCGCAACGGCCACAA
CTTCATGGTGCGCAACTTCCGCTGCGGCCAGCCCCTGCAGAACAAGGTGCAGCTGA
AGGGCCGCGACCTGCTGACCCTGAAGAACTTCACCGGCGAGGAGATCAAGTACATG
CTGTGGCTGAGCGCCGACCTGAAGTTCCGCATCAAGCAGAAGGGCGAGTACCTGCC
CCTGCTGCAGGGCAAGAGCCTGGGCATGATCTTCGAGAAGCGCAGCACCCGCACCC
GCCTGAGCACCGAGACAGGCTTCGCCCTGCTGGGCGGCCACCCCTGCTTCCTGACC
ACCCAGGACATCCACCTGGGCGTGAACGAGAGCCTGACCGACACCGCCCGCGTGCT
GAGCAGCATGGCCGACGCCGTGCTGGCCCGCGTGTACAAGCAGAGCGACCTGGACA
CCCTGGCCAAGGAGGCCAGCATCCCCATCATCAACGGCCTGAGCGACCTGTACCAC
CCCATCCAGATCCTGGCCGACTACCTGACCCTGCAGGAGCACTACAGCAGCCTGAA
GGGCCTGACCCTGAGCTGGATCGGCGACGGCAACAACATCCTGCACAGCATCATGA
TGAGCGCCGCCAAGTTCGGCATGCACCTGCAGGCCGCCACCCCCAAGGGCTACGAG
CCCGACGCCAGCGTGACCAAGCTGGCCGAGCAGTACGCCAAGGAGAACGGCACCA
AGCTGCTGCTGACCAACGACCCCCTGGAGGCCGCCCACGGCGGCAACGTGCTGATC
ACCGACACCTGGATCAGCATGGGCCAGGAGGAGGAGAAGAAGAAGCGCCTGCAGG
CCTTCCAGGGCTACCAGGTGACCATGAAGACCGCCAAGGTGGCCGCCAGCGACTGG
ACCTTCCTGCACTGCCTGCCCCGCAAGCCCGAGGAGGTGGACGACGAGGTGTTCTA
CAGCCCCCGCAGCCTGGTGTTCCCCGAGGCCGAGAACCGCAAGTGGACCATCATGG
CCGTGATGGTGAGCCTGCTGACCGACTACAGCCCCCAGCTGCAGAAGCCCAAGTTC
TGA
>pARM1889 (SEQ ID NO: 203)
ATGCTGGTCAACCTGCGCATCCTGCTGAACAACGCCGCCTTCCGCAACGGCCACAA
CTTCATGGTGCGCAACTTCCGCTGCGGCCAGCCCCTGCAGAACCGGGTGCAGCTGA
AGGGCCGCGACCTGCTGACCCTGAAGAACTTCACCGGCGAGGAGATCAAGTACATG
CTGTGGCTGAGCGCCGACCTGAAGTTCCGCATCAAGCAGAAGGGCGAGTACCTGCC
CCTGCTGCAGGGCAAGAGCCTGGGCATGATCTTCGAGAAGCGCAGCACCCGCACCC
GCCTGAGCACCGAGACAGGCTTCGCCCTGCTGGGCGGCCACCCCTGCTTCCTGACC
ACCCAGGACATCCACCTGGGCGTGAACGAGAGCCTGACCGACACCGCCCGCGTGCT
GAGCAGCATGGCCGACGCCGTGCTGGCCCGCGTGTACAAGCAGAGCGACCTGGACA
CCCTGGCCAAGGAGGCCAGCATCCCCATCATCAACGGCCTGAGCGACCTGTACCAC
CCCATCCAGATCCTGGCCGACTACCTGACCCTGCAGGAGCACTACAGCAGCCTGAA
GGGCCTGACCCTGAGCTGGATCGGCGACGGCAACAACATCCTGCACAGCATCATGA
TGAGCGCCGCCAAGTTCGGCATGCACCTGCAGGCCGCCACCCCCAAGGGCTACGAG
CCCGACGCCAGCGTGACCAAGCTGGCCGAGCAGTACGCCAAGGAGAACGGCACCA
AGCTGCTGCTGACCAACGACCCCCTGGAGGCCGCCCACGGCGGCAACGTGCTGATC
ACCGACACCTGGATCAGCATGGGCCAGGAGGAGGAGAAGAAGAAGCGCCTGCAGG
CCTTCCAGGGCTACCAGGTGACCATGAAGACCGCCAAGGTGGCCGCCAGCGACTGG
ACCTTCCTGCACTGCCTGCCCCGCAAGCCCGAGGAGGTGGACGACGAGGTGTTCTA
CAGCCCCCGCAGCCTGGTGTTCCCCGAGGCCGAGAACCGCAAGTGGACCATCATGG
CCGTGATGGTGAGCCTGCTGACCGACTACAGCCCCCAGCTGCAGAAGCCCAAGTTC
TGA
>pARM1890 (SEQ ID NO: 204)
ATGCTTGTCAATCTCCGCATCCTCCTTAACAACGCCGCGTTTAGAAACGGCCACAAC
TTCATGGTCCGGAACTTCAGATGTGGCCAGCCGCTTCAAAACAAGGTCCAGCTGAA
GGGCCGGGATCTTCTGACCCTGAAGAACTTTACTGGCGAAGAGATCAAGTACATGC
TCTGGCTCTCCGCGGACTTGAAGTTCCGCATTAAGCAGAAGGGGGAATACCTTCCG
CTGCTTCAAGGAAAGAGCCTCGGCATGATCTTTGAGAAGCGCTCAACCAGGACCCG
CCTTTCTACTGAAACTGGGTTCGCGCTGCTCGGTGGCCACCCCTGCTTCCTGACGAC
CCAGGACATCCACCTCGGAGTGAACGAATCCCTCACCGATACCGCCCGGGTGTTAT
CGAGCATGGCAGATGCCGTGCTGGCCAGGGTGTACAAACAGTCCGATCTGGACACT
CTGGCCAAGGAGGCGTCAATTCCTATTATCAACGGCCTTAGTGACCTCTACCATCCG
ATTCAGATCCTGGCCGATTACCTCACCCTGCAAGAACACTACAGCTCCCTGAAGGGT
CTGACATTGTCCTGGATCGGCGACGGCAACAACATTCTCCATTCCATCATGATGTCC
GCCGCAAAATTCGGCATGCATCTTCAAGCCGCCACGCCGAAGGGTTACGAGCCCGA
CGCTTCCGTGACTAAGCTCGCCGAGCAGTACGCTAAGGAGAACGGAACCAAGCTTC
TGCTGACTAACGACCCACTAGAAGCAGCCCACGGGGGCAACGTGCTTATTACTGAC
ACCTGGATCTCCATGGGCCAGGAAGAAGAGAAAAAGAAGCGGCTGCAGGCGTTCC
AGGGATATCAGGTCACCATGAAAACCGCCAAGGTCGCTGCCTCCGACTGGACCTTC
CTGCACTGCCTGCCTCGCAAGCCTGAAGAAGTGGACGACGAGGTGTTCTACTCGCC
ACGGAGCCTCGTGTTCCCCGAGGCCGAGAATAGAAAGTGGACCATCATGGCCGTGA
TGGTGTCACTGCTCACCGACTACAGCCCGCAGCTTCAGAAGCCCAAGTTCTAG
>pARM1891 (SEQ ID NO: 205)
ATGCTTGTCAATCTCCGCATCCTCCTTAACAACGCCGCGTTTAGAAACGGCCACAAC
TTCATGGTCCGGAACTTCAGATGTGGCCAGCCGCTTCAAAACCGGGTCCAGCTGAA
GGGCCGGGATCTTCTGACCCTGAAGAACTTTACTGGCGAAGAGATCAAGTACATGC
TCTGGCTCTCCGCGGACTTGAAGTTCCGCATTAAGCAGAAGGGGGAATACCTTCCG
CTGCTTCAAGGAAAGAGCCTCGGCATGATCTTTGAGAAGCGCTCAACCAGGACCCG
CCTTTCTACTGAAACTGGGTTCGCGCTGCTCGGTGGCCACCCCTGCTTCCTGACGAC
CCAGGACATCCACCTCGGAGTGAACGAATCCCTCACCGATACCGCCCGGGTGTTAT
CGAGCATGGCAGATGCCGTGCTGGCCAGGGTGTACAAACAGTCCGATCTGGACACT
CTGGCCAAGGAGGCGTCAATTCCTATTATCAACGGCCTTAGTGACCTCTACCATCCG
ATTCAGATCCTGGCCGATTACCTCACCCTGCAAGAACACTACAGCTCCCTGAAGGGT
CTGACATTGTCCTGGATCGGCGACGGCAACAACATTCTCCATTCCATCATGATGTCC
GCCGCAAAATTCGGCATGCATCTTCAAGCCGCCACGCCGAAGGGTTACGAGCCCGA
CGCTTCCGTGACTAAGCTCGCCGAGCAGTACGCTAAGGAGAACGGAACCAAGCTTC
TGCTGACTAACGACCCACTAGAAGCAGCCCACGGGGGCAACGTGCTTATTACTGAC
ACCTGGATCTCCATGGGCCAGGAAGAAGAGAAAAAGAAGCGGCTGCAGGCGTTCC
AGGGATATCAGGTCACCATGAAAACCGCCAAGGTCGCTGCCTCCGACTGGACCTTC
CTGCACTGCCTGCCTCGCAAGCCTGAAGAAGTGGACGACGAGGTGTTCTACTCGCC
ACGGAGCCTCGTGTTCCCCGAGGCCGAGAATAGAAAGTGGACCATCATGGCCGTGA
TGGTGTCACTGCTCACCGACTACAGCCCGCAGCTTCAGAAGCCCAAGTTCTAG
>pARM1898 (SEQ ID NO: 206)
ATGGGCCTTGTCAATCTCCGCATCCTCCTTAACAACGCCGCGTTTAGAAACGGCCAC
AACTTCATGGTCCGGAACTTCAGATGTGGCCAGCCGCTTCAAAACAAGGTCCAGCT
GAAGGGCCGGGATCTTCTGACCCTGAAGAACTTTACTGGCGAAGAGATCAAGTACA
TGCTCTGGCTCTCCGCGGACTTGAAGTTCCGCATTAAGCAGAAGGGGGAATACCTTC
CGCTGCTTCAAGGAAAGAGCCTCGGCATGATCTTTGAGAAGCGCTCAACCAGGACC
CGCCTTTCTACTGAAACTGGGTTCGCGCTGCTCGGTGGCCACCCCTGCTTCCTGACG
ACCCAGGACATCCACCTCGGAGTGAACGAATCCCTCACCGATACCGCCCGGGTGTT
ATCGAGCATGGCAGATGCCGTGCTGGCCAGGGTGTACAAACAGTCCGATCTGGACA
CTCTGGCCAAGGAGGCGTCAATTCCTATTATCAACGGCCTTAGTGACCTCTACCATC
CGATTCAGATCCTGGCCGATTACCTCACCCTGCAAGAACACTACAGCTCCCTGAAG
GGTCTGACATTGTCCTGGATCGGCGACGGCAACAACATTCTCCATTCCATCATGATG
TCCGCCGCAAAATTCGGCATGCATCTTCAAGCCGCCACGCCGAAGGGTTACGAGCC
CGACGCTTCCGTGACTAAGCTCGCCGAGCAGTACGCTAAGGAGAACGGAACCAAGC
TTCTGCTGACTAACGACCCACTAGAAGCAGCCCACGGGGGCAACGTGCTTATTACT
GACACCTGGATCTCCATGGGCCAGGAAGAAGAGAAAAAGAAGCGGCTGCAGGCGT
TCCAGGGATATCAGGTCACCATGAAAACCGCCAAGGTCGCTGCCTCCGACTGGACC
TTCCTGCACTGCCTGCCTCGCAAGCCTGAAGAAGTGGACGACGAGGTGTTCTACTCG
CCACGGAGCCTCGTGTTCCCCGAGGCCGAGAATAGAAAGTGGACCATCATGGCCGT
GATGGTGTCACTGCTCACCGACTACAGCCCGCAGCTTCAGAAGCCCAAGTTCTAG
>pARM1899 (SEQ ID NO: 207)
ATGGGCCTTGTCAATCTCCGCATCCTCCTTAACAACGCCGCGTTTAGAAACGGCCAC
AACTTCATGGTCCGGAACTTCAGATGTGGCCAGCCGCTTCAAAACCGGGTCCAGCT
GAAGGGCCGGGATCTTCTGACCCTGAAGAACTTTACTGGCGAAGAGATCAAGTACA
TGCTCTGGCTCTCCGCGGACTTGAAGTTCCGCATTAAGCAGAAGGGGGAATACCTTC
CGCTGCTTCAAGGAAAGAGCCTCGGCATGATCTTTGAGAAGCGCTCAACCAGGACC
CGCCTTTCTACTGAAACTGGGTTCGCGCTGCTCGGTGGCCACCCCTGCTTCCTGACG
ACCCAGGACATCCACCTCGGAGTGAACGAATCCCTCACCGATACCGCCCGGGTGTT
ATCGAGCATGGCAGATGCCGTGCTGGCCAGGGTGTACAAACAGTCCGATCTGGACA
CTCTGGCCAAGGAGGCGTCAATTCCTATTATCAACGGCCTTAGTGACCTCTACCATC
CGATTCAGATCCTGGCCGATTACCTCACCCTGCAAGAACACTACAGCTCCCTGAAG
GGTCTGACATTGTCCTGGATCGGCGACGGCAACAACATTCTCCATTCCATCATGATG
TCCGCCGCAAAATTCGGCATGCATCTTCAAGCCGCCACGCCGAAGGGTTACGAGCC
CGACGCTTCCGTGACTAAGCTCGCCGAGCAGTACGCTAAGGAGAACGGAACCAAGC
TTCTGCTGACTAACGACCCACTAGAAGCAGCCCACGGGGGCAACGTGCTTATTACT
GACACCTGGATCTCCATGGGCCAGGAAGAAGAGAAAAAGAAGCGGCTGCAGGCGT
TCCAGGGATATCAGGTCACCATGAAAACCGCCAAGGTCGCTGCCTCCGACTGGACC
TTCCTGCACTGCCTGCCTCGCAAGCCTGAAGAAGTGGACGACGAGGTGTTCTACTCG
CCACGGAGCCTCGTGTTCCCCGAGGCCGAGAATAGAAAGTGGACCATCATGGCCGT
GATGGTGTCACTGCTCACCGACTACAGCCCGCAGCTTCAGAAGCCCAAGTTCTAG
>pARM1900 (SEQ ID NO: 208)
ATGGGCGGACTTGTCAATCTCCGCATCCTCCTTAACAACGCCGCGTTTAGAAACGGC
CACAACTTCATGGTCCGGAACTTCAGATGTGGCCAGCCGCTTCAAAACAAGGTCCA
GCTGAAGGGCCGGGATCTTCTGACCCTGAAGAACTTTACTGGCGAAGAGATCAAGT
ACATGCTCTGGCTCTCCGCGGACTTGAAGTTCCGCATTAAGCAGAAGGGGGAATAC
CTTCCGCTGCTTCAAGGAAAGAGCCTCGGCATGATCTTTGAGAAGCGCTCAACCAG
GACCCGCCTTTCTACTGAAACTGGGTTCGCGCTGCTCGGTGGCCACCCCTGCTTCCT
GACGACCCAGGACATCCACCTCGGAGTGAACGAATCCCTCACCGATACCGCCCGGG
TGTTATCGAGCATGGCAGATGCCGTGCTGGCCAGGGTGTACAAACAGTCCGATCTG
GACACTCTGGCCAAGGAGGCGTCAATTCCTATTATCAACGGCCTTAGTGACCTCTAC
CATCCGATTCAGATCCTGGCCGATTACCTCACCCTGCAAGAACACTACAGCTCCCTG
AAGGGTCTGACATTGTCCTGGATCGGCGACGGCAACAACATTCTCCATTCCATCATG
ATGTCCGCCGCAAAATTCGGCATGCATCTTCAAGCCGCCACGCCGAAGGGTTACGA
GCCCGACGCTTCCGTGACTAAGCTCGCCGAGCAGTACGCTAAGGAGAACGGAACCA
AGCTTCTGCTGACTAACGACCCACTAGAAGCAGCCCACGGGGGCAACGTGCTTATT
ACTGACACCTGGATCTCCATGGGCCAGGAAGAAGAGAAAAAGAAGCGGCTGCAGG
CGTTCCAGGGATATCAGGTCACCATGAAAACCGCCAAGGTCGCTGCCTCCGACTGG
ACCTTCCTGCACTGCCTGCCTCGCAAGCCTGAAGAAGTGGACGACGAGGTGTTCTA
CTCGCCACGGAGCCTCGTGTTCCCCGAGGCCGAGAATAGAAAGTGGACCATCATGG
CCGTGATGGTGTCACTGCTCACCGACTACAGCCCGCAGCTTCAGAAGCCCAAGTTCT
AG
>pARM1903 (SEQ ID NO: 209)
ATGGCCCTTTTCAATCTCCGCATCCTCCTTAACAACGCCGCGTTTAGAAACGGCCAC
AACTTCATGGTCCGGAACTTCAGATGTGGCCAGCCGCTTCAAGGCAAGGTCCAGCT
GAAGGGCCGGGATCTTCTGACCCTGAAGAACTTTACTGGCGAAGAGATCAAGTACA
TGCTCTGGCTCTCCGCGGACTTGAAGTTCCGCATTAAGCAGAAGGGGGAATACCTTC
CGCTGCTTCAAGGAAAGAGCCTCGGCATGATCTTTGAGAAGCGCTCAACCAGGACC
CGCCTTTCTACTGAAACTGGGTTCGCGCTGCTCGGTGGCCACCCCTGCTTCCTGACG
ACCCAGGACATCCACCTCGGAGTGAACGAATCCCTCACCGATACCGCCCGGGTGTT
ATCGAGCATGGCAGATGCCGTGCTGGCCAGGGTGTACAAACAGTCCGATCTGGACA
CTCTGGCCAAGGAGGCGTCAATTCCTATTATCAACGGCCTTAGTGACCTCTACCATC
CGATTCAGATCCTGGCCGATTACCTCACCCTGCAAGAACACTACAGCTCCCTGAAG
GGTCTGACATTGTCCTGGATCGGCGACGGCAACAACATTCTCCATTCCATCATGATG
TCCGCCGCAAAATTCGGCATGCATCTTCAAGCCGCCACGCCGAAGGGTTACGAGCC
CGACGCTTCCGTGACTAAGCTCGCCGAGCAGTACGCTAAGGAGAACGGAACCAAGC
TTCTGCTGACTAACGACCCACTAGAAGCAGCCCACGGGGGCAACGTGCTTATTACT
GACACCTGGATCTCCATGGGCCAGGAAGAAGAGAAAAAGAAGCGGCTGCAGGCGT
TCCAGGGATATCAGGTCACCATGAAAACCGCCAAGGTCGCTGCCTCCGACTGGACC
TTCCTGCACTGCCTGCCTCGCAAGCCTGAAGAAGTGGACGACGAGGTGTTCTACTCG
CCACGGAGCCTCGTGTTCCCCGAGGCCGAGAATAGAAAGTGGACCATCATGGCCGT
GATGGTGTCACTGCTCACCGACTACAGCCCGCAGCTTCAGAAGCCCAAGTTCTAGA
TAAGTGAA
>pARM1904 (SEQ ID NO: 210)
ATGGCCCTTTTCAATCTCCGCATCCTCCTTAACAACGCCGCGTTTAGAAACGGCCAC
AACTTCATGGTCCGGAACTTCAGATGTGGCCAGCCGCTTCAAGGCCGGGTCCAGCT
GAAGGGCCGGGATCTTCTGACCCTGAAGAACTTTACTGGCGAAGAGATCAAGTACA
TGCTCTGGCTCTCCGCGGACTTGAAGTTCCGCATTAAGCAGAAGGGGGAATACCTTC
CGCTGCTTCAAGGAAAGAGCCTCGGCATGATCTTTGAGAAGCGCTCAACCAGGACC
CGCCTTTCTACTGAAACTGGGTTCGCGCTGCTCGGTGGCCACCCCTGCTTCCTGACG
ACCCAGGACATCCACCTCGGAGTGAACGAATCCCTCACCGATACCGCCCGGGTGTT
ATCGAGCATGGCAGATGCCGTGCTGGCCAGGGTGTACAAACAGTCCGATCTGGACA
CTCTGGCCAAGGAGGCGTCAATTCCTATTATCAACGGCCTTAGTGACCTCTACCATC
CGATTCAGATCCTGGCCGATTACCTCACCCTGCAAGAACACTACAGCTCCCTGAAG
GGTCTGACATTGTCCTGGATCGGCGACGGCAACAACATTCTCCATTCCATCATGATG
TCCGCCGCAAAATTCGGCATGCATCTTCAAGCCGCCACGCCGAAGGGTTACGAGCC
CGACGCTTCCGTGACTAAGCTCGCCGAGCAGTACGCTAAGGAGAACGGAACCAAGC
TTCTGCTGACTAACGACCCACTAGAAGCAGCCCACGGGGGCAACGTGCTTATTACT
GACACCTGGATCTCCATGGGCCAGGAAGAAGAGAAAAAGAAGCGGCTGCAGGCGT
TCCAGGGATATCAGGTCACCATGAAAACCGCCAAGGTCGCTGCCTCCGACTGGACC
TTCCTGCACTGCCTGCCTCGCAAGCCTGAAGAAGTGGACGACGAGGTGTTCTACTCG
CCACGGAGCCTCGTGTTCCCCGAGGCCGAGAATAGAAAGTGGACCATCATGGCCGT
GATGGTGTCACTGCTCACCGACTACAGCCCGCAGCTTCAGAAGCCCAAGTTCTAGA
TAAGTGAA
>pARM1905 (SEQ ID NO: 211)
ATGGCCCTTTTCAATCTCCGCATCCTCCTTAACAACGCCGCGTTTAGAAACGGCCAC
AACTTCATGGTCCGGAACTTCAGATGTGGCCAGCCGCTTCAAGGCCGGGTCCAGCT
GAAGGGCCGGGATCTTCTGACCCTGAAGAACTTTACTGGCGAAGAGATCAGGTACA
TGCTCTGGCTCTCCGCGGACTTGAAGTTCCGCATTAAGCAGAAGGGGGAATACCTTC
CGCTGCTTCAAGGAAAGAGCCTCGGCATGATCTTTGAGAAGCGCTCAACCAGGACC
CGCCTTTCTACTGAAACTGGGTTCGCGCTGCTCGGTGGCCACCCCTGCTTCCTGACG
ACCCAGGACATCCACCTCGGAGTGAACGAATCCCTCACCGATACCGCCCGGGTGTT
ATCGAGCATGGCAGATGCCGTGCTGGCCAGGGTGTACAAACAGTCCGATCTGGACA
CTCTGGCCAAGGAGGCGTCAATTCCTATTATCAACGGCCTTAGTGACCTCTACCATC
CGATTCAGATCCTGGCCGATTACCTCACCCTGCAAGAACACTACAGCTCCCTGAAG
GGTCTGACATTGTCCTGGATCGGCGACGGCAACAACATTCTCCATTCCATCATGATG
TCCGCCGCAAAATTCGGCATGCATCTTCAAGCCGCCACGCCGAAGGGTTACGAGCC
CGACGCTTCCGTGACTAAGCTCGCCGAGCAGTACGCTAAGGAGAACGGAACCAAGC
TTCTGCTGACTAACGACCCACTAGAAGCAGCCCACGGGGGCAACGTGCTTATTACT
GACACCTGGATCTCCATGGGCCAGGAAGAAGAGAAAAAGAAGCGGCTGCAGGCGT
TCCAGGGATATCAGGTCACCATGAAAACCGCCAAGGTCGCTGCCTCCGACTGGACC
TTCCTGCACTGCCTGCCTCGCAAGCCTGAAGAAGTGGACGACGAGGTGTTCTACTCG
CCACGGAGCCTCGTGTTCCCCGAGGCCGAGAATAGAAAGTGGACCATCATGGCCGT
GATGGTGTCACTGCTCACCGACTACAGCCCGCAGCTTCAGAAGCCCAAGTTCTAGA
TAAGTGAA
>pARM1906 (SEQ ID NO: 212)
ATGGCCCTTGTCAATCTCCGCATCCTCCTTAACAACGCCGCGTTTAGAAACGGCCAC
AACTTCATGGTCCGGAACTTCAGATGTGGCCAGCCGCTTCAAGGCAGGGTCCAGCT
GAAGGGCCGGGATCTTCTGACCCTGAAGAACTTTACTGGCGAAGAGATCAAGTACA
TGCTCTGGCTCTCCGCGGACTTGAAGTTCCGCATTAAGCAGAAGGGGGAATACCTTC
CGCTGCTTCAAGGAAAGAGCCTCGGCATGATCTTTGAGAAGCGCTCAACCAGGACC
CGCCTTTCTACTGAAACTGGGTTCGCGCTGCTCGGTGGCCACCCCTGCTTCCTGACG
ACCCAGGACATCCACCTCGGAGTGAACGAATCCCTCACCGATACCGCCCGGGTGTT
ATCGAGCATGGCAGATGCCGTGCTGGCCAGGGTGTACAAACAGTCCGATCTGGACA
CTCTGGCCAAGGAGGCGTCAATTCCTATTATCAACGGCCTTAGTGACCTCTACCATC
CGATTCAGATCCTGGCCGATTACCTCACCCTGCAAGAACACTACAGCTCCCTGAAG
GGTCTGACATTGTCCTGGATCGGCGACGGCAACAACATTCTCCATTCCATCATGATG
TCCGCCGCAAAATTCGGCATGCATCTTCAAGCCGCCACGCCGAAGGGTTACGAGCC
CGACGCTTCCGTGACTAAGCTCGCCGAGCAGTACGCTAAGGAGAACGGAACCAAGC
TTCTGCTGACTAACGACCCACTAGAAGCAGCCCACGGGGGCAACGTGCTTATTACT
GACACCTGGATCTCCATGGGCCAGGAAGAAGAGAAAAAGAAGCGGCTGCAGGCGT
TCCAGGGATATCAGGTCACCATGAAAACCGCCAAGGTCGCTGCCTCCGACTGGACC
TTCCTGCACTGCCTGCCTCGCAAGCCTGAAGAAGTGGACGACGAGGTGTTCTACTCG
CCACGGAGCCTCGTGTTCCCCGAGGCCGAGAATAGAAAGTGGACCATCATGGCCGT
GATGGTGTCACTGCTCACCGACTACAGCCCGCAGCTTCAGAAGCCCAAGTTCTAAG
TGAATAGA
>pARM1907 (SEQ ID NO: 213)
ATGGCCCTTTTCAATCTCCGCATCCTCCTTAACAACGCCGCGTTTAGAAACGGCCAC
AACTTCATGGTCCGGAACTTCAGATGTGGCCAGCCGCTTCAAGTCAAGGTCCAGCT
GAAGGGCCGGGATCTTCTGACCCTGAAGAACTTTACTGGCGAAGAGATCAAGTACA
TGCTCTGGCTCTCCGCGGACTTGAAGTTCCGCATTAAGCAGAAGGGGGAATACCTTC
CGCTGCTTCAAGGAAAGAGCCTCGGCATGATCTTTGAGAAGCGCTCAACCAGGACC
CGCCTTTCTACTGAAACTGGGTTCGCGCTGCTCGGTGGCCACCCCTGCTTCCTGACG
ACCCAGGACATCCACCTCGGAGTGAACGAATCCCTCACCGATACCGCCCGGGTGTT
ATCGAGCATGGCAGATGCCGTGCTGGCCAGGGTGTACAAACAGTCCGATCTGGACA
CTCTGGCCAAGGAGGCGTCAATTCCTATTATCAACGGCCTTAGTGACCTCTACCATC
CGATTCAGATCCTGGCCGATTACCTCACCCTGCAAGAACACTACAGCTCCCTGAAG
GGTCTGACATTGTCCTGGATCGGCGACGGCAACAACATTCTCCATTCCATCATGATG
TCCGCCGCAAAATTCGGCATGCATCTTCAAGCCGCCACGCCGAAGGGTTACGAGCC
CGACGCTTCCGTGACTAAGCTCGCCGAGCAGTACGCTAAGGAGAACGGAACCAAGC
TTCTGCTGACTAACGACCCACTAGAAGCAGCCCACGGGGGCAACGTGCTTATTACT
GACACCTGGATCTCCATGGGCCAGGAAGAAGAGAAAAAGAAGCGGCTGCAGGCGT
TCCAGGGATATCAGGTCACCATGAAAACCGCCAAGGTCGCTGCCTCCGACTGGACC
TTCCTGCACTGCCTGCCTCGCAAGCCTGAAGAAGTGGACGACGAGGTGTTCTACTCG
CCACGGAGCCTCGTGTTCCCCGAGGCCGAGAATAGAAAGTGGACCATCATGGCCGT
GATGGTGTCACTGCTCACCGACTACAGCCCGCAGCTTCAGAAGCCCAAGTTCTAGA
TAAGTGAA
>pARM1908 (SEQ ID NO: 214)
ATGGCCCTTTTCAATCTCCGCATCCTCCTTAACAACGCCGCGTTTAGAAACGGCCAC
AACTTCATGGTCCGGAACTTCAGATGTGGCCAGCCGCTTCAAGTCAGGGTCCAGCT
GAAGGGCCGGGATCTTCTGACCCTGAAGAACTTTACTGGCGAAGAGATCAAGTACA
TGCTCTGGCTCTCCGCGGACTTGAAGTTCCGCATTAAGCAGAAGGGGGAATACCTTC
CGCTGCTTCAAGGAAAGAGCCTCGGCATGATCTTTGAGAAGCGCTCAACCAGGACC
CGCCTTTCTACTGAAACTGGGTTCGCGCTGCTCGGTGGCCACCCCTGCTTCCTGACG
ACCCAGGACATCCACCTCGGAGTGAACGAATCCCTCACCGATACCGCCCGGGTGTT
ATCGAGCATGGCAGATGCCGTGCTGGCCAGGGTGTACAAACAGTCCGATCTGGACA
CTCTGGCCAAGGAGGCGTCAATTCCTATTATCAACGGCCTTAGTGACCTCTACCATC
CGATTCAGATCCTGGCCGATTACCTCACCCTGCAAGAACACTACAGCTCCCTGAAG
GGTCTGACATTGTCCTGGATCGGCGACGGCAACAACATTCTCCATTCCATCATGATG
TCCGCCGCAAAATTCGGCATGCATCTTCAAGCCGCCACGCCGAAGGGTTACGAGCC
CGACGCTTCCGTGACTAAGCTCGCCGAGCAGTACGCTAAGGAGAACGGAACCAAGC
TTCTGCTGACTAACGACCCACTAGAAGCAGCCCACGGGGGCAACGTGCTTATTACT
GACACCTGGATCTCCATGGGCCAGGAAGAAGAGAAAAAGAAGCGGCTGCAGGCGT
TCCAGGGATATCAGGTCACCATGAAAACCGCCAAGGTCGCTGCCTCCGACTGGACC
TTCCTGCACTGCCTGCCTCGCAAGCCTGAAGAAGTGGACGACGAGGTGTTCTACTCG
CCACGGAGCCTCGTGTTCCCCGAGGCCGAGAATAGAAAGTGGACCATCATGGCCGT
GATGGTGTCACTGCTCACCGACTACAGCCCGCAGCTTCAGAAGCCCAAGTTCTAGA
TAAGTGAA
>pARM1915 (SEQ ID NO: 215)
ATGCTGTTCAACCTGCGCATCCTGCTGAACAACGCCGCCTTCCGCAACGGCCACAAC
TTCATGGTGCGCAACTTCCGCTGCGGCCAGCCCCTGCAGAACAAGGTGCAGCTGAA
GGGCCGCGACCTGCTGACCCTGAAGAACTTCACCGGCGAGGAGATCAAGTACATGC
TGTGGCTGAGCGCCGACCTGAAGTTCCGCATCAAGCAGAAGGGCGAGTACCTGCCC
CTGCTGCAGGGCAAGAGCCTGGGCATGATCTTCGAGAAGCGCAGCACCCGCACCCG
CCTGAGCACCGAGACAGGCTTCGCCCTGCTGGGCGGCCACCCCTGCTTCCTGACCAC
CCAGGACATCCACCTGGGCGTGAACGAGAGCCTGACCGACACCGCCCGCGTGCTGA
GCAGCATGGCCGACGCCGTGCTGGCCCGCGTGTACAAGCAGAGCGACCTGGACACC
CTGGCCAAGGAGGCCAGCATCCCCATCATCAACGGCCTGAGCGACCTGTACCACCC
CATCCAGATCCTGGCCGACTACCTGACCCTGCAGGAGCACTACAGCAGCCTGAAGG
GCCTGACCCTGAGCTGGATCGGCGACGGCAACAACATCCTGCACAGCATCATGATG
AGCGCCGCCAAGTTCGGCATGCACCTGCAGGCCGCCACCCCCAAGGGCTACGAGCC
CGACGCCAGCGTGACCAAGCTGGCCGAGCAGTACGCCAAGGAGAACGGCACCAAG
CTGCTGCTGACCAACGACCCCCTGGAGGCCGCCCACGGCGGCAACGTGCTGATCAC
CGACACCTGGATCAGCATGGGCCAGGAGGAGGAGAAGAAGAAGCGCCTGCAGGCC
TTCCAGGGCTACCAGGTGACCATGAAGACCGCCAAGGTGGCCGCCAGCGACTGGAC
CTTCCTGCACTGCCTGCCCCGCAAGCCCGAGGAGGTGGACGACGAGGTGTTCTACA
GCCCCCGCAGCCTGGTGTTCCCCGAGGCCGAGAACCGCAAGTGGACCATCATGGCC
GTGATGGTGAGCCTGCTGACCGACTACAGCCCCCAGCTGCAGAAGCCCAAGTTCTG
A
>pARM1916 (SEQ ID NO: 216)
ATGGGAGTATTCAACCTGCGCATCCTGCTGAACAACGCCGCCTTCCGCAACGGCCA
CAACTTCATGGTGCGCAACTTCCGCTGCGGCCAGCCCCTGCAGAACAAGGTGCAGC
TGAAGGGCCGCGACCTGCTGACCCTGAAGAACTTCACCGGCGAGGAGATCAAGTAC
ATGCTGTGGCTGAGCGCCGACCTGAAGTTCCGCATCAAGCAGAAGGGCGAGTACCT
GCCCCTGCTGCAGGGCAAGAGCCTGGGCATGATCTTCGAGAAGCGCAGCACCCGCA
CCCGCCTGAGCACCGAGACAGGCTTCGCCCTGCTGGGCGGCCACCCCTGCTTCCTGA
CCACCCAGGACATCCACCTGGGCGTGAACGAGAGCCTGACCGACACCGCCCGCGTG
CTGAGCAGCATGGCCGACGCCGTGCTGGCCCGCGTGTACAAGCAGAGCGACCTGGA
CACCCTGGCCAAGGAGGCCAGCATCCCCATCATCAACGGCCTGAGCGACCTGTACC
ACCCCATCCAGATCCTGGCCGACTACCTGACCCTGCAGGAGCACTACAGCAGCCTG
AAGGGCCTGACCCTGAGCTGGATCGGCGACGGCAACAACATCCTGCACAGCATCAT
GATGAGCGCCGCCAAGTTCGGCATGCACCTGCAGGCCGCCACCCCCAAGGGCTACG
AGCCCGACGCCAGCGTGACCAAGCTGGCCGAGCAGTACGCCAAGGAGAACGGCAC
CAAGCTGCTGCTGACCAACGACCCCCTGGAGGCCGCCCACGGCGGCAACGTGCTGA
TCACCGACACCTGGATCAGCATGGGCCAGGAGGAGGAGAAGAAGAAGCGCCTGCA
GGCCTTCCAGGGCTACCAGGTGACCATGAAGACCGCCAAGGTGGCCGCCAGCGACT
GGACCTTCCTGCACTGCCTGCCCCGCAAGCCCGAGGAGGTGGACGACGAGGTGTTC
TACAGCCCCCGCAGCCTGGTGTTCCCCGAGGCCGAGAACCGCAAGTGGACCATCAT
GGCCGTGATGGTGAGCCTGCTGACCGACTACAGCCCCCAGCTGCAGAAGCCCAAGT
TCTGA
>pARM1917 (SEQ ID NO: 217)
ATGGGAGTATTCAACCTGCGCATCCTGCTGAACAACGCCGCCTTCCGCAACGGCCA
CAACTTCATGGTGCGCAACTTCCGCTGCGGCCAGCCCCTGCAGAACCGGGTGCAGC
TGAAGGGCCGCGACCTGCTGACCCTGAAGAACTTCACCGGCGAGGAGATCCGGTAC
ATGCTGTGGCTGAGCGCCGACCTGAAGTTCCGCATCAAGCAGAAGGGCGAGTACCT
GCCCCTGCTGCAGGGCAAGAGCCTGGGCATGATCTTCGAGAAGCGCAGCACCCGCA
CCCGCCTGAGCACCGAGACAGGCTTCGCCCTGCTGGGCGGCCACCCCTGCTTCCTGA
CCACCCAGGACATCCACCTGGGCGTGAACGAGAGCCTGACCGACACCGCCCGCGTG
CTGAGCAGCATGGCCGACGCCGTGCTGGCCCGCGTGTACAAGCAGAGCGACCTGGA
CACCCTGGCCAAGGAGGCCAGCATCCCCATCATCAACGGCCTGAGCGACCTGTACC
ACCCCATCCAGATCCTGGCCGACTACCTGACCCTGCAGGAGCACTACAGCAGCCTG
AAGGGCCTGACCCTGAGCTGGATCGGCGACGGCAACAACATCCTGCACAGCATCAT
GATGAGCGCCGCCAAGTTCGGCATGCACCTGCAGGCCGCCACCCCCAAGGGCTACG
AGCCCGACGCCAGCGTGACCAAGCTGGCCGAGCAGTACGCCAAGGAGAACGGCAC
CAAGCTGCTGCTGACCAACGACCCCCTGGAGGCCGCCCACGGCGGCAACGTGCTGA
TCACCGACACCTGGATCAGCATGGGCCAGGAGGAGGAGAAGAAGAAGCGCCTGCA
GGCCTTCCAGGGCTACCAGGTGACCATGAAGACCGCCAAGGTGGCCGCCAGCGACT
GGACCTTCCTGCACTGCCTGCCCCGCAAGCCCGAGGAGGTGGACGACGAGGTGTTC
TACAGCCCCCGCAGCCTGGTGTTCCCCGAGGCCGAGAACCGCAAGTGGACCATCAT
GGCCGTGATGGTGAGCCTGCTGACCGACTACAGCCCCCAGCTGCAGAAGCCCAAGT
TCTGA
>pARM1918 (SEQ ID NO: 218)
ATGCTGGTATTCAACCTGCGCATCCTGCTGAACAACGCCGCCTTCCGCAACGGCCAC
AACTTCATGGTGCGCAACTTCCGCTGCGGCCAGCCCCTGCAGAACCGGGTGCAGCT
GAAGGGCCGCGACCTGCTGACCCTGAAGAACTTCACCGGCGAGGAGATCCGGTACA
TGCTGTGGCTGAGCGCCGACCTGAAGTTCCGCATCAAGCAGAAGGGCGAGTACCTG
CCCCTGCTGCAGGGCAAGAGCCTGGGCATGATCTTCGAGAAGCGCAGCACCCGCAC
CCGCCTGAGCACCGAGACAGGCTTCGCCCTGCTGGGCGGCCACCCCTGCTTCCTGAC
CACCCAGGACATCCACCTGGGCGTGAACGAGAGCCTGACCGACACCGCCCGCGTGC
TGAGCAGCATGGCCGACGCCGTGCTGGCCCGCGTGTACAAGCAGAGCGACCTGGAC
ACCCTGGCCAAGGAGGCCAGCATCCCCATCATCAACGGCCTGAGCGACCTGTACCA
CCCCATCCAGATCCTGGCCGACTACCTGACCCTGCAGGAGCACTACAGCAGCCTGA
AGGGCCTGACCCTGAGCTGGATCGGCGACGGCAACAACATCCTGCACAGCATCATG
ATGAGCGCCGCCAAGTTCGGCATGCACCTGCAGGCCGCCACCCCCAAGGGCTACGA
GCCCGACGCCAGCGTGACCAAGCTGGCCGAGCAGTACGCCAAGGAGAACGGCACC
AAGCTGCTGCTGACCAACGACCCCCTGGAGGCCGCCCACGGCGGCAACGTGCTGAT
CACCGACACCTGGATCAGCATGGGCCAGGAGGAGGAGAAGAAGAAGCGCCTGCAG
GCCTTCCAGGGCTACCAGGTGACCATGAAGACCGCCAAGGTGGCCGCCAGCGACTG
GACCTTCCTGCACTGCCTGCCCCGCAAGCCCGAGGAGGTGGACGACGAGGTGTTCT
ACAGCCCCCGCAGCCTGGTGTTCCCCGAGGCCGAGAACCGCAAGTGGACCATCATG
GCCGTGATGGTGAGCCTGCTGACCGACTACAGCCCCCAGCTGCAGAAGCCCAAGTT
CTGA
>pARM1919 (SEQ ID NO: 219)
ATGGGAGTATTCAACCTGCGCATCCTGCTGAACAACGCCGCCTTCCGCAACGGCCA
CAACTTCATGGTGCGCAACTTCCGCTGCGGCCAGCCCCTGCAGAACAAGGTGCAGC
TGAAGGGCCGCGACCTGCTGACCCTGAAGAACTTCACCGGCGAGGAGATCAAGTAC
ATGCTGTGGCTGAGCGCCGACCTGAAGTTCCGCATCAAGCAGAAGGGCGAGTACCT
GCCCCTGCTGCAGGGCAAGAGCCTGGGCATGATCTTCGAGAAGCGCAGCACCCGCA
CCCGCCTGAGCACCGAGACAGGCTTCGCCCTGCTGGGCGGCCACCCCTGCTTCCTGA
CCACCCAGGACATCCACCTGGGCGTGAACGAGAGCCTGACCGACACCGCCCGCGTG
CTGAGCAGCATGGCCGACGCCGTGCTGGCCCGCGTGTACAAGCAGAGCGACCTGGA
CACCCTGGCCAAGGAGGCCAGCATCCCCATCATCAACGGCCTGAGCGACCTGTACC
ACCCCATCCAGATCCTGGCCGACTACCTGACCCTGCAGGAGCACTACAGCAGCCTG
AAGGGCCTGACCCTGAGCTGGATCGGCGACGGCAACAACATCCTGCACAGCATCAT
GATGAGCGCCGCCAAGTTCGGCATGCACCTGCAGGCCGCCACCCCCAAGGGCTACG
AGCCCGACGCCAGCGTGACCAAGCTGGCCGAGCAGTACGCCAAGGAGAACGGCAC
CAAGCTGCTGCTGACCAACGACCCCCTGGAGGCCGCCCACGGCGGCAACGTGCTGA
TCACCGACACCTGGATCAGCATGGGCCAGGAGGAGGAGAAGAAGAAGCGCCTGCA
GGCCTTCCAGGGCTACCAGGTGACCATGAAGACCGCCAAGGTGGCCGCCAGCGACT
GGACCTTCCTGCACTGCCTGCCCCGCAAGCCCGAGGAGGTGGACGACGAGGTGTTC
TACAGCCCCCGCAGCCTGGTGTTCCCCGAGGCCGAGAACCGCAAGTGGACCATCAT
GGCCGTGATGGTGAGCCTGCTGACCGACTACAGCCCCCAGCTGCAGAAGCCCAAGT
TCTGA
>pARM1920 (SEQ ID NO: 220)
ATGGGAGTATTCAACCTGCGCATCCTGCTGAACAACGCCGCCTTCCGCAACGGCCA
CAACTTCATGGTGCGCAACTTCCGCTGCGGCCAGCCCCTGCAGAACCGGGTGCAGC
TGAAGGGCCGCGACCTGCTGACCCTGAAGAACTTCACCGGCGAGGAGATCCGGTAC
ATGCTGTGGCTGAGCGCCGACCTGAAGTTCCGCATCAAGCAGAAGGGCGAGTACCT
GCCCCTGCTGCAGGGCAAGAGCCTGGGCATGATCTTCGAGAAGCGCAGCACCCGCA
CCCGCCTGAGCACCGAGACAGGCTTCGCCCTGCTGGGCGGCCACCCCTGCTTCCTGA
CCACCCAGGACATCCACCTGGGCGTGAACGAGAGCCTGACCGACACCGCCCGCGTG
CTGAGCAGCATGGCCGACGCCGTGCTGGCCCGCGTGTACAAGCAGAGCGACCTGGA
CACCCTGGCCAAGGAGGCCAGCATCCCCATCATCAACGGCCTGAGCGACCTGTACC
ACCCCATCCAGATCCTGGCCGACTACCTGACCCTGCAGGAGCACTACAGCAGCCTG
AAGGGCCTGACCCTGAGCTGGATCGGCGACGGCAACAACATCCTGCACAGCATCAT
GATGAGCGCCGCCAAGTTCGGCATGCACCTGCAGGCCGCCACCCCCAAGGGCTACG
AGCCCGACGCCAGCGTGACCAAGCTGGCCGAGCAGTACGCCAAGGAGAACGGCAC
CAAGCTGCTGCTGACCAACGACCCCCTGGAGGCCGCCCACGGCGGCAACGTGCTGA
TCACCGACACCTGGATCAGCATGGGCCAGGAGGAGGAGAAGAAGAAGCGCCTGCA
GGCCTTCCAGGGCTACCAGGTGACCATGAAGACCGCCAAGGTGGCCGCCAGCGACT
GGACCTTCCTGCACTGCCTGCCCCGCAAGCCCGAGGAGGTGGACGACGAGGTGTTC
TACAGCCCCCGCAGCCTGGTGTTCCCCGAGGCCGAGAACCGCAAGTGGACCATCAT
GGCCGTGATGGTGAGCCTGCTGACCGACTACAGCCCCCAGCTGCAGAAGCCCAAGT
TCTGA
>pARM1921 (SEQ ID NO: 221)
ATGCTGGTATTCAACCTGCGCATCCTGCTGAACAACGCCGCCTTCCGCAACGGCCAC
AACTTCATGGTGCGCAACTTCCGCTGCGGCCAGCCCCTGCAGAACCGGGTGCAGCT
GAAGGGCCGCGACCTGCTGACCCTGAAGAACTTCACCGGCGAGGAGATCCGGTACA
TGCTGTGGCTGAGCGCCGACCTGAAGTTCCGCATCAAGCAGAAGGGCGAGTACCTG
CCCCTGCTGCAGGGCAAGAGCCTGGGCATGATCTTCGAGAAGCGCAGCACCCGCAC
CCGCCTGAGCACCGAGACAGGCTTCGCCCTGCTGGGCGGCCACCCCTGCTTCCTGAC
CACCCAGGACATCCACCTGGGCGTGAACGAGAGCCTGACCGACACCGCCCGCGTGC
TGAGCAGCATGGCCGACGCCGTGCTGGCCCGCGTGTACAAGCAGAGCGACCTGGAC
ACCCTGGCCAAGGAGGCCAGCATCCCCATCATCAACGGCCTGAGCGACCTGTACCA
CCCCATCCAGATCCTGGCCGACTACCTGACCCTGCAGGAGCACTACAGCAGCCTGA
AGGGCCTGACCCTGAGCTGGATCGGCGACGGCAACAACATCCTGCACAGCATCATG
ATGAGCGCCGCCAAGTTCGGCATGCACCTGCAGGCCGCCACCCCCAAGGGCTACGA
GCCCGACGCCAGCGTGACCAAGCTGGCCGAGCAGTACGCCAAGGAGAACGGCACC
AAGCTGCTGCTGACCAACGACCCCCTGGAGGCCGCCCACGGCGGCAACGTGCTGAT
CACCGACACCTGGATCAGCATGGGCCAGGAGGAGGAGAAGAAGAAGCGCCTGCAG
GCCTTCCAGGGCTACCAGGTGACCATGAAGACCGCCAAGGTGGCCGCCAGCGACTG
GACCTTCCTGCACTGCCTGCCCCGCAAGCCCGAGGAGGTGGACGACGAGGTGTTCT
ACAGCCCCCGCAGCCTGGTGTTCCCCGAGGCCGAGAACCGCAAGTGGACCATCATG
GCCGTGATGGTGAGCCTGCTGACCGACTACAGCCCCCAGCTGCAGAAGCCCAAGTT
CTGA
>pARM1925 (SEQ ID NO: 222)
ATGTTGTTCAACTTGAGGATCTTGTTGAACAACGCCGCCTTCAGGAACGGACACAA
CTTCATGGTAAGGAACTTCAGGTGCGGACAGCCCTTGCAGAACAAAGTACAGTTGA
AAGGAAGGGACTTGTTGACATTGAAAAACTTCACAGGAGAAGAAATCAAATACAT
GTTGTGGTTGTCGGCCGACTTGAAATTCAGGATCAAACAGAAAGGAGAATACTTGC
CCTTGTTGCAGGGAAAATCGTTGGGAATGATCTTCGAAAAAAGGTCGACAAGGACA
AGGTTGTCGACAGAAACAGGATTCGCCTTGTTGGGAGGACACCCCTGCTTCTTGAC
AACACAGGACATCCACTTGGGAGTAAACGAATCGTTGACAGACACAGCCAGGGTAT
TGTCGTCGATGGCCGACGCCGTATTGGCCAGGGTATACAAACAGTCGGACTTGGAC
ACATTGGCCAAAGAAGCCTCGATCCCCATCATCAACGGATTGTCGGACTTGTACCA
CCCCATCCAGATCTTGGCCGACTACTTGACATTGCAGGAACACTACTCGTCGTTGAA
AGGATTGACATTGTCGTGGATCGGAGACGGAAACAACATCTTGCACTCGATCATGA
TGTCGGCCGCCAAATTCGGAATGCACTTGCAGGCCGCCACACCCAAAGGATACGAA
CCCGACGCCTCGGTAACAAAATTGGCCGAACAGTACGCCAAAGAAAACGGAACAA
AATTGTTGTTGACAAACGACCCCTTGGAAGCCGCCCACGGAGGAAACGTATTGATC
ACAGACACATGGATCTCGATGGGACAGGAAGAAGAAAAAAAAAAAAGGTTGCAGG
CCTTCCAGGGATACCAGGTAACAATGAAAACAGCCAAAGTAGCCGCCTCGGACTGG
ACATTCTTGCACTGCTTGCCCAGGAAACCCGAAGAAGTAGACGACGAAGTATTCTA
CTCGCCCAGGTCGTTGGTATTCCCCGAAGCCGAAAACAGGAAATGGACAATCATGG
CCGTAATGGTATCGTTGTTGACAGACTACTCGCCCCAGTTGCAGAAACCCAAATTCT
GAATAGTGAA
>pARM1926 (SEQ ID NO: 223)
ATGCTGTTCAACCTGCGCATCCTGCTGAACAACGCCGCCTTCCGCAACGGCCACAAC
TTCATGGTGCGCAACTTCCGCTGCGGCCAGCCCCTGCAGGGCAAGGTGCAGCTGAA
GGGCCGCGACCTGCTGACCCTGAAGAACTTCACCGGCGAGGAGATCAAGTACATGC
TGTGGCTGAGCGCCGACCTGAAGTTCCGCATCAAGCAGAAGGGCGAGTACCTGCCC
CTGCTGCAGGGCAAGAGCCTGGGCATGATCTTCGAGAAGCGCAGCACCCGCACCCG
CCTGAGCACCGAGACAGGCTTCGCCCTGCTGGGCGGCCACCCCTGCTTCCTGACCAC
CCAGGACATCCACCTGGGCGTGAACGAGAGCCTGACCGACACCGCCCGCGTGCTGA
GCAGCATGGCCGACGCCGTGCTGGCCCGCGTGTACAAGCAGAGCGACCTGGACACC
CTGGCCAAGGAGGCCAGCATCCCCATCATCAACGGCCTGAGCGACCTGTACCACCC
CATCCAGATCCTGGCCGACTACCTGACCCTGCAGGAGCACTACAGCAGCCTGAAGG
GCCTGACCCTGAGCTGGATCGGCGACGGCAACAACATCCTGCACAGCATCATGATG
AGCGCCGCCAAGTTCGGCATGCACCTGCAGGCCGCCACCCCCAAGGGCTACGAGCC
CGACGCCAGCGTGACCAAGCTGGCCGAGCAGTACGCCAAGGAGAACGGCACCAAG
CTGCTGCTGACCAACGACCCCCTGGAGGCCGCCCACGGCGGCAACGTGCTGATCAC
CGACACCTGGATCAGCATGGGCCAGGAGGAGGAGAAGAAGAAGCGCCTGCAGGCC
TTCCAGGGCTACCAGGTGACCATGAAGACCGCCAAGGTGGCCGCCAGCGACTGGAC
CTTCCTGCACTGCCTGCCCCGCAAGCCCGAGGAGGTGGACGACGAGGTGTTCTACA
GCCCCCGCAGCCTGGTGTTCCCCGAGGCCGAGAACCGCAAGTGGACCATCATGGCC
GTGATGGTGAGCCTGCTGACCGACTACAGCCCCCAGCTGCAGAAGCCCAAGTTCTG
A
>pARM1927 (SEQ ID NO: 224)
ATGCTGTTCAACCTGCGCATCCTGCTGAACAACGCCGCCTTCCGCAACGGCCACAAC
TTCATGGTGCGCAACTTCCGCTGCGGCCAGCCCCTGCAGGGCCGGGTGCAGCTGAA
GGGCCGCGACCTGCTGACCCTGAAGAACTTCACCGGCGAGGAGATCAAGTACATGC
TGTGGCTGAGCGCCGACCTGAAGTTCCGCATCAAGCAGAAGGGCGAGTACCTGCCC
CTGCTGCAGGGCAAGAGCCTGGGCATGATCTTCGAGAAGCGCAGCACCCGCACCCG
CCTGAGCACCGAGACAGGCTTCGCCCTGCTGGGCGGCCACCCCTGCTTCCTGACCAC
CCAGGACATCCACCTGGGCGTGAACGAGAGCCTGACCGACACCGCCCGCGTGCTGA
GCAGCATGGCCGACGCCGTGCTGGCCCGCGTGTACAAGCAGAGCGACCTGGACACC
CTGGCCAAGGAGGCCAGCATCCCCATCATCAACGGCCTGAGCGACCTGTACCACCC
CATCCAGATCCTGGCCGACTACCTGACCCTGCAGGAGCACTACAGCAGCCTGAAGG
GCCTGACCCTGAGCTGGATCGGCGACGGCAACAACATCCTGCACAGCATCATGATG
AGCGCCGCCAAGTTCGGCATGCACCTGCAGGCCGCCACCCCCAAGGGCTACGAGCC
CGACGCCAGCGTGACCAAGCTGGCCGAGCAGTACGCCAAGGAGAACGGCACCAAG
CTGCTGCTGACCAACGACCCCCTGGAGGCCGCCCACGGCGGCAACGTGCTGATCAC
CGACACCTGGATCAGCATGGGCCAGGAGGAGGAGAAGAAGAAGCGCCTGCAGGCC
TTCCAGGGCTACCAGGTGACCATGAAGACCGCCAAGGTGGCCGCCAGCGACTGGAC
CTTCCTGCACTGCCTGCCCCGCAAGCCCGAGGAGGTGGACGACGAGGTGTTCTACA
GCCCCCGCAGCCTGGTGTTCCCCGAGGCCGAGAACCGCAAGTGGACCATCATGGCC
GTGATGGTGAGCCTGCTGACCGACTACAGCCCCCAGCTGCAGAAGCCCAAGTTCTG
A
>pARM1928 (SEQ ID NO: 225)
ATGCTGTTCAACCTGCGCATCCTGCTGAACAACGCCGCCTTCCGCAACGGCCACAAC
TTCATGGTGCGCAACTTCCGCTGCGGCCAGCCCCTGCAGGGCCGGGTGCAGCTGAA
GGGCCGCGACCTGCTGACCCTGAAGAACTTCACCGGCGAGGAGATCCGGTACATGC
TGTGGCTGAGCGCCGACCTGAAGTTCCGCATCAAGCAGAAGGGCGAGTACCTGCCC
CTGCTGCAGGGCAAGAGCCTGGGCATGATCTTCGAGAAGCGCAGCACCCGCACCCG
CCTGAGCACCGAGACAGGCTTCGCCCTGCTGGGCGGCCACCCCTGCTTCCTGACCAC
CCAGGACATCCACCTGGGCGTGAACGAGAGCCTGACCGACACCGCCCGCGTGCTGA
GCAGCATGGCCGACGCCGTGCTGGCCCGCGTGTACAAGCAGAGCGACCTGGACACC
CTGGCCAAGGAGGCCAGCATCCCCATCATCAACGGCCTGAGCGACCTGTACCACCC
CATCCAGATCCTGGCCGACTACCTGACCCTGCAGGAGCACTACAGCAGCCTGAAGG
GCCTGACCCTGAGCTGGATCGGCGACGGCAACAACATCCTGCACAGCATCATGATG
AGCGCCGCCAAGTTCGGCATGCACCTGCAGGCCGCCACCCCCAAGGGCTACGAGCC
CGACGCCAGCGTGACCAAGCTGGCCGAGCAGTACGCCAAGGAGAACGGCACCAAG
CTGCTGCTGACCAACGACCCCCTGGAGGCCGCCCACGGCGGCAACGTGCTGATCAC
CGACACCTGGATCAGCATGGGCCAGGAGGAGGAGAAGAAGAAGCGCCTGCAGGCC
TTCCAGGGCTACCAGGTGACCATGAAGACCGCCAAGGTGGCCGCCAGCGACTGGAC
CTTCCTGCACTGCCTGCCCCGCAAGCCCGAGGAGGTGGACGACGAGGTGTTCTACA
GCCCCCGCAGCCTGGTGTTCCCCGAGGCCGAGAACCGCAAGTGGACCATCATGGCC
GTGATGGTGAGCCTGCTGACCGACTACAGCCCCCAGCTGCAGAAGCCCAAGTTCTG
A
>pARM1929 (SEQ ID NO: 226)
ATGCTGTTCAACCTGCGCATCCTGCTGAACAACGCCGCCTTCCGCAACGGCCACAAC
TTCATGGTGCGCAACTTCCGCTGCGGCCAGCCCCTGCAGGGCAAGGTGCAGCTGAA
GGGCCGCGACCTGCTGACCCTGAAGAACTTCACCGGCGAGGAGATCAAGTACATGC
TGTGGCTGAGCGCCGACCTGAAGTTCCGCATCAAGCAGAAGGGCGAGTACCTGCCC
CTGCTGCAGGGCAAGAGCCTGGGCATGATCTTCGAGAAGCGCAGCACCCGCACCCG
CCTGAGCACCGAGACAGGCTTCGCCCTGCTGGGCGGCCACCCCTGCTTCCTGACCAC
CCAGGACATCCACCTGGGCGTGAACGAGAGCCTGACCGACACCGCCCGCGTGCTGA
GCAGCATGGCCGACGCCGTGCTGGCCCGCGTGTACAAGCAGAGCGACCTGGACACC
CTGGCCAAGGAGGCCAGCATCCCCATCATCAACGGCCTGAGCGACCTGTACCACCC
CATCCAGATCCTGGCCGACTACCTGACCCTGCAGGAGCACTACAGCAGCCTGAAGG
GCCTGACCCTGAGCTGGATCGGCGACGGCAACAACATCCTGCACAGCATCATGATG
AGCGCCGCCAAGTTCGGCATGCACCTGCAGGCCGCCACCCCCAAGGGCTACGAGCC
CGACGCCAGCGTGACCAAGCTGGCCGAGCAGTACGCCAAGGAGAACGGCACCAAG
CTGCTGCTGACCAACGACCCCCTGGAGGCCGCCCACGGCGGCAACGTGCTGATCAC
CGACACCTGGATCAGCATGGGCCAGGAGGAGGAGAAGAAGAAGCGCCTGCAGGCC
TTCCAGGGCTACCAGGTGACCATGAAGACCGCCAAGGTGGCCGCCAGCGACTGGAC
CTTCCTGCACTGCCTGCCCCGCAAGCCCGAGGAGGTGGACGACGAGGTGTTCTACA
GCCCCCGCAGCCTGGTGTTCCCCGAGGCCGAGAACCGCAAGTGGACCATCATGGCC
GTGATGGTGAGCCTGCTGACCGACTACAGCCCCCAGCTGCAGAAGCCCAAGTTCTG
A
>pARM2016 (SEQ ID NO: 227)
ATGCTGTTCAACCTGCGCATCCTGCTGAACAACGCCGCCTTCCGCAACGGCCACAAC
TTCATGGTGCGCAACTTCCGCTGCGGCCAGCCCCTGCAGAACAAGGTGCAGCTGAA
GGGCCGCGACCTGCTGACCCTGAAGAACTTCACCGGCGAGGAGATCAAGTACATGC
TGTGGCTGAGCGCCGACCTGAAGTTCCGCATCAAGCAGAAGGGCGAGTACCTGCCC
CTGCTGCAGGGCAAGAGCCTGGGCATGATCTTCGAGAAGCGCAGCACCCGCACCCG
CCTGAGCACCGAGACAGGCTTCGCCCTGCTGGGCGGCCACCCCTGCTTCCTGACCAC
CCAGGACATCCACCTGGGCGTGAACGAGAGCCTGACCGACACCGCCCGCGTGCTGA
GCAGCATGGCCGACGCCGTGCTGGCCCGCGTGTACAAGCAGAGCGACCTGGACACC
CTGGCCAAGGAGGCCAGCATCCCCATCATCAACGGCCTGAGCGACCTGTACCACCC
CATCCAGATCCTGGCCGACTACCTGACCCTGCAGGAGCACTACAGCAGCCTGAAGG
GCCTGACCCTGAGCTGGATCGGCGACGGCAACAACATCCTGCACAGCATCATGATG
AGCGCCGCCAAGTTCGGCATGCACCTGCAGGCCGCCACCCCCAAGGGCTACGAGCC
CGACGCCAGCGTGACCAAGCTGGCCGAGCAGTACGCCAAGGAGAACGGCACCAAG
CTGCTGCTGACCAACGACCCCCTGGAGGCCGCCCACGGCGGCAACGTGCTGATCAC
CGACACCTGGATCAGCATGGGCCAGGAGGAGGAGAAGAAGAAGCGCCTGCAGGCC
TTCCAGGGCTACCAGGTGACCATGAAGACCGCCAAGGTGGCCGCCAGCGACTGGAC
CTTCCTGCACTGCCTGCCCCGCAAGCCCGAGGAGGTGGACGACGAGGTGTTCTACA
GCCCCCGCAGCCTGGTGTTCCCCGAGGCCGAGAACCGCAAGTGGACCATCATGGCC
GTGATGGTGAGCCTGCTGACCGACTACAGCCCCCAGCTGCAGAAGCCCAAGTTCTG
A
>pARM2260 (SEQ ID NO: 228)
ATGCTGGTATTCAACCTGCGCATCCTGCTGAACAACGCCGCCTTCCGCAACGGCCAC
AACTTCATGGTGCGCAACTTCCGCTGCGGCCAGCCCCTGCAGAACCGGGTGCAGCT
GAAGGGCCGCGACCTGCTGACCCTGAAGAACTTCACCGGCGAGGAGATCCGGTACA
TGCTGTGGCTGAGCGCCGACCTGAAGTTCCGCATCAAGCAGAAGGGCGAGTACCTG
CCCCTGCTGCAGGGCAAGAGCCTGGGCATGATCTTCGAGAAGCGCAGCACCCGCAC
CCGCCTGAGCACCGAGACAGGCTTCGCCCTGCTGGGCGGCCACCCCTGCTTCCTGAC
CACCCAGGACATCCACCTGGGCGTGAACGAGAGCCTGACCGACACCGCCCGCGTGC
TGAGCAGCATGGCCGACGCCGTGCTGGCCCGCGTGTACAAGCAGAGCGACCTGGAC
ACCCTGGCCAAGGAGGCCAGCATCCCCATCATCAACGGCCTGAGCGACCTGTACCA
CCCCATCCAGATCCTGGCCGACTACCTGACCCTGCAGGAGCACTACAGCAGCCTGA
AGGGCCTGACCCTGAGCTGGATCGGCGACGGCAACAACATCCTGCACAGCATCATG
ATGAGCGCCGCCAAGTTCGGCATGCACCTGCAGGCCGCCACCCCCAAGGGCTACGA
GCCCGACGCCAGCGTGACCAAGCTGGCCGAGCAGTACGCCAAGGAGAACGGCACC
AAGCTGCTGCTGACCAACGACCCCCTGGAGGCCGCCCACGGCGGCAACGTGCTGAT
CACCGACACCTGGATCAGCATGGGCCAGGAGGAGGAGAAGAAGAAGCGCCTGCAG
GCCTTCCAGGGCTACCAGGTGACCATGAAGACCGCCAAGGTGGCCGCCAGCGACTG
GACCTTCCTGCACTGCCTGCCCCGCAAGCCCGAGGAGGTGGACGACGAGGTGTTCT
ACAGCCCCCGCAGCCTGGTGTTCCCCGAGGCCGAGAACCGCAAGTGGACCATCATG
GCCGTGATGGTGAGCCTGCTGACCGACTACAGCCCCCAGCTGCAGAAGCCCAAGTT
CTGA
>pARM2262 (SEQ ID NO: 229)
ATGCTGGTATTCAACCTGCGCATCCTGCTGAACAACGCCGCCTTCCGCAACGGCCAC
AACTTCATGGTGCGCAACTTCCGCTGCGGCCAGCCCCTGCAGAACCGGGTGCAGCT
GAAGGGCCGCGACCTGCTGACCCTGAAGAACTTCACCGGCGAGGAGATCCGGTACA
TGCTGTGGCTGAGCGCCGACCTGAAGTTCCGCATCAAGCAGAAGGGCGAGTACCTG
CCCCTGCTGCAGGGCAAGAGCCTGGGCATGATCTTCGAGAAGCGCAGCACCCGCAC
CCGCCTGAGCACCGAGACAGGCTTCGCCCTGCTGGGCGGCCACCCCTGCTTCCTGAC
CACCCAGGACATCCACCTGGGCGTGAACGAGAGCCTGACCGACACCGCCCGCGTGC
TGAGCAGCATGGCCGACGCCGTGCTGGCCCGCGTGTACAAGCAGAGCGACCTGGAC
ACCCTGGCCAAGGAGGCCAGCATCCCCATCATCAACGGCCTGAGCGACCTGTACCA
CCCCATCCAGATCCTGGCCGACTACCTGACCCTGCAGGAGCACTACAGCAGCCTGA
AGGGCCTGACCCTGAGCTGGATCGGCGACGGCAACAACATCCTGCACAGCATCATG
ATGAGCGCCGCCAAGTTCGGCATGCACCTGCAGGCCGCCACCCCCAAGGGCTACGA
GCCCGACGCCAGCGTGACCAAGCTGGCCGAGCAGTACGCCAAGGAGAACGGCACC
AAGCTGCTGCTGACCAACGACCCCCTGGAGGCCGCCCACGGCGGCAACGTGCTGAT
CACCGACACCTGGATCAGCATGGGCCAGGAGGAGGAGAAGAAGAAGCGCCTGCAG
GCCTTCCAGGGCTACCAGGTGACCATGAAGACCGCCAAGGTGGCCGCCAGCGACTG
GACCTTCCTGCACTGCCTGCCCCGCAAGCCCGAGGAGGTGGACGACGAGGTGTTCT
ACAGCCCCCGCAGCCTGGTGTTCCCCGAGGCCGAGAACCGCAAGTGGACCATCATG
GCCGTGATGGTGAGCCTGCTGACCGACTACAGCCCCCAGCTGCAGAAGCCCAAGTT
CTGA

| | | |
|---|---|---|
| AT5G61250 | AACCAAUCGAAAGAAACCAAA | SEQ ID NO: 230 |
| AT5G46430 | CUCUAAUCACCAGGAGUAAAA | SEQ ID NO: 231 |
| AT5G47110 | GAGAGAGAUCUUAACAAAAAA | SEQ ID NO: 232 |
| AT1G03110 | UGUGUAACAACAACAACAACA | SEQ ID NO: 233 |
| AT3G12380 | CCGCAGUAGGAAGAGAAAGCC | SEQ ID NO: 234 |
| AT5G45910 | AAAAAAAAAAGAAAUCAUAAA | SEQ ID NO: 235 |
| AT1G07260 | GAGAGAAGAAAGAAGAAGACG | SEQ ID NO: 236 |
| AT3G55500 | CAAUUAAAAAUACUUACCAAA | SEQ ID NO: 237 |
| AT3G46230 | GCAAACAGAGUAAGCGAAACG | SEQ ID NO: 238 |
| AT2G36170 | GCGAAGAAGACGAACGCAAAG | SEQ ID NO: 239 |
| AT1G10660 | UUAGGACUGUAUUGACUGGCC | SEQ ID NO: 240 |
| AT4G14340 | AUCAUCGGAAUUCGGAAAAAG | SEQ ID NO: 241 |
| AT1G49310 | AAAACAAAAGUUAAAGCAGAC | SEQ ID NO: 242 |
| AT4G14360 | UUUAUCUCAAAUAAGAAGGCA | SEQ ID NO: 243 |
| AT1G28520 | GGUGGGGAGGUGAGAUUUCUU | SEQ ID NO: 244 |
| AT1G20160 | UGAUUAGGAAACUACAAAGCC | SEQ ID NO: 245 |
| AT5G37370 | CAUUUUUCAAUUUCAUAAAAC | SEQ ID NO: 246 |
| AT4G11320 | UUACUUUUAAGCCCAACAAAA | SEQ ID NO: 247 |
| AT5G40850 | GGCGUGUGUGUGUGUUGUUGA | SEQ ID NO: 248 |
| AT1G06150 | GUGGUGAAGGGGAAGGUUUAG | SEQ ID NO: 249 |
| AT2G26080 | UUGUUUUUUUUUGGUUUGGUU | SEQ ID NO:250 |

mARM2260 (SEQ ID NO: 251)
AGGAUUAUUACAUCAAAACAAAAAGCCGCCACCAUGCUGGUAUUCAACCUGCGC
AUCCUGCUGAACAACGCCGCCUUCCGCAACGGCCACAACUUCAUGGUGCGCAACU
UCCGCUGCGGCCAGCCCCUGCAGAACCGGGUGCAGCUGAAGGGCCGCGACCUGCU
GACCCUGAAGAACUUCACCGGCGAGGAGAUCCGGUACAUGCUGUGGCUGAGCGC
CGACCUGAAGUUCCGCAUCAAGCAGAAGGGCGAGUACCUGCCCCUGCUGCAGGGC
AAGAGCCUGGGCAUGAUCUUCGAGAAGCGCAGCACCCGCACCCGCCUGAGCACCG
AGACAGGCUUCGCCCUGCUGGGCGGCCACCCCUGCUUCCUGACCACCCAGGACAU
CCACCUGGGCGUGAACGAGAGCCUGACCGACACCGCCCGCGUGCUGAGCAGCAUG
GCCGACGCCGUGCUGGCCCGCGUGUACAAGCAGAGCGACCUGGACACCCUGGCCA
AGGAGGCCAGCAUCCCCAUCAUCAACGGCCUGAGCGACCUGUACCACCCCAUCCA
GAUCCUGGCCGACUACCUGACCCUGCAGGAGCACUACAGCAGCCUGAAGGGCCUG
ACCCUGAGCUGGAUCGGCGACGGCAACAACAUCCUGCACAGCAUCAUGAUGAGCG
CCGCCAAGUUCGGCAUGCACCUGCAGGCCGCCACCCCCAAGGGCUACGAGCCCGA
CGCCAGCGUGACCAAGCUGGCCGAGCAGUACGCCAAGGAGAACGGCACCAAGCUG
CUGCUGACCAACGACCCCCUGGAGGCCGCCCACGGCGGCAACGUGCUGAUCACCG
ACACCUGGAUCAGCAUGGGCCAGGAGGAGGAGAAGAAGAAGCGCCUGCAGGCCU
UCCAGGGCUACCAGGUGACCAUGAAGACCGCCAAGGUGGCCGCCAGCGACUGGAC
CUUCCUGCACUGCCUGCCCCGCAAGCCCGAGGAGGUGGACGACGAGGUGUUCUAC
AGCCCCCGCAGCCUGGUGUUCCCCGAGGCCGAGAACCGCAAGUGGACCAUCAUGG
CCGUGAUGGUGAGCCUGCUGACCGACUACAGCCCCCAGCUGCAGAAGCCCAAGUU
CUGAGGUCUCUAGUAAUGAGCUGGAGCCUCGGUAGCCGUUCCUCCUGCCCGCUGG
GCCUCCCAACGGGCCCUCCUCCCCUCCUUGCACCGGCCCUUCCUGGUCUUUGAAU
AAAGUCUGAGUGGGCAGCAUCUAG
mARM2262 (SEQ ID NO: 252)
AGGAUUAUUACAUCAAAACAAAAAGCCGCCACCAUGCUGGUAUUCAACCUGCGC
AUCCUGCUGAACAACGCCGCCUUCCGCAACGGCCACAACUUCAUGGUGCGCAACU
UCCGCUGCGGCCAGCCCCUGCAGAACCGGGUGCAGCUGAAGGGCCGCGACCUGCU
GACCCUGAAGAACUUCACCGGCGAGGAGAUCCGGUACAUGCUGUGGCUGAGCGC
CGACCUGAAGUUCCGCAUCAAGCAGAAGGGCGAGUACCUGCCCCUGCUGCAGGGC
AAGAGCCUGGGCAUGAUCUUCGAGAAGCGCAGCACCCGCACCCGCCUGAGCACCG
AGACAGGCUUCGCCCUGCUGGGCGGCCACCCCUGCUUCCUGACCACCCAGGACAU
CCACCUGGGCGUGAACGAGAGCCUGACCGACACCGCCCGCGUGCUGAGCAGCAUG
GCCGACGCCGUGCUGGCCCGCGUGUACAAGCAGAGCGACCUGGACACCCUGGCCA
AGGAGGCCAGCAUCCCCAUCAUCAACGGCCUGAGCGACCUGUACCACCCCAUCCA
GAUCCUGGCCGACUACCUGACCCUGCAGGAGCACUACAGCAGCCUGAAGGGCCUG
ACCCUGAGCUGGAUCGGCGACGGCAACAACAUCCUGCACAGCAUCAUGAUGAGCG
CCGCCAAGUUCGGCAUGCACCUGCAGGCCGCCACCCCCAAGGGCUACGAGCCCGA
CGCCAGCGUGACCAAGCUGGCCGAGCAGUACGCCAAGGAGAACGGCACCAAGCUG
CUGCUGACCAACGACCCCCUGGAGGCCGCCCACGGCGGCAACGUGCUGAUCACCG
ACACCUGGAUCAGCAUGGGCCAGGAGGAGGAGAAGAAGAAGCGCCUGCAGGCCU
UCCAGGGCUACCAGGUGACCAUGAAGACCGCCAAGGUGGCCGCCAGCGACUGGAC
CUUCCUGCACUGCCUGCCCCGCAAGCCCGAGGAGGUGGACGACGAGGUGUUCUAC
AGCCCCCGCAGCCUGGUGUUCCCCGAGGCCGAGAACCGCAAGUGGACCAUCAUGG
CCGUGAUGGUGAGCCUGCUGACCGACUACAGCCCCCAGCUGCAGAAGCCCAAGUU
CUGAGGUCUCUAGUAAUGAGCUGGAGCCUCGGUAGCCGUUCCUCCUGCCCGCUGG
GCCUCCCAACGGGCCCUCCUCCCCUCCUUGCACCGGCCCUUCCUGGUCUUUGAAU
AAAGUCUGAGUGGGCAGCAUCUAG
mARM2016 (SEQ ID NO: 253)
AGGAUUAUUACAUCAAAACAAAAAGCCGCCACCAUGCUGUUCAACCUGCGCAUCC
UGCUGAACAACGCCGCCUUCCGCAACGGCCACAACUUCAUGGUGCGCAACUUCCG
CUGCGGCCAGCCCCUGCAGAACAAGGUGCAGCUGAAGGGCCGCGACCUGCUGACC
CUGAAGAACUUCACCGGCGAGGAGAUCAAGUACAUGCUGUGGCUGAGCGCCGAC
CUGAAGUUCCGCAUCAAGCAGAAGGGCGAGUACCUGCCCCUGCUGCAGGGCAAG
AGCCUGGGCAUGAUCUUCGAGAAGCGCAGCACCCGCACCCGCCUGAGCACCGAGA
CAGGCUUCGCCCUGCUGGGCGGCCACCCCUGCUUCCUGACCACCCAGGACAUCCA
CCUGGGCGUGAACGAGAGCCUGACCGACACCGCCCGCGUGCUGAGCAGCAUGGCC
GACGCCGUGCUGGCCCGCGUGUACAAGCAGAGCGACCUGGACACCCUGGCCAAGG
AGGCCAGCAUCCCCAUCAUCAACGGCCUGAGCGACCUGUACCACCCCAUCCAGAU
CCUGGCCGACUACCUGACCCUGCAGGAGCACUACAGCAGCCUGAAGGGCCUGACC
CUGAGCUGGAUCGGCGACGGCAACAACAUCCUGCACAGCAUCAUGAUGAGCGCCG
CCAAGUUCGGCAUGCACCUGCAGGCCGCCACCCCCAAGGGCUACGAGCCCGACGC
CAGCGUGACCAAGCUGGCCGAGCAGUACGCCAAGGAGAACGGCACCAAGCUGCUG
CUGACCAACGACCCCCUGGAGGCCGCCCACGGCGGCAACGUGCUGAUCACCGACA
CCUGGAUCAGCAUGGGCCAGGAGGAGGAGAAGAAGAAGCGCCUGCAGGCCUUCC
AGGGCUACCAGGUGACCAUGAAGACCGCCAAGGUGGCCGCCAGCGACUGGACCUU
CCUGCACUGCCUGCCCCGCAAGCCCGAGGAGGUGGACGACGAGGUGUUCUACAGC
CCCCGCAGCCUGGUGUUCCCCGAGGCCGAGAACCGCAAGUGGACCAUCAUGGCCG
UGAUGGUGAGCCUGCUGACCGACUACAGCCCCCAGCUGCAGAAGCCCAAGUUCUG
AGGUCUCUAGUAAUGAGCUGGAGCCUCGGUAGCCGUUCCUCCUGCCCGCUGGGCC
UCCCAACGGGCCCUCCUCCCCUCCUUGCACCGGCCCUUCCUGGUCUUUGAAUAAA
GUCUGAGUGGGCAGCAUCUAG
Open Reading Frame of Construct 1799 (SEQ ID NO: 254)
AUGCUGUUCAACCUGCGCAUCCUGCUGAACAACGCCGCCUUCCGCAACGGCCACA
ACUUCAUGGUGCGCAACUUCCGCUGCGGCCAGCCCCUGCAGAACAAGGUGCAGCU
GAAGGGCCGCGACCUGCUGACCCUGAAGAACUUCACCGGCGAGGAGAUCAAGUA
CAUGCUGUGGCUGAGCGCCGACCUGAAGUUCCGCAUCAAGCAGAAGGGCGAGUA
CCUGCCCCUGCUGCAGGGCAAGAGCCUGGGCAUGAUCUUCGAGAAGCGCAGCACC
CGCACCCGCCUGAGCACCGAGACAGGCUUCGCCCUGCUGGGCGGCCACCCCUGCU
UCCUGACCACCCAGGACAUCCACCUGGGCGUGAACGAGAGCCUGACCGACACCGC
CCGCGUGCUGAGCAGCAUGGCCGACGCCGUGCUGGCCCGCGUGUACAAGCAGAGC
GACCUGGACACCCUGGCCAAGGAGGCCAGCAUCCCCAUCAUCAACGGCCUGAGCG
ACCUGUACCACCCCAUCCAGAUCCUGGCCGACUACCUGACCCUGCAGGAGCACUA
CAGCAGCCUGAAGGGCCUGACCCUGAGCUGGAUCGGCGACGGCAACAACAUCCUG
CACAGCAUCAUGAUGAGCGCCGCCAAGUUCGGCAUGCACCUGCAGGCCGCCACCC
CCAAGGGCUACGAGCCCGACGCCAGCGUGACCAAGCUGGCCGAGCAGUACGCCAA
GGAGAACGGCACCAAGCUGCUGCUGACCAACGACCCCCUGGAGGCCGCCCACGGC
GGCAACGUGCUGAUCACCGACACCUGGAUCAGCAUGGGCCAGGAGGAGGAGAAG
AAGAAGCGCCUGCAGGCCUUCCAGGGCUACCAGGUGACCAUGAAGACCGCCAAGG
UGGCCGCCAGCGACUGGACCUUCCUGCACUGCCUGCCCCGCAAGCCCGAGGAGGU
GGACGACGAGGUGUUCUACAGCCCCCGCAGCCUGGUGUUCCCCGAGGCCGAGAAC
CGCAAGUGGACCAUCAUGGCCGUGAUGGUGAGCCUGCUGACCGACUACAGCCCCC
AGCUGCAGAAGCCCAAGUUCUGA
Open Reading Frame of Construct 1921 (SEQ ID NO: 255)
AUGCUGGUAUUCAACCUGCGCAUCCUGCUGAACAACGCCGCCUUCCGCAACGGCC
ACAACUUCAUGGUGCGCAACUUCCGCUGCGGCCAGCCCCUGCAGAACCGGGUGCA
GCUGAAGGGCCGCGACCUGCUGACCCUGAAGAACUUCACCGGCGAGGAGAUCCGG
UACAUGCUGUGGCUGAGCGCCGACCUGAAGUUCCGCAUCAAGCAGAAGGGCGAG
UACCUGCCCCUGCUGCAGGGCAAGAGCCUGGGCAUGAUCUUCGAGAAGCGCAGCA
CCCGCACCCGCCUGAGCACCGAGACAGGCUUCGCCCUGCUGGGCGGCCACCCCUG
CUUCCUGACCACCCAGGACAUCCACCUGGGCGUGAACGAGAGCCUGACCGACACC
GCCCGCGUGCUGAGCAGCAUGGCCGACGCCGUGCUGGCCCGCGUGUACAAGCAGA
GCGACCUGGACACCCUGGCCAAGGAGGCCAGCAUCCCCAUCAUCAACGGCCUGAG
CGACCUGUACCACCCCAUCCAGAUCCUGGCCGACUACCUGACCCUGCAGGAGCAC
UACAGCAGCCUGAAGGGCCUGACCCUGAGCUGGAUCGGCGACGGCAACAACAUCC
UGCACAGCAUCAUGAUGAGCGCCGCCAAGUUCGGCAUGCACCUGCAGGCCGCCAC
CCCCAAGGGCUACGAGCCCGACGCCAGCGUGACCAAGCUGGCCGAGCAGUACGCC
AAGGAGAACGGCACCAAGCUGCUGCUGACCAACGACCCCCUGGAGGCCGCCCACG
GCGGCAACGUGCUGAUCACCGACACCUGGAUCAGCAUGGGCCAGGAGGAGGAGA
AGAAGAAGCGCCUGCAGGCCUUCCAGGGCUACCAGGUGACCAUGAAGACCGCCAA
GGUGGCCGCCAGCGACUGGACCUUCCUGCACUGCCUGCCCCGCAAGCCCGAGGAG
GUGGACGACGAGGUGUUCUACAGCCCCCGCAGCCUGGUGUUCCCCGAGGCCGAGA
ACCGCAAGUGGACCAUCAUGGCCGUGAUGGUGAGCCUGCUGACCGACUACAGCCC
CCAGCUGCAGAAGCCCAAGUUCUGA
Open Reading Frame of Construct 2016 (SEQ ID NO: 256)
AUGCUGUUCAACCUGCGCAUCCUGCUGAACAACGCCGCCUUCCGCAACGGCCACA
ACUUCAUGGUGCGCAACUUCCGCUGCGGCCAGCCCCUGCAGAACAAGGUGCAGCU
GAAGGGCCGCGACCUGCUGACCCUGAAGAACUUCACCGGCGAGGAGAUCAAGUA
CAUGCUGUGGCUGAGCGCCGACCUGAAGUUCCGCAUCAAGCAGAAGGGCGAGUA
CCUGCCCCUGCUGCAGGGCAAGAGCCUGGGCAUGAUCUUCGAGAAGCGCAGCACC
CGCACCCGCCUGAGCACCGAGACAGGCUUCGCCCUGCUGGGCGGCCACCCCUGCU
UCCUGACCACCCAGGACAUCCACCUGGGCGUGAACGAGAGCCUGACCGACACCGC
CCGCGUGCUGAGCAGCAUGGCCGACGCCGUGCUGGCCCGCGUGUACAAGCAGAGC
GACCUGGACACCCUGGCCAAGGAGGCCAGCAUCCCCAUCAUCAACGGCCUGAGCG
ACCUGUACCACCCCAUCCAGAUCCUGGCCGACUACCUGACCCUGCAGGAGCACUA
CAGCAGCCUGAAGGGCCUGACCCUGAGCUGGAUCGGCGACGGCAACAACAUCCUG
CACAGCAUCAUGAUGAGCGCCGCCAAGUUCGGCAUGCACCUGCAGGCCGCCACCC
CCAAGGGCUACGAGCCCGACGCCAGCGUGACCAAGCUGGCCGAGCAGUACGCCAA
GGAGAACGGCACCAAGCUGCUGCUGACCAACGACCCCCUGGAGGCCGCCCACGGC
GGCAACGUGCUGAUCACCGACACCUGGAUCAGCAUGGGCCAGGAGGAGGAGAAG
AAGAAGCGCCUGCAGGCCUUCCAGGGCUACCAGGUGACCAUGAAGACCGCCAAGG
UGGCCGCCAGCGACUGGACCUUCCUGCACUGCCUGCCCCGCAAGCCCGAGGAGGU
GGACGACGAGGUGUUCUACAGCCCCCGCAGCCUGGUGUUCCCCGAGGCCGAGAAC
CGCAAGUGGACCAUCAUGGCCGUGAUGGUGAGCCUGCUGACCGACUACAGCCCCC
AGCUGCAGAAGCCCAAGUUCUGA
Open Reading Frame of Construct 2260 (SEQ ID NO: 257)
AUGCUGGUAUUCAACCUGCGCAUCCUGCUGAACAACGCCGCCUUCCGCAACGGCC
ACAACUUCAUGGUGCGCAACUUCCGCUGCGGCCAGCCCCUGCAGAACCGGGUGCA
GCUGAAGGGCCGCGACCUGCUGACCCUGAAGAACUUCACCGGCGAGGAGAUCCGG
UACAUGCUGUGGCUGAGCGCCGACCUGAAGUUCCGCAUCAAGCAGAAGGGCGAG
UACCUGCCCCUGCUGCAGGGCAAGAGCCUGGGCAUGAUCUUCGAGAAGCGCAGCA
CCCGCACCCGCCUGAGCACCGAGACAGGCUUCGCCCUGCUGGGCGGCCACCCCUG
CUUCCUGACCACCCAGGACAUCCACCUGGGCGUGAACGAGAGCCUGACCGACACC
GCCCGCGUGCUGAGCAGCAUGGCCGACGCCGUGCUGGCCCGCGUGUACAAGCAGA
GCGACCUGGACACCCUGGCCAAGGAGGCCAGCAUCCCCAUCAUCAACGGCCUGAG
CGACCUGUACCACCCCAUCCAGAUCCUGGCCGACUACCUGACCCUGCAGGAGCAC
UACAGCAGCCUGAAGGGCCUGACCCUGAGCUGGAUCGGCGACGGCAACAACAUCC
UGCACAGCAUCAUGAUGAGCGCCGCCAAGUUCGGCAUGCACCUGCAGGCCGCCAC
CCCCAAGGGCUACGAGCCCGACGCCAGCGUGACCAAGCUGGCCGAGCAGUACGCC
AAGGAGAACGGCACCAAGCUGCUGCUGACCAACGACCCCCUGGAGGCCGCCCACG
GCGGCAACGUGCUGAUCACCGACACCUGGAUCAGCAUGGGCCAGGAGGAGGAGA
AGAAGAAGCGCCUGCAGGCCUUCCAGGGCUACCAGGUGACCAUGAAGACCGCCAA
GGUGGCCGCCAGCGACUGGACCUUCCUGCACUGCCUGCCCCGCAAGCCCGAGGAG
GUGGACGACGAGGUGUUCUACAGCCCCCGCAGCCUGGUGUUCCCCGAGGCCGAGA
ACCGCAAGUGGACCAUCAUGGCCGUGAUGGUGAGCCUGCUGACCGACUACAGCCC
CCAGCUGCAGAAGCCCAAGUUCUGA
Open Reading Frame of Construct 2262 (SEQ ID NO: 258)
AUGCUGGUAUUCAACCUGCGCAUCCUGCUGAACAACGCCGCCUUCCGCAACGGCC
ACAACUUCAUGGUGCGCAACUUCCGCUGCGGCCAGCCCCUGCAGAACCGGGUGCA
GCUGAAGGGCCGCGACCUGCUGACCCUGAAGAACUUCACCGGCGAGGAGAUCCGG
UACAUGCUGUGGCUGAGCGCCGACCUGAAGUUCCGCAUCAAGCAGAAGGGCGAG
UACCUGCCCCUGCUGCAGGGCAAGAGCCUGGGCAUGAUCUUCGAGAAGCGCAGCA
CCCGCACCCGCCUGAGCACCGAGACAGGCUUCGCCCUGCUGGGCGGCCACCCCUG
CUUCCUGACCACCCAGGACAUCCACCUGGGCGUGAACGAGAGCCUGACCGACACC
GCCCGCGUGCUGAGCAGCAUGGCCGACGCCGUGCUGGCCCGCGUGUACAAGCAGA
GCGACCUGGACACCCUGGCCAAGGAGGCCAGCAUCCCCAUCAUCAACGGCCUGAG
CGACCUGUACCACCCCAUCCAGAUCCUGGCCGACUACCUGACCCUGCAGGAGCAC
UACAGCAGCCUGAAGGGCCUGACCCUGAGCUGGAUCGGCGACGGCAACAACAUCC
UGCACAGCAUCAUGAUGAGCGCCGCCAAGUUCGGCAUGCACCUGCAGGCCGCCAC
CCCCAAGGGCUACGAGCCCGACGCCAGCGUGACCAAGCUGGCCGAGCAGUACGCC
AAGGAGAACGGCACCAAGCUGCUGCUGACCAACGACCCCCUGGAGGCCGCCCACG
GCGGCAACGUGCUGAUCACCGACACCUGGAUCAGCAUGGGCCAGGAGGAGGAGA
AGAAGAAGCGCCUGCAGGCCUUCCAGGGCUACCAGGUGACCAUGAAGACCGCCAA
GGUGGCCGCCAGCGACUGGACCUUCCUGCACUGCCUGCCCCGCAAGCCCGAGGAG
GUGGACGACGAGGUGUUCUACAGCCCCCGCAGCCUGGUGUUCCCCGAGGCCGAGA
ACCGCAAGUGGACCAUCAUGGCCGUGAUGGUGAGCCUGCUGACCGACUACAGCCC
CCAGCUGCAGAAGCCCAAGUUCUGA

1.-65. (canceled)

66. A method of expressing an ornithine transcarbamylase (OTC) enzyme in a mammal comprising administering a composition to the mammal, the composition comprising:
(a) an mRNA encoding the OTC enzyme; and
(b) a lipid formulation comprising an ionizable cationic lipid selected from

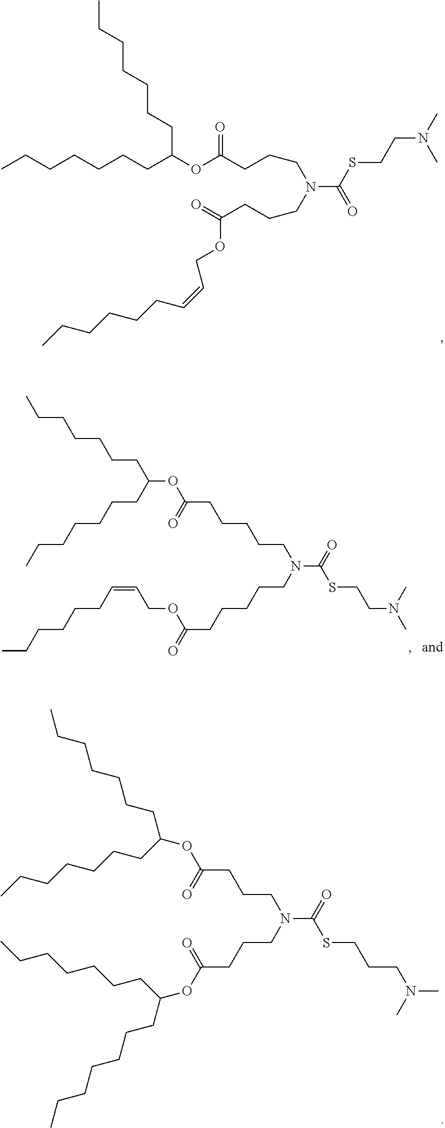

67.-71. (canceled)

72. The method of claim 66, wherein the OTC enzyme has at least 95% identity to the sequence of SEQ ID NO: 3 or SEQ ID NO:4.

73. The method of claim 66, wherein the mRNA comprises a coding region having a sequence selected from SEQ ID NOs: 254-258.

74. The method of claim 66, wherein the mRNA comprises:
(i) a 5′ untranslated region (5′ UTR) comprising the sequence of SEQ ID NO: 6; or
(ii) a 3′ untranslated region (3′ UTR) comprising a sequence selected from SEQ ID NOs: 16-22; or
(iii) a Kozak sequence having the sequence of SEQ ID NO: 23 or SEQ ID NO: 24; or
(iv) the 5′ UTR and the 3′ UTR; or
(v) the 5′ UTR and the Kozak sequence; or
(vi) the 3′ UTR and the Kozak sequence; or
(vii) the 5′ UTR, the 3′ UTR, and the Kozak sequence.

75. The method of claim 74, wherein the mRNA comprises a 3′ poly-adenosine (poly-A) tail comprising about 60 to about 125 consecutive adenine nucleotides.

76. The method of claim 66, wherein the mRNA comprises a 5′ cap.

77. The method of claim 76, wherein the 5′ cap is m⁷GpppAmpG having a structure of Formula Cap XI: 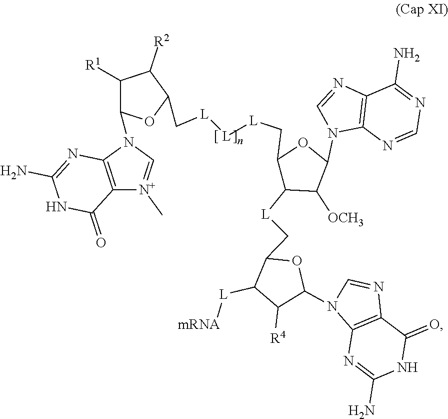
wherein R¹, R², and R⁴ are each OH; n is 1; each L is a phosphate linked by diester bonds;
and mRNA of Cap XI is the mRNA encoding the OTC enzyme linked at its 5′ end.

78. The method of claim 66, wherein the mRNA comprises a sequence selected from SEQ ID NOs: 1, 73, 119, and 251-253.

79. The method of claim 66, wherein about 1% to about 100% of uridine nucleotides of the mRNA are 5-methoxyuridine or N¹-methylpseudouridine.

80. The method of claim 79, wherein 100% of uridine nucleotides of the mRNA are 5-methoxyuridine or N¹-methylpseudouridine.

81. The method of claim 66, wherein the lipid formulation comprises lipid nanoparticles.

82. The method of claim 81, wherein:
(i) the lipid nanoparticles have an average particle size of less than about 100 nm; or
(ii) the lipid nanoparticles have an average particle size of about 55 nm to about 85 nm; or
(iii) the lipid nanoparticles encapsulate at least about 50% of the mRNA; or
(iv) the lipid nanoparticles encapsulate at least about 85% of the mRNA.

83. The method of claim 66, wherein the lipid formulation further comprises a helper lipid selected from dioleoylphosphatidyl ethanolamine (DOPE), dimyristoylphosphatidyl choline (DMPC), distearoylphosphatidylcholine (DSPC), dimyristoylphosphatidyl glycerol (DMPG), dipalmitoyl phosphatidylcholine (DPPC), and phosphatidylcholine (PC).

84. The method of claim 66, wherein the lipid formulation further comprises cholesterol.

85. The method of claim 66, wherein the lipid formulation further comprises a polyethylene glycol (PEG)-lipid conjugate.

86. The method of claim 66, wherein:
(i) the lipid formulation comprises about 48 mol % to about 66 mol % of the ionizable cationic lipid, about 2 mol % to about 12 mol % DSPC, about 25 mol % to about 42 mol % cholesterol, and about 0.5 mol % to about 3 mol % PEG2000-DMG; or
(ii) the lipid formulation comprises about 50 mol % to about 61 mol % of the ionizable cationic lipid, about 5 mol % to about 9 mol % DSPC, about 29 mol % to about 38 mol % cholesterol, and about 1 mol % to about 2 mol % PEG2000-DMG; or
(iii) the lipid formulation comprises about 56 mol % to about 58 mol % of the ionizable cationic lipid, about 6 mol % to about 8 mol % DSPC, about 31 mol % to about 34 mol % cholesterol, and about 1.25 mol % to about 1.75 mol % PEG2000-DMG.

87. The method of claim 66, wherein:
(i) the composition has a total lipid: mRNA weight ratio of about 50:1 to about 10:1; or
(ii) the composition has a total lipid: mRNA weight ratio of about 40:1 to about 20:1; or
(iii) the composition has a total lipid: mRNA weight ratio of about 35:1 to about 25:1; or
(iv) the composition has a total lipid: mRNA weight ratio of about 32:1 to about 28:1; or
(v) the composition has a total lipid: mRNA weight ratio of about 31:1 to about 29:1.

88. The method of claim 66, wherein the composition is characterized by one or more of the following:
(i) the composition comprises a HEPES buffer at a pH of about 7.4; or
(ii) the composition comprises a HEPES buffer at a pH of about 7.4 and at a concentration of about 7 mg/mL to about 15 mg/mL; or
(iii) the composition comprises about 2.0 mg/mL to about 4.0 mg/mL of NaCl; or
(iv) the composition comprises one or more cryoprotectants; or
(v) the composition comprises one or more cryoprotectants selected from sucrose, glycerol, or a combination of sucrose and glycerol; or
(vi) the composition comprises a combination of sucrose at a concentration of about 70 mg/mL to about 110 mg/mL and glycerol at a concentration of about 50 mg/mL to about 70 mg/mL.

89. The method of claim 66, wherein the mammal has ornithine transcarbamylase (OTC) deficiency prior to said administering.

90. The method of claim 66, wherein the mammal is a human.

91. The method of claim 66, wherein the OTC enzyme is expressed in a hepatocyte of the mammal after said administering.
